(19) United States
(12) Patent Application Publication
(10) Pub. No.: US 2021/0206818 A1
(43) Pub. Date: Jul. 8, 2021

(54) MESSENGER RIBONUCLEIC ACIDS FOR THE PRODUCTION OF INTRACELLULAR BINDING POLYPEPTIDES AND METHODS OF USE THEREOF

(71) Applicant: ModernaTX, Inc., Cambridge, MA (US)

(72) Inventors: Eric Yi-Chun HUANG, Boston, MA (US); Joshua P. FREDERICK, Charlestown, MA (US); Kristine MCKINNEY, Cambridge, MA (US); Christina HENDERSON, Stow, MA (US); Kahlin CHEUNG-ONG, Boston, MA (US); Joseph BOLEN, Cambridge, MA (US); Stephen Michael KELSEY, Brookline, MA (US); Michael MORIN, Salisbury, MA (US); Sushma GURUMURTHY, Sharon, MA (US); Kerry BENENATO, Sudbury, MA (US); Stephen HOGE, Brookline, MA (US); Iain MCFADYEN, Arlington, MA (US); Vladimir PRESNYAK, Manchester, NH (US)

(21) Appl. No.: 16/071,131

(22) PCT Filed: Jan. 20, 2017
(86) PCT No.: PCT/US2017/014441

§ 371 Date: Jul. 19, 2018

Related U.S. Application Data
(60) Provisional application No. 62/338,444, filed on May 18, 2016.
(60) Provisional application No. 62/286,295, filed on Jan. 22, 2016.
(60) Provisional application No. 62/286,299, filed on Jan. 22, 2016.
(60) Provisional application No. 62/286,308, filed on Jan. 22, 2016.

(51) Int. Cl. C07K 14/47; A61K 9/51; A61P 35/00

(52) U.S. Cl. CPC C07K 14/4705; A61K 9/5123; A61K 38/00; C07K 2319/70; A61P 35/00

(57) ABSTRACT

The invention features isolated mRNAs encoding at least one intracellular binding domain, including mRNAs comprising one or more modified nucleobase and preferably lacking an encoded scaffold polypeptide, and methods of using the same, for example, for inducing apoptosis and/or treating cancer (e.g., liver cancer or colorectal cancer).

### RELATED APPLICATIONS

This application claims the benefit of U.S. Provisional Patent Application Ser. No. 62/286,295 filed on Jan. 22, 2016; U.S. Provisional Patent Application Ser. No. 62/286,299 filed Jan. 22, 2016; U.S. Provisional Patent Application Ser. No. 62/286,308 filed Jan. 22, 2016; and U.S. Provisional Patent Application Ser. No. 62/338,444 filed on May 18, 2016. The entire contents of the above-referenced applications are incorporated herein by this reference.

### BACKGROUND OF THE INVENTION

A number of therapeutic tools exist for modulating the function of biological pathways and/or molecules that are involved in disease. These tools include, for example, small molecule inhibitors and therapeutic antibodies. However, many biological molecules (e.g., proteins) that are known to be involved in diseases, e.g., cancers, are not readily druggable using small molecule inhibitors or therapeutic antibodies. In particular, some intracellular targets, such as Bcl-2 family members (e.g., anti-apoptotic Bcl-2 family members), and members of the Hippo signaling pathway such as Yes-associated protein (YAP) and transcription co-activatory with PDZ-binding motif (TAZ), are difficult to target with small molecule inhibitors, and are also not accessible to therapeutic antibodies administered into the blood stream due to the permeability barrier of the cell's plasma membrane.

Clearly, therefore, there exists a need for new therapeutic compositions capable of modulating intracellular disease targets, including anti-apoptotic Bcl-2 family members, YAP and TAZ, and related methods for delivery these agents to cells to treat and prevent diseases such as cancers.

### SUMMARY OF THE INVENTION

The present disclosure provides compositions including isolated mRNAs encoding one or more intracellular binding polypeptides. In exemplary aspects, the mRNA constructs encoding the intracellular binding polypeptides do not encode a scaffold polypeptide for presenting the intracellular binding polypeptides, since it has been determined that such a scaffold polypeptide may not be necessary for some types of intracellular binding polypeptides, such as a BH3 domain, to function effectively, for example intracellularly, to modulate the activity of a target to which the BH3 domain(s) binds. In some aspects, the isolated mRNAs encode multiple BH3 domains, referred to herein as multimer constructs. In some aspects, the isolated mRNAs include one or more modified nucleobase and are referred to as modified mRNAs (mmRNAs). In some aspects, the isolated mRNAs are present in pharmaceutical compositions. In other aspects, the mRNAs are present in nanoparticles, e.g. lipid nanoparticles. The disclosure provides compositions including isolated mRNAs encoding at least one Bcl-2 homology 3 (BH3) domain, as well as methods of using such compositions, for example, for inducing apoptosis and/or treating cancer (e.g., liver cancer or colorectal cancer).

In a first aspect, the disclosure features a modified messenger RNA (mmRNA) encoding at least one Bcl-2 homology 3 (BH3) domain and lacking a scaffold polypeptide, wherein said mmRNA comprises one or more modified nucleobases. In one embodiment, the mmRNA encodes at least three BH3 domains. In one embodiment, the mmRNA encodes two to ten BH3 domains. In one embodiment, the mmRNA encodes three BH3 domains. In certain embodiments, the BH3 domains are selected from the group consisting of PUMA BH3, Bim BH3, Bad BH3, Noxa BH3, Beclin BH3 and a truncated BID protein containing a BH3 domain, and combinations thereof. In certain embodiments, the BH3 domains comprise the amino acid sequence of X₁X₂X₃X₄X₅X₆X₇X₈X₉DX₁₀X₁₁X₁₂, wherein X₁, X₅, X₈, and X₁₁ are any hydrophobic amino acid residue; X₂ and X₉ are Gly, Ala, or Ser; X₃, X₄, X₆, and X₇ are any amino acid residue; X₁₀ is Asp or Glu; and X₁₂ is Asn, His, Asp, or Tyr. In one embodiment, X₅ is leucine.

In certain aspects, the BH3 domain-encoding mRNAs of do not encode a scaffold polypeptide, other aspects provide mRNA which does comprise a scaffold polypeptide. Suitable scaffold polypeptides (e.g., mmRNA-encoded scaffolds) are described herein.

In certain embodiments, the mRNAs of the disclosure encode more than one BH3 domain, referred to herein as multimer BH3 domain constructs. In certain embodiments of the multimer BH3 domain constructs, the mRNA further encodes a linker located between each BH3 domain. The linker can be, for example, a cleavable linker or protease-sensitive linker. In certain embodiments, the linker is selected from the group consisting of F2A linker, P2A linker, T2A linker, E2A linker, and combinations thereof. In certain embodiments, the linker is an F2A linker. In certain embodiments, the linker is a GGGS linker. In certain embodiments, the multimer BH3 domain construct contains three BH3 domains with intervening linkers, having the structure: BH3 domain-linker-BH3 domain-linker-BH3 domain.

In certain embodiments, the mRNAs of the disclosure further comprise one or more microRNA (miR) binding sites. For example, in one embodiment, the mRNA comprises an miR122 binding site. In another embodiment, the mRNA comprises an miR142.3p binding site. In another embodiment, the mRNA comprises an miR122 binding site and an miR142.3p binding site.

In various embodiments, the mRNA constructs of the disclosure can further comprise at least one IRES sequence. In various embodiments, the mRNA constructs can further encode an epitope tag(s).

In another aspect, the disclosure provides an mRNA construct, such as a modified messenger RNA (mmRNA), encoding at least one truncated BID polypeptide that includes its Bcl-2 homology 3 (BH3) domain, wherein the mmRNA comprises one or more modified nucleobases. The truncated BID polypeptide containing the BH3 domain contains fewer amino acid residues than a full-length BID protein. For example, in one embodiment, the truncated BID (tBID) containing a BH3 domain consists of amino acids 61-195 of the BID protein. In another embodiment, the truncated BID (tBID) containing a BH3 domain consists of amino acids 77-195 of the BID protein. In certain embodiments, the truncated BID (tBID) polypeptide containing BH3 domain mRNA construct encodes multiple copies of the truncated BID polypeptide containing a BH3 domain, such as two to ten copies of the truncated BID polypeptide. In one embodiment, the mRNA construct encodes three copies of a truncated BID polypeptide. In embodiments in which the mRNA construct encodes multiple truncated BID polypeptides, each containing a BH3 domain, the construct can further encodes a linker located between each truncated BID polypeptide, such as the linkers described above.

In some embodiments, the BH3 domain-encoding mRNA encodes an amino acid sequence selected from the group consisting of SEQ ID NOs: 196, 197, 207, 225, 234, 236, 237, 291 and 294. In some embodiments, the BH3 domain-encoding mRNA comprises any one of SEQ ID NOs: 225, 244, 245, 246, 273, 281, 283, 284, 290 and 293.

In another aspect, the disclosure features a modified messenger RNA (mmRNA) encoding one or more intracellular binding peptides selected from the group consisting of: a TOPK inhibitory peptide, a SALL4 inhibitory peptide, a Ras inhibitory peptide, a p53 inhibitory peptide, a PP2a inhibitory peptide and a STAT3 inhibitory peptide, wherein the intracellular binding peptide lacks a scaffold polypeptide, and wherein said mmRNA comprises one or more modified nucleobases. In one embodiment, the mmRNA encodes at least three intracellular binding peptides (e.g., three TOPK inhibitory peptides, three a SALL4 inhibitory peptides, etc). In one embodiment, the mmRNA encodes two to ten intracellular binding peptides.

In certain embodiments of the multimer intracellular binding peptide constructs, the mRNA further encodes a linker located between each peptide (e.g., each TOPK-inhibitory peptide). The linker can be, for example, a cleavable linker or protease-sensitive linker. In certain embodiments, the linker is selected from the group consisting of F2A linker, P2A linker, T2A linker, E2A linker, and combinations thereof. The invention also relates to methods of using such compositions, for example, for treating cancer.

In another aspect, the disclosure provides compositions including isolated mRNAs encoding one or more YAP binding polypeptides, also referred to herein as YAP inhibitory peptides. In some aspects, an mRNA encodes a scaffold polypeptide for presenting the YAP binding polypeptide. In other aspects, an mRNA does not encode a scaffold polypeptide; rather, expression of the one or more YAP binding polypeptides intracellularly is sufficient for their function.

In another aspect, the disclosure features a modified messenger RNA (mmRNA) encoding at least one YAP inhibitory domain and lacking a scaffold polypeptide, wherein said mmRNA comprises one or more modified nucleobases. In one embodiment, the mmRNA encodes at least YAP inhibitory domains. In one embodiment, the mmRNA encodes two to ten YAP inhibitory domains. In one embodiment, the mmRNA encodes three YAP inhibitory domains. In certain embodiments, the YAP inhibitory domains are selected from the group set forth in SEQ ID NOs: 448-462, including combinations thereof.

In some embodiments, the mRNAs of the invention encode a scaffold polypeptide and one or more YAP inhibitory domains, wherein the mRNA is chemically modified to comprise one or more modified nucleobases. Suitable scaffold polypeptides (e.g., mmRNA-encoded scaffolds) are described herein.

In certain embodiments, the mRNAs of the disclosure encode more than one YAP inhibitory domain, referred to herein as multimer or tandem constructs. In certain embodiments of the multimer YAP inhibitory domain constructs, the mRNA further encodes a linker located between each YAP inhibitory domain. The linker can be, for example, a cleavable linker or protease-sensitive linker. In certain embodiments, the linker is selected from the group consisting of F2A linker, P2A linker, T2A linker, E2A linker, and combinations thereof. In certain embodiments, the linker is an F2A or a P2A linker. In certain embodiments, the linker is a GGGS linker. In certain embodiments, the multimer YAP inhibitory domain construct contains three YAP inhibitory domains with intervening linkers, having the structure: YAP inhibitory domain-linker-YAP inhibitory domain-linker-YAP inhibitory domain.

In some embodiments, the mRNA that selectively inhibits YAP encodes an amino acid sequence selected from the group consisting of: SEQ ID NOs: 481, 483, 488, 490 and 498. In some embodiments, the mRNA that selectively inhibits YAP comprises any one of SEQ ID NOs: 508, 510, 515, 517, 518 and 519.

In other aspects, the disclosure provides a lipid nanoparticle comprising an mRNA, such as a modified mRNA (mmRNA) of the invention. In certain embodiments, the lipid nanoparticle may include a cationic and/or ionizable lipid. In some embodiments, the cationic and/or ionizable lipid is DLin-KC2-DMA or DLin-MC3-DMA. In some embodiments, the lipid nanoparticle is a liposome. In certain embodiments, the lipid nanoparticle may further include a targeting moiety, such as a targeting moeity conjugated by a covalent linkage to the outer surface of the lipid nanoparticle.

The present disclosure provides a polynucleotide comprising an open reading frame (ORF) comprising mmRNA as described herein, (e.g., inhibitory YAP domain or BH3 polypeptide, e.g., a BH3 multimer, e.g., Puma BH3 multimer), wherein the uracil or thymine content of the ORF relative to the theoretical minimum uracil or thymine content of a nucleotide sequence encoding the at least one intracellular binding domain as described herein (% U_{TM} or % T_{TM}), is between about 100% and about 150%. In some embodiments, the % U_{TM} or % T_{TM} is between about 105% and about 145%, between about 105% and about 140%, between about 110% and about 140%, between about 110% and about 145%, between about 115% and about 135%, between about 105% and about 135%, between about 110% and about 135%, between about 115% and about 145%, or between about 115% and about 140%. In some embodiments, the % U_{TM} or % T_{TM} is between (i) 110%, 111%, 112%, 113%, 114%, 115%, 116%, 117%, or 118% and (ii) 132%, 133%, 134%, 135%, 136%, 137%, 138%, 139%, or 140%.

In some embodiments, the uracil or thymine content of the ORF relative to the uracil or thymine content of the corresponding wild-type ORF (% U_{WT} or % T_{WT}) is less than 100%. In some embodiments, the % U_{WT} or % T_{WT} is less than about 95%, less than about 90%, less than about 85%, less than 80%, less than 79%, less than 78%, less than 77%, less than 76%, less than 75%, less than 74%, or less than 73%. In some embodiments, the % U_{WT} or % T_{WT} is between 65% and 73%. In some embodiments, the uracil or thymine content in the ORF relative to the total nucleotide content in the ORF (% U_{TL} or % T_{TL}) is less than about 50%, less than about 40%, less than about 30%, less than about 20%, or less than about 14%. In some embodiments, the % U_{TL} or % T_{TL} in a Puma BH3 multimer is less than about 14%. In some embodiments, the % U_{TL} or % T_{TL} in a YAP inhibitory multimer is less than about 14%. In some embodiments, the % U_{TL} or % T_{TL} is between about 12% and about 13%. In some embodiments, the guanine content of the ORF with respect to the theoretical maximum guanine content of a nucleotide sequence encoding the at least one intracellular binding domain (% G_{TMX}) is at least 69%, at least 70%, at least 75%, at least about 80%, at least about 85%, at least about 90%, at least about 95%, or about 100%. In some embodiments, the % G_{TMX} is between about 70% and about 80%, between about 71% and about 79%, between about 71% and about 78%, or between about 71% and about 77%.

In some embodiments, the cytosine content of the ORF relative to the theoretical maximum cytosine content of a nucleotide sequence encoding the at least one intracellular binding domain (% C_{TMX}) is at least 65%, at least about 70%, at least about 75%, at least about 80%, at least about 85%, at least about 90%, at least about 95%, or about 100%. In some embodiments, the % C_{TMX} is between about 60% and about 80%, between about 62% and about 80%, between about 63% and about 79%, or between about 68% and about 76%. In some embodiments, the guanine and cytosine content (G/C) of the ORF relative to the theoretical maximum G/C content in a nucleotide sequence encoding the at least one intracellular binding domain (% G/C_{TMX}) is at least about 86%, at least about 90%, at least about 95%, or about 100%. In some embodiments, the % G/C_{TMX} is between about 80% and about 100%, between about 85% and about 99%, between about 90% and about 97%, or between about 91% and about 96%. In some embodiments, the G/C content in the ORF relative to the G/C content in the corresponding wild-type ORF (% G/C_{WT}) is at least 102%, at least 103%, at least 104%, at least 105%, at least 106%, at least 107%, at least 110%, at least 115%, or at least 120%. In some embodiments, the average G/C content in the 3rd codon position in the ORF is at least 20%, at least 21%, at least 22%, at least 23%, at least 24%, at least 25%, at least 26%, at least 27%, at least 28%, at least 29%, or at least 30% higher than the average G/C content in the 3rd codon position in the corresponding wild-type ORF.

In some embodiments, the ORF further comprises at least one low-frequency codon. In some embodiments of the polynucleotides disclosed herein the ORF is at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide described herein.

In some embodiments, the ORF has at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from the group consisting of SEQ ID NOs: 43 to 52, 74-105, 127-136, and 300-324.

In some embodiments, the polynucleotide is single stranded. In some embodiments, the polynucleotide is double stranded. In some embodiments, the polynucleotide is DNA. In some embodiments, the polynucleotide is RNA. In some embodiments, the polynucleotide is mRNA. In some embodiments, the polynucleotide comprises at least one chemically modified nucleobase, sugar, backbone, or any combination thereof. In some embodiments, the at least one chemically modified nucleobase is selected from the group consisting of pseudouracil (ψ), N1-methylpseudouracil (m1ψ), 2-thiouracil (s2U), 4′-thiouracil, 5-methylcytosine, 5-methyluracil, and any combination thereof. In some embodiments, the at least one chemically modified nucleobase is 5-methoxyuracil. In some embodiments, at least about 25%, at least about 30%, at least about 40%, at least about 50%, at least about 60%, at least about 70%, at least about 80%, at least about 90%, at least about 95%, at least about 99%, or 100% of the uracils are 5-methoxyuracils.

In some embodiments, the polynucleotide further comprises a miRNA binding site. In some embodiments, the miRNA binding site comprises one or more nucleotide sequences selected from SEQ ID NO: 298 and SEQ ID NO: 299. In some embodiments, the miRNA binding site binds to miR-142. In some embodiments, the miRNA binding site binds to miR-142-3p or miR-142-5p. In some embodiments, the miR142 comprises SEQ ID NO: 297.

In some embodiments, the polynucleotide further comprises a 5′ UTR. In some embodiments, the 5′ UTR comprises a nucleic acid sequence at least 90%, at least about 95%, at least about 96%, at least about 97%, at least about 98%, at least about 99%, or about 100% identical to a 5′UTR sequence selected from the group consisting of SEQ ID NO: 327-351, or any combination thereof. In some embodiments, the polynucleotide further comprises a 3′ UTR. In some embodiments, the 3′ UTR comprises a nucleic acid sequence at least about 90%, at least about 95%, at least about 96%, at least about 97%, at least about 98%, at least about 99%, or about 100% identical to a 3′UTR sequence selected from the group consisting of SEQ ID NO: 352-369, or any combination thereof. In some embodiments, the miRNA binding site is located within the 3′ UTR.

In some embodiments, the polynucleotide further comprises a 5′ terminal cap. In some embodiments, the 5′ terminal cap comprises a Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof. In some embodiments, the polynucleotide further comprises a poly-A region. In some embodiments, the poly-A region is at least about 10, at least about 20, at least about 30, at least about 40, at least about 50, at least about 60, at least about 70, at least about 80, or at least about 90 nucleotides in length. In some embodiments, the poly-A region has about 10 to about 200, about 20 to about 180, about 50 to about 160, about 70 to about 140, about 80 to about 120 nucleotides in length.

In some embodiments, the polynucleotide encodes an intracellular binding polypeptide that is fused to one or more heterologous polypeptides. In some embodiments, the one or more heterologous polypeptides increase a pharmacokinetic property of the intracellular binding polypeptide. In some embodiments, upon administration to a subject, the polynucleotide has (i) a longer plasma half-life; (ii) increased expression of the polypeptide encoded by the ORF; (iii) a lower frequency of arrested translation resulting in an expression fragment; (iv) greater structural stability; or (v) any combination thereof, relative to a corresponding polynucleotide encoding the at least one intracellular binding domain.

In some embodiments, the polynucleotide comprises (i) a 5′-terminal cap; (ii) a 5′-UTR; (iii) an ORF encoding at least one intracellular binding domain; (iv) a 3′-UTR; and (v) a poly-A region. In some embodiments, the 3′-UTR comprises a miRNA binding site.

The present disclosure also provides a method of producing a polynucleotide of the present invention, the method comprising modifying an ORF encoding at least one intracellular binding polypeptide by substituting at least one uracil nucleobase with an adenine, guanine, or cytosine nucleobase, or by substituting at least one adenine, guanine, or cytosine nucleobase with a uracil nucleobase, wherein all the substitutions are synonymous substitutions. In some embodiments, the method further comprises replacing at least about 90%, at least about 95%, at least about 99%, or about 100% of uracils with 5-methoxyuracils.

The present disclosure also provides a composition comprising (a) a polynucleotide of the invention; and (b) a delivery agent. In some embodiments, the delivery agent comprises a lipidoid, a liposome, a lipoplex, a lipid nanoparticle, a polymeric compound, a peptide, a protein, a cell, a nanoparticle mimic, a nanotube, or a conjugate. In some embodiments, the delivery agent comprises a lipid nanoparticle. In some embodiments, the lipid nanoparticle comprises a lipid selected from the group consisting of
3-(didodecylamino)-N1,N1,4-tridodecyl-1-piperazineethanamine (KL10),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA),
heptatriaconta-6,9,28,31-tetraen-19-yl 4-(dimethylamino)butanoate (DLin-MC3-DMA),
2,2-dilinoleyl-4-(2-dimethylaminoethyl)-[1,3]-dioxolane (DLin-KC2-DMA),
1,2-dioleyloxy-N,N-dimethylaminopropane (DODMA), (13Z,165Z)—N,N-dimethyl-3-nonydocosa-13-16-dien-1-amine (L608),
2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA),
(2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)),
(2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), and any combinations thereof.

In some embodiments, the delivery agent comprises a compound having the formula (I)

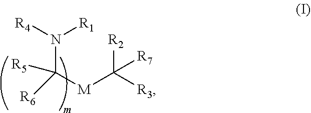

or a salt or stereoisomer thereof, wherein
   R₁ is selected from the group consisting of C₅₋₂₀ alkyl, C₅₋₂₀ alkenyl, —R*YR″, —YR″, and —R″M′R′;
   R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
   R₄ is selected from the group consisting of a C₃₋₆ carbocycle, —(CH₂)ₙQ, —(CH₂)ₙCHQR,
—CHQR, —CQ(R)₂, and unsubstituted C₁₋₆ alkyl, where Q is selected from a carbocycle, heterocycle,
—OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5;
   each R₅ is independently selected from the group consisting of C₁₋₃ alkyl, C₂₋₃ alkenyl, and H;
   each R₆ is independently selected from the group consisting of C₁₋₃ alkyl, C₂₋₃ alkenyl, and H;
   M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
      R₇ is selected from the group consisting of C₁₋₃ alkyl, C₂₋₃ alkenyl, and H;
   each R is independently selected from the group consisting of C₁₋₃ alkyl, C₂₋₃ alkenyl, and H;
   each R′ is independently selected from the group consisting of C₁₋₁₈ alkyl, C₂₋₁₈ alkenyl, —R*YR″, —YR″, and H;
   each R″ is independently selected from the group consisting of C₃₋₁₄ alkyl and C₃₋₁₄ alkenyl;
   each R* is independently selected from the group consisting of C₁₋₁₂ alkyl and C₂₋₁₂ alkenyl;
   each Y is independently a C₃₋₆ carbocycle;
   each X is independently selected from the group consisting of F, Cl, Br, and I; and
   m is selected from 5, 6, 7, 8, 9, 10, 11, 12, and 13; and provided when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

The present disclosure also provides a composition comprising a nucleotide sequence encoding at least one intracellular binding domain and a delivery agent, wherein the delivery agent comprises a compound having the formula (I)

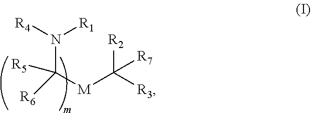

or a salt or stereoisomer thereof, wherein
   R₁ is selected from the group consisting of C₅₋₂₀ alkyl, C₅₋₂₀ alkenyl, —R*YR″, —YR″, and —R″M′R′;
   R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
   R₄ is selected from the group consisting of a C₃₋₆ carbocycle, —(CH₂)ₙQ, —(CH₂)ₙCHQR,
—CHQR, —CQ(R)₂, and unsubstituted C₁₋₆ alkyl, where Q is selected from a carbocycle, heterocycle,
—OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5;
   each R₅ is independently selected from the group consisting of C₁₋₃ alkyl, C₂₋₃ alkenyl, and H;
   each R₆ is independently selected from the group consisting of C₁₋₃ alkyl, C₂₋₃ alkenyl, and H;
   M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
      R₇ is selected from the group consisting of C₁₋₃ alkyl, C₂₋₃ alkenyl, and H;
   each R is independently selected from the group consisting of C₁₋₃ alkyl, C₂₋₃ alkenyl, and H;
   each R′ is independently selected from the group consisting of C₁₋₁₈ alkyl, C₂₋₁₈ alkenyl, —R*YR″, —YR″, and H;
   each R″ is independently selected from the group consisting of C₃₋₁₄ alkyl and C₃₋₁₄ alkenyl;
   each R* is independently selected from the group consisting of C₁₋₁₂ alkyl and C₂₋₁₂ alkenyl;
   each Y is independently a C₃₋₆ carbocycle;
   each X is independently selected from the group consisting of F, Cl, Br, and I; and
   m is selected from 5, 6, 7, 8, 9, 10, 11, 12, and 13; and provided when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, the compound is of Formula (IA):

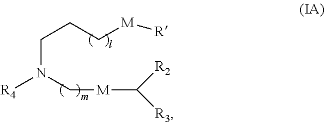

or a salt or stereoisomer thereof, wherein
   l is selected from 1, 2, 3, 4, and 5;
   m is selected from 5, 6, 7, 8, and 9;
   M₁ is a bond or M′;
   R₄ is unsubstituted C₁₋₃ alkyl, or —(CH₂)ₙQ, in which n is 1, 2, 3, 4, or 5 and Q is OH, —NHC(S)N(R)₂, or —NHC(O)N(R)₂;
   M and M′ are independently selected
   from —C(O)O—, —OC(O)—, —C(O)N(R′)—, —P(O)(OR′)O—,
   an aryl group, and a heteroaryl group; and
      R₂ and R₃ are independently selected from the group consisting of H, C₁₋₁₄ alkyl, and C₂₋₁₄ alkenyl.

In some embodiments, m is 5, 7, or 9.

In some embodiments, the compound is of Formula (II):

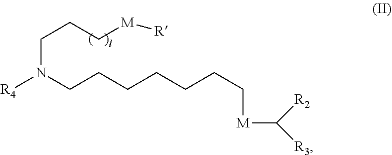

or a salt or stereoisomer thereof, wherein
   l is selected from 1, 2, 3, 4, and 5;
   M₁ is a bond or M′;
   R₄ is unsubstituted C₁₋₃ alkyl, or —(CH₂)ₙQ, in which n is 2, 3, or 4 and Q is OH, —NHC(S)N(R)₂, or —NHC(O)N(R)₂;
   M and M′ are independently selected
   from —C(O)O—, —OC(O)—, —C(O)N(R′)—, —P(O)(OR′)O—,
   an aryl group, and a heteroaryl group; and
      R₂ and R₃ are independently selected from the group consisting of H, C₁₋₁₄ alkyl, and C₂₋₁₄ alkenyl.

In some embodiments, M1 is M′. In some embodiments, M and M′ are independently —C(O)O— or —OC(O)—. In some embodiments, 1 is 1, 3, or 5. In some embodiments, the compound is selected from the group consisting of Compound 1 to Compound 147, salts and stereoisomers thereof, and any combination thereof.

In some embodiments, the compound is of the Formula (IIa),

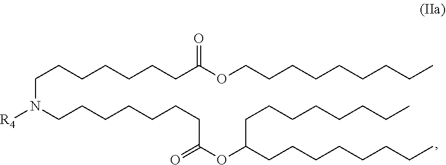

or a salt or stereoisomer thereof.

In some embodiments, the compound is of the Formula (IIb),

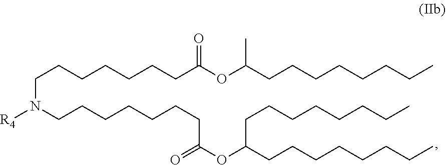

or a salt or stereoisomer thereof.

In some embodiments, the compound is of the Formula (IIc) or (IIe),

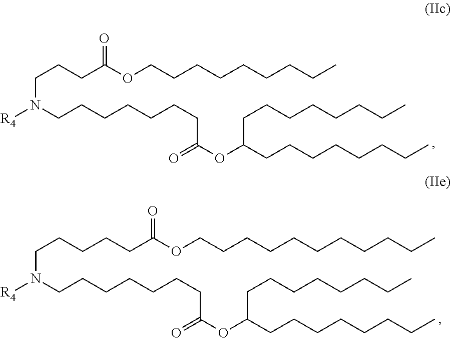

or a salt or stereoisomer thereof.

In some embodiments, R₄ is selected from —(CH₂)ₙQ and —(CH₂)ₙCHQR. In some embodiments, the compound is of the Formula (IId),

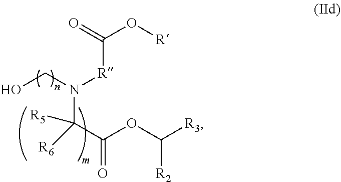

or a salt or stereoisomer thereof,
wherein R₂ and R₃ are independently selected from the group consisting of C₅₋₁₄ alkyl and C₅₋₁₄ alkenyl, n is selected from 2, 3, and 4, and R′, R″, R₅, R₆ and m are as defined in claim 60 or 61.

In some embodiments, wherein R₂ is C₈ alkyl. In some embodiments, R₃ is C₅ alkyl, C₆ alkyl, C₇ alkyl, C₈ alkyl, or C₉ alkyl. In some embodiments, m is 5, 7, or 9. In some embodiments, each R₅ is H. In some embodiments, each R₆ is H.

In some embodiments, the composition disclosed herein is a nanoparticle composition. In some embodiments, the delivery agent further comprises a phospholipid. In some embodiments, the phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanolamine (DOPE),
1,2-diphytanoyl-sn-glycero-3-phosphoethanolamine (ME 16:0 PE),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG), sphingomyelin, and any mixtures thereof.

In some embodiments, the delivery agent further comprises a structural lipid. In some embodiments, the structural lipid is selected from the group consisting of cholesterol, fecosterol, sitosterol, ergosterol, campesterol, stigmasterol, brassicasterol, tomatidine, ursolic acid, alpha-tocopherol, and any mixtures thereof.

In some embodiments, the delivery agent further comprises a PEG lipid. In some embodiments, the PEG lipid is selected from the group consisting of a PEG-modified phosphatidylethanolanine, a PEG-modified phosphatidic acid, a PEG-modified ceramide, a PEG-modified dialkylamine, a PEG-modified diacylglycerol, a PEG-modified dialkylglycerol, and any mixtures thereof.

In some embodiments, the delivery agent further comprises an ionizable lipid selected from the group consisting of
3-(didodecylamino)-N1,N1,4-tridodecyl-1-piperazineethanamine (KL10),
N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA),
heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA),
1,2-dioleyloxy-N,N-dimethylaminopropane (DODMA),
2-({8-[(3β)-cholest-5-en-3-yloxy]octyl}oxy)-N,N-dimethyl-3-[(9Z,12Z)-octadeca-9,1 2-dien-1-yloxy]propan-1-amine (Octyl-CLinDMA),
(2R)-2-({8-[(3β)-cholest-5-en-3-yloxy]octyl}oxy)-N,N-dimethyl-3-[(9Z,12Z)-octadec
a-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadec a-9,12-dien-1-yloxy]propan-1-amine (Octyl-CLinDMA (2S)).

In some embodiments, the delivery agent further comprises a phospholipid, a structural lipid, a PEG lipid, or any combination thereof.

In some embodiments, the composition is formulated for in vivo delivery. In some embodiments, the composition is formulated for intramuscular, subcutaneous, or intradermal delivery.

The present disclosure also provides a host cell comprising a polynucleotide of the invention. In some embodiments, the host cell is a eukaryotic cell. The present disclosure also provides a vector comprising a polynucleotide of the invention. Also provided is a method of making a polynucleotide of the invention comprising synthesizing the polynucleotide enzymatically or chemically. The present disclosure also provides a polypeptide encoded by a polynucleotide of the invention, a composition comprising a polynucleotide of the invention, a host cell comprising a polynucleotide of the invention, a vector comprising a polynucleotide of the invention, or produced by the method of making disclosed herein.

In another aspect, the disclosure provides a pharmaceutical composition comprising any one of the preceding mRNAs or nanoparticles, e.g., lipid nanoparticles, and a pharmaceutically acceptable diluent, carrier or excipient.

In another aspect, the disclosure provides a method for inducing apoptosis in a cell, the method including contacting the cell with any one of the preceding mRNA constructs (e.g., modified mRNA constructs), or preceding nanoparticles (e.g., a lipid nanoparticle) or preceding pharmaceutical compositions, thereby inducing apoptosis. The contacting can occur in vitro or in vivo. In some embodiments, the cell is a cancer cell. In some embodiments, the cancer cell is a liver cancer cell. In some embodiments, the liver cancer cell is a hepatocellular carcinoma cell. In some embodiments, the cancer cell is a colorectal cancer cell. In some embodiments, the colorectal cancer cell is in a primary tumor or a metastasis. In some embodiments, the cancer cell is a hematopoietic cell. In some embodiments, the cancer cell is a myeloid cell. In some embodiments, the cancer cell is a hematopoietic stem cell (e.g., a hematopoetic stem cell from bone marrow, an erythroid stem cell, a myeloid stem cell, a thrombocytic stem cell). In any of the preceding embodiments, the cell may be a human cell.

In another aspect, the disclosure provides a method for treating a subject having cancer, the method including providing or administering an effective amount of any one of the preceding mRNA constructs (e.g., modified mRNA constructs), or preceding nanoparticles (e.g., a lipid nanoparticle) or preceding pharmaceutical compositions to the subject. In some embodiments, the cancer is liver cancer or colorectal cancer. In some embodiments, the liver cancer is hepatocellular carcinoma. In some embodiments, the colorectal cancer is a primary tumor or a metastasis. In some embodiments, the cancer is a hematopoietic cancer. In some embodiments, the cancer is an acute myeloid leukemia, a chronic myeloid leukemia, a chronic myelomonocytic leukemia, a myelodystrophic syndrome (including refractory anemias and refractory cytopenias) or a myeloproliferative neoplasm or disease (including polycythemia vera, essential thrombocytosis and primary myelofibrosis). In some embodiments, the lipid nanoparticle or isolated mRNA (or pharmaceutical composition) is administered to the patient parenterally.

In certain embodiments, the cell is also contacted with, or the subject is also provided with an mRNA that selectively inhibits MCL1, or a pharmaceutical composition comprising the mmRNA that selectively inhibits MCL1, wherein the mRNA in the pharmaceutical composition is optionally in a lipid nanoparticle. In certain embodiments, the mRNA that selectively inhibits MCL1 encodes an amino acid sequence selected from the group consisting of SEQ ID NOs: 117-126.

In another aspect, the disclosure provides a lipid nanoparticle encapsulating an modified mRNA of the invention, wherein the lipid nanoparticle comprises a cationic lipid, a PEG-modified lipid, a sterol and a non-cationic lipid. In one embodiment, the cationic lipid is selected from the group consisting of 2,2-dilinoleyl-4-methylaminoethyl-[1,3]-dioxolane (DLin-KC2-DMA), dilinoleyl-methyl-4-dimethylaminobutyrate (DLin-MC3-DMA) and di((Z)-non-2-en-1-yl) 9-((4-(dimethylamino)butanoyl)oxy)heptadecanedioate (L319). In one embodiment, the cationic lipid nanoparticle has a molar ratio of about 20-60% cationic lipid, about 5-25% non-cationic lipid, about 25-55% sterol and about 0.5-15% PEG-modified lipid. In one embodiment, the cationic lipid is an ionizable cationic lipid and the non-cationic lipid is a neutral lipi, and the sterol is cholesterol. In one embodiment, the the open reading frame of the encapsulated mmRNA is codon-optimized. In one embodiment, the nanoparticle has a polydiversity value of less than 0.4. In one embodiment, the nanoparticle has a net neutral charge at a neutral pH. In one embodiment, the nanoparticle has a mean diameter of 50-200 nm. In one embodiment, at least 80% of the uracils in the open reading frame of the encapsulated mmRNA have a chemical modification. In one embodiment, 100% of the uracils in the open reading frame of the encapsulated mmRNA have a chemical modification. In one embodiment, the chemical modification is in the 5-position of the uracils.

### BRIEF DESCRIPTION OF THE DRAWINGS

FIG. 1 is a photograph of a Western blot showing the results of Wheat Germ Lysate (WGL) cell free translation of mmRNA constructs encoding one BH3 domain (constructs 183532 and 183533) or three BH3 domains with intervening F2A linkers (construct 183534), demonstrating detection of mono-, di- and trimeric BH3 domain species by translation of the multimeric BH3 construct.

FIG. 2 is a graph showing that Hep3B human hepatocellular carcinoma cells transfected with mmRNA constructs encoding multimeric BH3 domains undergo apoptosis.

FIGS. 3A-3C are graphs showing that Hep3B human hepatocellular carcinoma cells transfected with lipid nanoparticles (LNPs) containing mmRNA constructs encoding multimeric BH3 domains undergo apoptosis. FIG. 3A shows results at 12 hours post-transfection using Caspase 3/7 reagent staining; FIG. 3B shows results at 24 hours post-transfection using Caspase 3/7 reagent staining; FIG. 3C shows results at 24 hours post transfection using Cell Titer Glo (CTG) assay.

FIG. 4 is a graph showing that Hep3B human hepatocellular carcinoma cells transfected with mmRNA constructs encoding a truncated BID protein (amino acids 61-195, including its BH3 domain) undergo apoptosis.

FIG. 5 is a graph showing that Hep3B human hepatocellular carcinoma cells transfected with mmRNA constructs encoding a truncated BID protein (amino acids 77-195, including its BH3 domain) undergo apoptosis.

FIGS. 6A-D are graphs showing the synergistic pro-apoptotic effect of targeting MCL1 in combination with SQT-PUMA-BH3 or SQT-Bad-BH3 in hepatocellular carcinoma cells (HCC) but not in primary hepatocytes. FIGS. 6A and 6C show Hep3B HCC cells. FIGS. 6B and 6D show primary hepatocytes. FIGS. 6A and 6B show SQT-PUMA-BH3. FIGS. 6C and 6D show SQT-Bad-BH3.

FIGS. 7A-B are bar graphs showing the synergistic pro-apoptotic effects of anti-MCL1 mRNA constructs in combination with SQT-PUMA-BH3. FIG. 7A shows anti-MCL1 mRNA at 50 ng. FIG. 7B shows anti-MCL mRNA at 12.5 ng.

FIG. 8 is a schematic diagram of the design of the indicated BH3 multimer constructs, as well as the SQT-PUMA-BH3 scaffolded construct and PUMA-BH3 monomer construct used as controls (top row). The middle row illustrates the final peptide expression products (shown after arrow) of the self-cleaving multimer constructs. The bottom row illustrates the multimer constructs containing GGGS linkers, which are not cleavable so these constructs remain as trimers or dimers following expression (shown after arrow).

FIG. 9 is a graph comparing the apoptosis of Hep3B human hepatocellular carcinoma cells transfected with mmRNA constructs encoding self-cleaving multimeric BH3 domain constructs (containing F2A or P2A cleavable linkers) or encoding uncleavable multimeric BH3 domain constructs (containing uncleavable GGGS linkers). Scaffolded SQT constructs were used as the positive (SQT-PUMA-BH3) and negative (SQT-dummy) controls.

FIG. 10 is a graph showing the apoptosis of Hep3B human hepatocellular carcinoma cells transfected with lipid nanoparticles (LNPs) containing an mmRNA construct encoding an uncleavable multimeric BH3 domain construct (containing uncleavable GGGS linkers), as compared to cells treated with the scaffolded SQT construct positive control (SQT-PUMA-BH3).

FIGS. 11A and 11B are bar graphs showing apoptosis of metastatic lymphoma HGC27 cells (FIG. 11A) and lung carcinoma A549 cells (FIG. 11B) following transfection of YAP inhibitory mRNA constructs into the cells.

FIG. 12 is a photograph of a Western blot analysis of immunoprecipitated cell lysates from metastatic lymphoma HGC27 cells transfected with YAP inhibitory mRNA constructs, demonstrating binding of YAP inhibitory constructs to TEAD4 transcription factor in the cells.

FIG. 13 is a bar graph showing relative mRNA levels of CTGF and CYR61 following transfection of YAP inhibitory mRNA constructs in HGC27 cells, as compared to cells treated with YAP-CMV plasmid or eGFP.

FIG. 14 provides images showing apoptosis of NCI-N87 cells via YOYO-3 staining, 72 hours after transfection with YAP inhibitory mRNA constructs, as compared to cells untreated or treated with scaffolded SQT construct positive control.

### DETAILED DESCRIPTION

Intracellular delivery of relatively small therapeutic polypeptides that can specifically bind to an intracellular target is one approach to modulate such intracellular targets. For example, inhibition of anti-apoptotic Bcl-2 family proteins in cancer cells may induce apoptosis in cancer cells, including cancer cells that are resistant to conventional chemotherapies. In principle, introduction of an mRNA encoding such a therapeutic polypeptide into the cell may lead to translation of the therapeutic polypeptide within the cell, allowing it to modulate its intracellular target(s). Delivery of mRNA encoding such a therapeutic polypeptide has advantages over other nucleic acid delivery approaches known in the art, such as viruses (e.g., retroviruses), because delivery of mRNA typically does not lead to integration of the nucleic acid into the host cell's genome, allowing transient expression of the nucleic acid. However, the delivery of therapeutic RNAs to cells is generally considered difficult, for example, due to the relative instability and low cell permeability of RNAs.

In some aspects, the disclosure provides compositions, such as isolated mRNAs encoding one or more intracellular binding peptides, such as BH3 domains. In some aspects, the mRNA construct encoding the intracellular binding peptide does not encode a scaffold polypeptide for presenting the peptide, since it has been determined for some intracellular binding peptides, such as BH3 domains, that such a scaffold polypeptide may not be necessary for the domain to function effectively, for example intracellularly, to modulate the activity of a target to which the domain(s) binds. In some embodiments, the isolated mRNAs encode multiple intracellular binding peptides, such as BH3 domains, referred to herein as multimer constructs. In some embodiments, the isolated mRNAs include one or more modified nucleobase and are referred to as modified mRNAs (mmRNAs).

In other aspects, the mmRNA encodes one or more intracellular binding peptides selected from the group consisting of: a TOPK inhibitory peptide, a SALL4 inhibitory peptide, a Ras inhibitory peptide, a p53 inhibitory peptide, a PP2a inhibitory peptide, a STAT3 inhibitory peptide and a YAP inhibitory peptide, wherein the intracellular binding peptide lacks a scaffold polypeptide, and wherein said mmRNA comprises one or more modified nucleobases. In other aspects, the mmRNA encodes one or more intracellular binding peptides selected from the group consisting of: a TOPK inhibitory peptide, a SALL4 inhibitory peptide, a Ras inhibitory peptide, a p53 inhibitory peptide, a PP2a inhibitory peptide, a STAT3 inhibitory peptide and a YAP inhibitory peptide, wherein the intracellular binding peptide is linked to a scaffold polypeptide, and wherein said mmRNA comprises one or more modified nucleobases. In some aspects, the mmRNA encodes at least three intracellular binding peptides (e.g., three YAP inhibitory peptides, three TOPK inhibitory peptides, three SALL4 inhibitory peptides, etc). In some aspects, the mmRNA encodes two to ten intracellular binding peptides. In some aspects, the mmRNA encodes at least one YAP inhibitory peptide, optionally two or three YAP binding peptides, operably linked via a peptide linker, optionally with a scaffold polypeptide.

In addition, the present disclosure provides nanoparticles, e.g., lipid nanoparticles, that contain mRNAs encoding one or more intracellular binding peptides, for example, BH3 domains, as well as pharmaceutical composition comprising any of these mRNAs or nanoparticles, e.g., lipid nanoparticles. The disclosure further provides methods of inducing apoptosis in a cell by contacting the cell with a composition of the disclosure (e.g., an isolated mRNA or a lipid nanoparticle). The disclosure also provides methods of treating a patient suffering from cancer that involve administration of a composition of the invention, e.g., in a pharmaceutical composition further comprising one or more pharmaceutically acceptable carriers, diluents or excipients.

### BH3 Domains

In various embodiments, an mRNA of the disclosure encodes one or more BH3 domains. In particular embodiments, an mRNA of the disclosure does not encode a scaffold polypeptide for presenting the BH3 domain(s); rather, expression of the one or more BH3 domains intracellularly is sufficient for their function. In particular embodiments, the isolated mRNAs encode multiple BH3 domains, referred to herein as multimer constructs. In one embodiment, the mRNA encodes at least three BH3 domains. In one embodiment, the mRNA encodes two to ten BH3 domains. In one embodiment, the mRNA encodes three BH3 domains. In other embodiments, the mRNA encodes 2, 4, 5, 6, 7, 8, 9 or 10 BH3 domains.

In some embodiments, the BH3 domain-encoding mRNA encodes an amino acid sequence of any one of SEQ ID NOs: 148, 149, 150, 159, 177, 185, 187, 188, 289 and 292. In some embodiments, the BH3 domain-encoding mRNA encodes an amino acid sequence of any one of SEQ ID NOs: 196, 197, 207, 225, 234, 236, 237, 291 and 294. In some embodiments, the BH3 domain-encoding mRNA comprises any one of SEQ ID NOs: 225, 244, 245, 246, 273, 281, 283, 284, 290 and 293.

When a construct contains multiple BH3 domains, the construct can contain multiple copies of the same BH3 domain or, alternatively, can contain a combination of two or more different BH3 domains. In certain embodiments, the BH3 domains are selected from the group consisting of PUMA BH3, Bim BH3, Bad BH3, Noxa BH3, truncated BID polypeptide containing a BH3 domain, and combinations thereof.

In some embodiments, the BH3 domain is a human BH3 domain. In other embodiments, the BH3 domain may be from a non-human species, e.g., Caenorhabditis elegans, rodents (e.g., mice and rats), or non-human primates. Typically, a BH3 domain is derived from a pro-apoptotic Bcl-2 family member, including from an effector pro-apoptotic Bcl-2 family member (e.g., BAK or BAX) or from a BH3-only family member (e.g., BID, BIM, BAD, BIK, BMF, bNIP3, HRK, Noxa, and PUMA). In particular embodiments, the BH3 domain is a BH3 domain derived from a BH3-only family member. Without wishing to be bound by theory, it is known in the art that the balance of pro-apoptotic Bcl-2 family proteins and anti-apoptotic Bcl-2 family proteins in a cell is important for regulation of apoptosis. Structural studies have shown that the BH3 domain of BH3-only proteins can bind as an amphipathic helix in a surface-exposed hydrophobic groove of an anti-apoptotic Bcl-2 family member (see, for example, Day et al., J. Mol. Biol. 380:958-971, 2008). The invention features methods of inducing apoptosis that involve introducing an mRNA encoding one or more BH3 domains into a cell under conditions permissive for expression of the one or more BH3 domains.

In some embodiments, a BH3 domain may directly bind to a Bcl-2 family protein. For example, in some embodiments, a BH3 domain may directly bind to a pro-apoptotic Bcl-2 family protein. In some embodiments, the pro-apoptotic Bcl-2 family protein is Bax and/or Bak. In some embodiments, a BH3 domain may directly interact with an anti-apoptotic Bcl-2 family protein. In some embodiments, the anti-apoptotic Bcl-2 family protein may BCL-2, BCL-XL, BCL-w, MCL-1 or BCL2-related protein A1 (BCL2A1).

In some embodiments, a BH3 domain is derived from a BH3-only family member. In some embodiments, a BH3 domain as used herein may include an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to the amino acid sequence of X₁X₂X₃X₄X₅X₆X₇X₈X₉DX₁₀X₁₁X₁₂, wherein X₁, X₅, X₈, and X₁₁ are, independently, any hydrophobic residue, X₂ and X₉ are, independently, Gly, Ala, or Ser, X₃, X₄, X₆, and X₇ are, independently, any amino acid residue, X₁₀ is Asp or Glu, and X₁₂ is Asn, His, Asp, or Tyr. In some embodiments, a hydrophobic residue is Leu, Ala, Val, Ile, Pro, Phe, Met or Trp. In some embodiments, X₅ is Leu.

In some embodiments, a BH3 domain as used herein may include an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to any one of SEQ ID NOs: 1-26. In some embodiments, the BH3 domain includes an amino acid sequence selected from the group consisting of SEQ ID NOs: 1-26, as shown in Table 1, which also indicates the name, UniProt sequence identifier, and amino acid residues. Illustrative BH3 domains that may be used according to the present invention are also described in Lomonosova and Chinnadurai, Oncogene (2009) 27, S2-S19, which is hereby incorporated by reference in its entirety.

TABLE 1
Illustrative BH3 Domains

| SEQ | | |
|---|---|---|
| ID | BH3 Domain | |
| NO | Sequence | Source Protein (UniProt) |
| 1 | LALRLACIGDEMDVS | BIK>Q13323\|57-71 |
| 2 | EKAELLQGGDLLRQR | BNIP1>Q12981\|110-124 |
| 3 | VESILKKNSDWIWDW | BNIP3>Q12983\|106-120 |
| 4 | EVEALKKSADWVSDW | BNIP3L>O60238\|120-134 |
| 5 | TAARLKALGDELHQR | HRK>O00198\|33-47 |
| 6 | IAQELRRIGDEFNAY | BIMe1>O43521\|148-162 |
| 7 | YGRELRRMSDEFVDS | BAD>Q92934\|110-124 |
| 8 | IARHLAQVGDSMDRS | BID>P55957\|86-100 |
| 9 | IARKLQCIADQFHRL | BMF>Q96LC9\|133-147 |
| 10 | CATQLRRFGDKLNFR | NOXA>Q13794\|25-39 |
| 11 | IGAQLRRMADDLNAQ | PUMA>Q9BXH1\|137-151 |
| 12 | LSRRLKVTGDLFDIM | Beclin1>Q11457\|112-126 |
| 13 | IVELLKYSGDQLERK | BCL-Gs>Q9BZR8-2\|212-226 |
| 14 | ALETLRRVGDGVQRN | MCL-1S>Q07820-2\|209-223 |
| 15 | IGSKLAAMCDDFDAQ | EGL-1>O61667\|69-83 |
| 16 | IGRKLTVMCDEFDSE | CED-13>Q9TYO6\|55-69 |
| 17 | NIRRLRALADGVQKV | ApoLl>O14791\|154-168 |
| 18 | NIDKLRALADDIDKT | ApoL6>Q9BWW8\|56-70 |
| 19 | MVTLLPIEGQEIHFF | BRCC2>Q8IZY5\|1-115 |
| 20 | PTVPLPSETDGYVAP | HER2>P04626\|1116-1130 |
| 21 | PQRYLVIQGDDRMKL | HER4>Q1503\|981-995 |
| 22 | TVGELSRALGHENGS | MAP1>Q96BY2\|116-130 |
| 23 | VGQLLQDMGDDVYQQ | MULE>Q7Z6X7\|1976-1990 |
| 24 | LHEVLNGLLDRPDWE | SPHK2>Q9NRA0\|250-264 |
| 25 | AVHSLSRIGDELYLE | RAD9>Q99638\|16-30 |
| 26 | NPKFLKNAGRDCSRR | TGM2>P21980\|200-214 |

In some embodiments, the BH3 domain may include an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to any one of SEQ ID NOs: 27-30. In some embodiments, the BH3 domain includes the amino acid sequence of any one of SEQ ID NOs: 27-30. In some embodiments, the BH3 domain includes the amino acid sequence of SEQ ID NO: 27. In some embodiments, the BH3 domain includes the amino acid sequence of SEQ ID NO: 28. Illustrative BH3 domains that may be used according to the present invention are described in Stadler et al, Cell Death and Disease (2014) 5, e1037, 1-9, which is hereby incorporated by reference in its entirety. The BH3 domain of Puma has the amino acid sequence: EEQWAREIGAQLRRMADDLNAQYERR (SEQ ID NO: 27); the BH3 domain of Bim has the amino acid sequence: DMRPEIWIAQELRRIGDEFNAYYARR (SEQ ID NO: 28); the BH3 domain of Bad has the amino acid sequence: NLWAAQRYGRELRRMSDEFVDSFKKG (SEQ ID NO: 29); and the BH3 domain of Noxa has the amino acid sequence: PAELEVECATQLRRFGKLNFRQKLL (SEQ ID NO: 30).

In some embodiments, the BH3 domain-encoding mRNA encodes an amino acid sequence selected from the group consisting of SEQ ID NOs: 196, 197, 207, 225, 234, 236, 237, 291 and 294. In some embodiments, the BH3 domain-encoding mRNA comprises any one of SEQ ID NOs: 225, 244, 245, 246, 273, 281, 283, 284, 290 and 293.

In another embodiment, the mRNA construct encodes one or more truncated BID polypeptides that retain the Bcl-2 homology 3 (BH3) domain. The truncated BID polypeptide containing a BH3 domain contains fewer amino acid residues than a full-length BID protein but still contain the BH3 domain. For example, in one embodiment, the truncated BID (tBID) polypeptide containing its BH3 domain consists of amino acids 61-195 of the BID protein. In another embodiment, the truncated BID (tBID) polypeptide containing its BH3 domain consists of amino acids 77-195 of the BID protein. Non-limiting examples of tBID constructs, and representative sequences thereof, are described further in Example 4.

In some embodiments, a BH3 domain may be able to induce apoptosis. A person of ordinary skill in the art can readily determine if a BH3 domain is able to induce apoptosis using a variety of methods, for example, caspase activation assays (e.g., caspase-3/7 activation assays), stains and dyes (e.g., CELLTOX™, MITOTRACKER® Red, propidium iodide, and YOYO3), cell viability assays, cell morphology, and PARP-1 cleavage.

### TOPK-Inhibitory Peptides

In various embodiments, an mRNA of the disclosure encodes one or more inhibitory peptides of T-lymphokine-activated killer cell-originated protein kinase (TOPK). TOPK is critical for mitosis of breast cancer cells (see, e.g, Matsuo et al., Science Translation Medicine, 6(259): 259ra145, and U.S. Pat. No. 8,673,548). Consequently, TOPK-inhibitory peptides of the invention can be used to treat or prevent cancer.

In particular embodiments, an mRNA of the disclosure encodes a scaffold polypeptide for presenting the TOPK-inhibitory peptides. In other embodiments, an mRNA of the disclosure does not encode a scaffold polypeptide; rather, expression of the one or more TOPK-inhibitory peptides intracellularly is sufficient for their function. In particular embodiments, the isolated mRNAs encode multiple TOPK-inhibitory peptides, referred to herein as multimer or tandem constructs. In one embodiment, the mRNA encodes at least three TOPK-inhibitory peptides. In one embodiment, the mRNA encodes two to ten TOPK-inhibitory peptides. In one embodiment, the mRNA encodes three TOPK-inhibitory peptides. In other embodiments, the mRNA encodes 2, 4, 5, 6, 7, 8, 9 or 10 TOPK-inhibitory peptides. The mRNA may encode a linker as described herein.

When a construct contains multiple TOPK-inhibitory peptides, the construct can contain multiple copies of the same TOPK-inhibitory peptide or, alternatively, can contain a combination of two or more different TOPK-inhibitory peptides. In certain embodiments, the TOPK-inhibitory peptide is selected from SEQ ID NOs: 372-378.

In some embodiments, a TOPK-inhibitory peptide as used herein may include an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to any one of SEQ ID NOs: 376-378. In some embodiments, a peptide or tandem construct may have any one of the following sequences in Table or in the Sequence Listing but for having at least 1, 2, or 3 substitutions, C-terminal or N-terminal additions or deletions as compared to said sequences.

In some embodiments, the TOPK-inhibitory peptide includes an amino acid sequence selected from the group consisting of SEQ ID NOs: 376-378, as shown in Table 2, which also indicates the name and amino acid residues.

TABLE 2
Illustrative TOPK-inhibitory peptides and Tandem Constructs

| SEQ | | |
|---|---|---|
| ID NO | Sequence | Description |
| 372 | TCAAGCTTTTGGACCCTCGTACAGAAGCTAATACGACTCAC | TandemPep. |
| | TATAGGGAAATAAGAGAGAAAAGAAGAGTAAGAAGAAATA | TOPK_ |
| | TAAGAGCCACCATGATGGAAGGAATCAGCAACTTCAAGAC | codon |
| | CCCATCCAAGCTGTCCGAGAAGAAAAAGGGTTCCGGAGTG | optimized |
| | AAGCAGACCCTGAACTTCGATCTGCTCAAGCTCGCCGGGGA | (5′ UTR, |
| | CGTGGAAAGCAACCCTGGTCCCATGGAGGGCATCTCGAACT | ORF, 3′ |
| | TTAAGACCCCCTCGAAGCTTTCGGAGAAGAAGAAGGGATCC | UTR) |
| | GGCGTCAAGCAGACTCTGAATTTCGACTTGCTGAAGCTCGC | |
| | GGGCGATGTGGAATCAAACCCGGGGCCTATGGAAGGCATCT | |
| | CCAACTTCAAAACTCCGTCCAAGCTGAGCGAGAAAAAGAA | |
| | GGGAAAGCCCATTCCGAACCCTCTGCTGGGACTGGACAGCA | |
| | CCTGATAATAGGCTGGAGCCTCGGTGGCCATGCTTCTTGCC | |
| | CCTTGGGCCTCCCCCCAGCCCCTCCTCCCCTTCCTGCACCCG | |
| | TACCCCCGTGGTCTTTGAATAAAGTCTGAGTGGGCGGC | |
| 373 | TCAAGCTTTTGGACCCTCGTACAGAAGCTAATACGACTCAC | TandemPep. |
| | TATAGGGAAATAAGAGAGAAAAGAAGAGTAAGAAGAAATA | TOPK |
| | TAAGAGCCACCATGATGGAAGGCATCAGCAACTTCAAGACC | (5′ UTR, |
| | CCAAGCAAGCTGAGCGAGAAGAAGAAGGGCTCCGGCGTGA | ORF, 3′ |
| | AGCAGACCTTGAACTTCGACCTGCTCAAACTTGCCGGCGAC | UTR) |
| | GTGGAGAGCAACCCCGGCCCCATGGAGGGGATCAGTAACTT | |
| | CAAGACCCCCAGCAAGCTGAGCGAGAAGAAGAAGGGTAGC | |
| | GGCGTGAAACAGACCCTGAATTTCGATCTGCTGAAGCTGGC | |
| | CGGCGACGTGGAGAGCAACCCCGGCCCCATGGAGGGCATA | |
| | AGCAATTTCAAGACCCCCAGCAAGCTGAGCGAAAAGAAAA | |
| | AGGGCAAGCCCATTCCCAACCCCCTTCTGGGCCTTGACAGC | |
| | ACCTGATAATAGGCTGGAGCCTCGGTGGCCATGCTTCTTGC | |
| | CCCTTGGGCCTCCCCCCAGCCCCTCCTCCCCTTCCTGCACCC | |
| | GTACCCCCGTGGTCTTTGAATAAAGTCTGAGTGGGCGGC | |
| 374 | ATGATGGAAGGAATCAGCAACTTCAAGACCCCATCCAAGCT | TandemPep. |
| | GTCCGAGAAGAAAAAGGGTTCCGGAGTGAAGCAGACCCTG | TOPK_ |
| | AACTTCGATCTGCTCAAGCTCGCCGGGGACGTGGAAAGCAA | codon |
| | CCCTGGTCCCATGGAGGGCATCTCGAACTTTAAGACCCCCT | optimized |
| | CGAAGCTTTCGGAGAAGAAGAAGGGATCCGGCGTCAAGCA | (ORF) |
| | GACTCTGAATTTCGACTTGCTGAAGCTCGCGGGCGATGTGG | |
| | AATCAAACCCGGGGCCTATGGAAGGCATCTCCAACTTCAAA | |
| | ACTCCGTCCAAGCTGAGCGAGAAAAAGAAGGGAAAGCCCA | |
| | TTCCGAACCCTCTGCTGGGACTGGACAGCACC | |
| 375 | ATGATGGAAGGCATCAGCAACTTCAAGACCCCAAGCAAGCT | TandemPep. |
| | GAGCGAGAAGAAGAAGGGCTCCGGCGTGAAGCAGACCTTG | TOPK |
| | AACTTCGACCTGCTCAAACTTGCCGGCGACGTGGAGAGCAA | (ORF) |
| | CCCCGGCCCCATGGAGGGGATCAGTAACTTCAAGACCCCCA | |
| | GCAAGCTGAGCGAGAAGAAGAAGGGTAGCGGCGTGAAACA | |
| | GACCCTGAATTTCGATCTGCTGAAGCTGGCCGGCGACGTGG | |
| | AGAGCAACCCCGGCCCCATGGAGGGCATAAGCAATTTCAA | |
| | GACCCCCAGCAAGCTGAGCGAAAAGAAAAAGGGCAAGCCC | |
| | ATTCCCAACCCCCTTCTGGGCCTTGACAGCACC | |
| 376 | MEGISNFKTPSKLSEKKK | Isolated |
| | | TOPK |
| | | inhibitory |
| | | peptide |
| 377 | MMEGISNFKTPSKLSEKKKGSGVKQTLNFDLLKLAGDVESNP | TOPK |
| | GPMEGISNFKTPSKLSEKKKGSGVKQTLNFDLLKLAGDVESNP | inhibitory |
| | GPMEGISNFKTPSKLSEKKKGKPIPNPLLGLDST | 3-peptide |
| | | tandem with |
| | | F2A linker |
| 378 | MMEGISNFKTPSKLSEKKKGSGATNFSLLKQAGDVEENPGPM | TOPK |
| | EGISNFKTPSKLSEKKKGSGATNFSLLKQAGDVEENPGPMEGIS | inhibitory |
| | NFKTPSKLSEKKKGKPIPNPLLGLDST | 3-peptide |
| | | tandem with |
| | | P2A linker |

In some embodiments, a TOPK-inhibitory peptide may be able to inhibit cell proliferation. A person of ordinary skill in the art can readily determine if a TOPK-inhibitory peptide is able to inhibit cell proliferation using a variety of methods known in the art.

### SALL4-Inhibitory Peptides

In various embodiments, an mRNA of the disclosure encodes one or more SALL4-inhibitory peptides. SALL4 encodes a zinc-finger transcription factor that is not normally expressed in adult tissue but is expressed in a subset of hepatocellular carcinomas (WO2013043128; Yong, New Engl. J. Med. 368:2266-2276, 2013). Consequently, SALL4-inhibitory peptides of the disclosure may be used to treat or prevent cancer.

In particular embodiments, an mRNA of the disclosure encodes a scaffold polypeptide for presenting the SALL4-inhibitory peptides. In other embodiments, an mRNA of the disclosure does not encode a scaffold polypeptide; rather, expression of the one or more SALL4-inhibitory peptides intracellularly is sufficient for their function. In particular embodiments, the isolated mRNAs encode multiple SALL4-inhibitory peptides, referred to herein as multimer or tandem constructs. In one embodiment, the mRNA encodes at least three SALL4-inhibitory peptides. In one embodiment, the mRNA encodes two to ten SALL4-inhibitory peptides. In one embodiment, the mRNA encodes three SALL4-inhibitory peptides. In other embodiments, the mRNA encodes 2, 4, 5, 6, 7, 8, 9 or 10 SALL4-inhibitory peptides. The mRNA may encode a linker as described herein.

When a construct contains multiple SALL4-inhibitory peptides, the construct can contain multiple copies of the same SALL4-inhibitory peptide or, alternatively, can contain a combination of two or more different SALL4-inhibitory peptides. In certain embodiments, the SALL4-inhibitory peptide is selected from SEQ ID NOs: 379-385.

In some embodiments, a SALL4-inhibitory peptide as used herein may include an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to any one of SEQ ID NOs: 383-385. In some embodiments, a peptide or tandem construct may have any one of the following sequences in Table 3 or in the Sequence Listing but for having at least 1, 2, or 3 substitutions, C-terminal or N-terminal additions or deletions as compared to said sequences.

In some embodiments, the SALL4-inhibitory peptide includes an amino acid sequence selected from the group consisting of SEQ ID NOs: 383-385, as shown in Table 3, which also indicates the name and amino acid residues.

TABLE 3
Illustrative SALL4-inhibitory Peptides and Tandem Constructs

| SEQ ID | | |
|---|---|---|
| NO | Sequence | Description |
| 379 | TCAAGCTTTTGGACCCTCGTACAGAAGCTAATACGAC | TandemPep.Sall4, |
| | TCACTATAGGGAAATAAGAGAGAAAAGAAGAGTAAG | (5′ UTR, ORF, 3′ |
| | AAGAAATATAAGAGCCACCATGATGAGCAGACGTAA | UTR) |
| | GCAGGCTAAGCCCCAGCATATCGGCAGCGGCGTGAA | |
| | GCAGACCCTGAACTTCGACCTGCTCAAGCTGGCCGGC | |
| | GATGTCGAGTCAAACCCCGGCCCCATGAGCAGAAGA | |
| | AAGCAGGCCAAGCCCCAGCACATCGGTAGCGGAGTG | |
| | AAACAGACCCTGAACTTCGACTTACTGAAGCTCGCTG | |
| | GCGACGTGGAGAGCAACCCCGGCCCCATGAGCAGAA | |
| | GAAAGCAGGCCAAGCCCCAGCACATCGGAAAGCCCA | |
| | TCCCCAACCCCCTGCTGGGCCTGGACAGCACCTGATA | |
| | ATAGGCTGGAGCCTCGGTGGCCATGCTTCTTGCCCCT | |
| | TGGGCCTCCCCCCAGCCCCTCCTCCCCTTCCTGCACCC | |
| | GTACCCCCGTGGTCTTTGAATAAAGTCTGAGTGGGCG | |
| | GC | |
| 380 | TCAAGCTTTTGGACCCTCGTACAGAAGCTAATACGAC | TandemPep.Sall4 |
| | TCACTATAGGGAAATAAGAGAGAAAAGAAGAGTAAG | codon optimized |
| | AAGAAATATAAGAGCCACCATGATGAGCCGCAGAAA | (5′ UTR, ORF, 3′ |
| | GCAGGCCAAGCCTCAGCATATCGGATCCGGCGTGAA | UTR) |
| | GCAGACCCTGAACTTCGACCTTCTGAAGCTGGCCGGC | |
| | GATGTGGAATCCAACCCGGGGCCCATGTCCCGGAGG | |
| | AAACAAGCGAAGCCACAGCACATCGGATCGGGAGTG | |
| | AAGCAAACTCTCAACTTCGACTTGCTGAAACTCGCCG | |
| | GGGATGTCGAGTCAAATCCCGGCCCTATGAGCCGCC | |
| | GGAAGCAGGCTAAGCCGCAGCACATTGGAAAGCCTA | |
| | TCCCCAACCCGCTGCTGGGTCTGGACAGCACCTGATA | |
| | ATAGGCTGGAGCCTCGGTGGCCATGCTTCTTGCCCCT | |
| | TGGGCCTCCCCCCAGCCCCTCCTCCCCTTCCTGCACCC | |
| | GTACCCCCGTGGTCTTTGAATAAAGTCTGAGTGGGCG | |
| | GC | |
| 381 | ATGATGAGCAGACGTAAGCAGGCTAAGCCCCAGCAT | TandemPep.Sall4 |
| | ATCGGCAGCGGCGTGAAGCAGACCCTGAACTTCGAC | (ORF) |
| | CTGCTCAAGCTGGCCGGCGATGTCGAGTCAAACCCCG | |
| | GCCCCATGAGCAGAAGAAAGCAGGCCAAGCCCCAGC | |
| | ACATCGGTAGCGGAGTGAAACAGACCCTGAACTTCG | |
| | ACTTACTGAAGCTCGCTGGCGACGTGGAGAGCAACC | |
| | CCGGCCCCATGAGCAGAAGAAAGCAGGCCAAGCCCC | |
| | AGCACATCGGAAAGCCCATCCCCAACCCCCTGCTGG | |
| | GCCTGGACAGCACC | |
| 382 | ATGATGAGCCGCAGAAAGCAGGCCAAGCCTCAGCAT | TandemPep.Sall4 |
| | ATCGGATCCGGCGTGAAGCAGACCCTGAACTTCGAC | codon optimized |
| | CTTCTGAAGCTGGCCGGCGATGTGGAATCCAACCCGG | (ORF) |
| | GGCCCATGTCCCGGAGGAAACAAGCGAAGCCACAGC | |
| | ACATCGGATCGGGAGTGAAGCAAACTCTCAACTTCG | |
| | ACTTGCTGAAACTCGCCGGGGATGTCGAGTCAAATCC | |
| | CGGCCCTATGAGCCGCCGGAAGCAGGCTAAGCCGCA | |
| | GCACATTGGAAAGCCTATCCCCAACCCGCTGCTGGGT | |
| | CTGGACAGCACC | |
| 383 | MSRRKQAKPQHI | isolated SALL4- |
| | | inhibitory peptide |
| 384 | MMSRRKQAKPQHIGSGVKQTLNFDLLKLAGDVESNPG | SALL4-inhibitory |
| | PMSRRKQAKPQHIGSGVKQTLNFDLLKLAGDVESNPGP | 3-peptide tandem |
| | MSRRKQAKPQHIGKPIPNPLLGLDST | with F2A linker |
| 385 | MMSRRKQAKPQHIGSGATNFSLLKQAGDVEENPGPMS | SALL4-inhibitory |
| | RRKQAKPQHIGSGATNFSLLKQAGDVEENPGPMSRRKQ | 3-peptide tandem |
| | AKPQHIGKPIPNPLLGLDST | with P2A linker |

In some embodiments, a SALL4-inhibitory peptide may be able to inhibit cell proliferation. A person of ordinary skill in the art can readily determine if a SALL4-inhibitory peptide is able to inhibit cell proliferation using a variety of methods known in the art.

### Ras Inhibitory Peptides and Constructs

In various embodiments, an mRNA of the disclosure encodes one or more Ras inhibitory peptides or Ras inhibitory peptide constructs (e.g., multimers of Ras inhibitory peptides). It is known in the art that unregulated activity of RAS gene products can cause cancer (see, e.g., Goodsell, D S Oncologist 4: 263-4, 1999). Anti-Ras peptide ligands which bind Ras have been described (see, e.g., Gareiss, P C ChemBioChem 11: 517-522, 2010). Thus, the invention features methods of altering Ras activity to treat or prevent cancer.

In some embodiments, an mRNA of the disclosure encodes a scaffold polypeptide for presenting the Ras inhibitory peptide. In other embodiments, a scaffold polypeptide is not necessary; rather, expression of the one or more Ras inhibitory peptides intracellularly is sufficient for their function, e.g., expression of a multimer of Ras inhibitory peptide.

In particular embodiments, the isolated mRNAs encode multiple Ras inhibitory peptides, referred to herein as a multimer or tandem construct. In one embodiment, the mRNA encodes at least three Ras inhibitory peptides. In one embodiment, the mRNA encodes two to ten Ras inhibitory peptides. In one embodiment, the mRNA encodes three Ras inhibitory peptides. In other embodiments, the mRNA encodes 2, 4, 5, 6, 7, 8, 9 or 10 Ras inhibitory peptides. In some embodiments, the multimer construct encodes one or more linkers as described herein.

When a construct contains multiple Ras inhibitory peptides, the construct can contain multiple copies of the same Ras inhibitory peptide or, alternatively, can contain a combination of two or more different Ras inhibitory peptides. In certain embodiments, the Ras inhibitory peptides are selected from the group consisting of SEQ ID NOs: 386-392.

In some embodiments, a Ras inhibitory peptide or multimer construct as used herein may include an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to any one of SEQ ID NOs: 386-388. In some embodiments, a Ras inhibitory peptide or multimer construct may have any one of the following sequences in Table 4 or in the Sequence Listing but for having at least 1, 2, or 3 substitutions, C-terminal or N-terminal additions or deletions as compared to said sequences.

In some embodiments, the Ras inhibitory peptide includes an amino acid sequence selected from the group consisting of SEQ ID NOs: 386-388, as shown in Table 4, which also indicates the name and amino acid residues.

TABLE 4
Illustrative Ras Inhibitory Peptides
and Tandem Constructs

| SEQ | | |
|---|---|---|
| ID NO | Sequence | Description |
| 386 | HYPWFKARLYPL | RAS |
| | | inhibitory |
| | | peptide |
| 387 | MHYPWFKARLYPLGSGVKQTLNFDLLKLAG | Ras |
| | DVESNPGPHYPWFKARLYPLGSGVKQTLNF | inhibitory |
| | DLLKLAGDVESNPGPHYPWFKARLYPLGKP | peptide |
| | IPNPLLGLDST | tandem-(3x) |
| | | (GSG)F2A |
| | | linker-V5 |
| | | Tag |
| | | (optional) |
| 388 | MHYPWFKARLYPLGSGATNFSLLKQAGDVE | Ras |
| | ENTGPHYPWFKARLYPLGSGATNFSLIKQA | inhibitory |
| | GDVEENPGPHYPWFKARLYPLGKPIPNPLL | peptide |
| | GLDST | tandem-(3x) |
| | | (GSG) |
| | | P2A linker- |
| | | V5 Tag |
| | | (optional) |

* (GSG) residues can be absent or present, e.g., added to the 5′ end of the peptide to improve cleavage efficiency

In some embodiments, a Ras inhibitory peptide or construct may be able to alter cell growth and proliferation. A person of ordinary skill in the art can readily determine if an anti-Ras peptide is able to affect cell growth and proliferation using a variety of methods known in the art.

### p53 Inhibitory Peptides

In various embodiments, an mRNA of the disclosure encodes one or more p53 inhibitory peptides. p53 is a tumor suppressor (see, e.g., Surget S et al., OncoTargets and Therapy 7: 57-68, 2013) implicated is a wide range of proliferative and/or tumorogenic disorders, in particular, cancer. Consequently, p53-inhibitory peptides of the invention can be used to treat or prevent proliferative and/or tumorogenic disorders, in particular, cancer.

In particular embodiments, an mRNA of the disclosure encodes a scaffold polypeptide for presenting the p53-inhibitory peptides. In other embodiments, an mRNA of the disclosure does not encode a scaffold polypeptide; rather, expression of the one or more p53-inhibitory peptides intracellularly is sufficient for their function. In particular embodiments, the isolated mRNAs encode multiple p53-inhibitory peptides, referred to herein as multimer or tandem constructs. In one embodiment, the mRNA encodes at least three p53-inhibitory peptides. In one embodiment, the mRNA encodes two to ten p53-inhibitory peptides. In one embodiment, the mRNA encodes three p53-inhibitory peptides. In other embodiments, the mRNA encodes 2, 4, 5, 6, 7, 8, 9 or 10 p53-inhibitory peptides. The mRNA may encode a linker as described herein.

When a construct contains multiple p53-inhibitory peptides, the construct can contain multiple copies of the same p53-inhibitory peptide or, alternatively, can contain a combination of two or more different p53-inhibitory peptides. In certain embodiments, the p53-inhibitory peptide is selected from SEQ ID NOs: 393-424.

In some embodiments, the p53-inhibitory peptide is a biologically-active portion, isolated from human p53. In exemplary aspects of the invention, the p53-inhibitory peptide (also referred to herein as a p53-inhibitory domain) is obtained from a full-length or naturally-occurring p53 protein or polypeptide, wherein the peptide or domain lacks the full function of p53, e.g., a functionality and/or biological activity attributed to one or more p53 domains/peptides distinct from said inhibitory domain function. In other embodiments, the p53-inhibitory peptide may be from a non-human species, e.g., Caenorhabditis elegans, rodents (e.g., mice and rats), or non-human primates. In some embodiments, a p53-inhibitory peptide as used herein may include an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to any one of SEQ ID NOs: 393-404. In some embodiments, a peptide or multimer construct may have any one of the following sequences in Table 5 or in the Sequence Listing but for having at least 1, 2, or 3 substitutions, C-terminal or N-terminal additions or deletions as compared to said sequences.

In some embodiments, the p53-inhibitory peptide includes an amino acid sequence selected from the group consisting of SEQ ID NOs: 393-404, as shown in Table 5, which also indicates the name and amino acid residues.

TABLE 5
Illustrative p53-inhibitory peptides and
Tandem Constructs

| SEQ | | |
|---|---|---|
| ID NO | p53-inhibitory sequence | Description |
| 393 | METFSDLWKLLPEGSGVKQTLNFDLLKLAGDVESNP | TandemPep.P53 |
| | GPETFSDLWKLLPEGSGVKQTLNFDLLKLAGDVESNP | ORF |
| | GPETFSDLWKLLPEGKPIPNPLLGLDST | |
| 394 | MLTFEHSWAQLTSGSGVKQTLNFDLLKLAGDVESNP | TandemPep.p53.6S |
| | GPLTFEHSWAQLTSGSGVKQTLNFDLLKLAGDVESNP | ORF |
| | GPLTFEHSWAQLTSGKPIPNPLLGLDST | |
| 395 | METFEHWWAQLTSGSGVKQTLNFDLLKLAGDVESNP | TandemPep.p53. |
| | GPETFEHWWAQLTSGSGVKQTLNFDLLKLAGDVESN | P1E6W |
| | PGPETFEHWWAQLTSGKPIPNPLLGLDST | |
| 396 | MLTFEHWWAQLTSGSGVKQTLNFDLLKLAGDVESNP | TandemPep.p53.P6W |
| | GPLTFEHWWAQLTSGSGVKQTLNFDLLKLAGDVESN | ORF |
| | PGPLTFEHWWAQLTSGKPIPNPLLGLDST | |
| 397 | METFEHWWSQLLSGSGVKQTLNFDLLKLAGDVESNP | TandemPep.p53.pDIQ |
| | GPETFEHWWSQLLSGSGVKQTLNFDLLKLAGDVESN | ORF |
| | PGPETFEHWWSQLLSGKPIPNPLLGLDST | |
| 398 | MTSFAEYWNLLSPGSGVKQTLNFDLLKLAGDVESNP | TandemPep.p53.pMI |
| | GPTSFAEYWNLLSPGSGVKQTLNFDLLKLAGDVESNP | ORF |
| | GPTSFAEYWNLLSPGKPIPNPLLGLDST | |
| 399 | TSFAEYWNLLSP | pMI |
| 400 | ETFSDLWKLLPE | p53 |
| 401 | ETFEHWWSQLLS | pDIQ |
| 402 | ETFEHWWAQLTS | p1E6W |
| 403 | LTFEHSWAQLTS | p536S |
| 404 | LTFEHWWAQLTS | P6W |

In some embodiments, a p53-inhibitory peptide may be able to inhibit cell proliferation. A person of ordinary skill in the art can readily determine if a p53-inhibitory peptide is able to inhibit cell proliferation using a variety of methods known in the art.

### PP2A Inhibitory Peptides

In various embodiments, an mRNA of the disclosure encodes one or more PP2A inhibitory peptides. PP2A is a serine/threonine phosphatase that modulates the activity of proteins in several oncogenic signaling cascades (see, e.g., Kurimchak and Graña, Cell Cycle 14:18-30, 2015). Consequently, PP2A-inhibitory peptides of the disclosure can be used to treat or prevent proliferative and/or tumorigenic disorders, in particular, cancer.

In particular embodiments, an mRNA of the disclosure encodes a scaffold polypeptide for presenting the PP2A-inhibitory peptides. In other embodiments, an mRNA of the disclosure does not encode a scaffold polypeptide; rather, expression of the one or more PP2A-inhibitory peptides intracellularly is sufficient for their function. In particular embodiments, the isolated mRNAs encode multiple PP2A-inhibitory peptides, referred to herein as multimer or tandem constructs. In one embodiment, the mRNA encodes at least three PP2A-inhibitory peptides. In one embodiment, the mRNA encodes two to ten PP2A-inhibitory peptides. In one embodiment, the mRNA encodes three PP2A-inhibitory peptides. In other embodiments, the mRNA encodes 2, 4, 5, 6, 7, 8, 9 or 10 PP2A-inhibitory peptides. The mRNA may encode a linker as described herein.

When a construct contains multiple PP2A-inhibitory peptides, the construct can contain multiple copies of the same PP2A-inhibitory peptide or, alternatively, can contain a combination of two or more different PP2A-inhibitory peptides. In certain embodiments, the PP2A-inhibitory peptide is selected from SEQ ID NOs: 425-442.

In some embodiments, the PP2A-inhibitory peptide is a biologically-active portion, isolated from human PP2A. In exemplary aspects of the disclosure, the PP2A-inhibitory peptide (also referred to herein as a PP2A-inhibitory domain) is obtained from a full-length or naturally-occurring PP2A protein or polypeptide, wherein the peptide or domain lacks the full function of PP2A, e.g., a functionality and/or biological activity attributed to one or more PP2A domains/peptides distinct from said inhibitory domain function. In other embodiments, the PP2A-inhibitory peptide may be from a non-human species, e.g., Caenorhabditis elegans, rodents (e.g., mice and rats), or non-human primates. In some embodiments, a PP2A-inhibitory peptide as used herein may include an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to any one of SEQ ID NOs: 425-432. In some embodiments, a peptide or multimer construct may have any one of the following sequences in Table 6 or in the Sequence Listing but for having at least 1, 2, or 3 substitutions, C-terminal or N-terminal additions or deletions as compared to said sequences.

In some embodiments, the PP2A-inhibitory peptide includes an amino acid sequence selected from the group consisting of SEQ ID NOs: 425-432, as shown in Table 6, which also indicates the name and amino acid residues.

TABLE 6
Illustrative PP2A-inhibitory peptides
and Tandem Constructs

| SEQ | | |
|---|---|---|
| ID NO | Sequence | Description |
| 425 | MTPDYFLGSGVKQTLNFDLLKLAGDVES | TandemPep. |
| | NPGPTPDYFLGSGVKQTLNFDLLKLAGD | PP2aB56 |
| | VESNPGPTPDYFLGKPIPNPLLGLDST | alpha |
| 426 | MVKKKKIKREIKIFRGRSRFRGRSRGSG | TandemPep. |
| | VKQTLNFDLLLAGDVESNPGPVKKKKIK | PP2aDP7, |
| | REIKIFRGRSRFRGRSRGSGVKQTLNFD | ORF |
| | LLKLAGDVESNPGPVKKKKIKREIKIFR | |
| | GRSRFRGRSRGKPIPNPLLGLDST | |
| 427 | MRQKRLIRQKRLIRQKRLIGSGVKQTLN | TandemPep. |
| | FDLLKLAGDVESNPGPRQKRLIRQKRLI | PP2aDPT 2 |
| | RQKRLIGSGVKQTLNFDLLKLAGDVESN | |
| | PGPRQKRLIRQKRLIRQKRLIGKPIPNP | |
| | LLGLDST | |
| 428 | MVKKKKIKREIKIPRRPGPTRKHYQPYA | TandemPep. |
| | GSGVKQTLNFDLLKLAGDVESNPGPVKK | PP2aDPT5 |
| | KKIKREIKIPRRPGPTRKHYQPYAGSGV | ORF |
| | KQTLNFDLLKLAGDVESNPGPVKKKKIK | |
| | REIKIPRRPGPTRKHYQPYAGKPIPNPL | |
| | LGLDST | |
| 429 | TPDYFL | PP2aB56alpha |
| 430 | VKKKKIKREIKIFRGRSRFRGRSR | PP2aDP7 |
| 431 | RQKRLIRQKRLIRQKRLI | PP2aDPT2 |
| 432 | VKKKKIKREIKIPRRPGPTRKHYQPYA | PP2aDPT5 |

In some embodiments, a PP2A-inhibitory peptide may be able to inhibit cell proliferation. A person of ordinary skill in the art can readily determine if a PP2A-inhibitory peptide is able to inhibit cell proliferation using a variety of methods known in the art.

### STAT3 Inhibitory Peptides

In various embodiments, an mRNA of the disclosure encodes one or more STAT3 inhibitory peptides. STAT3 is a transcription factor, and alterations in its activity, such as loss of function, gain of function, or constitutive activation, are associated with recurrent infections, disordered bone and tooth development, auto-immune diseases, and various cancers (see, e.g., Levy D E, Loomis C A, The New England Journal of Medicine 357: 1655-1658, 2007; Milner J D et al., Blood 125: 591-9, 2015; Klampfer L Current Cancer Drug Targets 6: 107-121, 2006; Alvarez J V et al., Cancer Research 66: 3162-3168, 2006; Yin W et al., Molecular Cancer 5: 15. doi:10.1186/1476-4598-5-15, 2006; Kusaba T et al., Oncology Reports 15: 1445-51. doi:10.3892/or.15.6.1445, 2006). Consequently, STAT3-inhibitory peptides of the disclosure can be used to treat or prevent proliferative and/or tumorigenic disorders, in particular, cancer, and auto-immune diseases and infection.

In particular embodiments, an mRNA of the disclosure encodes a scaffold polypeptide for presenting the STAT3-inhibitory peptides. In other embodiments, an mRNA of the disclosure does not encode a scaffold polypeptide; rather, expression of the one or more STAT3-inhibitory peptides intracellularly is sufficient for their function. In particular embodiments, the isolated mRNAs encode multiple STAT3-inhibitory peptides, referred to herein as multimer or tandem constructs. In one embodiment, the mRNA encodes at least three STAT3-inhibitory peptides. In one embodiment, the mRNA encodes two to ten STAT3-inhibitory peptides. In one embodiment, the mRNA encodes three STAT3-inhibitory peptides. In other embodiments, the mRNA encodes 2, 4, 5, 6, 7, 8, 9 or 10 STAT3-inhibitory peptides. The mRNA may encode a linker as described herein.

When a construct contains multiple STAT3-inhibitory peptides, the construct can contain multiple copies of the same STAT3-inhibitory peptide or, alternatively, can contain a combination of two or more different STAT3-inhibitory peptides. In certain embodiments, the STAT3-inhibitory peptide is selected from SEQ ID NOs: 443-447.

In some embodiments, the STAT3-inhibitory peptide is a biologically-active portion, isolated from human STAT3. In exemplary aspects of the invention, the STAT3-inhibitory peptide (also referred to herein as a STAT3-inhibitory domain) is obtained from a full-length or naturally-occurring STAT3 protein or polypeptide, wherein the peptide or domain lacks the full function of STAT3, e.g., a functionality and/or biological activity attributed to one or more STAT3 domains/peptides distinct from said inhibitory domain function. In other embodiments, the STAT3-inhibitory peptide may be from a non-human species, e.g., Caenorhabditis elegans, rodents (e.g., mice and rats), or non-human primates. In some embodiments, a STAT3-inhibitory peptide as used herein may include an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to any one of SEQ ID NOs: 443 and 444. In some embodiments, a peptide or tandem construct may have any one of the following sequences in Table 7 or in the Sequence Listing but for having at least 1, 2, or 3 substitutions, C-terminal or N-terminal additions or deletions as compared to said sequences.

In some embodiments, the STAT3-inhibitory peptide includes an amino acid sequence selected from the group consisting of SEQ ID NOs: 443 and 444, as shown in Table 7, which also indicates the name and amino acid residues.

TABLE 7
Illustrative STAT3-inhibitory peptides
and Tandem Constructs

| SEQ | | |
|---|---|---|
| ID NO | Sequence | Description |
| 443 | MPLTAVFWLIYVLAKALVTVCGSGVKQTLNFDL | TandemPep. |
| | LKLAGDVESNPGPPLTAVFWLIYVLAKALVTVC | STAT3.DBD |
| | GSGVKQTLNFDLLKLAGDVESNPGPPLTAVFWL | ORF |
| | IYVLAKALVTVCGKPIPNPLLGLDST | |
| 444 | PLTAVFWLIYVLAKALVTVC | STAT3 |

In some embodiments, a STAT3-inhibitory peptide may be able to inhibit cell proliferation. A person of ordinary skill in the art can readily determine if a STAT3-inhibitory peptide is able to inhibit cell proliferation using a variety of methods known in the art.

### YAP Binding Polypeptides

In various embodiments, an mRNA of the disclosure encodes one or more YAP binding polypeptides, also referred to as YAP inhibitory domains. YAP is a transcription factor regulated by the Hippo pathway, and alterations in its activity, such as gain in function, are associated with various cancers (see, e.g., Yu, et al., Cell. Vol. 163(4):811-28, 2015; Yimlamai, D., et al., J Hepatol. Vol. 63(6):1491-501, 2015). The Hippo pathway controls several cell functions central to tumorigenesis, e.g., cell proliferation and apoptosis, and is deregulated in several human cancers. The main function of the Hippo pathway is to negatively regulate the activity of YAP. When the Hippo pathway is on, YAP is degraded and a VGLL family member (including VGLL1-4) binds to TEAD1-TEAD4, downregulating downstream genes. When the Hippo pathway is off, YAP binds to TEAD1-TEAD4, inducing transcription of downstream genes. Consequently, YAP binding polypeptides of the disclosure can be used to treat or prevent proliferative and/or tumorigenic disorders, in particular, cancer.

In particular embodiments, an mRNA of the disclosure encodes a scaffold polypeptide for presenting YAP inhibitory domains. In other embodiments, an mRNA of the disclosure does not encode a scaffold polypeptide for presenting the YAP inhibitory domain(s); rather, expression of the one or more YAP inhibitory domains intracellularly is sufficient for their function. In particular embodiments, the isolated mRNAs encode multiple YAP inhibitory domains, referred to herein as multimer or tandem constructs. In one embodiment, the mRNA encodes at least three YAP inhibitory domains. In one embodiment, the mRNA encodes two to ten YAP inhibitory domains. In one embodiment, the mRNA encodes three YAP inhibitory domains. In other embodiments, the mRNA encodes 2, 3, 4, 5, 6, 7, 8, 9 or 10 YAP inhibitory domains.

When a construct contains multiple YAP inhibitory domains, the construct can contain multiple copies of the same YAP inhibitory domain or, alternatively, can contain a combination of two or more different YAP inhibitory domains. When YAP is not bound to a transcript via TEAD1-4 binding, to induce target genes, a VGLL family member (including VGLL1-4) is bound. Specifically, VGLL tondu (TDU) domains (TDU1 and TDU2) bind to TEAD4 (Jiao, S., et al. Cancer Cell, Vol. 25(2): 166-80, 2014 Feb. 10). In certain embodiments, the YAP inhibitory domains are selected from the group consisting of VGLL1, VGLL2, VGLL3, or VGLL4, and combinations thereof. In certain embodiments, the YAP inhibitory domain is selected from SEQ ID NOs: 448-544.

In some embodiments, a YAP inhibitory domain contains domains from both VGLL4 and YAP. In some embodiments, the YAP inhibitory domain contains fragments of TEADs binding regions from VGLL4 and YAP. In certain embodiments, the YAP inhibitory domain has a polyGGS linker between the fragments of TEADs binding regions from VGLL4 and YAP. In certain embodiments the YAP inhibitory domain is the Super-TDU as described in Jiao, S., et al. (Cancer Cell, Vol. 25: 166-180 (2014), herein incorporated by reference).

In some embodiments, the YAP inhibitory domain is a human YAP inhibitory domain. In other embodiments, the YAP inhibitory domain may be from a non-human species, e.g., Caenorhabditis elegans, rodents (e.g., mice and rats), or non-human primates. Typically, an YAP inhibitory domain is derived from a VGLL family member. The invention features methods of inducing apoptosis that involve introducing an mRNA encoding one or more YAP inhibitory domains into a cell under conditions permissive for expression of the one or more YAP inhibitory domains. In some embodiments, an YAP inhibitory domain may directly bind to a YAP family member.

In some embodiments, an YAP inhibitory domain as used herein may include an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to any one of SEQ ID NOs: 448-462. In some embodiments, a peptide or multimer construct may have any one of the following sequences in Table 8 or in the Sequence Listing but for having at least 1, 2, or 3 substitutions, C-terminal or N-terminal additions or deletions as compared to said sequences.

In some embodiments, the mRNA that selectively inhibits YAP encodes an amino acid sequence selected from the group consisting of: SEQ ID NOs: 481, 483, 488, 490 and 498. In some embodiments, the mRNA that selectively inhibits YAP comprises any one of SEQ ID NOs: 508, 510, 515, 517, 518 and 519.

In some embodiments, the YAP inhibitory domain includes an amino acid sequence selected from the group consisting of SEQ ID NOs: 448-462, as shown in Table 8, which also indicates the name and amino acid residues.

TABLE 8
Illustrative YAP Binding Polypeptides and
Scaffold Constructs

| SEQ ID | | Source |
|---|---|---|
| NO | | Protein |
| 448 | SVDDHFAKSLGDTWLQIGGSGNPKTANVPQTVPMRLRKLPDS | Super TDU |
| | FFKPPE | |
| 449 | SVDDHFAKSLGDTWLQIGGSGNPKTANVPQTVPARLRKLPDS | Super TDU |
| | AFKPPE | (MF2A) |
| 450 | SVDDAAAKSLGDTWLQIGGSGNPKTANVPQTVPARLRKLPDS | Super TDU |
| | AFKPPE | (HFMF4A) |
| 451 | DPVVEEHFRRSLGKNYKEPEPAPNSVSITGSVDDHFAKALGD | Human |
| | TWLQIKAAKD | VGLL4- |
| | | TDU 1 and |
| | | 2 |
| 452 | QTLPVASALSSHRTGPPPISPSKRKFSMEPGDEDLDCDNDHVS | Human |
| | KMSRIFNPHLNKTANGDCRRDPRERSRSPIERAVAPTMSLHGS | VGLL4 |
| | HLYTSLPSLGLEQPLALTKNSLDASRPAGLSPTLTPGERQQNR | |
| | PSVITCASAGARNCNLSHCPIAHSGCAAPGPASYRRPPSAATT | |
| | CDPVVEEHFRRSLGKNYKEPEPAPNSVSITGSVDDHFAKALG | |
| | DTWLQIKAAKDGASSSPESASRRGQPASPSAHMVSHSHSPSV | |
| | VS | |
| 453 | MQTLPVASALSSHRTGPPPISPSKRKFSMEPGDEDLDCDNDHV | Human |
| | SKMSRIFNPHLNKTANGDCRRDPRERSRSPIERAVAPTMSLHG | VGLL4 |
| | SHLYTSLPSLGLEQPLALTKNSLDASRPAGLSPTLTPGERQQN | (complete) |
| | RPSVITCASAGARNCNLSHCPIAHSGCAAPGPASYRRPPSAAT | |
| | TCDPVVEEHFRRSLGKNYKEPEPAPNSVSITGSVDDHFAKALG | |
| | DTWLQIKAAKDGASSSPESASRRGQPASPSAHMVSHSHSPSV | |
| | VS | |
| 454 | QTLPVASALSSHRTGPPPISPSKRKFSMEPGDEDLDCDNDHVS | Human |
| | KMSRlFNPHLNKTANGDCRRDPRERSRSPIERAVAPTMSLHGS | VGLL4 |
| | HLYTSLPSLGLEQPLALTKNSLDASRPAGLSPTLTPGERQQNR | (HF4A) |
| | PSVITCASAGARNCNLSHCPIAHSGCAAPGPASYRRPPSAATT | |
| | CDPVVEEAARRSLGKNYKEPEPAPNSVSITGSVDDAAAKALG | |
| | DTWLQIKAAKDGASSSPESASRRGQPASPSAHMVSHSHSPSV | |
| | VS | |
| 455 | MQTLPVASALSSHRTGPPPISPSKRKFSMEPGDEDLDCDNDHV | Human |
| | SKMSRIFNPHLNKTANGDCRRDPRERSRSPIERAVAPTMSLHG | VGLL4 |
| | SHLYTSLPSLGLEQPLALTKNSLDASRPAGLSPTLTPGERQQN | (HF4A) |
| | RPSVITCASAGARNCNLSHCPIAHSGCAAPGPASYRRPPSAAT | (complete) |
| | TCDPVVEEAARRSLGKNYKEPEPAPNSVSITGSVDDAAAKAL | |
| | GDTWLQIKAAKDGASSSPESASRRGQPASPSAHMVSHSHSPS | |
| | VVS | |
| 456 | DPVVEEHFRRSLGKNYK | VGLL4 |
| | | TDU 1 (V1) |
| 457 | DPVVEEHFRRSLGKNYKE | VGLL4 |
| | | TDU 1 (V2) |
| 458 | DPVVEEHFRRSLGKNYKEPE | VGLL4 |
| | | TDU 1 (V3) |
| 459 | SVSITGSVDDHFAKALGDTWLQIK | VGLL4 |
| | | TDU 2 (V1) |
| 460 | SVSITGSVDDHFAKALGDTWLQIKA | VGLL4 |
| | | TDU 2 (V2) |
| 461 | SVSITGSVDDHFAKALGDTWLQIKAAKD | VGLL4 |
| | | TDU 2 (V3) |
| 462 | SVDDHFAKSLGDTWLQI | VGL44 |
| | | Super TDU |

In some embodiments, an YAP inhibitory domain can induce apoptosis. A person of ordinary skill in the art can readily determine if an YAP inhibitory domain is able to induce apoptosis using a variety of methods, for example, caspase activation assays (e.g., caspase-3/7 activation assays), stains and dyes (e.g., CELLTOX™, MITOTRACKER® Red, propidium iodide, and YOYO3), cell viability assays, cell morphology, and PARP-1 cleavage.

### Linkers and Cleavable Peptides

In certain embodiments, the mRNAs of the disclosure encode more than one intracellular binding domain (e.g., BH3 domain), referred to herein as multimer constructs. In certain embodiments of the multimer constructs, the mRNA further encodes a linker located between each domain. The linker can be, for example, a cleavable linker or protease-sensitive linker. In certain embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In certain embodiments, the linker is an F2A linker. In certain embodiments, the linker is a GGGS linker. In certain embodiments, the multimer construct contains three domains with intervening linkers, having the structure: domain-linker-domain-linker-domain e.g., BH3 domain-linker-BH3 domain-linker-BH3 domain.

In one embodiment, the cleavable linker is an F2A linker (e.g., having the amino acid sequence shown in SEQ ID NO: 138). In other embodiments, the cleavable linker is a T2A linker (e.g., having the amino acid sequence shown in SEQ ID NO: 139), a P2A linker (e.g., having the amino acid sequence shown in SEQ ID NO: 140) or an E2A linker (e.g., having the amino acid sequence shown in SEQ ID NO: 141). The skilled artisan will appreciate that other art-recognized linkers may be suitable for use in the constructs of the invention (e.g., encoded by the polynucleotides of the invention). The skilled artisan will likewise appreciate that other multicistronic constructs may be suitable for use in the invention. In exemplary embodiments, the construct design yields approximately equimolar amounts of intrabody and/or domain thereof encoded by the constructs of the invention.

In one embodiment, the self-cleaving peptide may be, but is not limited to, a 2A peptide. A variety of 2A peptides are known and available in the art and may be used, including e.g., the foot and mouth disease virus (FMDV) 2A peptide, the equine rhinitis A virus 2A peptide, the Thosea asigna virus 2A peptide, and the porcine teschovirus-1 2A peptide. 2A peptides are used by several viruses to generate two proteins from one transcript by ribosome-skipping, such that a normal peptide bond is impaired at the 2A peptide sequence, resulting in two discontinuous proteins being produced from one translation event. As a non-limiting example, the 2A peptide may have the protein sequence: GSGATNFSLLKQAGDVEENPGP (SEQ ID NO: 35), fragments or variants thereof. In one embodiment, the 2A peptide cleaves between the last glycine and last proline. As another non-limiting example, the polynucleotides of the present invention may include a polynucleotide sequence encoding the 2A peptide having the protein sequence GSGATNFSLLKQAGDVEENPGP (SEQ ID NO: 35) fragments or variants thereof. One example of a polynucleotide sequence encoding the 2A peptide is: GGAAGCGGAGCTACTAACTTCAGCCTGCTGAAGCAGGCTGGAGACGTGGAGGAG AACCCTGGACCT (SEQ ID NO: 36). In one illustrative embodiment, a 2A peptide is encoded by the following sequence: 5′-TCCGGACTCAGATCCGGGGATCTCAAAATTGTCGCTCCTGTCAAACAAACTCTTA ACTTTGATTTACTCAAACTGGCTGGGGATGTAGAAAGCAATCCAGGTCCACTC-3′(SEQ ID NO: 37). The polynucleotide sequence of the 2A peptide may be modified or codon optimized by the methods described herein and/or are known in the art.

In one embodiment, this sequence may be used to separate the coding regions of two or more polypeptides of interest. As a non-limiting example, the sequence encoding the F2A peptide may be between a first coding region A and a second coding region B (A-F2Apep-B). The presence of the F2A peptide results in the cleavage of the one long protein between the glycine and the proline at the end of the F2A peptide sequence (NPGP is cleaved to result in NPG and P) thus creating separate protein A (with 21 amino acids of the F2A peptide attached, ending with NPG) and separate protein B (with 1 amino acid, P, of the F2A peptide attached). Likewise, for other 2A peptides (P2A, T2A and E2A), the presence of the peptide in a long protein results in cleavage between the glycine and proline at the end of the 2A peptide sequence (NPGP is cleaved to result in NPG and P). Protein A and protein B may be the same or different peptides or polypeptides of interest. In particular embodiments, protein A and protein B are a BH3 domain(s), and a Bcl-2-like polypeptide, in either order. In certain embodiments, the first coding region and the second coding region encode a BH3 domain(s) and a Bcl-2-like polypeptide, in either order.

mRNA

The disclosure provides isolated mRNAs, for example, mRNAs that encode one or more BH3 domains, as well as mRNAs that encode a Bcl-2-like polypeptide or a variant or fragment thereof. In some embodiments, an isolated mRNA of the invention encodes on or more intracellular binding domains described herein. In certain embodiments, an isolated mRNA of the invention encodes both one or more BH3 domains, and the Bcl-2-like polypeptide or variant or fragment thereof.

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. A non-limiting list of possible cap species includes m⁷GpppG, m⁷Gpppm⁷G, m⁷3′dGpppG, m₂^{7,O3′}GpppG, m₂^{7,O3′}GppppG, m₂^{7,O2′}GppppG, m⁷Gpppm⁷G, m⁷3′dGpppG, m₂^{7,O3′}GpppG, m₂^{7,O3′}GppppG, and m₂^{7,O2′}GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

Modified mRNAs

In some embodiments, an mRNA of the invention comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxyuridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (memo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(im⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵₂U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethylcytidine (m⁵Cm), N4-acetyl-2′-O-methyl-cytidine (ac⁴Cm), N4,2′-O-dimethylcytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (mt⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosylqueuosine (manQ), 7-cyano-7-deaza-guanosine (preQ₀), 7-aminomethyl-7-deaza-guanosine (preQ₁), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m^{2,7}Gm), 2′-O-methyl-inosine (Im), 1,2′-O-dimethylinosine (m¹Im), 2′-O-ribosylguanosine (phosphate) (Gr(p)), 1-thio-guanosine, 06-methyl-guanosine, 2′-F-ara-guanosine, and 2′-F-guanosine.

In some embodiments, an mRNA of the invention includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the invention includes a combination of one or more of the aforementioned modified nucleobases (e.g., a combination of 2, 3 or 4 of the aforementioned modified nucleobases.) In some embodiments, the modified nucleobase is a modified cytosine.

Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²C), 2-thio-5-methyl-cytidine. In some embodiments, an mRNA of the invention includes a combination of one or more of the aforementioned modified nucleobases (e.g., a combination of 2, 3 or 4 of the aforementioned modified nucleobases.) In some embodiments, the modified nucleobase is a modified adenine.

Exemplary nucleobases and nucleosides having a modified adenine include 7-deaza-adenine, 1-methyl-adenosine (m¹A), 2-methyl-adenine (m²A), N6-methyl-adenosine (m⁶A). In some embodiments, an mRNA of the invention includes a combination of one or more of the aforementioned modified nucleobases (e.g., a combination of 2, 3 or 4 of the aforementioned modified nucleobases.) In some embodiments, the modified nucleobase is a modified guanine.

Exemplary nucleobases and nucleosides having a modified guanine include inosine (I), 1-methyl-inosine (m¹I), wyosine (imG), methylwyosine (mimG), 7-deaza-guanosine, 7-cyano-7-deaza-guanosine (preQ₀), 7-aminomethyl-7-deaza-guanosine (preQ₁), 7-methyl-guanosine (m⁷G), 1-methyl-guanosine (m¹G), 8-oxo-guanosine, 7-methyl-8-oxo-guanosine. In some embodiments, an mRNA of the invention includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m¹ψ), 5-methoxy-uridine (mo⁵U), 5-methyl-cytidine (m⁵C), pseudouridine (ψ), α-thio-guanosine, or α-thio-adenosine. In some embodiments, an mRNA of the invention includes a combination of one or more of the aforementioned modified nucleobases (e.g., a combination of 2, 3 or 4 of the aforementioned modified nucleobases.)

In some embodiments, the mRNA comprises pseudouridine (ψ). In some embodiments, the mRNA comprises pseudouridine (ψ) and 5-methyl-cytidine (m⁵C). In some embodiments, the mRNA comprises 1-methyl-pseudouridine (m¹ψ). In some embodiments, the mRNA comprises 1-methyl-pseudouridine (m¹ψ) and 5-methyl-cytidine (m⁵C). In some embodiments, the mRNA comprises 2-thiouridine (s²U). In some embodiments, the mRNA comprises 2-thiouridine and 5-methyl-cytidine (m⁵C). In some embodiments, the mRNA comprises 5-methoxy-uridine (mo⁵U). In some embodiments, the mRNA comprises 5-methoxy-uridine (mo⁵U) and 5-methyl-cytidine (m⁵C). In some embodiments, the mRNA comprises 2′-O-methyl uridine. In some embodiments, the mRNA comprises 2′-O-methyl uridine and 5-methyl-cytidine (m⁵C). In some embodiments, the mRNA comprises comprises N6-methyl-adenosine (m⁶A). In some embodiments, the mRNA comprises N6-methyl-adenosine (m⁶A) and 5-methyl-cytidine (m⁵C).

In certain embodiments, an mRNA of the invention is uniformly modified (i.e., fully modified, modified through-out the entire sequence) for a particular modification. For example, an mRNA can be uniformly modified with 5-methyl-cytidine (m⁵C), meaning that all cytosine residues in the mRNA sequence are replaced with 5-methyl-cytidine (m⁵C). Similarly, mRNAs of the invention can be uniformly modified for any type of nucleoside residue present in the sequence by replacement with a modified residue such as those set forth above.

In some embodiments, an mRNA of the invention may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present invention include, but are not limited to, those described in PCT Patent Application Publications: WO2012045075, WO2014081507, WO2014093924, WO2014164253, and WO2014159813.

The mmRNAs of the invention can include a combination of modifications to the sugar, the nucleobase, and/or the internucleoside linkage. These combinations can include any one or more modifications described herein.

Examples of modified nucleosides and modified nucleoside combinations are provided below in Table 9 and Table 10. These combinations of modified nucleotides can be used to form the mmRNAs of the invention. In certain embodiments, the modified nucleosides may be partially or completely substituted for the natural nucleotides of the mRNAs of the invention. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

TABLE 9
Combinations of Nucleoside Modifications

| Modified Nucleotide | Modified Nucleotide Combination |
|---|---|
| α-thio-cytidine | α-thio-cytidine/5-iodo-uridine |
| | α-thio-cytidine/N1-methyl-pseudouridine |
| | α-thio-cytidine/α-thio-uridine |
| | α-thio-cytidine/5-methyl-uridine |
| | α-thio-cytidine/pseudo-uridine |
| | about 50% of the cytosines are α-thio-cytidine |
| pseudoisocytidine | pseudoisocytidine/5-iodo-uridine |
| | pseudoisocytidine/N1-methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% of uridines are N1- |
| | methyl-pseudouridine and about 50% of uridines |
| | are pseudouridine |
| | pseudoisocytidine/about 25% of uridines are N1- |
| | methyl-pseudouridine and about 25% of uridines |
| | are pseudouridine |
| pyrrolo-cytidine | pyrrolo-cytidine/5-iodo-uridine |
| | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl-cytidine | 5-methyl-cytidine/5-iodo-uridine |
| | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines are 2- |
| | thio-uridine |
| | about 50% of uridines are 5-methyl-cytidine/about |
| | 50% of uridines are 2-thio-uridine |
| N4-acetyl-cytidine | N4-acetyl-cytidine/5-iodo-uridine |
| | N4-acetyl-cytidine/N1-methyl-pseudouridine |
| | N4-acetyl-cytidine/α-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/ |
| | about 50% of uridines are 2-thio-uridine |

TABLE 10
Modified Nucleosides and Combinations Thereof

1-(2,2,2-Trifluoroethyl)pseudo-UTP
1-Ethyl-pseudo-UTP
1-Methyl-pseudo-U-alpha-thio-TP
1-methyl-pseudouridine TP, ATP, GTP, CTP
1-methyl-pseudo-UTP/5-methyl-CTP/ATP/GTP
1-methyl-pseudo-UTP/CTP/ATP/GTP
1-Propyl-pseudo-UTP
25% 5-Aminoallyl-CTP + 75% CTP/25% 5-Methoxy-UTP + 75% UTP
25% 5-Aminoallyl-CTP + 75% CTP/75% 5-Methoxy-UTP + 25% UTP
25% 5-Bromo-CTP + 75% CTP/25% 5-Methoxy-UTP + 75% UTP
25% 5-Bromo-CTP + 75% CTP/75% 5-Methoxy-UTP + 25% UTP
25% 5-Bromo-CTP + 75% CTP/1-Methyl-pseudo-UTP
25% 5-Carboxy-CTP + 75% CTP/25% 5-Methoxy-UTP + 75% UTP
25% 5-Carboxy-CTP + 75% CTP/75% 5-Methoxy-UTP + 25% UTP
25% 5-Ethyl-CTP + 75% CTP/25% 5-Methoxy-UTP + 75% UTP
25% 5-Ethyl-CTP + 75% CTP/75% 5-Methoxy-UTP + 25% UTP
25% 5-Ethynyl-CTP + 75% CTP/25% 5-Methoxy-UTP + 75% UTP
25% 5-Ethynyl-CTP + 75% CTP/75% 5-Methoxy-UTP + 25% UTP
25% 5-Fluoro-CTP + 75% CTP/25% 5-Methoxy-UTP + 75% UTP
25% 5-Fluoro-CTP + 75% CTP/75% 5-Methoxy-UTP + 25% UTP
25% 5-Formyl-CTP + 75% CTP/25% 5-Methoxy-UTP + 75% UTP
25% 5-Formyl-CTP + 75% CTP/75% 5-Methoxy-UTP + 25% UTP
25% 5-Hydroxymethyl-CTP + 75% CTP/25% 5-Methoxy-UTP +
75% UTP
25% 5-Hydroxymethyl-CTP + 75% CTP/75% 5-Methoxy-UTP +
25% UTP
25% 5-Iodo-CTP + 75% CTP/25% 5-Methoxy-UTP + 75% UTP
25% 5-Iodo-CTP + 75% CTP/75% 5-Methoxy-UTP + 25% UTP
25% 5-Methoxy-CTP + 75% CTP/25% 5-Methoxy-UTP + 75% UTP
25% 5-Methoxy-CTP + 75% CTP/75% 5-Methoxy-UTP + 25% UTP
25% 5-Methyl-CTP + 75% CTP/25% 5-Methoxy-UTP + 75%
1-Methyl-pseudo-UTP
25% 5-Methyl-CTP + 75% CTP/25% 5-Methoxy-UTP + 75% UTP
25% 5-Methyl-CTP + 75% CTP/50% 5-Methoxy-UTP + 50%
1-Methyl-pseudo-UTP
25% 5-Methyl-CTP + 75% CTP/50% 5-Methoxy-UTP + 50% UTP
25% 5-Methyl-CTP + 75% CTP/5-Methoxy-UTP
25% 5-Methyl-CTP + 75% CTP/75% 5-Methoxy-UTP + 25%
1-Methyl-pseudo-UTP
25% 5-Methyl-CTP + 75% CTP/75% 5-Methoxy-UTP + 25% UTP
25% 5-Phenyl-CTP + 75% CTP/25% 5-Methoxy-UTP + 75% UTP
25% 5-Phenyl-CTP + 75% CTP/75% 5-Methoxy-UTP + 25% UTP
25% 5-Trifluoromethyl-CTP + 75% CTP/25% 5-Methoxy-UTP +
75% UTP
25% 5-Trifluoromethyl-CTP + 75% CTP/75% 5-Methoxy-UTP +
25% UTP
25% 5-Trifluoromethyl-CTP + 75% CTP/1-Methyl-pseudo-UTP
25% N4-Ac-CTP + 75% CTP/25% 5-Methoxy-UTP + 75% UTP
25% N4-Ac-CTP + 75% CTP/75% 5-Methoxy-UTP + 25% UTP
25% N4-Bz-CTP + 75% CTP/25% 5-Methoxy-UTP + 75% UTP
25% N4-Bz-CTP + 75% CTP/75% 5-Methoxy-UTP + 25% UTP
25% N4-Methyl-CTP + 75% CTP/25% 5-Methoxy-UTP + 75% UTP
25% N4-Methyl-CTP + 75% CTP/75% 5-Methoxy-UTP + 25% UTP
25% Pseudo-iso-CTP + 75% CTP/25% 5-Methoxy-UTP + 75% UTP
25% Pseudo-iso-CTP + 75% CTP/75% 5-Methoxy-UTP + 25% UTP
25% 5-Bromo-CTP/75% CTP/Pseudo-UTP
25% 5-methoxy-UTP/25% 5-methyl-CTP/ATP/GTP
25% 5-methoxy-UTP/5-methyl-CTP/ATP/GTP
25% 5-methoxy-UTP/75% 5-methyl-CTP/ATP/GTP
25% 5-methoxy-UTP/CTP/ATP/GTP
25% 5-metoxy-UTP/50% 5-methyl-CTP/ATP/GTP
2-Amino-ATP
2-Thio-CTP
2-thio-pseudouridine TP, ATP, GTP, CTP
2-Thio-pseudo-UTP
2-Thio-UTP
3-Methyl-CTP
3-Methyl-pseudo-UTP
4-Thio-UTP
50% 5-Bromo-CTP + 50% CTP/1-Methyl-pseudo-UTP
50% 5-Hydroxymethyl-CTP + 50% CTP/1-Methyl-pseudo-UTP
50% 5-methoxy-UTP/5-methyl-CTP/ATP/GTP
50% 5-Methyl-CTP + 50% CTP/25% 5-Methoxy-UTP + 75%
1-Methyl-pseudo-UTP
50% 5-Methyl-CTP + 50% CTP/25% 5-Methoxy-UTP + 75% UTP
50% 5-Methyl-CTP + 50% CTP/50% 5-Methoxy-UTP + 50%
1-Methyl-pseudo-UTP
50% 5-Methyl-CTP + 50% CTP/50% 5-Methoxy-UTP + 50% UTP
50% 5-Methyl-CTP + 50% CTP/5-Methoxy-UTP
50% 5-Methyl-CTP + 50% CTP/75% 5-Methoxy-UTP + 25%
1-Methyl-pseudo-UTP
50% 5-Methyl-CTP + 50% CTP/75% 5-Methoxy-UTP + 25% UTP
50% 5-Trifluoromethyl-CTP + 50% CTP/1-Methyl-pseudo-UTP
50% 5-Bromo-CTP/50% CTP/Pseudo-UTP
50% 5-methoxy-UTP/25% 5-methyl-CTP/ATP/GTP
50% 5-methoxy-UTP/50% 5-methyl-CTP/ATP/GTP
50% 5-methoxy-UTP/75% 5-methyl-CTP/ATP/GTP
50% 5-methoxy-UTP/CTP/ATP/GTP
5-Aminoallyl-CTP
5-Aminoallyl-CTP/5-Methoxy-UTP
5-Aminoallyl-UTP
5-Bromo-CTP
5-Bromo-CTP/5-Methoxy-UTP
5-Bromo-CTP/1-Methyl-pseudo-UTP
5-Bromo-CTP/Pseudo-UTP
5-bromocytidine TP, ATP, GTP, UTP
5-Bromo-UTP
5-Carboxy-CTP/5-Methoxy-UTP
5-Ethyl-CTP/5-Methoxy-UTP
5-Ethynyl-CTP/5-Methoxy-UTP
5-Fluoro-CTP/5-Methoxy-UTP
5-Formyl-CTP/5-Methoxy-UTP
5-Hydroxy-methyl-CTP/5-Methoxy-UTP
5-Hydroxymethyl-CTP
5-Hydroxymethyl-CTP/1-Methyl-pseudo-UTP
5-Hydroxymethyl-CTP/5-Methoxy-UTP
5-hydroxymethyl-cytidine TP, ATP, GTP, UTP
5-Iodo-CTP/5-Methoxy-UTP
5-Me-CTP/5-Methoxy-UTP
5-Methoxy carbonyl methyl-UTP
5-Methoxy-CTP/5-Methoxy-UTP
5-methoxy-uridine TP, ATP, GTP, UTP
5-methoxy-UTP
5-Methoxy-UTP
5-Methoxy-UTP/N6-Isopentenyl-ATP
5-methoxy-UTP/25% 5-methyl-CTP/ATP/GTP
5-methoxy-UTP/5-methyl-CTP/ATP/GTP
5-methoxy-UTP/75% 5-methyl-CTP/ATP/GTP
5-methoxy-UTP/CTP/ATP/GTP
5-Methyl-2-thio-UTP
5-Methylaminomethyl-UTP
5-Methyl-CTP/5-Methoxy-UTP
5-Methyl-CTP/5-Methoxy-UTP(cap 0)
5-Methyl-CTP/5-Methoxy-UTP(No cap)
5-Methyl-CTP/25% 5-Methoxy-UTP + 75% 1-Methyl-pseudo-UTP
5-Methyl-CTP/25% 5-Methoxy-UTP + 75% UTP
5-Methyl-CTP/50% 5-Methoxy-UTP + 50% 1-Methyl-pseudo-UTP
5-Methyl-CTP/50% 5-Methoxy-UTP + 50% UTP
5-Methyl-CTP/5-Methoxy-UTP/N6-Me-ATP
5-Methyl-CTP/75% 5-Methoxy-UTP + 25% 1-Methyl-pseudo-UTP
5-Methyl-CTP/75% 5-Methoxy-UTP + 25% UTP
5-Phenyl-CTP/5-Methoxy-UTP
5-Trifluoro-methyl-CTP/5-Methoxy-UTP
5-Trifluoromethyl-CTP
5-Trifluoromethyl-CTP/5-Methoxy-UTP
5-Trifluoromethyl-CTP/1-Methyl-pseudo-UTP
5-Trifluoromethyl-CTP/Pseudo-UTP
5-Trifluoromethyl-UTP
5-trifluromethylcytidine TP, ATP, GTP, UTP
75% 5-Aminoallyl-CTP + 25% CTP/25% 5-Methoxy-UTP + 75% UTP
75% 5-Aminoallyl-CTP + 25% CTP/75% 5-Methoxy-UTP + 25% UTP
75% 5-Bromo-CTP + 25% CTP/25% 5-Methoxy-UTP + 75% UTP
75% 5-Bromo-CTP + 25% CTP/75% 5-Methoxy-UTP + 25% UTP
75% 5-Carboxy-CTP + 25% CTP/25% 5-Methoxy-UTP + 75% UTP
75% 5-Carboxy-CTP + 25% CTP/75% 5-Methoxy-UTP + 25% UTP
75% 5-Ethyl-CTP + 25% CTP/25% 5-Methoxy-UTP + 75% UTP
75% 5-Ethyl-CTP + 25% CTP/75% 5-Methoxy-UTP + 25% UTP
75% 5-Ethynyl-CTP + 25% CTP/25% 5-Methoxy-UTP + 75% UTP
75% 5-Ethynyl-CTP + 25% CTP/75% 5-Methoxy-UTP + 25% UTP
75% 5-Fluoro-CTP + 25% CTP/25% 5-Methoxy-UTP + 75% UTP
75% 5-Fluoro-CTP + 25% CTP/75% 5-Methoxy-UTP + 25% UTP
75% 5-Formyl-CTP + 25% CTP/25% 5-Methoxy-UTP + 75% UTP
75% 5-Formyl-CTP + 25% CTP/75% 5-Methoxy-UTP + 25% UTP
75% 5-Hydroxymethyl-CTP + 25% CTP/25% 5-Methoxy-UTP +
75% UTP
75% 5-Hydroxymethyl-CTP + 25% CTP/75% 5-Methoxy-UTP +
25% UTP
75% 5-Iodo-CTP + 25% CTP/25% 5-Methoxy-UTP + 75% UTP
75% 5-Iodo-CTP + 25% CTP/75% 5-Methoxy-UTP + 25% UTP
75% 5-Methoxy-CTP + 25% CTP/25% 5-Methoxy-UTP + 75% UTP
75% 5-Methoxy-CTP + 25% CTP/75% 5-Methoxy-UTP + 25% UTP
75% 5-methoxy-UTP/5-methyl-CTP/ATP/GTP
75% 5-Methyl-CTP + 25% CTP/25% 5-Methoxy-UTP + 75%
1-Methyl-pseudo-UTP
75% 5-Methyl-CTP + 25% CTP/25% 5-Methoxy-UTP + 75% UTP
75% 5-Methyl-CTP + 25% CTP/50% 5-Methoxy-UTP + 50%
1-Methyl-pseudo-UTP
75% 5-Methyl-CTP + 25% CTP/50% 5-Methoxy-UTP + 50% UTP
75% 5-Methyl-CTP + 25% CTP/5-Methoxy-UTP
75% 5-Methyl-CTP + 25% CTP/75% 5-Methoxy-UTP + 25%
1-Methyl-pseudo-UTP
75% 5-Methyl-CTP + 25% CTP/75% 5-Methoxy-UTP + 25% UTP
75% 5-Phenyl-CTP + 25% CTP/25% 5-Methoxy-UTP + 75% UTP
75% 5-Phenyl-CTP + 25% CTP/75% 5-Methoxy-UTP + 25% UTP
75% 5-Trifluoromethyl-CTP + 25% CTP/25% 5-Methoxy-UTP +
75% UTP
75% 5-Trifluoromethyl-CTP + 25% CTP/75% 5-Methoxy-UTP +
25% UTP
75% 5-Trifluoromethyl-CTP + 25% CTP/1-Methyl-pseudo-UTP
75% N4-Ac-CTP + 25% CTP/25% 5-Methoxy-UTP + 75% UTP
75% N4-Ac-CTP + 25% CTP/75% 5-Methoxy-UTP + 25% UTP
75% N4-Bz-CTP + 25% CTP/25% 5-Methoxy-UTP + 75% UTP
75% N4-Bz-CTP + 25% CTP/75% 5-Methoxy-UTP + 25% UTP
75% N4-Methyl-CTP + 25% CTP/25% 5-Methoxy-UTP + 75% UTP
75% N4-Methyl-CTP + 25% CTP/75% 5-Methoxy-UTP + 25% UTP
75% Pseudo-iso-CTP + 25% CTP/25% 5-Methoxy-UTP + 75% UTP
75% Pseudo-iso-CTP + 25% CTP/75% 5-Methoxy-UTP + 25% UTP
75% 5-Bromo-CTP/25% CTP/1-Methyl-pseudo-UTP
75% 5-Bromo-CTP/25% CTP/Pseudo-UTP
75% 5-methoxy-UTP/25% 5-methyl-CTP/ATP/GTP
75% 5-methoxy-UTP/50% 5-methyl-CTP/ATP/GTP
75% 5-methoxy-UTP/75% 5-methyl-CTP/ATP/GTP
75% 5-methoxy-UTP/CTP/ATP/GTP
8-Aza-ATP
Alpha-thio-CTP
CTP/25% 5-Methoxy-UTP + 75% 1-Methyl-pseudo-UTP
CTP/25% 5-Methoxy-UTP + 75% UTP
CTP/50% 5-Methoxy-UTP + 50% 1-Methyl-pseudo-UTP
CTP/50% 5-Methoxy-UTP + 50% UTP
CTP/5-Methoxy-UTP
CTP/5-Methoxy-UTP (cap 0)
CTP/5-Methoxy-UTP(No cap)
CTP/75% 5-Methoxy-UTP + 25% 1-Methyl-pseudo-UTP
CTP/75% 5-Methoxy-UTP + 25% UTP
CTP/UTP(No cap)
N1-Me-GTP
N4-Ac-CTP
N4Ac-CTP/1-Methyl-pseudo-UTP
N4Ac-CTP/5-Methoxy-UTP
N4-acetyl-cytidine TP, ATP, GTP, UTP
N4-Bz-CTP/5-Methoxy-UTP
N4-methyl CTP
N4-Methyl-CTP/5-Methoxy-UTP
Pseudo-iso-CTP/5-Methoxy-UTP
PseudoU-alpha-thio-TP
pseudouridine TP, ATP, GTP, CTP
pseudo-UTP/5-methyl-CTP/ATP/GTP
UTP-5-oxyacetic acid Me ester
Xanthosine

According to the invention, polynucleotides of the invention may be synthesized to comprise the combinations or single modifications of Table 9 or Table 10.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

The mRNAs of the present invention, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, Calif.) and/or proprietary methods. In one embodiments, the mRNA sequence is optimized using optimization algorithms, e.g., to optimize expression in mammalian cells or enhance mRNA stability.

In certain embodiments, the present invention includes polynucleotides having at least 80%, at least 85%, at least 90%, at least 95%, at least 98%, or at least 99% sequence identity to any of the polynucleotide sequences described herein. mRNAs of the present invention may be produced by means available in the art, including but not limited to in vitro transcription (IVT) and synthetic methods.

Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present invention also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

### Sequence Optimization of Nucleotide Sequence Encoding a BH3 Domain Polypeptide

In some embodiments, the polynucleotide (e.g., a RNA, e.g., a mRNA) of the invention is sequence optimized. In some embodiments, the polynucleotide (e.g., a RNA, e.g., a mRNA) of the invention comprises a nucleotide sequence (e.g., an ORF) encoding one or more BH3 domains, a 5′-UTR, a 3′-UTR, a miRNA, a nucleotide sequence encoding a linker, or any combination thereof, that is sequence optimized.

A sequence optimized nucleotide sequence, e.g., a codon optimized mRNA sequence encoding a BH3 polypeptide (e.g., a BH3 multimeric polypeptide, e.g., PUMA BH3 multimer), is a sequence comprising at least one synonymous nucleobase substitution with respect to a reference sequence (e.g., a wild type nucleotide sequence encoding a BH3 polypeptide).

A sequence optimized nucleotide sequence can be partially or completely different in sequence from the reference sequence. For example, a reference sequence encoding polyserine uniformly encoded by TCT codons can be sequence optimized by having 100% of its nucleobases substituted (for each codon, T in position 1 replaced by A, C in position 2 replaced by G, and T in position 3 replaced by C) to yield a sequence encoding polyserine which would be uniformly encoded by AGC codons. The percentage of sequence identity obtained from a global pairwise alignment between the reference polyserine nucleic acid sequence and the sequence optimized polyserine nucleic acid sequence would be 0%. However, the protein products from both sequences would be 100% identical.

Some sequence optimization (also sometimes referred to codon optimization) methods are known in the art (and discussed in more detail below) and can be useful to achieve one or more desired results. These results can include, e.g., matching codon frequencies in certain tissue targets and/or host organisms to ensure proper folding; biasing G/C content to increase mRNA stability or reduce secondary structures; minimizing tandem repeat codons or base runs that can impair gene construction or expression; customizing transcriptional and translational control regions; inserting or removing protein trafficking sequences; removing/adding post translation modification sites in an encoded protein (e.g., glycosylation sites); adding, removing or shuffling protein domains; inserting or deleting restriction sites; modifying ribosome binding sites and mRNA degradation sites; adjusting translational rates to allow the various domains of the protein to fold properly; and/or reducing or eliminating problem secondary structures within the polynucleotide. Sequence optimization tools, algorithms and services are known in the art, non-limiting examples include services from GeneArt (Life Technologies), DNA2.0 (Menlo Park Calif.) and/or proprietary methods.

Codon options for each amino acid are given in Table 11.

TABLE 11
Codon Options

| | Single Letter | |
|---|---|---|
| Amino Acid | Code | Codon Options |
| Isoleucine | I | ATT, ATC, ATA |
| Leucine | L | CTT, CTC, CTA, CTG, TTA, TTG |
| Valine | V | GTT, GTC, GTA, GTG |
| Phenylalanine | F | TTT, TTC |
| Methionine | M | ATG |
| Cysteine | C | TGT, TGC |
| Alanine | A | GCT, GCC, GCA, GCG |
| Glycine | G | GGT, GGC, GGA, GGG |
| Proline | P | CCT, CCC, CCA, CCG |
| Threonine | T | ACT, ACC, ACA, ACG |
| Serine | S | TCT, TCC, TCA, TCG, AGT, AGC |
| Tyrosine | Y | TAT, TAC |
| Tryptophan | W | TGG |
| Glutamine | Q | CAA, CAG |
| Asparagine | N | AAT, AAC |
| Histidine | H | CAT, CAC |
| Glutamic acid | E | GAA, GAG |
| Aspartic acid | D | GAT, GAC |
| Lysine | K | AAA, AAG |
| Arginine | R | CGT, CGC, CGA, CGG, AGA, AGG |
| Selenocysteine | Sec | UGA in mRNA in presence of |
| | | Selenocysteine insertion element |
| | | (SECIS) |
| Stop codons | Stop | TAA, TAG, TGA |

In some embodiments, a polynucleotide (e.g., a RNA, e.g., a mRNA) of the invention comprises a sequence-optimized nucleotide sequence (e.g., an ORF) encoding a polypeptide (e.g., BH3 polypeptide), a functional fragment, or a variant thereof, wherein the polypeptide (e.g., BH3 polypeptide), functional fragment, or a variant thereof encoded by the sequence-optimized nucleotide sequence has improved properties (e.g., compared to a BH3 polypeptide, functional fragment, or a variant thereof encoded by a reference nucleotide sequence that is not sequence optimized), e.g., improved properties related to expression efficacy after administration in vivo. Such properties include, but are not limited to, improving nucleic acid stability (e.g., mRNA stability), increasing translation efficacy in the target tissue, reducing the number of truncated proteins expressed, improving the folding or prevent misfolding of the expressed proteins, reducing toxicity of the expressed products, reducing cell death caused by the expressed products, increasing and/or decreasing protein aggregation.

In some embodiments, the sequence optimized nucleotide sequence is codon optimized for expression in human subjects, having structural and/or chemical features that avoid one or more of the problems in the art, for example, features which are useful for optimizing formulation and delivery of nucleic acid-based therapeutics while retaining structural and functional integrity; overcoming a threshold of expression; improving expression rates; half-life and/or protein concentrations; optimizing protein localization; and avoiding deleterious bio-responses such as the immune response and/or degradation pathways.

In some embodiments, the polynucleotides of the invention comprise a nucleotide sequence (e.g., a nucleotide sequence (e.g, an ORF) encoding a BH3 polypeptide, a nucleotide sequence (e.g, an ORF) encoding another polypeptide of interest, a 5′-UTR, a 3′-UTR, a microRNA, a nucleic acid sequence encoding a linker, or any combination thereof) that is sequence-optimized according to a method comprising:
(i) substituting at least one codon in a reference nucleotide sequence (e.g., an ORF encoding a BH3 polypeptide) with an alternative codon to increase or decrease uridine content to generate a uridine-modified sequence;
(ii) substituting at least one codon in a reference nucleotide sequence (e.g., an ORF encoding a BH3 polypeptide) with an alternative codon having a higher codon frequency in the synonymous codon set;
(iii) substituting at least one codon in a reference nucleotide sequence (e.g., an ORF encoding a BH3 polypeptide) with an alternative codon to increase G/C content; or
(iv) a combination thereof.

In some embodiments, the sequence optimized nucleotide sequence (e.g., an ORF encoding a BH3 polypeptide) has at least one improved property with respect to the reference nucleotide sequence.

In some embodiments, the sequence optimization method is multiparametric and comprises one, two, three, four, or more methods disclosed herein and/or other optimization methods known in the art.

Features, which can be considered beneficial in some embodiments of the invention, can be encoded by or within regions of the polynucleotide and such regions can be upstream (5′) to, downstream (3′) to, or within the region that encodes the BH3 polypeptide. These regions can be incorporated into the polynucleotide before and/or after sequence-optimization of the protein encoding region or open reading frame (ORF). Examples of such features include, but are not limited to, untranslated regions (UTRs), microRNA sequences, Kozak sequences, oligo(dT) sequences, poly-A tail, and detectable tags and can include multiple cloning sites that can have XbaI recognition.

In some embodiments, the polynucleotide of the invention comprises a 5′ UTR, a 3′ UTR and/or a miRNA. In some embodiments, the polynucleotide comprises two or more 5′ UTRs and/or 3′ UTRs, which can be the same or different sequences. In some embodiments, the polynucleotide comprises two or more miRNA, which can be the same or different sequences. Any portion of the 5′ UTR, 3′ UTR, and/or miRNA, including none, can be sequence-optimized and can independently contain one or more different structural or chemical modifications, before and/or after sequence optimization.

In some embodiments, after optimization, the polynucleotide is reconstituted and transformed into a vector such as, but not limited to, plasmids, viruses, cosmids, and artificial chromosomes. For example, the optimized polynucleotide can be reconstituted and transformed into chemically competent E. coli, yeast, neurospora, maize, drosophila, etc. where high copy plasmid-like or chromosome structures occur by methods described herein.

### Sequence-Optimized Nucleotide Sequences Encoding One or More BH3 Domains

In some embodiments, the polynucleotide of the invention comprises a sequence optimized nucleotide sequence encoding a polypeptide disclosed herein (e.g., BH3 polypeptide). In some embodiments, the polynucleotide of the invention comprises an open reading frame (ORF) encoding a multimeric polypeptide (e.g., PUMA BH3 multimeric polypeptide), wherein the ORF has been sequence optimized.

Exemplary sequence optimized nucleotide sequences encoding PUMA-BH3 multimer are shown in Table 12. In some embodiments, the sequence optimized PUMA-BH3 multimer sequences in Table 12, fragments, and variants thereof are used to practice the methods disclosed herein. In some embodiments, the sequence optimized PUMA-BH3 multimer sequences in Table 12, fragments and variants thereof are combined with or alternatives to the wild-type sequences disclosed herein.

TABLE 12
Sequence optimized sequences for PUMA-BH3 Multimers

| SEQ | |
|---|---|
| ID | |
| NO: | SEQUENCE |
| 300 | ATGGAGGAGCAGTGGGCGCGGGAGATAGGGGCCCAGCTAAGGCGGATGG |
| | CCGACGACCTAAACGCCCAATACGAAAGGAGGGGCTCGGGGGTCAAACA |
| | GACCCTCAATTTCGACCTCCTCAAGCTCGCGGGAGACGTCGAGAGCAACC |
| | CCGGCCCCGAGGAGCAGTGGGCGCGCGAAATAGGGGCCCAGCTCCGGCG |
| | CATGGCCGACGACCTCAACGCGCAATACGAGAGGCGCGGCAGCGGGGTA |
| | AAGCAAACGTTGAACTTCGACCTCCTCAAGCTCGCAGGGGACGTGGAGTC |
| | CAACCCCGGGCCCGAAGAACAATGGGCCCGGGAAATCGGCGCCCAGCTG |
| | CGCCGTATGGCTGACGACCTCAACGCGCAGTATGAACGCCGGGGGAAGC |
| | CCATCCCCAACCCCCTGCTCGGCCTCGATAGCACG |
| 301 | ATGGAGGAGCAGTGGGCCCGCGAGATAGGCGCCCAGCTCCGTAGGATGG |
| | CGGACGATCTAAACGCCCAGTACGAGAGGCGGGGCAGCGGGGTCAAACA |
| | GACATTGAATTTCGACCTCTTGAAGCTCGCCGGCGACGTGGAGAGCAACC |
| | CCGGGCCCGAGGAGCAGTGGGCGCGGGAGATCGGAGCCCAACTCAGGAG |
| | AATGGCCGACGACCTCAACGCCCAGTACGAGCGACGCGGTAGCGGGGTA |
| | AAGCAAACCCTCAACTTCGACCTCCTCAAGCTCGCCGGGGACGTTGAGTC |
| | CAATCCCGGGCCCGAAGAACAGTGGGCCCGGGAGATAGGCGCCCAGCTG |
| | CGTCGTATGGCCGACGATCTGAACGCCCAGTACGAGCGGAGAGGGAAGC |
| | CCATCCCGAACCCGTTGCTGGGGCTGGACAGCACC |
| 302 | ATGGAGGAACAGTGGGCCCGCGAGATCGGCGCCCAACTCCGGCGGATGG |
| | CCGACGATCTCAACGCCCAGTACGAGAGGCGGGGCTCCGGGGTTAAGCA |
| | AACCCTCAATTTCGACCTCCTCAAGCTTGCCGGGGACGTCGAAAGTAACC |
| | CCGGCCCGGAGGAACAGTGGGCCCGGGAGATAGGGGCGCAGCTACGCAG |
| | GATGGCCGACGATCTCAACGCCCAGTACGAGAGGAGGGGGTCGGGGGTC |
| | AAGCAGACCCTCAACTTCGACCTACTCAAGCTCGCCGGCGACGTGGAGAG |
| | CAACCCCGGGCCCGAAGAACAGTGGGCCCGGGAGATCGGGGCCCAGCTG |
| | AGACGGATGGCCGATGACCTGAACGCTCAGTACGAGCGGCGTGGGAAGC |
| | CCATCCCCAACCCCCTGCTGGGTTTAGACAGCACC |
| 303 | ATGGAGGAACAGTGGGCCCGCGAGATCGGCGCCCAGCTCCGCCGCATGG |
| | CGGACGATCTTAACGCCCAATACGAGAGGAGGGGGTCCGGCGTCAAGCA |
| | GACCCTCAACTTCGACCTCCTCAAACTCGCCGGAGACGTCGAGTCCAACC |
| | CCGGTCCCGAAGAACAGTGGGCCCGGGAAATCGGGGCCCAGCTCCGCCG |
| | CATGGCAGACGATCTCAACGCCCAGTACGAGCGGCGCGGGTCCGGGGTC |
| | AAGCAGACTCTCAACTTCGATCTTCTCAAGTTAGCGGGGGACGTGGAGTC |
| | CAATCCAGGTCCGGAGGAGCAGTGGGCCCGGGAGATAGGGGCCCAGCTC |
| | CGCCGAATGGCCGACGACCTGAACGCTCAATATGAGCGCCGGGGGAAAC |
| | CCATCCCCAACCCGCTGCTCGGGCTGGATAGCACT |
| 304 | ATGGAGGAGCAGTGGGCAAGGGAGATAGGAGCTCAGCTCAGGCGGATGG |
| | CCGACGACCTCAACGCGCAGTACGAACGGCGGGGATCCGGAGTCAAACA |
| | GACATTGAATTTCGACCTTCTCAAACTCGCCGGCGACGTTGAGAGCAATC |
| | CCGGGCCCGAGGAACAGTGGGCGCGGGAAATCGGCGCCCAGCTAAGGCG |
| | GATGGCCGACGACCTAAACGCCCAATACGAGCGGCGGGGGTCCGGCGTG |
| | AAGCAGACCCTAAACTTCGACCTCCTGAAGCTTGCCGGGGACGTGGAGAG |
| | CAATCCCGGCCCCGAAGAACAGTGGGCCCGGGAGATCGGGGCCCAGCTG |
| | CGGCGCATGGCTGACGACCTCAACGCCCAGTACGAGCGGCGGGGGAAGC |
| | CCATCCCCAACCCGCTCCTGGGTCTGGACAGCACA |
| 305 | ATGGAGGAACAGTGGGCCAGGGAAATCGGGGCCCAGCTAAGGAGGATGG |
| | CCGACGACCTAAACGCCCAGTACGAACGGCGAGGTAGCGGGGTCAAGCA |
| | GACTCTCAACTTCGACCTCTTGAAACTCGCCGGGGACGTCGAGTCGAATC |
| | CAGGCCCCGAGGAGCAGTGGGCACGAGAAATAGGGGCCCAGCTACGCCG |
| | CATGGCGGACGACCTCAACGCTCAATACGAGCGAAGAGGATCCGGCGTA |
| | AAACAGACGTTGAACTTCGACCTCCTCAAGCTCGCCGGGGACGTAGAGTC |
| | CAATCCGGGCCCTGAGGAACAGTGGGCCCGGGAGATCGGGGCCCAGCTG |
| | CGCCGAATGGCGGACGATCTGAATGCCCAGTATGAGAGGAGGGGGAAGC |
| | CCATCCCAAATCCACTGCTGGGTCTGGATTCGACA |
| 306 | ATGGAGGAGCAGTGGGCGCGAGAGATCGGCGCCCAGCTCCGTAGGATGG |
| | CAGACGACTTAAACGCCCAATACGAACGCCGGGGGAGCGGCGTCAAACA |
| | GACGCTCAACTTCGACTTACTAAAACTAGCCGGCGACGTTGAGAGCAATC |
| | CCGGGCCCGAGGAGCAGTGGGCCCGGGAGATAGGCGCGCAGCTTCGCCG |
| | CATGGCGGACGACCTCAACGCCCAATACGAGCGCCGCGGGTCCGGGGTC |
| | AAGCAGACGCTCAACTTCGACCTCCTCAAACTGGCCGGAGACGTGGAGAG |
| | CAACCCCGGCCCCGAGGAGCAGTGGGCCCGCGAAATCGGGGCCCAGCTG |
| | CGCAGAATGGCGGACGACCTGAACGCGCAGTATGAGCGACGGGGGAAGC |
| | CCATCCCGAACCCCCTGCTCGGACTCGACTCCACT |
| 307 | ATGGAGGAGCAGTGGGCCAGGGAGATCGGCGCACAGCTCCGCCGCATGG |
| | CGGACGACCTCAACGCCCAATACGAACGACGGGGGTCCGGGGTCAAACA |
| | GACGCTCAACTTCGACCTCCTTAAACTCGCCGGCGACGTAGAGTCTAACC |
| | CCGGCCCCGAGGAGCAGTGGGCCCGGGAGATAGGGGCCCAGCTCCGGCG |
| | GATGGCCGACGATCTCAACGCCCAGTACGAGCGTAGGGGGAGCGGCGTT |
| | AAGCAAACGCTTAATTTCGACCTCCTCAAGCTCGCGGGCGACGTCGAGTC |
| | AAACCCCGGGCCAGAGGAGCAGTGGGCCCGTGAGATCGGTGCCCAGCTG |
| | AGGCGAATGGCCGATGACCTGAACGCCCAGTATGAGCGCCGTGGGAAGC |
| | CCATTCCGAATCCTCTCCTGGGTCTGGACAGCACC |
| 308 | ATGGAGGAACAGTGGGCTCGCGAGATCGGGGCTCAGCTCCGTAGGATGG |
| | CCGACGATCTCAACGCCCAGTACGAGCGCAGGGGGAGCGGCGTCAAGCA |
| | GACCTTGAATTTCGACCTCCTCAAGCTCGCCGGAGACGTCGAGTCCAACC |
| | CAGGGCCCGAGGAGCAGTGGGCCCGCGAGATCGGAGCCCAGCTCCGGAG |
| | GATGGCAGACGACTTGAACGCACAGTACGAGCGCCGGGGGTCCGGGGTT |
| | AAGCAAACCCTCAACTTCGACCTCCTTAAGCTGGCAGGCGACGTGGAGTC |
| | GAATCCCGGGCCCGAGGAGCAGTGGGCCAGGGAGATCGGCGCACAGCTG |
| | CGGCGCATGGCCGACGACCTGAACGCGCAGTATGAGCGCCGAGGTAAGC |
| | CCATCCCCAACCCCCTGCTTGGGCTGGACTCCACC |
| 309 | ATGGAGGAGCAGTGGGCCCGAGAGATCGGCGCCCAGCTCAGGCGGATGG |
| | CCGACGACCTTAACGCCCAGTACGAGCGGCGGGGGAGCGGGGTCAAGCA |
| | GACCCTTAATTTCGACCTTCTCAAACTGGCCGGGGACGTCGAGTCGAACC |
| | CCGGGCCAGAGGAGCAGTGGGCCAGGGAGATCGGAGCCCAATTACGACG |
| | GATGGCCGACGACCTCAACGCCCAATACGAGCGGAGGGGGTCCGGAGTC |
| | AAACAGACCCTCAACTTCGATCTCTTGAAGCTCGCAGGAGACGTCGAAAG |
| | CAATCCCGGGCCCGAAGAACAGTGGGCCCGGGAGATAGGGGCACAGCTC |
| | CGCAGGATGGCCGACGATCTGAACGCCCAGTACGAGCGTAGGGGTAAAC |
| | CTATCCCAAACCCACTTCTGGGGCTGGACAGCACT |
| 310 | ATGGAAGAACAGTGGGCTCGCGAGATCGGCGCTCAGCTCCGACGGATGG |
| | CCGACGACTTGAACGCGCAGTACGAGCGCCGGGGGAGCGGAGTCAAGCA |
| | GACACTCAACTTCGACCTCCTAAAGTTGGCGGGCGACGTGGAGAGCAACC |
| | CGGGGCCCGAGGAGCAGTGGGCGAGGGAGATAGGCGCCCAGCTGCGCCG |
| | GATGGCCGACGACTTGAACGCTCAATACGAGCGGAGGGGGTCCGGCGTC |
| | AAGCAGACGCTTAATTTCGACCTCCTCAAGCTCGCCGGCGACGTGGAATC |
| | CAACCCCGGCCCGGAGGAGCAGTGGGCCCGAGAAATCGGAGCCCAACTG |
| | CGGAGGATGGCTGACGACCTGAACGCCCAGTACGAGCGCCGAGGAAAGC |
| | CGATCCCCAACCCCCTGCTGGGACTGGACAGCACG |
| 311 | ATGGAGGAGCAGTGGGCCCGGGAAATCGGGGCCCAGTTACGCAGGATGG |
| | CCGACGATCTAAACGCCCAATACGAGAGGAGGGGCTCGGGGGTAAAACA |
| | GACCCTCAATTTCGATTTGCTCAAACTCGCCGGCGACGTCGAGAGTAACC |
| | CGGGCCCCGAGGAGCAGTGGGCCCGCGAGATCGGGGCGCAGCTCCGGCG |
| | GATGGCAGACGACCTCAACGCGCAGTACGAACGCCGGGGCTCCGGCGTC |
| | AAGCAAACGTTGAACTTCGACCTCCTCAAACTCGCCGGGGACGTAGAGAG |
| | CAACCCCGGGCCCGAGGAGCAGTGGGCTCGTGAGATTGGCGCCCAGCTAC |
| | GCCGTATGGCCGACGACCTCAACGCCCAGTACGAGAGGAGGGGTAAGCC |
| | GATCCCCAACCCCCTGCTGGGGCTGGACTCCACC |
| 312 | ATGGAGGAACAGTGGGCGCGAGAGATCGGGGCCCAGCTCAGGCGGATGG |
| | CCGACGATCTCAACGCCCAGTACGAACGGAGGGGTAGCGGGGTAAAGCA |
| | AACTCTAAACTTCGATCTCCTCAAGCTCGCCGGCGACGTAGAGTCCAATC |
| | CGGGGCCCGAGGAGCAGTGGGCGCGGGAGATCGGCGCCCAGCTCCGGAG |
| | GATGGCAGACGATCTCAACGCCCAGTACGAGCGGAGAGGCAGCGGGGTC |
| | AAACAGACCCTCAACTTCGATCTCCTAAAGCTCGCCGGGGACGTGGAGAG |
| | CAACCCCGGGCCCGAGGAGCAGTGGGCCCGCGAAATCGGTGCCCAGCTTC |
| | GACGTATGGCCGATGATCTGAACGCCCAATACGAGCGGCGCGGCAAACC |
| | CATTCCCAATCCGCTGCTCGGGCTGGACTCCACC |
| 313 | ATGGAGGAGCAGTGGGCCCGGGAAATCGGAGCCCAACTACGGCGCATGG |
| | CCGACGACCTCAACGCCCAATACGAGCGGAGGGGCTCGGGAGTCAAGCA |
| | GACTCTAAATTTCGACCTCCTCAAGCTCGCGGGCGACGTCGAGTCCAACC |
| | CCGGTCCCGAAGAACAGTGGGCACGAGAGATCGGCGCCCAGCTCCGGCG |
| | AATGGCGGACGACCTTAACGCCCAGTACGAGCGGCGGGGGAGCGGGGTC |
| | AAGCAAACACTCAACTTCGACCTACTCAAGCTCGCCGGGGACGTCGAGAG |
| | CAATCCCGGGCCCGAGGAACAGTGGGCCAGGGAGATTGGGGCCCAGCTG |
| | AGGAGGATGGCGGACGACCTGAACGCCCAGTACGAGAGGCGAGGCAAGC |
| | CGATCCCCAATCCCCTGCTGGGCCTGGATTCCACC |
| 314 | ATGGAGGAGCAGTGGGCGCGCGAGATAGGCGCCCAACTCCGTAGGATGG |
| | CCGACGATCTTAACGCCCAGTACGAGCGCCGGGGTAGCGGGGTGAAGCA |
| | GACCCTCAACTTCGACCTTCTCAAGCTTGCCGGGGACGTAGAAAGCAATC |
| | CCGGGCCCGAGGAGCAGTGGGCCAGGGAAATCGGGGCCCAGCTCCGCCG |
| | TATGGCCGACGACCTCAACGCGCAGTACGAGCGCCGAGGGTCGGGAGTC |
| | AAGCAGACCCTCAACTTCGATCTCCTCAAGCTCGCCGGCGACGTGGAAAG |
| | CAACCCGGGCCCCGAAGAACAGTGGGCCCGGGAGATTGGGGCACAGCTG |
| | AGGAGGATGGCCGACGATCTGAACGCCCAGTACGAACGGCGGGGCAAGC |
| | CCATCCCAAACCCGCTGCTAGGACTGGACTCAACG |
| 315 | ATGGAGGAGCAGTGGGCACGAGAAATCGGCGCCCAGCTTCGTCGGATGG |
| | CCGACGATCTCAACGCGCAGTACGAGAGGCGGGGCTCGGGAGTCAAACA |
| | GACCCTCAACTTCGACCTCCTCAAGCTCGCCGGCGACGTCGAGTCCAACC |
| | CGGGCCCGGAAGAACAGTGGGCCAGAGAGATCGGGGCCCAGCTAAGGCG |
| | TATGGCCGACGATCTCAACGCCCAGTACGAGCGGAGGGGCTCCGGCGTCA |
| | AGCAGACCCTTAATTTCGATCTCTTGAAGCTCGCCGGGGACGTCGAAAGC |
| | AATCCCGGGCCCGAGGAACAGTGGGCCCGGGAAATCGGTGCACAGCTCA |
| | GGCGCATGGCGGATGATCTCAACGCCCAATACGAGCGCCGGGGCAAACC |
| | CATACCTAACCCCCTGCTCGGTCTGGACTCCACC |
| 316 | ATGGAGGAGCAGTGGGCGCGGGAGATCGGGGCGCAGCTAAGGAGGATGG |
| | CGGACGATCTCAACGCGCAGTACGAAAGGCGCGGCAGCGGCGTGAAGCA |
| | GACGCTCAACTTCGACCTACTCAAGCTCGCGGGGGACGTCGAATCGAACC |
| | CCGGCCCGGAGGAACAGTGGGCCAGGGAGATCGGCGCCCAGCTACGGCG |
| | TATGGCCGACGACCTCAACGCCCAATACGAGAGGAGGGGGTCGGGAGTC |
| | AAACAGACCCTAAACTTCGACCTCCTCAAGCTCGCCGGGGACGTCGAGTC |
| | CAACCCCGGTCCCGAGGAGCAGTGGGCCAGGGAAATCGGGGCGCAACTG |
| | CGCCGCATGGCCGACGATCTGAACGCCCAGTATGAGCGCAGGGGCAAGC |
| | CGATCCCGAATCCGCTGCTAGGTCTGGACTCCACC |
| 317 | ATGGAGGAGCAGTGGGCCCGCGAGATCGGCGCACAGCTCCGACGAATGG |
| | CCGACGATCTCAACGCCCAATACGAACGGCGGGGGAGCGGAGTCAAGCA |
| | GACTTTAAACTTCGACCTCCTCAAGCTTGCCGGGGACGTGGAAAGTAACC |
| | CCGGACCGGAGGAGCAGTGGGCCCGCGAGATAGGAGCGCAGCTCAGGCG |
| | CATGGCCGACGATCTCAACGCCCAATACGAGCGGAGGGGAAGCGGGGTA |
| | AAACAGACGCTCAACTTCGACCTCCTCAAATTAGCCGGCGACGTGGAGAG |
| | CAACCCCGGGCCCGAGGAGCAGTGGGCCCGCGAGATAGGGGCCCAACTG |
| | CGGCGCATGGCGGACGACCTGAACGCCCAGTACGAGAGGCGGGGCAAGC |
| | CGATCCCTAACCCCCTGCTGGGGTTGGACTCCACC |
| 318 | ATGGAAGAACAGTGGGCACGGGAGATCGGCGCACAGCTAAGGAGGATGG |
| | CCGACGACCTTAACGCGCAGTACGAGCGCAGAGGGAGCGGCGTCAAGCA |
| | GACGCTCAATTTCGACCTTCTCAAGCTCGCGGGGGACGTTGAGTCCAATC |
| | CCGGACCCGAGGAGCAGTGGGCCCGCGAGATAGGGGCCCAGCTCCGGCG |
| | GATGGCAGACGATCTCAACGCCCAATACGAGAGGAGGGGGTCGGGGGTC |
| | AAACAGACCCTTAACTTCGACCTCCTCAAGCTCGCCGGGGACGTCGAGAG |
| | TAACCCCGGACCGGAGGAGCAGTGGGCCCGGGAAATTGGCGCCCAACTC |
| | AGGCGGATGGCCGATGATCTCAACGCCCAGTACGAACGTCGGGGTAAGC |
| | CCATCCCGAACCCCCTGCTGGGGCTGGACTCGACC |
| 319 | ATGGAGGAGCAGTGGGCAAGGGAGATAGGCGCACAGCTCCGTCGGATGG |
| | CCGACGACCTGAACGCCCAATACGAGCGGAGAGGGTCCGGGGTCAAGCA |
| | GACCCTCAATTTCGACTTGCTCAAGCTGGCAGGGGACGTCGAAAGCAACC |
| | CCGGCCCGGAGGAGCAGTGGGCGCGCGAGATCGGCGCCCAGCTTAGGCG |
| | GATGGCCGACGACTTAAACGCGCAATACGAGCGCCGCGGCAGCGGGGTC |
| | AAACAGACCCTAAACTTCGACCTCCTCAAGCTCGCCGGCGACGTGGAGAG |
| | CAACCCCGGCCCCGAAGAACAGTGGGCCCGCGAGATCGGGGCGCAGCTG |
| | CGTAGAATGGCCGACGATCTGAACGCCCAGTATGAGAGGCGGGGCAAAC |
| | CTATCCCGAATCCACTGCTGGGCCTGGACAGCACA |
| 320 | ATGGAGGAACAGTGGGCTCGCGAGATAGGCGCCCAGCTCCGCAGAATGG |
| | CCGACGATCTTAACGCCCAATACGAACGGCGGGGGTCCGGGGTCAAGCA |
| | GACGTTAAACTTCGACCTCCTCAAACTCGCCGGGGACGTGGAGTCCAACC |
| | CCGGGCCCGAGGAGCAGTGGGCGCGCGAGATCGGGGCCCAGCTCCGACG |
| | GATGGCCGACGACCTCAACGCGCAGTACGAGCGCAGAGGAAGCGGGGTC |
| | AAGCAGACCCTCAACTTCGATCTCCTCAAGTTGGCGGGCGACGTTGAAAG |
| | CAACCCCGGACCGGAGGAGCAATGGGCCCGCGAGATCGGGGCCCAACTC |
| | AGGAGGATGGCGGACGACCTGAACGCCCAGTACGAACGGAGGGGGAAAC |
| | CTATCCCCAACCCTCTACTGGGGCTGGACTCTACG |
| 321 | ATGGAGGAACAGTGGGCCCGCGAGATCGGCGCCCAACTCCGTAGGATGG |
| | CCGACGATCTCAACGCCCAGTACGAGAGGAGGGGGAGCGGGGTCAAGCA |
| | GACGCTCAACTTCGACCTCCTCAAGCTCGCCGGGGACGTCGAGTCCAACC |
| | CGGGTCCAGAGGAGCAGTGGGCGAGGGAAATCGGCGCCCAGCTCCGTCG |
| | GATGGCCGACGACCTAAACGCGCAGTACGAGAGGAGGGGTTCCGGCGTT |
| | AAACAAACGCTCAACTTCGACCTCCTCAAACTCGCCGGGGACGTCGAGAG |
| | CAACCCCGGACCCGAGGAGCAGTGGGCTCGGGAGATTGGGGCCCAGCTG |
| | AGGCGGATGGCCGATGACCTGAATGCGCAGTACGAGCGCCGCGGAAAAC |
| | CCATCCCTAACCCGCTGCTCGGCCTGGACTCCACC |
| 322 | ATGGAGGAGCAGTGGGCCCGAGAAATAGGGGCCCAGCTCAGGAGGATGG |
| | CCGACGACCTCAACGCCCAATACGAAAGGAGGGGGTCGGGCGTCAAGCA |
| | GACCCTTAATTTCGACTTGCTTAAGCTTGCCGGGGACGTAGAATCCAACC |
| | CGGGACCCGAGGAGCAGTGGGCCCGAGAAATCGGAGCCCAGCTCCGCCG |
| | AATGGCGGACGATCTCAACGCCCAATACGAGAGGAGGGGATCCGGCGTC |
| | AAGCAGACGCTCAATTTCGACCTCCTCAAACTCGCCGGCGACGTTGAATC |
| | AAACCCGGGGCCGGAAGAACAGTGGGCCAGAGAGATCGGCGCACAGCTG |
| | CGCCGAATGGCCGATGACCTGAACGCCCAGTACGAGCGCCGGGGCAAGC |
| | CCATACCGAACCCCCTCCTGGGCCTGGACTCCACC |
| 323 | ATGGAGGAGCAGTGGGCCCGCGAAATCGGCGCCCAGCTCCGGAGAATGG |
| | CCGACGACCTTAACGCCCAGTACGAAAGGAGGGGCAGCGGGGTCAAACA |
| | GACGCTAAACTTCGACCTCCTCAAGCTCGCCGGGGACGTTGAGTCCAACC |
| | CCGGGCCGGAGGAACAGTGGGCGCGGGAGATCGGGGCGCAGCTTAGGCG |
| | AATGGCCGACGACCTAAACGCCCAGTACGAGCGCAGGGGGTCGGGCGTC |
| | AAGCAGACCCTCAACTTCGACCTCCTTAAACTCGCGGGGGACGTCGAGAG |
| | CAATCCGGGGCCGGAAGAACAGTGGGCTCGGGAGATTGGCGCCCAGCTG |
| | CGGCGCATGGCCGATGACCTGAACGCCCAGTATGAACGCCGCGGTAAGCC |
| | CATCCCGAACCCGCTGCTGGGTCTGGATAGCACC |
| 324 | ATGGAGGAACAGTGGGCCCGGGAGATCGGCGCCCAGCTCAGGCGGATGG |
| | CGGACGACCTCAACGCCCAGTACGAGCGGAGGGGGAGCGGGGTCAAGCA |
| | AACCCTCAATTTCGACCTCCTCAAGTTGGCCGGCGACGTGGAGTCGAACC |
| | CCGGGCCCGAGGAACAGTGGGCCCGCGAGATAGGGGCACAGCTCCGCAG |
| | GATGGCCGACGACCTTAACGCGCAGTACGAGAGGAGGGGCTCGGGAGTT |
| | AAGCAGACCCTCAATTTCGATCTCCTCAAACTAGCCGGGGACGTAGAAAG |
| | CAACCCCGGCCCCGAGGAGCAGTGGGCCCGAGAAATCGGCGCGCAGCTG |
| | AGAAGGATGGCTGACGACCTGAACGCGCAGTATGAGAGACGGGGGAAGC |
| | CGATCCCCAACCCCCTCCTCGGGTTGGACTCCACC |

The sequence optimized nucleotide sequences disclosed herein are distinct from the corresponding wild type nucleotide acid sequences and from other known sequence optimized nucleotide sequences, e.g., these sequence optimized nucleic acids have unique compositional characteristics.

In some embodiments, the percentage of uracil or thymine nucleobases in a sequence optimized nucleotide sequence (e.g., encoding a BH3 polypeptide, a functional fragment, or a variant thereof) is modified (e.g, reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence optimized nucleotide sequence has a lower uracil or thymine content than the uracil or thymine content in the reference wild-type sequence. In some embodiments, the uracil or thymine content in a sequence-optimized nucleotide sequence of the invention is greater than the uracil or thymine content in the reference wild-type sequence and still maintain beneficial effects, e.g., increased expression and/or reduced Toll-Like Receptor (TLR) response when compared to the reference wild-type sequence.

As shown in Table 13B, the uracil or thymine content of wild-type PUMA-BH3 multimer is about 13.75%. In some embodiments, the uracil or thymine content of a uracil- or thymine-modified sequence encoding a PUMA-BH3 multimer polypeptide is less than 13.75%. In some embodiments, the uracil or thymine content of a uracil- or thymine-modified sequence encoding a PUMA-BH3 multimer polypeptide of the invention is less than 19%, less that 18%, less than 17%, less than 16%, less than 15%, less than 14%, less than 13%, less than 12%, less than 11%, or less than 10%. In some embodiments, the uracil or thymine content is not less than 18%, 17%, 16%, 15%, 14%, 13%, 12%, 11%, or 10%. The uracil or thymine content of a sequence disclosed herein, i.e., its total uracil or thymine content is abbreviated herein as % U_{TL} or % T_{TL}.

TABLE 13A

| | |
|---|---|
| PumaBH3 | MEEQWAREIGAQLRRMADDLNAQYERRGSGVKQTLNFD |
| (x3P2A).v5 | LLKLAGDVESNPGPEEQWAREIGAQLRRMADDLNAQYE |
| | RRGSGVKQTLNFDLLKLAGDVESNPGPEEQWAREIGAQ |
| | LRRMADDLNAQYERRGKPIPNPLLGLDST |
| | (SEQ ID NO: 370) |
| PumaBH3 | ATGGAGGAGCAATGGGCTAGAGAGATCGGCGCACAGCT |
| (x3P2A).v5 | GCGGCGCATGGCCGATGATCTGAACGCCCAATACGAGA |
| | GGAGAGGTTCCGGAGTGAAGCAGACTCTGAACTTCGAT |
| | CTGCTCAAGCTTGCGGGCGACGTGGAATCGAACCCCGG |
| | CCCTGAGGAACAATGGGCGCGCGAAATCGGTGCCCAGC |
| | TCCGCCGGATGGCAGACGACCTGAACGCGCAGTACGAG |
| | CGGCGGGGGAGCGGGGTCAAGCAGACCCTGAATTTCGA |
| | CCTTCTGAAGCTGGCCGGAGATGTGGAGTCAAACCCGG |
| | GACCCGAAGAACAGTGGGCCAGGGAAATTGGAGCTCAG |
| | CTGCGGAGAATGGCCGACGACCTCAACGCCCAGTACGA |
| | ACGGCGCGGAAAACCTATCCCGAACCCACTCTTGGGCC |
| | TGGACTCCACC (SEQ ID NO: 371) |

TABLE 13B

| | | | Th. Min. | Th. Min. U |
|---|---|---|---|---|
| Protein | | Length | U (%) | (abs) |
| PumaBH3 | MEEQWA | 143 | 9.09 | 39 |
| (x3P2A).v5 | | | | |

| | | | Th. Max. | Th. Max. |
|---|---|---|---|---|
| Protein | | Length | G (%) | G (abs) |
| PumaBH3 | MEEQWA | 143 | 47.09 | 202 |
| (x3P2A).v5 | | | | |
| | | | Th. Max. | Th. Max. |
| Protein | | Length | C (%) | C (abs) |
| PumaBH3 | MEEQWA | 143 | 43.12 | 185 |
| (x3P2A).v5 | | | | |
| | | | Th. Max. | Th. Max. |
| Protein | | Length | GC (%) | GC (abs) |
| PumaBH3 | MEEQWA | 143 | 72.03 | 309 |
| (x3P2A).v5 | | | | |

| | | | U | U | U Content | | UU | | | |
|---|---|---|---|---|---|---|---|---|---|---|
| Nucleic | | | Content | Content | v Th. Min | UU | pairs v | | | |
| Acid | | Length | (abs) | (%) | (%) | Pairs | WT (%) | UUU | UUUU | UUUUU |
| PumaBH3 | ATGGAGG | 429 | 59 | 13.75 | 151.28 | 6 | 40.00 | 1 | 0 | 0 |
| (x3P2A).v5 | | | | | | | | | | |

| | | | G | G | G Content |
|---|---|---|---|---|---|
| Nucleic | | | Content | Content | v Th. Max |
| Acid | | Length | (abs) | (%) | (%) |
| PumaBH3 | ATGGAGG | 429 | 144 | 33.57 | 68.15 |
| (x3P2A).v5 | | | | | |
| | | | C | C | C Content |
| Nucleic | | | Content | Content | v Th. Max |
| Acid | | Length | (abs) | (%) | (%) |
| PumaBH3 | ATGGAGG | 429 | 121 | 28.21 | 52.05 |
| (x3P2A).v5 | | | | | |
| | | | | | GC |
| | | | GC | GC | Content v |
| Nucleic | | | Content | Content | Th. Max |
| Acid | | Length | (abs) | (%) | (%) |
| PumaBH3 | ATGGAGG | 429 | 265 | 61.77 | 74.69 |
| (x3P2A).v5 | | | | | |

In some embodiments, the uracil or thymine content (% U_{TL} or % T_{TL}) of a uracil- or thymine-modified sequence encoding a multimer polypeptide of the invention (e.g., PUMA-BH3 multimer polypeptide) is between 10% and 20%, between 11% and 20%, between 11.5% and 19.5%, between 12% and 19%, between 12.5% and 18.5%, between 13% and 18%, between 13% and 17%, between 13% and 16.5%, between 13% and 16%, between 13% and 15.5%, between 13% and 15%, or between 13% and 14.5%.

In some embodiments, the uracil or thymine content (% U_{TL} or % T_{TL}) of a uracil- or thymine-modified sequence encoding a multimer polypeptide of the invention (e.g., PUMA-BH3 multimer polypeptide) is between 12% and 15.5%, between 12.1% and 15.4%, between 12.2% and 15.3%, between 12.3% and 15.2%, between 12.4% and 15.1%, between 12.5% and 15%, between 12.6% and 14.9%, between 12.7% and 14.8%, between 12.8% and 14.7%, between 12.9% and 14.6%, or between 13% and 14.5%.

In a particular embodiment, the uracil or thymine content (% U_{TL} or % T_{TL}) of a uracil- or thymine modified sequence encoding a multimer polypeptide of the invention (e.g., PUMA-BH3 multimer polypeptide is between about 13% and about 15%, e.g., between 13.02% and 14.5%.

A uracil- or thymine-modified sequence encoding a polypeptide of the invention (e.g., a BH3 polypeptide, e.g., PUMA-BH3 multimer) can also be described according to its uracil or thymine content relative to the uracil or thymine content in the corresponding wild-type nucleic acid sequence (% U_{WT} or % T_{WT}), or according to its uracil or thymine content relative to the theoretical minimum uracil or thymine content of a nucleic acid encoding the wild-type protein sequence (% U_{TM} or (% T_{TM}).

The phrases “uracil or thymine content relative to the uracil or thymine content in the wild type nucleic acid sequence,” refers to a parameter determined by dividing the number of uracils or thymines in a sequence-optimized nucleic acid by the total number of uracils or thymines in the corresponding wild-type nucleic acid sequence and multiplying by 100. This parameter is abbreviated herein as % U_{WT} or % T_{WT}.

In some embodiments, the % U_{WT} or % T_{WT} of a uracil- or thymine-modified sequence encoding a polypeptide of the invention (e.g., a BH3 polypeptide, e.g., PUMA-BH3 multimer) is above 50%, above 55%, above 60%, above 65%, above 70%, above 75%, above 80%, above 85%, above 90%, or above 95%.

In some embodiments, the % U_{WT} or % T_{WT} of a uracil- or thymine modified sequence encoding a polypeptide of the invention (e.g., a BH3 polypeptide, e.g., PUMA-BH3 multimer) is between 55% and 85%, between 56% and 84%, between 57% and 83%, between 58% and 82%, between 59% and 81%, between 60% and 80%, between 61% and 79%, between 62% and 78%, between 63% and 77%, between 64% and 76%, between 65% and 75%, or between 65% and 74%.

In some embodiments, the % U_{WT} or % T_{WT} of a uracil- or thymine-modified sequence encoding a polypeptide of the invention (e.g., a BH3 polypeptide, e.g., PUMA-BH3 multimer) is between 63% and 75%, between 63.2% and 74.8%, between 63.4% and 74.6%, between 63.6% and 74.4%, between 63.8% and 74.2%, between 64% and 74%, between 64.2% and 73.8%, between 64.4% and 73.6%, between 64.6% and 73.4%, between 64.8% and 73.2%, or between 65% and 73%.

In a particular embodiment, the % U_{WT} or % T_{WT} of a uracil- or thymine-modified sequence encoding a polypeptide of the invention (e.g., a BH3 polypeptide, e.g., PUMA-BH3 multimer) is between about 65% and about 73%, e.g., between 65.58% and 73.02%.

Uracil- or thymine-content relative to the uracil or thymine theoretical minimum, refers to a parameter determined by dividing the number of uracils or thymines in a sequence-optimized nucleotide sequence by the total number of uracils or thymines in a hypothetical nucleotide sequence in which all the codons in the hypothetical sequence are replaced with synonymous codons having the lowest possible uracil or thymine content and multiplying by 100. This parameter is abbreviated herein as % U_{TM} or % T_{TM}.

For DNA it is recognized that thymine is present instead of uracil, and one would substitute T where U appears. Thus, all the disclosures related to, e.g., % U_{TM}, % U_{WT}, or % U_{TL}, with respect to RNA are equally applicable to % T_{TM}, % T_{WT}, or % T_{TL} with respect to DNA.

In some embodiments, the % U_{TM} of a uracil-modified sequence encoding a polypeptide of the invention (e.g., a BH3 polypeptide, e.g., PUMA-BH3 multimer) is below 300%, below 295%, below 290%, below 285%, below 280%, below 275%, below 270%, below 265%, below 260%, below 255%, below 250%, below 245%, below 240%, below 235%, below 230%, below 225%, below 220%, below 215%, below 200%, below 195%, below 190%, below 185%, below 180%, below 175%, below 170%, below 165%, below 160%, below 155%, below 150%, below 145%, below 140%, below 139%, below 138%, below 137%, below 136%, below 135%, below 134%, below 133%, below 132%, below 131%, below 130%, below 129%, below 128%, below 127%, below 126%, below 125%, below 124%, below 123%, below 122%, below 121%, below 120%, below 119%, below 118%, below 117%, below 116%, or below 115%.

In some embodiments, the % U_{TM} of a uracil-modified sequence encoding a polypeptide of the invention (e.g., a BH3 polypeptide, e.g., PUMA-BH3 multimer) is above 100%, above 101%, above 102%, above 103%, above 104%, above 105%, above 106%, above 107%, above 108%, above 109%, above 110%, above 111%, above 112%, above 113%, above 114%, above 115%, above 116%, above 117%, above 118%, above 119%, above 120%, above 121%, above 122%, above 123%, above 124%, above 125%, or above 126%, above 127%, above 128%, above 129%, or above 130%, above 135%, above 130%, above 131%, above 132%, above 133%, above 134%, or above 135%.

In some embodiments, the % U_{TM} of a uracil-modified sequence encoding a polypeptide of the invention (e.g., a BH3 polypeptide, e.g., PUMA-BH3 multimer) is between 125% and 127%, between 124% and 128%, between 123% and 129%, between 122% and 130%, between 121% and 131%, between 120% and 132%, between 119% and 133%, between 118% and 134%, between 117% and 135%, between 116% and 136%, between 115% and 137%, between 114% and 138%, or between 113% and 139%.

In some embodiments, a uracil-modified sequence encoding aa polypeptide of the invention (e.g., a BH3 polypeptide, e.g., PUMA-BH3 multimer) has a reduced number of consecutive uracils with respect to the corresponding wild-type nucleic acid sequence. For example, two consecutive leucines can be encoded by the sequence CUUUUG, which includes a four uracil cluster. Such a subsequence can be substituted, e.g., with CUGCUC, which removes the uracil cluster.

Phenylalanine can be encoded by UUC or UUU. Thus, even if phenylalanines encoded by UUU are replaced by UUC, the synonymous codon still contains a uracil pair (UU). Accordingly, the number of phenylalanines in a sequence establishes a minimum number of uracil pairs (UU) that cannot be eliminated without altering the number of phenylalanines in the encoded polypeptide. For example, if the polypeptide (e.g., wild type PUMA-BH3 multimer) has, e.g., 7, 8, or 9 phenylalanines, the absolute minimum number of uracil pairs (UU) in that a uracil-modified sequence encoding the polypeptide (e.g., wild type PUMA-BH3 multimer) can contain is 7, 8, or 9, respectively.

Wild type PUMA-BH3 multimer contains 6 uracil pairs (UU), and one uracil triplet (UUU). In some embodiments, a uracil-modified sequence encoding a PUMA-BH3 multimer polypeptide of the invention has a reduced number of uracil triplets (UUU) with respect to the wild-type nucleic acid sequence. In some embodiments, a uracil-modified sequence encoding a PUMA-BH3 multimer polypeptide of the invention contains 1 or no uracil triplets (UUU).

In some embodiments, a uracil-modified sequence encoding a PUMA-BH3 multimer polypeptide has a reduced number of uracil pairs (UU) with respect to the number of uracil pairs (UU) in the wild-type nucleic acid sequence. In some embodiments, a uracil-modified sequence encoding a PUMA-BH3 multimer polypeptide of the invention has a number of uracil pairs (UU) corresponding to the minimum possible number of uracil pairs (UU) in the wild-type nucleic acid sequence, e.g., 4 uracil pairs in the case of wild type PUMA-BH3 multimer.

In some embodiments, a uracil-modified sequence encoding a PUMA-BH3 multimer polypeptide of the invention has at least 1, 2, 3, 4, or 5 uracil pairs (UU) less than the number of uracil pairs (UU) in the wild-type nucleic acid sequence. In some embodiments, a uracil-modified sequence encoding a PUMA-BH3 multimer polypeptide of the invention has between 3 and 5 uracil pairs (UU).

The phrase “uracil pairs (UU) relative to the uracil pairs (UU) in the wild type nucleic acid sequence,” refers to a parameter determined by dividing the number of uracil pairs (UU) in a sequence-optimized nucleotide sequence by the total number of uracil pairs (UU) in the corresponding wild-type nucleotide sequence and multiplying by 100. This parameter is abbreviated herein as % UU_{wt}.

In some embodiments, a uracil-modified sequence encoding a polypeptide of the invention (e.g., a BH3 polypeptide, e.g., PUMA-BH3 multimer) has a % UU_{wt} less than 40%, less than 30%, or less than 20%. In some embodiments, a uracil-modified sequence encoding a multimer polypeptide (e.g., PUMA-BH3 multimer) has a % UU_{wt} between 20% and 40% In a particular embodiment, a uracil-modified sequence encoding a multimer polypeptide of the invention (e.g., PUMA-BH3 multimer) has a % UU_{wt} between 25% and 35%.

In some embodiments, the polynucleotide of the invention comprises a uracil-modified sequence encoding an intracellular binding polypeptide (e.g., PUMA-BH3 multimer polypeptide) disclosed herein. In some embodiments, the uracil-modified sequence encoding an intracellular binding polypeptide comprises at least one chemically modified nucleobase, e.g., 5-methoxyuracil. In some embodiments, at least 95% of a nucleobase (e.g., uracil) in a uracil-modified sequence encoding an intracellular binding polypeptide of the disclosure are modified nucleobases. In some embodiments, at least 95% of uracil in a uracil-modified sequence encoding an intracellular binding polypeptide is 5-methoxyuracil. In some embodiments, the polynucleotide comprising a uracil-modified sequence further comprises a miRNA binding site, e.g., a miRNA binding site that binds to miR-142. In some embodiments, the polynucleotide comprising a uracil-modified sequence is formulated with a delivery agent, e.g., a compound having Formula (I), e.g., any of Compounds 1-147.

In some embodiments, the “guanine content of the sequence optimized ORF encoding intracellular binding polypeptide with respect to the theoretical maximum guanine content of a nucleotide sequence encoding the intracellular binding polypeptide,” abbreviated as % G_{TMX} is at least 71%, at least 75%, at least about 80%, at least about 85%, at least about 90%, at least about 95%, or about 100%. In some embodiments, the % G_{TMX} is between about 71% and about 79%, between about 71% and about 78%, or between about 71% and about 77%.

In some embodiments, the “cytosine content of the ORF relative to the theoretical maximum cytosine content of a nucleotide sequence encoding the intracellular binding polypeptide,” abbreviated as % C_{TMX}, is at least 65%, at least about 70%, at least about 75%, at least about 80%, at least about 85%, at least about 90%, at least about 95%, or about 100%. In some embodiments, the % C_{TMX} is between about 65% and about 80%, between about 66% and about 80%, between about 67% and about 79%, or between about 68% and about 76%.

In some embodiments, the “guanine and cytosine content (G/C) of the ORF relative to the theoretical maximum G/C content in a nucleotide sequence encoding the intracellular binding polypeptide,” abbreviated as % G/C_{TMX} is at least about 86%, at least about 90%, at least about 95%, or about 100%. The % G/C_{TMX} is between about 86% and about 100%, between about 87% and about 99%, between about 90% and about 97%, or between about 91% and about 96%.

In some embodiments, the “G/C content in the ORF relative to the G/C content in the corresponding wild-type ORF,” abbreviated as % G/C_{WT} is at least 61%, at least 62%, at least 63%, at least 64%, at least 65%, at least 66%, at least 67%, at least 70, or at least 75%.

In some embodiments, the average G/C content in the 3rd codon position in the ORF is at least 20%, at least 21%, at least 22%, at least 23%, at least 24%, at least 25%, at least 26%, at least 27%, at least 28%, at least 29%, or at least 30% higher than the average G/C content in the 3rd codon position in the corresponding wild-type ORF.

In some embodiments, the polynucleotide of the invention comprises an open reading frame (ORF) encoding an intracellular binding polypeptide (e.g., PUMA-BH3 multimer), wherein the ORF has been sequence optimized, and wherein each of % U_{TL}, % U_{WT}, % U_{TM}, % G_{TL}, % G_{WT}, % G_{TMX}, % C_{TL}, % C_{WT}, % C_{TMX}, % G/C_{TL}, % G/C_{WT}, or % G/C_{TMX}, alone or in a combination thereof is in a range between (i) a maximum corresponding to the parameter's maximum value (MAX) plus about 0.5, 1, 1.5, 2, 2.5, 3, 3.5, 4, 4.5, 5, 5.5, 6, 6.5, 7, 7.5, 8, 8.5, 9, 9.5, or 10 standard deviations (STD DEV), and (ii) a minimum corresponding to the parameter's minimum value (MIN) less 0.5, 1, 1.5, 2, 2.5, 3, 3.5, 4, 4.5, 5, 5.5, 6, 6.5, 7, 7.5, 8, 8.5, 9, 9.5, or 10 standard deviations (STD DEV).

### Methods for Sequence Optimization

In some embodiments, a polynucleotide of the invention (e.g., a polynucleotide comprising a nucleotide sequence encoding a BH3 polypeptide, e.g., the wild-type sequence, functional fragment, or variant thereof) is sequence optimized. A sequence optimized nucleotide sequence (nucleotide sequence is also referred to as “nucleic acid” herein) comprises at least one codon modification with respect to a reference sequence (e.g., a wild-type sequence encoding a BH3 polypeptide). Thus, in a sequence optimized nucleic acid, at least one codon is different from a corresponding codon in a reference sequence (e.g., a wild-type sequence).

In general, sequence optimized nucleic acids are generated by at least a step comprising substituting codons in a reference sequence with synonymous codons (i.e., codons that encode the same amino acid). Such substitutions can be effected, for example, by applying a codon substitution map (i.e., a table providing the codons that will encode each amino acid in the codon optimized sequence), or by applying a set of rules (e.g., if glycine is next to neutral amino acid, glycine would be encoded by a certain codon, but if it is next to a polar amino acid, it would be encoded by another codon). In addition to codon substitutions (i.e., “codon optimization”) the sequence optimization methods disclosed herein comprise additional optimization steps which are not strictly directed to codon optimization such as the removal of deleterious motifs (destabilizing motif substitution). Compositions and formulations comprising these sequence optimized nucleic acids (e.g., a RNA, e.g., a mRNA) can be administered to a subject in need thereof to facilitate in vivo expression of functionally active polypeptides (e.g., BH3).

The recombinant expression of large molecules in cell cultures can be a challenging task with numerous limitations (e.g., poor protein expression levels, stalled translation resulting in truncated expression products, protein misfolding, etc.). These limitations can be reduced or avoided by administering the polynucleotides (e.g., a RNA, e.g., a mRNA), which encode a functionally active polypeptide (e.g., BH3) or compositions or formulations comprising the same to a patient suffering from AIP, so the synthesis and delivery of the polypeptide (e.g., BH3) to treat AIP takes place endogenously.

Changing from an in vitro expression system (e.g., cell culture) to in vivo expression requires the redesign of the nucleic acid sequence encoding the therapeutic agent. Redesigning a naturally occurring gene sequence by choosing different codons without necessarily altering the encoded amino acid sequence can often lead to dramatic increases in protein expression levels (Gustafsson et al., 2004, Trends Biotechnol 22:346-53). Variables such as codon adaptation index (CAI), mRNA secondary structures, cis-regulatory sequences, GC content and many other similar variables have been shown to somewhat correlate with protein expression levels (Villalobos et al., 2006, BMC Bioinformatics 7:285). However, due to the degeneracy of the genetic code, there are numerous different nucleic acid sequences that can all encode the same therapeutic agent. Each amino acid is encoded by up to six synonymous codons; and the choice between these codons influences gene expression. In addition, codon usage (i.e., the frequency with which different organisms use codons for expressing a polypeptide sequence) differs among organisms (for example, recombinant production of human or humanized therapeutic antibodies frequently takes place in hamster cell cultures).

In some embodiments, a reference nucleic acid sequence can be sequence optimized by applying a codon map. The skilled artisan will appreciate that T bases are present in DNA, whereas the T bases would be replaced by U bases in corresponding RNAs. For example, a sequence optimized nucleic acid disclosed herein in DNA form, e.g., a vector or an in-vitro translation (IVT) template, would have its T bases transcribed as U based in its corresponding transcribed mRNA. In this respect, both sequence optimized DNA sequences (comprising T) and their corresponding RNA sequences (comprising U) are considered sequence optimized nucleic acid of the present invention. A skilled artisan would also understand that equivalent codon-maps can be generated by replaced one or more bases with non-natural bases. Thus, e.g., a TTC codon (DNA map) would correspond to a UUC codon (RNA map), which in turn can correspond to a ΨΨC codon (RNA map in which U has been replaced with pseudouridine).

In one embodiment, a reference sequence encoding BH3 can be optimized by replacing all the codons encoding a certain amino acid with only one of the alternative codons provided in a codon map. For example, all the valines in the optimized sequence would be encoded by GTG or GTC or GTT.

Sequence optimized polynucleotides of the invention can be generated using one or more optimization methods, or a combination thereof. Sequence optimization methods which can be used to sequence optimize nucleic acid sequences are described in detail herein. This list of methods is not comprehensive or limiting.

It will be appreciated that the design principles and rules described for each one of the sequence optimization methods discussed below can be combined in many different ways, for example high G/C content sequence optimization for some regions or uridine content sequence optimization for other regions of the reference nucleic acid sequence, as well as targeted nucleotide mutations to minimize secondary structure throughout the sequence or to eliminate deleterious motifs.

The choice of potential combinations of sequence optimization methods can be, for example, dependent on the specific chemistry used to produce a synthetic polynucleotide. Such a choice can also depend on characteristics of the protein encoded by the sequence optimized nucleic acid, e.g., a full sequence, a functional fragment, or a fusion protein comprising BH3, etc. In some embodiments, such a choice can depend on the specific tissue or cell targeted by the sequence optimized nucleic acid (e.g., a therapeutic synthetic mRNA).

The mechanisms of combining the sequence optimization methods or design rules derived from the application and analysis of the optimization methods can be either simple or complex. For example, the combination can be:
(i) Sequential: Each sequence optimization method or set of design rules applies to a different subsequence of the overall sequence, for example reducing uridine at codon positions 1 to 30 and then selecting high frequency codons for the remainder of the sequence;
(ii) Hierarchical: Several sequence optimization methods or sets of design rules are combined in a hierarchical, deterministic fashion. For example, use the most GC-rich codons, breaking ties (which are common) by choosing the most frequent of those codons.
(iii) Multifactorial/Multiparametric: Machine learning or other modeling techniques are used to design a single sequence that best satisfies multiple overlapping and possibly contradictory requirements. This approach would require the use of a computer applying a number of mathematical techniques, for example, genetic algorithms.

Ultimately, each one of these approaches can result in a specific set of rules which in many cases can be summarized in a single codon table, i.e., a sorted list of codons for each amino acid in the target protein (i.e., BH3), with a specific rule or set of rules indicating how to select a specific codon for each amino acid position.

a. Uridine Content Optimization

The presence of local high concentrations of uridine in a nucleic acid sequence can have detrimental effects on translation, e.g., slow or prematurely terminated translation, especially when modified uridine analogs are used in the production of synthetic mRNAs. Furthermore, high uridine content can also reduce the in vivo half-life of synthetic mRNAs due to TLR activation.

Accordingly, a nucleic acid sequence can be sequence optimized using a method comprising at least one uridine content optimization step. Such a step comprises, e.g., substituting at least one codon in the reference nucleic acid with an alternative codon to generate a uridine-modified sequence, wherein the uridine-modified sequence has at least one of the following properties:
(i) increase or decrease in global uridine content;
(ii) increase or decrease in local uridine content (i.e., changes in uridine content are limited to specific subsequences);
(iii) changes in uridine distribution without altering the global uridine content;
(iv) changes in uridine clustering (e.g., number of clusters, location of clusters, or distance between clusters); or
(v) combinations thereof.

In some embodiments, the sequence optimization process comprises optimizing the global uridine content, i.e., optimizing the percentage of uridine nucleobases in the sequence optimized nucleic acid with respect to the percentage of uridine nucleobases in the reference nucleic acid sequence. For example, 30% of nucleobases can be uridines in the reference sequence and 10% of nucleobases can be uridines in the sequence optimized nucleic acid.

In other embodiments, the sequence optimization process comprises reducing the local uridine content in specific regions of a reference nucleic acid sequence, i.e., reducing the percentage of uridine nucleobases in a subsequence of the sequence optimized nucleic acid with respect to the percentage of uridine nucleobases in the corresponding subsequence of the reference nucleic acid sequence. For example, the reference nucleic acid sequence can have a 5′-end region (e.g., 30 codons) with a local uridine content of 30%, and the uridine content in that same region could be reduced to 10% in the sequence optimized nucleic acid.

In specific embodiments, codons can be replaced in the reference nucleic acid sequence to reduce or modify, for example, the number, size, location, or distribution of uridine clusters that could have deleterious effects on protein translation. Although as a general rule it is desirable to reduce the uridine content of the reference nucleic acid sequence, in certain embodiments the uridine content, and in particular the local uridine content, of some subsequences of the reference nucleic acid sequence can be increased.

The reduction of uridine content to avoid adverse effects on translation can be done in combination with other optimization methods disclosed here to achieve other design goals. For example, uridine content optimization can be combined with ramp design, since using the rarest codons for most amino acids will, with a few exceptions, reduce the U content.

In some embodiments, the uridine-modified sequence is designed to induce a lower Toll-Like Receptor (TLR) response when compared to the reference nucleic acid sequence. Several TLRs recognize and respond to nucleic acids. Double-stranded (ds)RNA, a frequent viral constituent, has been shown to activate TLR3. See Alexopoulou et al. (2001) Nature, 413:732-738 and Wang et al. (2004) Nat. Med., 10:1366-1373. Single-stranded (ss)RNA activates TLR7. See Diebold et al. (2004) Science 303:1529-1531. RNA oligonucleotides, for example RNA with phosphorothioate internucleotide linkages, are ligands of human TLR8. See Heil et al. (2004) Science 303:1526-1529. DNA containing unmethylated CpG motifs, characteristic of bacterial and viral DNA, activate TLR9. See Hemmi et al. (2000) Nature, 408: 740-745.

As used herein, the term “TLR response” is defined as the recognition of single-stranded RNA by a TLR7 receptor, and in some embodiments encompasses the degradation of the RNA and/or physiological responses caused by the recognition of the single-stranded RNA by the receptor. Methods to determine and quantitate the binding of an RNA to a TLR7 are known in the art. Similarly, methods to determine whether an RNA has triggered a TLR7-mediated physiological response (e.g., cytokine secretion) are well known in the art. In some embodiments, a TLR response can be mediated by TLR3, TLR8, or TLR9 instead of TLR7.

Suppression of TLR7-mediated response can be accomplished via nucleoside modification. RNA undergoes over hundred different nucleoside modifications in nature (see the RNA Modification Database, available at mods.rna.albany.edu). Human rRNA, for example, has ten times more pseudouridine (Ψ) and 25 times more 2′-O-methylated nucleosides than bacterial rRNA. Bacterial mRNA contains no nucleoside modifications, whereas mammalian mRNAs have modified nucleosides such as 5-methylcytidine (m5C), N6-methyladenosine (m6A), inosine and many 2′-O-methylated nucleosides in addition to N7-methylguanosine (m7G).

Uracil and ribose, the two defining features of RNA, are both necessary and sufficient for TLR7 stimulation, and short single-stranded RNA (ssRNA) act as TLR7 agonists in a sequence-independent manner as long as they contain several uridines in close proximity. See Diebold et al. (2006) Eur. J. Immunol. 36:3256-3267, which is herein incorporated by reference in its entirety. Accordingly, one or more of the optimization methods disclosed herein comprises reducing the uridine content (locally and/or globally) and/or reducing or modifying uridine clustering to reduce or to suppress a TLR7-mediated response.

In some embodiments, the TLR response (e.g., a response mediated by TLR7) caused by the uridine-modified sequence is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% lower than the TLR response caused by the reference nucleic acid sequence.

In some embodiments, the TLR response caused by the reference nucleic acid sequence is at least about 1-fold, at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 3-fold, at least about 4-fold, at least about 5-fold, at least about 6-fold, at least about 7-fold, at least about 8-fold, at least about 9-fold, or at least about 10-fold higher than the TLR response caused by the uridine-modified sequence.

In some embodiments, the uridine content (average global uridine content) (absolute or relative) of the uridine-modified sequence is higher than the uridine content (absolute or relative) of the reference nucleic acid sequence. Accordingly, in some embodiments, the uridine-modified sequence contains at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% more uridine that the reference nucleic acid sequence.

In other embodiments, the uridine content (average global uridine content) (absolute or relative) of the uridine-modified sequence is lower than the uridine content (absolute or relative) of the reference nucleic acid sequence. Accordingly, in some embodiments, the uridine-modified sequence contains at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% less uridine that the reference nucleic acid sequence.

In some embodiments, the uridine content (average global uridine content) (absolute or relative) of the uridine-modified sequence is less than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% of the total nucleobases in the uridine-modified sequence. In some embodiments, the uridine content of the uridine-modified sequence is between about 10% and about 20%. In some particular embodiments, the uridine content of the uridine-modified sequence is between about 12% and about 16%.

In some embodiments, the uridine content of the reference nucleic acid sequence can be measured using a sliding window. In some embodiments, the length of the sliding window is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleobases. In some embodiments, the sliding window is over 40 nucleobases in length. In some embodiments, the sliding window is 20 nucleobases in length. Based on the uridine content measured with a sliding window, it is possible to generate a histogram representing the uridine content throughout the length of the reference nucleic acid sequence and sequence optimized nucleic acids.

In some embodiments, a reference nucleic acid sequence can be modified to reduce or eliminate peaks in the histogram that are above or below a certain percentage value. In some embodiments, the reference nucleic acid sequence can be modified to eliminate peaks in the sliding-window representation which are above 65%, 60%, 55%, 50%, 45%, 40%, 35%, or 30% uridine. In another embodiment, the reference nucleic acid sequence can be modified so no peaks are over 30% uridine in the sequence optimized nucleic acid, as measured using a 20 nucleobase sliding window. In some embodiments, the reference nucleic acid sequence can be modified so no more or no less than a predetermined number of peaks in the sequence optimized nucleic sequence, as measured using a 20 nucleobase sliding window, are above or below a certain threshold value. For example, in some embodiments, the reference nucleic acid sequence can be modified so no peaks or no more than 1, 2, 3, 4, 5, 6, 7, 8, 9 or 10 peaks in the sequence optimized nucleic acid are above 10%, 15%, 20%, 25% or 30% uridine. In another embodiment, the sequence optimized nucleic acid contains between 0 peaks and 2 peaks with uridine contents 30% of higher.

In some embodiments, a reference nucleic acid sequence can be sequence optimized to reduce the incidence of consecutive uridines. For example, two consecutive leucines could be encoded by the sequence CUUUUG, which would include a four uridine cluster. Such subsequence could be substituted with CUGCUC, which would effectively remove the uridine cluster. Accordingly, a reference nucleic sequence can be sequence optimized by reducing or eliminating uridine pairs (UU), uridine triplets (UUU) or uridine quadruplets (UUUU). Higher order combinations of U are not considered combinations of lower order combinations. Thus, for example, UUUU is strictly considered a quadruplet, not two consecutive U pairs; or UUUUUU is considered a sextuplet, not three consecutive U pairs, or two consecutive U triplets, etc.

In some embodiments, all uridine pairs (UU) and/or uridine triplets (UUU) and/or uridine quadruplets (UUUU) can be removed from the reference nucleic acid sequence. In other embodiments, uridine pairs (UU) and/or uridine triplets (UUU) and/or uridine quadruplets (UUUU) can be reduced below a certain threshold, e.g., no more than 1, 2, 3, 4, 5, 6, 7, 8, 9, 10, 11, 12, 13, 14, 15, 16, 17, 18, 19, or 20 occurrences in the sequence optimized nucleic acid. In a particular embodiment, the sequence optimized nucleic acid contains less than 20, 19, 18, 17, 16, 15, 14, 13, 12, 11, 10, 9, 8, 7, 6, 5, 4, 3, 2, or 1 uridine pairs. In another particular embodiment, the sequence optimized nucleic acid contains no uridine pairs and/or triplets.

Phenylalanine codons, i.e., UUC or UUU, comprise a uridine pair or triplet and therefore sequence optimization to reduce uridine content can at most reduce the phenylalanine U triplet to a phenylalanine U pair. In some embodiments, the occurrence of uridine pairs (UU) and/or uridine triplets (UUU) refers only to non-phenylalanine U pairs or triplets. Accordingly, in some embodiments, non-phenylalanine uridine pairs (UU) and/or uridine triplets (UUU) can be reduced below a certain threshold, e.g., no more than 1, 2, 3, 4, 5, 6, 7, 8, 9, 10, 11, 12, 13, 14, 15, 16, 17, 18, 19, or 20 occurrences in the sequence optimized nucleic acid. In a particular embodiment, the sequence optimized nucleic acid contains less than 20, 19, 18, 17, 16, 15, 14, 13, 12, 11, 10, 9, 8, 7, 6, 5, 4, 3, 2, or 1 non-phenylalanine uridine pairs and/or triplets. In another particular embodiment, the sequence optimized nucleic acid contains no non-phenylalanine uridine pairs and/or triplets.

In some embodiments, the reduction in uridine combinations (e.g., pairs, triplets, quadruplets) in the sequence optimized nucleic acid can be expressed as a percentage reduction with respect to the uridine combinations present in the reference nucleic acid sequence.

In some embodiments, a sequence optimized nucleic acid can contain about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, or 65% of the total number of uridine pairs present in the reference nucleic acid sequence. In some embodiments, a sequence optimized nucleic acid can contain about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, or 65% of the total number of uridine triplets present in the reference nucleic acid sequence. In some embodiments, a sequence optimized nucleic acid can contain about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, or 65% of the total number of uridine quadruplets present in the reference nucleic acid sequence.

In some embodiments, a sequence optimized nucleic acid can contain about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, or 65% of the total number of non-phenylalanine uridine pairs present in the reference nucleic acid sequence. In some embodiments, a sequence optimized nucleic acid can contain about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, or 65% of the total number of non-phenylalanine uridine triplets present in the reference nucleic acid sequence.

In some embodiments, the uridine content in the sequence optimized sequence can be expressed with respect to the theoretical minimum uridine content in the sequence. The term “theoretical minimum uridine content” is defined as the uridine content of a nucleic acid sequence as a percentage of the sequence's length after all the codons in the sequence have been replaced with synonymous codon with the lowest uridine content. In some embodiments, the uridine content of the sequence optimized nucleic acid is identical to the theoretical minimum uridine content of the reference sequence (e.g., a wild type sequence). In some aspects, the uridine content of the sequence optimized nucleic acid is about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200%, about 210%, about 220%, about 230%, about 240% or about 250% of the theoretical minimum uridine content of the reference sequence (e.g., a wild type sequence).

In some embodiments, the uridine content of the sequence optimized nucleic acid is identical to the theoretical minimum uridine content of the reference sequence (e.g., a wild type sequence). The reference nucleic acid sequence (e.g., a wild type sequence) can comprise uridine clusters which due to their number, size, location, distribution or combinations thereof have negative effects on translation. As used herein, the term “uridine cluster” refers to a subsequence in a reference nucleic acid sequence or sequence optimized nucleic sequence with contains a uridine content (usually described as a percentage) which is above a certain threshold. Thus, in certain embodiments, if a subsequence comprises more than about 10%, 15%, 20%, 25%, 30%, 35%, 40%, 45%, 50%, 55%, 60% or 65% uridine content, such subsequence would be considered a uridine cluster.

The negative effects of uridine clusters can be, for example, eliciting a TLR7 response. Thus, in some implementations of the nucleic acid sequence optimization methods disclosed herein it is desirable to reduce the number of clusters, size of clusters, location of clusters (e.g., close to the 5′ and/or 3′ end of a nucleic acid sequence), distance between clusters, or distribution of uridine clusters (e.g., a certain pattern of cluster along a nucleic acid sequence, distribution of clusters with respect to secondary structure elements in the expressed product, or distribution of clusters with respect to the secondary structure of an mRNA).

In some embodiments, the reference nucleic acid sequence comprises at least one uridine cluster, wherein said uridine cluster is a subsequence of the reference nucleic acid sequence wherein the percentage of total uridine nucleobases in said subsequence is above a predetermined threshold. In some embodiments, the length of the subsequence is at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 nucleobases. In some embodiments, the subsequence is longer than 100 nucleobases. In some embodiments, the threshold is 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% or 25% uridine content. In some embodiments, the threshold is above 25%.

For example, an amino acid sequence comprising A, D, G, S and R could be encoded by the nucleic acid sequence GCU, GAU, GGU, AGU, CGU. Although such sequence does not contain any uridine pairs, triplets, or quadruplets, one third of the nucleobases would be uridines. Such a uridine cluster could be removed by using alternative codons, for example, by using GCC, GAC, GGC, AGC, and CGC, which would contain no uridines.

In other embodiments, the reference nucleic acid sequence comprises at least one uridine cluster, wherein said uridine cluster is a subsequence of the reference nucleic acid sequence wherein the percentage of uridine nucleobases of said subsequence as measured using a sliding window that is above a predetermined threshold. In some embodiments, the length of the sliding window is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleobases. In some embodiments, the sliding window is over 40 nucleobases in length. In some embodiments, the threshold is 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% or 25% uridine content. In some embodiments, the threshold is above 25%.

In some embodiments, the reference nucleic acid sequence comprises at least two uridine clusters. In some embodiments, the uridine-modified sequence contains fewer uridine-rich clusters than the reference nucleic acid sequence. In some embodiments, the uridine-modified sequence contains more uridine-rich clusters than the reference nucleic acid sequence. In some embodiments, the uridine-modified sequence contains uridine-rich clusters with are shorter in length than corresponding uridine-rich clusters in the reference nucleic acid sequence. In other embodiments, the uridine-modified sequence contains uridine-rich clusters which are longer in length than the corresponding uridine-rich cluster in the reference nucleic acid sequence. See, Kariko et al. (2005) Immunity 23:165-175; Kormann et al. (2010) Nature Biotechnology 29:154-157; or Sahin et al. (2014) Nature Reviews Drug Discovery I AOP, published online 19 Sep. 2014m doi:10.1038/nrd4278; all of which are herein incorporated by reference their entireties.

b. Guanine/Cytosine (G/C) Content

A reference nucleic acid sequence can be sequence optimized using methods comprising altering the Guanine/Cytosine (G/C) content (absolute or relative) of the reference nucleic acid sequence. Such optimization can comprise altering (e.g., increasing or decreasing) the global G/C content (absolute or relative) of the reference nucleic acid sequence; introducing local changes in G/C content in the reference nucleic acid sequence (e.g., increase or decrease G/C in selected regions or subsequences in the reference nucleic acid sequence); altering the frequency, size, and distribution of G/C clusters in the reference nucleic acid sequence, or combinations thereof.

In some embodiments, the sequence optimized nucleic acid encoding a polypeptide (e.g., BH3) comprises an overall increase in G/C content (absolute or relative) relative to the G/C content (absolute or relative) of the reference nucleic acid sequence. In some embodiments, the overall increase in G/C content (absolute or relative) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% relative to the G/C content (absolute or relative) of the reference nucleic acid sequence.

In some embodiments, the sequence optimized nucleic acid encoding a polypeptide (e.g., BH3) comprises an overall decrease in G/C content (absolute or relative) relative to the G/C content of the reference nucleic acid sequence. In some embodiments, the overall decrease in G/C content (absolute or relative) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% relative to the G/C content (absolute or relative) of the reference nucleic acid sequence.

In some embodiments, the sequence optimized nucleic acid encoding a polypeptide (e.g., BH3) comprises a local increase in Guanine/Cytosine (G/C) content (absolute or relative) in a subsequence (i.e., a G/C modified subsequence) relative to the G/C content (absolute or relative) of the corresponding subsequence in the reference nucleic acid sequence. In some embodiments, the local increase in G/C content (absolute or relative) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% relative to the G/C content (absolute or relative) of the corresponding subsequence in the reference nucleic acid sequence.

In some embodiments, the sequence optimized nucleic acid encoding a polypeptide (e.g., BH3) comprises a local decrease in Guanine/Cytosine (G/C) content (absolute or relative) in a subsequence (i.e., a G/C modified subsequence) relative to the G/C content (absolute or relative) of the corresponding subsequence in the reference nucleic acid sequence. In some embodiments, the local decrease in G/C content (absolute or relative) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% relative to the G/C content (absolute or relative) of the corresponding subsequence in the reference nucleic acid sequence.

In some embodiments, the G/C content (absolute or relative) is increased or decreased in a subsequence which is at least about 5, 10, 15, 20, 25, 30, 35, 40, 45, 50, 55, 60, 65, 70, 75, 80, 85, 90, 95, or 100 nucleobases in length.

In some embodiments, the G/C content (absolute or relative) is increased or decreased in a subsequence which is at least about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleobases in length.

In some embodiments, the G/C content (absolute or relative) is increased or decreased in a subsequence which is at least about 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 nucleobases in length.

The increases or decreases in G and C content (absolute or relative) described herein can be conducted by replacing synonymous codons with low G/C content with synonymous codons having higher G/C content, or vice versa. For example, L has 6 synonymous codons: two of them have 2 G/C (CUC, CUG), 3 have a single G/C (UUG, CUU, CUA), and one has no G/C (UUA). So if the reference nucleic acid had a CUC codon in a certain position, G/C content at that position could be reduced by replacing CUC with any of the codons having a single G/C or the codon with no G/C. See, U.S. Publ. Nos. US20140228558, US20050032730 A1; Gustafsson et al. (2012) Protein Expression and Purification 83: 37-46; all of which are incorporated herein by reference in their entireties.

c. Codon Frequency—Codon Usage Bias

Numerous codon optimization methods known in the art are based on the substitution of codons in a reference nucleic acid sequence with codons having higher frequencies. Thus, in some embodiments, a nucleic acid sequence encoding a polypeptide (e.g., BH3) disclosed herein can be sequence optimized using methods comprising the use of modifications in the frequency of use of one or more codons relative to other synonymous codons in the sequence optimized nucleic acid with respect to the frequency of use in the non-codon optimized sequence.

As used herein, the term “codon frequency” refers to codon usage bias, i.e., the differences in the frequency of occurrence of synonymous codons in coding DNA/RNA. It is generally acknowledged that codon preferences reflect a balance between mutational biases and natural selection for translational optimization. Optimal codons help to achieve faster translation rates and high accuracy. As a result of these factors, translational selection is expected to be stronger in highly expressed genes. In the field of bioinformatics and computational biology, many statistical methods have been proposed and used to analyze codon usage bias. See, e.g., Comeron & Aguadé (1998) J. Mol. Evol. 47: 268-74. Methods such as the “frequency of optimal codons” (Fop) (Ikemura (1981) J. Mol. Biol. 151 (3): 389-409), the “Relative Codon Adaptation” (RCA) (Fox & Eril (2010) DNA Res. 17 (3): 185-96) or the “Codon Adaptation Index” (CAI) (Sharp & Li (1987) Nucleic Acids Res. 15 (3): 1281-95) are used to predict gene expression levels, while methods such as the “effective number of codons” (Nc) and Shannon entropy from information theory are used to measure codon usage evenness. Multivariate statistical methods, such as correspondence analysis and principal component analysis, are widely used to analyze variations in codon usage among genes (Suzuki et al. (2008) DNA Res. 15 (6): 357-65; Sandhu et al., In Silico Biol. 2008; 8(2):187-92).

The nucleic acid sequence encoding a polypeptide disclosed herein (e.g., a wild type nucleic acid sequence, a mutant nucleic acid sequence, a chimeric nucleic sequence, etc. which can be, for example, an mRNA), can be codon optimized using methods comprising substituting at least one codon in the reference nucleic acid sequence with an alternative codon having a higher or lower codon frequency in the synonymous codon set; wherein the resulting sequence optimized nucleic acid has at least one optimized property with respect to the reference nucleic acid sequence.

In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the codons in the reference nucleic acid sequence encoding a polypeptide (e.g., BH3) are substituted with alternative codons, each alternative codon having a codon frequency higher than the codon frequency of the substituted codon in the synonymous codon set.

In some embodiments, at least one codon in the reference nucleic acid sequence encoding a polypeptide (e.g., BH3) is substituted with an alternative codon having a codon frequency higher than the codon frequency of the substituted codon in the synonymous codon set, and at least one codon in the reference nucleic acid sequence is substituted with an alternative codon having a codon frequency lower than the codon frequency of the substituted codon in the synonymous codon set.

In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the codons in the reference nucleic acid sequence encoding a polypeptide (e.g., BH3) are substituted with alternative codons, each alternative codon having a codon frequency higher than the codon frequency of the substituted codon in the synonymous codon set.

In some embodiments, at least one alternative codon having a higher codon frequency has the highest codon frequency in the synonymous codon set. In other embodiments, all alternative codons having a higher codon frequency have the highest codon frequency in the synonymous codon set.

In some embodiments, at least one alternative codon having a lower codon frequency has the lowest codon frequency in the synonymous codon set. In some embodiments, all alternative codons having a higher codon frequency have the highest codon frequency in the synonymous codon set.

In some specific embodiments, at least one alternative codon has the second highest, the third highest, the fourth highest, the fifth highest or the sixth highest frequency in the synonymous codon set. In some specific embodiments, at least one alternative codon has the second lowest, the third lowest, the fourth lowest, the fifth lowest, or the sixth lowest frequency in the synonymous codon set.

Optimization based on codon frequency can be applied globally, as described above, or locally to the reference nucleic acid sequence encoding a polypeptide (e.g., BH3). In some embodiments, when applied locally, regions of the reference nucleic acid sequence can modified based on codon frequency, substituting all or a certain percentage of codons in a certain subsequence with codons that have higher or lower frequencies in their respective synonymous codon sets. Thus, in some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the codons in a subsequence of the reference nucleic acid sequence are substituted with alternative codons, each alternative codon having a codon frequency higher than the codon frequency of the substituted codon in the synonymous codon set.

In some embodiments, at least one codon in a subsequence of the reference nucleic acid sequence encoding a polypeptide (e.g., BH3) is substituted with an alternative codon having a codon frequency higher than the codon frequency of the substituted codon in the synonymous codon set, and at least one codon in a subsequence of the reference nucleic acid sequence is substituted with an alternative codon having a codon frequency lower than the codon frequency of the substituted codon in the synonymous codon set.

In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the codons in a subsequence of the reference nucleic acid sequence encoding a polypeptide (e.g., BH3) are substituted with alternative codons, each alternative codon having a codon frequency higher than the codon frequency of the substituted codon in the synonymous codon set.

In some embodiments, at least one alternative codon substituted in a subsequence of the reference nucleic acid sequence encoding a polypeptide (e.g., BH3) and having a higher codon frequency has the highest codon frequency in the synonymous codon set. In other embodiments, all alternative codons substituted in a subsequence of the reference nucleic acid sequence and having a lower codon frequency have the lowest codon frequency in the synonymous codon set.

In some embodiments, at least one alternative codon substituted in a subsequence of the reference nucleic acid sequence encoding a polypeptide (e.g., BH3) and having a lower codon frequency has the lowest codon frequency in the synonymous codon set. In some embodiments, all alternative codons substituted in a subsequence of the reference nucleic acid sequence and having a higher codon frequency have the highest codon frequency in the synonymous codon set.

In specific embodiments, a sequence optimized nucleic acid encoding a polypeptide (e.g., BH3) can comprise a subsequence having an overall codon frequency higher or lower than the overall codon frequency in the corresponding subsequence of the reference nucleic acid sequence at a specific location, for example, at the 5′ end or 3′ end of the sequence optimized nucleic acid, or within a predetermined distance from those region (e.g., at least 1, 2, 3, 4, 5, 6, 7, 8, 9, 10, 15, 20, 25, 30, 35, 40, 45, 50, 55, 60, 65, 70, 75, 80, 85, 90, 95 or 100 codons from the 5′ end or 3′ end of the sequence optimized nucleic acid).

In some embodiments, a sequence optimized nucleic acid encoding a polypeptide (e.g., BH3) can comprise more than one subsequence having an overall codon frequency higher or lower than the overall codon frequency in the corresponding subsequence of the reference nucleic acid sequence. A skilled artisan would understand that subsequences with overall higher or lower overall codon frequencies can be organized in innumerable patterns, depending on whether the overall codon frequency is higher or lower, the length of the subsequence, the distance between subsequences, the location of the subsequences, etc. See, U.S. Pat. Nos. 5,082,767, 8,126,653, 7,561,973, 8,401,798; U.S. Publ. No. US 20080046192, US 20080076161; Int'l. Publ. No. WO2000018778; Welch et al. (2009) PLoS ONE 4(9): e7002; Gustafsson et al. (2012) Protein Expression and Purification 83: 37-46; Chung et al. (2012) BMC Systems Biology 6:134; all of which are incorporated herein by reference in their entireties.

d. Destabilizing Motif Substitution

There is a variety of motifs that can affect sequence optimization, which fall into various non-exclusive categories, for example:
(i) Primary sequence based motifs: Motifs defined by a simple arrangement of nucleotides.
(ii) Structural motifs: Motifs encoded by an arrangement of nucleotides that tends to form a certain secondary structure.
(iii) Local motifs: Motifs encoded in one contiguous subsequence.
(iv) Distributed motifs: Motifs encoded in two or more disjoint subsequences.
(v) Advantageous motifs: Motifs which improve nucleotide structure or function.
(vi) Disadvantageous motifs: Motifs with detrimental effects on nucleotide structure or function.

There are many motifs that fit into the category of disadvantageous motifs. Some examples include, for example, restriction enzyme motifs, which tend to be relatively short, exact sequences such as the restriction site motifs for Xba1 (TCTAGA), EcoRI (GAATTC), EcoRII (CCWGG, wherein W means A or T, per the IUPAC ambiguity codes), or HindIII(AAGCTT); enzyme sites, which tend to be longer and based on consensus not exact sequence, such in the T7 RNA polymerase (GnnnnWnCRnCTCnCnnWnD, wherein n means any nucleotide, R means A or G, W means A or T, D means A or G or T but not C); structural motifs, such as GGGG repeats (Kim et al. (1991) Nature 351(6324):331-2); or other motifs such as CUG-triplet repeats (Querido et al. (2014) J. Cell Sci. 124:1703-1714).

Accordingly, the nucleic acid sequence encoding a polypeptide (e.g., BH3) disclosed herein can be sequence optimized using methods comprising substituting at least one destabilizing motif in a reference nucleic acid sequence, and removing such disadvantageous motif or replacing it with an advantageous motif.

In some embodiments, the optimization process comprises identifying advantageous and/or disadvantageous motifs in the reference nucleic sequence, wherein such motifs are, e.g., specific subsequences that can cause a loss of stability in the reference nucleic acid sequence prior or during the optimization process. For example, substitution of specific bases during optimization can generate a subsequence (motif) recognized by a restriction enzyme. Accordingly, during the optimization process the appearance of disadvantageous motifs can be monitored by comparing the sequence optimized sequence with a library of motifs known to be disadvantageous. Then, the identification of disadvantageous motifs could be used as a post-hoc filter, i.e., to determine whether a certain modification which potentially could be introduced in the reference nucleic acid sequence should be actually implemented or not.

In some embodiments, the identification of disadvantageous motifs can be used prior to the application of the sequence optimization methods disclosed herein, i.e., the identification of motifs in the reference nucleic acid sequence encoding a polypeptide (e.g., BH3) and their replacement with alternative nucleic acid sequences can be used as a preprocessing step, for example, before uridine reduction.

In other embodiments, the identification of disadvantageous motifs and their removal is used as an additional sequence optimization technique integrated in a multiparametric nucleic acid optimization method comprising two or more of the sequence optimization methods disclosed herein. When used in this fashion, a disadvantageous motif identified during the optimization process would be removed, for example, by substituting the lowest possible number of nucleobases in order to preserve as closely as possible the original design principle(s) (e.g., low U, high frequency, etc.). See, e.g., U.S. Publ. Nos. US20140228558, US20050032730, or US20140228558, which are herein incorporated by reference in their entireties.

e. Limited Codon Set Optimization

In some particular embodiments, sequence optimization of a reference nucleic acid sequence encoding a polypeptide (e.g., BH3) can be conducted using a limited codon set, e.g., a codon set wherein less than the native number of codons is used to encode the 20 natural amino acids, a subset of the 20 natural amino acids, or an expanded set of amino acids including, for example, non-natural amino acids.

The genetic code is highly similar among all organisms and can be expressed in a simple table with 64 entries which would encode the 20 standard amino acids involved in protein translation plus start and stop codons. The genetic code is degenerate, i.e., in general, more than one codon specifies each amino acid. For example, the amino acid leucine is specified by the UUA, UUG, CUU, CUC, CUA, or CUG codons, while the amino acid serine is specified by UCA, UCG, UCC, UCU, AGU, or AGC codons (difference in the first, second, or third position). Native genetic codes comprise 62 codons encoding naturally occurring amino acids. Thus, in some embodiments of the methods disclosed herein optimized codon sets (genetic codes) comprising less than 62 codons to encode 20 amino acids can comprise 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 30, 29, 28, 27, 26, 25, 24, 23, 22, 21, or 20 codons.

In some embodiments, the limited codon set comprises less than 20 codons. For example, if a protein contains less than 20 types of amino acids, such protein could be encoded by a codon set with less than 20 codons. Accordingly, in some embodiments, an optimized codon set comprises as many codons as different types of amino acids are present in the protein encoded by the reference nucleic acid sequence. In some embodiments, the optimized codon set comprises 19, 18, 17, 16, 15, 14, 13, 12, 11, 10, 9, 8, 7, 6, 5, 4, 3, 2 or even 1 codon.

In some embodiments, at least one amino acid selected from the group consisting of Ala, Arg, Asn, Asp, Cys, Gln, Glu, Gly, His, Ile, Leu, Lys, Phe, Pro, Ser, Thr, Tyr, and Val, i.e., amino acids which are naturally encoded by more than one codon, is encoded with less codons than the naturally occurring number of synonymous codons. For example, in some embodiments, Ala can be encoded in the sequence optimized nucleic acid by 3, 2 or 1 codons; Cys can be encoded in the sequence optimized nucleic acid by 1 codon; Asp can be encoded in the sequence optimized nucleic acid by 1 codon; Glu can be encoded in the sequence optimized nucleic acid by 1 codon; Phe can be encoded in the sequence optimized nucleic acid by 1 codon; Gly can be encoded in the sequence optimized nucleic acid by 3 codons, 2 codons or 1 codon; His can be encoded in the sequence optimized nucleic acid by 1 codon; Ile can be encoded in the sequence optimized nucleic acid by 2 codons or 1 codon; Lys can be encoded in the sequence optimized nucleic acid by 1 codon; Leu can be encoded in the sequence optimized nucleic acid by 5 codons, 4 codons, 3 codons, 2 codons or 1 codon; Asn can be encoded in the sequence optimized nucleic acid by 1 codon; Pro can be encoded in the sequence optimized nucleic acid by 3 codons, 2 codons, or 1 codon; Gln can be encoded in the sequence optimized nucleic acid by 1 codon; Arg can be encoded in the sequence optimized nucleic acid by 5 codons, 4 codons, 3 codons, 2 codons, or 1 codon; Ser can be encoded in the sequence optimized nucleic acid by 5 codons, 4 codons, 3 codons, 2 codons, or 1 codon; Thr can be encoded in the sequence optimized nucleic acid by 3 codons, 2 codons, or 1 codon; Val can be encoded in the sequence optimized nucleic acid by 3 codons, 2 codons, or 1 codon; and, Tyr can be encoded in the sequence optimized nucleic acid by 1 codon.

In some embodiments, at least one amino acid selected from the group consisting of Ala, Arg, Asn, Asp, Cys, Gln, Glu, Gly, His, Ile, Leu, Lys, Phe, Pro, Ser, Thr, Tyr, and Val, i.e., amino acids which are naturally encoded by more than one codon, is encoded by a single codon in the limited codon set.

In some specific embodiments, the sequence optimized nucleic acid is a DNA and the limited codon set consists of 20 codons, wherein each codon encodes one of 20 amino acids. In some embodiments, the sequence optimized nucleic acid is a DNA and the limited codon set comprises at least one codon selected from the group consisting of GCT, GCC, GCA, and GCG; at least a codon selected from the group consisting of CGT, CGC, CGA, CGG, AGA, and AGG; at least a codon selected from AAT or ACC; at least a codon selected from GAT or GAC; at least a codon selected from TGT or TGC; at least a codon selected from CAA or CAG; at least a codon selected from GAA or GAG; at least a codon selected from the group consisting of GGT, GGC, GGA, and GGG; at least a codon selected from CAT or CAC; at least a codon selected from the group consisting of ATT, ATC, and ATA; at least a codon selected from the group consisting of TTA, TTG, CTT, CTC, CTA, and CTG; at least a codon selected from AAA or AAG; an ATG codon; at least a codon selected from TTT or TTC; at least a codon selected from the group consisting of CCT, CCC, CCA, and CCG; at least a codon selected from the group consisting of TCT, TCC, TCA, TCG, AGT, and AGC; at least a codon selected from the group consisting of ACT, ACC, ACA, and ACG; a TGG codon; at least a codon selected from TAT or TAC; and, at least a codon selected from the group consisting of GTT, GTC, GTA, and GTG.

In other embodiments, the sequence optimized nucleic acid is an RNA (e.g., an mRNA) and the limited codon set consists of 20 codons, wherein each codon encodes one of 20 amino acids. In some embodiments, the sequence optimized nucleic acid is an RNA and the limited codon set comprises at least one codon selected from the group consisting of GCU, GCC, GCA, and GCG; at least a codon selected from the group consisting of CGU, CGC, CGA, CGG, AGA, and AGG; at least a codon selected from AAU or ACC; at least a codon selected from GAU or GAC; at least a codon selected from UGU or UGC; at least a codon selected from CAA or CAG; at least a codon selected from GAA or GAG; at least a codon selected from the group consisting of GGU, GGC, GGA, and GGG; at least a codon selected from CAU or CAC; at least a codon selected from the group consisting of AUU, AUC, and AUA; at least a codon selected from the group consisting of UUA, UUG, CUU, CUC, CUA, and CUG; at least a codon selected from AAA or AAG; an AUG codon; at least a codon selected from UUU or UUC; at least a codon selected from the group consisting of CCU, CCC, CCA, and CCG; at least a codon selected from the group consisting of UCU, UCC, UCA, UCG, AGU, and AGC; at least a codon selected from the group consisting of ACU, ACC, ACA, and ACG; a UGG codon; at least a codon selected from UAU or UAC; and, at least a codon selected from the group consisting of GUU, GUC, GUA, and GUG.

In some specific embodiments, the limited codon set has been optimized for in vivo expression of a sequence optimized nucleic acid (e.g., a synthetic mRNA) following administration to a certain tissue or cell.

In some embodiments, the optimized codon set (e.g., a 20 codon set encoding 20 amino acids) complies at least with one of the following properties:
the optimized codon set has a higher average G/C content than the original or native codon set; or,
the optimized codon set has a lower average U content than the original or native codon set; or,
the optimized codon set is composed of codons with the highest frequency; or,
the optimized codon set is composed of codons with the lowest frequency; or, a combination thereof.

In some specific embodiments, at least one codon in the optimized codon set has the second highest, the third highest, the fourth highest, the fifth highest or the sixth highest frequency in the synonymous codon set. In some specific embodiments, at least one codon in the optimized codon has the second lowest, the third lowest, the fourth lowest, the fifth lowest, or the sixth lowest frequency in the synonymous codon set.

As used herein, the term “native codon set” refers to the codon set used natively by the source organism to encode the reference nucleic acid sequence. As used herein, the term “original codon set” refers to the codon set used to encode the reference nucleic acid sequence before the beginning of sequence optimization, or to a codon set used to encode an optimized variant of the reference nucleic acid sequence at the beginning of a new optimization iteration when sequence optimization is applied iteratively or recursively.

In some embodiments, 5%, 10%, 15%, 20%, 25%, 30%, 35%, 40%, 45%, 50%, 55%, 60%, 65%, 70%, 75%, 80%, 85%, 90%, 95% or 100% of codons in the codon set are those with the highest frequency. In other embodiments, 5%, 10%, 15%, 20%, 25%, 30%, 35%, 40%, 45%, 50%, 55%, 60%, 65%, 70%, 75%, 80%, 85%, 90%, 95% or 100% of codons in the codon set are those with the lowest frequency.

In some embodiments, 5%, 10%, 15%, 20%, 25%, 30%, 35%, 40%, 45%, 50%, 55%, 60%, 65%, 70%, 75%, 80%, 85%, 90%, 95% or 100% of codons in the codon set are those with the highest uridine content. In some embodiments, 5%, 10%, 15%, 20%, 25%, 30%, 35%, 40%, 45%, 50%, 55%, 60%, 65%, 70%, 75%, 80%, 85%, 90%, 95% or 100% of codons in the codon set are those with the lowest uridine content.

In some embodiments, the average G/C content (absolute or relative) of the codon set is 5%, 10%, 15%, 20%, 25%, 30%, 35%, 40%, 45%, 50%, 55%, 60%, 65%, 70%, 75%, 80%, 85%, 90%, 95% or 100% higher than the average G/C content (absolute or relative) of the original codon set. In some embodiments, the average G/C content (absolute or relative) of the codon set is 5%, 10%, 15%, 20%, 25%, 30%, 35%, 40%, 45%, 50%, 55%, 60%, 65%, 70%, 75%, 80%, 85%, 90%, 95% or 100% lower than the average G/C content (absolute or relative) of the original codon set.

In some embodiments, the uracil content (absolute or relative) of the codon set is 5%, 10%, 15%, 20%, 25%, 30%, 35%, 40%, 45%, 50%, 55%, 60%, 65%, 70%, 75%, 80%, 85%, 90%, 95% or 100% higher than the average uracil content (absolute or relative) of the original codon set. In some embodiments, the uracil content (absolute or relative) of the codon set is 5%, 10%, 15%, 20%, 25%, 30%, 35%, 40%, 45%, 50%, 55%, 60%, 65%, 70%, 75%, 80%, 85%, 90%, 95% or 100% lower than the average uracil content (absolute or relative) of the original codon set. See also U.S. Appl. Publ. No. 2011/0082055, and Int'l. Publ. No. WO2000018778, both of which are incorporated herein by reference in their entireties.

### Characterization of Sequence Optimized Nucleic Acids

In some embodiments of the invention, the polynucleotide (e.g., a RNA, e.g., a mRNA) comprising a sequence optimized nucleic acid disclosed herein encoding a polypeptide (e.g., BH3) can be can be tested to determine whether at least one nucleic acid sequence property (e.g., stability when exposed to nucleases) or expression property has been improved with respect to the non-sequence optimized nucleic acid.

As used herein, “expression property” refers to a property of a nucleic acid sequence either in vivo (e.g., translation efficacy of a synthetic mRNA after administration to a subject in need thereof) or in vitro (e.g., translation efficacy of a synthetic mRNA tested in an in vitro model system). Expression properties include but are not limited to the amount of protein produced by an mRNA encoding a polypeptide (e.g., BH3) after administration, and the amount of soluble or otherwise functional protein produced. In some embodiments, sequence optimized nucleic acids disclosed herein can be evaluated according to the viability of the cells expressing a protein encoded by a sequence optimized nucleic acid sequence (e.g., a RNA, e.g., a mRNA) encoding a polypeptide (e.g., BH3) disclosed herein.

In a particular embodiment, a plurality of sequence optimized nucleic acids disclosed herein (e.g., a RNA, e.g., a mRNA) containing codon substitutions with respect to the non-optimized reference nucleic acid sequence can be characterized functionally to measure a property of interest, for example an expression property in an in vitro model system, or in vivo in a target tissue or cell.

a. Optimization of Nucleic Acid Sequence Intrinsic Properties

In some embodiments of the invention, the desired property of the polynucleotide is an intrinsic property of the nucleic acid sequence. For example, the nucleotide sequence (e.g., a RNA, e.g., a mRNA) can be sequence optimized for in vivo or in vitro stability. In some embodiments, the nucleotide sequence can be sequence optimized for expression in a particular target tissue or cell. In some embodiments, the nucleic acid sequence is sequence optimized to increase its plasma half by preventing its degradation by endo and exonucleases.

In other embodiments, the nucleic acid sequence is sequence optimized to increase its resistance to hydrolysis in solution, for example, to lengthen the time that the sequence optimized nucleic acid or a pharmaceutical composition comprising the sequence optimized nucleic acid can be stored under aqueous conditions with minimal degradation.

In other embodiments, the sequence optimized nucleic acid can be optimized to increase its resistance to hydrolysis in dry storage conditions, for example, to lengthen the time that the sequence optimized nucleic acid can be stored after lyophilization with minimal degradation.

b. Nucleic Acids Sequence Optimized for Protein Expression

In some embodiments of the invention, the desired property of the polynucleotide is the level of expression of a polypeptide (e.g., BH3) encoded by a sequence optimized sequence disclosed herein. Protein expression levels can be measured using one or more expression systems. In some embodiments, expression can be measured in cell culture systems, e.g., CHO cells or HEK293 cells. In some embodiments, expression can be measured using in vitro expression systems prepared from extracts of living cells, e.g., rabbit reticulocyte lysates, or in vitro expression systems prepared by assembly of purified individual components. In other embodiments, the protein expression is measured in an in vivo system, e.g., mouse, rabbit, monkey, etc.

In some embodiments, protein expression in solution form can be desirable. Accordingly, in some embodiments, a reference sequence can be sequence optimized to yield a sequence optimized nucleic acid sequence having optimized levels of expressed proteins in soluble form. Levels of protein expression and other properties such as solubility, levels of aggregation, and the presence of truncation products (i.e., fragments due to proteolysis, hydrolysis, or defective translation) can be measured according to methods known in the art, for example, using electrophoresis (e.g., native or SDS-PAGE) or chromatographic methods (e.g., HPLC, size exclusion chromatography, etc.).

c. Optimization of Target Tissue or Target Cell Viability

In some embodiments, the expression of heterologous therapeutic proteins encoded by a nucleic acid sequence can have deleterious effects in the target tissue or cell, reducing protein yield, or reducing the quality of the expressed product (e.g., due to the presence of protein fragments or precipitation of the expressed protein in inclusion bodies), or causing toxicity.

Accordingly, in some embodiments of the invention, the sequence optimization of a nucleic acid sequence disclosed herein, e.g., a nucleic acid sequence encoding a polypeptide (e.g., BH3), can be used to increase the viability of target cells expressing the protein encoded by the sequence optimized nucleic acid.

Heterologous protein expression can also be deleterious to cells transfected with a nucleic acid sequence for autologous or heterologous transplantation. Accordingly, in some embodiments of the present disclosure the sequence optimization of a nucleic acid sequence disclosed herein can be used to increase the viability of target cells expressing the protein encoded by the sequence optimized nucleic acid sequence. Changes in cell or tissue viability, toxicity, and other physiological reaction can be measured according to methods known in the art.

d. Reduction of Immune and/or Inflammatory Response

In some cases, the administration of a sequence optimized nucleic acid encoding a polypeptide (e.g., BH3) or a functional fragment thereof can trigger an immune response, which could be caused by (i) the therapeutic agent (e.g., an mRNA encoding a BH3 polypeptide), or (ii) the expression product of such therapeutic agent (e.g., the BH3 polypeptide encoded by the mRNA), or (iv) a combination thereof. Accordingly, in some embodiments of the present disclosure the sequence optimization of nucleic acid sequence (e.g., an mRNA) disclosed herein can be used to decrease an immune or inflammatory response triggered by the administration of a nucleic acid encoding a polypeptide (e.g., BH3) or by the expression product of a polypeptide (e.g., BH3) encoded by such nucleic acid.

In some aspects, an inflammatory response can be measured by detecting increased levels of one or more inflammatory cytokines using methods known in the art, e.g., ELISA. The term “inflammatory cytokine” refers to cytokines that are elevated in an inflammatory response. Examples of inflammatory cytokines include interleukin-6 (IL-6), CXCL1 (chemokine (C-X-C motif) ligand 1; also known as GROα, interferon-γ (IFNγ), tumor necrosis factor α (TNFα), interferon γ-induced protein 10 (IP-10), or granulocyte-colony stimulating factor (G-CSF). The term inflammatory cytokines includes also other cytokines associated with inflammatory responses known in the art, e.g., interleukin-1 (IL-1), interleukin-8 (IL-8), interleukin-12 (IL-12), interleukin-13 (Il-13), interferon α (IFN-α), etc.

### Modified Nucleotide Sequences Encoding Polypeptides

In some embodiments, the polynucleotide of the invention (e.g., a RNA, e.g., a mRNA) comprises a chemically modified nucleobase, e.g., 5-methoxyuracil. In some embodiments, the mRNA is a uracil-modified sequence comprising an ORF encoding a polypeptide described herein (e.g., BH3), wherein the mRNA comprises a chemically modified nucleobase, e.g., 5-methoxyuracil.

In certain aspects of the invention, when the 5-methoxyuracil base is connected to a ribose sugar, as it is in polynucleotides, the resulting modified nucleoside or nucleotide is referred to as 5-methoxyuridine. In some embodiments, uracil in the polynucleotide is at least about 25%, at least about 30%, at least about 40%, at least about 50%, at least about 60%, at least about 70%, at least about 80%, at least 90%, at least 95%, at least 99%, or about 100% 5-methoxyuracil. In one embodiment, uracil in the polynucleotide is at least 95% 5-methoxyuracil. In another embodiment, uracil in the polynucleotide is 100% 5-methoxyuracil.

In embodiments where uracil in the polynucleotide is at least 95% 5-methoxyuracil, overall uracil content can be adjusted such that the polynucleotide of the invention (e.g., a RNA, e.g., a mRNA) provides suitable protein expression levels while inducing little to no immune response.

In some embodiments, the uracil content of the ORF is between about 105% and about 145%, about 105% and about 140%, about 110% and about 140%, about 110% and about 145%, about 115% and about 135%, about 105% and about 135%, about 110% and about 135%, about 115% and about 145%, or about 115% and about 140% of the theoretical minimum uracil content in the corresponding wild-type ORF (% U_{TM}). In other embodiments, the uracil content of the ORF is between about 117% and about 134% or between 118% and 132% of the % U_{TM}. In some embodiments, the uracil content of the ORF encoding a polypeptide (e.g., BH3) is about 115%, about 120%, about 125%, about 130%, about 135%, about 140%, about 145%, or about 150% of the % U_{TM}. In this context, the term “uracil” can refer to 5-methoxyuracil and/or naturally occurring uracil.

In some embodiments, the uracil content in the ORF of the mRNA encoding a polypeptide of the invention (e.g., BH3) is less than about 50%, about 40%, about 30%, or about 20% of the total nucleobase content in the ORF. In some embodiments, the uracil content in the ORF is between about 15% and about 25% of the total nucleobase content in the ORF. In other embodiments, the uracil content in the ORF is between about 20% and about 30% of the total nucleobase content in the ORF. In one embodiment, the uracil content in the ORF of the mRNA encoding a polypeptide (e.g., BH3) is less than about 20% of the total nucleobase content in the open reading frame. In this context, the term “uracil” can refer to 5-methoxyuracil and/or naturally occurring uracil.

In further embodiments, the ORF of the mRNA encoding a polypeptide (e.g., BH3) having 5-methoxyuracil and adjusted uracil content has increased Cytosine (C), Guanine (G), or Guanine/Cytosine (G/C) content (absolute or relative). In some embodiments, the overall increase in C, G, or G/C content (absolute or relative) of the ORF is at least about 2%, at least about 3%, at least about 4%, at least about 5%, at least about 6%, at least about 7%, at least about 10%, at least about 15%, at least about 20%, at least about 40%, at least about 50%, at least about 60%, at least about 70%, at least about 80%, at least about 90%, at least about 95%, or at least about 100% relative to the G/C content (absolute or relative) of the wild-type ORF. In some embodiments, the G, the C, or the G/C content in the ORF is less than about 100%, less than about 90%, less than about 85%, or less than about 80% of the theoretical maximum G, C, or G/C content of the corresponding wild type nucleotide sequence encoding the polypeptide (e.g., BH3) (% G_{TMX}; % C_{TMX}, or % G/C_{TMX}). In other embodiments, the G, the C, or the G/C content in the ORF is between about 70% and about 80%, between about 71% and about 79%, between about 71% and about 78%, or between about 71% and about 77% of the % G_{TMX}, % C_{TMX}, or % G/C_{TMX}. In some embodiments, the increases in G and/or C content (absolute or relative) described herein can be conducted by replacing synonymous codons with low G, C, or G/C content with synonymous codons having higher G, C, or G/C content. In other embodiments, the increase in G and/or C content (absolute or relative) is conducted by replacing a codon ending with U with a synonymous codon ending with G or C.

In further embodiments, the ORF of the mRNA encoding a polypeptide of the invention (e.g., BH3) comprises 5-methoxyuracil and has an adjusted uracil content containing less uracil pairs (UU) and/or uracil triplets (UUU) and/or uracil quadruplets (UUUU) than the corresponding wild-type nucleotide sequence encoding the polypeptide (e.g., BH3). In some embodiments, the ORF of the mRNA encoding a polypeptide of the invention (e.g., BH3) contains no uracil pairs and/or uracil triplets and/or uracil quadruplets. In some embodiments, uracil pairs and/or uracil triplets and/or uracil quadruplets are reduced below a certain threshold, e.g., no more than 1, 2, 3, 4, 5, 6, 7, 8, 9, 10, 11, 12, 13, 14, 15, 16, 17, 18, 19, or 20 occurrences in the ORF of the mRNA encoding the polypeptide (e.g., BH3). In a particular embodiment, the ORF of the mRNA encoding the polypeptide of the invention (e.g., BH3) contains less than 20, 19, 18, 17, 16, 15, 14, 13, 12, 11, 10, 9, 8, 7, 6, 5, 4, 3, 2, or 1 non-phenylalanine uracil pairs and/or triplets. In another embodiment, the ORF of the mRNA encoding the polypeptide (e.g., BH3) contains no non-phenylalanine uracil pairs and/or triplets.

In further embodiments, the ORF of the mRNA encoding a polypeptide of the invention (e.g., BH3) comprises 5-methoxyuracil and has an adjusted uracil content containing less uracil-rich clusters than the corresponding wild-type nucleotide sequence encoding the polypeptide. In some embodiments, the ORF of the mRNA encoding the polypeptide of the invention (e.g., BH3) contains uracil-rich clusters that are shorter in length than corresponding uracil-rich clusters in the corresponding wild-type nucleotide sequence encoding the polypeptide.

In further embodiments, alternative lower frequency codons are employed. At least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the codons in the polypeptide (e.g., BH3)-encoding ORF of the 5-methoxyuracil-comprising mRNA are substituted with alternative codons, each alternative codon having a codon frequency lower than the codon frequency of the substituted codon in the synonymous codon set. The ORF also has adjusted uracil content, as described above. In some embodiments, at least one codon in the ORF of the mRNA encoding the polypeptide (e.g., BH3) is substituted with an alternative codon having a codon frequency lower than the codon frequency of the substituted codon in the synonymous codon set.

In some embodiments, the adjusted uracil content, polypeptide (e.g., BH3)-encoding ORF of the 5-methoxyuracil-comprising mRNA exhibits expression levels of the polypeptide when administered to a mammalian cell that are higher than expression levels of the polypeptide from the corresponding wild-type mRNA. In other embodiments, the expression levels of the polypeptide (e.g., BH3) when administered to a mammalian cell are increased relative to a corresponding mRNA containing at least 95% 5-methoxyuracil and having a uracil content of about 160%, about 170%, about 180%, about 190%, or about 200% of the theoretical minimum. In yet other embodiments, the expression levels of the polypeptide (e.g., BH3) when administered to a mammalian cell are increased relative to a corresponding mRNA, wherein at least about 50%, at least about 60%, at least about 70%, at least about 80%, at least about 90%, or about 100% of uracils are 1-methylpseudouracil or pseudouracils. In some embodiments, the mammalian cell is a mouse cell, a rat cell, or a rabbit cell. In other embodiments, the mammalian cell is a monkey cell or a human cell. In some embodiments, the human cell is a HeLa cell, a BJ fibroblast cell, or a peripheral blood mononuclear cell (PBMC). In some embodiments, the polypeptide (e.g., BH3) is expressed when the mRNA is administered to a mammalian cell in vivo. In some embodiments, the mRNA is administered to mice, rabbits, rats, monkeys, or humans. In one embodiment, mice are null mice. In some embodiments, the mRNA is administered to mice in an amount of about 0.01 mg/kg, about 0.05 mg/kg, about 0.1 mg/kg, or about 0.15 mg/kg. In some embodiments, the mRNA is administered intravenously or intramuscularly. In other embodiments, the polypeptide (e.g., BH3) is expressed when the mRNA is administered to a mammalian cell in vitro. In some embodiments, the expression is increased by at least about 2-fold, at least about 5-fold, at least about 10-fold, at least about 50-fold, at least about 500-fold, at least about 1500-fold, or at least about 3000-fold. In other embodiments, the expression is increased by at least about 10%, about 20%, about 30%, about 40%, about 50%, 60%, about 70%, about 80%, about 90%, or about 100%.

In some embodiments, adjusted uracil content, polypeptide (e.g., BH3)-encoding ORF of the 5-methoxyuracil-comprising mRNA exhibits increased stability. In some embodiments, the mRNA exhibits increased stability in a cell relative to the stability of a corresponding wild-type mRNA under the same conditions. In some embodiments, the mRNA exhibits increased stability including resistance to nucleases, thermal stability, and/or increased stabilization of secondary structure. In some embodiments, increased stability exhibited by the mRNA is measured by determining the half-life of the mRNA (e.g., in a plasma, cell, or tissue sample) and/or determining the area under the curve (AUC) of the protein expression by the mRNA over time (e.g., in vitro or in vivo). An mRNA is identified as having increased stability if the half-life and/or the AUC is greater than the half-life and/or the AUC of a corresponding wild-type mRNA under the same conditions.

In some embodiments, the mRNA of the present invention induces a detectably lower immune response (e.g., innate or acquired) relative to the immune response induced by a corresponding wild-type mRNA under the same conditions. In other embodiments, the mRNA of the present disclosure induces a detectably lower immune response (e.g., innate or acquired) relative to the immune response induced by an mRNA that encodes for a polypeptide (e.g., BH3) but does not comprise 5-methoxyuracil under the same conditions, or relative to the immune response induced by an mRNA that encodes for a polypeptide (e.g., BH3) and that comprises 5-methoxyuracil but that does not have adjusted uracil content under the same conditions. The innate immune response can be manifested by increased expression of pro-inflammatory cytokines, activation of intracellular PRRs (RIG-I, MDA5, etc), cell death, and/or termination or reduction in protein translation. In some embodiments, a reduction in the innate immune response can be measured by expression or activity level of Type 1 interferons (e.g., IFN-α, IFN-β, IFN-κ, IFN-δ, IFN-ε, IFN-τ, IFN-ω, and IFN-ζ) or the expression of interferon-regulated genes such as the toll-like receptors (e.g., TLR7 and TLR8), and/or by decreased cell death following one or more administrations of the mRNA of the invention into a cell.

In some embodiments, the expression of Type-1 interferons by a mammalian cell in response to the mRNA of the present disclosure is reduced by at least 10%, 20%, 30%, 40%, 50%, 60%, 70%, 80%, 90%, 95%, 99%, 99.9%, or greater than 99.9% relative to a corresponding wild-type mRNA, to an mRNA that encodes a polypeptide (e.g., BH3) but does not comprise 5-methoxyuracil, or to an mRNA that encodes a polypeptide (e.g., BH3) and that comprises 5-methoxyuracil but that does not have adjusted uracil content. In some embodiments, the interferon is IFN-β. In some embodiments, cell death frequency cased by administration of mRNA of the present disclosure to a mammalian cell is 10%, 25%, 50%, 75%, 85%, 90%, 95%, or over 95% less than the cell death frequency observed with a corresponding wild-type mRNA, an mRNA that encodes for a polypeptide (e.g., BH3) but does not comprise 5-methoxyuracil, or an mRNA that encodes for a polypeptide (e.g., BH3) and that comprises 5-methoxyuracil but that does not have adjusted uracil content. In some embodiments, the mammalian cell is a BJ fibroblast cell. In other embodiments, the mammalian cell is a splenocyte. In some embodiments, the mammalian cell is that of a mouse or a rat. In other embodiments, the mammalian cell is that of a human. In one embodiment, the mRNA of the present disclosure does not substantially induce an innate immune response of a mammalian cell into which the mRNA is introduced.

In some embodiments, the polynucleotide is an mRNA that comprises an ORF that encodes a polypeptide (e.g., BH3), wherein uracil in the mRNA is at least about 95% 5-methoxyuracil, wherein the uracil content of the ORF is between about 115% and about 135% of the theoretical minimum uracil content in the corresponding wild-type ORF, and wherein the uracil content in the ORF encoding the polypeptide (e.g., BH3) is less than about 30% of the total nucleobase content in the ORF. In some embodiments, the ORF that encodes the polypeptide (e.g., BH3) is further modified to increase G/C content of the ORF (absolute or relative) by at least about 40%, as compared to the corresponding wild-type ORF. In yet other embodiment, the ORF encoding the polypeptide (e.g., BH3) contains less than 20 non-phenylalanine uracil pairs and/or triplets. In some embodiments, at least one codon in the ORF of the mRNA encoding the polypeptide (e.g., BH3) is further substituted with an alternative codon having a codon frequency lower than the codon frequency of the substituted codon in the synonymous codon set. In some embodiments, the expression of the polypeptide (e.g., BH3) encoded by an mRNA comprising an ORF wherein uracil in the mRNA is at least about 95% 5-methoxyuracil, and wherein the uracil content of the ORF is between about 115% and about 135% of the theoretical minimum uracil content in the corresponding wild-type ORF, is increased by at least about 10-fold when compared to expression of the polypeptide (e.g., BH3) from the corresponding wild-type mRNA. In some embodiments, the mRNA comprises an open ORF wherein uracil in the mRNA is at least about 95% 5-methoxyuracil, and wherein the uracil content of the ORF is between about 115% and about 135% of the theoretical minimum uracil content in the corresponding wild-type ORF, and wherein the mRNA does not substantially induce an innate immune response of a mammalian cell into which the mRNA is introduced.

### Methods for Modifying Polynucleotides

The invention includes modified polynucleotides comprising a polynucleotide described herein (e.g., a polynucleotide comprising a nucleotide sequence encoding a BH3 polypeptide). The modified polynucleotides can be chemically modified and/or structurally modified. When the polynucleotides of the present invention are chemically and/or structurally modified the polynucleotides can be referred to as “modified polynucleotides.”

The present disclosure provides for modified nucleosides and nucleotides of a polynucleotide (e.g., RNA polynucleotides, such as mRNA polynucleotides) encoding a BH3 polypeptide. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can be synthesized by any useful method, such as, for example, chemically, enzymatically, or recombinantly, to include one or more modified or non-natural nucleosides. Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In some embodiments, the modified polynucleotides contain one, two, or more (optionally different) nucleoside or nucleotide modifications. In some embodiments, a modified polynucleotide, introduced to a cell can exhibit one or more desirable properties, e.g., improved protein expression, reduced immunogenicity, or reduced degradation in the cell, as compared to an unmodified polynucleotide.

a. Structural Modifications

In some embodiments, a polynucleotide of the present invention (e.g., a polynucleotide comprising a nucleotide sequence encoding a BH3 polypeptide) is structurally modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” can be chemically modified to “AT-5meC-G”. The same polynucleotide can be structurally modified from “ATCG” to “ATCCCG”. Here, the dinucleotide “CC” has been inserted, resulting in a structural modification to the polynucleotide.

b. Chemical Modifications

In some embodiments, the polynucleotides of the present invention (e.g., a polynucleotide comprising a nucleotide sequence encoding a BH3 polypeptide) are chemically modified. As used herein in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof. Generally, herein, these terms are not intended to refer to the ribonucleotide modifications in naturally occurring 5′-terminal mRNA cap moieties.

In some embodiments, the polynucleotides of the invention (e.g., a polynucleotide comprising a nucleotide sequence encoding a BH3 polypeptide) can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation, such as where all uridines are replaced by a uridine analog, e.g., 5-methoxyuridine. In another embodiment, the polynucleotides can have a uniform chemical modification of two, three, or four of the same nucleoside type throughout the entire polynucleotide (such as all uridines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s.

Modifications of polynucleotides (e.g., RNA polynucleotides, such as mRNA polynucleotides) that are useful in the compositions, methods and synthetic processes of the present disclosure include, but are not limited to the following nucleotides, nucleosides and nucleobases: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-α-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-α-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-ethyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methyluridine,), 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uracil; N1-ethyl-pseudo-uracil; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; α-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 1-Ethyl-pseudo-UTP; 2 (thio)pseudouracil; 2′ deoxy uridine; 2′ fluorouridine; 2-(thio)uracil; 2,4-(dithio)pseudouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromouridine; 5-iodo-uridine; 5-uracil; 6 (azo)uracil; 6-(azo)uracil; 6-aza-uridine; allyamino-uracil; aza uracil; deaza uracil; N3 (methyl)uracil; P seudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl} pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-α-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}] propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine;1-(aza)-2-(thio)-3-(aza)-phenoxazin-1-yl: 1,3-(diaza)-2-(oxo)-phenthiazin-1-yl;1,3-(diaza)-2-(oxo)-phenoxazin-1-yl;1,3,5-(triaza)-2,6-(dioxa)-naphthalene;2 (amino)purine;2,4,5-(trimethyl)phenyl;2′ methyl, 2′amino, 2′azido, 2′fluro-cytidine;2′ methyl, 2′amino, 2′azido, 2′fluro-adenine;2′methyl, 2′amino, 2′azido, 2′fluro-uridine;2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, the polynucleotide (e.g., RNA polynucleotide, such as mRNA polynucleotide) includes a combination of at least two (e.g., 2, 3, 4 or more) of the aforementioned modified nucleobases.

In some embodiments, the mRNA comprises at least one chemically modified nucleoside. In some embodiments, the at least one chemically modified nucleoside is selected from the group consisting of pseudouridine (ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1ψ), 1-ethyl-pseudouridine (e1ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, 2,8-dimethyladenosine, 2-geranylthiouridine, 2-lysidine, 2-selenouridine, 3-(3-amino-3-carboxypropyl)-5,6-dihydrouridine, 3-(3-amino-3-carboxypropyl)pseudouridine, 3-methylpseudouridine, 5-(carboxyhydroxymethyl)-2′-O-methyluridine methyl ester, 5-aminomethyl-2-geranylthiouridine, 5-aminomethyl-2-selenouridine, 5-aminomethyluridine, 5-carbamoylhydroxymethyluridine, 5-carbamoylmethyl-2-thiouridine, 5-carboxymethyl-2-thiouridine, 5-carboxymethylaminomethyl-2-geranylthiouridine, 5-carboxymethylaminomethyl-2-selenouridine, 5-cyanomethyluridine, 5-hydroxycytidine, 5-methylaminomethyl-2-geranylthiouridine, 7-aminocarboxypropyl-demethylwyosine, 7-aminocarboxypropylwyosine, 7-aminocarboxypropylwyosine methyl ester, 8-methyladenosine, N4,N4-dimethylcytidine, N6-formyladenosine, N6-hydroxymethyladenosine, agmatidine, cyclic N6-threonylcarbamoyladenosine, glutamyl-queuosine, methylated undermodified hydroxywybutosine, N4,N4,2′-O-trimethylcytidine, geranylated 5-methylaminomethyl-2-thiouridine, geranylated 5-carboxymethylaminomethyl-2-thiouridine, Qbase, preQ0base, preQ1base, and two or more combinations thereof. In some embodiments, the at least one chemically modified nucleoside is selected from the group consisting of pseudouridine, 1-methyl-pseudouridine, 1-ethyl-pseudouridine, 5-methylcytosine, 5-methoxyuridine, and a combination thereof. In some embodiments, the polynucleotide (e.g., RNA polynucleotide, such as mRNA polynucleotide) includes a combination of at least two (e.g., 2, 3, 4 or more) of the aforementioned modified nucleobases.

### (i) Base Modifications

In certain embodiments, the chemical modification is at nucleobases in the polynucleotides (e.g., RNA polynucleotide, such as mRNA polynucleotide). In some embodiments, modified nucleobases in the polynucleotide (e.g., RNA polynucleotide, such as mRNA polynucleotide) are selected from the group consisting of 1-methyl-pseudouridine (m1ψ), 1-ethyl-pseudouridine (e1ψ), 5-methoxy-uridine (mo5U), 5-methyl-cytidine (m5C), pseudouridine (ψ), α-thio-guanosine and α-thio-adenosine. In some embodiments, the polynucleotide includes a combination of at least two (e.g., 2, 3, 4 or more) of the aforementioned modified nucleobases.

In some embodiments, the polynucleotide (e.g., RNA polynucleotide, such as mRNA polynucleotide) comprises pseudouridine (ψ) and 5-methyl-cytidine (m5C). In some embodiments, the polynucleotide (e.g., RNA polynucleotide, such as mRNA polynucleotide) comprises 1-methyl-pseudouridine (m1ψ). In some embodiments, the polynucleotide (e.g., RNA polynucleotide, such as mRNA polynucleotide) comprises 1-ethyl-pseudouridine (e1ψ). In some embodiments, the polynucleotide (e.g., RNA polynucleotide, such as mRNA polynucleotide) comprises 1-methyl-pseudouridine (m1ψ) and 5-methyl-cytidine (m5C). In some embodiments, the polynucleotide (e.g., RNA polynucleotide, such as mRNA polynucleotide) comprises 1-ethyl-pseudouridine (e1ψ) and 5-methyl-cytidine (m5C). In some embodiments, the polynucleotide (e.g., RNA polynucleotide, such as mRNA polynucleotide) comprises 2-thiouridine (s2U). In some embodiments, the polynucleotide (e.g., RNA polynucleotide, such as mRNA polynucleotide) comprises 2-thiouridine and 5-methyl-cytidine (m5C). In some embodiments, the polynucleotide (e.g., RNA polynucleotide, such as mRNA polynucleotide) comprises methoxy-uridine (mo5U). In some embodiments, the polynucleotide (e.g., RNA polynucleotide, such as mRNA polynucleotide) comprises 5-methoxy-uridine (mo5U) and 5-methyl-cytidine (m5C). In some embodiments, the polynucleotide (e.g., RNA polynucleotide, such as mRNA polynucleotide) comprises 2′-O-methyl uridine. In some embodiments, the polynucleotide (e.g., RNA polynucleotide, such as mRNA polynucleotide) comprises 2′-O-methyl uridine and 5-methyl-cytidine (m5C). In some embodiments, the polynucleotide (e.g., RNA polynucleotide, such as mRNA polynucleotide) comprises N6-methyl-adenosine (m6A). In some embodiments, the polynucleotide (e.g., RNA polynucleotide, such as mRNA polynucleotide) comprises N6-methyl-adenosine (m6A) and 5-methyl-cytidine (m5C).

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as any of those set forth above. In some embodiments, the chemically modified nucleosides in the open reading frame are selected from the group consisting of uridine, adenine, cytosine, guanine, and any combination thereof.

In some embodiments, the modified nucleobase is a modified cytosine. Examples of nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine.

In some embodiments, a modified nucleobase is a modified uridine. Example nucleobases and nucleosides having a modified uridine include 5-cyano uridine or 4′-thio uridine.

In some embodiments, a modified nucleobase is a modified adenine. Example nucleobases and nucleosides having a modified adenine include 7-deaza-adenine, 1-methyl-adenosine (m1A), 2-methyl-adenine (m2A), N6-methyl-adenine (m6A), and 2,6-Diaminopurine.

In some embodiments, a modified nucleobase is a modified guanine. Example nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

In some embodiments, the nucleobase modified nucleotides in the polynucleotide (e.g., RNA polynucleotide, such as mRNA polynucleotide) are 5-methoxyuridine.

In some embodiments, the polynucleotide (e.g., RNA polynucleotide, such as mRNA polynucleotide) includes a combination of at least two (e.g., 2, 3, 4 or more) of modified nucleobases.

In some embodiments, at least 95% of a type of nucleobases (e.g., uracil) in a polynucleotide of the invention (e.g., an mRNA polynucleotide encoding BH3) are modified nucleobases. In some embodiments, at least 95% of uracil in a polynucleotide of the present invention (e.g., an mRNA polynucleotide encoding BH3) is 5-methoxyuracil.

In some embodiments, the polynucleotide (e.g., RNA polynucleotide, such as mRNA polynucleotide) comprises 5-methoxyuridine (5mo5U) and 5-methyl-cytidine (m5C).

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 5-methoxyuridine, meaning that substantially all uridine residues in the mRNA sequence are replaced with 5-methoxyuridine. Similarly, a polynucleotide can be uniformly modified for any type of nucleoside residue present in the sequence by replacement with a modified residue such as any of those set forth above.

In some embodiments, the modified nucleobase is a modified cytosine.

In some embodiments, a modified nucleobase is a modified uracil. Example nucleobases and nucleosides having a modified uracil include 5-methoxyuracil.

In some embodiments, a modified nucleobase is a modified adenine.

In some embodiments, a modified nucleobase is a modified guanine.

In some embodiments, the nucleobases, sugar, backbone, or any combination thereof in the open reading frame encoding a polypeptide (e.g., BH3) are chemically modified by at least 10%, at least 20%, at least 30%, at least 40%, at least 50%, at least 60%, at least 70%, at least 80%, at least 90%, at least 95%, at least 99%, or 100%.

In some embodiments, the uridine nucleosides in the open reading frame encoding a polypeptide (e.g., BH3) are chemically modified by at least 10%, at least 20%, at least 30%, at least 40%, at least 50%, at least 60%, at least 70%, at least 80%, at least 90%, at least 95%, at least 99%, or 100%.

In some embodiments, the adenosine nucleosides in the open reading frame encoding a polypeptide (e.g., BH3) are chemically modified by at least 10%, at least 20%, at least 30%, at least 40%, at least 50%, at least 60%, at least 70%, at least 80%, at least 90%, at least 95%, at least 99%, or 100%.

In some embodiments, the cytidine nucleosides in the open reading frame encoding a polypeptide (e.g., BH3) are chemically modified by at least at least 10%, at least 20%, at least 30%, at least 40%, at least 50%, at least 60%, at least 70%, at least 80%, at least 90%, at least 95%, at least 99%, or 100%.

In some embodiments, the guanosine nucleosides in the open reading frame encoding a polypeptide (e.g., BH3) are chemically modified by at least at least 10%, at least 20%, at least 30%, at least 40%, at least 50%, at least 60%, at least 70%, at least 80%, at least 90%, at least 95%, at least 99%, or 100%.

In some embodiments, the polynucleotides can include any useful linker between the nucleosides. Such linkers, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

### Untranslated Regions (UTRs)

Untranslated regions (UTRs) are nucleic acid sections of a polynucleotide before a start codon (5′UTR) and after a stop codon (3′UTR) that are not translated. In some embodiments, a polynucleotide (e.g., a ribonucleic acid (RNA), e.g., a messenger RNA (mRNA)) of the invention comprising an open reading frame (ORF) encoding a polypeptide (e.g., BH3) further comprises UTR (e.g., a 5′UTR or functional fragment thereof, a 3′UTR or functional fragment thereof, or a combination thereof).

A UTR can be homologous or heterologous to the coding region in a polynucleotide. In some embodiments, the UTR is homologous to the ORF encoding the polypeptide (e.g., BH3). In some embodiments, the UTR is heterologous to the ORF encoding the polypeptide (e.g., BH3). In some embodiments, the polynucleotide comprises two or more 5′UTRs or functional fragments thereof, each of which have the same or different nucleotide sequences. In some embodiments, the polynucleotide comprises two or more 3′UTRs or functional fragments thereof, each of which have the same or different nucleotide sequences.

In some embodiments, the 5′UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′UTR or 3′UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTRs also have been known to form secondary structures that are involved in elongation factor binding.

By engineering the features typically found in abundantly expressed genes of specific target organs, one can enhance the stability and protein production of a polynucleotide. For example, introduction of 5′UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′UTR and the 3′UTR can be heterologous. In some embodiments, the 5′UTR can be derived from a different species than the 3′UTR. In some embodiments, the 3′UTR can be derived from a different species than the 5′UTR. Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the Rsubunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

Other exemplary 5′ and 3′ UTRs include, but are not limited to, those described in Karikóet al., Mol. Ther. 2008 16(11):1833-1840; Karikó et al., Mol. Ther. 2012 20(5):948-953; Karikó et al., Nucleic Acids Res. 2011 39(21):e142; Strong et al., Gene Therapy 1997 4:624-627; Hansson et al., J. Biol. Chem. 2015 290(9):5661-5672; Yu et al., Vaccine 2007 25(10):1701-1711; Cafri et al., Mol. Ther. 2015 23(8):1391-1400; Andries et al., Mol. Pharm. 2012 9(8):2136-2145; Crowley et al., Gene Ther. 2015 Jun. 30, doi:10.1038/gt.2015.68; Ramunas et al., FASEB J. 2015 29(5):1930-1939; Wang et al., Curr. Gene Ther. 2015 15(4):428-435; Holtkamp et al., Blood 2006 108(13):4009-4017; Kormann et al., Nat. Biotechnol. 201129(2):154-157; Poleganov et al., Hum. Gen. Ther. 2015 26(11):751-766; Warren et al., Cell Stem Cell 2010 7(5):618-630; Mandal and Rossi, Nat. Protoc. 2013 8(3):568-582; Holcik and Liebhaber, PNAS 1997 94(6):2410-2414; Ferizi et al., Lab Chip. 2015 15(17):3561-3571; Thess et al., Mol. Ther. 2015 23(9):1456-1464; Boros et al., PLoS One 2015 10(6):e0131141; Boros et al., J. Photochem. Photobiol. B. 2013 129:93-99; Andries et al., J. Control. Release 2015 217:337-344; Zinckgraf et al., Vaccine 2003 21(15):1640-9; Garneau et al., J. Virol. 2008 82(2):880-892; Holden and Harris, Virology 2004 329(1):119-133; Chiu et al., J. Virol. 2005 79(13):8303-8315; Wang et al., EMBO J. 1997 16(13):4107-4116; Al-Zoghaibi et al., Gene 2007 391(1-2):130-9; Vivinus et al., Eur. J. Biochem. 2001268(7):1908-1917; Gan and Rhoads, J. Biol. Chem. 1996 271(2):623-626; Boado et al., J. Neurochem. 1996 67(4):1335-1343; Knirsch and Clerch, Biochem. Biophys. Res. Commun. 2000 272(1):164-168; Chung et al., Biochemistry 1998 37(46):16298-16306; Izquierdo and Cuevza, Biochem. J. 2000 346 Pt 3:849-855; Dwyer et al., J. Neurochem. 1996 66(2):449-458; Black et al., Mol. Cell. Biol. 1997 17(5):2756-2763; Izquierdo and Cuevza, Mol. Cell. Biol. 1997 17(9):5255-5268; U.S. Pat. Nos. 8,278,036; 8,748,089; 8,835,108; 9,012,219; US2010/0129877; US2011/0065103; US2011/0086904; US2012/0195936; US2014/020675; US2013/0195967; US2014/029490; US2014/0206753; WO2007/036366; WO2011/015347; WO2012/072096; WO2013/143555; WO2014/071963; WO2013/185067; WO2013/182623; WO2014/089486; WO2013/185069; WO2014/144196; WO2014/152659; 2014/152673; WO2014/152940; WO2014/152774; WO2014/153052; WO2014/152966, WO2014/152513; WO2015/101414; WO2015/101415; WO2015/062738; and WO2015/024667; the contents of each of which are incorporated herein by reference in their entirety.

In some embodiments, the 5′UTR is selected from the group consisting of a 3-globin 5′UTR; a 5′UTR containing a strong Kozak translational initiation signal; a cytochrome b-245 α polypeptide (CYBA) 5′UTR; a hydroxysteroid (17-β) dehydrogenase (HSD17B4) 5′UTR; a Tobacco etch virus (TEV) 5′UTR; a Venezuelen equine encephalitis virus (TEEV) 5′UTR; a 5′ proximal open reading frame of rubella virus (RV) RNA encoding nonstructural proteins; a Dengue virus (DEN) 5′UTR; a heat shock protein 70 (Hsp70) 5′UTR; a eIF4G 5′UTR; a GLUT1 5′UTR; functional fragments thereof and any combination thereof.

In some embodiments, the 3′UTR is selected from the group consisting of a β-globin 3′UTR; a CYBA 3′UTR; an albumin 3′UTR; a growth hormone (GH) 3′UTR; a VEEV 3′UTR; a hepatitis B virus (HBV) 3′UTR; α-globin 3′UTR; a DEN 3′UTR; a PAV barley yellow dwarf virus (BYDV-PAV) 3′UTR; an elongation factor 1 α1 (EEF1A1) 3′UTR; a manganese superoxide dismutase (MnSOD) 3′UTR; a β subunit of mitochondrial H(+)-ATP synthase (β-mRNA) 3′UTR; a GLUT1 3′UTR; a MEF2A 3′UTR; a β-F1-ATPase 3′UTR; functional fragments thereof and combinations thereof.

Other exemplary UTRs include, but are not limited to, one or more of the UTRs, including any combination of UTRs, disclosed in WO2014/164253, the contents of which are incorporated herein by reference in their entirety. Shown in Table 21 of U.S. Provisional Application No. 61/775,509 and in Table 22 of U.S. Provisional Application No. 61/829,372, the contents of each are incorporated herein by reference in their entirety, is a listing start and stop sites for 5′UTRs and 3′UTRs. In Table 21, each 5′UTR (5′-UTR-005 to 5′-UTR 68511) is identified by its start and stop site relative to its native or wild-type (homologous) transcript (ENST; the identifier used in the ENSEMBL database).

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety, and sequences available at www.addgene.org/Derrick_Rossi/, last accessed Apr. 16, 2016. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′UTR or 3′UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the polynucleotides of the invention comprise a 5′UTR and/or a 3′UTR selected from any of the UTRs disclosed herein. In some embodiments, the 5′UTR comprises:

5′UTR-001 (Upstream UTR)
(SEQ ID NO. 327)
(GGGAAATAAGAGAGAAAAGAAGAGTAAGAAGAAATATAAGAGCCACC);
5′UTR-002 (Upstream UTR)
(SEQ ID NO. 328)
(GGGAGATCAGAGAGAAAAGAAGAGTAAGAAGAAATATAAGAGCCACC);
5′UTR-003 (Upstream UTR)
(SEQ ID NO. 329)
(GGAATAAAAGTCTCAACACAACATATACAAAACAAACGAATCTCAAGCA
ATCAAGCATTCTACTTCTATTGCAGCAATTTAAATCATTTCTTTTAAAGC
AAAAGCAATTTTCTGAAAATTTTCACCATTTACGAACGATAGCAAC);
5′UTR-004 (Upstream UTR)
(SEQ ID NO. 330)
(GGGAGACAAGCUUGGCAUUCCGGUACUGUUGGUAAAGCCACC);
5′UTR-005 (Upstream UTR)
(SEQ ID NO. 331)
(GGGAGATCAGAGAGAAAAGAAGAGTAAGAAGAAATATAAGAGCCACC);
UTR 5′UTR-006 (Upstream UTR)
(SEQ ID NO. 332)
(GGAATAAAAGTCTCAACACAACATATACAAAACAAACGAATCTCAAGCA
ATCAAGCATTCTACTTCTATTGCAGCAATTTAAATCATTTCTTTTAAAGC
AAAAGCAATTTTCTGAAAATTTTCACCATTTACGAACGATAGCAAC);
5′UTR-007 (Upstream UTR)
(SEQ ID NO. 333)
(GGGAGACAAGCUUGGCAUUCCGGUACUGUUGGUAAAGCCACC);
5′UTR-008 (Upstream UTR)
(SEQ ID NO. 334)
(GGGAATTAACAGAGAAAAGAAGAGTAAGAAGAAATATAAGAGCCACC);
5′UTR-009 (Upstream UTR)
(SEQ ID NO. 335)
(GGGAAATTAGACAGAAAAGAAGAGTAAGAAGAAATATAAGAGCCACC);
UTR 5′UTR-010, Upstream
(SEQ ID NO. 336)
(GGGAAATAAGAGAGTAAAGAACAGTAAGAAGAAATATAAGAGCCACC);
5′UTR-011 (Upstream UTR)
(SEQ ID NO. 337)
(GGGAAAAAAGAGAGAAAAGAAGACTAAGAAGAAATATAAGAGCCACC);
5′UTR-012 (Upstream UTR)
(SEQ ID NO. 338)
(GGGAAATAAGAGAGAAAAGAAGAGTAAGAAGATATATAAGAGCCACC);
5′UTR-013 (Upstream UTR)
(SEQ ID NO. 339)
(GGGAAATAAGAGACAAAACAAGAGTAAGAAGAAATATAAGAGCCACC);
5′UTR-014 (Upstream UTR)
(SEQ ID NO. 340)
(GGGAAATTAGAGAGTAAAGAACAGTAAGTAGAATTAAAAGAGCCACC);
5′UTR-15 (Upstream UTR)
(SEQ ID NO. 341)
(GGGAAATAAGAGAGAATAGAAGAGTAAGAAGAAATATAAGAGCCACC);
5′UTR-016 (Upstream UTR)
(SEQ ID NO. 342)
(GGGAAATAAGAGAGAAAAGAAGAGTAAGAAGAAAATTAAGAGCCACC);
5′UTR-017 (Upstream UTR)
(SEQ ID NO. 343)
(GGGAAATAAGAGAGAAAAGAAGAGTAAGAAGAAATTTAAGAGCCACC);
5′UTR-018 (Upstream UTR)
(SEQ ID NO. 344)
(TCAAGCTTTTGGACCCTCGTACAGAAGCTAATACGACTCACTATAGGGA
AATAAGAGAGAAAAGAAGAGTAAGAAGAAATATAAGAGCCACC);
142-3p 5′UTR-001 (Upstream UTR
including miR142-3p)
(SEQ ID NO. 345)
(TGATAATAGTCCATAAAGTAGGAAACACTACAGCTGGAGCCTCGGTGGC
CATGCTTCTTGCCCCTTGGGCCTCCCCCCAGCCCCTCCTCCCCTTCCTGC
ACCCGTACCCCCGTGGTCTTTGAATAAAGTCTGAGTGGGCGGC);
142-3p 5′UTR-002 (Upstream UTR
including miR142-3p)
(SEQ ID NO. 346)
(TGATAATAGGCTGGAGCCTCGGTGGCTCCATAAAGTAGGAAACACTACA
CATGCTTCTTGCCCCTTGGGCCTCCCCCCAGCCCCTCCTCCCCTTCCTGC
ACCCGTACCCCCGTGGTCTTTGAATAAAGTCTGAGTGGGCGGC);
142-3p 5′UTR-003 (Upstream UTR
including miR142-3p)
(SEQ ID NO. 347)
(TGATAATAGGCTGGAGCCTCGGTGGCCATGCTTCTTGCCCCTTCCATAA
AGTAGGAAACACTACATGGGCCTCCCCCCAGCCCCTCCTCCCCTTCCTGC
ACCCGTACCCCCGTGGTCTTTGAATAAAGTCTGAGTGGGCGGC);
142-3p 5′UTR-004 (Upstream UTR
including miR142-3p)
(SEQ ID NO. 348)
(TGATAATAGGCTGGAGCCTCGGTGGCCATGCTTCTTGCCCCTTGGGCCT
CCCCCCAGTCCATAAAGTAGGAAACACTACACCCCTCCTCCCCTTCCTGC
ACCCGTACCCCCGTGGTCTTTGAATAAAGTCTGAGTGGGCGGC);
142-3p 5′UTR-005 (Upstream UTR
including miR142-3p)
(SEQ ID NO. 349)
(TGATAATAGGCTGGAGCCTCGGTGGCCATGCTTCTTGCCCCTTGGGCCT
CCCCCCAGCCCCTCCTCCCCTTCTCCATAAAGTAGGAAACACTACACTGC
ACCCGTACCCCCGTGGTCTTTGAATAAAGTCTGAGTGGGCGGC);
142-3p 5′UTR-006 (Upstream UTR
including miR142-3p)
(SEQ ID NO. 350)
(TGATAATAGGCTGGAGCCTCGGTGGCCATGCTTCTTGCCCCTTGGGCCT
CCCCCCAGCCCCTCCTCCCCTTCCTGCACCCGTACCCCCTCCATAAAGTA
GGAAACACTACAGTGGTCTTTGAATAAAGTCTGAGTGGGCGGC);
or
142-3p 5′UTR-007 (Upstream UTR
including miR142-3p)
(SEQ ID NO. 351)
(TGATAATAGGCTGGAGCCTCGGTGGCCATGCTTCTTGCCCCTTGGGCCT
CCCCCCAGCCCCTCCTCCCCTTCCTGCACCCGTACCCCCGTGGTCTTTGA
ATAAAGTTCCATAAAGTAGGAAACACTACACTGAGTGGGCGGC).

In some embodiments, the 3′UTR comprises:

3′UTR-001 (Creatine Kinase UTR)
(SEQ ID NO. 352)
(GCGCCTGCCCACCTGCCACCGACTGCTGGAACCCAGCCAGTGGGAGGGCCTGGC
CCACCAGAGTCCTGCTCCCTCACTCCTCGCCCCGCCCCCTGTCCCAGAGTCCCAC
CTGGGGGCTCTCTCCACCCTTCTCAGAGTTCCAGTTTCAACCAGAGTTCCAACCA
ATGGGCTCCATCCTCTGGATTCTGGCCAATGAAATATCTCCCTGGCAGGGTCCTC
TTCTTTTCCCAGAGCTCCACCCCAACCAGGAGCTCTAGTTAATGGAGAGCTCCCA
GCACACTCGGAGCTTGTGCTTTGTCTCCACGCAAAGCGATAAATAAAAGCATTGG
TGGCCTTTGGTCTTTGAATAAAGCCTGAGTAGGAAGTCTAGA);
3′UTR-002 (Myoglobin UTR)
(SEQ ID NO. 353)
(GCCCCTGCCGCTCCCACCCCCACCCATCTGGGCCCCGGGTTCAAGAGAGAGCGG
GGTCTGATCTCGTGTAGCCATATAGAGTTTGCTTCTGAGTGTCTGCTTTGTTTAGT
AGAGGTGGGCAGGAGGAGCTGAGGGGCTGGGGCTGGGGTGTTGAAGTTGGCTTT
GCATGCCCAGCGATGCGCCTCCCTGTGGGATGTCATCACCCTGGGAACCGGGAG
TGGCCCTTGGCTCACTGTGTTCTGCATGGTTTGGATCTGAATTAATTGTCCTTTCT
TCTAAATCCCAACCGAACTTCTTCCAACCTCCAAACTGGCTGTAACCCCAAATCC
AAGCCATTAACTACACCTGACAGTAGCAATTGTCTGATTAATCACTGGCCCCTTG
AAGACAGCAGAATGTCCCTTTGCAATGAGGAGGAGATCTGGGCTGGGCGGGCCA
GCTGGGGAAGCATTTGACTATCTGGAACTTGTGTGTGCCTCCTCAGGTATGGCAG
TGACTCACCTGGTTTTAATAAAACAACCTGCAACATCTCATGGTCTTTGAATAAA
GCCTGAGTAGGAAGTCTAGA);
3′UTR-003 (α-actin UTR)
(SEQ ID NO. 354)
(ACACACTCCACCTCCAGCACGCGACTTCTCAGGACGACGAATCTTCTCAATGGG
GGGGCGGCTGAGCTCCAGCCACCCCGCAGTCACTTTCTTTGTAACAACTTCCGTT
GCTGCCATCGTAAACTGACACAGTGTTTATAACGTGTACATACATTAACTTATTA
CCTCATTTTGTTATTTTTCGAAACAAAGCCCTGTGGAAGAAAATGGAAAACTTGA
AGAAGCATTAAAGTCATTCTGTTAAGCTGCGTAAATGGTCTTTGAATAAAGCCTG
AGTAGGAAGTCTAGA);
3′UTR-004 (Albumin UTR)
(SEQ ID NO. 355)
(CATCACATTTAAAAGCATCTCAGCCTACCATGAGAATAAGAGAAAGAAAATGA
AGATCAAAAGCTTATTCATCTGTTTTTCTTTTTCGTTGGTGTAAAGCCAACACCCT
GTCTAAAAAACATAAATTTCTTTAATCATTTTGCCTCTTTTCTCTGTGCTTCAATT
AATAAAAAATGGAAAGAATCTAATAGAGTGGTACAGCACTGTTATTTTTCAAAG
ATGTGTTGCTATCCTGAAAATTCTGTAGGTTCTGTGGAAGTTCCAGTGTTCTCTCT
TATTCCACTTCGGTAGAGGATTTCTAGTTTCTTGTGGGCTAATTAAATAAATCATT
AATACTCTTCTAATGGTCTTTGAATAAAGCCTGAGTAGGAAGTCTAGA);
3′UTR-005 (α-globin UTR)
(SEQ ID NO. 356)
(GCTGCCTTCTGCGGGGCTTGCCTTCTGGCCATGCCCTTCTTCTCTCCCTTGCACCT
GTACCTCTTGGTCTTTGAATAAAGCCTGAGTAGGAAGGCGGCCGCTCGAGCATGC
ATCTAGA);
3′UTR-006 (G-CSF UTR)
(SEQ ID NO. 357)
(GCCAAGCCCTCCCCATCCCATGTATTTATCTCTATTTAATATTTATGTCTATTTAA
GCCTCATATTTAAAGACAGGGAAGAGCAGAACGGAGCCCCAGGCCTCTGTGTCC
TTCCCTGCATTTCTGAGTTTCATTCTCCTGCCTGTAGCAGTGAGAAAAAGCTCCTG
TCCTCCCATCCCCTGGACTGGGAGGTAGATAGGTAAATACCAAGTATTTATTACT
ATGACTGCTCCCCAGCCCTGGCTCTGCAATGGGCACTGGGATGAGCCGCTGTGAG
CCCCTGGTCCTGAGGGTCCCCACCTGGGACCCTTGAGAGTATCAGGTCTCCCACG
TGGGAGACAAGAAATCCCTGTTTAATATTTAAACAGCAGTGTTCCCCATCTGGGT
CCTTGCACCCCTCACTCTGGCCTCAGCCGACTGCACAGCGGCCCCTGCATCCCCT
TGGCTGTGAGGCCCCTGGACAAGCAGAGGTGGCCAGAGCTGGGAGGCATGGCCC
TGGGGTCCCACGAATTTGCTGGGGAATCTCGTTTTTCTTCTTAAGACTTTTGGGAC
ATGGTTTGACTCCCGAACATCACCGACGCGTCTCCTGTTTTTCTGGGTGGCCTCGG
GACACCTGCCCTGCCCCCACGAGGGTCAGGACTGTGACTCTTTTTAGGGCCAGGC
AGGTGCCTGGACATTTGCCTTGCTGGACGGGGACTGGGGATGTGGGAGGGAGCA
GACAGGAGGAATCATGTCAGGCCTGTGTGTGAAAGGAAGCTCCACTGTCACCCT
CCACCTCTTCACCCCCCACTCACCAGTGTCCCCTCCACTGTCACATTGTAACTGAA
CTTCAGGATAATAAAGTGTTTGCCTCCATGGTCTTTGAATAAAGCCTGAGTAGGA
AGGCGGCCGCTCGAGCATGCATCTAGA);
3′UTR-007 (Col1a2; collagen, type I, alpha 2 UTR)
(SEQ ID NO. 358)
(ACTCAATCTAAATTAAAAAAGAAAGAAATTTGAAAAAACTTTCTCTTTGCCATTT
CTTCTTCTTCTTTTTTAACTGAAAGCTGAATCCTTCCATTTCTTCTGCACATCTACT
TGCTTAAATTGTGGGCAAAAGAGAAAAAGAAGGATTGATCAGAGCATTGTGCAA
TACAGTTTCATTAACTCCTTCCCCCGCTCCCCCAAAAATTTGAATTTTTTTTTCAA
CACTCTTACACCTGTTATGGAAAATGTCAACCTTTGTAAGAAAACCAAAATAAAA
ATTGAAAAATAAAAACCATAAACATTTGCACCACTTGTGGCTTTTGAATATCTTC
CACAGAGGGAAGTTTAAAACCCAAACTTCCAAAGGTTTAAACTACCTCAAAACA
CTTTCCCATGAGTGTGATCCACATTGTTAGGTGCTGACCTAGACAGAGATGAACT
GAGGTCCTTGTTTTGTTTTGTTCATAATACAAAGGTGCTAATTAATAGTATTTCAG
ATACTTGAAGAATGTTGATGGTGCTAGAAGAATTTGAGAAGAAATACTCCTGTAT
TGAGTTGTATCGTGTGGTGTATTTTTTAAAAAATTTGATTTAGCATTCATATTTTC
CATCTTATTCCCAATTAAAAGTATGCAGATTATTTGCCCAAATCTTCTTCAGATTC
AGCATTTGTTCTTTGCCAGTCTCATTTTCATCTTCTTCCATGGTTCCACAGAAGCT
TTGTTTCTTGGGCAAGCAGAAAAATTAAATTGTACCTATTTTGTATATGTGAGAT
GTTTAAATAAATTGTGAAAAAAATGAAATAAAGCATGTTTGGTTTTCCAAAAGA
ACATAT);
3′UTR-008 (Col6a2; collagen, type VI, alpha 2 UTR)
(SEQ ID NO. 359)
(CGCCGCCGCCCGGGCCCCGCAGTCGAGGGTCGTGAGCCCACCCCGTCCATGGTG
CTAAGCGGGCCCGGGTCCCACACGGCCAGCACCGCTGCTCACTCGGACGACGCC
CTGGGCCTGCACCTCTCCAGCTCCTCCCACGGGGTCCCCGTAGCCCCGGCCCCCG
CCCAGCCCCAGGTCTCCCCAGGCCCTCCGCAGGCTGCCCGGCCTCCCTCCCCCTG
CAGCCATCCCAAGGCTCCTGACCTACCTGGCCCCTGAGCTCTGGAGCAAGCCCTG
ACCCAATAAAGGCTTTGAACCCAT);
3′UTR-009 (RPN1; ribophorin I UTR)
(SEQ ID NO. 360)
(GGGGCTAGAGCCCTCTCCGCACAGCGTGGAGACGGGGCAAGGAGGGGGGTTAT
TAGGATTGGTGGTTTTGTTTTGCTTTGTTTAAAGCCGTGGGAAAATGGCACAACT
TTACCTCTGTGGGAGATGCAACACTGAGAGCCAAGGGGTGGGAGTTGGGATAAT
TTTTATATAAAAGAAGTTTTTCCACTTTGAATTGCTAAAAGTGGCATTTTTCCTAT
GTGCAGTCACTCCTCTCATTTCTAAAATAGGGACGTGGCCAGGCACGGTGGCTCA
TGCCTGTAATCCCAGCACTTTGGGAGGCCGAGGCAGGCGGCTCACGAGGTCAGG
AGATCGAGACTATCCTGGCTAACACGGTAAAACCCTGTCTCTACTAAAAGTACAA
AAAATTAGCTGGGCGTGGTGGTGGGCACCTGTAGTCCCAGCTACTCGGGAGGCT
GAGGCAGGAGAAAGGCATGAATCCAAGAGGCAGAGCTTGCAGTGAGCTGAGAT
CACGCCATTGCACTCCAGCCTGGGCAACAGTGTTAAGACTCTGTCTCAAATATAA
ATAAATAAATAAATAAATAAATAAATAAATAAAAATAAAGCGAGATGTTGCCCT
CAAA);
3′UTR-010 (LRP1; low density lipoprotein receptor-related
protein 1 UTR)
(SEQ ID NO. 361)
(GGCCCTGCCCCGTCGGACTGCCCCCAGAAAGCCTCCTGCCCCCTGCCAGTGAAG
TCCTTCAGTGAGCCCCTCCCCAGCCAGCCCTTCCCTGGCCCCGCCGGATGTATAA
ATGTAAAAATGAAGGAATTACATTTTATATGTGAGCGAGCAAGCCGGCAAGCGA
GCACAGTATTATTTCTCCATCCCCTCCCTGCCTGCTCCTTGGCACCCCCATGCTGC
CTTCAGGGAGACAGGCAGGGAGGGCTTGGGGCTGCACCTCCTACCCTCCCACCA
GAACGCACCCCACTGGGAGAGCTGGTGGTGCAGCCTTCCCCTCCCTGTATAAGAC
ACTTTGCCAAGGCTCTCCCCTCTCGCCCCATCCCTGCTTGCCCGCTCCCACAGCTT
CCTGAGGGCTAATTCTGGGAAGGGAGAGTTCTTTGCTGCCCCTGTCTGGAAGACG
TGGCTCTGGGTGAGGTAGGCGGGAAAGGATGGAGTGTTTTAGTTCTTGGGGGAG
GCCACCCCAAACCCCAGCCCCAACTCCAGGGGCACCTATGAGATGGCCATGCTC
AACCCCCCTCCCAGACAGGCCCTCCCTGTCTCCAGGGCCCCCACCGAGGTTCCCA
GGGCTGGAGACTTCCTCTGGTAAACATTCCTCCAGCCTCCCCTCCCCTGGGGACG
CCAAGGAGGTGGGCCACACCCAGGAAGGGAAAGCGGGCAGCCCCGTTTTGGGG
ACGTGAACGTTTTAATAATTTTTGCTGAATTCCTTTACAACTAAATAACACAGAT
ATTGTTATAAATAAAATTGT);
3′UTR-011 (Nnt1; cardiotrophin-like cytokine factor 1 UTR)
(SEQ ID NO. 362)
(ATATTAAGGATCAAGCTGTTAGCTAATAATGCCACCTCTGCAGTTTTGGGAACA
GGCAAATAAAGTATCAGTATACATGGTGATGTACATCTGTAGCAAAGCTCTTGGA
GAAAATGAAGACTGAAGAAAGCAAAGCAAAAACTGTATAGAGAGATTTTTCAAA
AGCAGTAATCCCTCAATTTTAAAAAAGGATTGAAAATTCTAAATGTCTTTCTGTG
CATATTTTTTGTGTTAGGAATCAAAAGTATTTTATAAAAGGAGAAAGAACAGCCT
CATTTTAGATGTAGTCCTGTTGGATTTTTTATGCCTCCTCAGTAACCAGAAATGTT
TTAAAAAACTAAGTGTTTAGGATTTCAAGACAACATTATACATGGCTCTGAAATA
TCTGACACAATGTAAACATTGCAGGCACCTGCATTTTATGTTTTTTTTTTCAACAA
ATGTGACTAATTTGAAACTTTTATGAACTTCTGAGCTGTCCCCTTGCAATTCAACC
GCAGTTTGAATTAATCATATCAAATCAGTTTTAATTTTTTAAATTGTACTTCAGAG
TCTATATTTCAAGGGCACATTTTCTCACTACTATTTTAATACATTAAAGGACTAAA
TAATCTTTCAGAGATGCTGGAAACAAATCATTTGCTTTATATGTTTCATTAGAATA
CCAATGAAACATACAACTTGAAAATTAGTAATAGTATTTTTGAAGATCCCATTTC
TAATTGGAGATCTCTTTAATTTCGATCAACTTATAATGTGTAGTACTATATTAAGT
GCACTTGAGTGGAATTCAACATTTGACTAATAAAATGAGTTCATCATGTTGGCAA
GTGATGTGGCAATTATCTCTGGTGACAAAAGAGTAAAATCAAATATTTCTGCCTG
TTACAAATATCAAGGAAGACCTGCTACTATGAAATAGATGACATTAATCTGTCTT
CACTGTTTATAATACGGATGGATTTTTTTTCAAATCAGTGTGTGTTTTGAGGTCTT
ATGTAATTGATGACATTTGAGAGAAATGGTGGCTTTTTTTAGCTACCTCTTTGTTC
ATTTAAGCACCAGTAAAGATCATGTCTTTTTATAGAAGTGTAGATTTTCTTTGTGA
CTTTGCTATCGTGCCTAAAGCTCTAAATATAGGTGAATGTGTGATGAATACTCAG
ATTATTTGTCTCTCTATATAATTAGTTTGGTACTAAGTTTCTCAAAAAATTATTAA
CACATGAAAGACAATCTCTAAACCAGAAAAAGAAGTAGTACAAATTTTGTTACT
GTAATGCTCGCGTTTAGTGAGTTTAAAACACACAGTATCTTTTGGTTTTATAATCA
GTTTCTATTTTGCTGTGCCTGAGATTAAGATCTGTGTATGTGTGTGTGTGTGTGTG
TGCGTTTGTGTGTTAAAGCAGAAAAGACTTTTTTAAAAGTTTTAAGTGATAAATG
CAATTTGTTAATTGATCTTAGATCACTAGTAAACTCAGGGCTGAATTATACCATG
TATATTCTATTAGAAGAAAGTAAACACCATCTTTATTCCTGCCCTTTTTCTTCTCT
CAAAGTAGTTGTAGTTATATCTAGAAAGAAGCAATTTTGATTTCTTGAAAAGGTA
GTTCCTGCACTCAGTTTAAACTAAAAATAATCATACTTGGATTTTATTTATTTTTG
TCATAGTAAAAATTTTAATTTATATATATTTTTATTTAGTATTATCTTATTCTTTGC
TATTTGCCAATCCTTTGTCATCAATTGTGTTAAATGAATTGAAAATTCATGCCCTG
TTCATTTTATTTTACTTTATTGGTTAGGATATTTAAAGGATTTTTGTATATATAATT
TCTTAAATTAATATTCCAAAAGGTTAGTGGACTTAGATTATAAATTATGGCAAAA
ATCTAAAAACAACAAAAATGATTTTTATACATTCTATTTCATTATTCCTCTTTTTC
CAATAAGTCATACAATTGGTAGATATGACTTATTTTATTTTTGTATTATTCACTAT
ATCTTTATGATATTTAAGTATAAATAATTAAAAAAATTTATTGTACCTTATAGTCT
GTCACCAAAAAAAAAAAATTATCTGTAGGTAGTGAAATGCTAATGTTGATTTGTC
TTTAAGGGCTTGTTAACTATCCTTTATTTTCTCATTTGTCTTAAATTAGGAGTTTGT
GTTTAAATTACTCATCTAAGCAAAAAATGTATATAAATCCCATTACTGGGTATAT
ACCCAAAGGATTATAAATCATGCTGCTATAAAGACACATGCACACGTATGTTTAT
TGCAGCACTATTCACAATAGCAAAGACTTGGAACCAACCCAAATGTCCATCAAT
GATAGACTTGATTAAGAAAATGTGCACATATACACCATGGAATACTATGCAGCC
ATAAAAAAGGATGAGTTCATGTCCTTTGTAGGGACATGGATAAAGCTGGAAACC
ATCATTCTGAGCAAACTATTGCAAGGACAGAAAACCAAACACTGCATGTTCTCAC
TCATAGGTGGGAATTGAACAATGAGAACACTTGGACACAAGGTGGGGAACACCA
CACACCAGGGCCTGTCATGGGGTGGGGGGAGTGGGGAGGGATAGCATTAGGAG
ATATACCTAATGTAAATGATGAGTTAATGGGTGCAGCACACCAACATGGCACAT
GTATACATATGTAGCAAACCTGCACGTTGTGCACATGTACCCTAGAACTTAAAGT
ATAATTAAAAAAAAAAAGAAAACAGAAGCTATTTATAAAGAAGTTATTTGCTGA
AATAAATGTGATCTTTCCCATTAAAAAAATAAAGAAATTTTGGGGTAAAAAAAC
ACAATATATTGTATTCTTGAAAAATTCTAAGAGAGTGGATGTGAAGTGTTCTCAC
CACAAAAGTGATAACTAATTGAGGTAATGCACATATTAATTAGAAAGATTTTGTC
ATTCCACAATGTATATATACTTAAAAATATGTTATACACAATAAATACATACATT
AAAAAATAAGTAAATGTA);
3′UTR-012 (Col6a1; collagen, type VI, alpha 1 UTR)
(SEQ ID NO. 363)
(CCCACCCTGCACGCCGGCACCAAACCCTGTCCTCCCACCCCTCCCCACTCATCAC
TAAACAGAGTAAAATGTGATGCGAATTTTCCCGACCAACCTGATTCGCTAGATTT
TTTTTAAGGAAAAGCTTGGAAAGCCAGGACACAACGCTGCTGCCTGCTTTGTGCA
GGGTCCTCCGGGGCTCAGCCCTGAGTTGGCATCACCTGCGCAGGGCCCTCTGGGG
CTCAGCCCTGAGCTAGTGTCACCTGCACAGGGCCCTCTGAGGCTCAGCCCTGAGC
TGGCGTCACCTGTGCAGGGCCCTCTGGGGCTCAGCCCTGAGCTGGCCTCACCTGG
GTTCCCCACCCCGGGCTCTCCTGCCCTGCCCTCCTGCCCGCCCTCCCTCCTGCCTG
CGCAGCTCCTTCCCTAGGCACCTCTGTGCTGCATCCCACCAGCCTGAGCAAGACG
CCCTCTCGGGGCCTGTGCCGCACTAGCCTCCCTCTCCTCTGTCCCCATAGCTGGTT
TTTCCCACCAATCCTCACCTAACAGTTACTTTACAATTAAACTCAAAGCAAGCTC
TTCTCCTCAGCTTGGGGCAGCCATTGGCCTCTGTCTCGTTTTGGGAAACCAAGGT
CAGGAGGCCGTTGCAGACATAAATCTCGGCGACTCGGCCCCGTCTCCTGAGGGT
CCTGCTGGTGACCGGCCTGGACCTTGGCCCTACAGCCCTGGAGGCCGCTGCTGAC
CAGCACTGACCCCGACCTCAGAGAGTACTCGCAGGGGCGCTGGCTGCACTCAAG
ACCCTCGAGATTAACGGTGCTAACCCCGTCTGCTCCTCCCTCCCGCAGAGACTGG
GGCCTGGACTGGACATGAGAGCCCCTTGGTGCCACAGAGGGCTGTGTCTTACTAG
AAACAACGCAAACCTCTCCTTCCTCAGAATAGTGATGTGTTCGACGTTTTATCAA
AGGCCCCCTTTCTATGTTCATGTTAGTTTTGCTCCTTCTGTGTTTTTTTCTGAACCA
TATCCATGTTGCTGACTTTTCCAAATAAAGGTTTTCACTCCTCTC);
3′UTR-013 (Calr; calreticulin UTR)
(SEQ ID NO. 364)
(AGAGGCCTGCCTCCAGGGCTGGACTGAGGCCTGAGCGCTCCTGCCGCAGAGCTG
GCCGCGCCAAATAATGTCTCTGTGAGACTCGAGAACTTTCATTTTTTTCCAGGCT
GGTTCGGATTTGGGGTGGATTTTGGTTTTGTTCCCCTCCTCCACTCTCCCCCACCC
CCTCCCCGCCCTTTTTTTTTTTTTTTTTTAAACTGGTATTTTATCTTTGATTCTCCTT
CAGCCCTCACCCCTGGTTCTCATCTTTCTTGATCAACATCTTTTCTTGCCTCTGTCC
CCTTCTCTCATCTCTTAGCTCCCCTCCAACCTGGGGGGCAGTGGTGTGGAGAAGC
CACAGGCCTGAGATTTCATCTGCTCTCCTTCCTGGAGCCCAGAGGAGGGCAGCAG
AAGGGGGTGGTGTCTCCAACCCCCCAGCACTGAGGAAGAACGGGGCTCTTCTCA
TTTCACCCCTCCCTTTCTCCCCTGCCCCCAGGACTGGGCCACTTCTGGGTGGGGCA
GTGGGTCCCAGATTGGCTCACACTGAGAATGTAAGAACTACAAACAAAATTTCT
ATTAAATTAAATTTTGTGTCTCC);
3′UTR-014 (Col1al; collagen, type I, alpha 1 UTR)
(SEQ ID NO. 365)
(CTCCCTCCATCCCAACCTGGCTCCCTCCCACCCAACCAACTTTCCCCCCAACCCG
GAAACAGACAAGCAACCCAAACTGAACCCCCTCAAAAGCCAAAAAATGGGAGA
CAATTTCACATGGACTTTGGAAAATATTTTTTTCCTTTGCATTCATCTCTCAAACT
TAGTTTTTATCTTTGACCAACCGAACATGACCAAAAACCAAAAGTGCATTCAACC
TTACCAAAAAAAAAAAAAAAAAAAGAATAAATAAATAACTTTTTAAAAAAGGA
AGCTTGGTCCACTTGCTTGAAGACCCATGCGGGGGTAAGTCCCTTTCTGCCCGTT
GGGCTTATGAAACCCCAATGCTGCCCTTTCTGCTCCTTTCTCCACACCCCCCTTGG
GGCCTCCCCTCCACTCCTTCCCAAATCTGTCTCCCCAGAAGACACAGGAAACAAT
GTATTGTCTGCCCAGCAATCAAAGGCAATGCTCAAACACCCAAGTGGCCCCCAC
CCTCAGCCCGCTCCTGCCCGCCCAGCACCCCCAGGCCCTGGGGGACCTGGGGTTC
TCAGACTGCCAAAGAAGCCTTGCCATCTGGCGCTCCCATGGCTCTTGCAACATCT
CCCCTTCGTTTTTGAGGGGGTCATGCCGGGGGAGCCACCAGCCCCTCACTGGGTT
CGGAGGAGAGTCAGGAAGGGCCACGACAAAGCAGAAACATCGGATTTGGGGAA
CGCGTGTCAATCCCTTGTGCCGCAGGGCTGGGCGGGAGAGACTGTTCTGTTCCTT
GTGTAACTGTGTTGCTGAAAGACTACCTCGTTCTTGTCTTGATGTGTCACCGGGG
CAACTGCCTGGGGGCGGGGATGGGGGCAGGGTGGAAGCGGCTCCCCATTTTATA
CCAAAGGTGCTACATCTATGTGATGGGTGGGGTGGGGAGGGAATCACTGGTGCT
ATAGAAATTGAGATGCCCCCCCAGGCCAGCAAATGTTCCTTTTTGTTCAAAGTCT
ATTTTTATTCCTTGATATTTTTCTTTTTTTTTTTTTTTTTTTGTGGATGGGGACTTGT
GAATTTTTCTAAAGGTGCTATTTAACATGGGAGGAGAGCGTGTGCGGCTCCAGCC
CAGCCCGCTGCTCACTTTCCACCCTCTCTCCACCTGCCTCTGGCTTCTCAGGCCTC
TGCTCTCCGACCTCTCTCCTCTGAAACCCTCCTCCACAGCTGCAGCCCATCCTCCC
GGCTCCCTCCTAGTCTGTCCTGCGTCCTCTGTCCCCGGGTTTCAGAGACAACTTCC
CAAAGCACAAAGCAGTTTTTCCCCCTAGGGGTGGGAGGAAGCAAAAGACTCTGT
ACCTATTTTGTATGTGTATAATAATTTGAGATGTTTTTAATTATTTTGATTGCTGG
AATAAAGCATGTGGAAATGACCCAAACATAATCCGCAGTGGCCTCCTAATTTCCT
TCTTTGGAGTTGGGGGAGGGGTAGACATGGGGAAGGGGCTTTGGGGTGATGGGC
TTGCCTTCCATTCCTGCCCTTTCCCTCCCCACTATTCTCTTCTAGATCCCTCCATAA
CCCCACTCCCCTTTCTCTCACCCTTCTTATACCGCAAACCTTTCTACTTCCTCTTTC
ATTTTCTATTCTTGCAATTTCCTTGCACCTTTTCCAAATCCTCTTCTCCCCTGCAAT
ACCATACAGGCAATCCACGTGCACAACACACACACACACTCTTCACATCTGGGG
TTGTCCAAACCTCATACCCACTCCCCTTCAAGCCCATCCACTCTCCACCCCCTGGA
TGCCCTGCACTTGGTGGCGGTGGGATGCTCATGGATACTGGGAGGGTGAGGGGA
GTGGAACCCGTGAGGAGGACCTGGGGGCCTCTCCTTGAACTGACATGAAGGGTC
ATCTGGCCTCTGCTCCCTTCTCACCCACGCTGACCTCCTGCCGAAGGAGCAACGC
AACAGGAGAGGGGTCTGCTGAGCCTGGCGAGGGTCTGGGAGGGACCAGGAGGA
AGGCGTGCTCCCTGCTCGCTGTCCTGGCCCTGGGGGAGTGAGGGAGACAGACAC
CTGGGAGAGCTGTGGGGAAGGCACTCGCACCGTGCTCTTGGGAAGGAAGGAGAC
CTGGCCCTGCTCACCACGGACTGGGTGCCTCGACCTCCTGAATCCCCAGAACACA
ACCCCCCTGGGCTGGGGTGGTCTGGGGAACCATCGTGCCCCCGCCTCCCGCCTAC
TCCTTTTTAAGCTT);
3′UTR-015 (Plod 1; procollagen-lysine, 2-oxoglutarate
5-dioxygenase 1 UTR)
(SEQ ID NO. 366)
(TTGGCCAGGCCTGACCCTCTTGGACCTTTCTTCTTTGCCGACAACCACTGCCCAG
CAGCCTCTGGGACCTCGGGGTCCCAGGGAACCCAGTCCAGCCTCCTGGCTGTTGA
CTTCCCATTGCTCTTGGAGCCACCAATCAAAGAGATTCAAAGAGATTCCTGCAGG
CCAGAGGCGGAACACACCTTTATGGCTGGGGCTCTCCGTGGTGTTCTGGACCCAG
CCCCTGGAGACACCATTCACTTTTACTGCTTTGTAGTGACTCGTGCTCTCCAACCT
GTCTTCCTGAAAAACCAAGGCCCCCTTCCCCCACCTCTTCCATGGGGTGAGACTT
GAGCAGAACAGGGGCTTCCCCAAGTTGCCCAGAAAGACTGTCTGGGTGAGAAGC
CATGGCCAGAGCTTCTCCCAGGCACAGGTGTTGCACCAGGGACTTCTGCTTCAAG
TTTTGGGGTAAAGACACCTGGATCAGACTCCAAGGGCTGCCCTGAGTCTGGGACT
TCTGCCTCCATGGCTGGTCATGAGAGCAAACCGTAGTCCCCTGGAGACAGCGACT
CCAGAGAACCTCTTGGGAGACAGAAGAGGCATCTGTGCACAGCTCGATCTTCTA
CTTGCCTGTGGGGAGGGGAGTGACAGGTCCACACACCACACTGGGTCACCCTGT
CCTGGATGCCTCTGAAGAGAGGGACAGACCGTCAGAAACTGGAGAGTTTCTATT
AAAGGTCATTTAAACCA);
3′UTR-016 (Nucb 1; nucleobindin 1 UTR)
(SEQ ID NO. 367)
(TCCTCCGGGACCCCAGCCCTCAGGATTCCTGATGCTCCAAGGCGACTGATGGGC
GCTGGATGAAGTGGCACAGTCAGCTTCCCTGGGGGCTGGTGTCATGTTGGGCTCC
TGGGGCGGGGGCACGGCCTGGCATTTCACGCATTGCTGCCACCCCAGGTCCACCT
GTCTCCACTTTCACAGCCTCCAAGTCTGTGGCTCTTCCCTTCTGTCCTCCGAGGGG
CTTGCCTTCTCTCGTGTCCAGTGAGGTGCTCAGTGATCGGCTTAACTTAGAGAAG
CCCGCCCCCTCCCCTTCTCCGTCTGTCCCAAGAGGGTCTGCTCTGAGCCTGCGTTC
CTAGGTGGCTCGGCCTCAGCTGCCTGGGTTGTGGCCGCCCTAGCATCCTGTATGC
CCACAGCTACTGGAATCCCCGCTGCTGCTCCGGGCCAAGCTTCTGGTTGATTAAT
GAGGGCATGGGGTGGTCCCTCAAGACCTTCCCCTACCTTTTGTGGAACCAGTGAT
GCCTCAAAGACAGTGTCCCCTCCACAGCTGGGTGCCAGGGGCAGGGGATCCTCA
GTATAGCCGGTGAACCCTGATACCAGGAGCCTGGGCCTCCCTGAACCCCTGGCTT
CCAGCCATCTCATCGCCAGCCTCCTCCTGGACCTCTTGGCCCCCAGCCCCTTCCCC
ACACAGCCCCAGAAGGGTCCCAGAGCTGACCCCACTCCAGGACCTAGGCCCAGC
CCCTCAGCCTCATCTGGAGCCCCTGAAGACCAGTCCCACCCACCTTTCTGGCCTC
ATCTGACACTGCTCCGCATCCTGCTGTGTGTCCTGTTCCATGTTCCGGTTCCATCC
AAATACACTTTCTGGAACAAA);
3′UTR-017 (α-globin)
(SEQ ID NO. 368)
(GCTGGAGCCTCGGTGGCCATGCTTCTTGCCCCTTGGGCCTCCCCCCAGCCCCTCC
TCCCCTTCCTGCACCCGTACCCCCGTGGTCTTTGAATAAAGTCTGAGTGGGCGGC);
or
3′UTR-018
(SEQ ID NO. 369)
(TGATAATAGGCTGGAGCCTCGGTGGCCATGCTTCTTGCCCCTTGGGCCTCCCCCC
AGCCCCTCCTCCCCTTCCTGCACCCGTACCCCCGTGGTCTTTGAATAAAGTCTGA
GTGGGCGGC).

In certain embodiments, the 5′UTR and/or 3′UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′UTR sequences comprising any of SEQ ID NOs: 327-351 and/or 3′UTR sequences comprises any of SEQ ID NOs: 352-369, and any combination thereof.

The polynucleotides of the invention can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

It is also within the scope of the present invention to have patterned UTRs. As used herein “patterned UTRs” include a repeating or alternating pattern, such as ABABAB or AABBAABBAABB or ABCABCABC or variants thereof repeated once, twice, or more than 3 times. In these patterns, each letter, A, B, or C represent a different UTR nucleic acid sequence.

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide of the invention comprises 5′ and/or 3′ sequence associated with the 5′ and/or 3′ ends of rubella virus (RV) genomic RNA, respectively, or deletion derivatives thereof, including the 5′ proximal open reading frame of RV RNA encoding nonstructural proteins (e.g., see Pogue et al., J. Virol. 67(12):7106-7117, the contents of which are incorporated herein by reference in their entirety). Viral capsid sequences can also be used as a translational enhancer, e.g., the 5′ portion of a capsid sequence, (e.g., semliki forest virus and sindbis virus capsid RNAs as described in Sjöberg et al., Biotechnology (NY) 1994 12(11):1127-1131, and Frolov and Schlesinger J. Virol. 1996 70(2):1182-1190, the contents of each of which are incorporated herein by reference in their entirety). In some embodiments, the polynucleotide comprises an IRES instead of a 5′UTR sequence. In some embodiments, the polynucleotide comprises an ORF and a viral capsid sequence. In some embodiments, the polynucleotide comprises a synthetic 5′UTR in combination with a non-synthetic 3′UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can include those described in US2009/0226470, incorporated herein by reference in its entirety, and others known in the art. As a non-limiting example, the TEE can be located between the transcription promoter and the start codon. In some embodiments, the 5′UTR comprises a TEE.

In one aspect, a TEE is a conserved element in a UTR that can promote translational activity of a nucleic acid such as, but not limited to, cap-dependent or cap-independent translation. The conservation of these sequences has been shown across 14 species including humans. See, e.g., Panek et al., “An evolutionary conserved pattern of 18S rRNA sequence complementarity to mRNA 5′UTRs and its implications for eukaryotic gene translation regulation,” Nucleic Acids Research 2013, doi:10.1093/nar/gkt548, incorporated herein by reference in its entirety.

In one non-limiting example, the TEE comprises the TEE sequence in the 5′-leader of the Gtx homeodomain protein. See Chappell et al., PNAS 2004 101:9590-9594, incorporated herein by reference in its entirety.

In another non-limiting example, the TEE comprises a TEE having one or more of the sequences of SEQ ID NOs: 1-35 in US2009/0226470, US2013/0177581, and WO2009/075886; SEQ ID NOs: 1-5 and 7-645 in WO2012/009644; and SEQ ID NO: 1 WO1999/024595, U.S. Pat. Nos. 6,310,197, and 6,849,405; the contents of each of which are incorporated herein by reference in their entirety.

In some embodiments, the TEE is an internal ribosome entry site (IRES), HCV-IRES, or an IRES element such as, but not limited to, those described in: U.S. Pat. No. 7,468,275, US2007/0048776, US2011/0124100, WO2007/025008, and WO2001/055369; the contents of each of which re incorporated herein by reference in their entirety. The IRES elements can include, but are not limited to, the Gtx sequences (e.g., Gtx9-nt, Gtx8-nt, Gtx7-nt) as described by Chappell et al., PNAS 2004 101:9590-9594, Zhou et al., PNAS 2005 102:6273-6278, US2007/0048776, US2011/0124100, and WO2007/025008; the contents of each of which are incorporated herein by reference in their entirety.

“Translational enhancer polynucleotide” or “translation enhancer polynucleotide sequence” refer to a polynucleotide that includes one or more of the TEE provided herein and/or known in the art (see. e.g., U.S. Pat. Nos. 6,310,197, 6,849,405, 7,456,273, 7,183,395, US2009/0226470, US2007/0048776, US2011/0124100, US2009/0093049, US2013/0177581, WO2009/075886, WO2007/025008, WO2012/009644, WO2001/055371, WO1999/024595, EP2610341A1, and EP2610340A1; the contents of each of which are incorporated herein by reference in their entirety), or their variants, homologs, or functional derivatives. In some embodiments, the polynucleotide of the invention comprises one or multiple copies of a TEE.

The TEE in a translational enhancer polynucleotide can be organized in one or more sequence segments. A sequence segment can harbor one or more of the TEEs provided herein, with each TEE being present in one or more copies. When multiple sequence segments are present in a translational enhancer polynucleotide, they can be homogenous or heterogeneous. Thus, the multiple sequence segments in a translational enhancer polynucleotide can harbor identical or different types of the TEE provided herein, identical or different number of copies of each of the TEE, and/or identical or different organization of the TEE within each sequence segment. In one embodiment, the polynucleotide of the invention comprises a translational enhancer polynucleotide sequence.

In some embodiments, a 5′UTR and/or 3′UTR of a polynucleotide of the invention comprises at least one TEE or portion thereof that is disclosed in: WO1999/024595, WO2012/009644, WO2009/075886, WO2007/025008, WO1999/024595, WO2001/055371, EP2610341A1, EP2610340A1, U.S. Pat. Nos. 6,310,197, 6,849,405, 7,456,273, 7,183,395, US2009/0226470, US2011/0124100, US2007/0048776, US2009/0093049, or US2013/0177581, the contents of each are incorporated herein by reference in their entirety. In some embodiments, a 5′UTR and/or 3′UTR of a polynucleotide of the invention comprises a TEE that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to a TEE disclosed in: US2009/0226470, US2007/0048776, US2013/0177581, US2011/0124100, WO1999/024595, WO2012/009644, WO2009/075886, WO2007/025008, EP2610341A1, EP2610340A1, U.S. Pat. Nos. 6,310,197, 6,849,405, 7,456,273, 7,183,395, Chappell et al., PNAS 2004 101:9590-9594, Zhou et al., PNAS 2005 102:6273-6278, and Supplemental Table 1 and in Supplemental Table 2 of Wellensiek et al., “Genome-wide profiling of human cap-independent translation-enhancing elements,” Nature Methods 2013, DOI:10.1038/NMETH.2522; the contents of each of which are incorporated herein by reference in their entirety.

In some embodiments, a 5′UTR and/or 3′UTR of a polynucleotide of the invention comprises a TEE which is selected from a 5-30 nucleotide fragment, a 5-25 nucleotide fragment, a 5-20 nucleotide fragment, a 5-15 nucleotide fragment, or a 5-10 nucleotide fragment (including a fragment of 5, 6, 7, 8, 9, 10, 11, 12, 13, 14, 15, 16, 17, 18, 19, 20, 21, 22, 23, 24, 25, 26, 27, 28, 29, or 30 nucleotides) of a TEE sequence disclosed in: US2009/0226470, US2007/0048776, US2013/0177581, US2011/0124100, WO1999/024595, WO2012/009644, WO2009/075886, WO2007/025008, EP2610341A1, EP2610340A1, U.S. Pat. Nos. 6,310,197, 6,849,405, 7,456,273, 7,183,395, Chappell et al., PNAS 2004 101:9590-9594, Zhou et al., PNAS 2005 102:6273-6278, and Supplemental Table 1 and in Supplemental Table 2 of Wellensiek et al., “Genome-wide profiling of human cap-independent translation-enhancing elements,” Nature Methods 2013, DOI:10.1038/NMETH.2522.

In some embodiments, a 5′UTR and/or 3′UTR of a polynucleotide of the invention comprises a TEE which is a transcription regulatory element described in any of U.S. Pat. Nos. 7,456,273, 7,183,395, US2009/0093049, and WO2001/055371, the contents of each of which are incorporated herein by reference in their entirety. The transcription regulatory elements can be identified by methods known in the art, such as, but not limited to, the methods described in U.S. Pat. Nos. 7,456,273, 7,183,395, US2009/0093049, and WO2001/055371.

In some embodiments, a 5′UTR and/or 3′UTR comprising at least one TEE described herein can be incorporated in a monocistronic sequence such as, but not limited to, a vector system or a nucleic acid vector. As non-limiting examples, the vector systems and nucleic acid vectors can include those described in U.S. Pat. Nos. 7,456,273, 7,183,395, US2007/0048776, US2009/0093049, US2011/0124100, WO2007/025008, and WO2001/055371.

In some embodiments, a 5′UTR and/or 3′UTR of a polynucleotide of the invention comprises a TEE or portion thereof described herein. In some embodiments, the TEEs in the 3′UTR can be the same and/or different from the TEE located in the 5′UTR.

In some embodiments, a 5′UTR and/or 3′UTR of a polynucleotide of the invention can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In one embodiment, the 5′UTR of a polynucleotide of the invention can include 1-60, 1-55, 1-50, 1-45, 1-40, 1-35, 1-30, 1-25, 1-20, 1-15, 1-10, 9, 8, 7, 6, 5, 4, 3, 2, or 1 TEE sequences. The TEE sequences in the 5′UTR of the polynucleotide of the invention can be the same or different TEE sequences. A combination of different TEE sequences in the 5′UTR of the polynucleotide of the invention can include combinations in which more than one copy of any of the different TEE sequences are incorporated. The TEE sequences can be in a pattern such as ABABAB or AABBAABBAABB or ABCABCABC or variants thereof repeated one, two, three, or more than three times. In these patterns, each letter, A, B, or C represent a different TEE nucleotide sequence.

In some embodiments, the TEE can be identified by the methods described in US2007/0048776, US2011/0124100, WO2007/025008, WO2012/009644, the contents of each of which are incorporated herein by reference in their entirety.

In some embodiments, the 5′UTR and/or 3′UTR comprises a spacer to separate two TEE sequences. As a non-limiting example, the spacer can be a 15 nucleotide spacer and/or other spacers known in the art. As another non-limiting example, the 5′UTR and/or 3′UTR comprises a TEE sequence-spacer module repeated at least once, at least twice, at least 3 times, at least 4 times, at least 5 times, at least 6 times, at least 7 times, at least 8 times, at least 9 times, at least 10 times, or more than 10 times in the 5′UTR and/or 3′UTR, respectively. In some embodiments, the 5′UTR and/or 3′UTR comprises a TEE sequence-spacer module repeated 1, 2, 3, 4, 5, 6, 7, 8, 9, or 10 times.

In some embodiments, the spacer separating two TEE sequences can include other sequences known in the art that can regulate the translation of the polynucleotide of the invention, e.g., miR sequences described herein (e.g., miR binding sites and miR seeds). As a non-limiting example, each spacer used to separate two TEE sequences can include a different miR sequence or component of a miR sequence (e.g., miR seed sequence).

In some embodiments, a polynucleotide of the invention comprises a miR and/or TEE sequence. In some embodiments, the incorporation of a miR sequence and/or a TEE sequence into a polynucleotide of the invention can change the shape of the stem loop region, which can increase and/or decrease translation. See e.g., Kedde et al., Nature Cell Biology 2010 12(10):1014-20, herein incorporated by reference in its entirety).

MicroRNA (miRNA) Binding Sites

Polynucleotides of the invention can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, polynucleotides including such regulatory elements are referred to as including “sensor sequences”. Non-limiting examples of sensor sequences are described in U.S. Publication 2014/0200261, the contents of which are incorporated herein by reference in their entirety.

In some embodiments, a polynucleotide (e.g., a ribonucleic acid (RNA), e.g., a messenger RNA (mRNA)) of the invention comprises an open reading frame (ORF) encoding a polypeptide of interest and further comprises one or more miRNA binding site(s). Inclusion or incorporation of miRNA binding site(s) provides for regulation of polynucleotides of the invention, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a polynucleotide and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a polynucleotide (e.g., a ribonucleic acid (RNA), e.g., a messenger RNA (mRNA)) of the invention comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a polynucleotide, e.g., within a DNA or within an RNA transcript, including in the 5′UTR and/or 3′UTR, that has sufficient complementarity to all or a region of a miRNA to interact with, associate with or bind to the miRNA. In some embodiments, a polynucleotide of the invention comprising an ORF encoding a polypeptide of interest and further comprises one or more miRNA binding site(s). In exemplary embodiments, a 5′UTR and/or 3′UTR of the polynucleotide (e.g., a ribonucleic acid (RNA), e.g., a messenger RNA (mRNA)) comprises the one or more miRNA binding site(s).

A miRNA binding site having sufficient complementarity to a miRNA refers to a degree of complementarity sufficient to facilitate miRNA-mediated regulation of a polynucleotide, e.g., miRNA-mediated translational repression or degradation of the polynucleotide. In exemplary aspects of the invention, a miRNA binding site having sufficient complementarity to the miRNA refers to a degree of complementarity sufficient to facilitate miRNA-mediated degradation of the polynucleotide, e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with an miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the polynucleotide comprising the miRNA binding site. In other embodiments, the miRNA binding site has imperfect complementarity so that a RISC complex comprising the miRNA induces instability in the polynucleotide comprising the miRNA binding site. In another embodiment, the miRNA binding site has imperfect complementarity so that a RISC complex comprising the miRNA represses transcription of the polynucleotide comprising the miRNA binding site.

In some embodiments, the miRNA binding site has one, two, three, four, five, six, seven, eight, nine, ten, eleven or twelve mismatch(es) from the corresponding miRNA. In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a polynucleotide of the invention, the polynucleotide can be targeted for degradation or reduced translation, provided the miRNA in question is available. This can reduce off-target effects upon delivery of the polynucleotide. For example, if a polynucleotide of the invention is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the polynucleotide.

Conversely, miRNA binding sites can be removed from polynucleotide sequences in which they naturally occur in order to increase protein expression in specific tissues. For example, a binding site for a specific miRNA can be removed from a polynucleotide to improve protein expression in tissues or cells containing the miRNA.

In one embodiment, a polynucleotide of the invention can include at least one miRNA-binding site in the 5′UTR and/or 3′UTR in order to regulate cytotoxic or cytoprotective mRNA therapeutics to specific cells such as, but not limited to, normal and/or cancerous cells. In another embodiment, a polynucleotide of the invention can include two, three, four, five, six, seven, eight, nine, ten, or more miRNA-binding sites in the 5′-UTR and/or 3′-UTR in order to regulate cytotoxic or cytoprotective mRNA therapeutics to specific cells such as, but not limited to, normal and/or cancerous cells.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific miRNAs are involved in immunogenicity, autoimmunity, the immune-response to infection, inflammation, as well as unwanted immune response after gene therapy and tissue/organ transplantation. Immune cells specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a polynucleotide can be shut-off by adding miR-142 binding sites to the 3′-UTR of the polynucleotide, enabling more stable gene transfer in tissues and cells. miR-142 efficiently degrades exogenous polynucleotides in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of a polynucleotide of the invention can selectively repress gene expression in antigen presenting cells through miR-142 mediated degradation, limiting antigen presentation in antigen presenting cells (e.g., dendritic cells) and thereby preventing antigen-mediated immune response after the delivery of the polynucleotide. The polynucleotide is then stably expressed in target tissues or cells without triggering cytotoxic elimination.

In one embodiment, binding sites for miRNAs that are known to be expressed in immune cells, in particular, antigen presenting cells, can be engineered into a polynucleotide of the invention to suppress the expression of the polynucleotide in antigen presenting cells through miRNA mediated RNA degradation, subduing the antigen-mediated immune response. Expression of the polynucleotide is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of a polynucleotide of the invention.

To further drive the selective degradation and suppression in APCs and macrophage, a polynucleotide of the invention can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.)

miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p. MiRNA binding sites from any liver specific miRNA can be introduced to or removed from a polynucleotide of the invention to regulate expression of the polynucleotide in the liver. Liver specific miRNA binding sites can be engineered alone or further in combination with immune cell (e.g., APC) miRNA binding sites in a polynucleotide of the invention.

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p. miRNA binding sites from any lung specific miRNA can be introduced to or removed from a polynucleotide of the invention to regulate expression of the polynucleotide in the lung. Lung specific miRNA binding sites can be engineered alone or further in combination with immune cell (e.g., APC) miRNA binding sites in a polynucleotide of the invention.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p.

miRNA binding sites from any heart specific microRNA can be introduced to or removed from a polynucleotide of the invention to regulate expression of the polynucleotide in the heart. Heart specific miRNA binding sites can be engineered alone or further in combination with immune cell (e.g., APC) miRNA binding sites in a polynucleotide of the invention.

miRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p.

miRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657. miRNA binding sites from any CNS specific miRNA can be introduced to or removed from a polynucleotide of the invention to regulate expression of the polynucleotide in the nervous system. Nervous system specific miRNA binding sites can be engineered alone or further in combination with immune cell (e.g., APC) miRNA binding sites in a polynucleotide of the invention.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944.

MiRNA binding sites from any pancreas specific miRNA can be introduced to or removed from a polynucleotide of the invention to regulate expression of the polynucleotide in the pancreas. Pancreas specific miRNA binding sites can be engineered alone or further in combination with immune cell (e.g. APC) miRNA binding sites in a polynucleotide of the invention.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562.

miRNA binding sites from any kidney specific miRNA can be introduced to or removed from a polynucleotide of the invention to regulate expression of the polynucleotide in the kidney. Kidney specific miRNA binding sites can be engineered alone or further in combination with immune cell (e.g., APC) miRNA binding sites in a polynucleotide of the invention.

miRNAs that are known to be expressed in the muscle include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p. MiRNA binding sites from any muscle specific miRNA can be introduced to or removed from a polynucleotide of the invention to regulate expression of the polynucleotide in the muscle. Muscle specific miRNA binding sites can be engineered alone or further in combination with immune cell (e.g., APC) miRNA binding sites in a polynucleotide of the invention.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep-sequencing analysis (e.g., Voellenkle C et al., RNA, 2012, 18, 472-484, herein incorporated by reference in its entirety). miRNA binding sites from any endothelial cell specific.

miRNA can be introduced to or removed from a polynucleotide of the invention to regulate expression of the polynucleotide in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells. miRNA binding sites from any epithelial cell specific miRNA can be introduced to or removed from a polynucleotide of the invention to regulate expression of the polynucleotide in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy K T et al., Curr. Mol Med, 2013, 13(5), 757-764; Vidigal J A and Ventura A, Semin Cancer Biol. 2012, 22(5-6), 428-436; Goff L A et al., PLoS One, 2009, 4:e7192; Morin R D et al., Genome Res, 2008, 18, 610-621; Yoo J K et al., Stem Cells Dev. 2012, 21(11), 2049-2057, each of which is herein incorporated by reference in its entirety). MiRNAs abundant in embryonic stem cells include, but are not limited to, let-7a-2-3p, let-a-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-5481, miR-548m, miR-548n, miR-548o-3p, miR-548o-5p, miR-548p, miR-664a-3p, miR-664a-5p, miR-664b-3p, miR-664b-5p, miR-766-3p, miR-766-5p, miR-885-3p, miR-885-5p, miR-93-3p, miR-93-5p, miR-941, miR-96-3p, miR-96-5p, miR-99b-3p and miR-99b-5p. Many predicted novel miRNAs are discovered by deep sequencing in human embryonic stem cells (e.g., Morin R D et al., Genome Res, 2008, 18, 610-621; Goff L A et al., PLoS One, 2009, 4:e7192; Bar M et al., Stem cells, 2008, 26, 2496-2505, the content of each of which is incorporated herein by reference in its entirety).

In one embodiment, the binding sites of embryonic stem cell specific miRNAs can be included in or removed from the 3′UTR of a polynucleotide of the invention to modulate the development and/or differentiation of embryonic stem cells, to inhibit the senescence of stem cells in a degenerative condition (e.g. degenerative diseases), or to stimulate the senescence and apoptosis of stem cells in a disease condition (e.g. cancer stem cells).

Many miRNA expression studies are conducted to profile the differential expression of miRNAs in various cancer cells/tissues and other diseases. Some miRNAs are abnormally over-expressed in certain cancer cells and others are under-expressed. For example, miRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563, the content of each of which is incorporated herein by reference in its entirety.)

As a non-limiting example, miRNA binding sites for miRNAs that are over-expressed in certain cancer and/or tumor cells can be removed from the 3′UTR of a polynucleotide of the invention, restoring the expression suppressed by the over-expressed miRNAs in cancer cells, thus ameliorating the corresponsive biological function, for instance, transcription stimulation and/or repression, cell cycle arrest, apoptosis and cell death. Normal cells and tissues, wherein miRNAs expression is not up-regulated, will remain unaffected.

miRNA can also regulate complex biological processes such as angiogenesis (e.g., miR-132) (Anand and Cheresh Curr Opin Hematol 2011 18:171-176). In the polynucleotides of the invention, miRNA binding sites that are involved in such processes can be removed or introduced, in order to tailor the expression of the polynucleotides to biologically relevant cell types or relevant biological processes. In this context, the polynucleotides of the invention are defined as auxotrophic polynucleotides.

In some embodiments, the therapeutic window and/or differential expression (e.g., tissue-specific expression) of a polypeptide of the invention (e.g., one or more BH3 domains or a Bcl-2-like polypeptide) may be altered by incorporation of a miRNA binding site into an mRNA encoding the polypeptide. In one example, an mRNA may include one or more miRNA binding sites that are bound by miRNAs that have higher expression in one tissue type as compared to another. In another example, an mRNA may include one or more miRNA binding sites that are bound by miRNAs that have lower expression in a cancer cell as compared to a non-cancerous cell of the same tissue of origin. When present in a cancer cell that expresses low levels of such an miRNA, the polypeptide encoded by the mRNA typically will show increased expression. If the polypeptide is able to induce apoptosis, for example, by inhibiting an anti-apoptotic Bcl-2 family member and/or by activating a pro-apoptotic Bcl-2 family member, this may result in preferential cell killing of cancer cells as compared to normal cells.

Liver cancer cells (e.g., hepatocellular carcinoma cells) typically express low levels of miR-122 as compared to normal liver cells. Therefore, an mRNA encoding a polypeptide (e.g., an mRNA encoding one or more BH3 domains) that includes at least one miR-122 binding site (e.g., in the 3′-UTR of the mRNA) will typically express comparatively low levels of the polypeptide in normal liver cells and comparatively high levels of the polypeptide in liver cancer cells. If the polypeptide is able to induce apoptosis (such as one or more BH3 domains, as described herein), this can cause preferential cell killing of liver cancer cells (e.g., hepatocellular carcinoma cells) as compared to normal liver cells.

Liver cancer cells (e.g., hepatocellular carcinoma cells) typically express high levels of miR-21 as compared to normal liver cells. Therefore, an mRNA encoding a polypeptide (e.g., a Bcl-2-like polypeptide) that includes at least one miR-21 binding site (e.g., in the 3′-UTR of the mRNA) will typically express comparatively high levels of the polypeptide in normal liver cells and comparatively low levels of the polypeptide in liver cancer cells. If the polypeptide is able to inhibit apoptosis (e.g., by inhibiting activity of one or more BH3 domains, as described herein), this can further cause preferential cell killing of liver cancer cells (e.g., hepatocellular carcinoma cells) as compared to normal liver cells. For example, in normal liver cells, the Bcl-2-like-polypeptide (or BH3-trap) will be expressed and inhibit apoptosis induced by the BH3 domain(s) expressed in the normal liver cells.

In particular embodiments, the present invention contemplates the use of two or more mRNAs, wherein the first mRNA encodes one or more BH3 domains and the second mRNA encodes an inhibitor of the Bh3 domain(s) (e.g., referred to as a BH3-trap polypeptide), such as a Bcl-2-like polypeptide, or a variant or fragment thereof. In particular embodiments, when expressed in the same cell, the BH3-trap polypeptide binds the BH3 domain(s), thus preventing it from binding or inhibiting anti-apoptotic Bcl-2 family proteins present in the cell. In particular embodiments, the first mRNA encoding the BH3 domain(s) comprises one or more regulatory sequences to enhance expression in cancer cells as compared to normal cells. In particular embodiments, the second mRNA encoding the BH3-trap polypeptide comprises one or more regulatory sequences to reduce expression in cancer cells as compared to normal cells. In particular embodiments, the first mRNA comprises at least one first microRNA binding site, wherein the cognate microRNA that binds the first microRNA binding site is preferentially expressed in normal cells as compared to cancer cells. In particular embodiments, the second mRNA comprises at least one second microRNA binding site, wherein the cognate microRNA that binds the second microRNA binding site is preferentially expressed in cancer cells as compared to normal cells. Thus, the expression of the BH3 domain(s), which induces apoptosis, is increased in cancer cells as compared to normal cells, and the expression of the BH3-trap polypeptide that inhibits BH3-induced apoptosis is increased in normal cells as compared to cancer cells, thus specifically targeting cancer cells for apoptosis. In certain embodiments, the first microRNA binding site is a miR-122 binding site, and the second microRNA binding site is a miR-21 binding site. In certain instances, the present invention contemplates the use of a first mRNA that encodes one or more BH3 domains, wherein this first mRNA contains one or more miR-122 binding sites, and a second mRNA that encodes a BH3-trap polypeptide, e.g., a Bcl-2-like polypeptide, where this second mRNA contains one or more miR-21 binding sites.

As a non-limiting example, mRNAs of the invention (e.g., those encoding one or more BH3 domains) may include at least one miR-122 binding site. For example, a mRNA of the invention may include a miR-122 binding site that includes a sequence with partial or complete complementarity with a miR-122 seed sequence. In some embodiments, a miR-122 seed sequence may correspond to nucleotides 2-7 of a miR-122. In some embodiments, a miR-122 seed sequence may be 5′-GGAGUG-3′. In some embodiments, a miR-122 seed sequence may be nucleotides 2-8 of a miR-122. In some embodiments, a miR-122 seed sequence may be 5′-GGAGUGU-3′. In some embodiments, the miR-122 binding site includes a nucleotide sequence of 5′-UAUUUAGUGUGAUAAUGGCGUU-3′ (SEQ ID NO: 31) or 5′-CAAACACCAUUGUCACACUCCA-3′ (SEQ ID NO: 32) or a complement thereof. In some embodiments, inclusion of at least one miR-122 binding site in an mRNA may dampen expression of a polypeptide encoded by the mRNA in a normal liver cell as compared to other cell types that express low levels of miR-122. In other embodiments, inclusion of at least one miR-122 binding site in an mRNA may allow increased expression of a polypeptide encoded by the mRNA in a liver cancer cell (e.g., a hepatocellular carcinoma cell) as compared to a normal liver cell. In some embodiments, an mRNA that encodes one or more BH3 domains contains one or more miR-122 binding sites.

As a further non-limiting example, mRNAs of the invention, e.g., those encoding BH3-trap polypeptides, such as a Bcl-2-like polypeptide may include at least one miR-21 binding site. For example, an mRNA of the invention may include a miR-21 binding site that includes a sequence with partial or complete complementarity with a miR-21 seed sequence. In some embodiments, a miR-21 sequence may be 5′-UAGCUUAUCAGACUGAUGUUGA-3′ (SEQ ID NO: 33) or 5′-CAACACCAGUCGAUGGGCUGU-3′ (SEQ ID NO: 34) or a complement thereof. In some embodiments, a miR-21 seed sequence may correspond to nucleotides 1-8 or 2-8 of a miR-21. In some embodiments, a miR-21 seed sequence may be 5′-UAGCUUAU-3′ or 5′-AGCUUAU-3′ or a complement thereof. In other embodiments, a miR-21 seed has the sequence shown in SEQ ID NO: 106.

In some embodiments, inclusion of at least one miR-21 binding site in an mRNA may increase expression of a polypeptide encoded by the mRNA in a normal liver cell as compared to other cell types that express low levels of miR-21, such as liver cancer cells. In other embodiments, inclusion of at least one miR-21 binding site in an mRNA may allow reduced expression of a polypeptide encoded by the mRNA in a liver cancer cell (e.g., a hepatocellular carcinoma cell) as compared to a normal liver cell. In some embodiments, an mRNA that encodes a BH3-trap polypeptide (e.g., a Bcl-2-like polypeptide or variant there) contains one or more miR-21 binding sites.

In some embodiments, a polynucleotide of the invention comprises a miRNA binding site, wherein the miRNA binding site comprises one or more nucleotide sequences selected from Table 14, including one or more copies of any one or more of the miRNA binding site sequences. In some embodiments, a polynucleotide of the invention further comprises at least one, two, three, four, five, six, seven, eight, nine, ten, or more of the same or different miRNA binding sites selected from Table 14, including any combination thereof. In some embodiments, the miRNA binding site binds to miR-142 or is complementary to miR-142. In some embodiments, the miR-142 comprises SEQ ID NO: 297. In some embodiments, the miRNA binding site binds to miR-142-3p or miR-142-5p. In some embodiments the miR-142-3p comprises SEQ ID NO: 295. In some embodiments, the miR-142-5p comprises SEQ ID NO: 296. In some embodiments, the miRNA binding site comprises a nucleotide sequence at least 80%, at least 85%, at least 90%, at least 95%, or 100% identical to any of the sequences in Table 14.

TABLE 14
miR-142 Binding Sites

| SEQ ID NO. | Description | Sequence |
|---|---|---|
| 297 | miR-142 | GACAGUGCAGUCACCCAUAAAGU |
| | | AGAAAGCACUACUAACAGCACUG |
| | | GAGGGUGUAGUGUUUCCUACUU |
| | | UAUGGAUGAGUGUACUGUG |
| 295 | miR-142-3p | UGUAGUGUUUCCUACUUUAUGG |
| | | A |
| 298 | miR-142-3p | UCCAUAAAGUAGGAAACACUACA |
| | binding site | |
| 296 | miR-142-5p | CAUAAAGUAGAAAGCACUACU |
| 299 | miR-142-5p | AGUAGUGCUUUCUACUUUAUG |
| | binding site | |

In some embodiments, a miRNA binding site is inserted in the polynucleotide of the invention in any position of the polynucleotide (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the polynucleotide can be anywhere in the polynucleotide as long as the insertion of the miRNA binding site in the polynucleotide does not interfere with the translation of a functional polypeptide in the absence of the corresponding miRNA; and in the presence of the miRNA, the insertion of the miRNA binding site in the polynucleotide and the binding of the miRNA binding site to the corresponding miRNA are capable of degrading the polynucleotide or preventing the translation of the polynucleotide.

In some embodiments, a miRNA binding site is inserted in at least about 30 nucleotides downstream from the stop codon of an ORF in a polynucleotide of the invention comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the invention. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a polynucleotide of the invention.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The polynucleotides of the invention can further include this structured 5′UTR in order to enhance microRNA mediated gene regulation.

At least one miRNA binding site can be engineered into the 3′UTR of a polynucleotide of the invention. In this context, at least two, at least three, at least four, at least five, at least six, at least seven, at least eight, at least nine, at least ten, or more miRNA binding sites can be engineered into a 3′UTR of a polynucleotide of the invention. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a polynucleotide of the invention. In one embodiment, miRNA binding sites incorporated into a polynucleotide of the invention can be the same or can be different miRNA sites. A combination of different miRNA binding sites incorporated into a polynucleotide of the invention can include combinations in which more than one copy of any of the different miRNA sites are incorporated. In another embodiment, miRNA binding sites incorporated into a polynucleotide of the invention can target the same or different tissues in the body. As a non-limiting example, through the introduction of tissue-, cell-type-, or disease-specific miRNA binding sites in the 3′-UTR of a polynucleotide of the invention, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a polynucleotide of the invention. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In one embodiment, a polynucleotide of the invention can be engineered to include more than one miRNA site expressed in different tissues or different cell types of a subject. As a non-limiting example, a polynucleotide of the invention can be engineered to include miR-192 and miR-122 to regulate expression of the polynucleotide in the liver and kidneys of a subject. In another embodiment, a polynucleotide of the invention can be engineered to include more than one miRNA site for the same tissue.

In some embodiments, the therapeutic window and or differential expression associated with the polypeptide encoded by a polynucleotide of the invention can be altered with a miRNA binding site. For example, a polynucleotide encoding a polypeptide that provides a death signal can be designed to be more highly expressed in cancer cells by virtue of the miRNA signature of those cells. Where a cancer cell expresses a lower level of a particular miRNA, the polynucleotide encoding the binding site for that miRNA (or miRNAs) would be more highly expressed. Hence, the polypeptide that provides a death signal triggers or induces cell death in the cancer cell. Neighboring noncancer cells, harboring a higher expression of the same miRNA would be less affected by the encoded death signal as the polynucleotide would be expressed at a lower level due to the effects of the miRNA binding to the binding site or “sensor” encoded in the 3′UTR. Conversely, cell survival or cytoprotective signals can be delivered to tissues containing cancer and non-cancerous cells where a miRNA has a higher expression in the cancer cells—the result being a lower survival signal to the cancer cell and a larger survival signal to the normal cell.

Multiple polynucleotides can be designed and administered having different signals based on the use of miRNA binding sites as described herein.

In some embodiments, the expression of a polynucleotide of the invention can be controlled by incorporating at least one sensor sequence in the polynucleotide and formulating the polynucleotide for administration. As a non-limiting example, a polynucleotide of the invention can be targeted to a tissue or cell by incorporating a miRNA binding site and formulating the polynucleotide in a lipid nanoparticle comprising a cationic lipid, including any of the lipids described herein.

A polynucleotide of the invention can be engineered for more targeted expression in specific tissues, cell types, or biological conditions based on the expression patterns of miRNAs in the different tissues, cell types, or biological conditions. Through introduction of tissue-specific miRNA binding sites, a polynucleotide of the invention can be designed for optimal protein expression in a tissue or cell, or in the context of a biological condition.

In some embodiments, a polynucleotide of the invention can be designed to incorporate miRNA binding sites that either have 100% identity to known miRNA seed sequences or have less than 100% identity to miRNA seed sequences. In some embodiments, a polynucleotide of the invention can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the polynucleotide. In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop.

In another embodiment, a miRNA seed sequence can be incorporated in the loop of a stem loop and a miRNA binding site can be incorporated into the 5′ or 3′ stem of the stem loop.

In one embodiment, a translation enhancer element (TEE) can be incorporated on the 5′end of the stem of a stem loop and a miRNA seed can be incorporated into the stem of the stem loop. In another embodiment, a TEE can be incorporated on the 5′ end of the stem of a stem loop, a miRNA seed can be incorporated into the stem of the stem loop and a miRNA binding site can be incorporated into the 3′ end of the stem or the sequence after the stem loop. The miRNA seed and the miRNA binding site can be for the same and/or different miRNA sequences.

In one embodiment, the incorporation of a miRNA sequence and/or a TEE sequence changes the shape of the stem loop region which can increase and/or decrease translation. (see e.g, Kedde et al., “A Pumilio-induced RNA structure switch in p27-3′UTR controls miR-221 and miR-22 accessibility.” Nature Cell Biology. 2010, incorporated herein by reference in its entirety).

In one embodiment, the 5′-UTR of a polynucleotide of the invention can comprise at least one miRNA sequence. The miRNA sequence can be, but is not limited to, a 19 or 22 nucleotide sequence and/or a miRNA sequence without the seed.

In one embodiment the miRNA sequence in the 5′UTR can be used to stabilize a polynucleotide of the invention described herein.

In another embodiment, a miRNA sequence in the 5′UTR of a polynucleotide of the invention can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) in order to decrease the accessibility to the first start codon (AUG). Matsuda showed that altering the sequence around the start codon with an LNA or EJC affected the efficiency, length and structural stability of a polynucleotide. A polynucleotide of the invention can comprise a miRNA sequence, instead of the LNA or EJC sequence described by Matsuda et al, near the site of translation initiation in order to decrease the accessibility to the site of translation initiation. The site of translation initiation can be prior to, after or within the miRNA sequence. As a non-limiting example, the site of translation initiation can be located within a miRNA sequence such as a seed sequence or binding site. As another non-limiting example, the site of translation initiation can be located within a miR-122 sequence such as the seed sequence or the mir-122 binding site.

In some embodiments, a polynucleotide of the invention can include at least one miRNA in order to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a polynucleotide of the invention can be specific to the hematopoietic system. As another non-limiting example, a miRNA incorporated into a polynucleotide of the invention to dampen antigen presentation is miR-142-3p.

In some embodiments, a polynucleotide of the invention can include at least one miRNA in order to dampen expression of the encoded polypeptide in a tissue or cell of interest. As a non-limiting example, a polynucleotide of the invention can include at least one miR-122 binding site in order to dampen expression of an encoded polypeptide of interest in the liver. As another non-limiting example a polynucleotide of the invention can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a polynucleotide of the invention can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make a polynucleotide of the invention more unstable in antigen presenting cells. Non-limiting examples of these miRNAs include mir-142-5p, mir-142-3p, mir-146a-5p, and mir-146-3p.

In one embodiment, a polynucleotide of the invention comprises at least one miRNA sequence in a region of the polynucleotide that can interact with a RNA binding protein.

In some embodiments, the polynucleotide of the invention (e.g., a RNA, e.g., a mRNA) comprising (i) a sequence-optimized nucleotide sequence (e.g., an ORF) encoding a BH3 polypeptide (e.g., the wild-type sequence, functional fragment, or variant thereof) and (ii) a miRNA binding site (e.g., a miRNA binding site that binds to miR-142).

In some embodiments, the polynucleotide of the invention (e.g., BH3 polynucleotide) comprises a uracil-modified sequence encoding a polypeptide disclosed herein and a miRNA binding site disclosed herein, e.g., a miRNA binding site that binds to miR-142. In some embodiments, the uracil-modified sequence encoding a SteA-BH3 polypeptide comprises at least one chemically modified nucleobase, e.g., 5-methoxyuracil. In some embodiments, at least 95% of a type of nucleobase (e.g., uracil) in a uracil-modified sequence encoding a polypeptide (e.g., BH3) of the invention are modified nucleobases. In some embodiments, at least 95% of uricil in a uracil-modified sequence encoding a polypeptide is 5-methoxyuridine. In some embodiments, the polynucleotide comprising a nucleotide (e.g., BH3) sequence encoding a polypeptide disclosed herein (e.g., BH3) and a miRNA binding site is formulated with a delivery agent, e.g., a compound having the Formula (I), e.g., any of Compounds 1-147.

### Scaffold Polypeptides

While the mRNA constructs of the invention that encode one or more intracellular binding domains, as described herein, in many embodiments do not also encode a scaffold protein or polypeptide (since such a scaffold is not necessary for function of the domain(s) intracellularly), nevertheless in certain embodiments, an mRNA construct of the invention may encode a fusion polypeptide comprising one or more intracellular binding domains and a scaffold polypeptide. In one embodiment, the scaffold polypeptide comprises a non-antibody scaffold protein which binds to an intracellular target. In one embodiment, the scaffold polypeptide is a fibronectin domain. In another embodiment, the scaffold polypeptide is a Kunitz domain. In another embodiment, the scaffold polypeptide is a transferrin domain. In another embodiment, the scaffold polypeptide is a Stefin A polypeptide, such as a Stefin A (SteA) mutant scaffold polypeptide (described further below).

In various embodiments, an mRNA of the invention encodes a fusion polypeptide comprising a Stefin A (SteA) scaffold polypeptide, wherein the SteA scaffold polypeptide comprises one or more intracellular binding domains located at the N-terminal insertion site, the loop 1 insertion site and/or the loop 2 insertion site such that the intracellular binding domain(s) are presented on the SteA scaffold polypeptide. Stefin A (also known in the art as cystatin A) is the founding member of the cystatin family of protein inhibitors of cysteine cathepsins, which are lysosomal peptidases of the papain family. The Stefin subgroup of the cystatin family are relatively small (e.g., around 100 amino acid residues long) single domain proteins. SteA is characterized as a monomeric, single chain, single domain polypeptide of 98 amino acids long. The structure of SteA has been solved, enabling rational engineering of the protein to allow for insertion and display of intracellular binding domain amino acid sequences at defined sites. SteA contains a structural loop called “loop 1” at amino acid positions 48-50, inclusive, and a loop called “loop 2” at amino acid positions 71-79, inclusive. Both loop 1 and loop 2 are sandwiched by amino acids that form beta-sheets. Wild-type SteA is considered in the art to have one known biological activity, which is inhibition of cathepsin activity. Wild-type SteA typically interacts with cathepsins using three binding interfaces: the N-terminus, the loop 1 region, and the loop 2 region, with key contacts being made by glycine at position 4, valine at position 48, and lysine at position 73. In some embodiments, a SteA scaffold polypeptide includes one or more mutations that reduces or abrogates cathepsin inhibitory activity.

In some embodiments, a SteA scaffold polypeptide of the invention is derived from a SteA sequence (for example, a wild-type SteA sequence, for instance, human SteA), or from a derivative of SteA known in the art and/or as described below, for example, any derivative of SteA described in U.S. Pat. Nos. 8,063,019 and 8,853,131, incorporated herein by reference. Non-limiting exemplary SteA scaffold polypeptides which may be used in the compositions and methods of the invention include wild-type SteA (e.g., human SteA), STM (“Stefin A Triple Mutant,” as described, e.g., in U.S. Pat. No. 8,063,019 and in Woodman et al., J. Mol. Biol. 352: 1118-1133, 2005), SQM (“Stefin A Quadruple Mutant,” as described, e.g., in U.S. Pat. No. 8,853,131), SQT (“Stefin A Quadruple Mutant, Tracy,” as described, e.g., in U.S. Pat. No. 8,853,131 and Stadler et al., Protein Eng. Des. Sel. 24(9): 751-763, 2011), and other SteA scaffold polypeptides described in U.S. Pat. No. 8,853,131 and Hoffman et al., Protein Eng. Des. Sel. 23(5): 403-413, 2010, including SDM (“Stefin A Double Mutant”), SUC (“Stefin A Unique C-terminus”), SUM (“Stefin A Unique Middle”), SUN (“Stefin A Unique N-terminus”), or fragments thereof, including SDM-, SDM--, SQM-, SQM--, SUC-, SUC--, SUM-, SUM--, SUN-, SUN--, and SQL.

The amino acid sequence of wild-type human SteA is:

(SEQ ID NO: 53)
MIPGGLSEAKPATPEIQEIVDKVKPQLEEKTNETYGKLEAVQYKTQVVAG
TNYYIKVRAGDNKYMHLKVFKSLPGQNEDLVLTGYQVDKNKDDELTGF.

The amino acid sequence of STM is:

(SEQ ID NO: 54)
MIPWGLSEAKPATPEIQEIVDKVKPQLEEKTNETYGKLEAVQYKTQVDAG
TNYYIKVRAGDNKYMHLKVFNGPPGQNEDLVLTGYQVDKNKDDELTGF.

Compared to wild-type SteA, STM contains a G4W mutation, which disrupts the interaction of STM with cathepsins, a V48D mutation that disrupts the interaction of STM with cathepsins and reduces dimer formation through domain swapping, and a mutation to introduce a unique RsrII restriction enzyme site at codons 71-73. In some embodiments, a polynucleotide sequence encoding an intracellular binding domain may be inserted into the RsrII site of STM, thereby introducing an intracellular binding domain amino acid sequence into loop 2.

An exemplary amino acid sequence of SDM is:

(SEQ ID NO: 55)
MIPGGLSEAKPATPEIQEIVDKVKPQLEEKTNETYGKLEAVQYKTQVLAS
TNYYIKVRAGDNKYMHLKVFNGPPGQNEDLVRSGYQVDKNKDDELTGF.

SDM contains a Leu residue at position 48 as a result of an engineered NheI restriction enzyme site added to the open reading frame at codons 48-50, inclusive, as compared to wild-type SteA or STM. In some embodiments, a polynucleotide sequence encoding an intracellular binding domain may be inserted at the NheI site of SDM, thereby introducing an intracellular binding domain amino acid sequence into loop 1. SDM also contains the sequence Asn-Gly-Pro at positions 71-73 as a result of an engineered RsrII site added to the open reading frame at codons 71-73, inclusive, as compared to wild type SteA and STM. SDM also contains the sequence Arg-Ser at positions 82-83 as a result of an engineered RsrII site added to the open reading frame at codons 82-83, inclusive, as compared to wild type SteA or STM. In some embodiments, a polynucleotide sequence encoding an intracellular binding domain may be inserted between the RsrII sites of SDM, thereby replacing loop 2 with an intracellular binding domain amino acid sequence.

The amino acid sequence of SQM is:

(SEQ ID NO: 56)
MIPRGLSEAKPATPEIQEIVDKVKPQLEEKTNETYGKLEAVQYKTQVLAS
TNYYIKVRAGDNKYMHLKVFNGPPGQNEDLVRSGYQVDKNKDDELTGF.

SQM contains an Arg residue at position 4 as a result of an engineered AvrII restriction enzyme site added to the open reading frame as compared to STM or wild-type SteA. In some embodiments, a polynucleotide sequence encoding an intracellular binding domain may be inserted at the AvrII site of SQM, thereby introducing an intracellular binding domain amino acid sequence into the N-terminus of SQM. SQM also contains a Leu residue at position 48 as a result of an engineered NheI restriction enzyme site added to the open reading frame at codons 48-50, inclusive, as compared to wild-type SteA or STM. In some embodiments, a polynucleotide sequence encoding an intracellular binding domain may be inserted at the NheI site of SQM, thereby introducing an intracellular binding domain amino acid sequence into loop 1. SQM also contains the sequence Asn-Gly-Pro at positions 71-73 as a result of an engineered RsrII site added to the open reading frame at codons 71-73, inclusive, as compared to wild type SteA and STM. SQM also contains the sequence Arg-Ser at positions 82-83 as a result of an engineered RsrII site added to the open reading frame at codons 82-83, inclusive, as compared to wild type SteA or STM. In some embodiments, a polynucleotide sequence encoding an intracellular binding domain may be inserted between the RsrII sites of SQM, thereby replacing loop 2 with an intracellular binding domain amino acid sequence.

The amino acid sequence of SUC is:

(SEQ ID NO: 57)
MIPGGLSEAKPATPEIQEIVDKVKPQLEEKTNETYGKLEAVQYKTQVVAG
TNYYIKVRAGDNKYMHLKVFNGPPGQNEDLVRSGYQVDKNKDDELTGF.

SUC also contains the sequence Asn-Gly-Pro at positions 71-73 as a result of an engineered RsrII site added to the open reading frame at codons 71-73, inclusive, as compared to wild type SteA. SUC also contains the sequence Arg-Ser at positions 82-83 as a result of an engineered RsrII site added to the open reading frame at codons 82-83, inclusive, as compared to wild type SteA or STM.

The amino acid sequence of SUM is:

(SEQ ID NO: 58)
MIPGGLSEAKPATPEIQEIVDKVKPQLEEKTNETYGKLEAVQYKTQVLAS
TNYYIKVRAGDNKYMHLKVFKSLPGQNEDLVLTGYQVDKNKDDELTGF.

SUM contains a Leu residue at position 48 as a result of an engineered NheI restriction enzyme site added to the open reading frame at codons 48-50, inclusive, as compared to wild-type SteA or STM. In some embodiments, a polynucleotide sequence encoding an intracellular binding domain may be inserted at the NheI site of SUM, thereby introducing an intracellular binding domain amino acid sequence into loop 1.

The amino acid sequence of SUN is:

(SEQ ID NO: 59)
MIPRGLSEAKPATPEIQEIVDKVKPQLEEKTNETYGKLEAVQYKTQVVAG
TNYYIKVRAGDNKYMHLKVFKSLPGQNEDLVLTGYQVDKNKDDELTGF.

SUN contains an Arg residue at position 4 as a result of an engineered AvrII restriction enzyme site added to the open reading frame as compared to STM or wild-type SteA. In some embodiments, a polynucleotide sequence encoding an intracellular binding domain may be inserted into the AvrII site of SUN, thereby introducing an intracellular binding domain amino acid sequence into the N-terminus of SUN.

The amino acid sequence of SDM- is:

(SEQ ID NO: 60)
MIPGGLSEAKPATPEIQEIVDKVKPQLEEKTNETYGKLEAVQYKTQVL
ASTNYYIKVRAGDNKYMHLKVFNGPPGQNEDLVRS.

The amino acid sequence of SDM-- is:

(SEQ ID NO: 61)
MIPGGLSEAKPATPEIQEIVDKVKPQLEEKTNETYGKLEAVQYKTQVLAS
TNYYIKVRAGDNKYMHLKVFNGP.

The amino acid sequence of SQM- is:

(SEQ ID NO: 62)
MIPRGLSEAKPATPEIQEIVDKVKPQLEEKTNETYGKLEAVQYKTQVLAS
TNYYIKVRAGDNKYMHLKVFNGPPGQNEDLVRS.

The amino acid sequence of SQM-- is:

(SEQ ID NO: 63)
MIPRGLSEAKPATPEIQEIVDKVKPQLEEKTNETYGKLEAVQYKTQVLAS
TNYYIKVRAGDNKYMHLKVFNGP.

The amino acid sequence of SUC- is:

(SEQ ID NO: 64)
MIPGGLSEAKPATPEIQEIVDKVKPQLEEKTNETYGKLEAVQYKTQVVAG
TNYYIKVRAGDNKYMHLKVF NGPPGQNEDLVRS.

The amino acid sequence of SUC-- is:

(SEQ ID NO: 65)
MIPGGLSEAKPATPEIQEIVDKVKPQLEEKTNETYGKLEAVQYKTQVVAG
TNYYIKVRAGDNKYMHLKVFNGP.

The amino acid sequence of SUM- is:

(SEQ ID NO: 66)
MIPGGLSEAKPATPEIQEIVDKVKPQLEEKTNETYGKLEAVQYKTQVLAS
TNYYIKVRAGDNKYMHLKVFKSLPGQNEDLVLT.

The amino acid sequence of SUM-- is:

(SEQ ID NO: 67)
MIPGGLSEAKPATPEIQEIVDKVKPQLEEKTNETYGKLEAVQYKTQVLAS
TNYYIKVRAGDNKYMHLKVFKSL.

The amino acid sequence of SUN- is:

(SEQ ID NO: 68)
MIPRGLSEAKPATPEIQEIVDKVKPQLEEKTNETYGKLEAVQYKTQVVAG
TNYYIKVRAGDNKYMHLKVFKSLPGQNEDLVLT.

The amino acid sequence of SUN-- is:

(SEQ ID NO: 69)
MIPRGLSEAKPATPEIQEIVDKVKPQLEEKTNETYGKLEAVQYKTQVVAG
TNYYIKVRAGDNKYMHLKVFKSL.

The amino acid sequence of SQT is:

(SEQ ID NO: 70)
MIPRGLSEAKPATPEIQEIVDKVKPQLEEKTNETYGKLEAVQYKTQVLAS
TNYYIKVRAGDNKYMHLKVFNGPPGQNADRVLTGYQVDKNKDDELTGF.

SQT contains an Arg residue at position 4 as a result of an engineered AvrII restriction enzyme site added to the open reading frame as compared to STM or wild-type SteA. In some embodiments, a polynucleotide sequence encoding an intracellular binding domain may be inserted at the AvrII site of SQT, thereby introducing an intracellular binding domain amino acid sequence into the N-terminus of SQT. SQT also contains a Leu residue at position 48 as a result of an engineered NheI restriction enzyme site added to the open reading frame at codons 48-50, inclusive, as compared to wild-type SteA or STM. In some embodiments, a polynucleotide sequence encoding an intracellular binding domain may be inserted at the NheI site of SQT, thereby introducing an intracellular binding domain amino acid sequence into loop 1. SQT also contains the sequence Asn-Gly-Pro at positions 71-73 as a result of an engineered RsrII site added to the open reading frame as compared to wild-type SteA or STM. SQT also contains the sequence Ala-Asp-Arg at positions 78-80 as a result of an engineered RsrII site added to the open reading frame as compared to wild type SteA or STM. In some embodiments, a polynucleotide sequence encoding an intracellular binding domain may be inserted between the RsrII sites of SQT, thereby replacing loop 2 with an intracellular binding domain amino acid sequence.

The amino acid sequence of SQL is:

(SEQ ID NO: 71)
MIPRGLSEAKPATPEIQEIVDKVKPQLEEKTNETYGKLEAVQYKTQVLAL
ASTNYYIKVRAGDNKYMHLKVFNGPPGQNADRVLTGYQVDKNKDDELTGF.

In some embodiments, a SteA scaffold polypeptide as used in the compositions and methods of the invention comprises an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99% identity to an amino acid sequence selected from the group consisting of SEQ ID NOs: 53-71. In some embodiments, a SteA scaffold polypeptide of the invention includes an amino acid sequence selected from the group consisting of SEQ ID NOs: 53-71. In particular embodiments, a SteA scaffold polypeptide of the invention comprises an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%), identity to the amino acid sequence of SEQ ID NO: 70. In some embodiments, a SteA scaffold polypeptide of the invention comprises an amino acid sequence of SEQ ID NO: 70.

A fusion polypeptide or SteA scaffold polypeptide of the invention may be derived from any SteA scaffold polypeptide known in the art. For example, a SteA scaffold polypeptide may include one or more mutational changes, e.g., amino acid insertions, deletions or substitutions, as compared to any SteA scaffold polypeptide described herein or known in the art. In some embodiments, a SteA scaffold polypeptide may include one or more mutational changes in one or more (e.g., 1, 2, or 3) of the following three regions: the N-terminus (e.g., a mutational change in the one or more of the first 8 codons that encode the first 8 amino acids of a SteA scaffold polypeptide), the loop 1 region (e.g., a mutational change in one or more (e.g., 1, 2, 3, 4, 5, 6, 7, 8, or 9 mutational changes) of codons 46 to 54 inclusive that encode amino acids within or adjacent to loop 1 of a SteA scaffold polypeptide), and/or the loop 2 region (e.g., a mutational change in one or more (e.g., 1, 2, 3, 4, 5, 6, 7, 8, 9, 10, 11, 12, 13, 14, 15, 16, 17, or 18 mutational changes) of codons 67 to 84 inclusive that encode amino acids within or adjacent to loop 2 of a SteA scaffold polypeptide), for example, as described in U.S. Pat. No. 8,853,131, incorporated herein by reference. In some embodiments, a SteA scaffold polypeptide may include one or more mutational changes in the N-terminus as compared to a reference SteA scaffold polypeptide. In some embodiments, a SteA scaffold polypeptide may include one or more mutational changes in the loop 1 region as compared to a reference SteA scaffold polypeptide. In some embodiments, a SteA scaffold polypeptide may include one or more mutational changes in the loop 2 region as compared to a reference SteA scaffold polypeptide. In some embodiments, a SteA scaffold polypeptide may include one or more mutational changes in the N-terminus and the loop 1 region as compared to a reference SteA scaffold polypeptide. In some embodiments, a SteA scaffold polypeptide may include one or more mutational changes in the N-terminus and the loop 2 region as compared to a reference SteA scaffold polypeptide. In some embodiments, a SteA scaffold polypeptide may include one or more mutational changes in the loop 1 region and the loop 2 region as compared to a reference SteA scaffold polypeptide. In some embodiments, a SteA scaffold polypeptide may include one or more mutational changes in the N-terminus, the loop 1 region, and the loop 2 region as compared to a reference SteA scaffold polypeptide.

The effects of a mutational change, if any, on the conformational stability, expression level, secondary structure, ability to display an intracellular binding domain amino acid sequence inserted in an insertion site, or other characteristics of a SteA scaffold polypeptide can readily be determined by a person of ordinary skill in the art, for example, using methods described in U.S. Pat. Nos. 8,063,019 and 8,853,131, incorporated herein by reference. For example, in some embodiments, a SteA scaffold polypeptide retains a substantially similar conformational stability as compared to any of the SteA scaffold polypeptides described herein, for example, wild-type SteA, STM, SQM, or SQT. Conformational stability may be determined for example, by methods including but not limited to differential scanning fluorimetry, circular dichroism, spectroscopy, or other methods known in the art. Secondary structure may be determined, for example, by circular dichroism or other methods known in the art. In some embodiments, a SteA scaffold polypeptide retains a substantially similar expression level as compared to any of the SteA scaffold polypeptides described herein, for example, wild-type SteA, STM, SQM, or SQT. Expression levels may be determined, for example, by methods including but not limited to Western blot, immunohistochemistry (IHC), mass spectrometry, enzyme-linked immunosorbent assay (ELISA), or by other methods known in the art. In some embodiments, a SteA scaffold polypeptide retains a substantially similar ability to display a domain amino acid sequence as compared to any of the SteA scaffold polypeptides described herein, for example, wild-type SteA, STM, SQM, or SQT. The ability of a SteA scaffold polypeptide to display an intracellular binding domain amino acid sequence may be determined by testing whether a known binding partner of an intracellular binding domain amino acid sequence is able to physically interact with the intracellular binding domain amino acid sequence when presented in the context of a SteA scaffold polypeptide, for example, by co-immunoprecipitation, yeast two-hybrid, or other methods known in the art.

In some embodiments, a fusion polypeptide of the invention comprises a SteA scaffold polypeptide and one or more intracellular binding domains located at a N-terminal insertion site, a loop 1 insertion site, and/or a loop 2 insertion site. In some embodiments, a fusion polypeptide includes an intracellular binding domain located at an N-terminal insertion site of a SteA scaffold polypeptide. In some embodiments, an N-terminal insertion site includes one or more of positions 1-8 inclusive (e.g., position 1, 2, 3, 4, 5, 6, 7, and/or 8) of a SteA scaffold polypeptide. In particular embodiments, the N-terminal insertion site may be position 4 of a SteA scaffold polypeptide. In some embodiments, a fusion polypeptide includes an intracellular binding domain located at a loop 1 insertion site. In some embodiments, a loop 1 insertion site includes one or more of positions 46 to 54 inclusive (e.g., position 46, 47, 48, 49, 50, 51, 52, 53, and/or 54) of a SteA scaffold polypeptide. In particular embodiments, the loop 1 insertion site may include positions 48-50, e.g., position 48, 49, and/or 50 of a SteA scaffold polypeptide. In some embodiments, a fusion polypeptide includes an intracellular binding domain located at a loop 2 insertion site. In particular embodiments, the loop 2 insertion site includes one or more of positions 71-83 inclusive (e.g., position 71, 72, 73, 74, 75, 76, 77, 78, 79, 80, 81, 82, and/or 83) of a SteA scaffold polypeptide. In further embodiments, the loop 2 insertion site may include positions 71-73, 78-80, and/or 82-83 of a SteA scaffold polypeptide. In some embodiments, a SteA scaffold polypeptide may include more than one (e.g., 1, 2, 3, 4, 5, or more) intracellular binding domains located at the same insertion site (e.g., an N-terminal insertion site, a loop 1 insertion site, or a loop 2 insertion site).

In some embodiments, a fusion polypeptide comprising a SteA scaffold polypeptide may include one or more intracellular binding domains located at multiple insertion sites (e.g., 2 or 3 insertion sites) selected from an N-terminal insertion site, a loop 1 insertion site, and/or a loop 2 insertion site. In some embodiments, the same intracellular binding domain may be located at two or more insertion sites. In other embodiments, different intracellular binding domains may be located at two or more insertion sites.

In some embodiments, an intracellular binding domain amino acid sequence may include between about 1 and about 50 amino acids. For example, in some embodiments, an intracellular binding domain amino acid sequence may include, for example, 1 to 50 amino acids, 1 to 40 amino acids, 1 to 30 amino acids, 1 to 20 amino acids, 1 to 10 amino acids, or 1 to 5 amino acids. In particular embodiments, an intracellular binding domain amino acid sequence may include, for example, between about 1 and about 26 amino acids (e.g., 1, 2, 3, 4, 5, 6, 7, 8, 9, 10, 11, 12, 13, 14, 15, 16, 17, 18, 19, 20, 21, 22, 23, 24, 25, or 26 amino acids). In other particular embodiments, an intracellular binding domain may include, for example, between about 1 and about 13 amino acids (e.g., 1, 2, 3, 4, 5, 6, 7, 8, 9, 10, 11, 12, or 13 amino acids). In particular embodiments, an intracellular binding domain may include about 10 to about 40 amino acid residues, e.g., about 10, 12, 14, 16, 18, 20, 22, 24, 26, 28, 30, 32, 34, 36, 38, or 40 residues.

It is to be understood that in addition to a SteA scaffold polypeptide and one or more intracellular binding domains, a fusion polypeptide of the present invention may include additional elements, including linkers and epitope tags. Functions of a linker region can include introduction of restriction enzyme sites into the nucleotide sequence, introduction of a flexible component or space-creating region between two protein domains, or creation of an affinity tag for specific molecular interaction. A linker may be any suitable length, for example, 1, 2, 3, 4, 5, 6, 7, 8, 9, 10, or more amino acids long. An epitope tag may be included to facilitate detection and/or purification of a fusion polypeptide. Exemplary non-limiting epitope tags include FLAG, V5, HA, myc, GFP, and His.

### Bel-2-Like Polypeptides

The present invention also includes mRNAs encoding a polypeptide that inhibits a BH3 domain encoded by constructs described herein. In some embodiments, an mRNA of the invention may encode one or more Bcl-2-like polypeptides or a variant or fragment thereof. In particular embodiments, an mRNA of the invention may encode a prosurvival Bcl-2-like polypeptide, such as Bcl-2, Bcl-X_{L}, Bcl-w, Mcl-1 or A1 polypeptide, or a variant or fragment thereof. Structural studies have shown that the hydrophobic face of the amphipathic helix present in BH3 domains inserts into a hydrophobic groove formed by the BH1, BH2 and BH3 domains of the prosurvival Bcl-2-like polypeptides, such as Bcl-2, Bcl-X_{L}, Bcl-w, Mcl-1 and A1, thus neutralizing the prosurvival Bcl-2-like polypeptides. In particular embodiments, the Bcl-2-like polypeptides and variants thereof comprise BH1, BH2 and BH3 domains. In particular embodiments, variants may include one or more N-terminal or C-terminal deletion. For example, in particular embodiments, soluble, monomeric prosurvival Bcl-2-like polypeptides have a deletion of their hydrophobic C-terminal domain. In certain embodiments, Mcl-1 and other Bcl-2-like polypeptides have a deletion of an N-terminal PEST region. In some embodiments, the Bcl-2-like polypeptide is a human polypeptide. In other embodiments, the Bcl-2-like polypeptide may be from a non-human species, e.g., Caenorhabditis elegans, rodents (e.g., mice and rats), or non-human primates. Without wishing to be bound by theory, it is believed that when expressed in the same cell, the exogenous Bcl-2-like polypeptide or variant thereof binds to the BH3 domain of the BH3 fusion polypeptide, thus sequestering it and preventing it from inducing apoptosis (see, for example, Day et al., J. Mol. Biol. 380:958-971, 2008).

In some embodiments, a Bcl-2-like polypeptide or variant or fragment thereof may include an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to the amino acid sequence of a human Bcl-2, Bcl-X_{L}, Bcl-w, Mcl-1 or A1 polypeptide, the amino acid sequences of which are shown in SEQ ID NOs: 38-42, respectively.

In particular embodiments, a Bcl-2-like polypeptide variant is a soluble Bcl-2-like polypeptide variant, such as a Bcl-2 polypeptide comprising a C-terminal truncation (e.g., deletion of the C-terminal 22-32 amino acid residues, e.g., the C-terminal 22 or 43 amino acid residues), a Bcl-X_{L} polypeptide comprising a C-terminal truncation (e.g., deletion of the C-terminal 24 amino acid residues), a Bcl-w polypeptide comprising a C-terminal truncation (e.g., deletion of the C-terminal 29 amino acid residues), a Mcl-1 polypeptide comprising C-terminal and N-terminal truncations (e.g., deletion of the C-terminal 23 amino acid residues and the N-terminal 151 amino acid residues), or an A1 polypeptide comprising a C-terminal truncation (e.g., deletion of the C-terminal 20 amino acid residues). In particular embodiments, a Bcl-2-like polypeptide may in addition or alternatively comprise one or more amino acid substitutions as compared to its corresponding wild type Bcl-2-like polypeptide. In certain embodiments, a variant includes a Bcl-2 polypeptide having one or more C29S, D34A, or A128E amino acid substitutions. In certain embodiments, a variant includes a Bcl-X_{L} polypeptide having a D61A amino acid substitution. In certain embodiments, BH3-trap polypeptides (e.g., Bcl-2-like polypeptides) of the present invention include: Mcl-1 del.N/C; Bcl-w (C29S/A128E); Bcl-2 (D34A) del.C32; Bcl-X_{L} del.C24, Mcl-1 del.N/C(2010), and Bcl-xL (D61A) del.C24 (the amino acid sequences of which are shown in SEQ ID NOs: 94-99, respectively) and wild type Bcl-2-like polypeptides. Illustrative soluble monomeric prosurvival proteins are also described in Chen et al., Molecular Cell (2005) 17, 393-403, which is hereby incorporated by reference in its entirety.

In some embodiments, a Bcl-2-like polypeptide or variant thereof as used herein may include an amino acid sequence having at least about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to any one of any one of SEQ ID NOs: 38-42. In some embodiments, the Bcl-2-like polypeptide is encoded by an mRNA sequence selected from the group consisting of SEQ ID NOs: 78-105.

In some embodiments, a Bcl-2-like polypeptide may be able to inhibit apoptosis induced by a BH3 domain polypeptide. A person of ordinary skill in the art can readily determine if a Bcl-2-like polypeptide is able to inhibit BH3-induced apoptosis using a variety of methods, for example, caspase activation assays (e.g., caspase-3/7 activation assays), stains and dyes (e.g., CELLTOX™, MITOTRACKER® Red, propidium iodide, and YOYO3), cell viability assays, cell morphology, and PARP-1 cleavage. In particular embodiments, the Bcl-2-like polypeptide is Bcl-2, Bcl-x_{L}, Bcl-w, Mcl-1 or A1. In some embodiments, the Bcl-2-like polypeptide is a functional variant selected from Mcl-1del.N/C, Bcl-2 (C295/A128E), Bcl-2 (D34A) del.C32, Bcl-xL del.C24, Mcl-1 del.N/C(2010), and Bel-xL (D61A) del.C24.

The mRNA constructs encoding the Bcl-2-like polypeptides can be used in combination with an mRNA construct encoding one or more BH3 domains by co-transfection of both constructs into cells. Alternatively, the Bcl-2-like polypeptide constructs can be introduced into cells as a single agent, wherein they can act to inhibit the activity of endogenous BH3 domains.

### Anti-MCL1 Constructs

The present invention also includes mRNAs encoding a polypeptide that targets MCL1, referred to herein as anti-MCL1 constructs, which can be used in combination with an mRNA construct encoding one or more BH3 domains to synergistically promote apoptosis. Example 5 describes in detail anti-MCL1 constructs that exhibit synergistic pro-apoptotic effects when used in combination with an SQT-BH3 construct. The anti-MCL1 constructs can similarly be used in combination with the single BH3 domain or multimer BH3 domain mRNA constructs described herein. While not intending to be limited by mechanism, it is thought that by neutralizing MCL1 in tumor cells, the tumor then reverts to sole reliance on BCLXL, BCL2 and/or other prosurvival members of the family as the prosurvival mechanism/pathway. Accordingly, use of BH3-domain encoded mRNA constructs, which specifically destroys BCLXL, BCL2 and/or other prosurvival members of the family then leads to better tumor killing when used in combination with an anti-MCL1 agent.

Non-limiting examples of sequences that can be used in anti-MCL1 constructs are shown in SEQ ID NOs: 107-116 (with an epitope tag) and in SEQ ID NOs: 117-126 (without an epitope tag). Nucleotide sequences encoding the open reading frames of SEQ ID NOs: 107-116 are shown in SEQ ID NOs: 127-136, respectively. Sequences that target MCL1 also have been described in the art (see e.g., Lee, E. F. et al. (2008) J. Cell. Biol. 180:341-355; Foight, G. W. et al. (2014) ACS Chem. Biol. 9:1962-1968; Placzek, W. J. et al. (2011) J. Biol. Chem. 286:39829-39835). In one embodiment, an anti-MCL1 construct comprises a mutated Bim BH3 domain, such as a mutant Bim BH3 domain having two alanine substitutions, as shown in SEQ ID NO: 137. An anti-MCL1 construct can comprise a single mutated Bim BH3 domain, or multiple copies (e.g., 2, 3, 4) of the mutated Bim BH3 domain.

An anti-MCL1 construct also can encode one or more copies of a linker sequence, such as a protease-sensitive peptide linker sequence, a cleavable-linker sequence and the like. For example, in a construct containing multiple copies of a polypeptide domain, a sequence encoding a protease-sensitive linker can be located between each of the sequences encoding the polypeptide domain. In one embodiment, the cleavable linker is an F2A linker (e.g., having the amino acid sequence shown in SEQ ID NO: 138). In other embodiments, the cleavable linker is a T2A linker (e.g., having the amino acid sequence shown in SEQ ID NO: 139), a P2A linker (e.g., having the amino acid sequence shown in SEQ ID NO: 140) or an E2A linker (e.g., having the amino acid sequence shown in SEQ ID NO: 141). The skilled artisan will appreciate that other art-recognized linkers may be suitable for use in the constructs of the invention (e.g., encoded by the polynucleotides of the invention). The skilled artisan will likewise appreciate that other multicistronic constructs may be suitable for use in the invention. In exemplary embodiments, the construct design yields approximately equimolar amounts of intrabody and/or domain thereof encoded by the constructs of the invention.

Furthermore, an anti-MCL1 construct can include one or more microRNA binding sites. Such binding sites are described hereinbefore. For example, in one embodiment, an anti-MCL1 construct includes a miR122 binding site.

An anti-MCL1 construct also can include an epitope tag, such as a FLAG, His or V5 epitope tag.

In other embodiments, an anti-MCL1 construct comprises a scaffold polypeptide. Thus, the construct can encode a fusion polypeptide of the scaffold polypeptide and the anti-MCL1 polypeptide(s). Suitable scaffold polypeptides include the SteA scaffolds described herein, such as an SQT scaffold. The amino acid sequence of a non-limiting example of an SQT scaffold/anti-MCL1 fusion polypeptide is shown in SEQ ID NO: 107, in which a single mutated Bim BH3 domain has been inserted into the N-terminal loop of the SQT scaffold protein. The nucleotide sequence encoding this ORF used in the mRNA construct is shown in SEQ ID NO: 127. The amino acid sequence of this ORF without the V5 epitope tag is shown in SEQ ID NO: 117.

For use of an anti-MCL1 construct in combination with an mRNA construct encoding one or more BH3 domains, both constructs can be incorporated into the same mmRNA construct and introduced into cells as a single construct. Alternatively, the two mmRNAs can be prepared as two separate constructs and they can be used in combination by introducing both constructs into the same cells. Either or both can be delivered to cells in a lipid nanoparticle as described herein.

### Screening of BH3 Domain Libraries

In one embodiment, a BH3 domain(s) of interest is selected by screening a library of BH3 domains. In certain embodiments, the library can be a library of BH3 domains that are presented on a scaffold, such as a SteA scaffold fusion polypeptide. That is, a library of nucleotides encoding BH3 domains can be incorporated into mRNAs encoding the SteA scaffold fusion polypeptide, e.g., at the N-terminal insertion site, at the loop 1 insertion site and/or at the loop 2 insertion site, and the resultant BH3 domain library can be screened for a BH3 domains having the desired binding property of interest (e.g., apoptotic ability).

The library of BH3 domains can be, for example, a library of mutated versions of known BH3 domains or can be a library of randomly generated polypeptides, for example having a BH3 domain consensus sequence.

In one embodiment, the library is a library of BH3 domains having a BH3 domain consensus sequence. For example, a library of polypeptides having the amino acid sequence of X₁X₂X₃X₄X₅X₆X₇X₈X₉DX₁₀X₁₁X₁₂, wherein X₁, X₅, X₈, and X₁₁ are, independently, any hydrophobic residue, X₂ and X₉ are, independently, Gly, Ala, or Ser, X₃, X₄, X₆, and X₇ are, independently, any amino acid residue, X₁₀ is Asp or Glu, and X₁₂ is Asn, His, Asp, or Tyr, can be generated and screened for a BH3 domain having the desired functional property, such as activation of apoptosis. In some embodiments, a hydrophobic residue is Leu, Ala, Val, Ile, Pro, Phe, Met or Trp. In some embodiments, X₅ is Leu.

In one embodiment, a library of single BH3 domains is screened. In another embodiment, a library of multiple BH3 domains (e.g., constructed similar to the multimer BH3 constructs described herein) is screened. In yet another embodiment, a library of BH3 domains presented on a scaffold, as part of a scaffold-BH3 domain fusion polypeptide, is screened. In one example of this latter embodiment, the library of BH3 domains is presented on a SteA scaffold fusion protein and screened for desired binding and/or functional properties. In another embodiment, the library of BH3 domains is screened using a different expression system, such as phage display, yeast display or other library expression system well-established in the art, a BH3 domain is selected having the desired binding and/or functional properties, the BH3 domain sequence is determined and then a nucleotide sequence encoding the selected BH3 domain sequence is introduced into an mRNA encoding a SteA scaffold fusion polypeptide, e.g., at the N-terminal insertion site, the loop insertion site and/or the loop 2 insertion site such that the selected BH3 domain can be presented by the SteA scaffold fusion polypeptide. General methodologies for screening libraries using scaffold proteins in systems such as phage display are described in, for example, PCT Publication WO 2014/125290, the contents of which is incorporated herein in its entirety.

### Nanoparticles

The mRNAs of the invention may be formulated in nanoparticles or other delivery vehicles, e.g., to protect them from degradation when delivered to a subject. Illustrative nanoparticles are described in Panyam, J. & Labhasetwar, V. Adv. Drug Deliv. Rev. 55, 329-347 (2003) and Peer, D. et al. Nature Nanotech. 2, 751-760 (2007). In certain embodiments, an mRNA of the invention is encapsulated within a nanoparticle. In particular embodiments, a nanoparticle is a particle having at least one dimension (e.g., a diameter) less than or equal to 1000 nM, less than or equal to 500 nM or less than or equal to 100 nM. In particular embodiments, a nanoparticle includes a lipid. Lipid nanoparticles include, but are not limited to, liposomes and micelles. Any of a number of lipids may be present, including cationic and/or ionizable lipids, anionic lipids, neutral lipids, amphipathic lipids, PEGylated lipids, and/or structural lipids. Such lipids can be used alone or in combination. In particular embodiments, a lipid nanoparticle comprises one or more mRNAs described herein, e.g., a mRNA encoding one or more BH3 domains and/or a mRNA encoding a BH3-trap polypeptide.

In some embodiments, the lipid nanoparticle formulations of the mRNAs described herein may include one or more (e.g., 1, 2, 3, 4, 5, 6, 7, or 8) cationic and/or ionizable lipids. Such cationic lipids include, but are not limited to, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3-β-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic and/or ionizable lipids can be used, such as, e.g., LIPOFECTIN® (including DOTMA and DOPE, available from GIBCO/BRL), and LIPOFECTAMINE® (including DOSPA and DOPE, available from GIBCO/BRL). KL10, KL22, and KL25 are described, for example, in U.S. Pat. No. 8,691,750, which is incorporated herein by reference in its entirety. In particular embodiments, the lipid is DLin-MC3-DMA or DLin-KC2-DMA.

Anionic lipids suitable for use in lipid nanoparticles of the invention include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

Neutral lipids suitable for use in lipid nanoparticles of the invention include, but are not limited to, diacylphosphatidylcholine, diacylphosphatidylethanolamine, ceramide, sphingomyelin, dihydrosphingomyelin, cephalin, and cerebrosides. Lipids having a variety of acyl chain groups of varying chain length and degree of saturation are available or may be isolated or synthesized by well-known techniques. Additionally, lipids having mixtures of saturated and unsaturated fatty acid chains can be used. In some embodiments, the neutral lipids used in the invention are DOPE, DSPC, DPPC, POPC, or any related phosphatidylcholine. In some embodiments, the neutral lipid may be composed of sphingomyelin, dihydrosphingomyeline, or phospholipids with other head groups, such as serine and inositol.

In some embodiments, amphipathic lipids are included in nanoparticles of the invention. Exemplary amphipathic lipids suitable for use in nanoparticles of the invention include, but are not limited to, sphingolipids, phospholipids, and aminolipids. In some embodiments, a phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,1,2-dioleoyl-sn-glycero-3-phosphoethanolamine (DOPE), 1,2-diphytanoyl-sn-glycero-3-phosphoethanolamine (ME 16.0 PE),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG), and sphingomyelin.
   Other phosphorus-lacking compounds, such as sphingolipids, glycosphingolipid families, diacylglycerols, and β-acyloxyacids, may also be used. Additionally, such amphipathic lipids can be readily mixed with other lipids, such as triglycerides and sterols.

In some embodiments, the lipid component of a nanoparticle of the invention may include one or more PEGylated lipids. A PEGylated lipid (also known as a PEG lipid or a PEG-modified lipid) is a lipid modified with polyethylene glycol. The lipid component may include one or more PEGylated lipids. A PEGylated lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEGylated lipid may be PEG-c-DOMG, PEG-DMG, PEG-DLPE, PEG-DMPE, PEG-DPPC, or a PEG-DSPE lipid.

A lipid nanoparticle of the invention may include one or more structural lipids. Exemplary, non-limiting structural lipids that may be present in the lipid nanoparticles of the invention include cholesterol, fecosterol, sitosterol, campesterol, stigmasterol, brassicasterol, ergosterol, tomatidine, tomatine, ursolic acid, or alpha-tocopherol).

In some embodiments, one or more mRNA of the invention may be formulated in a lipid nanoparticle having a diameter from about 1 nm to about 900 nm, e.g., about 1 nm to about 100 nm, about 1 nm to about 200 nm, about 1 nm to about 300 nm, about 1 nm to about 400 nm, about 1 nm to about 500 nm, about 1 nm to about 600 nm, about 1 nm to about 700 nm, about 1 nm to 800 nm, about 1 nm to about 900 nm. In some embodiments, the nanoparticle may have a diameter from about 10 nm to about 300 nm, about 20 nm to about 200 nm, about 30 nm to about 100 nm, or about 40 nm to about 80 nm. In some embodiments, the nanoparticle may have a diameter from about 30 nm to about 300 nm, about 40 nm to about 200 nm, about 50 nm to about 150 nm, about 70 to about 110 nm, or about 80 nm to about 120 nm. In one embodiment, an mRNA may be formulated in a lipid nanoparticle having a diameter from about 10 to about 100 nm including ranges in between such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm, and/or about 90 to about 100 nm. In one embodiment, an mRNA may be formulated in a lipid nanoparticle having a diameter from about 30 nm to about 300 nm, about 40 nm to about 200 nm, about 50 nm to about 150 nm, about 70 to about 110 nm, or about 80 nm to about 120 nm including ranges in between.

In some embodiments, a lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, or greater than 950 nm.

In some embodiments, the particle size of the lipid nanoparticle may be increased and/or decreased. The change in particle size may be able to help counter a biological reaction such as, but not limited to, inflammation, or may increase the biological effect of the mRNA delivered to a patient or subject.

In certain embodiments, it is desirable to target a nanoparticle, e.g., a lipid nanoparticle, of the invention using a targeting moiety that is specific to a cell type and/or tissue type. In some embodiments, a nanoparticle may be targeted to a particular cell, tissue, and/or organ using a targeting moiety. In particular embodiments, a nanoparticle comprises one or more mRNA described herein and a targeting moiety. Exemplary non-limiting targeting moieties include ligands, cell surface receptors, glycoproteins, vitamins (e.g., riboflavin) and antibodies (e.g., full-length antibodies, antibody fragments (e.g., Fv fragments, single chain Fv (scFv) fragments, Fab′ fragments, or F(ab′)2 fragments), single domain antibodies, camelid antibodies and fragments thereof, human antibodies and fragments thereof, monoclonal antibodies, and multispecific antibodies (e.g, bispecific antibodies)). In some embodiments, the targeting moiety may be a polypeptide. The targeting moiety may include the entire polypeptide (e.g., peptide or protein) or fragments thereof. A targeting moiety is typically positioned on the outer surface of the nanoparticle in such a manner that the targeting moiety is available for interaction with the target, for example, a cell surface receptor. A variety of different targeting moieties and methods are known and available in the art, including those described, e.g., in Sapra et al., Prog. Lipid Res. 42(5):439-62, 2003 and Abra et al., J. Liposome Res. 12:1-3, 2002.

In some embodiments, a lipid nanoparticle (e.g., a liposome) may include a surface coating of hydrophilic polymer chains, such as polyethylene glycol (PEG) chains (see, e.g., Allen et al., Biochimica et Biophysica Acta 1237: 99-108, 1995; DeFrees et al., Journal of the American Chemistry Society 118: 6101-6104, 1996; Blume et al., Biochimica et Biophysica Acta 1149: 180-184, 1993; Klibanov et al., Journal of Liposome Research 2: 321-334, 1992; U.S. Pat. No. 5,013,556; Zalipsky, Bioconjugate Chemistry 4: 296-299, 1993; Zalipsky, FEBS Letters 353: 71-74, 1994; Zalipsky, in Stealth Liposomes Chapter 9 (Lasic and Martin, Eds) CRC Press, Boca Raton Fla., 1995. In one approach, a targeting moiety for targeting the lipid nanoparticle is linked to the polar head group of lipids forming the nanoparticle. In another approach, the targeting moiety is attached to the distal ends of the PEG chains forming the hydrophilic polymer coating (see, e.g., Klibanov et al., Journal of Liposome Research 2: 321-334, 1992; Kirpotin et al., FEBS Letters 388: 115-118, 1996).

Standard methods for coupling the targeting moiety or moieties may be used. For example, phosphatidylethanolamine, which can be activated for attachment of targeting moieties, or derivatized lipophilic compounds, such as lipid-derivatized bleomycin, can be used. Antibody-targeted liposomes can be constructed using, for instance, liposomes that incorporate protein A (see, e.g., Renneisen et al., J. Bio. Chem., 265:16337-16342, 1990 and Leonetti et al., Proc. Natl. Acad. Sci. (USA), 87:2448-2451, 1990. Other examples of antibody conjugation are disclosed in U.S. Pat. No. 6,027,726. Examples of targeting moieties can also include other polypeptides that are specific to cellular components, including antigens associated with neoplasms or tumors. Polypeptides used as targeting moieties can be attached to the liposomes via covalent bonds (see, for example Heath, Covalent Attachment of Proteins to Liposomes, 149 Methods in Enzymology 111-119 (Academic Press, Inc. 1987)). Other targeting methods include the biotin-avidin system.

In some embodiments, a lipid nanoparticle of the invention includes a targeting moiety that targets the lipid nanoparticle to a cell including, but not limited to, hepatocytes, colon cells, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells (including primary tumor cells and metastatic tumor cells). In particular embodiments, the targeting moiety targets the lipid nanoparticle to a hepatocyte. In other embodiments, the targeting moiety targets the lipid nanoparticle to a colon cell. In some embodiments, the targeting moiety targets the lipid nanoparticle to a liver cancer cell (e.g., a hepatocellular carcinoma cell) or a colorectal cancer cell (e.g., a primary tumor or a metastasis).

Delivery Agents

a. Lipid Compound

The present disclosure provides pharmaceutical compositions with advantageous properties. In particular, the present application provides pharmaceutical compositions comprising:

(a) a polynucleotide comprising a nucleotide sequence encoding a polypeptide (e.g., SteA-BH3); and

(b) a lipid compound having the formula (I)

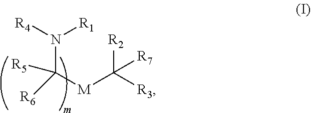

wherein

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and m is selected from 5, 6, 7, 8, 9, 10, 11, 12, and 13,

or salts or stereoisomers thereof, wherein alkyl and alkenyl groups can be linear or branched.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In another embodiments, another subset of compounds of Formula (I) includes those in which R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In yet another embodiments, another subset of compounds of Formula (I) includes those in which R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In still another embodiments, another subset of compounds of Formula (I) includes those in which

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In yet another embodiments, another subset of compounds of Formula (I) includes those in which

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is —(CH₂)ₙQ or —(CH₂)ₙCHQR, where Q is —N(R)₂, and n is selected from 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In still another embodiments, another subset of compounds of Formula (I) includes those in which

R₁ is selected from the group consisting of C₅₋₂₀ alkyl, C₂₀ alkenyl, —R*YR″, —YR″, and —R″M′ R′;

R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In certain embodiments, a subset of compounds of Formula (I) includes those of Formula (IA):

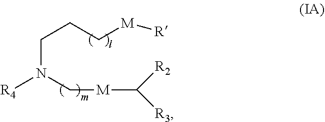

or a salt or stereoisomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, or —NHC(O)N(R)₂; M and M′ are independently selected from —C(O)O—, —OC(O)—, —C(O)N(R′)—, —P(O)(OR′)O—, an aryl group, and a heteroaryl group; and

R₂ and R₃ are independently selected from the group consisting of H, C₁₋₁₄ alkyl, and C₂₋₁₄ alkenyl.

In certain embodiments, a subset of compounds of Formula (I) includes those of Formula (II):

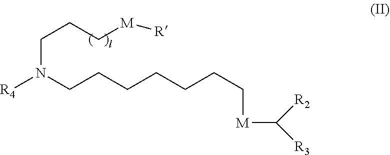

or a salt or stereoisomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, or —NHC(O)N(R)₂; M and M′ are independently selected from —C(O)O—, —OC(O)—, —C(O)N(R′)—, —P(O)(OR′)O—, an aryl group, and a heteroaryl group; and

R₂ and R₃ are independently selected from the group consisting of H, C₁₋₁₄ alkyl, and C₂₋₁₄ alkenyl.

In some embodiments, the compound of formula (I) is of the formula (IIa),

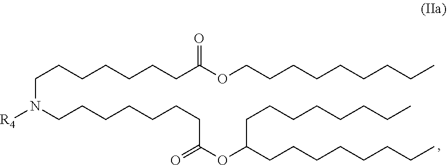

or a salt thereof, wherein R₄ is as described above.

In some embodiments, the compound of formula (I) is of the formula (IIb),

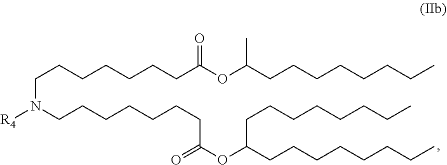

or a salt thereof, wherein R₄ is as described above.

In some embodiments, the compound of formula (I) is of the formula (IIc),

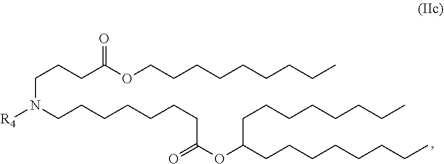

or a salt thereof, wherein R₄ is as described above.

In some embodiments, the compound of formula (I) is of the formula (IIe):

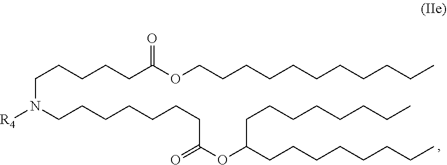

or a salt thereof, wherein R₄ is as described above.

In some embodiments, the compound of formula (IIa), (IIb), (IIc), or (IIe) comprises an R₄ which is selected from —(CH₂)ₙQ and —(CH₂)ₙCHQR, wherein Q, R and n are as defined above.

In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle, wherein R is as defined above. In some aspects, n is 1 or 2. In some embodiments, Q is OH, —NHC(S)N(R)₂, or —NHC(O)N(R)₂.

In some embodiments, the compound of formula (I) is of the formula (IId),

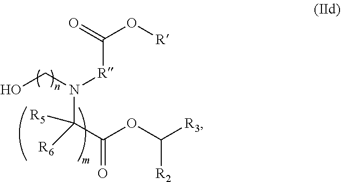

or a salt thereof, wherein R₂ and R₃ are independently selected from the group consisting of C₅₋₁₄ alkyl and C₅₋₁₄ alkenyl, n is selected from 2, 3, and 4, and R′, R″, R₅, R₆ and m are as defined above.

In some aspects of the compound of formula (IId), R₂ is C₈ alkyl. In some aspects of the compound of formula (IId), R₃ is C₅-C₉ alkyl. In some aspects of the compound of formula (IId), m is 5, 7, or 9. In some aspects of the compound of formula (IId), each R₅ is H. In some aspects of the compound of formula (IId), each R₆ is H.

In another aspect, the present application provides a lipid composition (e.g., a lipid nanoparticle (LNP)) comprising: (1) a compound having the formula (I); (2) optionally a helper lipid (e.g. a phospholipid); (3) optionally a structural lipid (e.g. a sterol); (4) optionally a lipid conjugate (e.g. a PEG-lipid); and (5) optionally a quaternary amine compound. In exemplary embodiments, the lipid composition (e.g., LNP) further comprises a polynucleotide encoding a polypeptide (e.g., SteA-BH3 polypeptide), e.g., a polynucleotide encapsulated therein.

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms).

The notation “C₁₋₁₄ alkyl” means a linear or branched, saturated hydrocarbon including 1-14 carbon atoms. An alkyl group can be optionally substituted.

As used herein, the term “alkenyl” or “alkenyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond.

The notation “C₂₋₁₄ alkenyl” means a linear or branched hydrocarbon including 2-14 carbon atoms and at least one double bond. An alkenyl group can include one, two, three, four, or more double bonds. For example, C₁₈ alkenyl can include one or more double bonds. A C₁₈ alkenyl group including two double bonds can be a linoleyl group. An alkenyl group can be optionally substituted.

As used herein, the term “carbocycle” or “carbocyclic group” means a mono- or multi-cyclic system including one or more rings of carbon atoms. Rings can be three, four, five, six, seven, eight, nine, ten, eleven, twelve, thirteen, fourteen, or fifteen membered rings.

The notation “C₃₋₆ carbocycle” means a carbocycle including a single ring having 3-6 carbon atoms. Carbocycles can include one or more double bonds and can be aromatic (e.g., aryl groups). Examples of carbocycles include cyclopropyl, cyclopentyl, cyclohexyl, phenyl, naphthyl, and 1,2-dihydronaphthyl groups. Carbocycles can be optionally substituted.

As used herein, the term “heterocycle” or “heterocyclic group” means a mono- or multi-cyclic system including one or more rings, where at least one ring includes at least one heteroatom. Heteroatoms can be, for example, nitrogen, oxygen, or sulfur atoms. Rings can be three, four, five, six, seven, eight, nine, ten, eleven, or twelve membered rings. Heterocycles can include one or more double bonds and can be aromatic (e.g., heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. Heterocycles can be optionally substituted.

As used herein, a “biodegradable group” is a group that can facilitate faster metabolism of a lipid in a subject. A biodegradable group can be, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group.

As used herein, an “aryl group” is a carbocyclic group including one or more aromatic rings. Examples of aryl groups include phenyl and naphthyl groups.

As used herein, a “heteroaryl group” is a heterocyclic group including one or more aromatic rings. Examples of heteroaryl groups include pyrrolyl, furyl, thiophenyl, imidazolyl, oxazolyl, and thiazolyl. Both aryl and heteroaryl groups can be optionally substituted. For example, M and M′ can be selected from the non-limiting group consisting of optionally substituted phenyl, oxazole, and thiazole. In the formulas herein, M and M′ can be independently selected from the list of biodegradable groups above.

Alkyl, alkenyl, and cyclyl (e.g., carbocyclyl and heterocyclyl) groups can be optionally substituted unless otherwise specified. Optional substituents can be selected from the group consisting of, but are not limited to, a halogen atom (e.g., a chloride, bromide, fluoride, or iodide group), a carboxylic acid (e.g., —C(O)OH), an alcohol (e.g., a hydroxyl, —OH), an ester (e.g., —C(O)OR or —OC(O)R), an aldehyde (e.g., —C(O)H), a carbonyl (e.g., —C(O)R, alternatively represented by C═O), an acyl halide (e.g., —C(O)X, in which X is a halide selected from bromide, fluoride, chloride, and iodide), a carbonate (e.g., —OC(O)OR), an alkoxy (e.g., —OR), an acetal (e.g., —C(OR)₂R″″, in which each OR are alkoxy groups that can be the same or different and R″″ is an alkyl or alkenyl group), a phosphate (e.g., P(O)₄³⁻), a thiol (e.g., —SH), a sulfoxide (e.g., —S(O)R), a sulfinic acid (e.g., —S(O)OH), a sulfonic acid (e.g., —S(O)₂OH), a thial (e.g., —C(S)H), a sulfate (e.g., S(O)₄²⁻), a sulfonyl (e.g., —S(O)₂—), an amide (e.g., —C(O)NR₂, or —N(R)C(O)R), an azido (e.g., —N₃), a nitro (e.g., —NO₂), a cyano (e.g., —CN), an isocyano (e.g., —NC), an acyloxy (e.g., —OC(O)R), an amino (e.g., —NR₂, —NRH, or —NH₂), a carbamoyl (e.g., —OC(O)NR₂, —OC(O)NRH, or —OC(O)NH₂), a sulfonamide (e.g., —S(O)₂NR₂, —S(O)₂NRH, —S(O)₂NH₂, —N(R)S(O)₂R, —N(H)S(O)₂R, —N(R)S(O)₂H, or —N(H)S(O)₂H), an alkyl group, an alkenyl group, and a cyclyl (e.g., carbocyclyl or heterocyclyl) group.

In any of the preceding, R is an alkyl or alkenyl group, as defined herein. In some embodiments, the substituent groups themselves can be further substituted with, for example, one, two, three, four, five, or six substituents as defined herein. For example, a C₁₋₆ alkyl group can be further substituted with one, two, three, four, five, or six substituents as described herein.

The compounds of any one of formulae (I), (IA), (II), (IIa), (IIb), (IIc), (IId), and (IIe) include one or more of the following features when applicable.

In some embodiments, R₄ is selected from the group consisting of a C₃₋₆ carbocycle, —(CH₂)ₙQ, —(CH₂)ₙCHQR, —CHQR, and —CQ(R)₂, where Q is selected from a C₃₋₆ carbocycle, 5- to 14-membered aromatic or non-aromatic heterocycle having one or more heteroatoms selected from N, O, S, and P, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5.

In another embodiment, R₄ is selected from the group consisting of a C₃₋₆ carbocycle, —(CH₂)ₙQ, —(CH₂)ₙCHQR, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —C(R)N(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, and each n is independently selected from 1, 2, 3, 4, and 5.

In another embodiment, R₄ is selected from the group consisting of a C₃₋₆ carbocycle, —(CH₂)ₙQ, —(CH₂)ₙCHQR, —CHQR, and —CQ(R)₂, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —C(R)N(R)₂C(O)OR, and each n is independently selected from 1, 2, 3, 4, and 5; and when Q is a 5- to 14-membered heterocycle and (i) R₄ is —(CH₂)ₙQ in which n is 1 or 2, or (ii) R4 is —(CH₂)ₙCHQR in which n is 1, or (iii) R₄ is —CHQR, and —CQ(R)₂, then Q is either a 5- to 14-membered heteroaryl or 8- to 14-membered heterocycloalkyl.

In another embodiment, R₄ is selected from the group consisting of a C₃₋₆ carbocycle, —(CH₂)ₙQ, —(CH₂)ₙCHQR, —CHQR, and —CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —C(R)N(R)₂C(O)OR, and each n is independently selected from 1, 2, 3, 4, and 5.

In another embodiment, R₄ is unsubstituted C₁₋₄ alkyl, e.g., unsubstituted methyl.

In certain embodiments, the disclosure provides a compound having the Formula (I), wherein R4 is —(CH₂)ₙQ or —(CH₂)ₙCHQR, where Q is —N(R)₂, and n is selected from 3, 4, and 5.

In certain embodiments, the disclosure provides a compound having the Formula (I), wherein R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5.

In certain embodiments, the disclosure provides a compound having the Formula (I), wherein R₂ and R₃ are independently selected from the group consisting of C₂₋₁₄ alkyl, C₂₋₁₄ alkenyl, —R*YR″, —YR″, and —R*OR″, or R₂ and R₃, together with the atom to which they are attached, form a heterocycle or carbocycle, and R₄ is —(CH₂)ₙQ or —(CH₂)ₙCHQR, where Q is —N(R)₂, and n is selected from 3, 4, and 5.

In certain embodiments, R₂ and R₃ are independently selected from the group consisting of C₂₋₁₄ alkyl, C₂₋₁₄ alkenyl, —R*YR″, —YR″, and —R*OR″, or R₂ and R₃, together with the atom to which they are attached, form a heterocycle or carbocycle.

In some embodiments, R₁ is selected from the group consisting of C₅₋₂₀ alkyl and C₅₋₂₀ alkenyl.

In other embodiments, R₁ is selected from the group consisting of —R*YR″, —YR″, and —R″M′R′.

In certain embodiments, R₁ is selected from —R*YR″ and —YR″. In some embodiments, Y is a cyclopropyl group. In some embodiments, R* is C₈ alkyl or C₈ alkenyl. In certain embodiments, R″ is C₃₋₁₂ alkyl. For example, R″ can be C₃ alkyl. For example, R″ can be C₄₋₈ alkyl (e.g., C₄, C₅, C₆, C₇, or C₈ alkyl).

In some embodiments, R₁ is C₅₋₂₀ alkyl. In some embodiments, R₁ is C₆ alkyl. In some embodiments, R₁ is C₈ alkyl. In other embodiments, R₁ is C₉ alkyl. In certain embodiments, R₁ is C₁₄ alkyl. In other embodiments, R₁ is C₁₈ alkyl.

In some embodiments, R₁ is C₅₋₂₀ alkenyl. In certain embodiments, R₁ is C₁₈ alkenyl. In some embodiments, R₁ is linoleyl.

In certain embodiments, R₁ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl, or heptadeca-9-yl). In certain embodiments, R₁ is

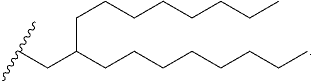 [SMILES: *CC(CCCCCCCC)CCCCCCCC]

In certain embodiments, R₁ is unsubstituted C₅₋₂₀ alkyl or C₅₋₂₀ alkenyl. In certain embodiments, R′ is substituted C₅₋₂₀ alkyl or C₅₋₂₀ alkenyl (e.g., substituted with a C₃₋₆ carbocycle such as 1-cyclopropylnonyl).

In other embodiments, R₁ is —R″M′R′.

In some embodiments, R′ is selected from —R*YR″ and —YR″. In some embodiments, Y is C₃₋₈ cycloalkyl. In some embodiments, Y is C₆₋₁₀ aryl. In some embodiments, Y is a cyclopropyl group. In some embodiments, Y is a cyclohexyl group.

In certain embodiments, R* is C₁ alkyl.

In some embodiments, R″ is selected from the group consisting of C₃₋₁₂ alkyl and C₃₋₁₂ alkenyl. In some embodiments, R″ adjacent to Y is C₁ alkyl. In some embodiments, R″ adjacent to Y is C₄₋₉ alkyl (e.g., C₄, C₅, C₆, C₇ or C₈ or C₉ alkyl).

In some embodiments, R′ is selected from C₄ alkyl and C₄ alkenyl. In certain embodiments, R′ is selected from C₅ alkyl and C₅ alkenyl. In some embodiments, R′ is selected from C₆ alkyl and C₆ alkenyl. In some embodiments, R′ is selected from C₇ alkyl and C₇ alkenyl.

In some embodiments, R′ is selected from C₉ alkyl and C₉ alkenyl.

In other embodiments, R′ is selected from C₁₁ alkyl and C₁₁ alkenyl. In other embodiments, R′ is selected from C₁₂ alkyl, C₁₂ alkenyl, C₁₃ alkyl, C₁₃ alkenyl, C₁₄ alkyl, C₁₄ alkenyl, C₁₅ alkyl, C₁₅ alkenyl, C₁₆ alkyl, C₁₆ alkenyl, C₁₇ alkyl, C₁₇ alkenyl, C₁₈ alkyl, and C₁₈ alkenyl. In certain embodiments, R′ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl or heptadeca-9-yl).

In certain embodiments, R′ is

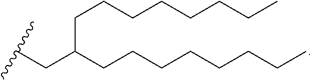 [SMILES: *CC(CCCCCCCC)CCCCCCCC]

In certain embodiments, R′ is unsubstituted C₁₋₁₈ alkyl. In certain embodiments, R′ is substituted C₁₋₁₈ alkyl (e.g., C₁₋₁₅ alkyl substituted with a C₃₋₆ carbocycle such as 1-cyclopropylnonyl).

In some embodiments, R″ is selected from the group consisting of C₃₋₁₄ alkyl and C₃₋₁₄ alkenyl. In some embodiments, R″ is C₃ alkyl, C₄ alkyl, C₅ alkyl, C₆ alkyl, C₇ alkyl, or C₈ alkyl. In some embodiments, R″ is C₉ alkyl, C₁₀ alkyl, C₁₁ alkyl, C₁₂ alkyl, C₁₃ alkyl, or C₁₄ alkyl.

In some embodiments, M′ is —C(O)O—. In some embodiments, M′ is —OC(O)—.

In other embodiments, M′ is an aryl group or heteroaryl group. For example, M′ can be selected from the group consisting of phenyl, oxazole, and thiazole.

In some embodiments, M is —C(O)O—. In some embodiments, M is —OC(O)—. In some embodiments, M is —C(O)N(R′)—. In some embodiments, M is —P(O)(OR′)O—.

In other embodiments, M is an aryl group or heteroaryl group. For example, M can be selected from the group consisting of phenyl, oxazole, and thiazole.

In some embodiments, M is the same as M′. In other embodiments, M is different from M′.

In some embodiments, each R₅ is H. In certain such embodiments, each R₆ is also H.

In some embodiments, R₇ is H. In other embodiments, R₇ is C₁₋₃ alkyl (e.g., methyl, ethyl, propyl, or i-propyl).

In some embodiments, R₂ and R₃ are independently C₅₋₁₄ alkyl or C₅₋₁₄ alkenyl.

In some embodiments, R₂ and R₃ are the same. In some embodiments, R₂ and R₃ are C₈ alkyl. In certain embodiments, R₂ and R₃ are C₂ alkyl. In other embodiments, R₂ and R₃ are C₃ alkyl. In some embodiments, R₂ and R₃ are C₄ alkyl. In certain embodiments, R₂ and R₃ are C₅ alkyl. In other embodiments, R₂ and R₃ are C₆ alkyl. In some embodiments, R₂ and R₃ are C₇ alkyl.

In other embodiments, R₂ and R₃ are different. In certain embodiments, R₂ is C₈ alkyl. In some embodiments, R₃ is C₁_7 (e.g., C₁, C₂, C₃, C₄, C₅, C₆, or C₇ alkyl) or C₉ alkyl.

In some embodiments, R₇ and R₃ are H.

In certain embodiments, R₂ is H.

In some embodiments, m is 5, 7, or 9.

In some embodiments, R₄ is selected from —(CH₂)ₙQ and —(CH₂)ₙCHQR.

In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), —C(R)N(R)₂C(O)OR, a carbocycle, and a heterocycle.

In certain embodiments, Q is —OH.

In certain embodiments, Q is a substituted or unsubstituted 5- to 10-membered heteroaryl, e.g., Q is an imidazole, a pyrimidine, a purine, 2-amino-1,9-dihydro-6H-purin-6-one-9-yl (or guanin-9-yl), adenin-9-yl, cytosin-1-yl, or uracil-1-yl. In certain embodiments, Q is a substituted 5- to 14-membered heterocycloalkyl, e.g., substituted with one or more substituents selected from oxo (═O), OH, amino, and C₁₋₃ alkyl. For example, Q is 4-methylpiperazinyl, 4-(4-methoxybenzyl)piperazinyl, or isoindolin-2-yl-1,3-dione.

In certain embodiments, Q is an unsubstituted or substituted C₆₋₁₀ aryl (such as phenyl) or C₃₋₆ cycloalkyl.

In some embodiments, n is 1. In other embodiments, n is 2. In further embodiments, n is 3. In certain other embodiments, n is 4. For example, R₄ can be —(CH₂)₂OH. For example, R₄ can be —(CH₂)₃OH. For example, R₄ can be —(CH₂)₄OH. For example, R₄ can be benzyl. For example, R₄ can be 4-methoxybenzyl.

In some embodiments, R₄ is a C₃₋₆ carbocycle. In some embodiments, R₄ is a C₃₋₆ cycloalkyl. For example, R₄ can be cyclohexyl optionally substituted with e.g., OH, halo, C₁₋₆ alkyl, etc. For example, R₄ can be 2-hydroxycyclohexyl.

In some embodiments, R is H.

In some embodiments, R is unsubstituted C₁₋₃ alkyl or unsubstituted C₂₋₃ alkenyl. For example, R₄ can be —CH₂CH(OH)CH₃ or —CH₂CH(OH)CH₂CH₃.

In some embodiments, R is substituted C₁₋₃ alkyl, e.g., CH₂OH. For example, R₄ can be —CH₂CH(OH)CH₂OH.

In some embodiments, R₂ and R₃, together with the atom to which they are attached, form a heterocycle or carbocycle. In some embodiments, R₂ and R₃, together with the atom to which they are attached, form a 5- to 14-membered aromatic or non-aromatic heterocycle having one or more heteroatoms selected from N, O, S, and P. In some embodiments, R₂ and R₃, together with the atom to which they are attached, form an optionally substituted C₃₋₂₀ carbocycle (e.g., C₃₋₁₈ carbocycle, C₃₋₁₅ carbocycle, C₃₋₁₂ carbocycle, or C₃₋₁₀ carbocycle), either aromatic or non-aromatic. In some embodiments, R₂ and R₃, together with the atom to which they are attached, form a C₃₋₆ carbocycle. In other embodiments, R₂ and R₃, together with the atom to which they are attached, form a C₆ carbocycle, such as a cyclohexyl or phenyl group. In certain embodiments, the heterocycle or C₃₋₆ carbocycle is substituted with one or more alkyl groups (e.g., at the same ring atom or at adjacent or non-adjacent ring atoms). For example, R₂ and R₃, together with the atom to which they are attached, can form a cyclohexyl or phenyl group bearing one or more C₅ alkyl substitutions. In certain embodiments, the heterocycle or C₃₋₆ carbocycle formed by R₂ and R₃, is substituted with a carbocycle groups. For example, R₂ and R₃, together with the atom to which they are attached, can form a cyclohexyl or phenyl group that is substituted with cyclohexyl. In some embodiments, R₂ and R₃, together with the atom to which they are attached, form a C₇₋₁₅ carbocycle, such as a cycloheptyl, cyclopentadecanyl, or naphthyl group.

In some embodiments, R₄ is selected from —(CH₂)ₙQ and —(CH₂)ₙCHQR. In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle. In other embodiments, Q is selected from the group consisting of an imidazole, a pyrimidine, and a purine.

In some embodiments, R₂ and R₃, together with the atom to which they are attached, form a heterocycle or carbocycle. In some embodiments, R₂ and R₃, together with the atom to which they are attached, form a C₃₋₆ carbocycle, such as a phenyl group. In certain embodiments, the heterocycle or C₃₋₆ carbocycle is substituted with one or more alkyl groups (e.g., at the same ring atom or at adjacent or non-adjacent ring atoms). For example, R₂ and R₃, together with the atom to which they are attached, can form a phenyl group bearing one or more C₅ alkyl substitutions.

In some embodiments, the pharmaceutical compositions of the present disclosure, the compound of formula (I) is selected from the group consisting of:

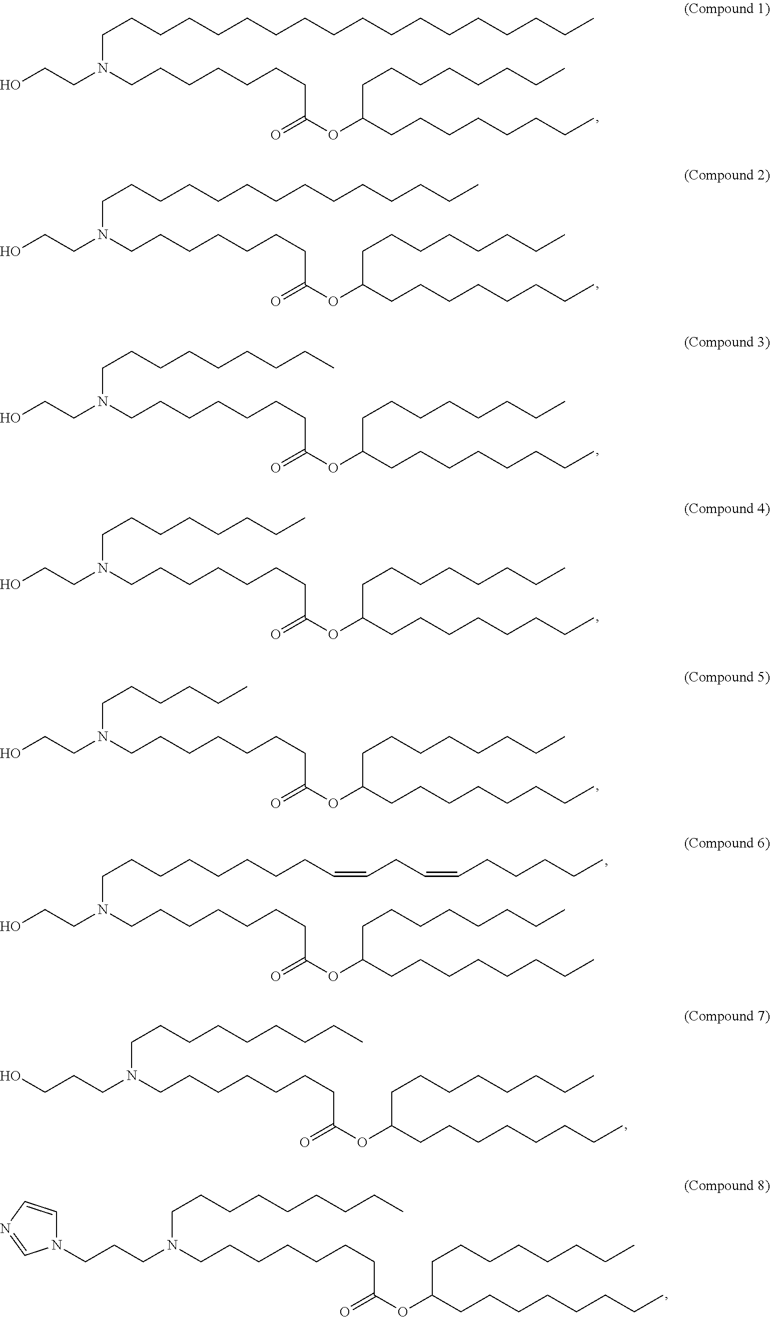

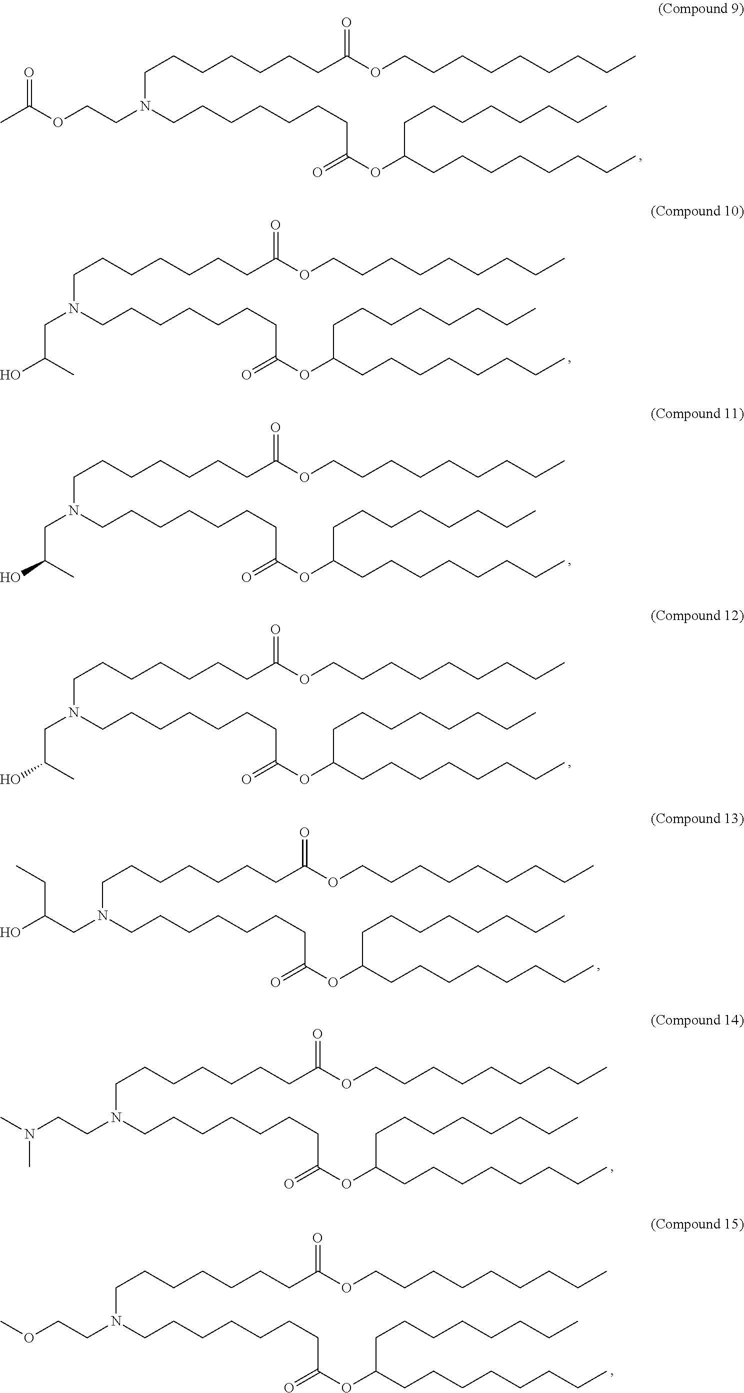

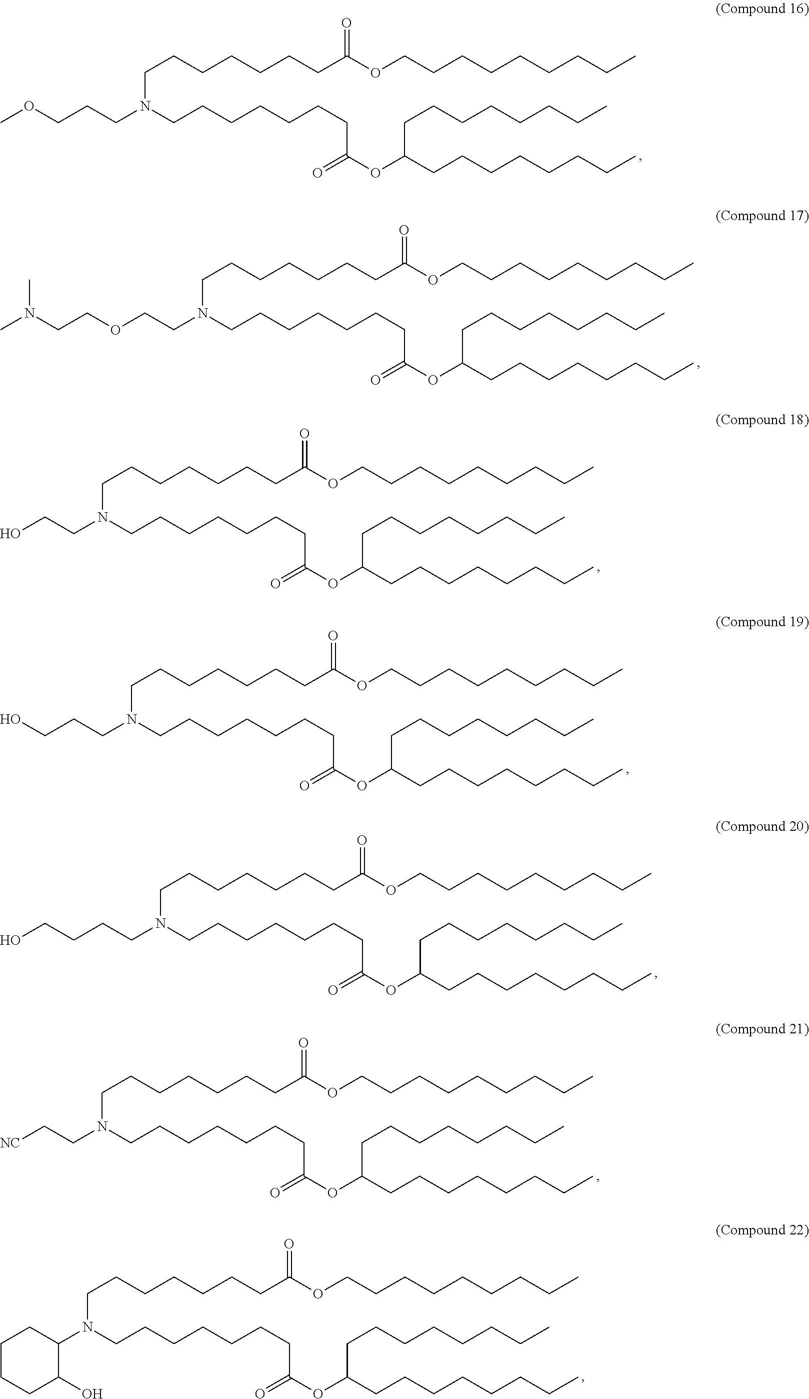

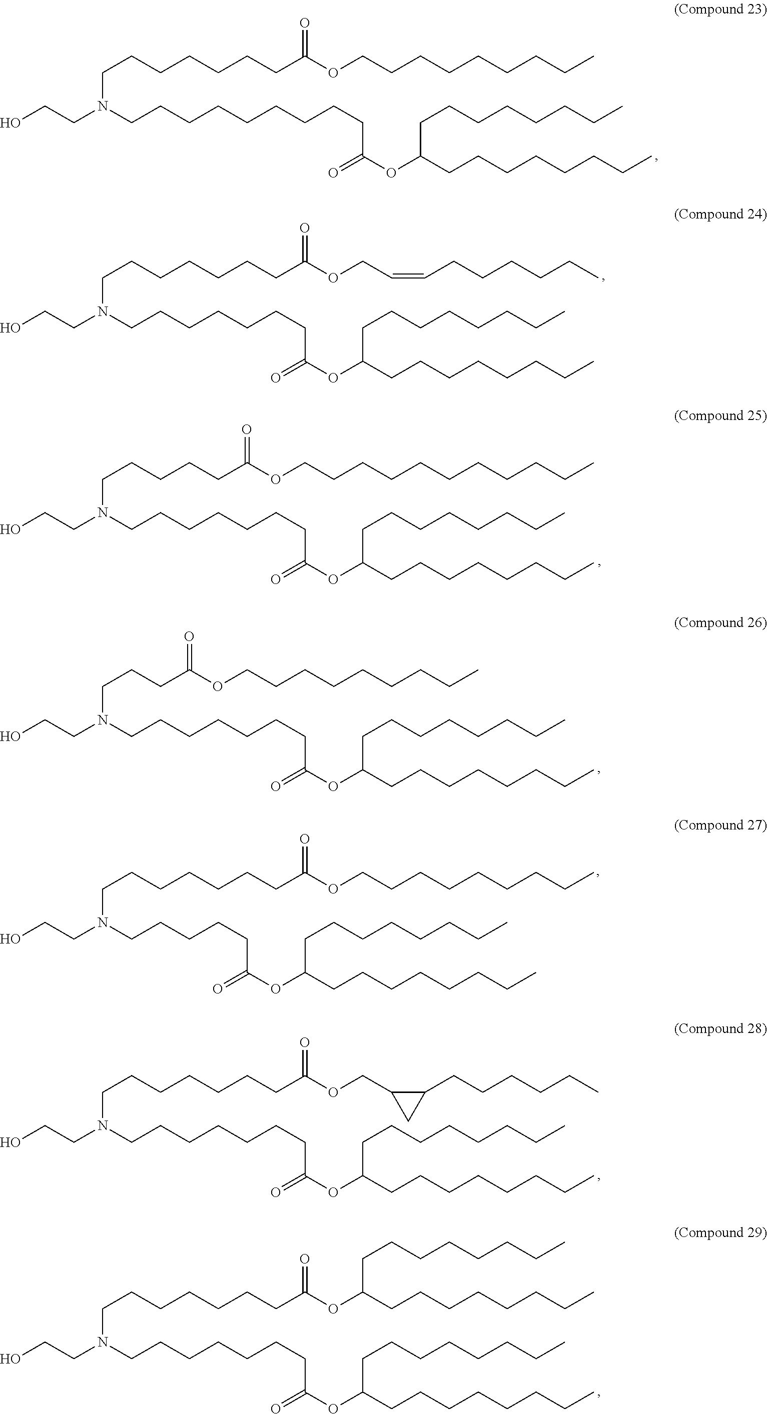

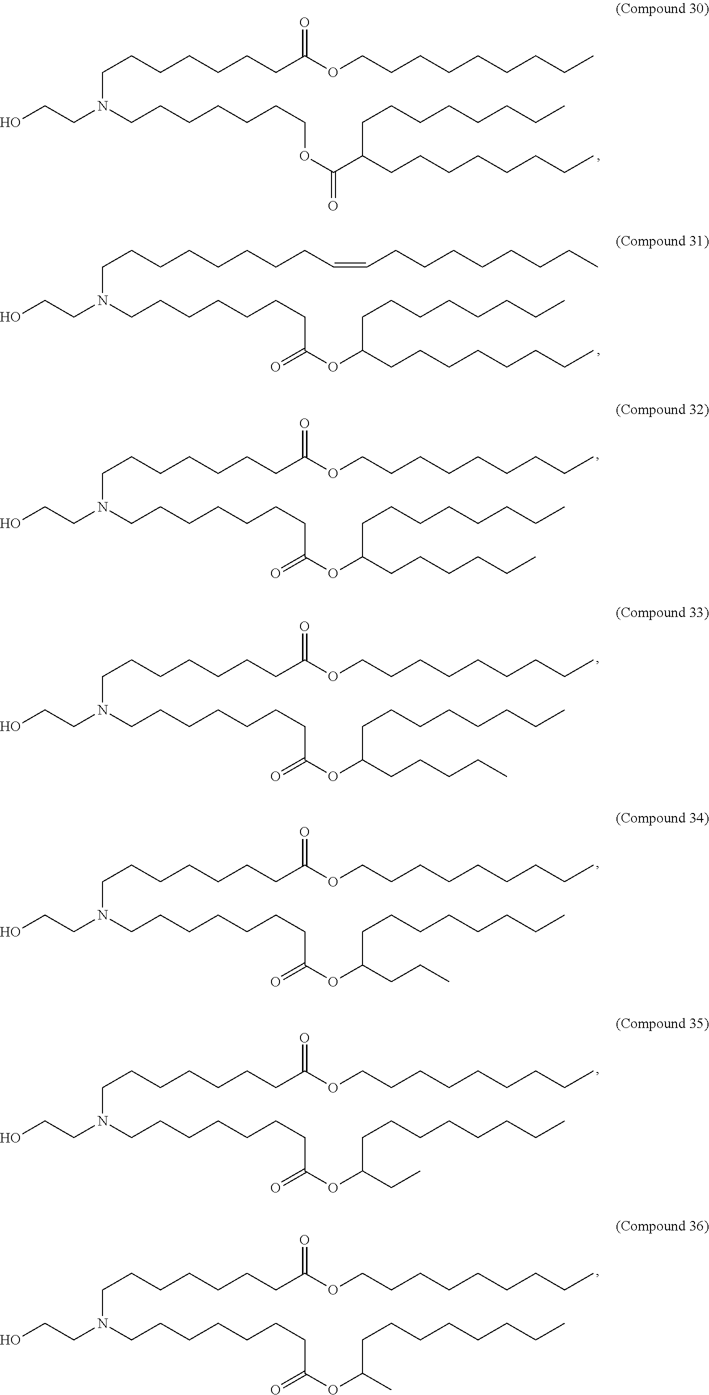

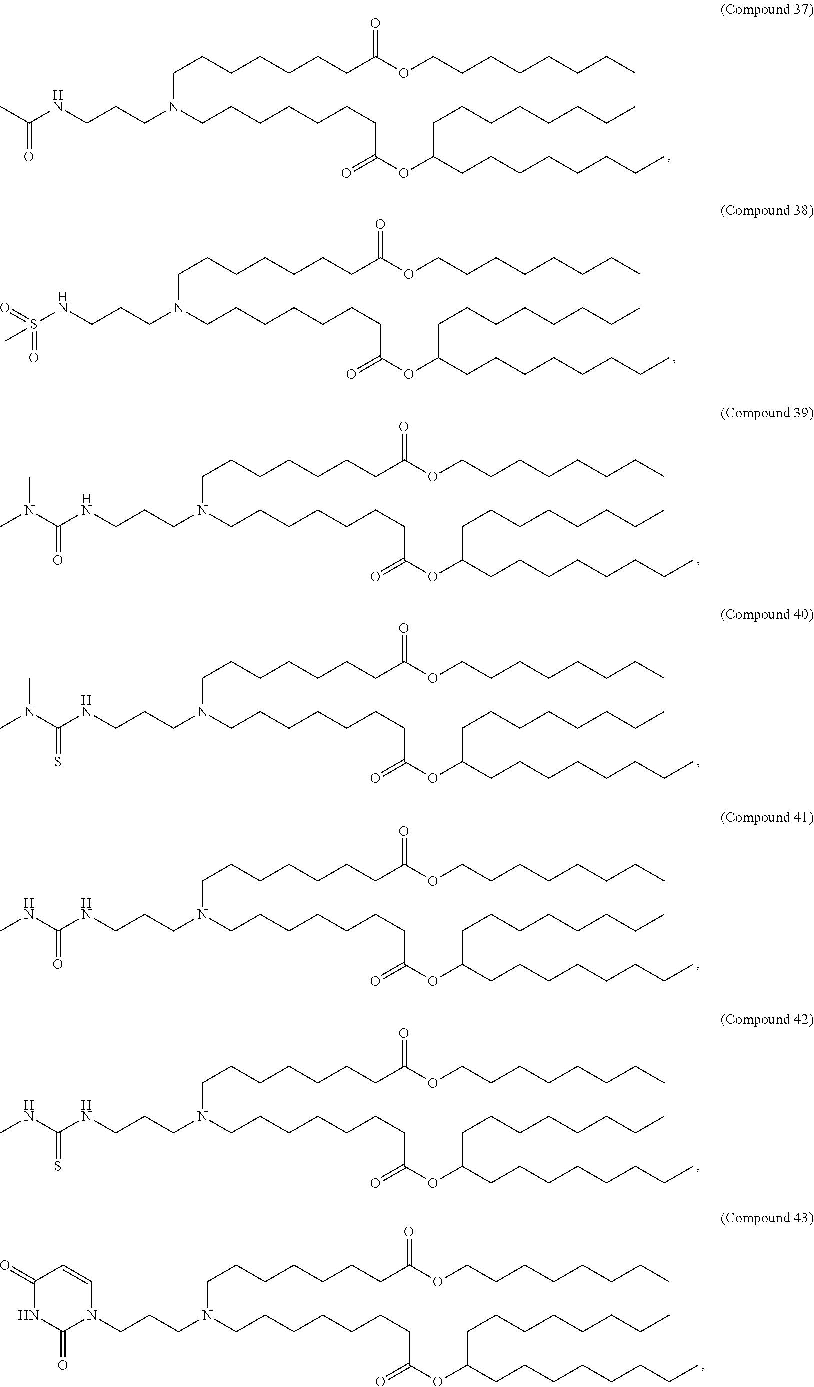

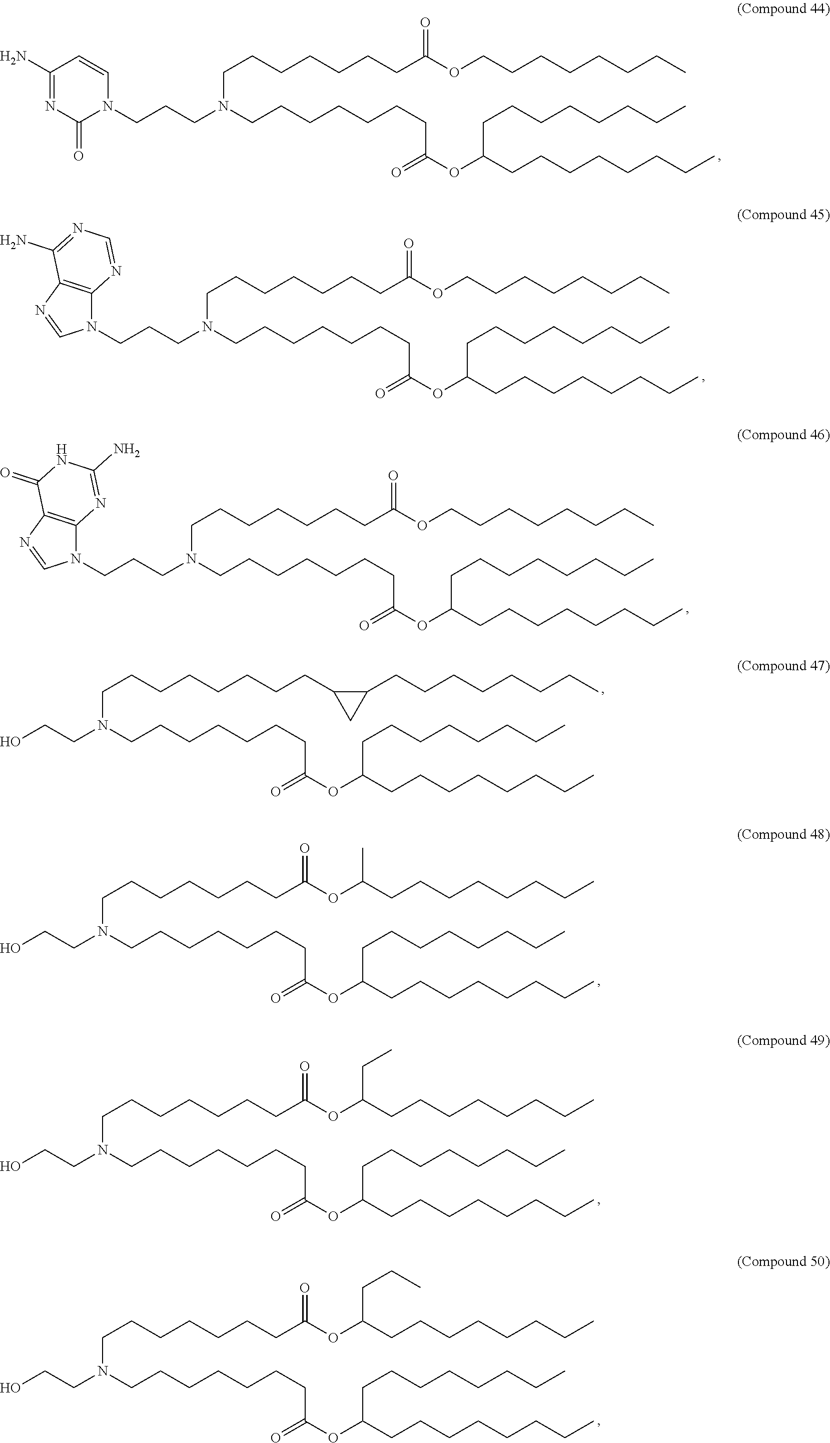

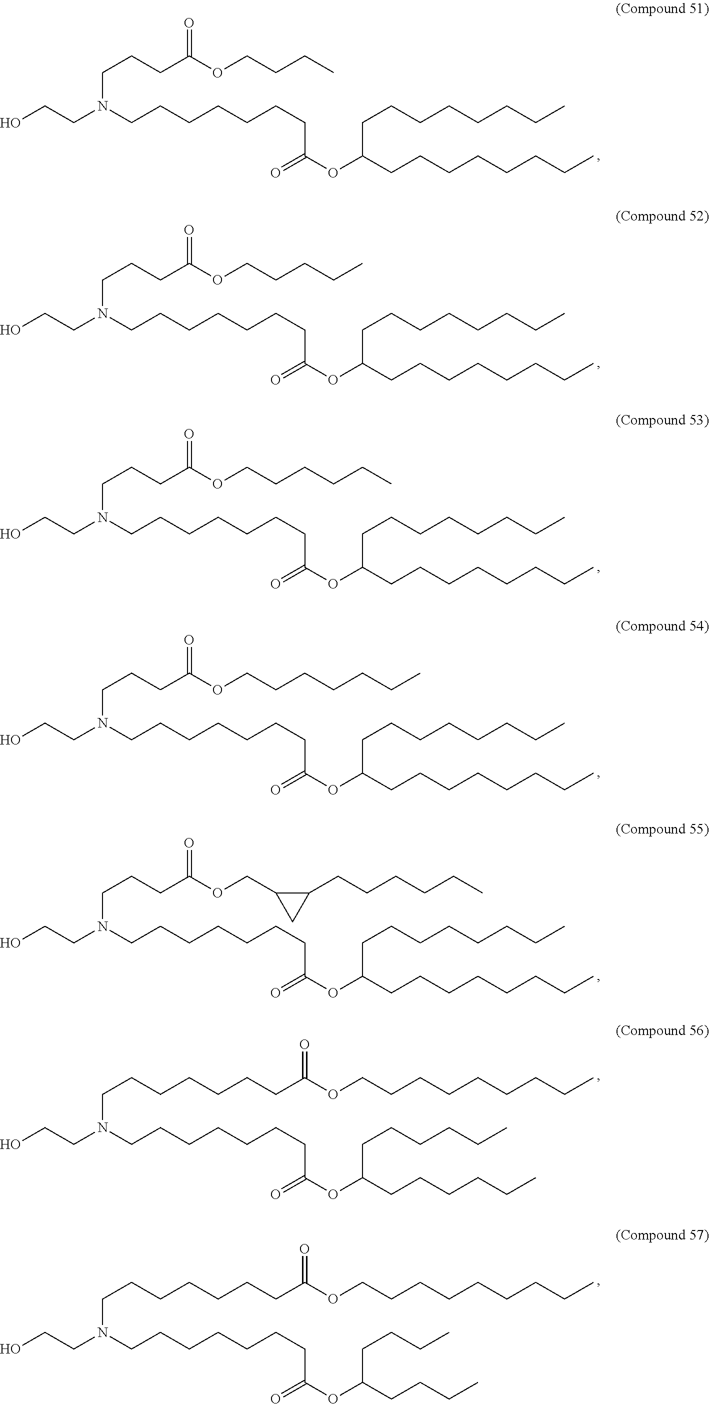

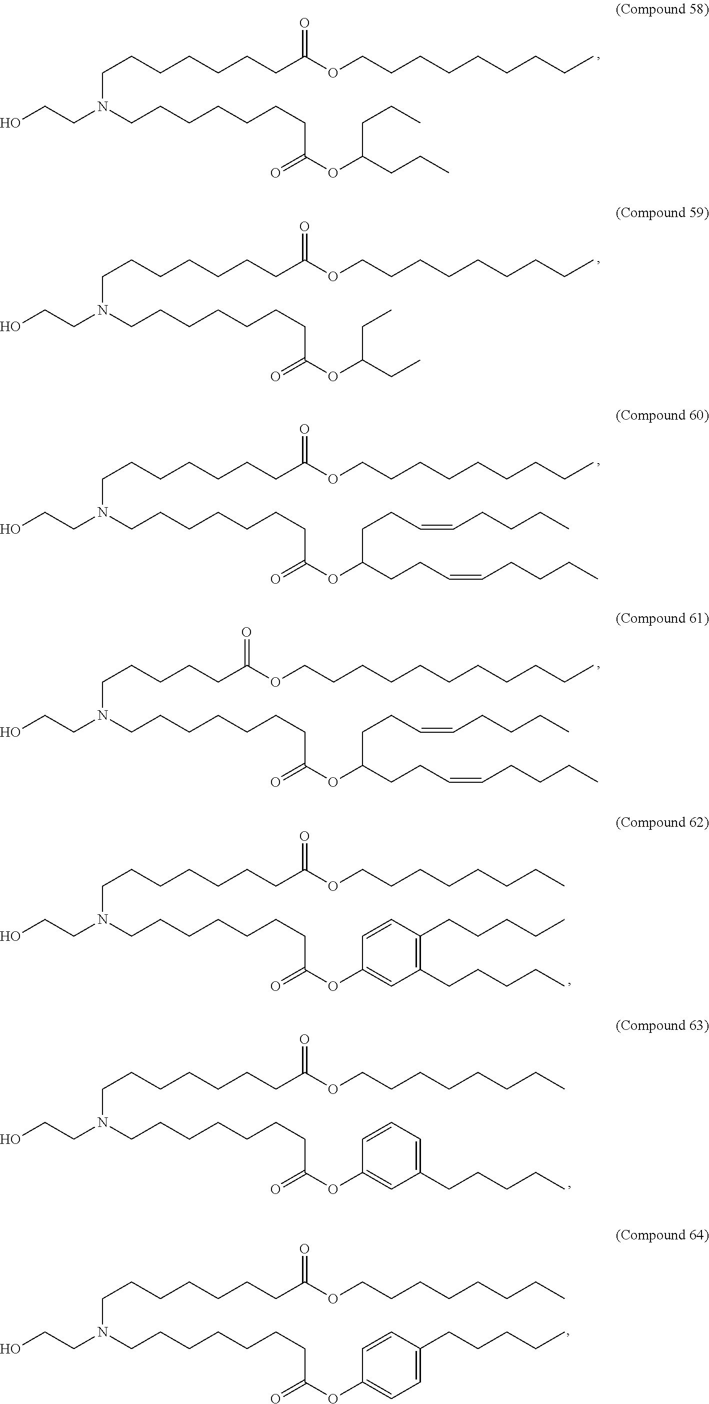

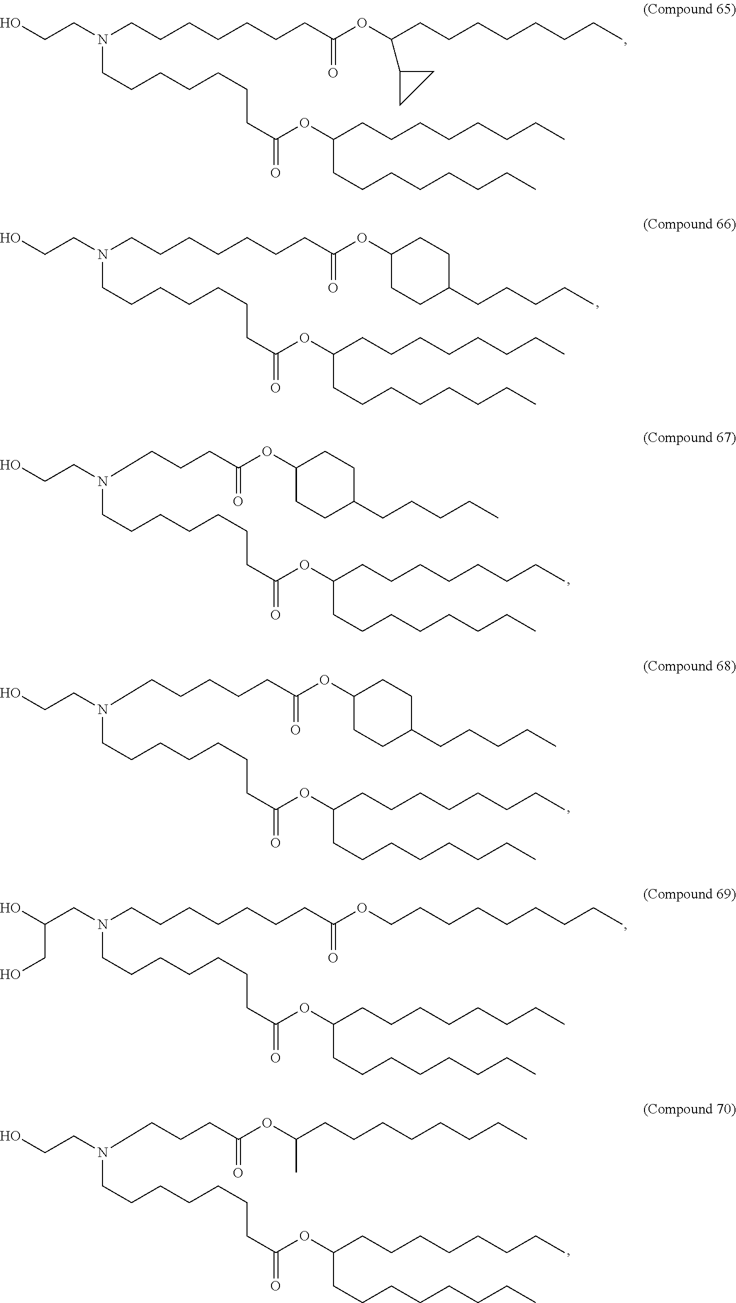 [SMILES: CCCCCCCCC(CCCCCCCC)OC(=O)CCCCCCCN(CCO)CCCCCCCC(=O)OC(CCCCCCCC)C1CC1 ; CCCCCCCCC(CCCCCCCC)OC(=O)CCCCCCCN(CCO)CCCCCCCC(=O)OC1CCC(CCCCC)CC1 ; CCCCCCCCC(CCCCCCCC)OC(=O)CCCCCCCN(CCO)CCCC(=O)OC1CCC(CCCCC)CC1 ; CCCCCCCCC(CCCCCCCC)OC(=O)CCCCCCCN(CCO)CCCCCC(=O)OC1CCC(CCCCC)CC1 ; CCCCCCCCCOC(=O)CCCCCCCN(CCCCCCCC(=O)OC(CCCCCCCC)CCCCCCCC)CC(O)CO ; CCCCCCCCC(C)OC(=O)CCCN(CCO)CCCCCCCC(=O)OC(CCCCCCCC)CCCCCCCC]

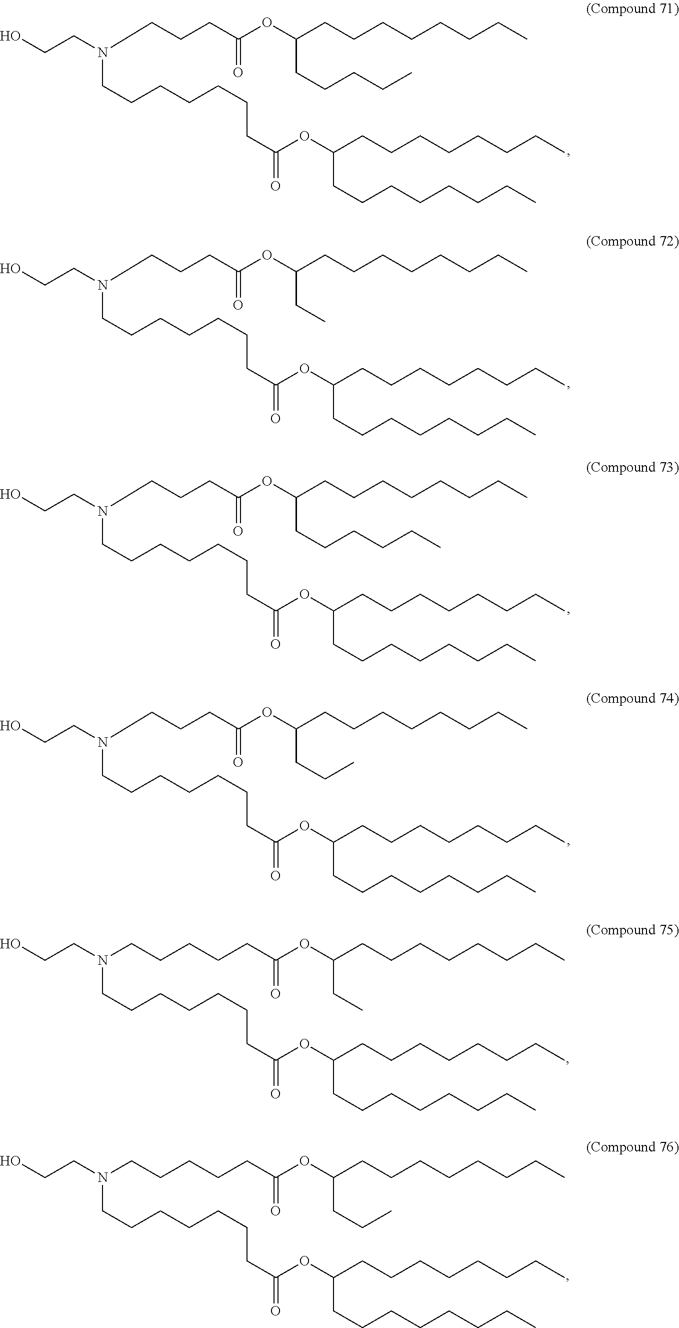 [SMILES: CCCCCCCCC(CCCCCCCC)OC(=O)CCCCCCCN(CCO)CCCC(=O)OC(CCCCC)CCCCCCCC ; CCCCCCCCC(CC)OC(=O)CCCN(CCO)CCCCCCCC(=O)OC(CCCCCCCC)CCCCCCCC ; CCCCCCCCC(CCCCCCCC)OC(=O)CCCCCCCN(CCO)CCCC(=O)OC(CCCCCC)CCCCCCCC ; CCCCCCCCC(CCC)OC(=O)CCCN(CCO)CCCCCCCC(=O)OC(CCCCCCCC)CCCCCCCC ; CCCCCCCCC(CC)OC(=O)CCCCCN(CCO)CCCCCCCC(=O)OC(CCCCCCCC)CCCCCCCC ; CCCCCCCCC(CCC)OC(=O)CCCCCN(CCO)CCCCCCCC(=O)OC(CCCCCCCC)CCCCCCCC]

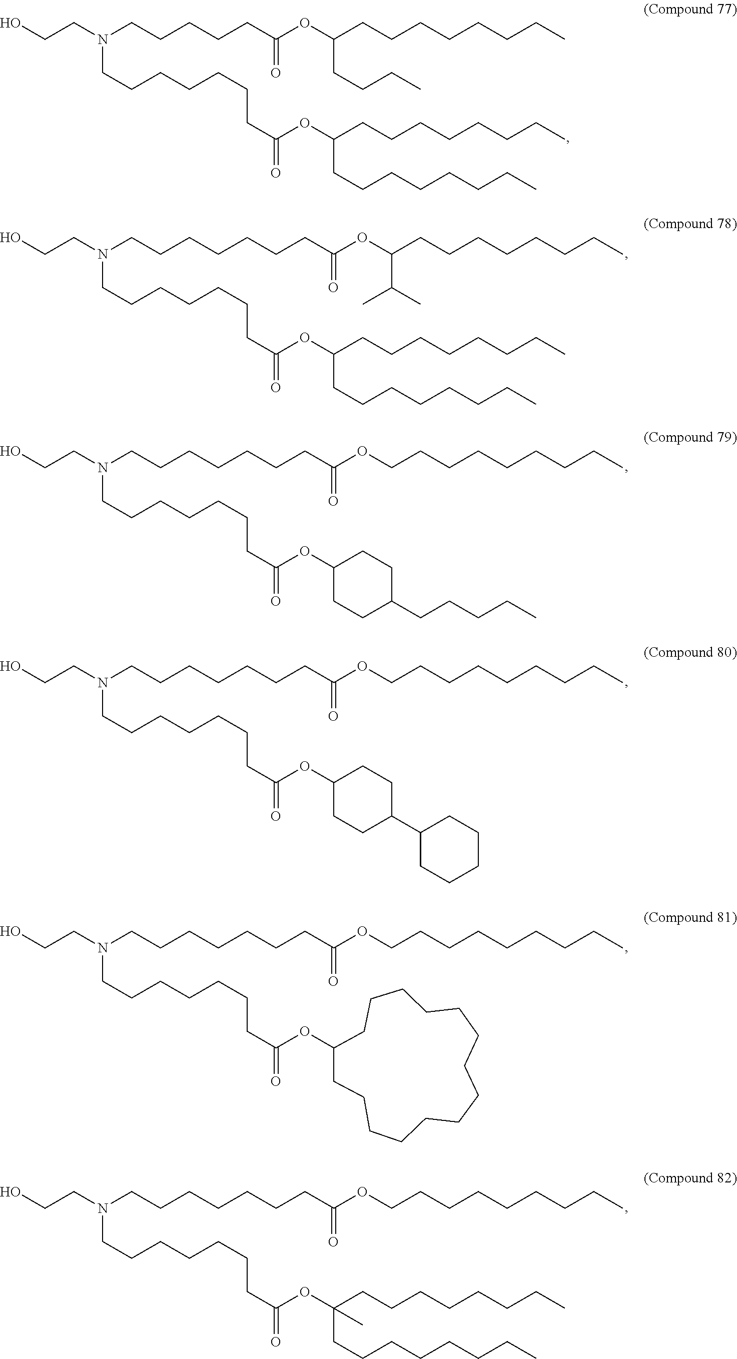 [SMILES: CCCCCCCCC(CCCC)OC(=O)CCCCCN(CCO)CCCCCCCC(=O)OC(CCCCCCCC)CCCCCCCC ; CCCCCCCCC(CCCCCCCC)OC(=O)CCCCCCCN(CCO)CCCCCCCC(=O)OC(CCCCCCCC)C(C)C ; CCCCCCCCCOC(=O)CCCCCCCN(CCO)CCCCCCCC(=O)OC1CCC(CCCCC)CC1 ; CCCCCCCCCOC(=O)CCCCCCCN(CCO)CCCCCCCC(=O)OC1CCC(C2CCCCC2)CC1 ; CCCCCCCCCOC(=O)CCCCCCCN(CCO)CCCCCCCC(=O)OC1CCCCCCCCCCCCCC1 ; CCCCCCCCCOC(=O)CCCCCCCN(CCO)CCCCCCCC(=O)OC(C)(CCCCCCCC)CCCCCCCC]

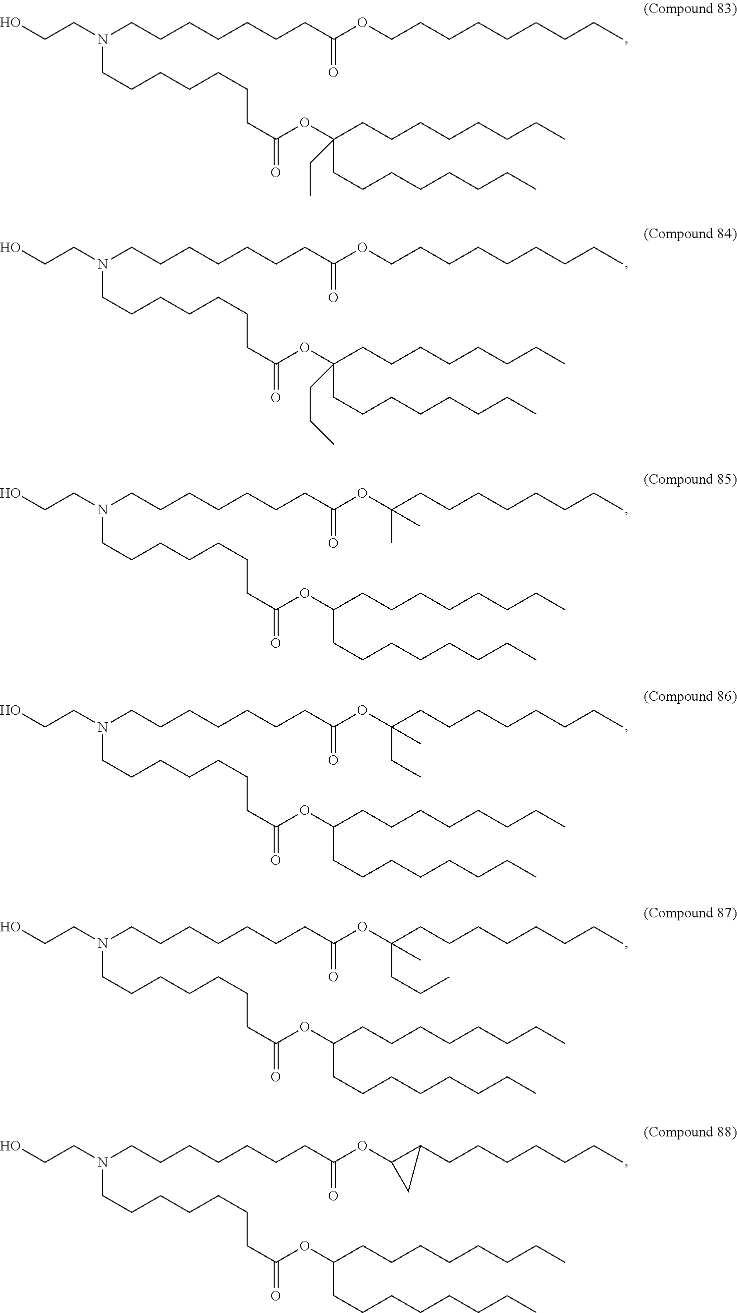 [SMILES: CCCCCCCCCOC(=O)CCCCCCCN(CCO)CCCCCCCC(=O)OC(CC)(CCCCCCCC)CCCCCCCC ; CCCCCCCCCOC(=O)CCCCCCCN(CCO)CCCCCCCC(=O)OC(CCC)(CCCCCCCC)CCCCCCCC ; CCCCCCCCC(CCCCCCCC)OC(=O)CCCCCCCN(CCO)CCCCCCCC(=O)OC(C)(C)CCCCCCCC ; CCCCCCCCC(CCCCCCCC)OC(=O)CCCCCCCN(CCO)CCCCCCCC(=O)OC(C)(CC)CCCCCCCC ; CCCCCCCCC(CCCCCCCC)OC(=O)CCCCCCCN(CCO)CCCCCCCC(=O)OC(C)(CCC)CCCCCCCC ; CCCCCCCCC(CCCCCCCC)OC(=O)CCCCCCCN(CCO)CCCCCCCC(=O)OC1CC1CCCCCCC]

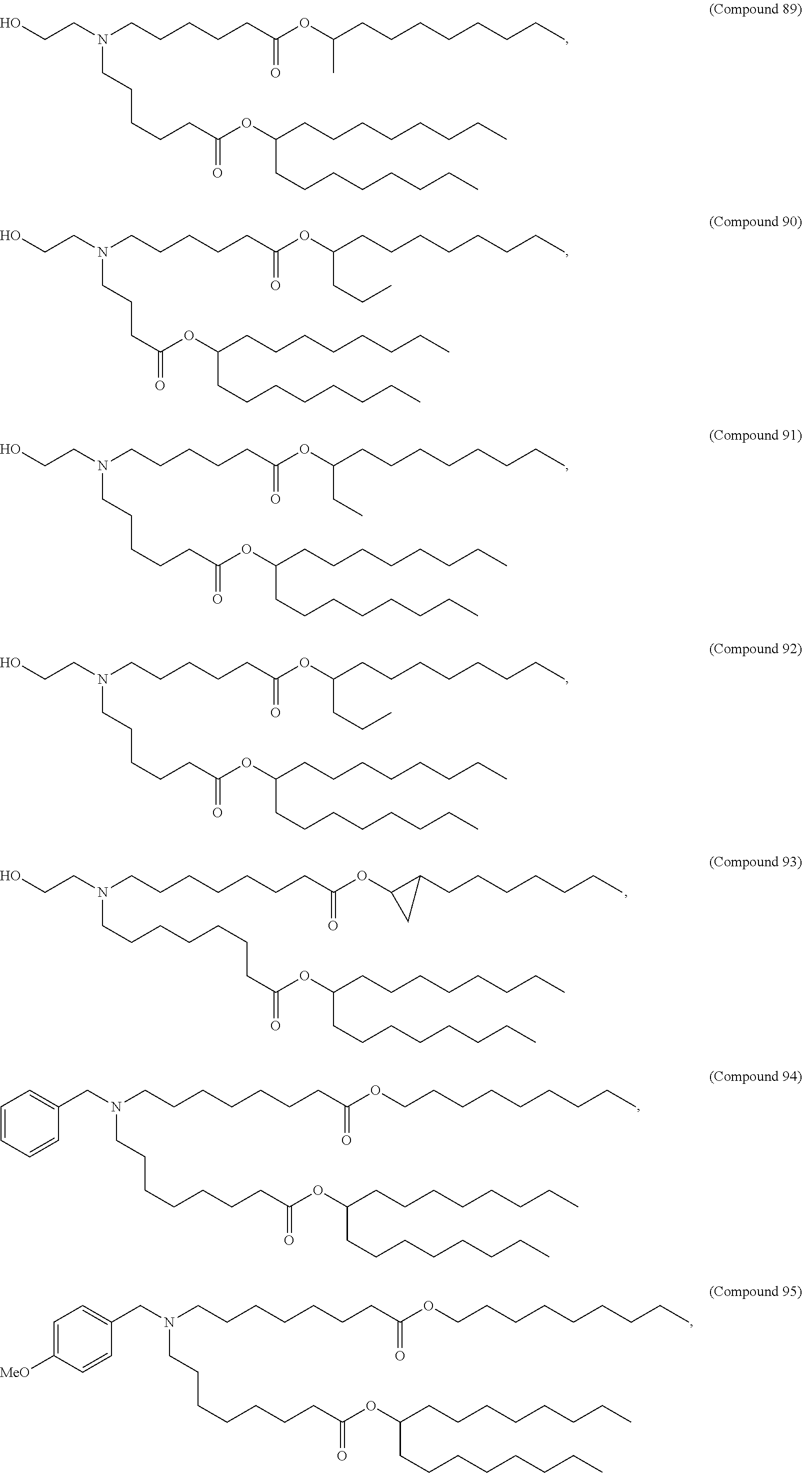

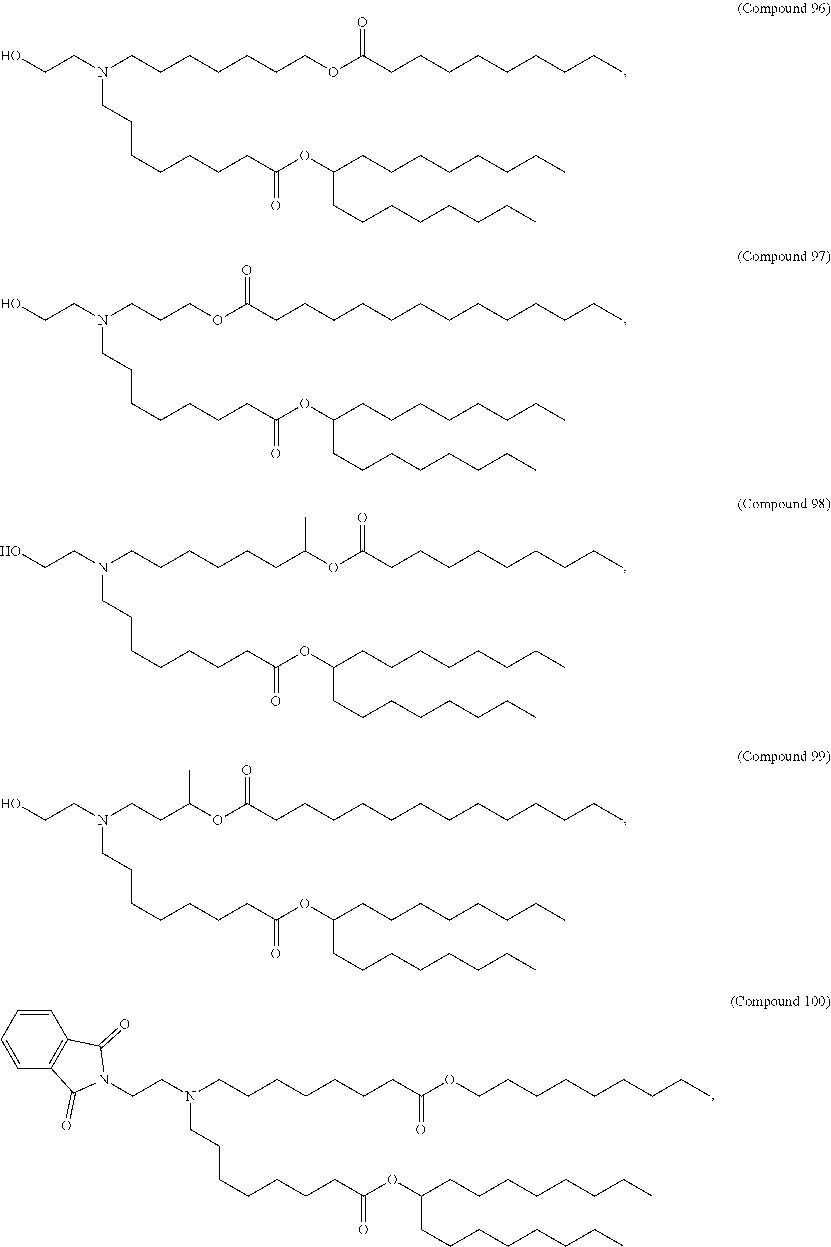 [SMILES: CCCCCCCCCC(=O)OCCCCCCCN(CCO)CCCCCCCC(=O)OC(CCCCCCCC)CCCCCCCC ; CCCCCCCCCCCCCC(=O)OCCCN(CCO)CCCCCCCC(=O)OC(CCCCCCCC)CCCCCCCC ; CCCCCCCCCC(=O)OC(C)CCCCCCN(CCO)CCCCCCCC(=O)OC(CCCCCCCC)CCCCCCCC ; CCCCCCCCCCCCCC(=O)OC(C)CCN(CCO)CCCCCCCC(=O)OC(CCCCCCCC)CCCCCCCC ; CCCCCCCCCOC(=O)CCCCCCCN(CCCCCCCC(=O)OC(CCCCCCCC)CCCCCCCC)CCN1C(=O)c2ccccc2C1=O]

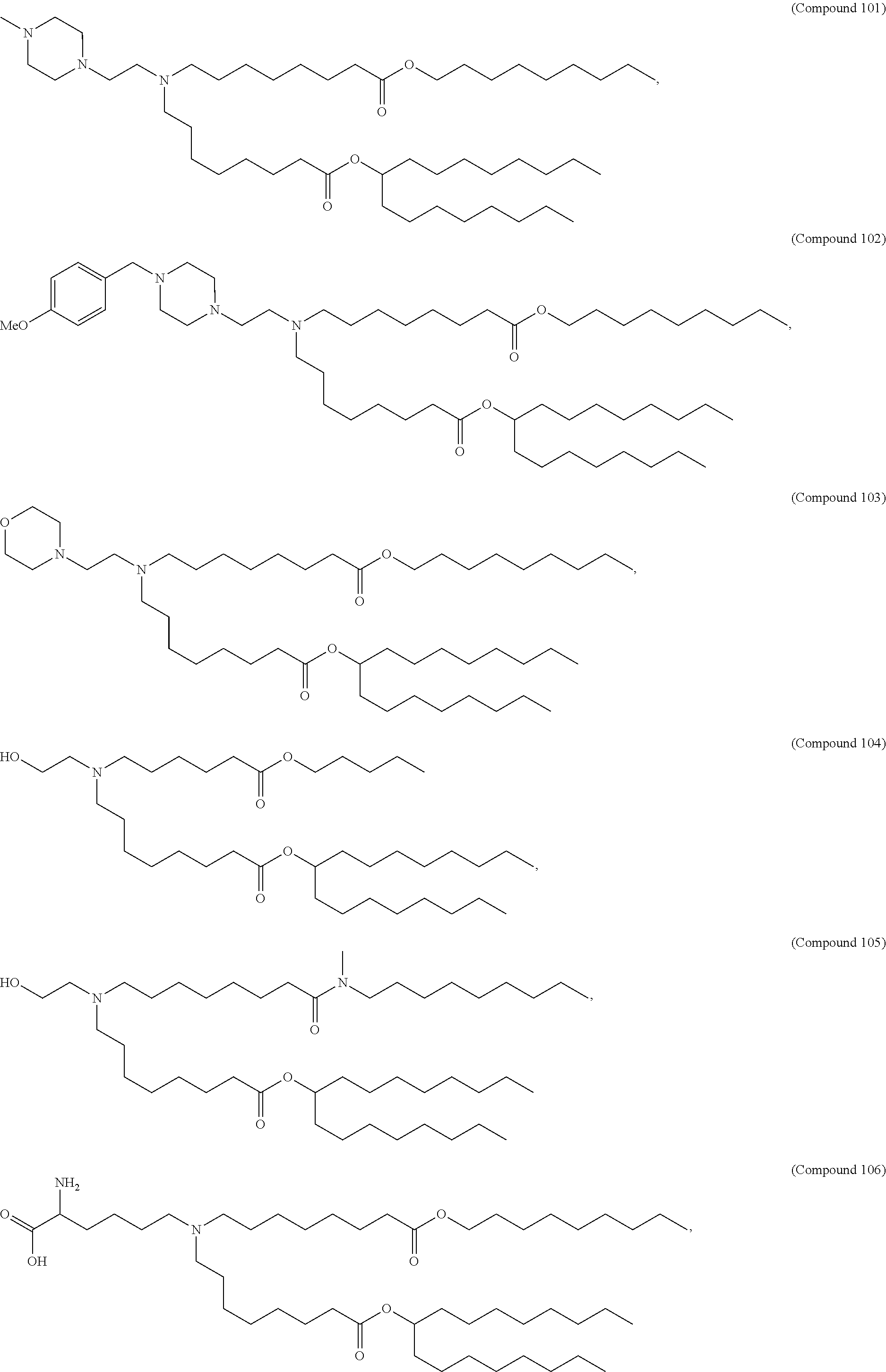

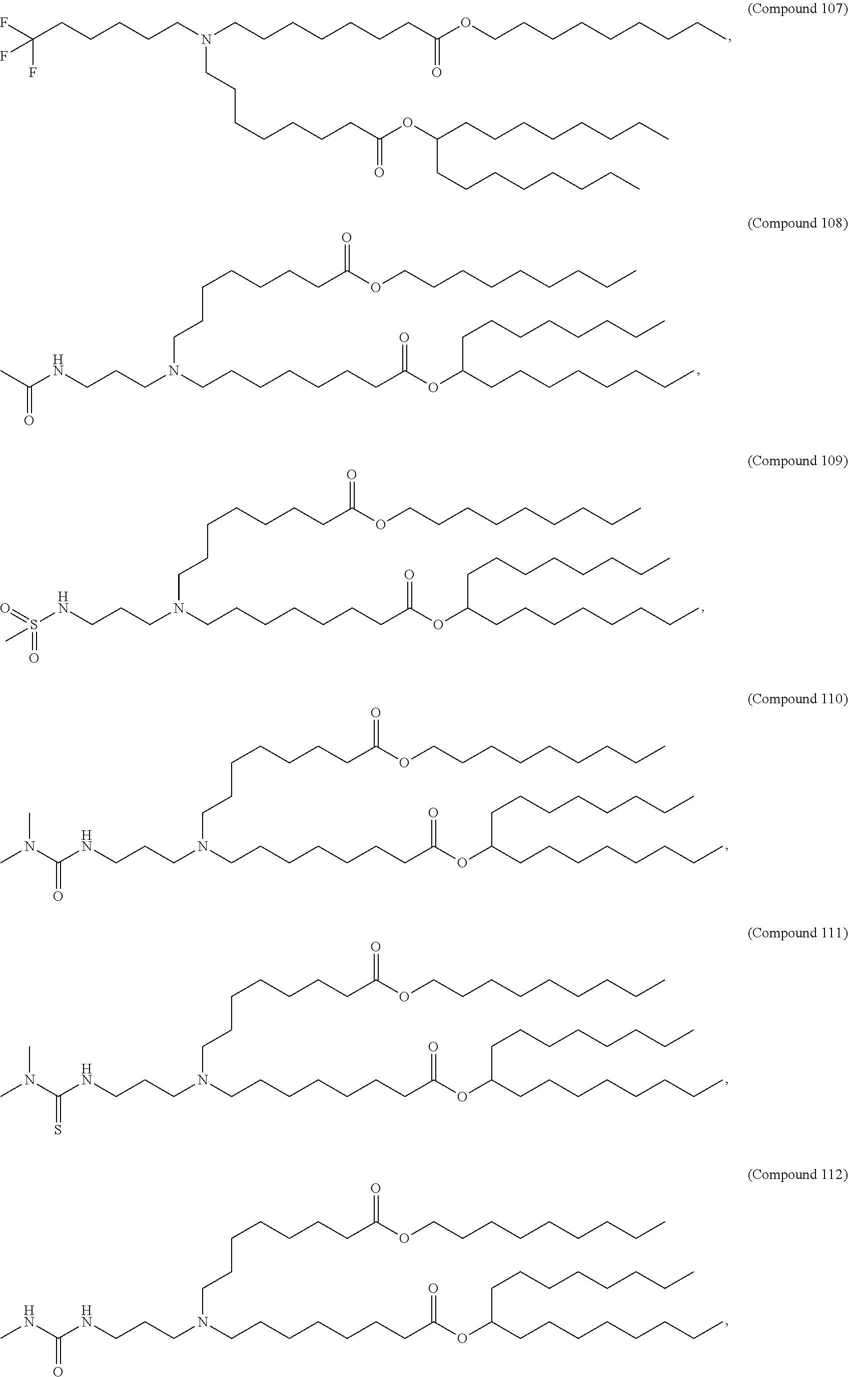

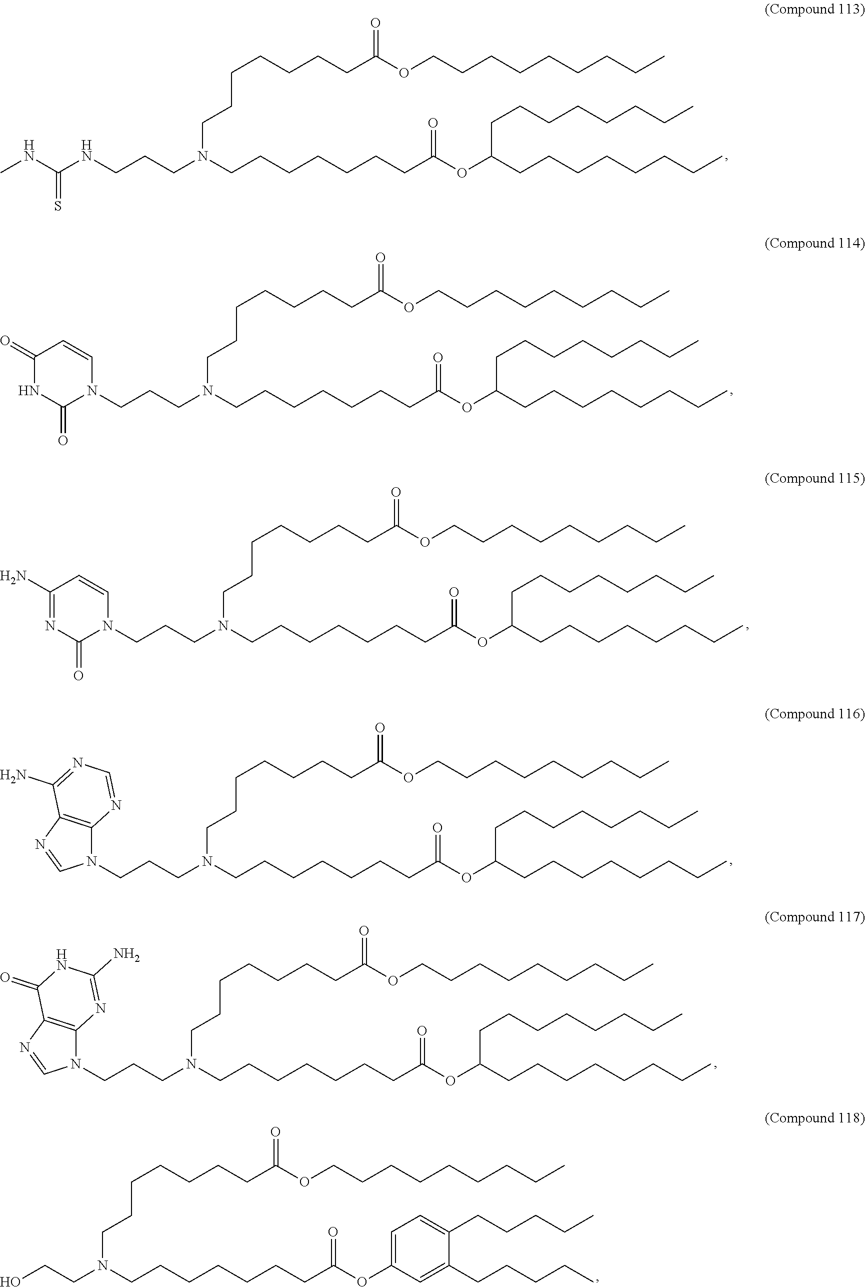 [SMILES: CCCCCCCCCOC(=O)CCCCCCCN(CCCCCCCC(=O)OC(CCCCCCCC)CCCCCCCC)CCCNC(=S)NC ; CCCCCCCCCOC(=O)CCCCCCCN(CCCCCCCC(=O)OC(CCCCCCCC)CCCCCCCC)CCCn1ccc(=O)[nH]c1=O ; CCCCCCCCCOC(=O)CCCCCCCN(CCCCCCCC(=O)OC(CCCCCCCC)CCCCCCCC)CCCn1ccc(N)nc1=O ; CCCCCCCCCOC(=O)CCCCCCCN(CCCCCCCC(=O)OC(CCCCCCCC)CCCCCCCC)CCCn1cnc2c(N)ncnc21 ; CCCCCCCCCOC(=O)CCCCCCCN(CCCCCCCC(=O)OC(CCCCCCCC)CCCCCCCC)CCCn1cnc2c(=O)[nH]c(N)nc21 ; CCCCCCCCCOC(=O)CCCCCCCN(CCO)CCCCCCCC(=O)Oc1ccc(CCCCC)c(CCCCC)c1]

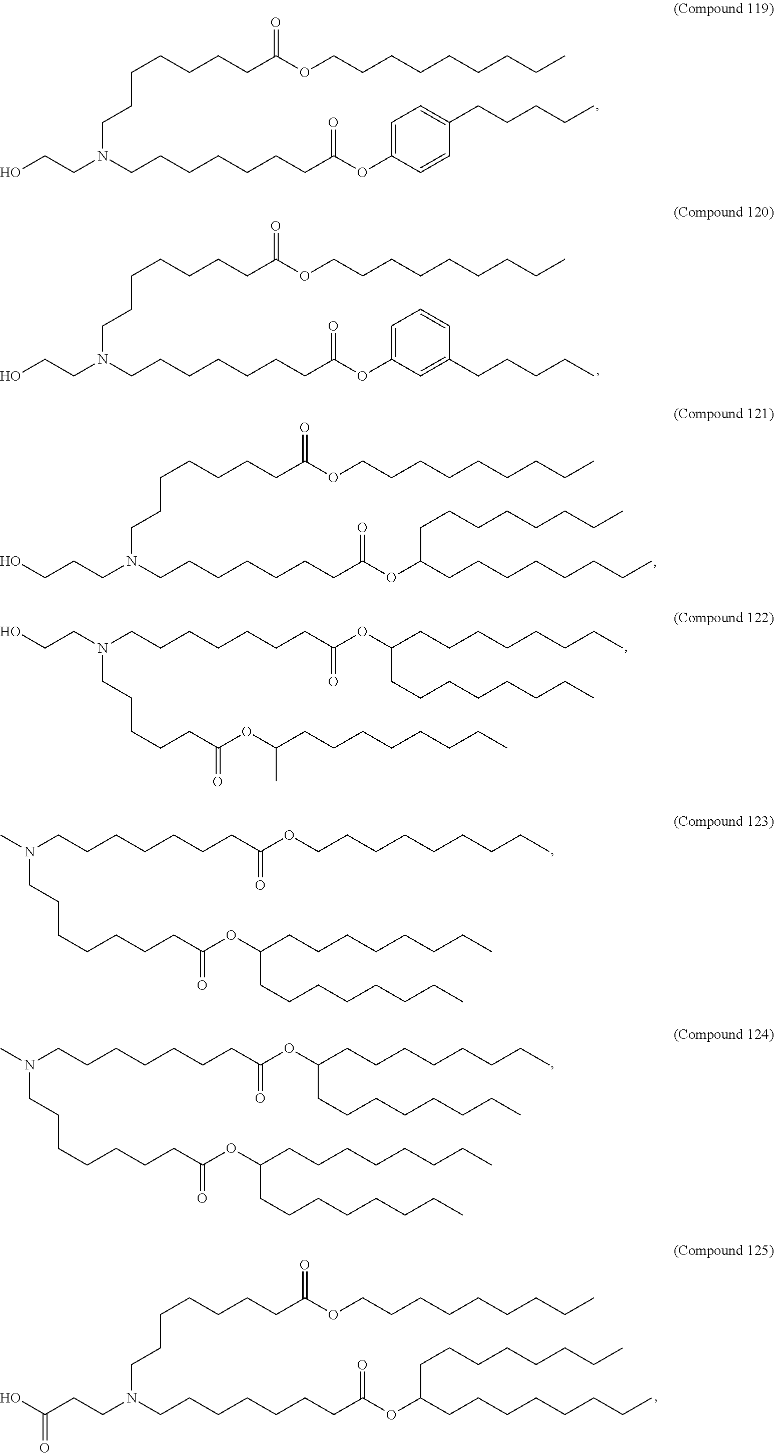

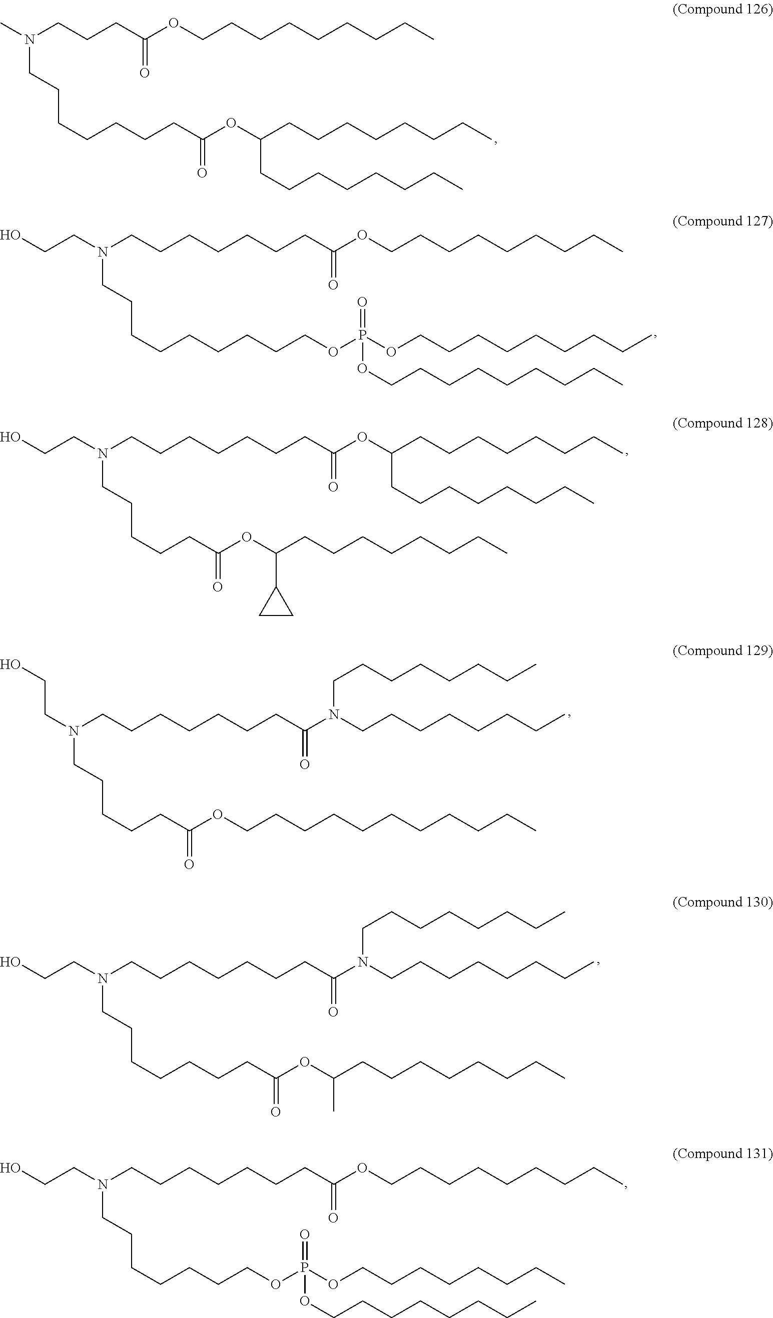 [SMILES: CCCCCCCCCOC(=O)CCCN(C)CCCCCCCC(=O)OC(CCCCCCCC)CCCCCCCC ; CCCCCCCCCOC(=O)CCCCCCCN(CCO)CCCCCCCCCOP(=O)(OCCCCCCCCC)OCCCCCCCCC ; CCCCCCCCC(CCCCCCCC)OC(=O)CCCCCCCN(CCO)CCCCCC(=O)OC(CCCCCCCC)C1CC1 ; CCCCCCCCCCCOC(=O)CCCCCN(CCO)CCCCCCCC(=O)N(CCCCCCCC)CCCCCCCC ; CCCCCCCCC(C)OC(=O)CCCCCCCN(CCO)CCCCCCCC(=O)N(CCCCCCCC)CCCCCCCC ; CCCCCCCCCOC(=O)CCCCCCCN(CCO)CCCCCCCOP(=O)(OCCCCCCCC)OCCCCCCCC]

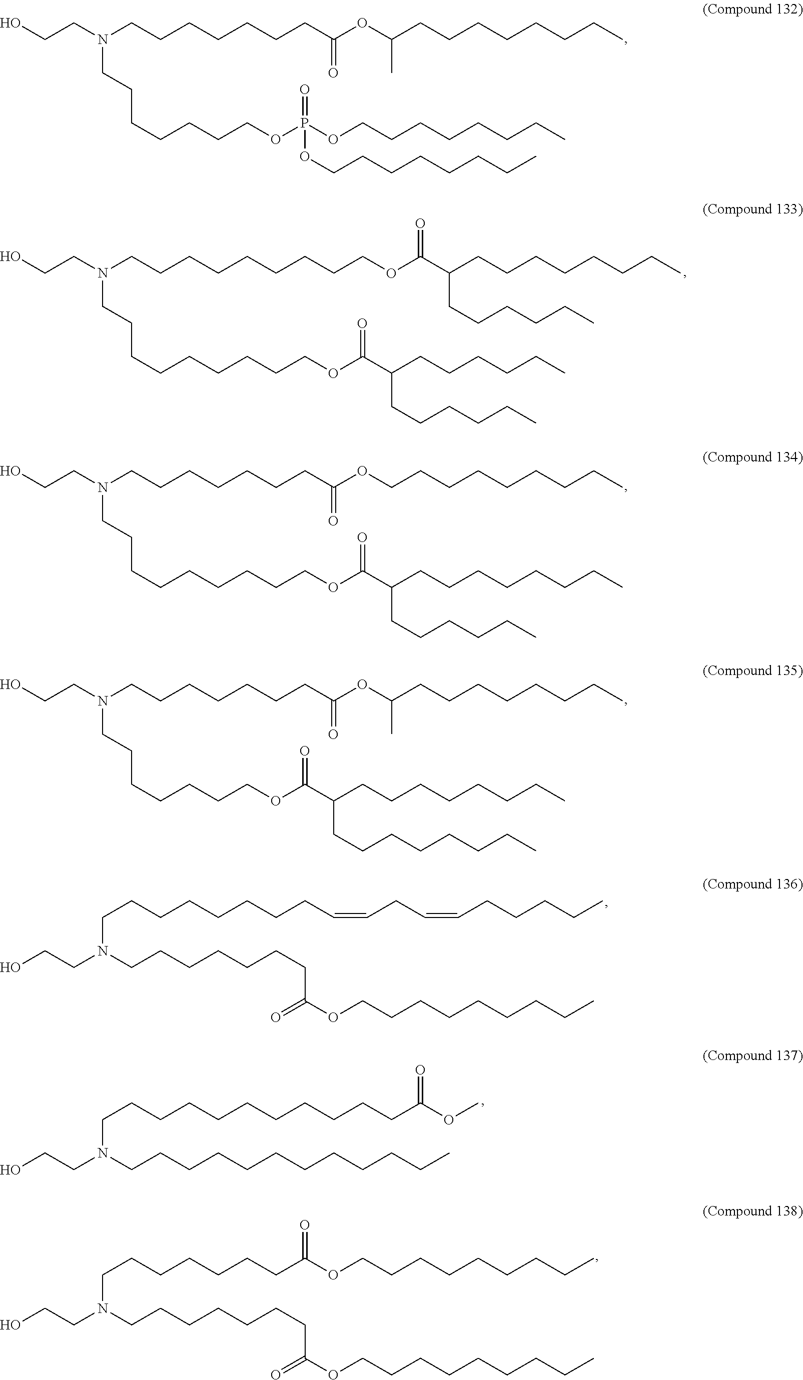

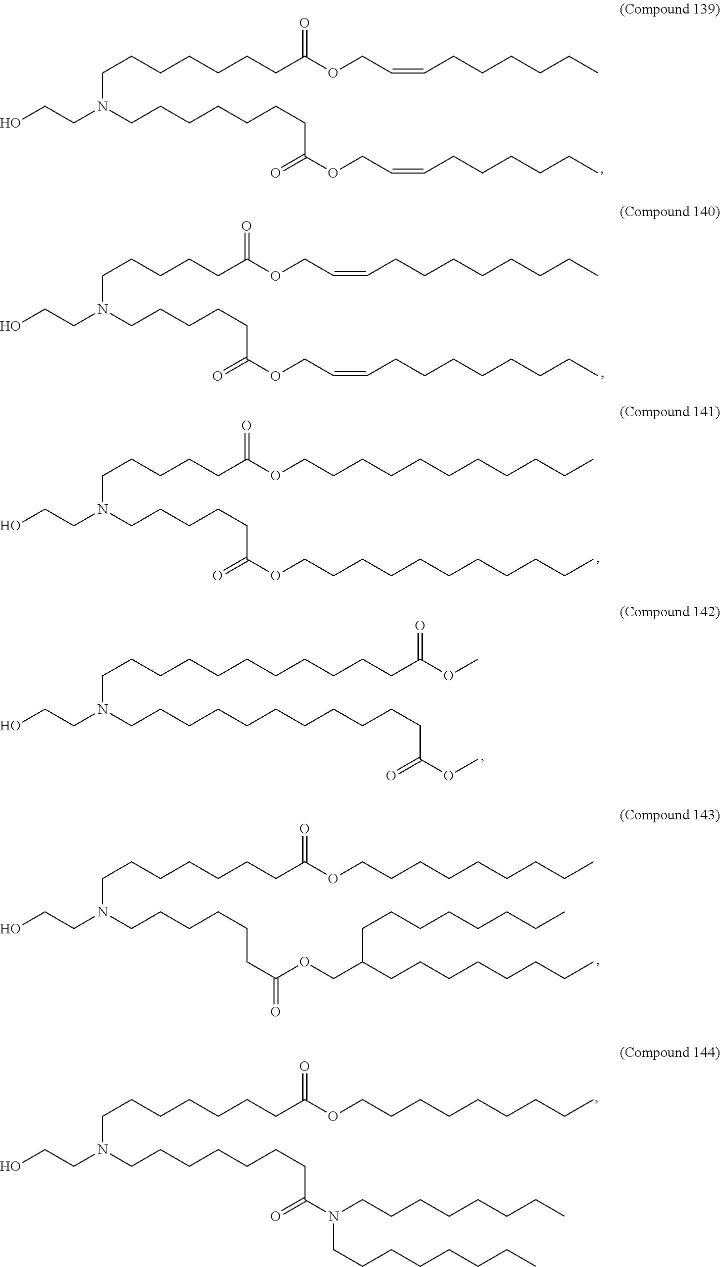 [SMILES: CCCCCC/C=C\COC(=O)CCCCCCCN(CCO)CCCCCCCC(=O)OC/C=C\CCCCCC ; CCCCCCCC/C=C\COC(=O)CCCCCN(CCO)CCCCCC(=O)OC/C=C\CCCCCCCC ; CCCCCCCCCCCOC(=O)CCCCCN(CCO)CCCCCC(=O)OCCCCCCCCCCC ; COC(=O)CCCCCCCCCCCN(CCO)CCCCCCCCCCCC(=O)OC ; CCCCCCCCCOC(=O)CCCCCCCN(CCO)CCCCCCC(=O)OCC(CCCCCCCC)CCCCCCCC ; CCCCCCCCCOC(=O)CCCCCCCN(CCO)CCCCCCCC(=O)N(CCCCCCCC)CCCCCCCC]

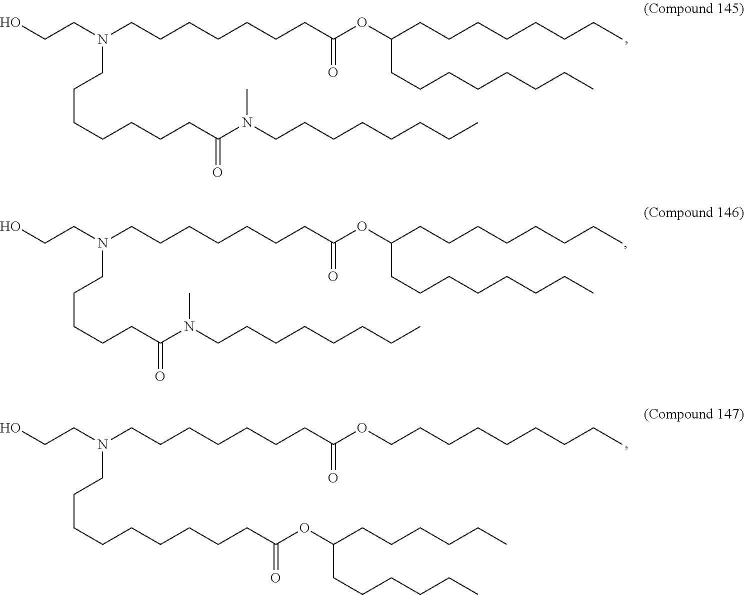 [SMILES: CCCCCCCCC(CCCCCCCC)OC(=O)CCCCCCCN(CCO)CCCCCCCC(=O)N(C)CCCCCCCC ; CCCCCCCCC(CCCCCCCC)OC(=O)CCCCCCCN(CCO)CCCCCC(=O)N(C)CCCCCCCC ; CCCCCCCCCOC(=O)CCCCCCCN(CCO)CCCCCCCCCC(=O)OC(CCCCCC)CCCCCC]

and salts or stereoisomers thereof.

The central amine moiety of a lipid according to formula (I) is typically protonated (i.e., positively charged) at a pH below the pKa of the amino moiety and is substantially not charged at a pH above the pKa. Such lipids can be referred to ionizable amino lipids.

In one specific embodiment, the compound of formula (I) is Compound 18.

In some embodiments, the amount the compound of formula (I) ranges from about 1 mol % to 99 mol % in the lipid composition.

In one embodiment, the amount of compound of formula (I) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the compound of formula (I) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the compound of formula (I) is about 50 mol % in the lipid composition.

In addition to the compound of formula (I), the lipid composition of the pharmaceutical compositions disclosed herein can comprise additional components such as phospholipids, structural lipids, quaternary amine compounds, PEG-lipids, and any combination thereof.

b. Additional Components in the Lipid Composition

(i) Phospholipids

The lipid composition of the pharmaceutical composition disclosed herein can comprise one or more phospholipids, for example, one or more saturated or (poly)unsaturated phospholipids or a combination thereof. In general, phospholipids comprise a phospholipid moiety and one or more fatty acid moieties. For example, a phospholipid can be a lipid according to formula (III):

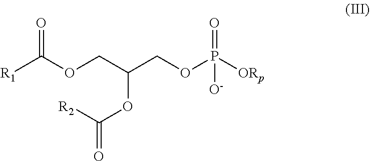

in which Rₚ represents a phospholipid moiety and R₁ and R₂ represent fatty acid moieties with or without unsaturation that can be the same or different.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue (e.g., tumoral tissue).

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, a pharmaceutical composition for intratumoral delivery disclosed herein can comprise more than one phospholipid. When more than one phospholipid is used, such phospholipids can belong to the same phospholipid class (e.g., MSPC and DSPC) or different classes (e.g., MSPC and MSPE).

Phospholipids can be of a symmetric or an asymmetric type. As used herein, the term “symmetric phospholipid” includes glycerophospholipids having matching fatty acid moieties and sphingolipids in which the variable fatty acid moiety and the hydrocarbon chain of the sphingosine backbone include a comparable number of carbon atoms. As used herein, the term “asymmetric phospholipid” includes lysolipids, glycerophospholipids having different fatty acid moieties (e.g., fatty acid moieties with different numbers of carbon atoms and/or unsaturations (e.g., double bonds)), and sphingolipids in which the variable fatty acid moiety and the hydrocarbon chain of the sphingosine backbone include a dissimilar number of carbon atoms (e.g., the variable fatty acid moiety include at least two more carbon atoms than the hydrocarbon chain or at least two fewer carbon atoms than the hydrocarbon chain).

In some embodiments, the lipid composition of a pharmaceutical composition disclosed herein comprises at least one symmetric phospholipid. Symmetric phospholipids can be selected from the non-limiting group consisting of
1,2-dipropionyl-sn-glycero-3-phosphocholine (03:0 PC),
1,2-dibutyryl-sn-glycero-3-phosphocholine (04:0 PC),
1,2-dipentanoyl-sn-glycero-3-phosphocholine (05:0 PC),
1,2-dihexanoyl-sn-glycero-3-phosphocholine (06:0 PC),
1,2-diheptanoyl-sn-glycero-3-phosphocholine (07:0 PC),
1,2-dioctanoyl-sn-glycero-3-phosphocholine (08:0 PC),
1,2-dinonanoyl-sn-glycero-3-phosphocholine (09:0 PC),
1,2-didecanoyl-sn-glycero-3-phosphocholine (10:0 PC),
1,2-diundecanoyl-sn-glycero-3-phosphocholine (11:0 PC, DUPC),
1,2-dilauroyl-sn-glycero-3-phosphocholine (12:0 PC),
1,2-ditridecanoyl-sn-glycero-3-phosphocholine (13:0 PC),
1,2-dimyristoyl-sn-glycero-3-phosphocholine (14:0 PC, DMPC),
1,2-dipentadecanoyl-sn-glycero-3-phosphocholine (15:0 PC),
1,2-dipalmitoyl-sn-glycero-3-phosphocholine (16:0 PC, DPPC),
1,2-diphytanoyl-sn-glycero-3-phosphocholine (4ME 16:0 PC),
1,2-diheptadecanoyl-sn-glycero-3-phosphocholine (17:0 PC),
1,2-distearoyl-sn-glycero-3-phosphocholine (18:0 PC, DSPC),
1,2-dinonadecanoyl-sn-glycero-3-phosphocholine (19:0 PC),
1,2-diarachidoyl-sn-glycero-3-phosphocholine (20:0 PC),
1,2-dihenarachidoyl-sn-glycero-3-phosphocholine (21:0 PC),
1,2-dibehenoyl-sn-glycero-3-phosphocholine (22:0 PC),
1,2-ditricosanoyl-sn-glycero-3-phosphocholine (23:0 PC),
1,2-dilignoceroyl-sn-glycero-3-phosphocholine (24:0 PC),
1,2-dimyristoleoyl-sn-glycero-3-phosphocholine (14:1 (Δ9-Cis) PC),
1,2-dimyristelaidoyl-sn-glycero-3-phosphocholine (14:1 (Δ9-Trans) PC),
1,2-dipalmitoleoyl-sn-glycero-3-phosphocholine (16:1 (Δ9-Cis) PC),
1,2-dipalmitelaidoyl-sn-glycero-3-phosphocholine (16:1 (Δ9-Trans) PC),
1,2-dipetroselenoyl-sn-glycero-3-phosphocholine (18:1 (A6-Cis) PC),
1,2-dioleoyl-sn-glycero-3-phosphocholine (18:1 (Δ9-Cis) PC, DOPC),
1,2-dielaidoyl-sn-glycero-3-phosphocholine (18:1 (Δ9-Trans) PC),
1,2-dilinoleoyl-sn-glycero-3-phosphocholine (18:2 (Cis) PC, DLPC),
1,2-dilinolenoyl-sn-glycero-3-phosphocholine (18:3 (Cis) PC, DLnPC),
1,2-dieicosenoyl-sn-glycero-3-phosphocholine (20:1 (Cis) PC),
1,2-diarachidonoyl-sn-glycero-3-phosphocholine (20:4 (Cis) PC, DAPC),
1,2-dierucoyl-sn-glycero-3-phosphocholine (22:1 (Cis) PC),
1,2-didocosahexaenoyl-sn-glycero-3-phosphocholine (22:6 (Cis) PC, DHAPC),
1,2-dinervonoyl-sn-glycero-3-phosphocholine (24:1 (Cis) PC),
1,2-dihexanoyl-sn-glycero-3-phosphoethanolamine (06:0 PE),
1,2-dioctanoyl-sn-glycero-3-phosphoethanolamine (08:0 PE),
1,2-didecanoyl-sn-glycero-3-phosphoethanolamine (10:0 PE),
1,2-dilauroyl-sn-glycero-3-phosphoethanolamine (12:0 PE),
1,2-dimyristoyl-sn-glycero-3-phosphoethanolamine (14:0 PE),
1,2-dipentadecanoyl-sn-glycero-3-phosphoethanolamine (15:0 PE),
1,2-dipalmitoyl-sn-glycero-3-phosphoethanolamine (16:0 PE),
1,2-diphytanoyl-sn-glycero-3-phosphoethanolamine (4ME 16:0 PE),
1,2-diheptadecanoyl-sn-glycero-3-phosphoethanolamine (17:0 PE),
1,2-distearoyl-sn-glycero-3-phosphoethanolamine (18:0 PE, DSPE),
1,2-dipalmitoleoyl-sn-glycero-3-phosphoethanolamine (16:1 PE),
1,2-dioleoyl-sn-glycero-3-phosphoethanolamine (18:1 (Δ9-Cis) PE, DOPE),
1,2-dielaidoyl-sn-glycero-3-phosphoethanolamine (18:1 (Δ9-Trans) PE),
1,2-dilinoleoyl-sn-glycero-3-phosphoethanolamine (18:2 PE, DLPE),
1,2-dilinolenoyl-sn-glycero-3-phosphoethanolamine (18:3 PE, DLnPE),
1,2-diarachidonoyl-sn-glycero-3-phosphoethanolamine (20:4 PE, DAPE),
1,2-didocosahexaenoyl-sn-glycero-3-phosphoethanolamine (22:6 PE, DHAPE),
1,2-di-O-octadecenyl-sn-glycero-3-phosphocholine (18:0 Diether PC),
1,2-dioleoyl-sn-glycero-3-phospho-rac-(1-glycerol) sodium salt (DOPG), and any combination thereof.

In some embodiments, the lipid composition of a pharmaceutical composition disclosed herein comprises at least one symmetric phospholipid selected from the non-limiting group consisting of DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof.

In some embodiments, the lipid composition of a pharmaceutical composition disclosed herein comprises at least one asymmetric phospholipid. Asymmetric phospholipids can be selected from the non-limiting group consisting of
1-myristoyl-2-palmitoyl-sn-glycero-3-phosphocholine (14:0-16:0 PC, MPPC),
1-myristoyl-2-stearoyl-sn-glycero-3-phosphocholine (14:0-18:0 PC, MSPC),
1-palmitoyl-2-acetyl-sn-glycero-3-phosphocholine (16:0-02:0 PC),
1-palmitoyl-2-myristoyl-sn-glycero-3-phosphocholine (16:0-14:0 PC, PMPC),
1-palmitoyl-2-stearoyl-sn-glycero-3-phosphocholine (16:0-18:0 PC, PSPC),
1-palmitoyl-2-oleoyl-sn-glycero-3-phosphocholine (16:0-18:1 PC, POPC),
1-palmitoyl-2-linoleoyl-sn-glycero-3-phosphocholine (16:0-18:2 PC, PLPC),
1-palmitoyl-2-arachidonoyl-sn-glycero-3-phosphocholine (16:0-20:4 PC),
1-palmitoyl-2-docosahexaenoyl-sn-glycero-3-phosphocholine (14:0-22:6 PC),
1-stearoyl-2-myristoyl-sn-glycero-3-phosphocholine (18:0-14:0 PC, SMPC),
1-stearoyl-2-palmitoyl-sn-glycero-3-phosphocholine (18:0-16:0 PC, SPPC),
1-stearoyl-2-oleoyl-sn-glycero-3-phosphocholine (18:0-18:1 PC, SOPC),
1-stearoyl-2-linoleoyl-sn-glycero-3-phosphocholine (18:0-18:2 PC),
1-stearoyl-2-arachidonoyl-sn-glycero-3-phosphocholine (18:0-20:4 PC),
1-stearoyl-2-docosahexaenoyl-sn-glycero-3-phosphocholine (18:0-22:6 PC),
1-oleoyl-2-myristoyl-sn-glycero-3-phosphocholine (18:1-14:0 PC, OMPC),
1-oleoyl-2-palmitoyl-sn-glycero-3-phosphocholine (18:1-16:0 PC, OPPC),
1-oleoyl-2-stearoyl-sn-glycero-3-phosphocholine (18:1-18:0 PC, OSPC),
1-palmitoyl-2-oleoyl-sn-glycero-3-phosphoethanolamine (16:0-18:1 PE, POPE),
1-palmitoyl-2-linoleoyl-sn-glycero-3-phosphoethanolamine (16:0-18:2 PE),
1-palmitoyl-2-arachidonoyl-sn-glycero-3-phosphoethanolamine (16:0-20:4 PE),
1-palmitoyl-2-docosahexaenoyl-sn-glycero-3-phosphoethanolamine (16:0-22:6 PE),
1-stearoyl-2-oleoyl-sn-glycero-3-phosphoethanolamine (18:0-18:1 PE),
1-stearoyl-2-linoleoyl-sn-glycero-3-phosphoethanolamine (18:0-18:2 PE),
1-stearoyl-2-arachidonoyl-sn-glycero-3-phosphoethanolamine (18:0-20:4 PE),
1-stearoyl-2-docosahexaenoyl-sn-glycero-3-phosphoethanolamine (18:0-22:6 PE),
1-oleoyl-2-cholesterylhemisuccinoyl-sn-glycero-3-phosphocholine (OChemsPC), and
any combination thereof.

Asymmetric lipids useful in the lipid composition can also be lysolipids. Lysolipids can be selected from the non-limiting group consisting of
1-hexanoyl-2-hydroxy-sn-glycero-3-phosphocholine (06:0 Lyso PC),
1-heptanoyl-2-hydroxy-sn-glycero-3-phosphocholine (07:0 Lyso PC),
1-octanoyl-2-hydroxy-sn-glycero-3-phosphocholine (08:0 Lyso PC),
1-nonanoyl-2-hydroxy-sn-glycero-3-phosphocholine (09:0 Lyso PC),
1-decanoyl-2-hydroxy-sn-glycero-3-phosphocholine (10:0 Lyso PC),
1-undecanoyl-2-hydroxy-sn-glycero-3-phosphocholine (11:0 Lyso PC),
1-lauroyl-2-hydroxy-sn-glycero-3-phosphocholine (12:0 Lyso PC),
1-tridecanoyl-2-hydroxy-sn-glycero-3-phosphocholine (13:0 Lyso PC),
1-myristoyl-2-hydroxy-sn-glycero-3-phosphocholine (14:0 Lyso PC),
1-pentadecanoyl-2-hydroxy-sn-glycero-3-phosphocholine (15:0 Lyso PC),
1-palmitoyl-2-hydroxy-sn-glycero-3-phosphocholine (16:0 Lyso PC),
1-heptadecanoyl-2-hydroxy-sn-glycero-3-phosphocholine (17:0 Lyso PC),
1-stearoyl-2-hydroxy-sn-glycero-3-phosphocholine (18:0 Lyso PC),
1-oleoyl-2-hydroxy-sn-glycero-3-phosphocholine (18:1 Lyso PC),
1-nonadecanoyl-2-hydroxy-sn-glycero-3-phosphocholine (19:0 Lyso PC),
1-arachidoyl-2-hydroxy-sn-glycero-3-phosphocholine (20:0 Lyso PC),
1-behenoyl-2-hydroxy-sn-glycero-3-phosphocholine (22:0 Lyso PC),
1-lignoceroyl-2-hydroxy-sn-glycero-3-phosphocholine (24:0 Lyso PC),
1-hexacosanoyl-2-hydroxy-sn-glycero-3-phosphocholine (26:0 Lyso PC),
1-myristoyl-2-hydroxy-sn-glycero-3-phosphoethanolamine (14:0 Lyso PE),
1-palmitoyl-2-hydroxy-sn-glycero-3-phosphoethanolamine (16:0 Lyso PE),
1-stearoyl-2-hydroxy-sn-glycero-3-phosphoethanolamine (18:0 Lyso PE),
1-oleoyl-2-hydroxy-sn-glycero-3-phosphoethanolamine (18:1 Lyso PE),
1-hexadecyl-sn-glycero-3-phosphocholine (C16 Lyso PC), and
any combination thereof.

In some embodiment, the lipid composition of a pharmaceutical composition disclosed herein comprises at least one asymmetric phospholipid selected from the group consisting of MPPC, MSPC, PMPC, PSPC, SMPC, SPPC, and any combination thereof. In some embodiments, the asymmetric phospholipid is 1-myristoyl-2-stearoyl-sn-glycero-3-phosphocholine (MSPC).

In some embodiments, the lipid compositions disclosed herein can contain one or more symmetric phospholipids, one or more asymmetric phospholipids, or a combination thereof. When multiple phospholipids are present, they can be present in equimolar ratios, or non-equimolar ratios.

In one embodiment, the lipid composition of a pharmaceutical composition disclosed herein comprises a total amount of phospholipid (e.g., MSPC) which ranges from about 1 mol % to about 20 mol %, from about 5 mol % to about 20 mol %, from about 10 mol % to about 20 mol %, from about 15 mol % to about 20 mol %, from about 1 mol % to about 15 mol %, from about 5 mol % to about 15 mol %, from about 10 mol % to about 15 mol %, from about 5 mol % to about 10 mol % in the lipid composition. In one embodiment, the amount of the phospholipid is from about 8 mol % to about 15 mol % in the lipid composition. In one embodiment, the amount of the phospholipid (e.g., MSPC) is about 10 mol % in the lipid composition.

In some aspects, the amount of a specific phospholipid (e.g., MSPC) is at least about 1, 2, 3, 4, 5, 6, 7, 8, 9, 10, 11, 12, 13, 14, 15, 16, 17, 18, 19 or 20 mol % in the lipid composition.

(ii) Quaternary Amine Compounds

The lipid composition of a pharmaceutical composition disclosed herein can comprise one or more quaternary amine compounds (e.g., DOTAP). The term “quaternary amine compound” is used to include those compounds having one or more quaternary amine groups (e.g., trialkylamino groups) and permanently carrying a positive charge and existing in a form of a salt. For example, the one or more quaternary amine groups can be present in a lipid or a polymer (e.g., PEG). In some embodiments, the quaternary amine compound comprises (1) a quaternary amine group and (2) at least one hydrophobic tail group comprising (i) a hydrocarbon chain, linear or branched, and saturated or unsaturated, and (ii) optionally an ether, ester, carbonyl, or ketal linkage between the quaternary amine group and the hydrocarbon chain. In some embodiments, the quaternary amine group can be a trimethylammonium group. In some embodiments, the quaternary amine compound comprises two identical hydrocarbon chains. In some embodiments, the quaternary amine compound comprises two different hydrocarbon chains.

In some embodiments, the lipid composition of a pharmaceutical composition disclosed herein comprises at least one quaternary amine compound. Quaternary amine compound can be selected from the non-limiting group consisting of
1,2-dioleoyl-3-trimethylammonium-propane (DOTAP),
N-[1-(2,3-dioleoyloxy)propyl]-N,N,N-trimethylammonium chloride (DOTMA),
1-[2-(oleoyloxy)ethyl]-2-oleyl-3-(2-hydroxyethyl)imidazolinium chloride (DOTIM),
2,3-dioleyloxy-N-[2(sperminecarboxamido)ethyl]-N,N-dimethyl-1-propanaminium trifluoroacetate (DOSPA),
N,N-distearyl-N,N-dimethylammonium bromide (DDAB),
N-(1,2-dimyristyloxyprop-3-yl)-N,N-dimethyl-N-hydroxyethyl ammonium bromide (DMRIE),
N-(1,2-dioleoyloxyprop-3-yl)-N,N-dimethyl-N-hydroxyethyl ammonium bromide (DORIE),
N,N-dioleyl-N,N-dimethylammonium chloride (DODAC),
1,2-dilauroyl-sn-glycero-3-ethylphosphocholine (DLePC),
1,2-distearoyl-3-trimethylammonium-propane (DSTAP),
1,2-dipalmitoyl-3-trimethylammonium-propane (DPTAP),
1,2-dilinoleoyl-3-trimethylammonium-propane (DLTAP),
1,2-dimyristoyl-3-trimethylammonium-propane (DMTAP)
1,2-distearoyl-sn-glycero-3-ethylphosphocholine (DSePC)
1,2-dipalmitoyl-sn-glycero-3-ethylphosphocholine (DPePC),
1,2-dimyristoyl-sn-glycero-3-ethylphosphocholine (DMePC),
1,2-dioleoyl-sn-glycero-3-ethylphosphocholine (DOePC),
1,2-di-(9Z-tetradecenoyl)-sn-glycero-3-ethylphosphocholine (14:1 EPC),
1-palmitoyl-2-oleoyl-sn-glycero-3-ethylphosphocholine (16:0-18:1 EPC),
and any combination thereof.

In one embodiment, the quaternary amine compound is 1,2-dioleoyl-3-trimethylammonium-propane (DOTAP).

Quaternary amine compounds are known in the art, such as those described in US 2013/0245107 A1, US 2014/0363493 A1, U.S. Pat. No. 8,158,601, WO 2015/123264 A1, and WO 2015/148247 A1, which are incorporated herein by reference in their entirety.

In one embodiment, the amount of the quaternary amine compound (e.g., DOTAP) in the lipid composition disclosed herein ranges from about 0.01 mol % to about 20 mol %.

In one embodiment, the amount of the quaternary amine compound (e.g., DOTAP) in the lipid composition disclosed herein ranges from about 0.5 mol % to about 20 mol %, from about 0.5 mol % to about 15 mol %, from about 0.5 mol % to about 10 mol %, from about 1 mol % to about 20 mol %, from about 1 mol % to about 15 mol %, from about 1 mol % to about 10 mol %, from about 2 mol % to about 20 mol %, from about 2 mol % to about 15 mol %, from about 2 mol % to about 10 mol %, from about 3 mol % to about 20 mol %, from about 3 mol % to about 15 mol %, from about 3 mol % to about 10 mol %, from about 4 mol % to about 20 mol %, from about 4 mol % to about 15 mol %, from about 4 mol % to about 10 mol %, from about 5 mol % to about 20 mol %, from about 5 mol % to about 15 mol %, from about 5 mol % to about 10 mol %, from about 6 mol % to about 20 mol %, from about 6 mol % to about 15 mol %, from about 6 mol % to about 10 mol %, from about 7 mol % to about 20 mol %, from about 7 mol % to about 15 mol %, from about 7 mol % to about 10 mol %, from about 8 mol % to about 20 mol %, from about 8 mol % to about 15 mol %, from about 8 mol % to about 10 mol %, from about 9 mol % to about 20 mol %, from about 9 mol % to about 15 mol %, from about 9 mol % to about 10 mol %.

In one embodiment, the amount of the quaternary amine compound (e.g., DOTAP) in the lipid composition disclosed herein ranges from about 5 mol % to about 10 mol %.

In one embodiment, the amount of the quaternary amine compound (e.g., DOTAP) in the lipid composition disclosed herein is about 5 mol %. In one embodiment, the amount of the quaternary amine compound (e.g., DOTAP) in the lipid composition disclosed herein is about 10 mol %.

In some embodiments, the amount of the quaternary amine compound (e.g., DOTAP) is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mol % in the lipid composition disclosed herein.

In one embodiment, the mole ratio of the compound of formula (I) (e.g., Compounds 18, 25, 26 or 48) to the quaternary amine compound (e.g., DOTA) is about 100:1 to about 2.5:1. In one embodiment, the mole ratio of the compound of formula (I) (e.g., Compounds 18, 25, 26 or 48) to the quaternary amine compound (e.g., DOTAP) is about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, or about 2.5:1. In one embodiment, the mole ratio of the compound of formula (I) (e.g., Compounds 18, 25, 26 or 48) to the quaternary amine compound (e.g., DOTAP) in the lipid composition disclosed herein is about 10:1.

In some aspects, the lipid composition the pharmaceutical compositions disclosed herein does not comprise a quaternary amine compound. In some aspects, the lipid composition of the pharmaceutical compositions disclosed does not comprise DOTAP.

(iii) Structural Lipids

The lipid composition of a pharmaceutical composition disclosed herein can comprise one or more structural lipids. As used herein, the term “structural lipid” refers to sterols and also to lipids containing sterol moieties. In some embodiments, the structural lipid is selected from the group consisting of cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol.

In one embodiment, the amount of the structural lipid (e.g., an sterol such as cholesterol) in the lipid composition of a pharmaceutical composition disclosed herein ranges from about 20 mol % to about 60 mol %, from about 25 mol % to about 55 mol %, from about 30 mol % to about 50 mol %, or from about 35 mol % to about 45 mol %.

In one embodiment, the amount of the structural lipid (e.g., an sterol such as cholesterol) in the lipid composition disclosed herein ranges from about 25 mol % to about 30 mol %, from about 30 mol % to about 35 mol %, or from about 35 mol % to about 40 mol %.

In one embodiment, the amount of the structural lipid (e.g., a sterol such as cholesterol) in the lipid composition disclosed herein is about 23.5 mol %, about 28.5 mol %, about 33.5 mol %, or about 38.5 mol %.

In some embodiments, the amount of the structural lipid (e.g., an sterol such as cholesterol) in the lipid composition disclosed herein is at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

In some aspects, the lipid composition component of the pharmaceutical compositions for intratumoral delivery disclosed does not comprise cholesterol.

(iv) Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol % mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

In some aspects, the lipid composition of the pharmaceutical compositions disclosed herein does not comprise a PEG-lipid.

In some embodiments, the lipid composition disclosed herein comprises a compound of formula (I) and an asymmetric phospholipid. In some embodiments, the lipid composition comprises compound 18 and MSPC.

In some embodiments, the lipid composition disclosed herein comprises a compound of formula (I) and a quaternary amine compound. In some embodiments, the lipid composition comprises compound 18 and DOTAP.

In some embodiments, the lipid composition disclosed herein comprises a compound of formula (I), an asymmetric phospholipid, and a quaternary amine compound. In some embodiments, the lipid composition comprises compound 18, MSPC and DOTAP.

In one embodiment, the lipid composition comprises about 50 mol % of a compound of formula (I) (e.g. Compounds 18, 25, 26 or 48), about 10 mol % of DSPC or MSPC, about 33.5 mol % of cholesterol, about 1.5 mol % of PEG-DMG, and about 5 mol % of DOTAP.

In one embodiment, the lipid composition comprises about 50 mol % of a compound of formula (I) (e.g. Compounds 18, 25, 26 or 48), about 10 mol % of DSPC or MSPC, about 28.5 mol % of cholesterol, about 1.5 mol % of PEG-DMG, and about 10 mol % of DOTAP.

The components of the lipid nanoparticle can be tailored for optimal delivery of the polynucleotides based on the desired outcome. As a non-limiting example, the lipid nanoparticle can comprise 40-60 mol % a compound of formula (I), 8-16 mol % phospholipid, 30-45 mol % cholesterol, 1-5 mol % PEG lipid, and optionally 1-15 mol % quaternary amine compound.

In some embodiments, the lipid nanoparticle can comprise 45-65 mol % of a compound of formula (I), 5-10 mol % phospholipid, 25-40 mol % cholesterol, 0.5-5 mol % PEG lipid, and optionally 1-15 mol % quaternary amine compound.

Non-limiting examples of nucleic acid lipid particles are disclosed in U.S. Patent Publication No. 20140121263, herein incorporated by reference in its entirety.

(v) Other Ionizable Amino Lipids

The lipid composition of the pharmaceutical composition disclosed herein can comprise one or more ionizable amino lipids in addition to a lipid according to formula (I).

Ionizable lipids can be selected from the non-limiting group consisting of
3-(didodecylamino)-N1,N1,4-tridodecyl-1-piperazineethanamine (KL10),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA),
heptatriaconta-6,9,28,31-tetraen-19-yl 4-(dimethylamino)butanoate (DLin-MC3-DMA),
2,2-dilinoleyl-4-(2-dimethylaminoethyl)-[1,3]-dioxolane (DLin-KC2-DMA),
1,2-dioleyloxy-N,N-dimethylaminopropane (DODMA), (13Z,165Z)—N,N-dimethyl-3-nonydocosa-13-16-dien-1-amine (L608),
2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA),
(2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and
(2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids can also be the compounds disclosed in International Publication No. WO 2015/199952 A1, hereby incorporated by reference in its entirety. For example, the ionizable amino lipids include, but not limited to:

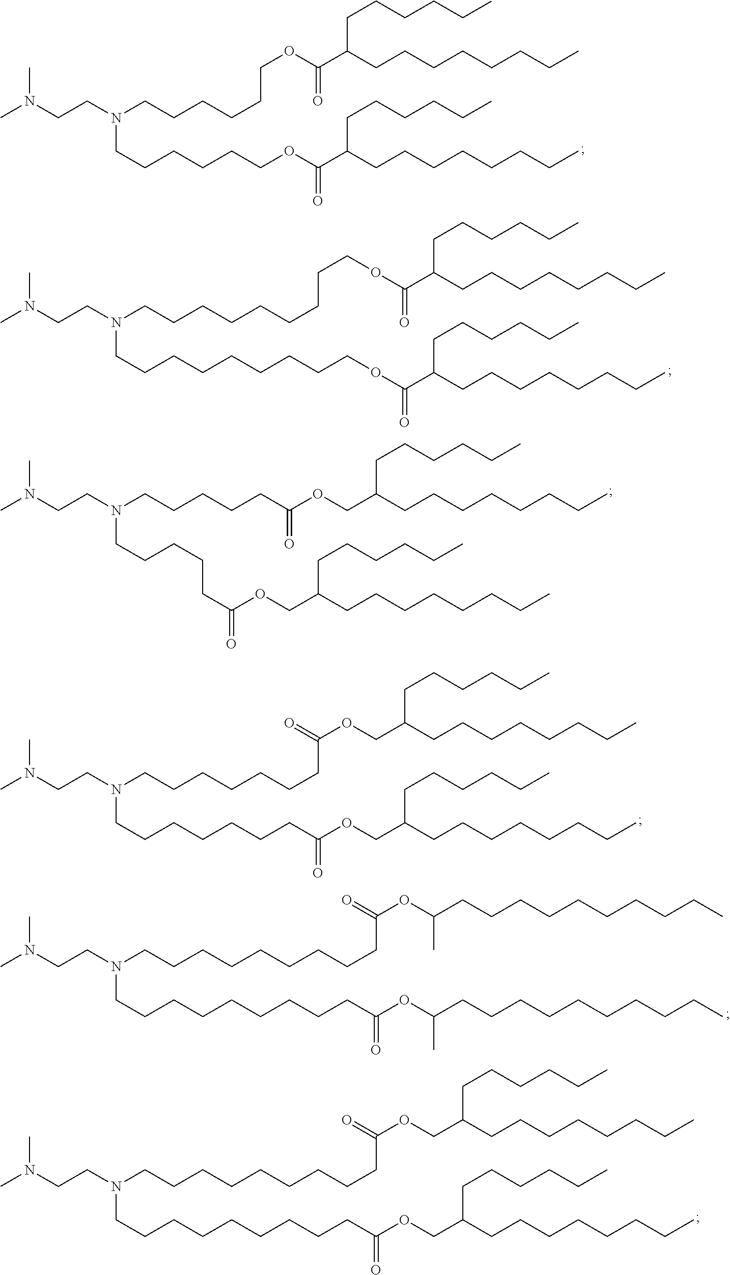

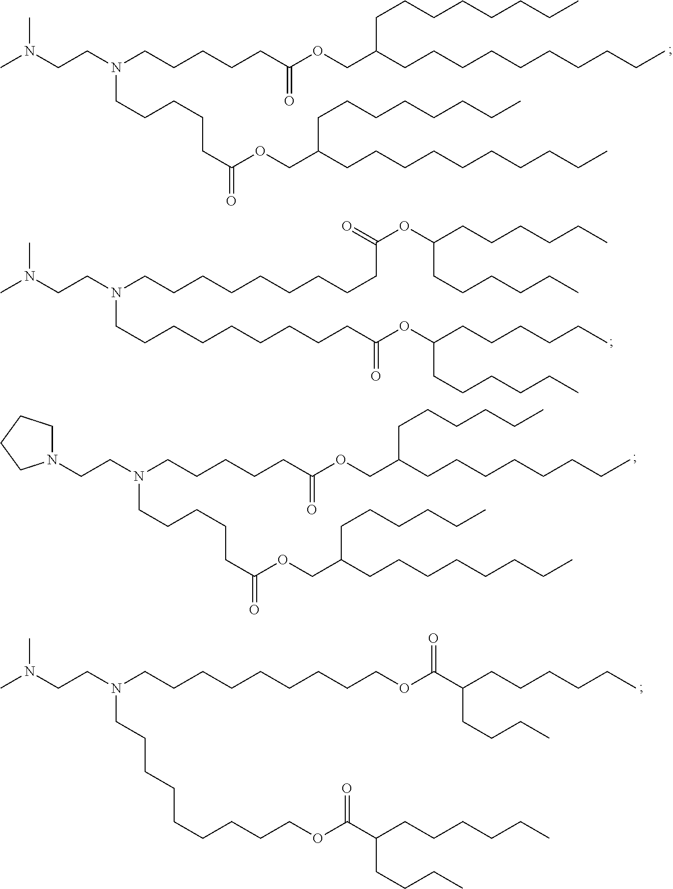

and any combination thereof.

(vi) Other Lipid Composition Components

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof). The lipid composition can include a buffer such as, but not limited to, citrate or phosphate at a pH of 7, salt and/or sugar. Salt and/or sugar can be included in the formulations described herein for isotonicity.

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

The ratio between the lipid composition and the polynucleotide range can be from about 10:1 to about 60:1 (wt/wt).

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In some embodiments, the pharmaceutical composition disclosed herein can contain more than one polypeptides. For example, a pharmaceutical composition disclosed herein can contain two or more polynucleotides (e.g., RNA, e.g., mRNA).

In one embodiment, the lipid nanoparticles described herein can comprise polynucleotides (e.g., mRNA) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

In one embodiment, formulations comprising the polynucleotides and lipid nanoparticles described herein can comprise 0.15 mg/ml to 2 mg/ml of the polynucleotide described herein (e.g., mRNA). In some embodiments, the formulation can further comprise 10 mM of citrate buffer and the formulation can additionally comprise up to 10% w/w of sucrose (e.g., at least 1% w/w, at least 2% w/w/, at least 3% w/w, at least 4% w/w, at least 5% w/w, at least 6% w/w, at least 7% w/w, at least 8% w/w, at least 9% w/w or 10% w/w).

(vii) Nanoparticle Compositions

In some embodiments, the pharmaceutical compositions disclosed herein are formulated as lipid nanoparticles (LNP). Accordingly, the present disclosure also provides nanoparticle compositions comprising (i) a lipid composition comprising a compound of formula (I) as described herein, and (ii) a polynucleotide encoding a polypeptide (e.g., SteA-BH3 polypeptide). In such nanoparticle composition, the lipid composition disclosed herein can encapsulate the polynucleotide encoding a polypeptide (e.g., SteA-BH3 polypeptide).

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

Nanoparticle compositions of the present disclosure comprise at least one compound according to formula (I). For example, the nanoparticle composition can include one or more of Compounds 1-147. Nanoparticle compositions can also include a variety of other components. For example, the nanoparticle composition can include one or more other lipids in addition to a lipid according to formula (I) or (II), for example (i) at least one phospholipid, (ii) at least one quaternary amine compound, (iii) at least one structural lipid, (iv) at least one PEG-lipid, or (v) any combination thereof.

In some embodiments, the nanoparticle composition comprises a compound of formula (I), (e.g., Compounds 18, 25, 26 or 48). In some embodiments, the nanoparticle composition comprises a compound of formula (I) (e.g., Compounds 18, 25, 26 or 48) and a phospholipid (e.g., DSPC or MSPC). In some embodiments, the nanoparticle composition comprises a compound of formula (I) (e.g., Compounds 18, 25, 26 or 48), a phospholipid (e.g., DSPC or MSPC), and a quaternary amine compound (e.g., DOTAP). In some embodiments, the nanoparticle composition comprises a compound of formula (I) (e.g., Compounds 18, 25, 26 or 48), and a quaternary amine compound (e.g., DOTAP).

In some embodiments, the nanoparticle composition comprises a lipid composition consisting or consisting essentially of compound of formula (I) (e.g., Compounds 18, 25, 26 or 48). In some embodiments, the nanoparticle composition comprises a lipid composition consisting or consisting essentially of a compound of formula (I) (e.g., Compounds 18, 25, 26 or 48) and a phospholipid (e.g., DSPC or MSPC). In some embodiments, the nanoparticle composition comprises a lipid composition consisting or consisting essentially of a compound of formula (I) (e.g., Compounds 18, 25, 26 or 48), a phospholipid (e.g., DSPC or MSPC), and a quaternary amine compound (e.g., DOTAP). In some embodiments, the nanoparticle composition comprises a lipid composition consisting or consisting essentially of a compound of formula (I) (e.g., Compounds 18, 25, 26 or 48), and a quaternary amine compound (e.g., DOTAP).

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding a polypeptide (e.g., SteA-BH3 polypeptide) is formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1,3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16: 940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

### Pharmaceutical Compositions

The present invention includes pharmaceutical compositions comprising an mRNA or a nanoparticle (e.g., a lipid nanoparticle) described herein, in combination with one or more pharmaceutically acceptable excipient, carrier or diluent. In some embodiments, a pharmaceutical composition of the invention includes an mRNA encoding one or more BH3 domains. In certain embodiments, a pharmaceutical composition of the invention includes an mRNA encoding a Bcl-2-like polypeptide or fragment or variant thereof. In certain embodiments, a pharmaceutical composition includes both a first mRNA encoding one or more BH3 domains, and a second mRNA encoding a Bcl-2-like polypeptide or fragment or variant thereof. In particular embodiments, the mRNA is present in a nanoparticle, e.g., a lipid nanoparticle. In particular embodiments, the mRNA or nanoparticle is present in a pharmaceutical composition. In various embodiments, the one or more mRNA present in the pharmaceutical composition is encapsulated in a nanoparticle, e.g., a lipid nanoparticle. In particular embodiments, the molar ratio of the first mRNA to the second mRNA is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In particular embodiments, the molar ratio of the first mRNA to the second mRNA is greater than 1:1.

Pharmaceutical compositions may optionally include one or more additional active substances, for example, therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference in its entirety). In particular embodiments, a pharmaceutical composition comprises an mRNA and a lipid nanoparticle, or complexes thereof. In some embodiments, the pharmaceutical compositions or formulations described herein may contain at least one mRNA encoding one or more intracellular binding domains as described herein. In particular embodiments, the pharmaceutical compositions or formulations described herein may contain at least one mRNA encoding one or more BH3 domains and/or at least one mRNA encoding a BH3-trap polypeptide (e.g., a Bcl-2-like polypeptide).

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may include between 0.1% and 100%, e.g., between 0.5% and 70%, between 1% and 30%, between 5% and 80%, or at least 80% (w/w) active ingredient. In some embodiments, the active agent is an mRNA encoding one or more intracellular binding domains described herein. In some embodiments, the active agent is an mRNA encoding one or more BH3 domains.

The mRNAs of the invention (e.g., mRNA encoding one or more BH3 domains and/or a BH3-trap polypeptide) can be formulated using one or more excipients to: (1) increase stability; (2) increase cell transfection; (3) permit the sustained or delayed release (e.g., from a depot formulation of the mRNA); (4) alter the biodistribution (e.g., target the mRNA to specific tissues or cell types); (5) increase the translation of a polypeptide encoded by the mRNA in vivo; and/or (6) alter the release profile of a polypeptide encoded by the mRNA in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles (e.g., liposomes and micelles), polymers, lipoplexes, core-shell nanoparticles, peptides, proteins, carbohydrates, cells transfected with mRNAs (e.g., for transplantation into a subject), hyaluronidase, nanoparticle mimics and combinations thereof. Accordingly, the formulations of the invention can include one or more excipients, each in an amount that together increases the stability of the mRNA, increases cell transfection by the mRNA, increases the expression of a polypeptide encoded by the mRNA, and/or alters the release profile of a mRNA-encoded polypeptide. Further, the mRNAs of the present invention may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Excipients may include, for example: antiadherents, antioxidants, binders, coatings, compression aids, disintegrants, dyes (colors), emollients, emulsifiers, fillers (diluents), film formers or coatings, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

In some embodiments, the formulations described herein may include at least one pharmaceutically acceptable salt. Examples of pharmaceutically acceptable salts that may be included in a formulation of the invention include, but are not limited to, acid addition salts, alkali or alkaline earth metal salts, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

In some embodiments, the formulations described herein may contain at least one type of polynucleotide. As a non-limiting example, the formulations may contain 1, 2, 3, 4, 5 or more than 5 mRNAs described herein. In some embodiments, the formulations described herein may contain at least one mRNA encoding one or more BH3 domains and/or a BH3-trap polypeptide (e.g., a Bcl-2-like polypeptide or variant or fragment thereof), and at least one nucleic acid sequence such as, but not limited to, an siRNA, an shRNA, a snoRNA, and an miRNA.

Liquid dosage forms for e.g., parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and/or suspending agents. In certain embodiments for parenteral administration, compositions are mixed with solubilizing agents such as CREMAPHOR®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, pharmaceutical compositions including at least one mRNA described herein are administered to mammals (e.g., humans). Although the descriptions of pharmaceutical compositions provided herein are principally directed to pharmaceutical compositions which are suitable for administration to humans, it will be understood by the skilled artisan that such compositions are generally suitable for administration to any other animal, e.g., to a non-human mammal. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys. In particular embodiments, a subject is provided with two or more mRNAs described herein, e.g., a first mRNA encoding one or more BH3 domains and a second mRNA encoding a Bcl-2-like polypeptide. In particular embodiments, the first and second mRNAs are provided to the subject at the same time or at different times, e.g., sequentially. In particular embodiments, the first and second mRNAs are provided to the subject in the same pharmaceutical composition or formulation, e.g., to facilitate uptake of both mRNAs by the same cells.

The present invention also includes kits comprising a container comprising a first mRNA encoding one or more BH3 domains and a second mRNA encoding a Bcl-2-like polypeptide or variant or fragment thereof, e.g., a prosurvival Bcl-2-like polypeptide. In other embodiments, the kit comprises a first container comprising the mRNA encoding one or more BH3 domains and a second container comprising the mRNA encoding the Bcl-2-like polypeptide or variant or fragment thereof. In particular embodiments, the mRNAs are present in the same or different nanoparticles and/or pharmaceutical compositions. In particular embodiments, the mRNAs are lyophilized, dried, or freeze-dried.

### Therapeutic Methods

The invention also provides methods of treating or preventing a cancer in a subject in need thereof that involve providing or administering an mRNA encoding one or more intracellular binding domains as described herein to the subject. In a related embodiment, the subject is provided with or administered a nanoparticle (e.g., a lipid nanoparticle) comprising the mRNA. In further related embodiments, the subject is provided with or administered a pharmaceutical composition of the invention to the subject. In particular embodiments, the pharmaceutical composition comprises an mRNA encoding a BH3 domain(s) as described herein, or it comprises a nanoparticle comprising the mRNA. In particular embodiments, the mRNA is present in a nanoparticle, e.g., a lipid nanoparticle. In particular embodiments, the mRNA or nanoparticle is present in a pharmaceutical composition. In certain embodiments, the subject in need thereof has been diagnosed with a cancer, or is considered to be at risk of developing a cancer. In some embodiments, the cancer is liver cancer or colorectal cancer. In particular embodiments, the liver cancer is hepatocellular carcinoma. In some embodiments, the colorectal cancer is a primary tumor or a metastasis. In some embodiments, the cancer is a hematopoetic cancer. In some embodiments, the cancer is an acute myeloid leukemia, a chronic myeloid leukemia, a chronic myelomonocytic leukemia, a myelodystrophic syndrome (including refractory anemias and refractory cytopenias) or a myeloproliferative neoplasm or disease (including polycythemia vera, essential thrombocytosis and primary myelofibrosis).

In other embodiments, the cancer is a blood-based cancer or a hematopoetic cancer. Selectivity for a particular cancer type can be achieved through the combination of use of an appropriate LNP formulation (e.g., targeting specific cell types) in combination with appropriate regulatory site(s) (e.g., microRNAs) engineered into the mRNA constructs.

In some embodiments, the mRNA, nanoparticle, or pharmaceutical composition is administered to the patient parenterally. In particular embodiments, the subject is a mammal, e.g., a human. In particular embodiments, the pharmaceutical composition comprises an mRNA encoding one or more BH3 domains, as described herein. In various embodiments, the subject is provided with an effective amount of the mRNA.

The invention further provides methods of treating or preventing cancer in a subject in need thereof, comprising providing the subject with an effective amount of an mRNA described herein, e.g., an mRNA encoding one or more BH3 domains, wherein the mRNA further comprises a regulatory element that enhances expression of the BH3 domain(s) in cancer cells as compared to normal cells. In particular embodiments, the regulatory element is a binding site for a microRNA that has greater expression in normal cells than cancer cells (e.g., a miR-122 binding site), wherein binding of the microRNA to the binding site inhibits expression of the intracellular binding domain(s) (e.g., BH3). In particular embodiments, the mRNA is present in a nanoparticle, e.g., a lipid nanoparticle. In particular embodiments, the mRNA or nanoparticle is present in a pharmaceutical composition. The nanoparticle or the isolated mRNA may be taken up and translated in the subject's cells to produce the one or more intracellular binding domains. In particular embodiments, expression of the intracellular binding domain(s) is greater in cancer cells than normal cells, resulting in greater apoptosis of cancer cells than normal cells.

In particular embodiments, the subject is provided or administered both a first mRNA encoding one or more BH3 domains and a second mRNA encoding a Bcl-2-like polypeptide or variant or fragment thereof. In particular embodiments, the first mRNA comprises a regulatory element to enhance expression of the BH3 domain(s) in cancer cells as compared to normal cells and/or the second mRNA comprises a regulatory element to enhance expression in normal cells as compared to cancer cells. In particular embodiments, the first mRNA comprises a miR-122 binding sequence. In particular embodiments, the second mRNA comprises a miR-21 binding sequence. In certain embodiments, the first mRNA and second mRNA are both present in the same nanoparticle or pharmaceutical composition. In certain embodiments, the first mRNA and the second mRNA are present in different nanoparticles or pharmaceutical compositions. In particular embodiments, the molar ratio of the first mRNA to the second mRNA is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In particular embodiments, the molar ration of the first mRNA to the second mRNA is greater than 1:1.

Thus, in some embodiments, the present invention includes a method of treating or preventing cancer in a subject in need thereof, comprising providing to the subject a first mRNA described herein, e.g., an mRNA encoding one or more BH3 domains, optionally wherein the mRNA comprises a regulatory element that decreases expression of the fusion polypeptide in normal cells as compared to cancer cells, and a second mRNA described herein, e.g., an mRNA encoding a prosurvival Bcl-2-like polypeptide or variant or fragment thereof, optionally wherein the second mRNA comprises a regulatory element that decreases expression of the Bcl-2-like polypeptide or variant or fragment thereof in cancer cells as compared to normal cells. In particular embodiments, the regulatory element present in the first mRNA is a binding site for a microRNA that has greater expression in normal cells than cancer cells (e.g., a miR-122 binding site). In particular embodiments, the regulatory element present in the second mRNA is a binding site for a microRNA that has greater expression in cancer cells than normal cells (e.g., a miR-21 binding site), wherein binding of the microRNA to the binding site inhibits expression of the Bcl-2-like polypeptide or variant thereof. In particular embodiments, the subject is provided with a pharmaceutical composition or nanoparticle comprising the first RNA and/or the second RNA. The mRNAs may be taken up and translated in the subject's cells to produce the BH3 domain(s) and the Bcl-2-like polypeptide or variant or fragment thereof. Expression of the one or more BH3 domains is greater in cancer cells than normal cells, resulting in greater apoptosis of cancer cells than normal cells. Expression of the Bcl-2-like polypeptide inhibits apoptosis induced by the fusion polypeptide. Since the Bcl-2-like polypeptide is expressed in higher amounts in normal cells, it inhibits a greater amount of apoptosis induced by the BH3 domain(s) in normal cells as compared to cancer cells.

In certain embodiments, the present invention includes a method of treating or preventing cancer in a subject in need thereof, comprising providing to the subject a first mRNA described herein, e.g., an mRNA encoding one or more BH3 domains, in combination with a therapeutic agent, such as a chemotherapeutic drug or other anti-cancer agent. In certain embodiments, a second mRNA as described herein, e.g., an mRNA encoding a prosurvival Bcl-2-like polypeptide or variant or fragment thereof, is also included in the treatment regimen, together with the first mRNA and the therapeutic agent. Optionally, the second mRNA comprises a regulatory element that decreases expression of the Bcl-2-like polypeptide or variant or fragment thereof in cancer cells as compared to normal cells. Suitable therapeutic agents for use in combination therapy include small molecule chemotherapeutic agents, including protein tyrosine kinase inhibitors, as well as biological anti-cancer agents, such as anti-cancer antibodies. A preferred therapeutic agent for combination therapy is sorafenib. Other suitable therapeutic agents for use in combination therapy are described further below.

A pharmaceutical composition including one or more mRNAs of the invention may be administered to a subject by any suitable route. In some embodiments, compositions of the invention are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. However, the present disclosure encompasses the delivery of compositions of the invention by any appropriate route taking into consideration likely advances in the sciences of drug delivery. In general, the most appropriate route of administration will depend upon a variety of factors including the nature of the pharmaceutical composition including one or more mRNAs (e.g., its stability in various bodily environments such as the bloodstream and gastrointestinal tract), and the condition of the patient (e.g., whether the patient is able to tolerate particular routes of administration).

In certain embodiments, compositions of the invention may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of mRNA or nanoparticle per 1 kg of subject body weight. In particular embodiments, a dose of about 0.005 mg/kg to about 5 mg/kg of mRNA or nanoparticle of the invention may be administrated.

A dose may be administered one or more times per day, in the same or a different amount, to obtain a desired level of mRNA expression and/or effect (e.g., a therapeutic effect). The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition. The specific therapeutically effective, prophylactically effective, or otherwise appropriate dose level for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

In some embodiments, a pharmaceutical composition of the invention may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the invention, or imaging, diagnostic, or prophylactic compositions thereof in combination with agents that improve their bioavailability, reduce and/or modify their metabolism, inhibit their excretion, and/or modify their distribution within the body.

Exemplary therapeutic agents that may be administered in combination with the compositions of the invention include, but are not limited to, cytotoxic, chemotherapeutic, and other therapeutic agents. Cytotoxic agents may include, for example, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, and analogs thereof. Radioactive ions may also be used as therapeutic agents and may include, for example, radioactive iodine, strontium, phosphorous, palladium, cesium, iridium, cobalt, yttrium, samarium, and praseodymium. Other therapeutic agents may include, for example, antimetabolites (e.g., methotrexate, 6-mercaptopurine, 6-thioguanine, cytarabine, and 5-fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, taxol, and maytansinoids).

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

### Methods of Inducing Apoptosis

The invention provides methods of inducing apoptosis in a cell, e.g., a mammalian cell. In some embodiments, a method of inducing apoptosis in a cell involves contacting a cell with an mRNA described herein, e.g., an mRNA encoding one or more BH3 domains. In certain embodiments, such a method involves contacting a cell with an isolated mRNA encoding the one or more intracellular binding domains. In particular embodiments, the cell is contacted with a lipid nanoparticle composition including an mRNA encoding the one or more intracellular binding domains. Upon contacting the cell with the lipid nanoparticle composition or the isolated mRNA, the mRNA may be taken up and translated in the cell to produce one or more domains intracellularly.

The invention further provides methods of selectively inducing apoptosis in a cancer cell as compared to a normal cell. In some embodiments, a method of selectively inducing apoptosis in a cancer cell involves contacting a cell with an mRNA described herein, e.g., an mRNA encoding one or more BH3 domains, wherein the mRNA further comprises a regulatory element that reduces expression of the fusion polypeptide in normal cells as compared to cancer cells. In particular embodiments, the regulatory element is a binding site for a microRNA that has greater expression in normal cells than cancer cells (e.g., a miR-122 binding site), wherein binding of the microRNA to the binding site inhibits expression of the BH3 domain(s). In particular embodiments, the cell is contacted with a nanoparticle composition comprising an mRNA comprising a region encoding the BH3 domain(s) and a microRNA binding site. Upon contacting the cell with the nanoparticle composition or the isolated mRNA, the mRNA may be taken up and translated in the cell to produce the BH3 domain(s). Expression of the BH3 domain(s) is greater in cancer cells than normal cells, resulting in greater apoptosis of cancer cells than normal cells.

In some embodiments, a method of selectively inducing apoptosis in a cancer cell involves contacting a cell with a first mRNA described herein, e.g., an mRNA encoding one or more BH3 domains, optionally wherein the mRNA comprises a regulatory element that reduces expression of the BH3 domain(s) in normal cells as compared to cancer cells, and a second mRNA described herein, e.g., an mRNA encoding a prosurvival Bcl-2-like polypeptide or variant or fragment thereof, optionally wherein the second mRNA comprises a regulatory element that reduces expression of the Bcl-2-like polypeptide or variant thereof in cancer cells as compared to normal cells. In particular embodiments, the regulatory element in the first mRNA is a binding site for a microRNA that has greater expression in normal cells than cancer cells (e.g., a miR-122 binding site), wherein binding of the microRNA to the binding site inhibits expression of the fusion polypeptide. In particular embodiments, the regulatory element in the second mRNA is a binding site for a microRNA that has greater expression in cancer cells than normal cells (e.g., a miR-21 binding site), wherein binding of the microRNA to the binding site inhibits expression of the Bcl-2-like polypeptide or variant thereof. In particular embodiments, the cell is contacted with a nanoparticle composition comprising the first RNA and the second RNA. Upon contacting the cell with the lipid nanoparticle composition or the isolated mRNAs, the mRNAs may be taken up and translated in the cell to produce the BH3 domain(s) and the Bcl-2-like polypeptide or variant thereof. Expression of the BH3 domain(s) is greater in cancer cells than normal cells, resulting in greater apoptosis of cancer cells than normal cells. Expression of the Bcl-2-like polypeptide inhibits apoptosis induced by the BH3 domain(s). Since the Bcl-2-like polypeptide is expressed in higher amounts in normal cells, it inhibits a greater amount of apoptosis induced by the BH3 domain(s) in normal cells as compared to cancer cells. In particular embodiments, a normal cell is a non-cancerous cell of the same cell type, e.g., as the cancer cell.

In general, the step of contacting a mammalian cell with a composition (e.g., an isolated mRNA, nanoparticle, or pharmaceutical composition of the invention) may be performed in vivo, ex vivo, in culture, or in vitro. In exemplary embodiments of the invention, the step of contacting a mammalian cell with a composition (e.g., an isolated mRNA, nanoparticle, or pharmaceutical composition of the invention) is performed in vivo or ex vivo. The amount of the composition contacted with a cell, and/or the amount of mRNA therein, may depend on the type of cell or tissue being contacted, the means of administration, the physiochemical characteristics of the composition and the mRNA (e.g., size, charge, and chemical composition) therein, and other factors. In general, an effective amount of the composition will allow for efficient production of the encoded polypeptide in the cell. Metrics for efficiency may include polypeptide translation (indicated by polypeptide expression), level of mRNA degradation, and immune response indicators.

The step of contacting a composition including an mRNA, or an isolated mRNA, with a cell may involve or cause transfection. In some embodiments, a phospholipid included in a lipid nanoparticle may facilitate transfection and/or increase transfection efficiency, for example, by interacting and/or fusing with a cellular or intracellular membrane. Transfection may allow for the translation of the mRNA within the cell. Translation of one or more intracellular binding domains may induce apoptosis in the cell.

The ability of a composition of the invention (e.g., a lipid nanoparticle or isolated mRNA) to induce apoptosis may be readily determined, for example by comparing the ability of the composition to induce apoptosis as compared to known agents or manipulations that may induce apoptosis, including but not limited to: anti-Fas antibody or Fas ligand, staurosporin, interleukins, Apo3 ligand, and TRAIL. A wide variety of methods of determining whether an agent is capable of inducing apoptosis are known in the art, for example, caspase activation assays (e.g., caspase-3/7 activation assays), stains and dyes (e.g., CELLTOX™, MITOTRACKER® Red, propidium iodide, and YOYO3), cell viability assays, cell morphology, and detection of PARP-1 cleavage, for example, by Western blotting with an anti-PARP-1 antibody.

### Definitions

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Apoptosis: As used herein, “apoptosis” refers to a form of cell death in which a programmed sequence of events leads to the death of a cell. Hallmarks of apoptosis include morphological changes, cell shrinkage, caspase activation, nuclear and cytoplasmic condensation, and alterations in plasma membrane topology. Biochemically, apoptotic cells are characterized by increased intracellular calcium concentration, fragmentation of chromosomal DNA, and expression of novel cell surface components. In particular embodiments, a cell undergoing apoptosis may undergo mitochondrial outer membrane permeabilization (MOMP).

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Bcl-2 homology 3 (BH3) domain: As used herein, a Bcl-2 homology 3 (BH3) domain is a polypeptide or fragment thereof derived from or having homology to a conserved amino acid sequence from a Bcl-2 family protein. In some embodiments, a BH3 domain is capable of inhibiting an anti-apoptotic or prosurvival Bcl-2 family member (including, for example, BCL-2, BCL-X_{L}, BCL-w, MCL-1 and/or BCL2A1 (also referred to as A1)). Bcl-2 family members typically include at least one of four Bcl-2 homology domains (BH1 domain, BH2 domain, BH3 domain, and BH4 domain), which are highly conserved (see, for example, Ankevar et al. Front. Oncol. 1: 34, 2011). There are two main categories of Bcl-2 family proteins: pro-apoptotic Bcl-2 family members and anti-apoptotic Bcl-2 family members. Pro-apoptotic BCL family proteins include “effector” BCL-2 proteins (e.g., BAK and BAX) and “BH3-only” proteins (e.g., BID, BIM, BAD, BAK, BMF, bNIP3, HRK, Noxa, and PUMA). In particular embodiments, the BH3 domain is derived from a BH3-only protein.

Bcl-2-like polypeptide: As used herein, a “Bcl-2-like polypeptide” is a member of the Bcl-2 family of proteins that binds a BH3 peptide and, in certain instances, promotes cell survival. Members of the Bcl-2 protein family that promote cell survival include: Bcl-2, Bcl-X_{L}, Bcl-w, Mcl-1 and A-1. Bcl-2-like polypeptides also include variants of these prosurvival Bcl-2 family members that retain the ability to bind a BH3 domain and/or promote cell survival, which may be termed “functional variants.” “Variants” are polypeptides that include one or more amino acid modifications, e.g., deletions, substitutions or insertions, as compared to a wild-type Bcl-2-like polypeptide. Variants include both fragments of Bcl-2-like polypeptides.

Cancer: As used herein, “cancer” is a condition involving abnormal and/or unregulated cell growth. The term cancer encompasses benign and malignant cancers. Exemplary non-limiting cancers include adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, myelodysplastic syndrome (including refractory anemias and refractory cytopenias), myeloproliferative neoplasms or diseases (including polycythemia vera, essential thrombocytosis and primary myelofibrosis), liver cancer (e.g., hepatocellular carcinoma), non-small cell lung cancer, small cell lung cancer, lung carcinoid tumor, lymphoma of the skin, malignant mesothelioma, multiple myeloma, myelodysplasia syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In particular embodiments, the cancer is liver cancer (e.g., hepatocellular carcinoma) or colorectal cancer. In other embodiments, the cancer is a blood-based cancer or a hematopoetic cancer.

Cleavable Linker: As used herein, the term “cleavable linker” refers to a linker, typically a peptide linker (e.g., about 5-30 amino acids in length, typically about 10-20 amino acids in length) that can be incorporated into multicistronic mRNA constructs such that equimolar levels of multiple genes can be produced from the same mRNA. Non-limiting examples of cleavable linkers include the 2A family of peptides, including F2A, P2A, T2A and E2A, first discovered in picornaviruses, that when incorporated into an mRNA construct (e.g., between two polypeptide domains) function by making the ribosome skip the synthesis of a peptide bond at C-terminus of the 2A element, thereby leading to separation between the end of the 2A sequence and the next peptide downstream.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the invention, the step of contacting a mammalian cell with a composition (e.g., an isolated mRNA, nanoparticle, or pharmaceutical composition of the invention) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle or an isolated mRNA) and a cell may be contacted, for example, by adding the composition to the culture medium of the cell and may involve or result in transfection. Moreover, more than one cell may be contacted by a nanoparticle composition.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the invention may be encapsulated in a lipid nanoparticle, e.g., a liposome.

Effective amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent. In some embodiments, a therapeutically effective amount is an amount of an agent to be delivered (e.g., nucleic acid, drug, therapeutic agent, diagnostic agent or prophylactic agent) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Identity: As used herein, the term “identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux et al., Nucleic Acids Research, 12(1): 387, 1984, BLASTP, BLASTN, and FASTA, Altschul, S. F. et al., J. Molec. Biol., 215, 403, 1990.

Fragment: A “fragment,” as used herein, refers to a portion. For example, fragments of proteins may include polypeptides obtained by digesting full-length protein isolated from cultured cells or obtained through recombinant DNA techniques.

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein.

Hydrophobic amino acid: As used herein, a “hydrophobic amino acid” is an amino acid having an uncharged, nonpolar side chain. Examples of naturally occurring hydrophobic amino acids are alanine (Ala), valine (Val), leucine (Leu), isoleucine (Ile), proline (Pro), phenylalanine (Phe), methionine (Met), and tryptophan (Trp).

Inducing Apoptosis: As used herein, “inducing apoptosis” is meant to indicate a process or method of triggering changes in a cell that, once initiated, will lead to apoptosis. The ability of a substance to induce apoptosis may be readily determined, for example by comparing the ability of a substance to induce apoptosis as compared to known agents or manipulations that may induce apoptosis, including but not limited to: anti-Fas antibody or Fas ligand, staurosporin, interleukins, Apo3 ligand, and TRAIL. A variety of methods of determining whether an agent can induce apoptosis are known in the art, for example, caspase activation assays (e.g., caspase-3/7 activation assays), stains and dyes (e.g., CELLTOX™ MITOTRACKER® Red, propidium iodide, and YOYO3), cell viability assays, cell morphology, and PARP-1 cleavage.

Insertion: As used herein, an “insertion” or an “addition” refers to a change in an amino acid or nucleotide sequence resulting in the addition of one or more amino acid residues or nucleotides, respectively, to a molecule as compared to a reference sequence, for example, the sequence found in a naturally-occurring molecule. For example, an amino acid sequence of a heterologous polypeptide (e.g., a BH3 domain) may be inserted into a scaffold polypeptide (e.g. a SteA scaffold polypeptide) at a site that is amenable to insertion. In some embodiments, an insertion may be a replacement, for example, if an amino acid sequence that forms a loop of a scaffold polypeptide (e.g., loop 1 or loop 2 of SteA or a SteA derivative) is replaced by an amino acid sequence of a heterologous polypeptide.

Insertion Site: As used herein, an “insertion site” is a position or region of a scaffold polypeptide that is amenable to insertion of an amino acid sequence of a heterologous polypeptide. It is to be understood that an insertion site also may refer to the position or region of the polynucleotide that encodes the polypeptide (e.g., a codon of a polynucleotide that codes for a given amino acid in the scaffold polypeptide). In some embodiments, insertion of an amino acid sequence of a heterologous polypeptide into a scaffold polypeptide has little to no effect on the stability (e.g., conformational stability), expression level, or overall secondary structure of the scaffold polypeptide.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Loop 1 insertion site: As used herein, a “loop 1 insertion site” is an insertion site of a SteA scaffold polypeptide in or near loop 1. In some embodiments, a loop 1 insertion site includes one or more of codons 46 to 54 inclusive of a SteA scaffold polypeptide that encode amino acids within or adjacent to loop 1 of a SteA scaffold polypeptide.

Loop 2 insertion site: As used herein, a “loop 2 insertion site” is an insertion site of a SteA scaffold polypeptide in or near loop 2. In some embodiments, a loop 2 insertion site includes one or more of codons 67 to 84 inclusive of a SteA scaffold polypeptide that encode amino acids within or adjacent to loop 2 of a SteA scaffold polypeptide.

Metastasis: As used herein, the term “metastasis” means the process by which cancer spreads from the place at which it first arose as a primary tumor to distant locations in the body. A secondary tumor that arose as a result of this process may be referred to as “a metastasis.” mRNA: As used herein, an “mRNA” refers to a messenger ribonucleic acid.

An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

microRNA (miRNA): As used herein, a “microRNA (miRNA)” is a small noncoding RNA molecule which may function in post-transcriptional regulation of gene expression (e.g., by RNA silencing, such as by cleavage of the mRNA, destabilization of the mRNA by shortening its polyA tail, and/or by interfering with the efficiency of translation of the mRNA into a polypeptide by a ribosome). A mature miRNA is typically about 22 nucleotides long.

microRNA-122 (miR-122): As used herein, “microRNA-122 (miR-122)” refers to any native miR-122 from any vertebrate source, including, for example, humans, unless otherwise indicated. miR-122 is typically highly expressed in the liver, where it may regulate fatty-acid metabolism. miR-122 levels are reduced in liver cancer, for example, hepatocellular carcinoma. miR-122 is one of the most highly-expressed miRNAs in the liver, where it regulates targets including but not limited to CAT-1, CD320, AldoA, Hjv, Hfe, ADAM10, IGFR1, CCNG1, and ADAM17. Mature human miR-122 may have a sequence of AACGCCAUUAUCACACUAAAUA (SEQ ID NO: 31, corresponding to hsa-miR-122-3p) or UGGAGUGUGACAAUGGUGUUUG (SEQ ID NO: 32, corresponding to hsa-miR-122-5p).

microRNA-21 (miR-21): As used herein, “microRNA-21 (miR-21)” refers to any native miR-21 from any vertebrate source, including, for example, humans, unless otherwise indicated. miR-21 levels are increased in liver cancer, for example, hepatocellular carcinoma, as compared to normal liver. Mature human miR-21 may have a sequence of UAGCUUAUCAGACUGAUGUUGA (SEQ ID NO: 33, corresponding to has-miR-21-5p) or 5′-CAACACCAGUCGAUGGGCUGU-3′ (SEQ ID NO: 34, corresponding to has-miR-21-3p). An additional miR-21 sequence is shown in SEQ ID NO: 106.

microRNA-142 (miR-142): As used herein, “microRNA-142 (miR-142)” refers to any native miR-142 from any vertebrate source, including, for example, humans, unless otherwise indicated. miR-142 is typically highly expressed in myeloid cells. Mature human miR-142 may have a sequence of UGUAGUGUUUCCUACUUUAUGGA (SEQ ID NO: 295, corresponding to hsa-miR-142-3p) or CAUAAAGUAGAAAGCACUACU (SEQ ID NO: 296, corresponding to hsa-miR-142-5p).

microRNA (miRNA) binding site: As used herein, a “microRNA (miRNA) binding site” refers to a miRNA target site or a miRNA recognition site, or any nucleotide sequence to which a miRNA binds or associates. In some embodiments, a miRNA binding site represents a nucleotide location or region of a polynucleotide (e.g., an mRNA) to which at least the “seed” region of a miRNA binds. It should be understood that “binding” may follow traditional Watson-Crick hybridization rules or may reflect any stable association of the miRNA with the target sequence at or adjacent to the microRNA site.

miRNA seed: As used herein, a “seed” region of a miRNA refers to a sequence in the region of positions 2-8 of a mature miRNA, which typically has perfect Watson-Crick complementarity to the miRNA binding site. A miRNA seed may include positions 2-8 or 2-7 of a mature miRNA. In some embodiments, a miRNA seed may comprise 7 nucleotides (e.g., nucleotides 2-8 of a mature miRNA), wherein the seed-complementary site in the corresponding miRNA binding site is flanked by an adenine (A) opposed to miRNA position 1. In some embodiments, a miRNA seed may comprise 6 nucleotides (e.g., nucleotides 2-7 of a mature miRNA), wherein the seed-complementary site in the corresponding miRNA binding site is flanked by an adenine (A) opposed to miRNA position 1. When referring to a miRNA binding site, an miRNA seed sequence is to be understood as having complementarity (e.g., partial, substantial, or complete complementarity) with the seed sequence of the miRNA that binds to the miRNA binding site.

Modified: As used herein “modified” refers to a changed state or structure of a molecule of the invention. Molecules may be modified in many ways including chemically, structurally, and functionally. In one embodiment, the mRNA molecules of the present invention are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 mn. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

N-terminal insertion site: As used herein, an “N-terminal insertion site” is an insertion site of a SteA scaffold polypeptide in the N-terminus. In some embodiments, an N-terminal insertion site includes one or more of the first 8 codons encoding the first 8 amino acids of a SteA scaffold polypeptide. In particular embodiments, an N-terminal insertion site is position 4 of a SteA scaffold polypeptide.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the invention include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Stefin A (SteA) scaffold polypeptide: By “Stefin A (SteA) scaffold polypeptide” is meant a SteA polypeptide or any derivative thereof able to accept an insertion of a heterologous polypeptide amino acid sequence. In some embodiments, a SteA scaffold polypeptide includes an N-terminal insertion site, a loop 1 insertion site, and/or a loop 2 insertion site. Exemplary SteA scaffold polypeptides include wild-type Stefin A (for example, human Stefin A, also known as cystatin A), STM (Stefin A Triple Mutant, as described, e.g., in U.S. Pat. No. 8,063,019, incorporated herein by reference, and in Woodman et al., J. Mol. Biol. 352: 1118-1133, 2005), SQM (Stefin A Quadruple Mutant, as described, e.g., in U.S. Pat. No. 8,853,131, incorporated herein by reference), and SQT (Stefin A Quadruple Mutant, Tracy, as described, e.g., in U.S. Pat. No. 8,853,131, incorporated herein by reference and Stadler et al., Protein Eng. Des. Sel. 24(9): 751-763, 2011). Additional exemplary SteA scaffold proteins are described in U.S. Pat. No. 8,853,131, incorporated herein by reference, including SDM, SUC, SUM, SUN, SDM-, SDM--, SQM-, SQM--, SUC-, SUC--, SUM-, SUM--, SUN-, SUN--, and SQL.

Subject: As used herein, the term “subject” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as a mRNA) into a cell.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Tumor: As used herein, a “tumor” is an abnormal growth of tissue, whether benign or malignant.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in an sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in an sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence.

Yes-associated protein (YAP): As used herein, Yes-associated protein (YAP) is a member of the Hippo pathway. In certain embodiments, YAP includes YAP1. When the Hippo pathway is “on”, mammalian STE20-like protein kinase 1 (MST1) or MST2 phosphorylate Salvador homolog1 (SAV1), and together they phosphorylate and activate MOB kinase activator 1A (MOB1A), MOB1B, large tumor suppressor homolog 1 (LATS1) kinase and LATS2 kinase, which then phosphorylate YAP and transcriptional co-activator with PDZ-binding motif (TAZ). Phosphorylated YAP and TAZ are sequestered in the cytoplasm by the 14-3-3 protein and shunted for proteasomal degradation. As a result, the TEA domain-containing sequence-specific transcription factors (TEADs) associate with the transcription co-factor vestigial-like protein 4 (VGLL4) and suppress target gene expression. When the Hippo pathway is “off”, the kinases MST1, MST2, LATS1 and LATS2 are inactive, therefore YAP and TAZ accumulate in the nucleus where they displace VGLL4 and form a complex with TEADs, thereby promoting the expression of target genes. (see e.g., Meng et al., Genes & Dev. Vol. 30: 1-17, 2016)

YAP binding polypeptide: As used herein, a Yes-associated protein (YAP) binding polypeptide, used interchangeably with “YAP inhibitory domain”, is a polypeptide or fragment thereof derived from or having homology to a conserved amino acid sequence from a Vestigial-like protein (VGLL) family member (including, for example, VGLL1, VGLL2, VGLL3, and/or VGLL4). In some embodiments, a YAP inhibitory domain is capable of displacing YAP from the TEADs and inhibiting expression of target genes.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Preparation and Expression of mRNAs Encoding Scaffold-Less BH3 Domains

A series of modified mRNA constructs were prepared that encoded one or more copies of selected BH3 domains from the “BH3 only” family of pro-apoptotic BCL-2 family proteins (PUMA, Bim, Bad and Noxa) in the absence of any “scaffold” polypeptide. Constructs containing multiple BH3 domains also encoded a linker sequence (e.g., an F2A or P2A cleavable linker) between each BH3 domain. Thus, for example, a multimeric BH3 construct containing three BH3 domains with intervening F2A linkers has the structure: BH3-F2A-BH3-F2A-BH3. The constructs also typically contained an epitope tag (e.g., FLAG, His, v5) to facilitate detection of the encoded polypeptide(s). Some multimeric BH3 constructs contained F2A linkers between each BH3 domain and before the C-terminal epitope tag (e.g., having the structure: BH3-F2A-BH3-F2A-BH3-F2A-affinity tag). Certain constructs also included one or more miR binding sites (e.g., miR122 binding site, miR142.3p binding site). Certain construct encoded only a single BH3 domain, while other “multimer” constructs encoded three, five or ten BH3 domains. In certain multimer constructs, multiple copies of the same BH3 domain were used. In other multimer constructs, different BH3 domains were used in combination to achieve the multiple copies of BH3 domains in the construct. The amino acid sequence encoded by the open reading frame (ORF) of each construct is shown below in Table 15:

TABLE 15

| | | SEQ |
|---|---|---|
| | | ID |
| mRNA Name(s) | ORF Amino Acid Sequence | NO |
| PumaBH3_HS3UPCRfree | MEEQWAREIGAQLRRMADDLNAQYERR | 142 |
| PumaBH3.nHIS_HS3UPCRfree | MHHHHHHEEQWAREIGAQLRRMADDL | 143 |
| | NAQYERR | |
| PumaBH3.nV5_HS3UPCRfree | MGKPIPNPLLGLDSTEEQWAREIGAQLRR | 144 |
| | MADDLNAQYERR | |
| PumaBH3.cHIS_HS3UPCRfree | MEEQWAREIGAQLRRMADDLNAQYERR | 145 |
| | HHHHHH | |
| PumaBH3(x3.F2A).v5_ | MEEQWAREIGAQLRRMADDLNAQYERR | 146 |
| Hs3UPCRfree | GSGVKQTLNFDLLKLAGDVESNPGPEEQW | |
| | AREIGAQLRRMADDLNAQYERRGSGVKQT | |
| | LNFDLLKLAGDVESNPGPEEQWAREIGAQL | |
| | RRMADDLNAQYERRGKPIPNPLLGLDST | |
| PumaBH3.cV5_HS3UPCRfree | MEEQWAREIGAQLRRMADDLNAQYERR | 147 |
| | GKPIPNPLLGLDST | |
| PumaBH3(x3.F2A).v5_miR142.3p | MEEQWAREIGAQLRRMADDLNAQYERRG | 148 |
| _tp | SGVKQTLNFDLLKLAGDVESNPGPEEQWAR | |
| | EIGAQLRRMADDLNAQYERRGSGVKQTLNF | |
| | DLLKLAGDVESNPGPEEQWAREIGAQLRRM | |
| | ADDLNAQYERRGKPIPNPLLGLDST | |
| PumaBH3(x3.F2A).v5_miR122/1 | MEEQWAREIGAQLRRMADDLNAQYERRGS | 149 |
| 42.3p_tp | GVKQTLNFDLLKLAGDVESNPGPEEQWAREI | |
| | GAQLRRMADDLNAQYERRGSGVKQTLNFDL | |
| | LKLAGDVESNPGPEEQWAREIGAQLRRMAD | |
| | DLNAQYERRGKPIPNPLLGLDST | |
| PumaBH3(x3.F2A).v5_tp | MEEQWAREIGAQLRRMADDLNAQYERRGS | 150 |
| | GVKQTLNFDLLKLAGDVESNPGPEEQWAREI | |
| | GAQLRRMADDLNAQYERRGSGVKQTLNFDL | |
| | LKLAGDVESNPGPEEQWAREIGAQLRRMAD | |
| | DLNAQYERRGKPIPNPLLGLDST | |
| PumaBH3(x3.F2A).v5_miR122_ | MEEQWAREIGAQLRRMADDLNAQYERRGS | 151 |
| tp | GVKQTLNFDLLKLAGDVESNPGPEEQWAREI | |
| | GAQLRRMADDLNAQYERRGSGVKQTLNFDL | |
| | LKLAGDVESNPGPEEQWAREIGAQLRRMAD | |
| | DLNAQYERRGKPIPNPLLGLDST | |
| (pumaBH3.F2A)\hat{ }5_cV5, | MEEQWAREIGAQLRRMADDLNAQYERRGS | 152 |
| (pumaBH3.F2A)\hat{ }5_cV5_DX | GVKQTLNFDLLKLAGDVESNPGPEEQWAREI | |
| | GAQLRRMADDLNAQYERRGSGVKQTLNFDL | |
| | KAGDVESNPGPEEQWAREIGAQLRRMADDN | |
| | AQYERRGSGVKQTLNFDLLKLAGDVESNPGP | |
| | EEQWAREIGAQLRRMADDLNAQYERRGSGV | |
| | KQTLNFDLLKLAGDVESNPGPEEQWAREIGA | |
| | QLRRMADDLNAQYERRGSGVKQTLNFDLLK | |
| | LAGDVESNPGPGKPIPNPLLGLDST | |
| (BimBH3.F2A)\hat{ }5_cV5, | MDMRPEIWIAQELRRIGDEFNAYYARRGSGV | 153 |
| (BimBH3.F2A)\hat{ }5_cV5_DX | KQTLNFDLLKLAGDVESNPGPDMRPEIWIAQ | |
| | ELRRIGDEFNAYYARRGSGVKQTLNFDLLKLA | |
| | GDVESNPGPDMRPEIWIAQELRRIGDEFNAY | |
| | YARRGSGVKQTLNFDLLKLAGDVESNPGPDM | |
| | RPEIWIAQELRRIGDEFNAYYARRGSGVKQTL | |
| | NFDLLKLAGDVESNPGPDMRPEIWIAQELRRI | |
| | GDEFNAYYARRGSGVKQTLNFDLLKLAGDVE | |
| | SNPGPGKPIPNPLLGLDST | |
| (SuperPumaBH3.F2A)\hat{ }3_cV5 | MQWAREIGAQLRRIGDDLNAQYERRRQGSG | 154 |
| | VKQTLNFDLLKLAGDVESNPGPQWAREIGAQ | |
| | LRRIGDDLNAQYERRRQGSGVKQTLNFDLLKL | |
| | AGDVESNPGPQWAREIGAQLRRIGDDLNAQ | |
| | YERRRQGSGVKQTLNFDLLKLAGDVESNPGP | |
| | GSGVKQTLNFDLLKLAGDVESNPGPGKPIPNP | |
| | LLGLDST | |
| (SuperPumaBH3.F2A)\hat{ }5_cV5 | MQWAREIGAQLRRIGDDLNAQYERRRQGSV | 155 |
| | KQTLNFDLLKLAGDVESNPGPQWAREIGAQR | |
| | RIGDDLNAQYERRRQGSGVKQTLNFDLLKLAG | |
| | DVESNPGPQWAREIGAQLRRIGDDLNAQYER | |
| | RRQGSGVKQTLNFDLLKLAGDVESNPGPQA | |
| | REIGAQLRRIGDDLNAQYERRRQGSGVKQTN | |
| | FDLLKLAGDVESNPGPQWAREIGAQLRRIGD | |
| | LNAQYERRRQGSGVKQTLNFDLLKLAGDVEN | |
| | PGPGKPIPNPLLGLDST | |
| (KittyCatBH3.F2A)\hat{ }5_cV5, | MQWAREIGAQERREADDENAQYERRRQGS | 156 |
| (KittyCatBH3.F2A)\hat{ }5_cV5_DX | GVKQTLNFDLLKLAGDVESNPGPQWAREIGA | |
| | QERREADDENAQYERRRQGSGVKQTLNFDLL | |
| | KLAGDVESNPGPQWAREIGAQERREADDENA | |
| | QYERRRQGSGVKQTLNFDLLKLAGDVESNPGP | |
| | QWAREIGAQERREADDENAQYERRRQGSGVK | |
| | QTLNFDLLKLAGDVESNPGPQWAREIGAQERR | |
| | EADDENAQYERRRQGSGVKQTLNFDLLKLAGD | |
| | VESNPGPGKPIPNPLLGLDST | |
| (KittyCatBH3.F2A)\hat{ }5_cV5, | MQWAREIGAQERREADDENAQYERRRQGSG | 157 |
| (KittyCatBH3.F2A)\hat{ }5_cV5_DX | VKQTLNFDLLKLAGDVESNPGPQWAREIGAQ | |
| | ERREADDENAQYERRRQGSGVKQTLNFDLLKL | |
| | AGDVESNPGPQWAREIGAQERREADDENAQY | |
| | ERRRQGSGVKQTLNFDLLKLAGDVESNPGPQW | |
| | AREIGAQERREADDENAQYERRRQGSGVKQTL | |
| | NFDLLKLAGDVESNPGPQWAREIGAQERREAD | |
| | DENAQYERRRQGSGVKQTLNFDLLKLAGDVES | |
| | NPGPGKPIPNPLLGLDST | |
| (BadBH3.F2A)\hat{ }5_cV5 | MNLWAAQRYGRELRRMSDEFVDSFKKGGSG | 158 |
| | VKQTLNFDLLKLAGDVESNPGPNLWAAQRYG | |
| | RELRRMSDEFVDSFKKGGSGVKQTLNFDLLKLA | |
| | GDVESNPGPNLWAAQRYGRELRRMSDEFVDS | |
| | FKKGGSGVKQTLNFDLLKLAGDVESNPGPNLW | |
| | AAQRYGRELRRMSDEFVDSFKKGGSGVKQTL | |
| | NFDLLKLAGDVESNPGPNLWAAQRYGRELRR | |
| | MSDEFVDSFKKGGSGVKQTLNFDLLKLAGDV | |
| | ESNPGPGKPIPNPLLGLDST | |
| (Noxa-F2A-BadBH3-F2A)\hat{ }3_cV5 | MPAELEVECATQLRRFGDKLNFRQKLLGSGVK | 159 |
| | QTLNFDLLKLAGDVESNPGPNLWAAQRYGREL | |
| | RRMSDEFVDSFKKGGSGVKQTLNFDLLKLAGD | |
| | VESNPGPPAELEVECATQLRRFGDKLNFRQKLL | |
| | GSGVKQTLNFDLLKLAGDVESNPGPNLWAAQ | |
| | RYGRELRRMSDEFVDSFKKGGSGVKQTLNFDL | |
| | LKLAGDVESNPGPPAELEVECATQLRRFGDKLN | |
| | FRQKLLGSGVKQTLNFDLLKLAGDVESNPGPNL | |
| | WAAQRYGRELRRMSDEFVDSFKKGGSGVKQT | |
| | LNFDLLKLAGDVESNPGPGKPIPNPLLGLDST | |
| (pumaBH3.F2A)\hat{ }10_cV5, | MEEQWAREIGAQLRRMADDLNAQYERRGSG | 160 |
| (pumaBH3.F2A)\hat{ }10_cV5_DX | VKQTLNFDLLKLAGDVESNPGPEEQWAREIGA | |
| | QLRRMADDLNAQYERRGSGVKQTLNFDLLKLA | |
| | GDVESNPGPEEQWAREIGAQLRRMADDLNAQ | |
| | YERRGSGVKQTLNFDLLKLAGDVESNPGPEEQW | |
| | AREIGAQLRRMADDLNAQYERRGSGVKQTLNF | |
| | DLLKLAGDVESNPGPEEQWAREIGAQLRRMAD | |
| | DLNAQYERRGSGVKQTLNFDLLKLAGDVESNPG | |
| | PEEQWAREIGAQLRRMADDLNAQYERRGSGVK | |
| | QTLNFDLLKLAGDVESNPGPEEQWAREIGAQLR | |
| | RMADDLNAQYERRGSGVKQTLNFDLLKLAGDVE | |
| | SNPGPEEQWAREIGAQLRRMADDLNAQYERRG | |
| | SGVKQTLNFDLLKLAGDVESNPGPEEQWAREIGA | |
| | QLRRMADDLNAQYERRGSGVKQTLNFDLLKLAG | |
| | DVESNPGPEEQWAREIGAQLRRMADDLNAQYE | |
| | RRGSGVKQTLNFDLLKLAGDVESNPGPGKPIPNP | |
| | LLGLDST | |
| (BimBH3.F2A)\hat{ }10_cV5, | MDMRPEIWIAQELRRIGDEFNAYYARRGSGVK | 161 |
| (BimBH3.F2A)\hat{ }10_cV5_DX | QTLNFDLLKLAGDVESNPGPDMRPEIWIAQELR | |
| | RIGDEFNAYYARRGSGVKQTLNFDLLKLAGDVES | |
| | NPGPDMRPEIWIAQELRRIGDEFNAYYARRGSG | |
| | VKQTLNFDLLKLAGDVESNPGPDMRPEIWIAQE | |
| | LRRIGDEFNAYYARRGSGVKQTLNFDLLKLAGDV | |
| | ESNPGPDMRPEIWIAQELRRIGDEFNAYYARRG | |
| | SGVKQTLNFDLLKLAGDVESNPGPDMRPEIWIA | |
| | QELRRIGDEFNAYYARRGSGVKQTLNFDLLKLAG | |
| | DVESNPGPDMRPEIWIAQELRRIGDEFNAYYAR | |
| | RGSGVKQTLNFDLLKLAGDVESNPGPDMRPEIW | |
| | IAQELRRIGDEFNAYYARRGSGVKQTLNFDLLKLA | |
| | GDVESNPGPDMRPEIWIAQELRRIGDEFNAYYAR | |
| | RGSGVKQTLNFDLLKLAGDVESNPGPDMRPEIW | |
| | IAQELRRIGDEFNAYYARRGSGVKQTLNFDLLKLA | |
| | GDVESNPGPGKPIPNPLLGLDST | |
| (Puma-P2A-BimBH3-F2A)\hat{ }3_cV5 | MEEQWAREIGAQLRRMADDLNAQYERRGSGVK | 162 |
| | QTLNFDLLKLAGDVESNPGPDMRPEIWIAQELRR | |
| | IGDEFNAYYARRGSGVKQTLNFDLLKLAGDVESN | |
| | PGPEEQWAREIGAQLRRMADDLNAQYERRGSG | |
| | VKQTLNFDLLKLAGDVESNPGPDMRPEIWIAQEL | |
| | RRIGDEFNAYYARRGSGVKQTLNFDLLKLAGDVE | |
| | SNPGPEEQWAREIGAQLRRMADDLNAQYERRG | |
| | SGVKQTLNFDLLKLAGDVESNPGPDMRPEIWIAQ | |
| | ELRRIGDEFNAYYARRGSGVKQTLNFDLLKLAGDV | |
| | ESNPGPGKPIPNPLLGLDST | |
| (Puma-F2A-BimBH3- | MEEQWAREIGAQLRRMADDLNAQYERRGSGVK | 163 |
| F2A)\hat{ }5_cV5, (Puma-F2A- | QTLNFDLLKLAGDVESNPGPDMRPEIWIAQELRR | |
| BimBH3F2A)\hat{ }5_cV5_DX | IGDEFNAYYARRGSGVKQTLNFDLLKLAGDVESN | |
| | PGPEEQWAREIGAQLRRMADDLNAQYERRGSG | |
| | VKQTLNFDLLKLAGDVESNPGPDMRPEIWIAQEL | |
| | RRIGDEFNAYYARRGSGVKQTLNFDLLKLAGDVE | |
| | SNPGPEEQWAREIGAQLRRMADDLNAQYERRG | |
| | SGVKQTLNFDLLKLAGDVESNPGPDMRPEIWIAQ | |
| | ELRRIGDEFNAYYARRGSGVKQTLNFDLLKLAGDV | |
| | ESNPGPEEQWAREIGAQLRRMADDLNAQYERR | |
| | GSGVKQTLNFDLLKLAGDVESNPGPDMRPEIWIA | |
| | QELRRIGDEFNAYYARRGSGVKQTLNFDLLKLAG | |
| | DVESNPGPEEQWAREIGAQLRRMADDLNAQYE | |
| | RRGSGVKQTLNFDLLKLAGDVESNPGPDMRPEI | |
| | WIAQELRRIGDEFNAYYARRGSGVKQTLNFDLLKL | |
| | AGDVESNPGPGKPIPNPLLGLDST | |
| (Puma-F2A-BimBH3- | MEEQWAREIGAQLRRMADDLNAQYERRGSGV | 164 |
| F2A)\hat{ }10_cV5 | KQTLNFDLLKLAGDVESNPGPDMRPEIWIAQEL | |
| | RRIGDEFNAYYARRGSGVKQTLNFDLLKLAGDVE | |
| | SNPGPEEQWAREIGAQLRRMADDLNAQYERRG | |
| | SGVKQTLNFDLLKLAGDVESNPGPDMRPEIWIA | |
| | QELRRIGDEFNAYYARRGSGVKQTLNFDLLKLAG | |
| | DVESNPGPEEQWAREIGAQLRRMADDLNAQYE | |
| | RRGSGVKQTLNFDLLKLAGDVESNPGPDMRPEI | |
| | WIAQELRRIGDEFNAYYARRGSGVKQTLNFDLLK | |
| | LAGDVESNPGPEEQWAREIGAQLRRMADDLNA | |
| | QYERRGSGVKQTLNFDLLKLAGDVESNPGPDMR | |
| | PEIWIAQELRRIGDEFNAYYARRGSGVKQTLNFDL | |
| | LKLAGDVESNPGPEEQWAREIGAQLRRMADDLN | |
| | AQYERRGSGVKQTLNFDLLKLAGDVESNPGPDM | |
| | RPEIWIAQELRRIGDEFNAYYARRGSGVKQTLNF | |
| | DLLKLAGDVESNPGPEEQWAREIGAQLRRMAD | |
| | DLNAQYERRGSGVKQTLNFDLLKLAGDVESNPG | |
| | PDMRPEIWIAQELRRIGDEFNAYYARRGSGVKQT | |
| | LNFDLLKLAGDVESNPGPEEQWAREIGAQLRRM | |
| | ADDLNAQYERRGSGVKQTLNFDLLKLAGDVESN | |
| | PGPDMRPEIWIAQELRRIGDEFNAYYARRGSGV | |
| | KQTLNFDLLKLAGDVESNPGPEEQWAREIGAQL | |
| | RRMADDLNAQYERRGSGVKQTLNFDLLKLAGD | |
| | VESNPGPDMRPEIWIAQELRRIGDEFNAYYARR | |
| | GSGVKQTLNFDLLKLAGDVESNPGPEEQWAREI | |
| | GAQLRRMADDLNAQYERRGSGVKQTLNFDLLK | |
| | LAGDVESNPGPDMRPEIWIAQELRRIGDEFNAY | |
| | YARRGSGVKQTLNFDLLKLAGDVESNPGPEEQW | |
| | AREIGAQLRRMADDLNAQYERRGSGVKQTLNF | |
| | DLLKLAGDVESNPGPDMRPEIWIAQELRRIGDE | |
| | FNAYYARRGSGVKQTLNFDLLKLAGDVESNPGP | |
| | GKPIPNPLLGLDST | |
| (SuperPumaBH3.F2A)\hat{ }10_cV5, | MQWAREIGAQLRRIGDDLNAQYERRRQGSGVK | 165 |
| (SuperPumaBH3.F2A)\hat{ }10_cV5_ | QTLNFDLLKLAGDVESNPGPQWAREIGAQLRRIG | |
| DX | DDLNAQYERRRQGSGVKQTLNFDLLKLAGDVES | |
| | NPGPQWAREIGAQLRRIGDDLNAQYERRRQGS | |
| | GVKQTLNFDLLKLAGDVESNPGPQWAREIGAQ | |
| | LRRIGDDLNAQYERRRQGSGVKQTLNFDLLKLA | |
| | GDVESNPGPQWAREIGAQLRRIGDDLNAQYER | |
| | RRQGSGVKQTLNFDLLKLAGDVESNPGPCIWAR | |
| | EIGAQLRRIGDDLNAQYERRRQGSGVKQTLNFD | |
| | LLKLAGDVESNPGPQWAREIGAQLRRIGDDLNA | |
| | QYERRRQGSGVKQTLNFDLLKLAGDVESNPGPQ | |
| | WAREIGAQLRRIGDDLNAQYERRRQGSGVKQTL | |
| | NFDLLKLAGDVESNPGPQWAREIGAQLRRIGDD | |
| | LNAQYERRRQGSGVKQTLNFDLLKLAGDVESNP | |
| | GPQWAREIGAQLRRIGDDLNAQYERRRQGSGV | |
| | KQTLNFDLLKLAGDVESNPGPGKPIPNPLLGLDST | |
| (KittyCatBH3.F2A)\hat{ }10_cV5, | MQWAREIGAQERREADDENAQYERRRQGSGV | 166 |
| (KittyCatBH3.F2A)\hat{ }10_cV5_DX | KQTLNFDLLKLAGDVESNPGPQWAREIGAQERR | |
| | EADDENAQYERRRQGSGVKQTLNFDLLKLAGDV | |
| | ESNPGPQWAREIGAQERREADDENAQYERRRQ | |
| | GSGVKQTLNFDLLKLAGDVESNPGPQWAREIGA | |
| | QERREADDENAQYERRRQGSGVKQTLNFDLLKL | |
| | AGDVESNPGPQWAREIGAQERREADDENAQYE | |
| | RRRQGSGVKQTLNFDLLKLAGDVESNPGPCIWA | |
| | REIGAQERREADDENAQYERRRQGSGVKQTLNF | |
| | DLLKLAGDVESNPGPQWAREIGAQERREADDEN | |
| | AQYERRRQGSGVKQTLNFDLLKLAGDVESNPGP | |
| | QWAREIGAQERREADDENAQYERRRQGSGVKQ | |
| | TLNFDLLKLAGDVESNPGPQWAREIGAQERREA | |
| | DDENAQYERRRQGSGVKQTLNFDLLKLAGDVES | |
| | NPGPQWAREIGAQERREADDENAQYERRRQGS | |
| | GVKQTLNFDLLKLAGDVESNPGPQWAREIGAQE | |
| | RREADDENAQYERRRQGSGVKQTLNFDLLKLAG | |
| | DVESNPGPGKPIPNPLLGLDST | |
| (KittyCatBH3.F2A)\hat{ }10_cV5, | MQWAREIGAQERREADDENAQYERRRQGSGV | 167 |
| (KittyCatBH3.F2A)\hat{ }10_cV5_DX | KQTLNFDLLKLAGDVESNPGPQWAREIGAQERR | |
| | EADDENAQYERRRQGSGVKQTLNFDLLKLAGDV | |
| | ESNPGPQWAREIGAQERREADDENAQYERRRQ | |
| | GSGVKQTLNFDLLKLAGDVESNPGPQWAREIGA | |
| | QERREADDENAQYERRRQGSGVKQTLNFDLLKL | |
| | AGDVESNPGPQWAREIGAQERREADDENAQYE | |
| | RRRQGSGVKQTLNFDLLKLAGDVESNPGPCIWA | |
| | REIGAQERREADDENAQYERRRQGSGVKQTLNF | |
| | DLLKLAGDVESNPGPQWAREIGAQERREADDE | |
| | NAQYERRRQGSGVKQTLNFDLLKLAGDVESNPG | |
| | PQWAREIGAQERREADDENAQYERRRQGSGVK | |
| | QTLNFDLLKLAGDVESNPGPQWAREIGAQERRE | |
| | ADDENAQYERRRQGSGVKQTLNFDLLKLAGDVE | |
| | SNPGPQWAREIGAQERREADDENAQYERRRQG | |
| | SGVKQTLNFDLLKLAGDVESNPGPQWAREIGAQ | |
| | ERREADDENAQYERRRQGSGVKQTLNFDLLKLA | |
| | GDVESNPGPGKPIPNPLLGLDST | |
| (NoxaBH3.F2A)\hat{ }5_cV5, | MPAELEVECATQLRRFGDKLNFRQKLLGSGVKQT | 168 |
| (NoxaBH3.F2A)\hat{ }5_cV5_DX | LNFDLLKLAGDVESNPGPPAELEVECATQLRRFG | |
| | DKLNFRQKLLGSGVKQTLNFDLLKLAGDVESNPG | |
| | PPAELEVECATQLRRFGDKLNFRQKLLGSGVKQTL | |
| | NFDLLKLAGDVESNPGPPAELEVECATQLRRFGD | |
| | KLNFRQKLLGSGVKQTLNFDLLKLAGDVESNPGP | |
| | PAELEVECATQLRRFGDKLNFRQKLLGSGVKQTL | |
| | NFDLLKLAGDVESNPGPPAELEVECATQLRRFGD | |
| | KLNFRQKLLGSGVKQTLNFDLLKLAGDVESNPGP | |
| | GKPIPNPLLGLDST | |
| (NoxaBH3.F2A)\hat{ }10_cV5, | MPAELEVECATQLRRFGDKLNFRQKLLGSGVKQT | 169 |
| (NoxaBH3.F2A)\hat{ }10_cV5_DX | LNFDLLKLAGDVESNPGPPAELEVECATQLRRFG | |
| | DKLNFRQKLLGSGVKQTLNFDLLKLAGDVESNPG | |
| | PPAELEVECATQLRRFGDKLNFRQKLLGSGVKQTL | |
| | NFDLLKLAGDVESNPGPPAELEVECATQLRRFGD | |
| | KLNFRQKLLGSGVKQTLNFDLLKLAGDVESNPGP | |
| | PAELEVECATQLRRFGDKLNFRQKLLGSGVKQTL | |
| | NFDLLKLAGDVESNPGPPAELEVECATQLRRFGD | |
| | KLNFRQKLLGSGVKQTLNFDLLKLAGDVESNPGP | |
| | PAELEVECATQLRRFGDKLNFRQKLLGSGVKQTL | |
| | NFDLLKLAGDVESNPGPPAELEVECATQLRRFGD | |
| | KLNFRQKLLGSGVKQTLNFDLLKLAGDVESNPGP | |
| | PAELEVECATQLRRFGDKLNFRQKLLGSGVKQTL | |
| | NFDLLKLAGDVESNPGPPAELEVECATQLRRFGD | |
| | KLNFRQKLLGSGVKQTLNFDLLKLAGDVESNPGP | |
| | PAELEVECATQLRRFGDKLNFRQKLLGSGVKQTL | |
| | NFDLLKLAGDVESNPGPGKPIPNPLLGLDST | |
| (BadBH3.F2A)\hat{ }10_cV5, | MNLWAAQRYGRELRRMSDEFVDSFKKGGSGVK | 170 |
| (BadBH3.F2A)\hat{ }10_cV5_DX | QTLNFDLLKLAGDVESNPGPNLWAAQRYGRELR | |
| | RMSDEFVDSFKKGGSGVKQTLNFDLLKLAGDVES | |
| | NPGPNLWAAQRYGRELRRMSDEFVDSFKKGGS | |
| | GVKQTLNFDLLKLAGDVESNPGPNLWAAQRYGR | |
| | ELRRMSDEFVDSFKKGGSGVKQTLNFDLLKLAGD | |
| | VESNPGPNLWAAQRYGRELRRMSDEFVDSFKKG | |
| | GSGVKQTLNFDLLKLAGDVESNPGPNLWAAQRY | |
| | GRELRRMSDEFVDSFKKGGSGVKQTLNFDLLKLA | |
| | GDVESNPGPNLWAAQRYGRELRRMSDEFVDSFK | |
| | KGGSGVKQTLNFDLLKLAGDVESNPGPNLWAAQ | |
| | RYGRELRRMSDEFVDSFKKGGSGVKQTLNFDLLK | |
| | LAGDVESNPGPNLWAAQRYGRELRRMSDEFVDS | |
| | FKKGGSGVKQTLNFDLLKLAGDVESNPGPNLWA | |
| | AQRYGRELRRMSDEFVDSFKKGGSGVKQTLNFD | |
| | LLKLAGDVESNPGPGKPIPNPLLGLDST | |
| (Noxa-F2A-BadBH3-F2A)\hat{ }5_cV5, | MPAELEVECATQLRRFGDKLNFRQKLLGSGVKQT | 171 |
| (Noxa-F2A-BadBH3- | LNFDLLKLAGDVESNPGPNLWAAQRYGRELRRM | |
| F2A)\hat{ }5_cV5_DX | SDEFVDSFKKGGSGVKQTLNFDLLKLAGDVESNP | |
| | GPPAELEVECATQLRRFGDKLNFRQKLLGSGVKQ | |
| | TLNFDLLKLAGDVESNPGPNLWAAQRYGRELRR | |
| | MSDEFVDSFKKGGSGVKQTLNFDLLKLAGDVESN | |
| | PGPPAELEVECATQLRRFGDKLNFRQKLLGSGVK | |
| | QTLNFDLLKLAGDVESNPGPNLWAAQRYGRELR | |
| | RMSDEFVDSFKKGGSGVKQTLNFDLLKLAGDVES | |
| | NPGPPAELEVECATQLRRFGDKLNFRQKLLGSGV | |
| | KQTLNFDLLKLAGDVESNPGPNLWAAQRYGREL | |
| | RRMSDEFVDSFKKGGSGVKQTLNFDLLKLAGDVE | |
| | SNPGPPAELEVECATQLRRFGDKLNFRQKLLGSG | |
| | VKQTLNFDLLKLAGDVESNPGPNLWAAQRYGRE | |
| | LRRMSDEFVDSFKKGGSGVKQTLNFDLLKLAGDV | |
| | ESNPGPGKPIPNPLLGLDST | |
| (Noxa-F2A-BadBH3- | MPAELEVECATQLRRFGDKLNFRQKLLGSGVKQ | 172 |
| F2A)\hat{ }10_cV5 | TLNFDLLKLAGDVESNPGPNLWAAQRYGRELRR | |
| | MSDEFVDSFKKGGSGVKQTLNFDLLKLAGDVES | |
| | NPGPPAELEVECATQLRRFGDKLNFRQKLLGSG | |
| | VKQTLNFDLLKLAGDVESNPGPNLWAAQRYGR | |
| | ELRRMSDEFVDSFKKGGSGVKQTLNFDLLKLAG | |
| | DVESNPGPPAELEVECATQLRRFGDKLNFRQKLL | |
| | GSGVKQTLNFDLLKLAGDVESNPGPNLWAAQR | |
| | YGRELRRMSDEFVDSFKKGGSGVKQTLNFDLLKL | |
| | AGDVESNPGPPAELEVECATQLRRFGDKLNFRQ | |
| | KLLGSGVKQTLNFDLLKLAGDVESNPGPNLWAA | |
| | QRYGRELRRMSDEFVDSFKKGGSGVKQTLNFDL | |
| | LKLAGDVESNPGPPAELEVECATQLRRFGDKLNF | |
| | RQKLLGSGVKQTLNFDLLKLAGDVESNPGPNLW | |
| | AAQRYGRELRRMSDEFVDSFKKGGSGVKQTLNF | |
| | DLLKLAGDVESNPGPPAELEVECATQLRRFGDKL | |
| | NFRQKLLGSGVKQTLNFDLLKLAGDVESNPGPNL | |
| | WAAQRYGRELRRMSDEFVDSFKKGGSGVKQTLN | |
| | FDLLKLAGDVESNPGPPAELEVECATQLRRFGDKL | |
| | NFRQKLLGSGVKQTLNFDLLKLAGDVESNPGPNL | |
| | WAAQRYGRELRRMSDEFVDSFKKGGSGVKQTL | |
| | NFDLLKLAGDVESNPGPPAELEVECATQLRRFGD | |
| | KLNFRQKLLGSGVKQTLNFDLLKLAGDVESNPGP | |
| | NLWAAQRYGRELRRMSDEFVDSFKKGGSGVKQ | |
| | TLNFDLLKLAGDVESNPGPPAELEVECATQLRRF | |
| | GDKLNFRQKLLGSGVKQTLNFDLLKLAGDVESN | |
| | PGPNLWAAQRYGRELRRMSDEFVDSFKKGGSG | |
| | VKQTLNFDLLKLAGDVESNPGPPAELEVECATQL | |
| | RRFGDKLNFRQKLLGSGVKQTLNFDLLKLAGDVE | |
| | SNPGPNLWAAQRYGRELRRMSDEFVDSFKKGG | |
| | SGVKQTLNFDLLKLAGDVESNPGPGKPIPNPLLGL | |
| | DST | |
| (Puma-F2A-BimBH3- | MEEQWAREIGAQLRRMADDLNAQYERRGSGVK | 173 |
| F2A)\hat{ }3_cV5_DX | QTLNFDLLKLAGDVESNPGPDMRPEIWIAQELRR | |
| | IGDEFNAYYARRGSGVKQTLNFDLLKLAGDVESN | |
| | PGPEEQWAREIGAQLRRMADDLNAQYERRGSG | |
| | VKQTLNFDLLKLAGDVESNPGPDMRPEIWIAQEL | |
| | RRIGDEFNAYYARRGSGVKQTLNFDLLKLAGDVE | |
| | SNPGPEEQWAREIGAQLRRMADDLNAQYERRG | |
| | SGVKQTLNFDLLKLAGDVESNPGPDMRPEIWIAQ | |
| | ELRRIGDEFNAYYARRGSGVKQTLNFDLLKLAGDV | |
| | ESNPGPGKPIPNPLLGLDST | |
| (SuperPumaBH3.F2A)\hat{ }3_cV5_ | MQWAREIGAQLRRIGDDLNAQYERRRQGSGVK | 174 |
| DNA2.0 | QTLNFDLLKLAGDVESNPGPQWAREIGAQLRRIG | |
| | DDLNAQYERRRQGSGVKQTLNFDLLKLAGDVES | |
| | NPGPQWAREIGAQLRRIGDDLNAQYERRRQGSG | |
| | VKQTLNFDLLKLAGDVESNPGPGSGVKQTLNFDL | |
| | LKLAGDVESNPGPGKPIPNPLLGLDST | |
| (Puma-F2A-BimBH3- | MEEQWAREIGAQLRRMADDLNAQYERRGSGVK | 175 |
| F2A)\hat{ }3_cV5_DX_DNA2.0 | QTLNFDLLKLAGDVESNPGPDMRPEIWIAQELRR | |
| | IGDEFNAYYARRGSGVKQTLNFDLLKLAGDVESN | |
| | PGPEEQWAREIGAQLRRMADDLNAQYERRGSG | |
| | VKQTLNFDLLKLAGDVESNPGPDMRPEIWIAQEL | |
| | RRIGDEFNAYYARRGSGVKQTLNFDLLKLAGDVE | |
| | SNPGPEEQWAREIGAQLRRMADDLNAQYERRG | |
| | SGVKQTLNFDLLKLAGDVESNPGPDMRPEIWIAQ | |
| | ELRRIGDEFNAYYARRGSGVKQTLNFDLLKLAGDV | |
| | ESNPGPGKPIPNPLLGLDST | |
| (SuperPumaBH3.F2A)\hat{ }5_cV5_ | MQWAREIGAQLRRIGDDLNAQYERRRQGSGVK | 176 |
| DNA2.0 | QTLNFDLLKLAGDVESNPGPQWAREIGAQLRRIG | |
| | DDLNAQYERRRQGSGVKQTLNFDLLKLAGDVES | |
| | NPGPQWAREIGAQLRRIGDDLNAQYERRRQGSG | |
| | VKQTLNFDLLKLAGDVESNPGPQWAREIGAQLR | |
| | RIGDDLNAQYERRRQGSGVKQTLNFDLLKLAGDV | |
| | ESNPGPQWAREIGAQLRRIGDDLNAQYERRRQG | |
| | SGVKQTLNFDLLKLAGDVESNPGPGKPIPNPLLGL | |
| | DST | |
| (pumaBH3.F2A)\hat{ }5_cV5_DNA2.0 | MEEQWAREIGAQLRRMADDLNAQYERRGSGVK | 177 |
| | QTLNFDLLKLAGDVESNPGPEEQWAREIGAQLRR | |
| | MADDLNAQYERRGSGVKQTLNFDLLKLAGDVES | |
| | NPGPEEQWAREIGAQLRRMADDLNAQYERRGS | |
| | GVKQTLNFDLLKLAGDVESNPGPEEQWAREIGA | |
| | QLRRMADDLNAQYERRGSGVKQTLNFDLLKLAG | |
| | DVESNPGPEEQWAREIGAQLRRMADDLNAQYE | |
| | RRGSGVKQTLNFDLLKLAGDVESNPGPGKPIPNP | |
| | LLGLDST | |
| (KittyCatBH3.F2A)\hat{ }5_cV5_DNA2. | MQWAREIGAQERREADDENAQYERRRQGSGV | 178 |
| 0 | KQTLNFDLLKLAGDVESNPGPQWAREIGAQERR | |
| | EADDENAQYERRRQGSGVKQTLNFDLLKLAGDV | |
| | ESNPGPQWAREIGAQERREADDENAQYERRRQ | |
| | GSGVKQTLNFDLLKLAGDVESNPGPQWAREIGA | |
| | QERREADDENAQYERRRQGSGVKQTLNFDLLKL | |
| | AGDVESNPGPQWAREIGAQERREADDENAQYE | |
| | RRRQGSGVKQTLNFDLLKLAGDVESNPGPGKPIP | |
| | NPLLGLDST | |
| (KittyCatBH3.F2A)\hat{ }10_cV5_DNA | MQWAREIGAQERREADDENAQYERRRQGSGV | 179 |
| 2.0 | KQTLNFDLLKLAGDVESNPGPQWAREIGAQERR | |
| | EADDENAQYERRRQGSGVKQTLNFDLLKLAGDV | |
| | ESNPGPQWAREIGAQERREADDENAQYERRRQ | |
| | GSGVKQTLNFDLLKLAGDVESNPGPQWAREIGA | |
| | QERREADDENAQYERRRQGSGVKQTLNFDLLKL | |
| | AGDVESNPGPQWAREIGAQERREADDENAQYE | |
| | RRRQGSGVKQTLNFDLLKLAGDVESNPGPQWA | |
| | REIGAQERREADDENAQYERRRQGSGVKQTLNF | |
| | DLLKLAGDVESNPGPQWAREIGAQERREADDE | |
| | NAQYERRRQGSGVKQTLNFDLLKLAGDVESNPG | |
| | PQWAREIGAQERREADDENAQYERRRQGSGVK | |
| | QTLNFDLLKLAGDVESNPGPQWAREIGAQERRE | |
| | ADDENAQYERRRQGSGVKQTLNFDLLKLAGDVE | |
| | SNPGPQWAREIGAQERREADDENAQYERRRQG | |
| | SGVKQTLNFDLLKLAGDVESNPGPQWAREIGAQ | |
| | ERREADDENAQYERRRQGSGVKQTLNFDLLKLA | |
| | GDVESNPGPGKPIPNPLLGLDST | |
| (pumaBH3.F2A)\hat{ }10_cV5_DNA2. | MEEQWAREIGAQLRRMADDLNAQYERRGSGVK | 180 |
| 0 | QTLNFDLLKLAGDVESNPGPEEQWAREIGAQLRR | |
| | MADDLNAQYERRGSGVKQTLNFDLLKLAGDVES | |
| | NPGPEEQWAREIGAQLRRMADDLNAQYERRGS | |
| | GVKQTLNFDLLKLAGDVESNPGPEEQWAREIGA | |
| | QLRRMADDLNAQYERRGSGVKQTLNFDLLKLAG | |
| | DVESNPGPEEQWAREIGAQLRRMADDLNAQYE | |
| | RRGSGVKQTLNFDLLKLAGDVESNPGPEEQWAR | |
| | EIGAQLRRMADDLNAQYERRGSGVKQTLNFDLL | |
| | KLAGDVESNPGPEEQWAREIGAQLRRMADDLN | |
| | AQYERRGSGVKQTLNFDLLKLAGDVESNPGPEEQ | |
| | WAREIGAQLRRMADDLNAQYERRGSGVKQTLN | |
| | FDLLKLAGDVESNPGPEEQWAREIGAQLRRMAD | |
| | DLNAQYERRGSGVKQTLNFDLLKLAGDVESNPGP | |
| | EEQWAREIGAQLRRMADDLNAQYERRGSGVKQ | |
| | TLNFDLLKLAGDVESNPGPGKPIPNPLLGLDST | |
| (Puma-F2A-BimBH3- | MEEQWAREIGAQLRRMADDLNAQYERRGSGVK | 181 |
| F2A)\hat{ }5_cV5_DNA2.0 | QTLNFDLLKLAGDVESNPGPDMRPEIWIAQELRR | |
| | IGDEFNAYYARRGSGVKQTLNFDLLKLAGDVESN | |
| | PGPEEQWAREIGAQLRRMADDLNAQYERRGSG | |
| | VKQTLNFDLLKLAGDVESNPGPDMRPEIWIAQEL | |
| | RRIGDEFNAYYARRGSGVKQTLNFDLLKLAGDVE | |
| | SNPGPEEQWAREIGAQLRRMADDLNAQYERRG | |
| | SGVKQTLNFDLLKLAGDVESNPGPDMRPEIWIAQ | |
| | ELRRIGDEFNAYYARRGSGVKQTLNFDLLKLAGDV | |
| | ESNPGPEEQWAREIGAQLRRMADDLNAQYERR | |
| | GSGVKQTLNFDLLKLAGDVESNPGPDMRPEIWIA | |
| | QELRRIGDEFNAYYARRGSGVKQTLNFDLLKLAG | |
| | DVESNPGPEEQWAREIGAQLRRMADDLNAQYE | |
| | RRGSGVKQTLNFDLLKLAGDVESNPGPDMRPEI | |
| | WIAQELRRIGDEFNAYYARRGSGVKQTLNFDLLKL | |
| | AGDVESNPGPGKPIPNPLLGLDST | |
| (SuperPumaBH3.F2A)\hat{ }10_cV5_ | MQWAREIGAQLRRIGDDLNAQYERRRQGSGV | 182 |
| DNA2.0 | KQTLNFDLLKLAGDVESNPGPQWAREIGAQLR | |
| | RIGDDLNAQYERRRQGSGVKQTLNFDLLKLAG | |
| | DVESNPGPQWAREIGAQLRRIGDDLNAQYERR | |
| | RQGSGVKQTLNFDLLKLAGDVESNPGPQWAR | |
| | EIGAQLRRIGDDLNAQYERRRQGSGVKQTLNF | |
| | DLLKLAGDVESNPGPQWAREIGAQLRRIGDDL | |
| | NAQYERRRQGSGVKQTLNFDLLKLAGDVESNP | |
| | GPQWAREIGAQLRRIGDDLNAQYERRRQGSG | |
| | VKQTLNFDLLKLAGDVESNPGPQWAREIGAQL | |
| | RRIGDDLNAQYERRRQGSGVKQTLNFDLLKLA | |
| | GDVESNPGPQWAREIGAQLRRIGDDLNAQYE | |
| | RRRQGSGVKQTLNFDLLKLAGDVESNPGPOW | |
| | AREIGAQLRRIGDDLNAQYERRRQGSGVKQTL | |
| | NFDLLKLAGDVESNPGPQWAREIGAQLRRIGD | |
| | DLNAQYERRRQGSGVKQTLNFDLLKLAGDVES | |
| | NPGPGKPIPNPLLGLDST | |
| (KittyCatBH3.F2A)\hat{ }3_cV5 | MQWAREIGAQERREADDENAQYERRRQGSGVK | 183 |
| | QTLNFDLLKLAGDVESNPGPQWAREIGAQERRE | |
| | ADDENAQYERRRQGSGVKQTLNFDLLKLAGDVE | |
| | SNPGPQWAREIGAQERREADDENAQYERRRQG | |
| | SGVKQTLNFDLLKLAGDVESNPGPGKPIPNPLLGL | |
| | DST | |
| (KittyCatBH3.P2A)\hat{ }3_cV5 | MQWAREIGAQERREADDENAQYERRRQGSGAT | 184 |
| | NFSLLKQAGDVEENPGPQWAREIGAQERREADD | |
| | ENAQYERRRQGSGATNFSLLKQAGDVEENPGPQ | |
| | WAREIGAQERREADDENAQYERRRQGSGVKQTL | |
| | NFDLLKLAGDVESNPGPGKPIPNPLLGLDST | |
| PumaBH3(x3.P2A).v5_miR122 | MEEQWAREIGAQLRRMADDLNAQYERRGSGAT | 185 |
| | NFSLLKQAGDVEENPGPEEQWAREIGAQLRRMA | |
| | DDLNAQYERRGSGATNFSLLKQAGDVEENPGPE | |
| | EQWAREIGAQLRRMADDLNAQYERRGKPIPNPL | |
| | LGLDST | |
| (PUMA BH3.GGGSx3)x3.V5 | MEEQWAREIGAQLRRMADDLNAQYERRGGGS | 186 |
| | GGGSGGGSEEQWAREIGAQLRRMADDLNAQYE | |
| | RRGGGSGGGSGGGSEEQWAREIGAQLRRMADD | |
| | LNAQYERRGKPIPNPLLGLDST | |
| (PUMA BH3.GGGSx3)x3.V5_DX | MEEQWAREIGAQLRRMADDLNAQYERRGGGS | 187 |
| | GGGSGGGSEEQWAREIGAQLRRMADDLNAQYE | |
| | RRGGGSGGGSGGGSEEQWAREIGAQLRRMADD | |
| | LNAQYERRGKPIPNPLLGLDST | |
| PUMA BH3.GGGSx3.PUMA | MEEQWAREIGAQLRRMADDLNAQYERRGGGS | 188 |
| BH3.V5 | GGGSGGGSEEQWAREIGAQLRRMADDLNAQYE | |
| | RRGKPIPNPLLGLDST | |
| PumaBH3(x3.P2A).v5_miR122 | MEEQWAREIGAQLRRMADDLNAQYERRGSGAT | 189 |
| | NFSLLKQAGDVEENPGPEEQWAREIGAQLRRMA | |
| | DDLNAQYERRGSGATNFSLLKQAGDVEENPGPE | |
| | EQWAREIGAQLRRMADDLNAQYERRGKPIPNPL | |
| | LGLDST | |

The modified mRNA constructs contained a 5′ UTR having the sequence shown in SEQ ID NO: 72, a 3′ UTR having the sequence shown in SEQ ID NO: 73, contained a Cap 1 5′ Cap (7mG(5′)ppp(5′)NlmpNp), a poly A tail of 100 nucleotides and were fully modified with 1-methyl-pseudouridine (m1ψ). The open reading frame (ORF) amino acid sequence shown in Table 15 encoded by the mRNA constructs include an epitope tag sequence incorporated into the sequence. Amino acid sequences corresponding to the constructs of SEQ ID NOs: 142-189 but without the epitope tag are shown in SEQ ID NOs: 190-237. Nucleotide sequences encoding the amino acid sequences shown in SEQ ID NOs: 142-189 are shown in SEQ ID NOs: 238-285.

Expression of the polypeptides from the mRNA constructs was examined using cell free translation methods with wheat germ lysate, as follows. Wheat Germ Lysate (WGL; Promega, Cat. #L4380) was prepared according to the manufacturer's instructions by adding 28 ul of potassium acetate to 200 ul of WGL. mRNA constructs were diluted to 250 ng/ul and heat denatured at 65° C. for 3 minutes. Individual translation reactions were prepared containing 12.5 ul of prepared WGL, 2 ul methionine-free amino acid mix, 2 ul of 5 mM methionine, 2 ul of mRNA at 250 ng/ul (500 ng per reaction) and 6.5 ul sterile water for a final volume of 25 ul. Reactions were incubated at 25° C. for 120 minutes. After incubation, a portion of the reaction mixture (e.g., 8%, 20%) was mixed with LDS/DTT and loaded onto a 12% bis/tris gel. Following electrophoresis, the gel was blotted onto a 0.2 um nitrocellulose membrane using the iBlot transfer system. The membrane was blocked with 5% non-fat dry milk in PBS. The blocked membrane was then incubated with a mouse anti-V5 antibody (Life Tech 1:8K in PBS-T), which binds to the epitope tag encoded by the mRNA constructs, for 1 hour at room temperature, followed by a labeled rat anti-mouse antibody (BD, 1:100K in PBS-T) for 1 hour at room temperature.

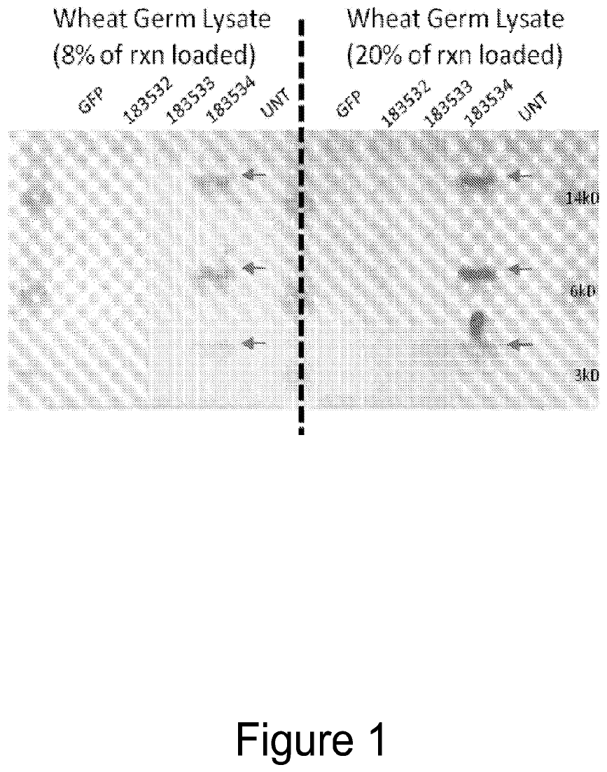

Representative results of the cell free translation experiment are shown in FIG. 1, which demonstrate that expression of an mmRNA construct encoding 3 copies of the PUMA BH3 domain, with an F2A cleavage site in between each BH3 domain (referred to as “PumaBH3(x3.F2A).v5_Hs3UPCRfree” in Table 15 and as 183534 in FIG. 1), using the WGL in a cell free translation system led to detection of mono-, di- and trimeric BH3 domain species.

### Example 2

### Rapid Induction of Apoptosis and Cell Killing by Transfection of Hep3B Cells with mRNA Encoding Multimeric BH3 Domains

To determine whether multimeric BH3 domain constructs could induce apoptosis, Hep3B hepatocellular carcinoma cells were transfected with varying doses of a series of different mmRNA constructs encoding either 3 or 5 copies of the PUMA BH3 domain, with intervening F2A linkers between each BH3 domain. Certain multimeric BH3 domain constructs also contained a miR122 binding site or a miR142.3p binding site or both of these miR binding sites. As a positive control, an SQT-PUMA-BH3 mmRNA construct encoding a fusion polypeptide of the SQT scaffold linked to a single PUMA BH3 domain, which was known to induce apoptosis, was used. As a negative control, an “empty” SQT mmRNA construct, encoding only the SQT scaffold with no BH3 domain, was used.

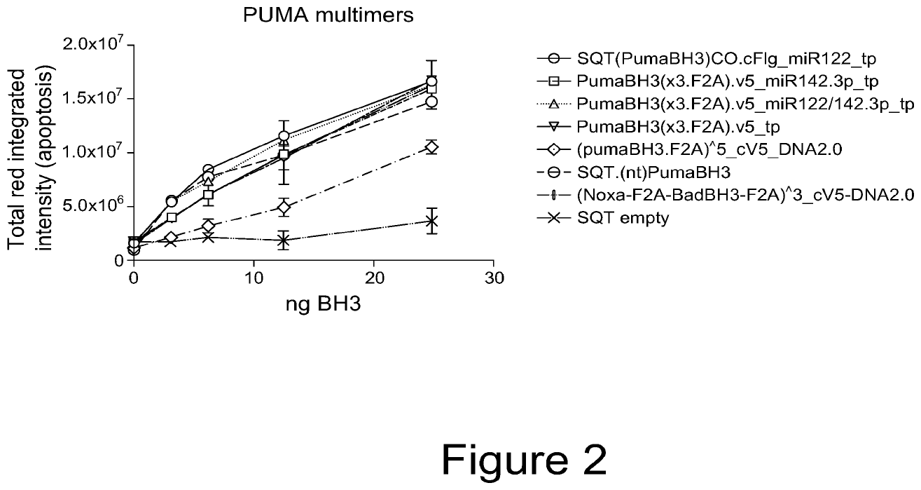

20,000 Hep3B cells/well were plated in 96 well plates and the cells were transfected with varying doses of the mmRNA constructs using Lipofectamine 2000 (Life Technologies) in the presence of YOYO-3 (Life Technologies), a DNA dye that is taken up preferentially by dead cells that is used to measure the extent of cell death. The results for apoptosis at 24 hours post-transfection are shown in the graph of FIG. 2. The results demonstrate that all mmRNA constructs encoding multimeric BH3 domains induced apoptosis of Hep3B cells, with the PUMA BH3×3 multimeric constructs (containing intervening F2A cleavage sites and either with or without miR binding sites) exhibiting similar potency to the SQT-PUMA-BH3 construct and a higher potency than the PUMA BH3×5 multimeric construct.

### Example 3

### Lipid Nanoparticle-Formulated Multimeric BH3 Constructs Induce Apoptosis in Hep3B Cells

The efficacy of mmRNA constructs encoding multimeric BH3 domains in inducing cell death was tested in the Hep3b hepatocellular carcinoma cell lines using lipid nanoparticle (LNP)-formulated constructs. Hep3B cells were treated with varying doses of MC3-formulated LNPs containing varying doses of a series of different mmRNA constructs encoding either 3 or 5 copies of the PUMA BH3 domain, with intervening F2A linkers between each BH3 domain. As a positive control, LNPs containing an SQT-PUMA-BH3 mmRNA construct, encoding a fusion polypeptide of the SQT scaffold linked to a single PUMA BH3 domain, which was known to induce apoptosis, was used. As a negative control, LNPs containing an “empty” SQT mmRNA construct, encoding only the SQT scaffold with no BH3 domain, was used.

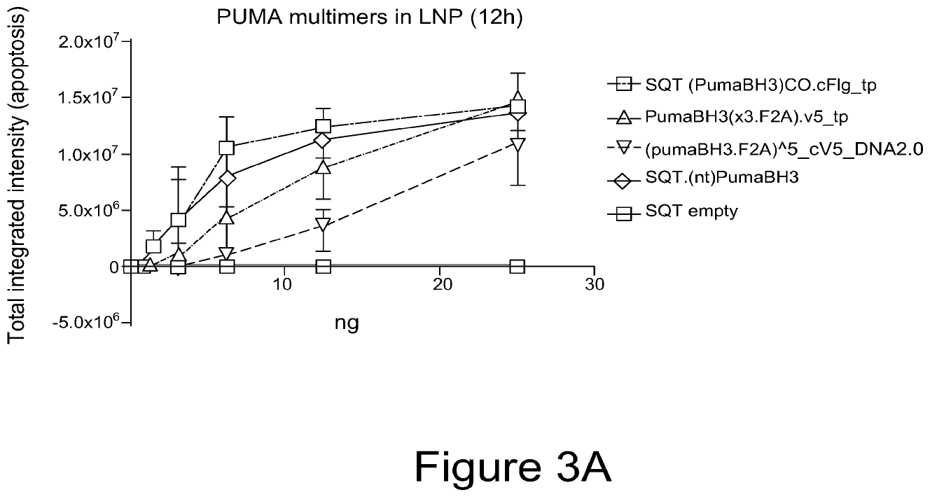

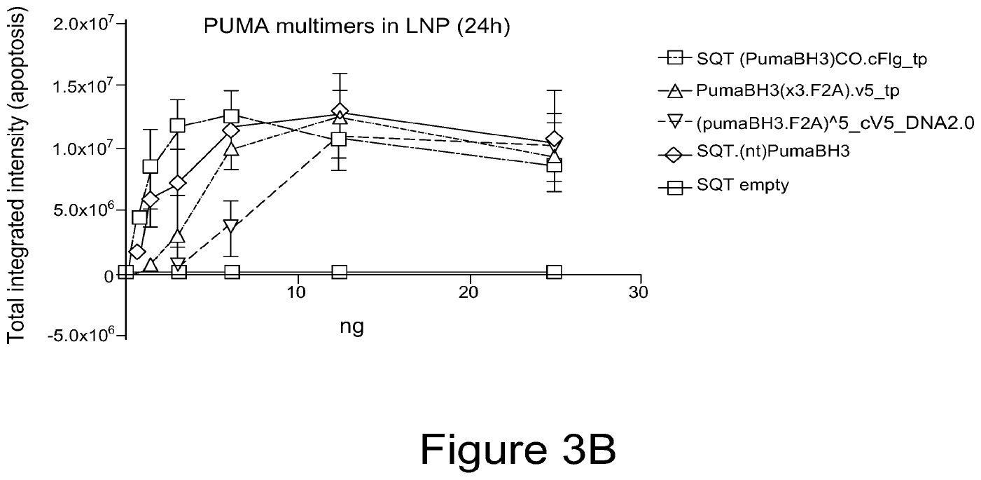

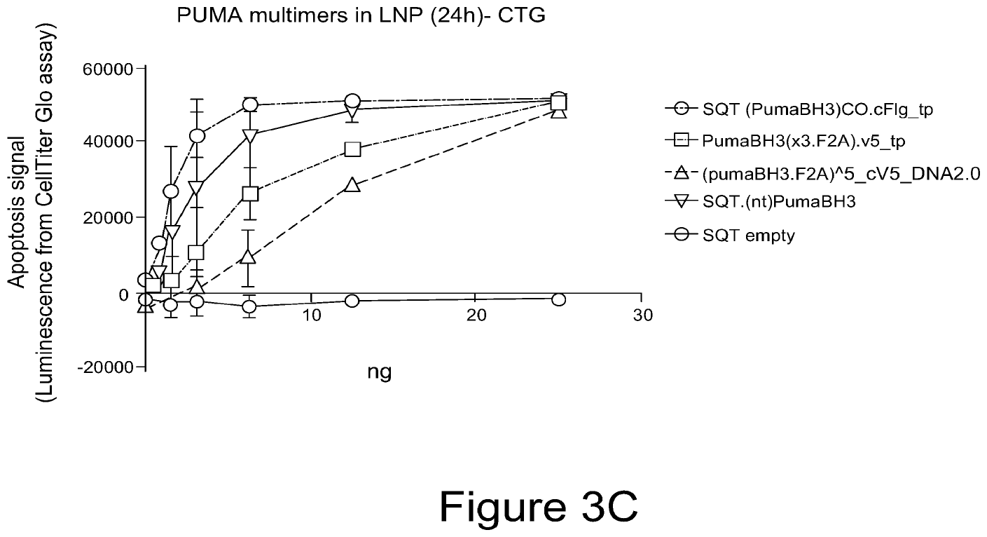

20,000 Hep3B cells/well were plated in 96 well plates and the cells were treated with varying doses of the LNPs containing the mmRNA constructs. Cell death was measured by Essen Caspase 3/7 reagent staining (total integrated intensity apoptosis) after 12 or 24 hours. The results of these experiments are shown in FIGS. 3A (12 hours) and 3B (24 hours). Cell death also was measured using CellTiterGlo (CTG) assay at 24 hours (FIG. 3C). The results demonstrate that LNPs containing the mmRNA constructs encoding multimeric BH3 domains induced apoptosis of Hep3B cells, with the PUMA BH3×3 multimeric construct exhibiting similar potency to the SQT-PUMA-BH3 construct and a higher potency than the PUMA BH3×5 multimeric construct.

### Example 4

### Rapid Induction of Apoptosis and Cell Killing by Transfection of Hep3B Cells with mRNA Encoding Truncated Bid Containing a BH3 Domain

To determine whether truncated BID constructs containing a BH3 domain could induce apoptosis, Hep3B hepatocellular carcinoma cells were transfected with varying doses of truncated BID (tBID) BH3 domain-containing mmRNA constructs. One construct encoded a single copy of amino acids 61-195 of the BID protein that includes its BH3 domain (with a FLAG epitope tag). The open reading frame (ORF) amino acid sequence and corresponding nucleotide sequence encoding the ORF a.a. sequence are shown in SEQ ID NOs: 289 and 290, respectively. The sequence for the corresponding construct without the FLAG epitope tag is shown in SEQ ID NO: 291. A second construct encoded a single copy of amino acids 77-195 of the BID protein that includes its BH3 domain (with a FLAG epitope tag). The open reading frame (ORF) amino acid sequence and corresponding nucleotide sequence encoding the ORF a.a. sequence are shown in SEQ ID NOs: 292 and 293, respectively. The sequence for the corresponding construct without the FLAG epitope tag is shown in SEQ ID NO: 294. The open reading frame (ORF) amino acid sequence and corresponding nucleotide sequence encoding the ORF a.a. sequence of a third construct, containing amino acids 61-104 of the BID protein, are shown in SEQ ID NOs: 286 and 287, respectively. The sequence for the corresponding construct without the FLAG epitope tag is shown in SEQ ID NO: 288.

As a positive control, an SQT-PUMA-BH3 mmRNA construct encoding a fusion polypeptide of the SQT scaffold linked to a single PUMA BH3 domain, which was known to induce apoptosis, was used. As a negative control, an “empty” SQT mmRNA construct, encoding only the SQT scaffold with no BH3 domain, was used.

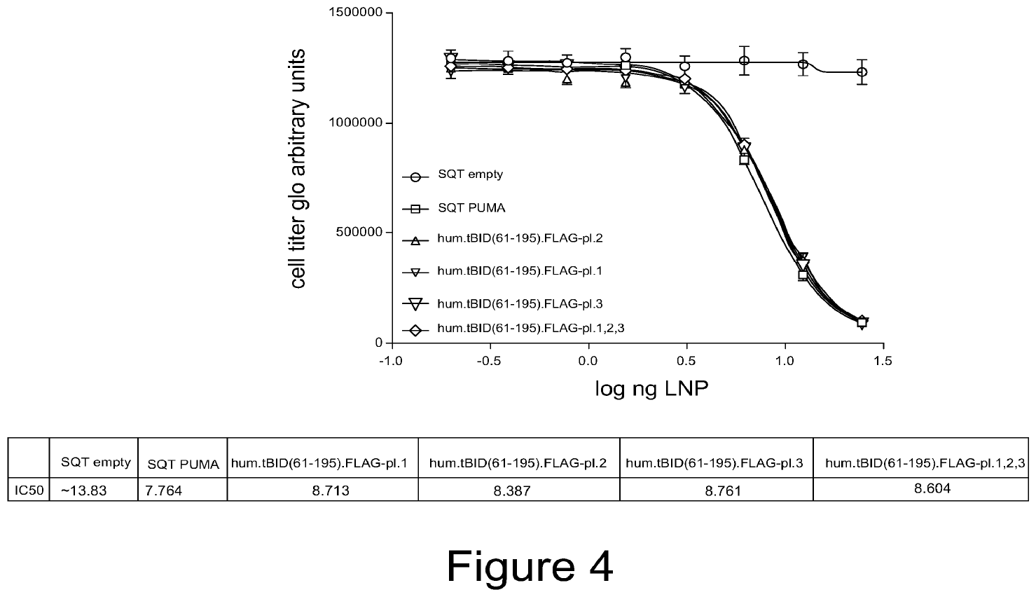

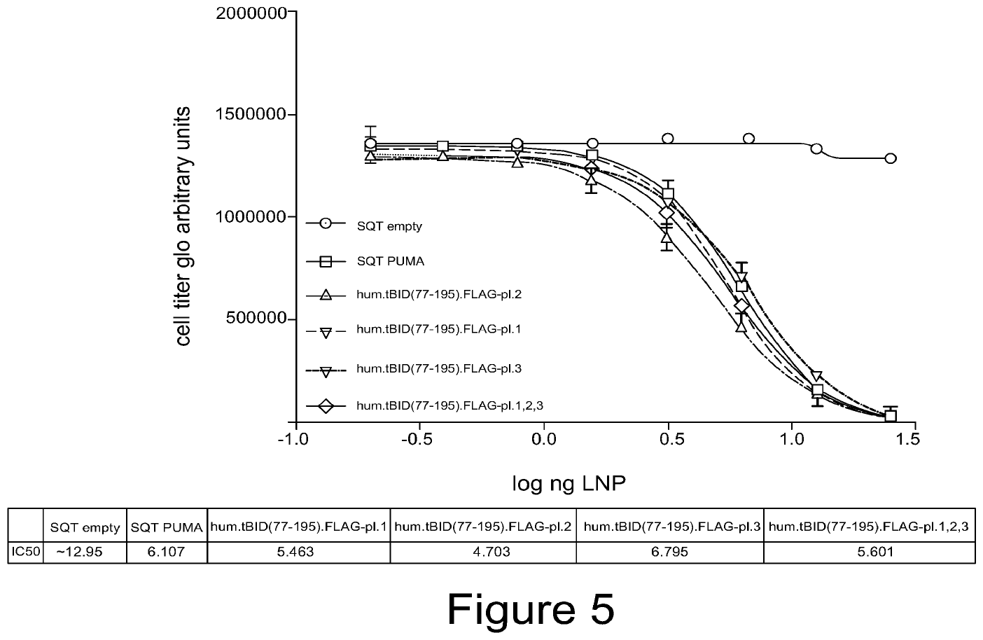

Hep3B cells/well were plated in 96 well plates and the cells were transfected with varying doses of the mmRNA constructs. Cell death was measured using the CellTiterGlo (CTG) assay. The results for the tBID (61-195) constructs are shown in the graph of FIG. 4 (“pl. 1, 2 and 3” refers to results from different plates). The results for the tBID (77-195) constructs are shown in the graph of FIG. 5 (“pl. 1, 2 and 3” refers to results from different plates). The results demonstrate that both of the truncated BID constructs containing its BH3 domain induced apoptosis of Hep3B cells, exhibiting a similar potency to the positive control SQT-PUMA-BH3 construct.

### Example 5

### Synergistic Pro-Apoptotic Effect of Targeting MCL1 in Combination with SQT-BH3 Constructs

In this example, the effect on apoptosis of combining SQT-BH3 mRNA constructs with mRNA constructs specifically targeting MCL1 was examined further. It previously had been observed that combining SQT-BH3 with sorafenib led to synergistic pro-apoptotic effects. Sorafenib can cause downregulation of MCL1 but also affects other pathways and thus unwanted side effects could occur through the use of sorafenib to target MCL1. Furthermore, sorafenib does not cause downregulation of MCL1 in all cell types (e.g., primary hepatocytes). Accordingly, specific anti-MCL1 inhibitors were desired. While not intending to be limited by mechanism, it is thought that by neutralizing MCL1 in tumor cells, the tumor then reverts to sole reliance on BCLXL, BCL2 and/or other prosurvival members of the family as the prosurvival mechanism/pathway. Accordingly, use of SQT-BH3 (or any mRNA construct encoding one or more BH3 domains), which specifically destroys BCLXL, BCL2 and/or prosurvival members of the family then leads to better tumor killing when used in combination with an anti-MCL1 agent.

A first series of experiments was conducted to determine whether specific targeting of MCL1 recapitulates sorafenib synergy with SQT-BH3 in cells in vitro. In these experiments, MCL1 was targeted using a commercially available siRNA (Dharmacon, ON-TARGETplus Human MCL1 (4170) siRNA-SMARTpool, #L-004501-00-0005). Western blotting experiments confirmed that treatment of Hep3B cells with this siRNA or with 10 uM sorafenib for 24 hours downregulated MCL1, whereas treatment with a non-targeting control siRNA or with PBS did not downregulate MCL1.

Next, Hep3B cells and primary hepatocytes were treated with either SQT-PUMA-BH3 or SQT-Bad-BH3 in combination with either siMCL1 or sorafenib. Controls were an empty SQT construct (“SQT-dummy”), a non-targeting siRNA and PBS. For treatment of Hep3B cells, 20,000 Hep3B cells/well per plated in 96-well plates. At Day 1, some wells were transfected with siMCL1 (Dharmacon, ON-TARGETplus Human MCL1 (4170) siRNA-SMARTpool, #L-004501-00-0005) or non-targeting siRNA (ON-TARGETplus Non-targeting Control Pool, #D-001810-10-05). At Day 3, cells were treated with the 10 uM sorafenib tosylate (Selleck Chemicals) or PBS, and transfected with SQT-dummy or SQT-PUMA or SQT-Bad. 24 hours after treatment, YOYO®-3 iodide (Life Technologies) viability dye was added to each well and cell death was measured on an IncuCyte ZOOM® using YOYO-3 fluorescence as a readout (=“Apoptosis”). For treatment of primary hepatocytes, 18,000 mouse hepatocytes (Gibco) were seeded in each well of a 96-well plate. At Day 1, some wells were transfected with siMCL1 (Dharmacon, SMARTpool: ON-TARGETplus Mcl siRNA, #L-062229-00-0005) or non-targeting siRNA (ON-TARGETplus Non-targeting Control Pool, #D-001810-10-05). At Day 3, cells were treated with the 10 uM sorafenib tosylate (Selleck Chemicals) or PBS, and transfected with SQT-dummy or SQT-PUMA or SQT-Bad. 24 hours after treatment, Caspase 3/7 reagent (EssenBio) apoptosis dye was added to each well and cell death was measured on an IncuCyte ZOOM® using the caspase 3/7 reagent fluorescence as a readout (=“Apoptosis”).

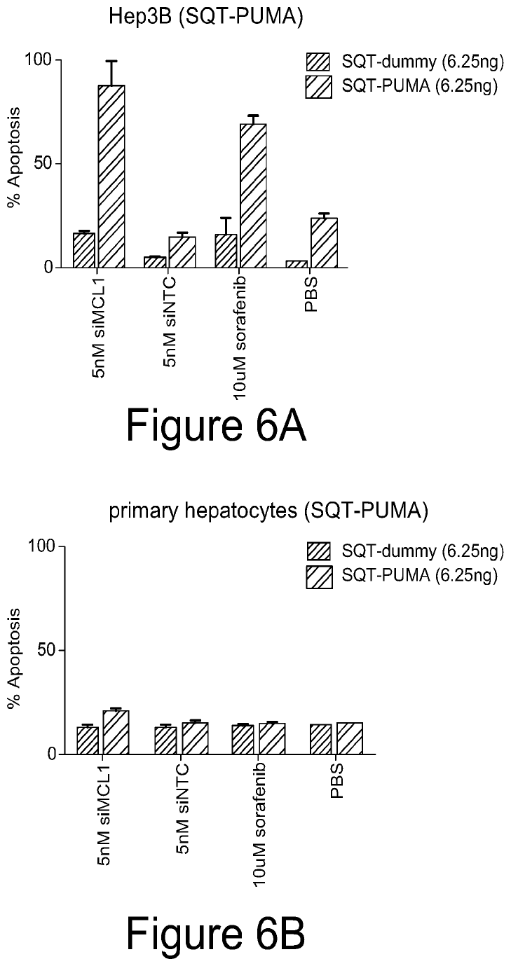

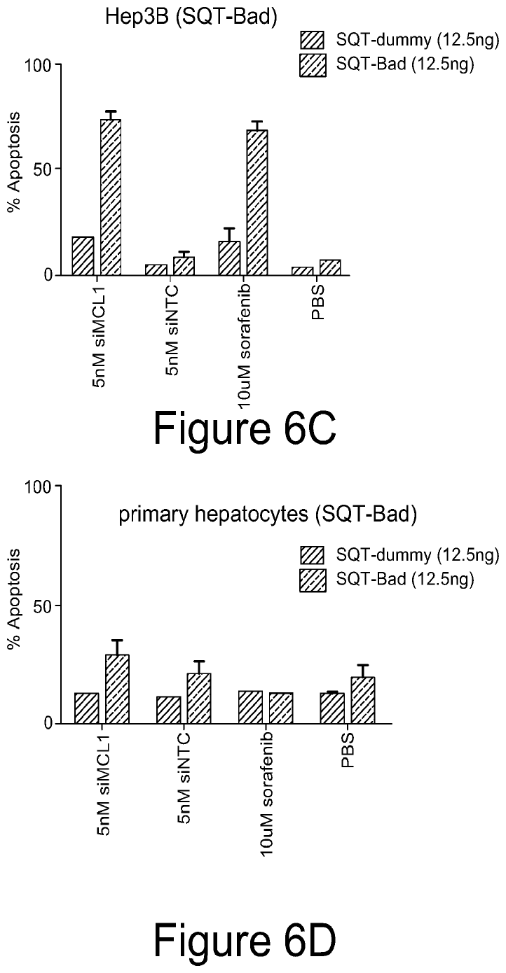

The results are shown in FIGS. 6A-D, wherein FIGS. 6A and 6C show the results for the Hep3B cells and FIGS. 6B and 6D show the results for the primary hepatocytes. FIGS. 6A and 6B show the results for treatment with SQT-PUMA-BH3. FIGS. 6C and 6D show the results for treatment with SQT-Bad-BH3. The results demonstrate that in primary hepatocytes, treatment with either SQT-PUMA-BH3 or SQT-Bad-BH3, in combination with either siMCL1, sorafenib or controls, did not lead to increased apoptosis as compared to treatment with the control SQT-dummy construct. In contrast, treatment of the Hep3B cells with either SQT-PUMA-BH3 or SQT-Bad-BH3, in combination with either siMCL1 or sorafenib, led to a marked increase in apoptosis as compared to treatment with the SQT-BH3 construct in combination with non-targeting siRNA or PBS. These results demonstrate that in the hepatocellular carcinoma cells (but not primary hepatocytes) specific targeting of MCL1 recapitulates the pro-apoptotic synergy previously observed with SQT-BH3 in combination with sorafenib.

Therefore, a series of anti-MCL1 mmRNA constructs were designed, the amino acid sequences of the open reading frames of which are shown below in Table 16:

TABLE 16

| | ORF Amino Acid | Nucleotide |
|---|---|---|
| Name | SEQ ID NO: | SEQ ID NO: |
| SQTanti-Mcl1.(nt)BimBH3.2A.v5 | 107 | 127 |
| BimBH3.2A(x3.F2A).v5_miR122 | 108 | 128 |
| SQT.(nt)Mcl1.BH3.cv5 | 109 | 129 |
| Mcl1.BH3(x3.F2A).cV5 | 110 | 130 |
| antiMcl1.MS1(x3.F2A).cV5 | 111 | 131 |
| antiMcl1.MS2(x3.F2A).cV5 | 112 | 132 |
| antiMcl1.MS3(x3.F2A).cV5 | 113 | 133 |
| antiMcl1.SB-02(x3.F2A).cV5 | 114 | 134 |
| antiMcl1.SB-03(x3.F2A).cV5 | 115 | 135 |
| antiMcl1.SB-04(x3.F2A).cV5 | 116 | 136 |

The modified mRNA constructs contained a 5′ UTR having the sequence shown in SEQ ID NO: 72, a 3′ UTR having the sequence shown in SEQ ID NO: 73, a Cap 1 5′ Cap (7mG(5′)ppp(5′)NmpNp), a poly A tail of 100 nucleotides and were fully modified with 1-methyl-pseudouridine (m1ψ). The open reading frame (ORF) amino acid sequence shown in Table 16 encoded by the mRNA constructs include an epitope tag sequence (v5) incorporated into the sequence. Sequences corresponding to SEQ ID NOs: 107-116 but without the epitope tag are shown in SEQ ID NOs: 117-126.

Anti-MCL1 mmRNA constructs were tested for synergistic killing when combined with SQT-PUMA-BH3 in Hep3B cells. 20,000 Hep3B cells were seeded per well in 96-well plates. Each well was transfected with (a) 1.6 ng SQT-empty (“SQT-dummy”) or SQT-PUMA-BH3; and (b) 50 ng or 12.5 ng anti-MCL1 mRNA. Transfections of mRNA were done with Lipofectamine 2000 (L2K). After 48 hours, apoptosis measurements performed using YOYO-3 viability dye in an Incucyte instrument.

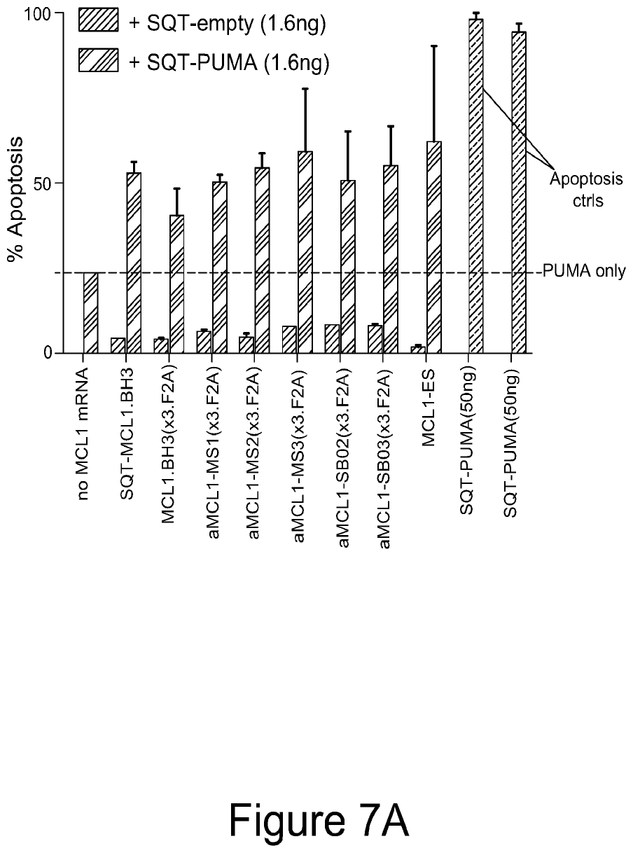

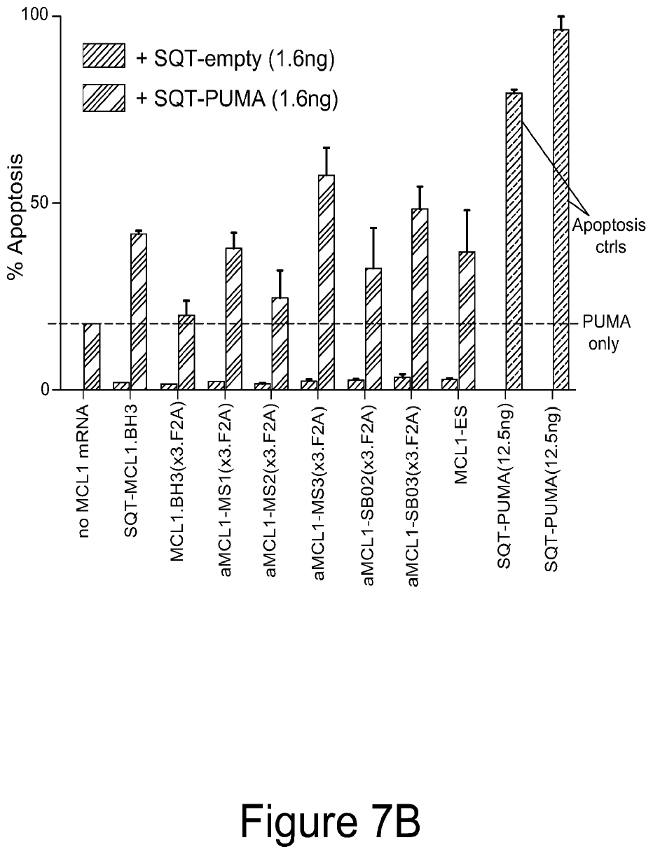

The results are shown in FIGS. 7A-B. FIG. 7A shows the results for treatment with 50 ng anti-MCL1 mRNA; FIG. 7B shows the results for treatment with 12.5 ng anti-MCL1 mRNA. The results demonstrate that at the 50 ng dosage, all anti-MCL1 constructs tested demonstrated a synergistic anti-apoptotic effect in combination with SQT-PUMA-BH3, as compared to treatment with SQT-PUMA-BH3 alone.

### Example 6

### Evaluation of PEG-Lipid Ratios in Lipid Nanoparticles

Optimal molar percentages of PEG-Lipids for in vivo targeted delivery of lipid nanoparticles (LNPs) to tumors were evaluated by varying the molar percentages of different PEG-Lipids in LNPs and testing their physicochemical properties in the CT-26 and Hep3B mouse tumor models.

### Preparation and Characterization of PEG-Lipid LNPs

Lipid nanoparticle formulations were prepared that were composed of: (i) 50 mol % DLin-KC2-DMA (cationic lipid or ionizable amino lipid); (ii) 10 mol % DODMA-DSPC (non-cationic lipid); (iii) 30-39.5 mol % Cholesterol; and (iv) 0.5-10 mol % PEG-Lipid. The total percentage of Cholesterol+PEG-Lipid=40 mol %. Thus, when the PEG-Lipid was present at 0.5 mol %, the Cholesterol was present at 39.5 mol %, when the PEG-Lipid was present at 1.5 mol %, the Cholesterol was present at 38.5 mol %, when the PEG-Lipid was present at 5 mol %, the Cholesterol was present at 35 mol % and when the PEG-Lipid was present at 10 mol %, the Cholesterol was present at 30 mol %.

Three forms of PEG-Lipid were tested: PEG-DMG, PEG-DSG and PEG-DSPE. PEG-DMG and PEG-DSG have the following structure:

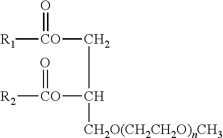

wherein for DMG: R1, R2=C14 and for DSG: R1, R2=C18.
PEG-DSPE has the following structure:

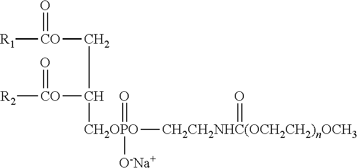

wherein for DSPE: R1, R2=C18.

The PEG-Lipids having longer R1, R2 lipid chains (e.g., DSG and DSPE) are less diffusible than PEG-Lipids having shorter R1, R2 lipid chains (e.g., DMG).

The physiochemical properties of the LNPs containing different percentages of the three different PEG-Lipids was examined, the results of which are shown below in Tables 17-20 below.

TABLE 17

| | Z-Average (diameter in nm) | | | |
|---|---|---|---|---|
| | 0.5 mol % | 1.5 mol % | 5 mol % | 10 mol % |
| PEG-DMG | 184.3 | 77.03 | 62.2 | 53.49 |
| PEG-DSG | 190.9 | 82.52 | 65.64 | 49.81 |
| PEG-DSPE | 179.9 | 84.58 | 58.74 | 47.4 |

These results demonstrate that the 0.5 mol % PEG-containing LNPs were larger than those with a higher mol % PEG-Lipid concentration.

TABLE 18

| | Polydispersity Index (PDI) | | | |
|---|---|---|---|---|
| | 0.5 mol % | 1.5 mol % | 5 mol % | 10 mol % |
| PEG-DMG | 0.069 | 0.149 | 0.205 | 0.213 |
| PEG-DSG | 0.065 | 0.145 | 0.172 | 0.196 |
| PEG-DSPE | 0.077 | 0.079 | 0.255 | 0.287 |

TABLE 19

| | Encapsulation Efficiency (%) | | | |
|---|---|---|---|---|
| | 0.5 mol % | 1.5 mol % | 5 mol % | 10 mol % |
| PEG-DMG | 92 | 97 | 92 | 48 |
| PEG-DSG | 91 | 98 | 97 | 39 |
| PEG-DSPE | 92 | 97 | 97 | 58 |

These results demonstrate there was a lower encapsulation efficiency for LNPs containing 10 mol % PEG-Lipid than for LNPs containing a lower mol % PEG-Lipid concentration.

TABLE 20

| | Zeta Potential (mV) | | | |
|---|---|---|---|---|
| | 0.5 mol % | 1.5 mol % | 5 mol % | 10 mol % |
| PEG-DMG | 0.422 | 2.72 | −4.52 | −1.48 |
| PEG-DSG | 0.477 | −7.05 | −5.36 | −2.2 |
| PEG-DSPE | −2.93 | −1.87 | −2.53 | −2.15 |

### Intratumoral Administration of PEG-Lipid LNPs

The LNP formulations containing varying mol % of different PEG-Lipids were used to administer a reporter mRNA construct into CT-26 tumors in mice. The reporter mRNA construct encoded a luciferase reporter protein to allow for evaluation of the biodistribution of the mRNA encapsulated by the LNPs. The mRNA construct included a Cap 1 5′ cap, and a 140 polyA tail and was fully modified with 1-methylpseudouridine and 5-methyl-cytosine.

LNPs formulations as described above, containing either 0.5 mol % or 1.5 mol % PEG-DMG, PEG-DSG or PEG-DSPE were administered intratumorally into BALB/cAnNCrl mice bearing CT-26 tumors (n=4 per group; average tumor size of 290 mm³) at a single dose of 0.05 mg/kg luciferase mRNA. Controls were PBS alone and naked mRNA luciferase in 8.5% sucrose.

Whole body BLI was performed 2 hours post-treatment. Twenty minutes prior to imaging, mice were injected intraperitoneally with a D-luciferin solution at 150 mg/kg. Animals were then anesthetized and images were acquired with an IVIS Lumina II imaging system (Perkin Elmer). Bioluminescence was measured as total flux (photons/second) of the entire mouse.

Animals were sacrificed and tumors and livers were harvested at 4 hours post-treatment for ex vivo luciferase protein quantification. The tumor:liver expression ratio for the luciferase reporter protein was determined for each of the PEG-Lipids tested and different mol % concentrations tested.

The results from both the BLI analysis and the ex vivo protein quantitation showed that, overall, the lower molar percentages of PEG-Lipid improved the tumor:liver ratio post intra-tumor administration and the LNPs containing 0.5 mol % of any of the three PEGs exhibited a higher tumor:liver expression profile at the time points examined compared to those with 1.5 mol %.

### Systemic Administration of PEG-Lipid LNPs

The LNP formulations containing varying mol % of different PEG-Lipids were used to administer a reporter mRNA construct systemically into mice bearing either a CT-26 subcutaneous tumor or a Hep3B subcutaneous tumor. The same luciferase reporter mRNA construct described above for the intra-tumor studies was used for the systemic administration studies.

LNPs formulations as described above, containing either 1.5 mol %, 5 mol % or 10 mol % PEG-DMG, PEG-DSG or PEG-DSPE were administered intravenously into BALB/c nude mice bearing Hep3B SC tumors (tumor size range=254-876 mm³; average=548 mm³) or CT-26 SC tumors (tumor size range=278-664 mm³; average=405 mm³) (n=4 per group). Mice were injected IV with 100 μl of 0.04 mg/ml RNA, which is a dose of approximately 0.2 mg/kg luciferase mRNA. Control was PBS alone.

Whole body BLI was performed 2, 6, 24 and 48 hours post-treatment. Twenty minutes prior to imaging, mice were injected intraperitoneally with a D-luciferin solution at 150 mg/kg. Animals were then anesthetized and images were acquired with an IVIS Lumina II imaging system (Perkin Elmer). The ventral view was imaged at 15 minutes post D-luciferin administration and the tumor side view was imaged at 20 minutes post D-luciferin administration. Bioluminescence was measured as total flux (photons/second) of the entire mouse.

Animals were sacrificed and tumors and livers were harvested at 4 days post-treatment for ex vivo luciferase protein quantification. The tumor:liver expression ratio for the luciferase reporter protein was determined for each of the PEG-Lipids tested and different mol % concentrations tested.

The results from both the BLI analysis and the ex vivo protein quantitation showed that the expression profiles of the systemically administered LNPs was better in the Hep3B tumor than in the CT-26 tumors. The BLI analysis showed that 1.5 mol % PEG-DSPE and 5 mol % DSG exhibited the most promising Hep3B tumor expression profile (i.e., higher relative liver signal) within the first 2-48 hours post-treatment. The ex vivo protein quantification analysis demonstrated that the following LNPs exhibited the highest average luciferase protein levels in tumors 52 hours post-treatment: 1.5 mol % PEG-DSG, 5 mol % PEG-DSG and 1.5 mol % DSPE. Since both PEG-DSG and PEG-DSPE utilize C18 lipid side chains, these studies demonstrated that the C18 PEG-Lipids in the LNPs displayed improved tumor:liver expression profiles post systemic administration of the LNPs.

### Example 6

### Additional BH3 Multimer Construct

In this example, an additional BH3 multimer construct, which contains multiple copies of the Beclin BH3 domain, was prepared. This construct contains three copies of the Beclin BH3 domain, with intervening F2A linker peptides in between and thus has the structure: BeclinBH3-F2A-BeclinBH3-F2A-BeclinBH3, optionally with a v5 epitope tag at the C-terminus.

The amino acid sequence of construct without the epitope tag is shown below, wherein the BeclinBH3 sequences are in bold and the linker sequences are underlined:

(SEQ ID NO: 546)
MGTMENLSRRLKVTGDLFDIMSGSGVKQTLNFDLLKLAGDVESNPGP
GTMENLSRRLKVTGDLFDIMSGSGVKQTLNFDLLKLAGDVESNPGP
GTMENLSRRLKVTGDLFDIMS

The amino acid sequences of construct with the C-terminal v5 epitope tag is shown below, wherein the BeclinBH3 sequences are in bold and the linker sequences are underlined:

(SEQ ID NO: 547)
MGTMENLSRRLKVTGDLFDIMSGSGVKQTLNFDLLKLAGDVESNPGP
GTMENLSRRLKVTGDLFDIMSGSGVKQTLNFDLLKLAGDVESNPGP
GTMENLSRRLKVTGDLFDIMSGKPIPNPLLGLDST

Two different nucleotide sequences encoding the above BeclinBH3x3.F2A amino acid sequence (with v5 tag) are shown below:

(SEQ ID NO: 548)
ATGGGCACCATGGAGAATCTTAGCAGACGACTGAAAGTGACCGGCGATCT
GTTCGACATCATGAGCGGCAGCGGCGTGAAGCAGACCCTGAACTTTGACT
TGCTGAAGCTGGCCGGCGACGTGGAAAGCAACCCCGGACCCGGCACCATG
GAGAACCTGAGCCGGCGGCTGAAGGTGACCGGCGACTTGTTCGACATCAT
GAGCGGCAGCGGAGTGAAGCAGACTTTGAACTTCGACCTTCTGAAACTGG
CCGGCGATGTGGAGAGCAATCCAGGCCCGGGCACCATGGAGAATCTGAGC
AGAAGACTGAAGGTGACTGGCGACCTGTTCGACATTATGAGCGGCAAGCC
CATCCCCAACCCCCTGCTGGGTCTGGATAGCACC
(SEQ ID NO: 549)
ATGGGAACTATGGAGAACCTGTCGCGGAGGTTGAAAGTGACCGGCGACCT
GTTTGACATTATGTCCGGCTCCGGAGTGAAGCAGACCCTGAACTTCGACC
TTTTGAAGCTGGCCGGCGACGTGGAATCGAACCCAGGCCCTGGTACTATG
GAAAACCTCAGCAGACGCCTGAAAGTCACCGGAGATCTGTTCGACATCAT
GAGCGGATCCGGCGTGAAGCAAACTCTGAATTTCGACCTCCTGAAGCTTG
CGGGAGATGTGGAGTCAAACCCGGGGCCCGGTACCATGGAAAATCTGTCC
CGCCGGCTCAAGGTCACCGGGGACCTGTTCGATATCATGTCCGGGAAGCC
TATCCCCAACCCGCTGCTGGGACTCGACAGCACC

These nucleotide sequences are incorporated into modified mRNA constructs that contain a 5′ UTR having the sequence shown in SEQ ID NO: 72, a 3′ UTR having the sequence shown in SEQ ID NO: 73, a Cap 1 5′ Cap (7mG(5′)ppp(5′)NlmpNp), a poly A tail of 100 nucleotides and are fully modified with 1-methyl-pseudouridine (m1ψ).

These mmRNA constructs can be translated in cell free systems as described above and used in apoptosis assays as described above.

### Example 7

### Apoptosis of Hep3B Cells Treated with mRNA Encoding Self-Cleaving or Uncleavable Multimeric BH3 Domains

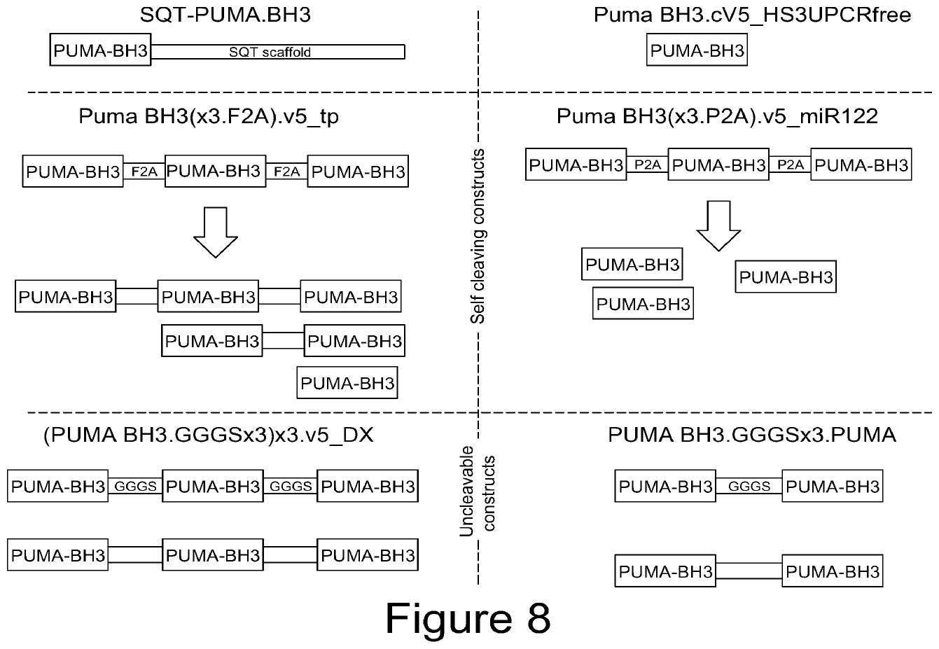

A series of multimeric BH3 domain constructs were prepared that contained either a self-cleaving linker (F2A or P2A) or an uncleavable linker (GGGS). The structure of these constructs is illustrated schematically in FIG. 8. The top row shows the SQT scaffolded positive control construct SQT-PUMA-BH3 and a BH3 monomer control construct. The middle row shows the self-cleaving constructs, including the final expression products produced after self-cleavage (shown after the arrow). F2A and P2A are self-cleaving linker peptides (F2A is 50% efficient, P2A is >90% efficient). The bottom row shows the uncleavable constructs, which remain as trimer or dimer expression products (shown after the arrow)

To determine whether the multimeric BH3 domain constructs containing either a self-cleaving linker or an uncleavable linker could induce apoptosis, Hep3B hepatocellular carcinoma cells (20,000 cells/well) were transfected using Lipofectamine 2000 (Life Technologies) with varying doses of the multimeric constructs shown in FIG. 8. As a positive control, an SQT-PUMA-BH3 mmRNA construct encoding a fusion polypeptide of the SQT scaffold linked to a single PUMA BH3 domain, which was known to induce apoptosis, was used. As a negative control, an “empty” SQT mmRNA construct, encoding only the SQT scaffold with no BH3 domain, was used. Cell death was measured by YOYO-3 (Life Technologies) staining after 24 hours.

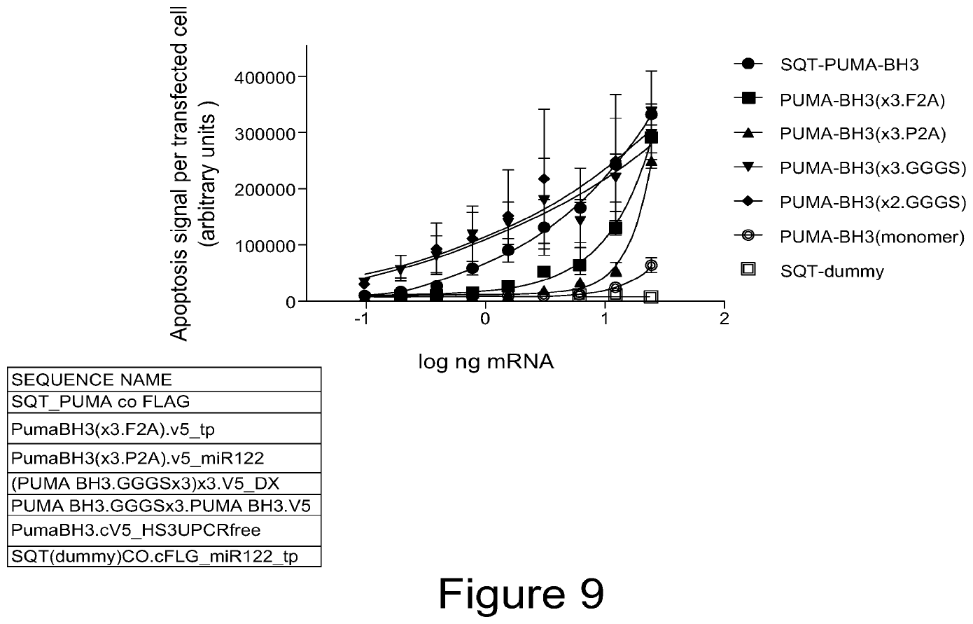

The results for apoptosis at 24 hours post-transfection are shown in the graph of FIG. 9. The results demonstrate that all mmRNA constructs encoding multimeric BH3 domains induced apoptosis of Hep3B cells, with the constructs containing uncleavable GGGS linkers ((PUMA BH3.GGGSx3)x3, encoding a trimer, and PUMA BH3.GGGSx3.PUMA, encoding a dimer) exhibiting higher potency than the constructs containing the self-cleaving linkers.

### Example 8

### Lipid Nanoparticle-Formulated Uncleavable Multimeric BH3 Construct Induces Apoptosis in Hep3B Cells

The efficacy of an mmRNA construct encoding uncleavable multimeric BH3 domains in inducing cell death was tested in the Hep3B hepatocellular carcinoma cell lines using lipid nanoparticle (LNP)-formulated constructs. Hep3B cells (10,000 cells/well) were treated with varying doses of SM86-formulated LNPs containing an mmRNA construct encoding (PUMA BH3.GGGSx3)x3, which encodes a BH3 domain trimer as illustrated schematically in FIG. 8. As a positive control, LNPs containing an SQT-PUMA-BH3 mmRNA construct, encoding a fusion polypeptide of the SQT scaffold linked to a single PUMA BH3 domain, which was known to induce apoptosis, was used. As negative controls, LNPs containing inactive NTFIX or GFP constructs were used. LNPs were applied to the cells in a 3-fold dilution series. Cell death was assessed using YOYO3 fluorescence imaging on Incucyte, followed by analysis of total integrated intensity of red fluorescence.

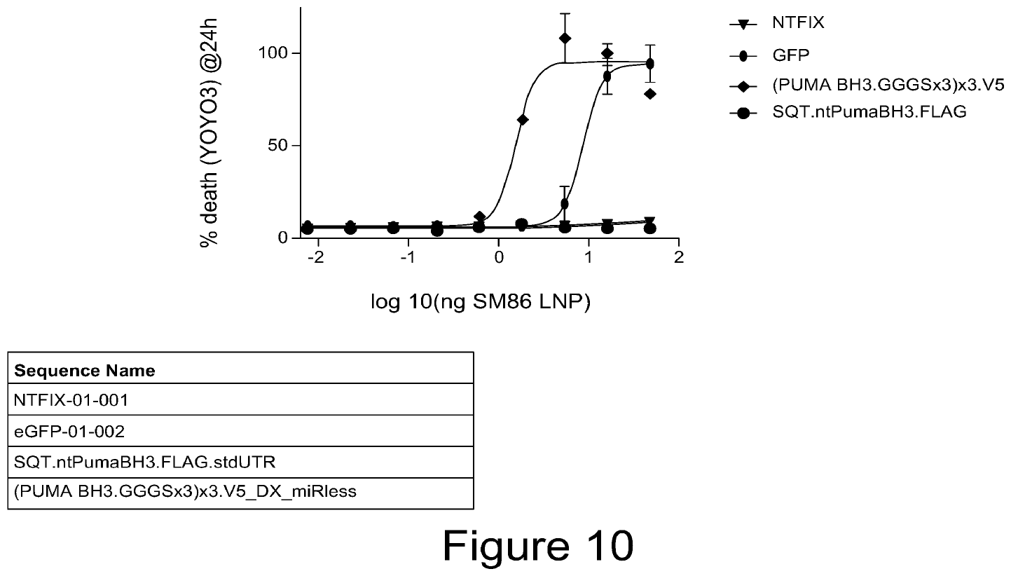

The results for apoptosis at 24 hours post-treatment are shown in the graph of FIG. 10. The results demonstrate that LNPs containing the mmRNA construct encoding the uncleavable multimeric BH3 domains induced apoptosis of Hep3B cells with a potency greater than the SQT-PUMA-BH3 positive control construct. The (PUMA BH3.GGGSx3)x3.V5 construct was reproducibly approximately 5-10 fold more potent than the SQT-PUMA-BH3 construct at inducing cell death.

### Example 9

### Preparation and Expression of mRNAs Encoding YAP Inhibitory Domains

A series of modified mRNA constructs were prepared that encoded one or more copies of selected YAP inhibitory domains from the VGLL4 protein on either a “scaffold” polypeptide (i.e., SQT) or as a multimer. For some constructs, a YAP inhibitory domain was inserted into the N-terminus of the Stefin A Quadruple Mutant-Tracy (SQT) variant of Stefin A, resulting in SQT-YAP inhibitory constructs. Multimer constructs contained multiple YAP inhibitory domains and a linker sequence (i.e., an F2A cleavable linker) between each YAP inhibitory domain. Thus, for example, a multimeric YAP inhibitory construct containing three anti-YAP domains with intervening F2A linkers has the structure: YAP inhibitory-F2A-YAP inhibitory-F2A-YAP inhibitory. The constructs also typically contained an epitope tag (i.e., v5) to facilitate detection of the encoded polypeptide(s). However, the skilled artisan will appreciate that constructs lacking the epitope tag, e.g., v5 tag) are preferable for therapeutic purposes. Some multimeric YAP inhibitory constructs contained F2A linkers between each YAP inhibitory domain and before the C-terminal epitope tag (e.g., having the structure: YAP inhibitory-F2A-YAP inhibitory-F2A-YAP inhibitory-F2A-affinity tag). In the multimer constructs, multiple copies of the same YAP inhibitory domain were used. The amino acid sequence encoded by the open reading frame (ORF) of each construct is shown below in Table 21.

TABLE 21
YAP inhibitory constructs

| mRNA Name(s) | ORF Amino Acid Sequence | SEQ |
|---|---|---|
| Super TDU | SVDDHFAKSLGDTWLQIGGSGNPKTANVPQTVPMRLRK | 463 |
| (NLS).cV5 | LPDSFFKPPEMTYPRRRFRRRRHRPRSHLGQILRRRPWL | |
| | VHPRHRYRWRRKNGIFNTGKPIPNPLLGLDST | |
| Super TDU | SVDDHFAKSLGDTWLQIGGSGNPKTANVPQTVPMRLRK | 464 |
| (sv40NLS).cV5 | LPDSFFKPPEPKKKRKVPKKKRKVPKKKRKVGIFNTGKP | |
| | IPNPLLGLDST | |
| MF2A(NLS).cV5 | SVDDHFAKSLGDTWLQIGGSGNPKTANVPQTVPARLRK | 465 |
| | LPDSAFKPPEMTYPRRRFRRRRHRPRSHLGQILRRRPWL | |
| | VHPRHRYRWRRKNGIFNTGKPIPNPLLGLDST | |
| MF2A(sv40NLS).cV5 | SVDDHFAKSLGDTWLQIGGSGNPKTANVPQTVPARLRK | 466 |
| | LPDSAFKPPEPKKKRKVPKKKRKVPKKKRKVGKPIPNPL | |
| | LGLDST | |
| HFMF4A(NLS).cV5 | SVDDAAAKSLGDTWLQIGGSGNPKTANVPQTVPARLRK | 467 |
| | LPDSAFKPPEMTYPRRRFRRRRHRPRSHLGQILRRRPWL | |
| | VHPRHRYRWRRKNGIFNTGKPIPNPLLGLDST | |
| HFMF4A(sv40NLS).c | SVDDAAAKSLGDTWLQIGGSGNPKTANVPQTVPARLRK | 468 |
| V5 | LPDSAFKPPEPKKKRKVPKKKRKVPKKKRKVGKPIPNPL | |
| | LGLDST | |
| huVGLL4- | STMGDPVVEEHFRRSLGKNYKEPEPAPNSVSITGSVDDH | 469 |
| TDU1.2(NLS).cV5 | FAKALGDTWLQIKAAKDSAMTYPRRRFRRRRHRPRSHL | |
| | GQILRRRPWLVHPRHRYRWRRKNGIFNTGKPIPNPLLGL | |
| | DST | |
| huVGLL4- | STMGDPVVEEHFRRSLGKNYKEPEPAPNSVSITGSVDDH | 470 |
| TDU1.2(sv40NLS).cV | FAKALGDTWLQIKAAKDSAPKKKRKVPKKKRKVPKKK | |
| 5 | RKVGKPIPNPLLGLDST | |
| huVGLL4.cV5 | METPLDVLSRAASLVHADDEKREAALRGEPRIQTLPVAS | 471 |
| | ALSSHRTGPPPISPSKRKFSMEPGDEDLDCDNDHVSKMS | |
| | RIFNPHLNKTANGDCRRDPRERSRSPIERAVAPTMSLHGS | |
| | HLYTSLPSLGLEQPLALTKNSLDASRPAGLSPTLTPGERQ | |
| | QNRPSVITCASAGARNCNLSHCPIAHSGCAAPGPASYRR | |
| | PPSAATTCDPVVEEHFRRSLGKNYKEPEPAPNSVSITGSV | |
| | DDHFAKALGDTWLQIKAAKDGASSSPESASRRGQPASPS | |
| | AHMVSHSHSPSVVSGKPIPNPLLGLDST | |
| huVGLL4(HF4A).cV5 | METPLDVLSRAASLVHADDEKREAALRGEPRIQTLPVAS | 472 |
| | ALSSHRTGPPPISPSKRKFSMEPGDEDLDCDNDHVSKMS | |
| | RIFNPHLNKTANGDCRRDPRERSRSPIERAVAPTMSLHGS | |
| | HLYTSLPSLGLEQPLALTKNSLDASRPAGLSPTLTPGERQ | |
| | QNRPSVITCASAGARNCNLSHCPIAHSGCAAPGPASYRR | |
| | PPSAATTCDPVVEEAARRSLGKNYKEPEPAPNSVSITGSV | |
| | DDAAAKALGDTWLQIKAAKDGASSSPESASRRGQPASP | |
| | SAHMVSHSHSPSVVSGKPIPNPLLGLDST | |
| SQT.(nt)VGLL4- | MIPRDPVVEEHFRRSLGKNYKEGLSEAKPATPEIQEIVDK | 473 |
| TDU1.(sv40NLS).cV5 | VKPQLEEKTNETYGKLEAVQYKTQVLASTNYYIKVRAG | |
| | DNKYMHLKVFNGPPGQNADRVLTGYQVDKNKDDELTG | |
| | FPKKKRKVPKKKKVPKKKRKVGKPIPNPLLGLDST | |
| SQT.(L1)VGLL4- | MIPRGLSEAKPATPEIQEIVDKVKPQLEEKTNETYGKLEA | 474 |
| TDU1.(sv40NLS).cV5 | VQYKTQVLDPVVEEHFRRSLGKNYKASTNYYIKVRAGD | |
| | NKYMHLKVFNGPPGQNADRVLTGYQVDKNKDDELTGF | |
| | PKKKRKVPKKKRVPKKKRKVGKPIPNPLLGLDST | |
| SQT.(L2)VGLL4- | MIPRGLSEAKPATPEIQEIVDKVKPQLEEKTNETYGKLEA | 475 |
| TDU1.(sv40NLS).cV5 | VQYKTQVLASTNYYIKVRAGDNKYMHLKVFNGPDPVV | |
| | EEHFRRSLGKNYKPGQNADRVLTGYQVDKNKDDELTGF | |
| | PKKKRKVPKKKRVPKKKRKVGKPIPNPLLGLDST | |
| SQT.(nt)VGLL4- | MIPRSVSITGSVDDHFAKALGDTWLQIKGLSEAKPATPEI | 476 |
| TDU2.(sv40NLS).cV5 | QEIVDKVKPQLEEKTNETYGKLEAVQYKTQVLASTNYYI | |
| | KVRAGDNKYMHLKVFNGPPGQNADRVLTGYQVDKNK | |
| | DDELTGFPKKKRVPKKKRKVPKKKRKVGKPIPNPLLGL | |
| | DST | |
| SQT.(L1)VGLL4- | MIPRGLSEAKPATPEIQEIVDKVKPQLEEKTNETYGKLEA | 477 |
| TDU2.(sv40NLS).cV5 | VQYKTQVLSVSITGSVDDHFAKALGDTWLQIKASTNYYI | |
| | KVRAGDNKYMHLKVFNGPPGQNADRVLTGYQVDKNK | |
| | DDELTGFPKKKRVPKKKRKVPKKKRKVGKPIPNPLLGL | |
| | DST | |
| SQT.(L2)VGLL4- | MIPRGLSEAKPATPEIQEIVDKVKPQLEEKTNETYGKLEA | 478 |
| TDU2.(sv40NLS).cV5 | VQYKTQVLASTNYYIKVRAGDNKYMHLKVFNGPSVSIT | |
| | GSVDDHFAKALGDTWLQIKPGQNADRVLTGYQVDKNK | |
| | DDELTGFPKKKRVPKKKRKVPKKKRKVGKPIPNPLLGL | |
| | DST | |
| huVGLL4- | STMGDPVVEEHFRRSLGKNYKEPEMTYPRRRFRRRRHR | 479 |
| TDU1(NLS).cV5 | PRSHLGQILRRRPWLVHPRHRYRWRRKNGIFNTGKPIPN | |
| | PLLGLDST | |
| huVGLL4- | STMSVSITGSVDDHFAKALGDTWLQIKAAKDSAMTYPR | 480 |
| TDU2(NLS).cV5 | RRFRRRRHRPRSHLGQILRRRPWLVHPRHRYRWRRKNG | |
| | IFNTGKPIPNPLLGLDST | |
| TandemPep.VGLL4.su | MSVDDHFAKSLGDTWLQIGSGVKQTLNFDLLKLAGDVE | 499 |
| perTDU | SNPGPSVDDHFAKSLGDTWLQIGSGVKQTLNFDLLKLA | |
| | GDVESNPGPSVDDHFAKSLGDTWLQIGKPIPNPLLGLDS | |
| | T | |
| TandemPep.VGLL4.T | MDPVVEEHFRRSLGKNYKEGSGVKQTLNFDLLKLAGDV | 500 |
| DU1 | ESNPGPDPVVEEHFRRSLGKNYKEGSGVKQTLNFDLLKL | |
| | AGDVESNPGPDPVVEEHFRRSLGKNYKEGKPIPNPLLGL | |
| | DST | |
| TandemPep.VGLL4.T | MSVSITGSVDDHFAKALGDTWLQIKGSGVKQTLNFDLL | 501 |
| DU2 | KLAGDVESNPGPSVSITGSVDDHFAKALGDTWLQIKGSG | |
| | VKQTLNFDLLKLAGDVESNPGPSVSITGSVDDHFAKALG | |
| | DTWLQIKGKPIPNPLLGLDST | |
| TandemPep.HFMF4A | MSVDDAAAKSLGDTWLQIGGSGNPKTANVPQTVPARLR | 502 |
| | KLPDSAFKPPEGSGVKQTLNFDLLKLAGDVESNPGPSVD | |
| | DAAAKSLGDTWLQIGGSGNPKTANVPQTVPARLRKLPD | |
| | SAFKPPEGSGVKQTLNFDLLKLAGDVESNPGPSVDDAA | |
| | AKSLGDTWLQIGGSGNPKTANVPQTVPARLRKLPDSAF | |
| | KPPEGKPIPNPLLGLDST | |
| TandemPep.MF2A | MSVDDHFAKSLGDTWLQIGGSGNPKTANVPQTVPARLR | 503 |
| | KLPDSAFKPPEGSGVKQTLNFDLLKLAGDVESNPGPSVD | |
| | DHFAKSLGDTWLQIGGSGNPKTANVPQTVPARLRKLPD | |
| | SAFKPPEGSGVKQTLNFDLLKLAGDVESNPGPSVDDHFA | |
| | KSLGDTWLQIGGSGNPKTANVPQTVPARLRKLPDSAFKP | |
| | PEGKPIPNPLLGLDST | |

The modified mRNA multimer constructs contained a 5′ UTR having the sequence shown in SEQ ID NO: 72 and a 3′ UTR having the sequence shown in SEQ ID NO: 73. The open reading frame (ORF) amino acid sequence shown in Table 21 encoded by the mRNA constructs include a v5 epitope tag sequence incorporated into the C-terminus of the sequence. This v5 epitope tag has the amino acid sequence: GKPIPNPLLGLDST (SEQ ID NO: 545). YAP inhibitory mRNA constructs having the open reading frames shown above in Table 21 but lacking the v5 epitope tag can also be prepared simply by deletion of the amino acids GKPIPNPLLGLDST (SEQ ID NO: 545) at the C-terminus of the sequences shown in Table 21.

Amino acid sequences corresponding to the constructs of SEQ ID NOs: 463-480 and 499-503 but without the epitope tag are shown in SEQ ID NOs: 481-498 and 504-507. Nucleotide sequences encoding the amino acid sequences shown in SEQ ID NOs: 463-480 and 499-503 are shown in SEQ ID NOs: 508-544.

### Example 10

### Induction of Apoptosis by Transfection of HGC27 and A549 Cells with mRNA Encoding YAP Inhibitory Domains

To determine whether YAP inhibitory domain constructs could induce apoptosis, HGC27 metastatic lymphoma cells and A549 lung carcinoma cells were used, which express high levels of YAP1.

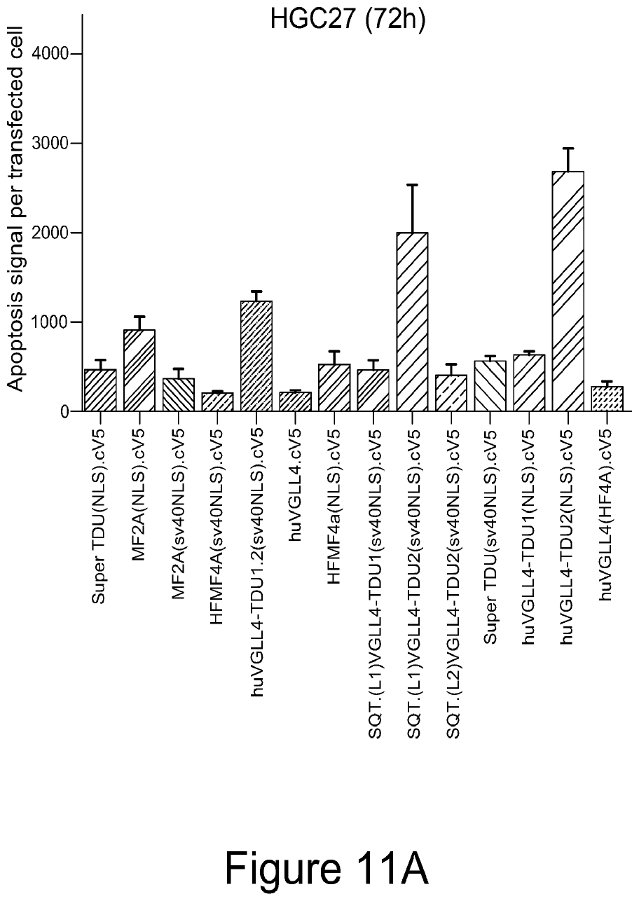

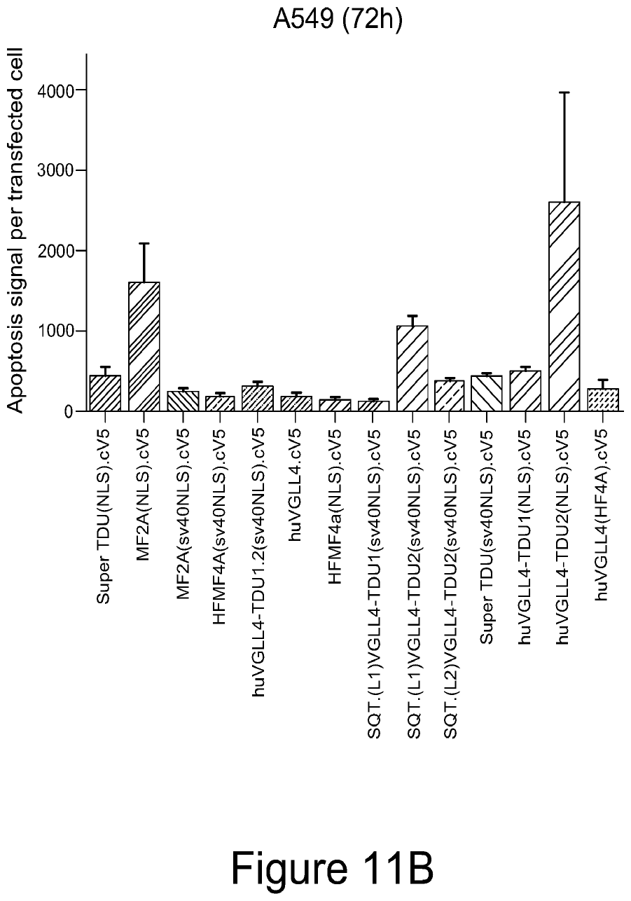

10,000 cells/well were plated in 96 well plates and the cells were transfected with 50 ng/100 mL of the different mRNA constructs using Lipofectamine 2000 (Life Technologies). Transfection was done in the presence of YOYO-3 (Life Technologies), a DNA dye that is taken up preferentially by dead cells that is used to measure the extent of cell death. The results for apoptosis at 72 hours post-transfection are shown in FIG. 11A for HGC27 cells and FIG. 11B for A549 cells. The results demonstrate that several YAP inhibitory constructs are cytotoxic to cell lines with high levels of YAP1. Constructs MF2A(NLS).cV5, huVGLL4-TDU1.2(sv40NLS).cV5, SQT.(L1)VGLL4-TDU2(sv40NLS).cV5, huVGLL4-TDU2(NLS).cV5 induced the highest levels of apoptosis in both cell lines.

Live images were taken over the 72 hours using Incucyte (Essen Bioscience). These images showed the uptake of YOYO-3 and confirmed that these constructs induced apoptosis. Overall, these results indicated that several YAP inhibitory constructs were capable of inducing apoptosis in metastatic lymphoma cells and in lung carcinoma cells.

### Example 11

### Binding of Anti-YAP1 Constructs to TEAD Transcription Factors

To determine whether YAP inhibitory constructs were binding with TEAD transcription factors, 2×10⁶ HGC27 cells were plated and transfected with 7.2 ug mRNA using Lipofectamine 2000. Cells were harvested after 6 hours and protein lysates were prepared using Pierce IP Lysis buffer (Thermo Fisher). Immunoprecipitation was performed using anti-V5 antibody (Life Technologies) against the epitope tag contained within the YAP inhibitory constructs. Immunoprecipitated samples were then run on a 4-12% Bis-Tris gel (Life Technologies) and transferred onto a 0.2 mm nitrocellulose membrane. For Western blot analysis, the membrane was probed with anti-TEAD4 antibody (Abcam) followed by HRP Rat Anti-Mouse Igk, Light Chain antibody (BD Pharmingen).

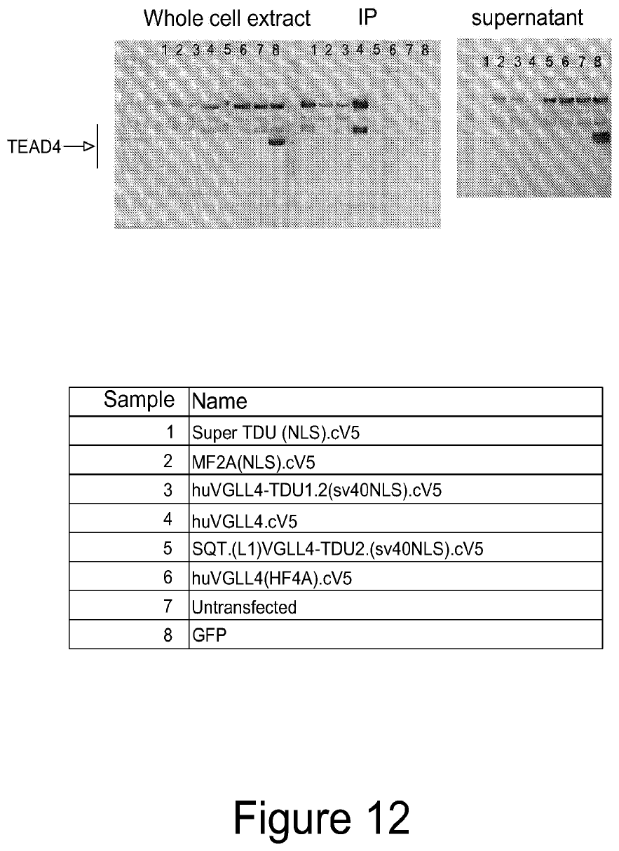

FIG. 12 shows the results of the Western blot analysis, wherein five out of six YAP inhibitory constructs were demonstrated to bind to TEAD4. These results indicated that transfection of the YAP inhibitory mRNA constructs into cells led to translation of the YAP inhibitory protein constructs and these protein constructs were capable of binding to a transcription factor (TEAD4) known to be regulated by YAP.

### Example 12

### Downregulation of YAP-Target Genes by Transfection of HGC27 Cells with mRNA Encoding YAP Inhibitory Domains

To determine whether YAP inhibitory constructs that bind to TEAD4 effect downstream YAP target genes (i.e., CTGF and CYR61), HGC27 cells were utilized. Specifically, 24 hours after 100,000 cells per well of a 24-well plate were seeded, each well was transfected with 250 ng of CMV-YAP1-puro plasmid with 0.5 μL Lipofectamine 2000 (Life Technologies). YAP1-containing cells were selected using 1 g/mL puromycin (Gibco) for 48 hours. Puromycin-selected cells were transfected with 250 g of YAP inhibitory mRNA using 1 uL Lipofectamine 2000. As a positive control, YAP-CMV plasmid was used. As a negative control, eGFP mRNA was used. 48 hours after transfection, RNA was extracted using the RNeasy kit (Qiagen) and cDNA was prepared using the High-Capacity cDNA Reverse Transcription Kit (ABI). qPCR reaction was performed using the TaqMan Fast Advanced Master Mix Reagent with CTGF (Hs01026927_g1) and/or CYR61 (Hs00998500_g1), and PPIA endogenous control (4326316E) Taqman probes (Life Technologies).

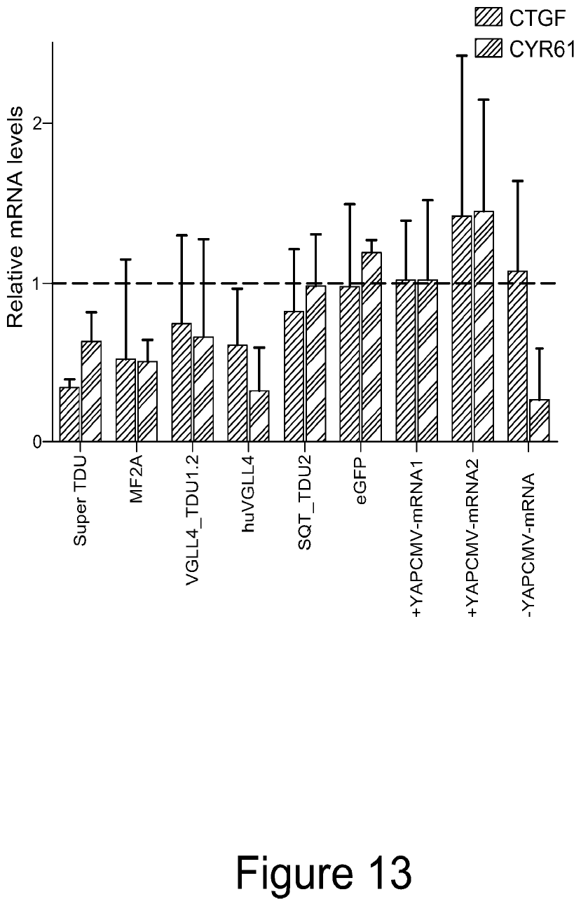

FIG. 13 shows the four YAP inhibitory constructs tested, which were shown to bind TEAD4, downregulated CTGF and CYR61. These results indicated that the YAP inhibitory mRNA constructs that bind TEAD4 have downstream effects on the YAP pathway.

### Example 13

### Induction of Apoptosis by Transfection of NCI-N87 Cells with mRNA Encoding YAP Inhibitory Domains

To determine whether the YAP inhibitory constructs capable of inducing apoptosis in HGC27 and A549 cells, could also induce apoptosis in another cell type, NCI-N87 gastric carcinoma cells were used. Specifically, 20,000 cells/well of a 96-well plate were transfected with 50 ng mRNA with Lipofectamine 2000. As a positive control, an SQT-PUMA-BH3 mRNA construct encoding a fusion polypeptide of the SQT scaffold linked to a single PUMA BH3 domain, which was known to induce apoptosis, was used.

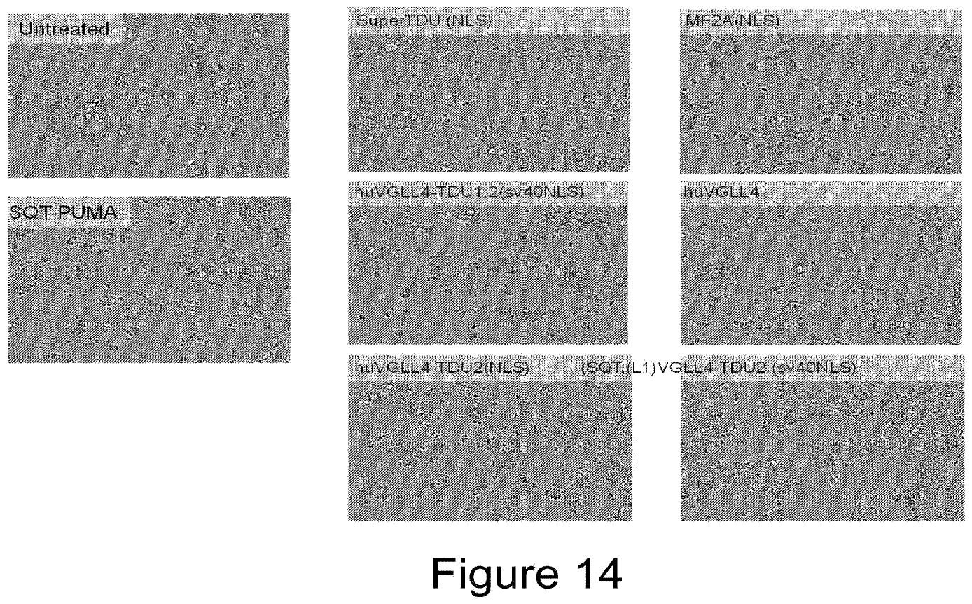

Images were taken 72 hours after transfection using Incucyte. The images showed the uptake of YOYO-3, which confirmed five out of thirteen constructs induced apoptosis (FIG. 14). These results demonstrate that YAP inhibitory mRNA constructs were capable of inducing apoptosis in a variety of cancer cells.

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

All references described herein are incorporated by reference in their entireties.

SEQUENCE LISTING SUMMARY

| SEQ | |
|---|---|
| ID | |
| NO: | SEQUENCE |
| 1 | LALRLACIGDEMDVS (BH3 domain amino acid sequence) |
| 2 | EKAELLQGGDLLRQR (BH3 domain amino acid sequence) |
| 3 | VESILKKNSDWIWDW (BH3 domain amino acid sequence) |
| 4 | EVEALKKSADWVSDW (BH3 domain amino acid sequence) |
| 5 | TAARLKALGDELHQR (BH3 domain amino acid sequence) |
| 6 | IAQELRRIGDEFNAY (BH3 domain amino acid sequence) |
| 7 | YGRELRRMSDEFVDS (BH3 domain amino acid sequence) |
| 8 | IARHLAQVGDSMDRS (BH3 domain amino acid sequence) |
| 9 | IARKLQCIADQFHRL (BH3 domain amino acid sequence) |
| 10 | CATQLRRFGDKLNFR (BH3 domain amino acid sequence) |
| 11 | IGAQLRRMADDLNAQ (BH3 domain amino acid sequence) |
| 12 | LSRRLKVTGDLFDIM (BH3 domain amino acid sequence) |
| 13 | IVELLKYSGDQLERK (BH3 domain amino acid sequence) |
| 14 | ALETLRRVGDGVQRN (BH3 domain amino acid sequence) |
| 15 | IGSKLAAMCDDFDAQ (BH3 domain amino acid sequence) |
| 16 | IGRKLTVMCDEFDSE (BH3 domain amino acid sequence) |
| 17 | NIRRLRALADGVQKV (BH3 domain amino acid sequence) |
| 18 | NIDKLRALADDIDKT (BH3 domain amino acid sequence) |
| 19 | MVTLLPIEGQEIHFF (BH3 domain amino acid sequence) |
| 20 | PTVPLPSETDGYVAP (BH3 domain amino acid sequence) |
| 21 | PQRYLVIQGDDRMKL (BH3 domain amino acid sequence) |
| 22 | TVGELSRALGHENGS (BH3 domain amino acid sequence) |
| 23 | VGQLLQDMGDDVYQQ (BH3 domain amino acid sequence) |
| 24 | LHEVLNGLLDRPDWE (BH3 domain amino acid sequence) |
| 25 | AVHSLSRIGDELYLE (BH3 domain amino acid sequence) |
| 26 | NPKFLKNAGRDCSRR (BH3 domain amino acid sequence) |
| 27 | EEQWAREIGAQLRRMADDLNAQYERR (BH3 domain amino acid sequence) |
| 28 | DMRPEIWIAQELRRIGDEFNAYYARR (BH3 domain amino acid sequence) |
| 29 | NLWAAQRYGRELRRMSDEFVDSFKKG (BH3 domain amino acid sequence) |
| 30 | PAELEVECATQLRRFGKLNFRQKLL (BH3 domain amino acid sequence) |
| 31 | UAUUUAGUGUGAUAAUGGCGUU (miR-122 sequence) |
| 32 | CAAACACCAUUGUCACACUCCA (miR-122 sequence) |
| 33 | UAGCUUAUCAGACUGAUGUUGA (miR-21 sequence) |
| 34 | CAACACCAGUCGAUGGGCUGU (miR-21 sequence) |
| 35 | GSGATNFSLLKQAGDVEENPGP (2A peptide amino acid sequence) |
| 36 | GGAAGCGGAGCTACTAACTTCAGCCTGCTGAAGCAGGCTGGAGACGTGG |
| | AGGAGAACCCTGGACCT (2A peptide encoding sequence) |
| 37 | TCCGGACTCAGATCCGGGGATCTCAAAATTGTCGCTCCTGTCAAACAAAC |
| | TCTTAACTTTGATTTACTCAAACTGGCTGGGGATGTAGAAAGCAATCCAG |
| | GTCCACTC (2A peptide encoding sequence) |
| 38 | MAHAGRTGYDNREIVMKYIHYKLSQRGYEWDAGDVGAAPPGAAPAP |
| | GIFSSQPGHTPHPAASRDPVARTSPLQTPAAPGAAAGPALSPVPPVVHL |
| | TLRQAGDDFSRRYRRDFAEMSSQLHLTPFTARGRFATVVEELFRDGVN |
| | WGRIVAFFEFGGVMCVESVNREMSPLVDNIALWMTEYLNRHLHTWIQ |
| | DNGGWDAFVELYGPSMRPLFDFSWLSLKTLLSLALVGACITLGAYLGHK |
| | (Human Bcl-2 amino acid sequence, Genbank NP_000624) |
| 39 | MSQSNRELVVDFLSYKLSQKGYSWSQFSDVEENRTEAPEGTESEMETPSA |
| | INGNPSWHLADSPAVNGATGHSSSLDAREVIPMAAVKQALREAGDEFELR |
| | YRRAFSDLTSQLHITPGTAYQSFEQVVNELFRDGVNWGRIVAFFSFGGAL |
| | CVESVDKEMQVLVSRIAAWMATYLNDHLEPWIQENGGWDTFVELYGNN |
| | AAAESRKGQERFNRWFLTGMTVAGVVLLGSLFSRK |
| | (Human Bcl-X_{L} amino acid sequence, Genbank NP_612815.1) |
| 40 | MATPASAPDTRALVADFVGYKLRQKGYVCGAGPGEGPAADPLHQAMRA |
| | AGDEFETRFRRTFSDLAAQLHVTPGSAQQRFTQVSDELFQGGPNWGRLVA |
| | FFVFGAALCAESVNKEMEPLVGQVQEWMVAYLETRLADWIHSSGGWAEF |
| | TALYGDGALEEARRLREGNWASVRTVLTGAVALGALVTVGAFFASK |
| | (Human Bcl-w amino acid sequence, Genbank NP_004041.1) |
| 41 | MFGLKRNAVIGLNLYCGGAGLGAGSGGATRPGGRLLATEKEASARREIGG |
| | GEAGAVIGGSAGASPPSTLTPDSRRVARPPPIGAEVPDVTATPARLLFFAPTR |
| | RAAPLEEMEAPAADAIMSPEEELDGYEPEPLGKRPAVLPLLELVGESGNNT |
| | STDGSLPSTPPPAEEEEDELYRQSLEIISRYLREQATGAKDTKPMGRSGATSR |
| | KALETLRRVGDGVQRNHETAFQGMLRKLDIKNEDDVKSLSRVMIHVFSDGV |
| | TNWGRIVTLISFGAFVAKHLKTINQESCIEPLAESITDVLVRTKRDWLVKQRG |
| | WDGFVEFFHVEDLEGGIRNVLLAFAGVAG VGAGLAYLIR |
| | (Human Mcl-1 amino acid sequence, Genbank NP_068779.1) |
| 42 | MTDCEFGYIYRLAQDYLQCVLQIPQPGSGPSKTSRVLQNVAFSVQKEVEK |
| | NLKSCLDNVNVVSVDTARTLFNQVMEKEFEDGIINWGRIVTIFAFEGILIKK |
| | LLRQQIAPDVDTYKEISYFVAEFIMNNTGEWIRQNGGWENGFVKKFEPKSG |
| | WMTFLEVTGKICEMLSLLKQYC |
| | (Human Bcl-related protein A1 amino acid sequence, Genbank NP_004040.1) |
| 43 | ATGATACCCAGACAAGAGGACATCATTCGTAACATCGCGCGACACCTGGC |
| | CCAGGTGGGGGACTCCATGGATCGATCAATCCCCCCTGGCTTATCCGAAG |
| | CTAAGCCAGCCACCCCTGAGATCCAAGAAATCGTCGACAAGGTCAAGCCC |
| | CAACTTGAAGAGAAAACTAATGAAACTTATGGTAAGCTCGAAGCCGTGCA |
| | ATACAAGACTCAGGTCCTGGCGTCTACAAATTATTACATCAAGGTGAGGG |
| | CCGGGGACAATAAGTATATGCATCTGAAAGTCTTCAATGGCCCCCCTGGA |
| | CAAAACGCAGATCGGGTGCTCACAGGATACCAGGTCGATAAAAATAAAG |
| | ATGACGAACTTACTGGGTTTGATTATAAAGATGATGATGACAAG |
| | (SQT(nt)BID-BH3 mRNA sequence with FLAG epitope tag) |
| 44 | ATGATTCCTCGGGGTTTGTCAGAGGCAAAGCCAGCCACCCCAGAAATACA |
| | GGAAATCGTAGACAAAGTGAAGCCCCAGCTTGAGGAAAAAACTAACGAG |
| | ACCTACGGGAAGCTGGAAGCTGTGCAGTACAAGACCCAGGTGCTACAGG |
| | AGGACATCATTAGGAATATTGCGAGACATCTGGCGCAAGTGGGTGACTCT |
| | ATGGATCGTTCCATTCCACCGGGTGCAAGCACCAATTATTACATCAAAGT |
| | GCGTGCTGGAGACAACAAATATATGCACCTCAAGGTTTTTAACGGGCCGC |
| | CAGGACAGAATGCAGACCGTGTGCTTACCGGGTATCAGGTAGACAAAAA |
| | TAAAGATGACGAGCTGACTGGCTTTGACTACAAGGATGACGATGACAAG |
| | (SQT(L1)BID-BH3 mRNA sequence with FLAG epitope tag) |
| 45 | ATGATCCCCAGGGGACTCTCCGAGGCAAAGCCAGCCACACCTGAAATTCA |
| | GGAAATCGTTGATAAAGTAAAGCCACAGCTTGAGGAAAAAACCAATGAG |
| | ACGTACGGCAAACTCGAAGCCGTGCAGTACAAGACCCAGGTATTAGCCTC |
| | CACCAACTACTACATAAAGGTGCGGGCCGGCGACAACAAGTACATGCATC |
| | TGAAAGTATTCAACGGTCCCCAGGAAGACATTATAAGGAACATTGCCCGC |
| | CATCTTGCTCAGGTGGGCGATTCCATGGACCGTAGCATACCTCCTGGGCA |
| | GAACGCCGATCGCGTACTCACGGGCTATCAAGTCGATAAAAACAAGGAT |
| | GATGAATTGACTGGGTTTGATTACAAAGATGATGACGATAAG |
| | (SQT(L2)BID-BH3 mRNA sequence with FLAG epitope tag) |
| 46 | ATGATACCCAGACAAGAGGACATCATTCGTAACATCGCGCGACACCTGGC |
| | CCAGGTGGGGGACTCCATGGATCGATCAATCCCCCCTGGCTTATCCGAAG |
| | CTAAGCCAGCCACCCCTGAGATCCAAGAAATCGTCGACAAGGTCAAGCCC |
| | CAACTTGAAGAGAAAACTAATGAAACTTATGGTAAGCTCGAAGCCGTGCA |
| | ATACAAGACTCAGGTCCTGGCGTCTACAAATTATTACATCAAGGTGAGGG |
| | CCGGGGACAATAAGTATATGCATCTGAAAGTCTTCAATGGCCCCCCTGGA |
| | CAAAACGCAGATCGGGTGCTCACAGGATACCAGGTCGATAAAAATAAAG |
| | ATGACGAACTTACTGGGTTT |
| | (SQT(nt)BID-BH3 mRNA sequence without epitope tag) |
| 47 | ATGATTCCTCGGGGTTTGTCAGAGGCAAAGCCAGCCACCCCAGAAATACA |
| | GGAAATCGTAGACAAAGTGAAGCCCCAGCTTGAGGAAAAAACTAACGAG |
| | ACCTACGGGAAGCTGGAAGCTGTGCAGTACAAGACCCAGGTGCTACAGG |
| | AGGACATCATTAGGAATATTGCGAGACATCTGGCGCAAGTGGGTGACTCT |
| | ATGGATCGTTCCATTCCACCGGGTGCAAGCACCAATTATTACATCAAAGT |
| | GCGTGCTGGAGACAACAAATATATGCACCTCAAGGTTTTTAACGGGCCGC |
| | CAGGACAGAATGCAGACCGTGTGCTTACCGGGTATCAGGTAGACAAAAA |
| | TAAAGATGACGAGCTGACTGGCTTT |
| | (SQT(L1)BID-BH3 mRNA sequence without epitope tag) |
| 48 | ATGATCCCCAGGGGACTCTCCGAGGCAAAGCCAGCCACACCTGAAATTCA |
| | GGAAATCGTTGATAAAGTAAAGCCACAGCTTGAGGAAAAAACCAATGAG |
| | ACGTACGGCAAACTCGAAGCCGTGCAGTACAAGACCCAGGTATTAGCCTC |
| | CACCAACTACTACATAAAGGTGCGGGCCGGCGACAACAAGTACATGCATC |
| | TGAAAGTATTCAACGGTCCCCAGGAAGACATTATAAGGAACATTGCCCGC |
| | CATCTTGCTCAGGTGGGCGATTCCATGGACCGTAGCATACCTCCTGGGCA |
| | GAACGCCGATCGCGTACTCACGGGCTATCAAGTCGATAAAAACAAGGAT |
| | GATGAATTGACTGGGTTT |
| | (SQT(L2)BID-BH3 mRNA sequence without epitope tag) |
| 49 | ATGATCCCAAGAGAAGAACAGTGGGCACGCGAAATTGGAGCTCAACTCA |
| | GACGGATGGCCGACGACCTGAACGCCCAGTACGAGCGGCGGGGACTCTC |
| | AGAAGCGAAGCCTGCCACCCCCGAAATCCAGGAAATCGTCGACAAGGTC |
| | AAGCCGCAGCTTGAGGAAAAGACCAACGAGACTTACGGAAAACTGGAGG |
| | CCGTGCAGTATAAGACCCAAGTGTTGGCCTCCACCAACTACTACATTAAG |
| | GTCCGCGCCGGCGATAACAAGTACATGCACCTGAAGGTGTTCAATGGTCC |
| | TCCGGGACAGAACGCGGACAGGGTGCTGACTGGCTACCAAGTGGACAAG |
| | AACAAGGACGACGAGCTGACGGGCTTCGACTACAAGGATGATGATGACA |
| | AA |
| | (SQT-PUMA-BH3 mRNA sequence with FLAG epitope tag) |
| 50 | ATGATCCCAAGAGACATGCGCCCCGAAATTTGGATCGCCCAGGAGCTCAG |
| | ACGCATCGGAGATGAATTCAACGCATACTACGCTCGGCGGGGACTCTCAG |
| | AAGCGAAGCCTGCCACCCCCGAAATCCAGGAAATCGTCGACAAGGTCAA |
| | GCCGCAGCTTGAGGAAAAGACCAACGAGACTTACGGAAAACTGGAGGCC |
| | GTGCAGTATAAGACCCAAGTGTTGGCCTCCACCAACTACTACATTAAGGT |
| | CCGCGCCGGCGATAACAAGTACATGCACCTGAAGGTGTTCAATGGTCCTC |
| | CGGGACAGAACGCGGACAGGGTGCTGACTGGCTACCAAGTGGACAAGAA |
| | CAAGGACGACGAGCTGACGGGCTTCGACTACAAGGATGATGATGACAAA |
| | (SQT-BIM-BH3 mRNA sequence with FLAG epitope tag) |
| 51 | ATGATCCCAAGAAACCTGTGGGCTGCACAACGCTACGGAAGAGAACTGC |
| | GCCGGATGAGCGACGAATTTGTGGACTCGTTCAAGAAGGGGGGACTCTCA |
| | GAAGCGAAGCCTGCCACCCCCGAAATCCAGGAAATCGTCGACAAGGTCA |
| | AGCCGCAGCTTGAGGAAAAGACCAACGAGACTTACGGAAAACTGGAGGC |
| | CGTGCAGTATAAGACCCAAGTGTTGGCCTCCACCAACTACTACATTAAGG |
| | TCCGCGCCGGCGATAACAAGTACATGCACCTGAAGGTGTTCAATGGTCCT |
| | CCGGGACAGAACGCGGACAGGGTGCTGACTGGCTACCAAGTGGACAAGA |
| | ACAAGGACGACGAGCTGACGGGCTTCGACTACAAGGATGATGATGACAA |
| | A (SQT-BAD-BH3 mRNA sequence with FLAG epitope tag) |
| 52 | ATGATCCCAAGACCCGCAGAGCTCGAAGTGGAATGCGCTACCCAACTTAG |
| | ACGGTTCGGAGACAAGCTGAACTTTCGGCAGAAGCTCCTGGGACTCTCAG |
| | AAGCGAAGCCTGCCACCCCCGAAATCCAGGAAATCGTCGACAAGGTCAA |
| | GCCGCAGCTTGAGGAAAAGACCAACGAGACTTACGGAAAACTGGAGGCC |
| | GTGCAGTATAAGACCCAAGTGTTGGCCTCCACCAACTACTACATTAAGGT |
| | CCGCGCCGGCGATAACAAGTACATGCACCTGAAGGTGTTCAATGGTCCTC |
| | CGGGACAGAACGCGGACAGGGTGCTGACTGGCTACCAAGTGGACAAGAA |
| | CAAGGACGACGAGCTGACGGGCTTCGACTACAAGGATGATGATGACAAA |
| | (SQT-NoxA-BH3 mRNA sequence with FLAG epitope tag) |
| 53 | MIPGGLSEAKPATPEIQEIVDKVKPQLEEKTNETYGKLEAVQYKTQVVAGTN |
| | YYIKVRAGDNKYMHLKVFKSLPGQNEDLVLTGYQVDKNKDDELTGF |
| | (WT SteA scaffold amino acid sequence) |
| 54 | MIPWGLSEAKPATPEIQEIVDKVKPQLEEKTNETYGKLEAVQYKTQVDAGTN |
| | YYIKVRAGDNKYMHLKVFNGPPGQNEDLVLTGYQVDKNKDDELTGF |
| | (SteA scaffold (STM) amino acid sequence) |
| 55 | MIPGGLSEAKPATPEIQEIVDKVKPQLEEKTNETYGKLEAVQYKTQVLASTNY |
| | YIKVRAGDNKYMHLKVFNGPPGQNEDLVRSGYQVDKNKDDELTGF |
| | (SteA scaffold (SDM) amino acid sequence) |
| 56 | MIPRGLSEAKPATPEIQEIVDKVKPQLEEKTNETYGKLEAVQYKTQVLASTNY |
| | YIKVRAGDNKYMHLKVFNGPPGQNEDLVRSGYQVDKNKDDELTGF |
| | (SteA scaffold (SQM) amino acid sequence) |
| 57 | MIPGGLSEAKPATPEIQEIVDKVKPQLEEKTNETYGKLEAVQYKTQVVAGTN |
| | YYIKVRAGDNKYMHLKVF NGPPGQNEDLVRSGYQVDKNKDDELTGF |
| | (SteA scaffold (SUC) amino acid sequence) |
| 58 | MIPGGLSEAKPATPEIQEIVDKVKPQLEEKTNETYGKLEAVQYKTQVLASTNY |
| | YIKVRAGDNKYMHLKVFKSLPGQNEDLVLTGYQVDKNKDDELTGF |
| | (SteA scaffold (SUM) amino acid sequence) |
| 59 | MIPRGLSEAKPATPEIQEIVDKVKPQLEEKTNETYGKLEAVQYKTQVVAGTN |
| | YYIKVRAGDNKYMHLKVFKSLPGQNEDLVLTGYQVDKNKDDELTGF |
| | (SteA scaffold (SUN) amino acid sequence) |
| 60 | MIPGGLSEAKPATPEIQEIVDKVKPQLEEKTNETYGKLEAVQYKTQVLASTNY |
| | YIKVRAGDNKYMHLKVFNGPPGQNEDLVRS |
| | (SteA scaffold (SDM-) amino acid sequence) |
| 61 | MIPGGLSEAKPATPEIQEIVDKVKPQLEEKTNETYGKLEAVQYKTQVLASTNY |
| | YIKVRAGDNKYMHLKVFNGP (SteA scaffold (SDM--) amino acid sequence) |
| 62 | MIPRGLSEAKPATPEIQEIVDKVKPQLEEKTNETYGKLEAVQYKTQVLASTNY |
| | YIKVRAGDNKYMHLKVFNGPPGQNEDLVRS |
| | (SteA scaffold (SQM-) amino acid sequence) |
| 63 | MIPRGLSEAKPATPEIQEIVDKVKPQLEEKTNETYGKLEAVQYKTQVLASTNY |
| | YIKVRAGDNKYMHLKVFNGP (SteA scaffold (SQM--) amino acid sequence) |
| 64 | MIPGGLSEAKPATPEIQEIVDKVKPQLEEKTNETYGKLEAVQYKTQVVAGTN |
| | YYIKVRAGDNKYMHLKVFNGPPGQNEDLVRS |
| | (SteA scaffold (SUC-) amino acid sequence) |
| 65 | MIPGGLSEAKPATPEIQEIVDKVKPQLEEKTNETYGKLEAVQYKTQVVAGTN |
| | YYIKVRAGDNKYMHLKVFNGP (SteA scaffold (SUC--) amino acid sequence) |
| 66 | MIPGGLSEAKPATPEIQEIVDKVKPQLEEKTNETYGKLEAVQYKTQVLASTNY |
| | YIKVRAGDNKYMHLKVFKSLPGQNEDLVLT |
| | (SteA scaffold (SUM-) amino acid sequence) |
| 67 | MIPGGLSEAKPATPEIQEIVDKVKPQLEEKTNETYGKLEAVQYKTQVLASTNY |
| | YIKVRAGDNKYMHLKVFKSL (SteA scaffold (SUM--) amino acid sequence) |
| 68 | MIPRGLSEAKPATPEIQEIVDKVKPQLEEKTNETYGKLEAVQYKTQVVAGTN |
| | YYIKVRAGDNKYMHLKVFKSLPGQNEDLVLT |
| | (SteA scaffold (SUN-) amino acid sequence) |
| 69 | MIPRGLSEAKPATPEIQEIVDKVKPQLEEKTNETYGKLEAVQYKTQVVAGTN |
| | YYIKVRAGDNKYMHLKVFKSL (SteA scaffold (SUN--) amino acid sequence) |
| 70 | MIPRGLSEAKPATPEIQEIVDKVKPQLEEKTNETYGKLEAVQYKTQVLASTNY |
| | YIKVRAGDNKYMHLKVFNGPPGQNADRVLTGYQVDKNKDDELTGF |
| | (SteA scaffold (SQT) amino acid sequence) |
| 71 | MIPRGLSEAKPATPEIQEIVDKVKPQLEEKTNETYGKLEAVQYKTQVLALAST |
| | NYYIKVRAGDNKYMHLKVFNGPPGQNADRVLTGYQVDKNKDDELTGF |
| | (SteA scaffold (SQL) amino acid sequence) |
| 72 | TCAAGCTTTTGGACCCTCGTACAGAAGCTAATACGACTCACTATAGGGAA |
| | ATAAGAGAGAAAAGAAGAGTAAGAAGAAATATAAGAGCCACC |
| | (5′ UTR) |
| 73 | TGATAATAGGCTGGAGCCTCGGTGGCCATGCTTCTTGCCCCTTGGGCCTCC |
| | CCCCAGCCCCTCCTCCCCTTCCTGCACCCGTACCCCCGTGGTCTTTGAATA |
| | AAGTCTGAGTGGGCGGC |
| | (3′ UTR) |
| 74 | ATGATCCCAAGAGAAGAACAGTGGGCACGCGAAATTGGAGCTCAACTCA |
| | GACGGATGGCCGACGACCTGAACGCCCAGTACGAGCGGCGGGGACTCTC |
| | AGAAGCGAAGCCTGCCACCCCCGAAATCCAGGAAATCGTCGACAAGGTC |
| | AAGCCGCAGCTTGAGGAAAAGACCAACGAGACTTACGGAAAACTGGAGG |
| | CCGTGCAGTATAAGACCCAAGTGTTGGCCTCCACCAACTACTACATTAAG |
| | GTCCGCGCCGGCGATAACAAGTACATGCACCTGAAGGTGTTCAATGGTCC |
| | TCCGGGACAGAACGCGGACAGGGTGCTGACTGGCTACCAAGTGGACAAG |
| | AACAAGGACGACGAGCTGACGGGCTTC |
| | (SQT-PUMA-BH3 mRNA sequence without epitope tag) |
| 75 | ATGATCCCAAGAGACATGCGCCCCGAAATTTGGATCGCCCAGGAGCTCAG |
| | ACGCATCGGAGATGAATTCAACGCATACTACGCTCGGCGGGGACTCTCAG |
| | AAGCGAAGCCTGCCACCCCCGAAATCCAGGAAATCGTCGACAAGGTCAA |
| | GCCGCAGCTTGAGGAAAAGACCAACGAGACTTACGGAAAACTGGAGGCC |
| | GTGCAGTATAAGACCCAAGTGTTGGCCTCCACCAACTACTACATTAAGGT |
| | CCGCGCCGGCGATAACAAGTACATGCACCTGAAGGTGTTCAATGGTCCTC |
| | CGGGACAGAACGCGGACAGGGTGCTGACTGGCTACCAAGTGGACAAGAA |
| | CAAGGACGACGAGCTGACGGGCTTC |
| | (SQT-BIM-BH3 mRNA sequence without epitope tag) |
| 76 | ATGATCCCAAGAAACCTGTGGGCTGCACAACGCTACGGAAGAGAACTGC |
| | GCCGGATGAGCGACGAATTTGTGGACTCGTTCAAGAAGGGGGGACTCTCA |
| | GAAGCGAAGCCTGCCACCCCCGAAATCCAGGAAATCGTCGACAAGGTCA |
| | AGCCGCAGCTTGAGGAAAAGACCAACGAGACTTACGGAAAACTGGAGGC |
| | CGTGCAGTATAAGACCCAAGTGTTGGCCTCCACCAACTACTACATTAAGG |
| | TCCGCGCCGGCGATAACAAGTACATGCACCTGAAGGTGTTCAATGGTCCT |
| | CCGGGACAGAACGCGGACAGGGTGCTGACTGGCTACCAAGTGGACAAGA |
| | ACAAGGACGACGAGCTGACGGGCTTC |
| | (SQT-BAD-BH3 mRNA sequence without epitope tag) |
| 77 | ATGATCCCAAGACCCGCAGAGCTCGAAGTGGAATGCGCTACCCAACTTAG |
| | ACGGTTCGGAGACAAGCTGAACTTTCGGCAGAAGCTCCTGGGACTCTCAG |
| | AAGCGAAGCCTGCCACCCCCGAAATCCAGGAAATCGTCGACAAGGTCAA |
| | GCCGCAGCTTGAGGAAAAGACCAACGAGACTTACGGAAAACTGGAGGCC |
| | GTGCAGTATAAGACCCAAGTGTTGGCCTCCACCAACTACTACATTAAGGT |
| | CCGCGCCGGCGATAACAAGTACATGCACCTGAAGGTGTTCAATGGTCCTC |
| | CGGGACAGAACGCGGACAGGGTGCTGACTGGCTACCAAGTGGACAAGAA |
| | CAAGGACGACGAGCTGACGGGCTTC |
| | (SQT-NoxA-BH3 mRNA sequence without epitope tag) |
| 78 | ATGATTCCTAATCCTCTCTTAGGGCTGGACGCAACACCCGCGTCCGCACCC |
| | GATACAAGAGCACTGGTTGCAGACTTCGTGGGCTACAAACTGAGGCAGA |
| | AGGGGTACGTCTGTGGTGCGGGCCCCGGTGAAGGGCCTGCCGCCGACCCT |
| | CTGCACCAGGCTATGCGAGCTGCAGGGGACGAGTTTGAGACACGCTTTAG |
| | ACGTACATTTTCTGATTTGGCGGCTCAGCTCCACGTCACCCCTGGGAGCGC |
| | TCAGCAGCGATTTACCCAAGTAAGCGACGAGCTCTTTCAAGGAGGCCCCA |
| | ATTGGGGCCGGCTGGTCGCGTTCTTTGTGTTTGGGGCCGCATTATGCGCGG |
| | AAAGCGTTAACAAAGAGATGGAACCCTTAGTAGGACAAGTCCAGGAATG |
| | GATGGTGGCCTACCTAGAGACACAGCTAGCTGACTGGATTCATTCATCAG |
| | GGGGCTGGGCCGAGTTTACTGCCTTATATGGGGATGGGGCACTCGAAGAA |
| | GCCAGGAGACTTCGCGAAGGGAATTGGGCATCAGTGAGGACTGTGCTAA |
| | CTGGAGCCGTCGCCCTGGGTGCCCTGGTTACCGTGGGTGCTTTCTTTGCGT |
| | CTAAA (Bcl-w wild-type mRNA sequence with V5 epitope tag) |
| 79 | ATGATTCCTAACCCCCTACTAGGACTTGACTCTCAGAGCAACAGAGAATT |
| | GGTAGTGGATTTTCTGAGTTATAAACTATCGCAGAAAGGCTACTCTTGGTC |
| | ACAGTTCTCAGATGTTGAGGAGAACAGGACTGAAGCACCAGAGGGGACA |
| | GAGTCTGAGATGGAGACCCCCAGTGCTATAAACGGTAACCCCTCCTGGCA |
| | CCTAGCCGATAGCCCCGCCGTGAACGGAGCCACCGGACACAGCAGCTCCT |
| | TAGATGCCCGCGAGGTGATTCCGATGGCCGCAGTAAAGCAGGCTCTCCGA |
| | GAAGCCGGCGATGAATTTGAGCTTAGGTATAGGAGGGCCTTCTCTGATCT |
| | TACTTCACAGCTTCACATCACACCTGGCACAGCTTATCAGAGCTTCGAAC |
| | AGGTGGTGAATGAACTTTTTCGAGATGGCGTAAACTGGGGCCGCATCGTG |
| | GCCTTCTTTAGCTTCGGTGGGGCCCTGTGCGTCGAATCAGTGGACAAAGA |
| | AATGCAAGTCCTGGTGAGCCGAATCGCAGCCTGGATGGCCACGTACTTGA |
| | ACGATCACCTGGAGCCTTGGATCCAAGAGAACGGCGGATGGGACACTTTC |
| | GTCGAGTTGTATGGCAATAATGCCGCTGCCGAAAGTCGGAAAGGGCAGG |
| | AGCGATTTAACCGCTGGTTCCTTACAGGCATGACTGTTGCTGGGGTGGTG |
| | CTCTTGGGTTCTCTCTTCAGCCGCAAA |
| | (Bcl-xL wild-type mRNA sequence with V5 epitope tag) |
| 80 | ATGGGACCACTCGGGTCGGAGGATGACCTGTATCGCCAATCCCTGGAAAT |
| | CATCTCTAGGTACCTGAGGGAGCAGGCCACCGGCTCGAAAGATAGTAAAC |
| | CACTTGGCGAAGCCGGAGCTGCTGGCAGAAGGGCTCTCGAGACACTCCGG |
| | CGCGTGGGTGATGGAGTGCAACGCAATCACGAGACTGCCTTCCAGGGCAT |
| | GCTCAGGAAGCTGGATATCAAAAACGAGGATGATGTTAAGAGTCTCTCTC |
| | GAGTCATGATTCATGTGTTTTCCGACGGTGTCACTAACTGGGGTAGAATTG |
| | TTACTCTGATAAGTTTTGGAGCATTCGTTGCCAAGCACTTGAAGACAATTA |
| | ACCAGGAGTCGTGCATCGAGCCCCTAGCGGAAAGCATCACAGATGTTCTC |
| | GTGCGAACCAAGCGCGATTGGCTGGTCAAGCAGAGGGGATGGGACGGGT |
| | TTGTTGAGTTTTTCCACGTCGAGGATCTGGAAGGGGGGGACTACAAGGAT |
| | GATGACGACAAG (Mcl-1 del.N/C mRNA sequence with FLAG epitope tag) |
| 81 | ATGATTCCCAATCCGCTCCTCGGGCTTGACGCTACACCAGCCAGTGCACC |
| | AGACACGAGGGCGCTGGTTGCCGATTTCGTGGGATACAAGCTCAGGCAAA |
| | AAGGGTACGTAAGCGGTGCCGGGCCCGGGGAGGGACCGGCTGCCGATCC |
| | ACTCCACCAGGCTATGAGGGCAGCTGGGGACGAATTCGAAACACGCTTTC |
| | GGCGGACTTTCTCCGACCTTGCCGCACAGCTACACGTCACTCCCGGGAGC |
| | GCTCAGCAGCGCTTCACGCAGGTCTCAGACGAATTGTTCCAAGGCGGCCC |
| | TAATTGGGGAAGATTGGTGGCCTTCTTCGTGTTTGGGGCAGCACTGTGCG |
| | CTGAGTCCGTGAACAAAGAAATGGAACCCCTCGTTGGCCAAGTCCAAGAA |
| | TGGATGGTGGAATATCTGGAAACACAGTTAGCGGATTGGATTCACTCATC |
| | TGGGGGCTGGGCTGAGTTTACAGCACTGTACGGGGACGGTGCCCTAGAGG |
| | AGGCCCGAAGACTGAGGGAAGGCAACTGGGCCAGCGTTCGCACGGTGTT |
| | GACCGGCGCGGTTGCTTTGGGCGCTTTGGTTACCGTCGGCGCATTCTTCGC |
| | CAGTAAA (Bcl-w (C29S/A128E)BH3 mRNA sequence with V5 epitope tag) |
| 82 | ATGGCACACGCTGGTAGGACAGGCTACGACAACCGTGAGATTGTGATGA |
| | AGTATATTCACTATAAGCTGTCTCAAAGAGGCTACGAATGGGATGCGGGG |
| | GCCGTCGGGGCTGCCCCACCTGGTGCCGCCCCGGCTCCTGGGATTTTCTCT |
| | TCCCAGCCGGGACATACCCCTCATCCTGCCGCTTCTAGGGACCCTGTGGC |
| | AAGAACATCCCCTCTACAGACCCCTGCCGCCCCAGGTGCGGCTGCTGGCC |
| | CAGCGTTAAGTCCCGTTCCACCAGTCGTGCACCTTACCCTTAGGCAAGC |
| | GGGCGACGATTTCTCTAGGCGATACCGGAGGGACTTTGCCGAAATGTCAT |
| | CACAGCTCCATCTTACTCCATTTACTGCCCGTGGCAGATTCGCCACCGTAG |
| | TGGAAGAACTCTTTCGAGACGGCGTAAATTGGGGGAGGATCGTGGCATTT |
| | TTCGAATTTGGAGGGGTTATGTGTGTGGAGAGCGTGAATCGGGAAATGTC |
| | TCCTCTGGTTGACAATATTGCCTTGTGGATGACCGAATATCTTAACAGGCA |
| | CCTCCACACCTGGATTCAAGATAATGGTGGATGGGATGCGTTTGTGGAA |
| | CTTTATGGGCCTTCAATGCGTGACTATAAGGACGACGACGACAAA |
| | (Bcl-2(D34A)del.C32 BH3 mRNA sequence with FLAG epitope tag) |
| 83 | ATGTCCCAGAGCAACAGGGAACTCGTAGTCGATTTCTTGTCCTACAAGCT |
| | CAGTCAGAAGGGCTACTCTTGGTCCCAATTTTCCGACGTGGAAGAAAATC |
| | GGACAGAAGCCCCTGAGGGAACCGAGAGTGAGATGGAGACCCCGTCTGC |
| | AATCAACGGGAACCCTTCTTGGCACTTGGCCGACAGTCCCGCTGTGAATG |
| | GCGCCACAGGTCACAGTTCATCACTCGACGCTAGAGAAGTTATACCCATG |
| | GCAGCAGTCAAGCAAGCTCTGCGGGAGGCCGGCGACGAGTTCGAACTTC |
| | GGTACCGCCGAGCTTTTTCTGACCTGACTAGTCAGCTGCATATTACTCCGG |
| | GCACTGCGTACCAGTCGTTCGAACAGGTGGTTAATGAACTATTTCGAGAC |
| | GGCGTGAATTGGGGCAGAATTGTAGCCTTTTTCTCCTTTGGAGGTGCACTG |
| | TGTGTCGAGAGCGTCGATAAGGAAATGCAGGTGTTGGTGAGCCGTATCGC |
| | GGCCTGGATGGCCACGTATCTGAACGACCATTTGGAACCATGGATCCAAG |
| | AAAACGGGGGTTGGGATACTTTCGTGGAGTTATATGGCAACAATGCTGCA |
| | GCGGAAAGTCGCAAGGGCCAGGAACGAGACTATAAAGATGACGACGATA |
| | AA (Bcl-xL del.C24 mRNA sequence with FLAG epitope tag) |
| 84 | ATGGGCTCTGATGAGCTGTACCGGCAGAGCCTCGAGATCATAAGCAGATA |
| | CCTGAGGGAACAAGCGACGGGGGCCAAGGATACTAAGCCCATGGGACGC |
| | TCCGGGGCAACGTCTCGCAAAGCCCTCGAGACCCTCAGAAGGGTCGGCGA |
| | TGGGGTACAGCGCAACCACGAGACTGCTTTCCAGGGCATGCTCCGAAAGC |
| | TGGACATCAAGAACGAAGACGATGTGAAGTCTCTTTCCCGTGTGATGATA |
| | CATGTGTTTTCTGATGGGGTTACCAACTGGGGACGCATTGTGACCCTTATT |
| | AGCTTCGGGGCGTTCGTCGCTAAGCACCTCAAGACAATCAATCAGGAAAG |
| | TTGTATTGAACCTTTAGCTGAGAGCATAACTGACGTCTTGGTCCGTACAAA |
| | GAGGGATTGGTTGGTCAAACAACGTGGTTGGGATGGTTTCGTAGAGTTCT |
| | TCCATGTCGAAGATCTTGAGGGAGGGGACTACAAAGATGACGATGATAA |
| | A (Mcl1 del.N/C(2010) mRNA sequence with FLAG epitope tag) |
| 85 | ATGAGCCAGTCTAACCGCGAACTTGTTGTGGACTTCCTTAGTTATAAACTC |
| | TCTCAGAAAGGGTATTCTTGGAGTCAGTTTTCTGATGTTGAAGAAAATCG |
| | GACTGAAGCTCCCGAGGGGACCGAAAGTGAGATGGAGACCCCAAGCGCA |
| | ATAAATGGCAACCCATCTTGGCATCTCGCCGCCTCTCCCGCAGTAAACGG |
| | AGCTACCGGTCACTCTTCTAGCCTGGACGCCCGGGAGGTGATACCCATGG |
| | CAGCCGTTAAGCAAGCCTTGCGGGAAGCAGGGGATGAGTTCGAACTTCGC |
| | TACAGGAGGGCATTTTCTGACCTCACCTCCCAGTTGCATATTACGCCAGG |
| | GACGGCATACCAGAGCTTTGAGCAAGTGGTCAATGAACTCTTTCGAGACG |
| | GAGTCAACTGGGGACGCATTGTAGCCTTCTTTTCATTTGGGGGCGCCCTGT |
| | GTGTGGAGTCTGTCGACAAGGAGATGCAGGTGCTCGTAAGCCGCATCGCC |
| | GCCTGGATGGCGACCTATCTTAATGATCACCTAGAACCTTGGATACAGGA |
| | GAATGGCGGATGGGACACCTTCGTTGAGCTGTACGGCAACAACGCGGCCG |
| | CAGAAAGCCGCAAGGGTCAAGAGAGGGATTATAAAGACGATGACGACAA |
| | A (Bcl-xL(D61A)del.C24 mRNA sequence with FLAG epitope tag) |
| 86 | ATGATTCCTCGAGGGCTATCAGAAGCTAAGCCTGCCACACCAGAGATTCA |
| | GGAGATCGTGGACAAGGTGAAGCCACAACTCGAGGAGAAAACCAACGAA |
| | ACATACGGCAAGCTCGAAGCTGTCCAGTATAAAACTCAGGTCTTAGATAT |
| | GCGACCAGAAATTTGGATTGCCCAGGAAGCTCGGCGCATCGGTGACGAG |
| | GCTAATGCCTATTATGCCAGAAGGGCAAGCACTAATTATTATATCAAAGT |
| | GCGAGCCGGAGATAATAAATACATGCACCTCAAGGTATTCAACGGACCCC |
| | CTGGGCAAAACGCTGACAGAGTCTTAACCGGATATCAGGTTGACAAAAAT |
| | AAGGACGACGAGCTTACCGGATTCGATTATAAAGATGATGACGACAAA |
| | (SQTanti-Mcl1.(L1)BimBH3.2A mRNA sequence with FLAG epitope tag) |
| 87 | ATGATACCTAGGGATATGAGGCCTGAGATTTGGATAGCACAGGAGGCAC |
| | GTAGGATAGGAGATGAAGCCAATGCATACTACGCCAGGCGCGGTCTGTCA |
| | GAGGCCAAACCGGCAACACCAGAGATCCAAGAAATTGTCGATAAAGTCA |
| | AGCCTCAGTTAGAGGAGAAGACTAATGAGACGTATGGCAAGCTCGAAGC |
| | AGTGCAATACAAGACTCAGGTCCTGGCTTCCACCAATTATTATATCAAAG |
| | TGCGCGCCGGCGACAACAAGTACATGCACCTTAAGGTTTTTAACGGCCCT |
| | CCAGGCCAGAATGCTGACCGTGTGCTGACAGGTTACCAGGTCGACAAGAA |
| | TAAGGACGATGAGTTGACCGGCTTCGACTATAAGGACGATGACGATAAA |
| | (SQTanti-Mcl1.(nt)BimBH3.2A mRNA sequence with FLAG epitope tag) |
| 88 | ATGATTCCTCGTGGCCTCTCTGAGGCCAAGCCTGCCACCCCCGAGATACA |
| | GGAAATCGTTGACAAAGTTAAACCTCAGCTCGAGGAAAAGACCAACGAG |
| | ACTTACGGAAAACTTGAGGCAGTGCAGTATAAGACTCAGGTCCTGAAAGC |
| | TCTCGAAACTCTGAGACGCGTGGGGGATGGTGTCCAGCGTAATCACGAGA |
| | CGGCCTTTGCAAGCACAAATTATTACATTAAGGTGCGAGCCGGCGATAAC |
| | AAATACATGCACCTCAAAGTGTTTAATGGACCGCCCGGCCAGAACGCCGA |
| | CCGCGTATTGACTGGGTACCAGGTAGACAAAAACAAGGATGACGAACTT |
| | ACAGGGTTCGACTACAAAGATGATGACGATAAG |
| | (SQT.(L1)Mcl1.BH3 mRNA sequence with FLAG epitope tag) |
| 89 | ATGATACCCCGGGGGCTTTCGGAGGCAAAGCCAGCTACCCCTGAAATCCA |
| | AGAAATCGTGGATAAGGTGAAACCTCAGCTGGAGGAGAAAACCAACGAA |
| | ACATACGGGAAACTGGAGGCAGTTCAATACAAAACACAGGTTTTGGCCA |
| | GCACTAATTATTACATCAAAGTGAGAGCGGGCGACAATAAGTATATGCAT |
| | TTGAAGGTGTTCAACGGGCCTAAGGCTCTCGAGACTCTACGGCGGGTCGG |
| | AGACGGCGTCCAGAGAAACCACGAGACTGCATTCCCTGGCCAGAACGCA |
| | GACCGTGTCCTGACTGGCTACCAGGTCGATAAAAACAAGGACGATGAGCT |
| | GACGGGATTCGACTATAAGGACGATGATGACAAA |
| | (SQT.(L2)Mcl1.BH3 mRNA sequence with FLAG epitope tag) |
| 90 | ATGATTCCAAGGGACATGAGACCAGAGATCTGGATTGCACAGGAGGCTA |
| | GGCGCATAGGAGATGAAGCAAATGCGTATTATGCACGACGCGGTCTTTCT |
| | GAAGCTAAGCCTGCGACGCCGGAGATTCAGGAGATAGTGGATAAAGTTA |
| | AACCTCAATTAGAGGAAAAGACCAATGAGACCTACGGCAAACTGGAAGC |
| | CGTGCAATATAAAACTCAGGTTCTGGCCTCCACTAACTATTATATCAAAGT |
| | GAGGGCAGGAGACAACAAGTACATGCACTTAAAGGTGTTCAATGGGCCC |
| | AACCTGTGGGCGGCCCAGCGCTACGGTCGCGAGCTACGAAGGATGTCTGA |
| | CGAGTTCGTTGACAGCTTCAAGAAGGGGCCTGGACAGAACGCCGATAGA |
| | GTGTTAACAGGCTATCAGGTCGATAAAAATAAAGATGACGAGCTAACCG |
| | GGTTTGATTACAAAGATGACGATGATAAG |
| | (biSQTanti-Bcl/Mcl1.(nt)BimBH2.2A.(L2)Bad mRNA sequence with FLAG |
| | epitope tag) |
| 91 | ATGATTCCCAGAGGCTTGAGTGAGGCTAAGCCCGCCACACCTGAGATCCA |
| | GGAAATCGTGGACAAGGTGAAACCCCAGCTGGAAGAAAAAACCAACGAG |
| | ACCTACGGCAAACTGGAGGCTGTCCAGTACAAAACTCAAGTCCTGGCCTC |
| | AACCAACTACTACATTAAAGTTAGAGCAGGCGATAATAAATACATGCACC |
| | TTAAAGTATTCAACGGTCCTGACATGCGACCTGAGATTTGGATAGCACAG |
| | GAAGCAAGAAGGATTGGGGATGAAGCTAATGCATATTACGCCCGCCGCC |
| | CGGGGCAGAATGCTGACCGTGTGCTGACCGGTTATCAGGTAGACAAGAAC |
| | AAAGACGACGAGCTAACAGGCTTCGACTACAAAGACGATGATGATAAG |
| | (SQTanti-Mcl1.(L2)BimBH3.2A mRNA sequence with FLAG epitope tag) |
| 92 | ATGGCAACACCCGCGTCCGCACCCGATACAAGAGCACTGGTTGCAGACTT |
| | CGTGGGCTACAAACTGAGGCAGAAGGGGTACGTCTGTGGTGCGGGCCCC |
| | GGTGAAGGGCCTGCCGCCGACCCTCTGCACCAGGCTATGCGAGCTGCAGG |
| | GGACGAGTTTGAGACACGCTTTAGACGTACATTTTCTGATTTGGCGGCTCA |
| | GCTCCACGTCACCCCTGGGAGCGCTCAGCAGCGATTTACCCAAGTAAGCG |
| | ACGAGCTCTTTCAAGGAGGCCCCAATTGGGGCCGGCTGGTCGCGTTCTTT |
| | GTGTTTGGGGCCGCATTATGCGCGGAAAGCGTTAACAAAGAGATGGA |
| | ACCCTTAGTAGGACAAGTCCAGGAATGGATGGTGGCCTACCTAGAGACAC |
| | AGCTAGCTGACTGGATTCATTCATCAGGGGGCTGGGCCGAGTTTACTGCC |
| | TTATATGGGGATGGGGCACTCGAAGAAGCCAGGAGACTTCGCGAAGGGA |
| | ATTGGGCATCAGTGAGGACTGTGCTAACTGGAGCCGTCGCCCTGGGTGCC |
| | CTGGTTACCGTGGGTGCTTTCTTTGCGTCTAAA |
| | (Bcl-w wild-type mRNA sequence without epitope tag) |
| 93 | ATGTCTCAGAGCAACAGAGAATTGGTAGTGGATTTTCTGAGTTATAAACT |
| | ATCGCAGAAAGGCTACTCTTGGTCACAGTTCTCAGATGTTGAGGAGAACA |
| | GGACTGAAGCACCAGAGGGGACAGAGTCTGAGATGGAGACCCCCAGTGC |
| | TATAAACGGTAACCCCTCCTGGCACCTAGCCGATAGCCCCGCCGTGAACG |
| | GAGCCACCGGACACAGCAGCTCCTTAGATGCCCGCGAGGTGATTCCGATG |
| | GCCGCAGTAAAGCAGGCTCTCCGAGAAGCCGGCGATGAATTTGAGCTTAG |
| | GTATAGGAGGGCCTTCTCTGATCTTACTTCACAGCTTCACATCACACCTG |
| | GCACAGCTTATCAGAGCTTCGAACAGGTGGTGAATGAACTTTTTCGAGAT |
| | GGCGTAAACTGGGGCCGCATCGTGGCCTTCTTTAGCTTCGGTGGGGCCCT |
| | GTGCGTCGAATCAGTGGACAAAGAAATGCAAGTCCTGGTGAGCCGAATC |
| | GCAGCCTGGATGGCCACGTACTTGAACGATCACCTGGAGCCTTGGATCCA |
| | AGAGAACGGCGGATGGGACACTTTCGTCGAGTTGTATGGCAATAATGCCG |
| | CTGCCGAAAGTCGGAAAGGGCAGGAGCGATTTAACCGCTGGTTCCTTACA |
| | GGCATGACTGTTGCTGGGGTGGTGCTCTTGGGTTCTCTCTTCAGCCGCAAA |
| | (Bcl-xL wild-type mRNA sequence without epitope tag) |
| 94 | ATGGGACCACTCGGGTCGGAGGATGACCTGTATCGCCAATCCCTGGAAAT |
| | CATCTCTAGGTACCTGAGGGAGCAGGCCACCGGCTCGAAAGATAGTAAAC |
| | CACTTGGCGAAGCCGGAGCTGCTGGCAGAAGGGCTCTCGAGACACTCCGG |
| | CGCGTGGGTGATGGAGTGCAACGCAATCACGAGACTGCCTTCCAGGGCAT |
| | GCTCAGGAAGCTGGATATCAAAAACGAGGATGATGTTAAGAGTCTCTCTC |
| | GAGTCATGATTCATGTGTTTTCCGACGGTGTCACTAACTGGGGTAGAATTG |
| | TTACTCTGATAAGTTTTGGAGCATTCGTTGCCAAGCACTTGAAGACAATTA |
| | ACCAGGAGTCGTGCATCGAGCCCCTAGCGGAAAGCATCACAGATGTTCTC |
| | GTGCGAACCAAGCGCGATTGGCTGGTCAAGCAGAGGGGATGGGACGGGT |
| | TTGTTGAGTTTTTCCACGTCGAGGATCTGGAAGGGGGG |
| | (Mcl-1 del.N/C mRNA sequence without epitope tag) |
| 95 | ATGGCTACACCAGCCAGTGCACCAGACACGAGGGCGCTGGTTGCCGATTT |
| | CGTGGGATACAAGCTCAGGCAAAAAGGGTACGTAAGCGGTGCCGGGCCC |
| | GGGGAGGGACCGGCTGCCGATCCACTCCACCAGGCTATGAGGGCAGCTG |
| | GGGACGAATTCGAAACACGCTTTCGGCGGACTTTCTCCGACCTTGCCGCA |
| | CAGCTACACGTCACTCCCGGGAGCGCTCAGCAGCGCTTCACGCAGGTCTC |
| | AGACGAATTGTTCCAAGGCGGCCCTAATTGGGGAAGATTGGTGGCCTTCT |
| | TCGTGTTTGGGGCAGCACTGTGCGCTGAGTCCGTGAACAAAGAAATGGAA |
| | CCCCTCGTTGGCCAAGTCCAAGAATGGATGGTGGAATATCTGGAAACACA |
| | GTTAGCGGATTGGATTCACTCATCTGGGGGCTGGGCTGAGTTTACAGCAC |
| | TGTACGGGGACGGTGCCCTAGAGGAGGCCCGAAGACTGAGGGAAGGCAA |
| | CTGGGCCAGCGTTCGCACGGTGTTGACCGGCGCGGTTGCTTTGGGCGCTTT |
| | GGTTACCGTCGGCGCATTCTTCGCCAGTAAA |
| | (Bcl-w (C29S/A128E)BH3 mRNA sequence without epitope tag) |
| 96 | ATGGCACACGCTGGTAGGACAGGCTACGACAACCGTGAGATTGTGATGA |
| | AGTATATTCACTATAAGCTGTCTCAAAGAGGCTACGAATGGGATGCGGGG |
| | GCCGTCGGGGCTGCCCCACCTGGTGCCGCCCCGGCTCCTGGGATTTTCTCT |
| | TCCCAGCCGGGACATACCCCTCATCCTGCCGCTTCTAGGGACCCTGTGGC |
| | AAGAACATCCCCTCTACAGACCCCTGCCGCCCCAGGTGCGGCTGCTGGCC |
| | CAGCGTTAAGTCCCGTTCCACCAGTCGTGCACCTTACCCTTAGGCAAGCG |
| | GGCGACGATTTCTCTAGGCGATACCGGAGGGACTTTGCCGAAATGT |
| | CATCACAGCTCCATCTTACTCCATTTACTGCCCGTGGCAGATTCGCCACCG |
| | TAGTGGAAGAACTCTTTCGAGACGGCGTAAATTGGGGGAGGATCGTGGCA |
| | TTTTTCGAATTTGGAGGGGTTATGTGTGTGGAGAGCGTGAATCGGGAAAT |
| | GTCTCCTCTGGTTGACAATATTGCCTTGTGGATGACCGAATATCTTAACAG |
| | GCACCTCCACACCTGGATTCAAGATAATGGTGGATGGGATGCGTTTGTGG |
| | AACTTTATGGGCCTTCAATGCGT |
| | (Bcl-2(D34A)del.C32 BH3 mRNA sequence without epitope tag) |
| 97 | ATGTCCCAGAGCAACAGGGAACTCGTAGTCGATTTCTTGTCCTACAAGCT |
| | CAGTCAGAAGGGCTACTCTTGGTCCCAATTTTCCGACGTGGAAGAAAATC |
| | GGACAGAAGCCCCTGAGGGAACCGAGAGTGAGATGGAGACCCCGTCTGC |
| | AATCAACGGGAACCCTTCTTGGCACTTGGCCGACAGTCCCGCTGTGAATG |
| | GCGCCACAGGTCACAGTTCATCACTCGACGCTAGAGAAGTTATACCCATG |
| | GCAGCAGTCAAGCAAGCTCTGCGGGAGGCCGGCGACGAGTTCGAACTTC |
| | GGTACCGCCGAGCTTTTTCTGACCTGACTAGTCAGCTGCATATTACTCC |
| | GGGCACTGCGTACCAGTCGTTCGAACAGGTGGTTAATGAACTATTTCGAG |
| | ACGGCGTGAATTGGGGCAGAATTGTAGCCTTTTTCTCCTTTGGAGGTGCAC |
| | TGTGTGTCGAGAGCGTCGATAAGGAAATGCAGGTGTTGGTGAGCCGTATC |
| | GCGGCCTGGATGGCCACGTATCTGAACGACCATTTGGAACCATGGATCCA |
| | AGAAAACGGGGGTTGGGATACTTTCGTGGAGTTATATGGCAACAATGCTG |
| | CAGCGGAAAGTCGCAAGGGCCAGGAACGA |
| | (Bcl-xL del.C24 mRNA sequence without epitope tag) |
| 98 | ATGGGCTCTGATGAGCTGTACCGGCAGAGCCTCGAGATCATAAGCAGATA |
| | CCTGAGGGAACAAGCGACGGGGGCCAAGGATACTAAGCCCATGGGACGC |
| | TCCGGGGCAACGTCTCGCAAAGCCCTCGAGACCCTCAGAAGGGTCGGCGA |
| | TGGGGTACAGCGCAACCACGAGACTGCTTTCCAGGGCATGCTCCGAAAGC |
| | TGGACATCAAGAACGAAGACGATGTGAAGTCTCTTTCCCGTGTGATGATA |
| | CATGTGTTTTCTGATGGGGTTACCAACTGGGGACGCATTGTGACCCTTATT |
| | AGCTTCGGGGCGTTCGTCGCTAAGCACCTCAAGACAATCAATCAGGAAAG |
| | TTGTATTGAACCTTTAGCTGAGAGCATAACTGACGTCTTGGTCCGTACAAA |
| | GAGGGATTGGTTGGTCAAACAACGTGGTTGGGATGGTTTCGTAGAGTTCT |
| | TCCATGTCGAAGATCTTGAGGGAGGG |
| | (Mcl1 del.N/C(2010) mRNA sequence without epitope tag) |
| 99 | ATGAGCCAGTCTAACCGCGAACTTGTTGTGGACTTCCTTAGTTATAAACTC |
| | TCTCAGAAAGGGTATTCTTGGAGTCAGTTTTCTGATGTTGAAGAAAATCG |
| | GACTGAAGCTCCCGAGGGGACCGAAAGTGAGATGGAGACCCCAAGCGCA |
| | ATAAATGGCAACCCATCTTGGCATCTCGCCGCCTCTCCCGCAGTAAACGG |
| | AGCTACCGGTCACTCTTCTAGCCTGGACGCCCGGGAGGTGATACCCATGG |
| | CAGCCGTTAAGCAAGCCTTGCGGGAAGCAGGGGATGAGTTCGAACTTCGC |
| | TACAGGAGGGCATTTTCTGACCTCACCTCCCAGTTGCATATTACGCCAGG |
| | GACGGCATACCAGAGCTTTGAGCAAGTGGTCAATGAACTCTTTCGAGACG |
| | GAGTCAACTGGGGACGCATTGTAGCCTTCTTTTCATTTGGGGGCGCCCTGT |
| | GTGTGGAGTCTGTCGACAAGGAGATGCAGGTGCTCGTAAGCCGCATCGCC |
| | GCCTGGATGGCGACCTATCTTAATGATCACCTAGAACCTTGGATACAGGA |
| | GAATGGCGGATGGGACACCTTCGTTGAGCTGTACGGCAACAACGCGGCCG |
| | CAGAAAGCCGCAAGGGTCAAGAGAGG |
| | (Bcl-xL(D61A)del.C24 mRNA sequence without epitope tag) |
| 100 | ATGATTCCTCGAGGGCTATCAGAAGCTAAGCCTGCCACACCAGAGATTCA |
| | GGAGATCGTGGACAAGGTGAAGCCACAACTCGAGGAGAAAACCAACGAA |
| | ACATACGGCAAGCTCGAAGCTGTCCAGTATAAAACTCAGGTCTTAGATAT |
| | GCGACCAGAAATTTGGATTGCCCAGGAAGCTCGGCGCATCGGTGACGAG |
| | GCTAATGCCTATTATGCCAGAAGGGCAAGCACTAATTATTATATCAAAGT |
| | GCGAGCCGGAGATAATAAATACATGCACCTCAAGGTATTCAACGGACCCC |
| | CTGGGCAAAACGCTGACAGAGTCTTAACCGGATATCAGGTTGACAAAAAT |
| | AAGGACGACGAGCTTACCGGATTC |
| | (SQTanti-Mcl1.(L1)BimBH3.2A mRNA sequence without epitope tag) |
| 101 | ATGATACCTAGGGATATGAGGCCTGAGATTTGGATAGCACAGGAGGCAC |
| | GTAGGATAGGAGATGAAGCCAATGCATACTACGCCAGGCGCGGTCTGTCA |
| | GAGGCCAAACCGGCAACACCAGAGATCCAAGAAATTGTCGATAAAGTCA |
| | AGCCTCAGTTAGAGGAGAAGACTAATGAGACGTATGGCAAGCTCGAAGC |
| | AGTGCAATACAAGACTCAGGTCCTGGCTTCCACCAATTATTATATCAAAG |
| | TGCGCGCCGGCGACAACAAGTACATGCACCTTAAGGTTTTTAACGGCCCT |
| | CCAGGCCAGAATGCTGACCGTGTGCTGACAGGTTACCAGGTCGACAAGAA |
| | TAAGGACGATGAGTTGACCGGCTTC |
| | (SQTanti-Mcl1.(nt)BimBH3.2A mRNA sequence without epitope tag) |
| 102 | ATGATTCCTCGTGGCCTCTCTGAGGCCAAGCCTGCCACCCCCGAGATACA |
| | GGAAATCGTTGACAAAGTTAAACCTCAGCTCGAGGAAAAGACCAACGAG |
| | ACTTACGGAAAACTTGAGGCAGTGCAGTATAAGACTCAGGTCCTGAAAGC |
| | TCTCGAAACTCTGAGACGCGTGGGGGATGGTGTCCAGCGTAATCACGAGA |
| | CGGCCTTTGCAAGCACAAATTATTACATTAAGGTGCGAGCCGGCGATAAC |
| | AAATACATGCACCTCAAAGTGTTTAATGGACCGCCCGGCCAGAACGCCGA |
| | CCGCGTATTGACTGGGTACCAGGTAGACAAAAACAAGGATGACGAACTT |
| | ACAGGGTTC (SQT.(L1)Mcl1.BH3 mRNA sequence without epitope tag) |
| 103 | ATGATACCCCGGGGGCTTTCGGAGGCAAAGCCAGCTACCCCTGAAATCCA |
| | AGAAATCGTGGATAAGGTGAAACCTCAGCTGGAGGAGAAAACCAACGAA |
| | ACATACGGGAAACTGGAGGCAGTTCAATACAAAACACAGGTTTTGGCCA |
| | GCACTAATTATTACATCAAAGTGAGAGCGGGCGACAATAAGTATATGCAT |
| | TTGAAGGTGTTCAACGGGCCTAAGGCTCTCGAGACTCTACGGCGGGTCGG |
| | AGACGGCGTCCAGAGAAACCACGAGACTGCATTCCCTGGCCAGAACGCA |
| | GACCGTGTCCTGACTGGCTACCAGGTCGATAAAAACAAGGACGATGAGCT |
| | GACGGGATTC (SQT.(L2)Mcl1.BH3 mRNA sequence without epitope tag) |
| 104 | ATGATTCCAAGGGACATGAGACCAGAGATCTGGATTGCACAGGAGGCTA |
| | GGCGCATAGGAGATGAAGCAAATGCGTATTATGCACGACGCGGTCTTTCT |
| | GAAGCTAAGCCTGCGACGCCGGAGATTCAGGAGATAGTGGATAAAGTTA |
| | AACCTCAATTAGAGGAAAAGACCAATGAGACCTACGGCAAACTGGAAGC |
| | CGTGCAATATAAAACTCAGGTTCTGGCCTCCACTAACTATTATATCAAAGT |
| | GAGGGCAGGAGACAACAAGTACATGCACTTAAAGGTGTTCAATGGGCCC |
| | AACCTGTGGGCGGCCCAGCGCTACGGTCGCGAGCTACGAAGGATGTCTGA |
| | CGAGTTCGTTGACAGCTTCAAGAAGGGGCCTGGACAGAACGCCGATAGA |
| | GTGTTAACAGGCTATCAGGTCGATAAAAATAAAGATGACGAGCTAACCG |
| | GGTTT |
| | (biSQTanti-Bcl/Mcl1.(nt)BimBH2.2A.(L2)Bad mRNA sequence without |
| | epitope tag) |
| 105 | ATGATTCCCAGAGGCTTGAGTGAGGCTAAGCCCGCCACACCTGAGATCCA |
| | GGAAATCGTGGACAAGGTGAAACCCCAGCTGGAAGAAAAAACCAACGAG |
| | ACCTACGGCAAACTGGAGGCTGTCCAGTACAAAACTCAAGTCCTGGCCTC |
| | AACCAACTACTACATTAAAGTTAGAGCAGGCGATAATAAATACATGCACC |
| | TTAAAGTATTCAACGGTCCTGACATGCACCTGAGATTTGGATAGCACAGG |
| | AAGCAAGAAGGATTGGGGATGAAGCTAATGCATATTACGCCCGCCGCCC |
| | GGGGCAGAATGCTGACCGTGTGCTGACCGGTTATCAGGTAGACAAGAAC |
| | AAAGACGACGAGCTAACAGGCTTC |
| | (SQTanti-Mcl1.(L2)BimBH3.2A mRNA sequence without epitope tag) |
| 106 | UCAACAUCAGUCUGAUAAGCUA |
| | (miR-21 sequence) |
| 107 | MIPRDMRPEIWIAQEARRIGDEANAYYARRGLSEAKPATPEIQEIVDKVKPQL |
| | EEKTNETYGKLEAVQYKTQVLASTNYYIKVRAGDNKYMHLKVFNGPPGQN |
| | ADRVLTGYQVDKNKDDELTGFGKPIPNPLLGLDST |
| | (SQTanti-Mcl1.(nt)BimBH3.2A.v5) |
| 108 | MDMRPEIWIAQEARRIGDEANAYYARRGSGVKQTLNFDLLKLAGDVESNPG |
| | PDMRPEIWIAQEARRIGDEANAYYARRGSGVKQTLNFDLLKLAGDVESNPGP |
| | DMRPEIWIAQEARRIGDEANAYYARRGKPIPNPLLGLDST |
| | (BimBH3.2A(x3.F2A).v5_miR122) |
| 109 | MIPRKALETLRRVGDGVQRNHETAFGLSEAKPATPEIQEIVDKVKPQLEEKTN |
| | ETYGKLEAVQYKTQVLASTNYYIKVRAGDNKYMHLKVFNGPPGQNADRVL |
| | TGYQVDKNKDDELTGFGKPIPNPLLGLDST |
| | (SQT.(nt)Mcl1.BH3.cv5) |
| 110 | MKALETLRRVGDGVQRNHETAFGSGVKQTLNFDLLKLAGDVESNPGPKALE |
| | TLRRVGDGVQRNHETAFGSGVKQTLNFDLLKLAGDVESNPGPKALETLRRV |
| | GDGVQRNHETAFGKPIPNPLLGLDST |
| | (Mcl1.BH3(x3.F2A).cV5) |
| 111 | MRPEIWMTQGLRRLGDEINAYYARGSGVKQTLNFDLLKLAGDVESNPGPRP |
| | EIWMTQGLRRLGDEINAYYARGSGVKQTLNFDLLKLAGDVESNPGPRPEIW |
| | MTQGLRRLGDEINAYYARGKPIPNPLLGLDST |
| | (antiMcl1.MS1(x3.F2A).cV5) |
| 112 | MRPEIWLTQSLQRLGDEINAYYARGSGVKQTLNFDLLKLAGDVESNPGPRPEI |
| | WLTQSLQRLGDEINAYYARGSGVKQTLNFDLLKLAGDVESNPGPRPEIWLTQ |
| | SLQRLGDEINAYYARGKPIPNPLLGLDST |
| | (antiMcl1.MS2(x3.F2A).cV5) |
| 113 | MRPEIWLTQHLQRLGDEINAYYARGSGVKQTLNFDLLKLAGDVESNPGPRPE |
| | IWLTQHLQRLGDEINAYYARGSGVKQTLNFDLLKLAGDVESNPGPRPEIWLT |
| | QHLQRLGDEINAYYARGKPIPNPLLGLDST |
| | (antiMcl1.MS3(x3.F2A).cV5) |
| 114 | MDFSVLQTIGDSLGSGVKQTLNFDLLKLAGDVESNPGPDFSVLQTIGDSLGSG |
| | VKQTLNFDLLKLAGDVESNPGPDFSVLQTIGDSLGKPIPNPLLGLDST |
| | (antiMcl1.SB-02(x3.F2A).cV5) |
| 115 | MNETVNTMLTYYYGSGVKQTLNFDLLKLAGDVESNPGPNETVNTMLTYYY |
| | GSGVKQTLNFDLLKLAGDVESNPGPNETVNTMLTYYYGKPIPNPLLGLDST |
| | (antiMcl1.SB-03(x3.F2A).cV5) |
| 116 | MNETVELMQAYLHGSGVKQTLNFDLLKLAGDVESNPGPNETVELMQAYLH |
| | GSGVKQTLNFDLLKLAGDVESNPGPNETVELMQAYLHGKPIPNPLLGLDST |
| | (antiMcl1.SB-04(x3.F2A).cV5) |
| 117 | MIPRDMRPEIWIAQEARRIGDEANAYYARRGLSEAKPATPEIQEIVDKVKPQL |
| | EEKTNETYGKLEAVQYKTQVLASTNYYIKVRAGDNKYMHLKVFNGPPGQN |
| | ADRVLTGYQVDKNKDDELTGF |
| | (SQTanti-Mcl1.(nt)BimBH3.2A without v5 tag) |
| 118 | MDMRPEIWIAQEARRIGDEANAYYARRGSGVKQTLNFDLLKLAGDVESNPG |
| | PDMRPEIWIAQEARRIGDEANAYYARRGSGVKQTLNFDLLKLAGDVESNPGP |
| | DMRPEIWIAQEARRIGDEANAYYARR |
| | (BimBH3.2A(x3.F2A)_miR122 without v5 tag) |
| 119 | MIPRKALETLRRVGDGVQRNHETAFGLSEAKPATPEIQEIVDKVKPQLEEKTN |
| | ETYGKLEAVQYKTQVLASTNYYIKVRAGDNKYMHLKVFNGPPGQNADRVL |
| | TGYQVDKNKDDELTGF (SQT.(nt)Mcl1.BH3 without v5 tag) |
| 120 | MKALETLRRVGDGVQRNHETAFGSGVKQTLNFDLLKLAGDVESNPGPKALE |
| | TLRRVGDGVQRNHETAFGSGVKQTLNFDLLKLAGDVESNPGPKALETLRRV |
| | GDGVQRNHETAF (Mcl1.BH3(x3.F2A) without v5 tag) |
| 121 | MRPEIWMTQGLRRLGDEINAYYARGSGVKQTLNFDLLKLAGDVESNPGPRP |
| | EIWMTQGLRRLGDEINAYYARGSGVKQTLNFDLLKLAGDVESNPGPRPEIW |
| | MTQGLRRLGDEINAYYAR |
| | (antiMcl1.MS1(x3.F2A) without v5 tag) |
| 122 | MRPEIWLTQSLQRLGDEINAYYARGSGVKQTLNFDLLKLAGDVESNPGPRPEI |
| | WLTQSLQRLGDEINAYYARGSGVKQTLNFDLLKLAGDVESNPGPRPEIWLTQ |
| | SLQRLGDEINAYYAR |
| | (antiMcl1.MS2(x3.F2A) without v5 tag) |
| 123 | MRPEIWLTQHLQRLGDEINAYYARGSGVKQTLNFDLLKLAGDVESNPGPRPE |
| | IWLTQHLQRLGDEINAYYARGSGVKQTLNFDLLKLAGDVESNPGPRPEIWLT |
| | QHLQRLGDEINAYYAR |
| | (antiMcl1.MS3(x3.F2A) without v5 tag) |
| 124 | MDFSVLQTIGDSLGSGVKQTLNFDLLKLAGDVESNPGPDFSVLQTIGDSLGSG |
| | VKQTLNFDLLKLAGDVESNPGPDFSVLQTIGDSL |
| | (antiMcl1.SB-02(x3.F2A) without v5 tag) |
| 125 | MNETVNTMLTYYYGSGVKQTLNFDLLKLAGDVESNPGPNETVNTMLTYYY |
| | GSGVKQTLNFDLLKLAGDVESNPGPNETVNTMLTYYY |
| | (antiMcl1.SB-03(x3.F2A) without v5 tag) |
| 126 | MNETVELMQAYLHGSGVKQTLNFDLLKLAGDVESNPGPNETVELMQAYLH |
| | GSGVKQTLNFDLLKLAGDVESNPGPNETVELMQAYLH |
| | (antiMcl1.SB-04(x3.F2A) without v5 tag) |
| 127 | ATGATCCCAAGAGATATGCGCCCTGAAATCTGGATTGCACAAGAAGCCCG |
| | GAGGATTGGAGATGAGGCGAACGCCTACTACGCCCGGAGAGGCCTGTCG |
| | GAAGCTAAACCGGCCACCCCCGAGATCCAGGAGATCGTGGATAAGGTCA |
| | AGCCCCAGCTTGAAGAAAAGACTAACGAAACCTATGGGAAGCTGGAGGC |
| | CGTGCAGTACAAGACTCAAGTGCTCGCGTCCACCAACTACTACATCAAGG |
| | TCCGCGCCGGCGACAACAAGTACATGCACTTGAAAGTGTTCAATGGCCCG |
| | CCGGGACAGAACGCCGACCGGGTGCTGACTGGATACCAGGTCGACAAGA |
| | ACAAGGACGACGAGCTGACCGGATTCGGGAAGCCTATTCCCAACCCTCTC |
| | CTGGGTCTGGACAGCACC |
| | (SQTanti-Mcl1.(nt)BimBH3.2A.v5) |
| 128 | ATGGACATGAGGCCCGAGATTTGGATCGCTCAGGAAGCACGCAGGATCG |
| | GCGACGAGGCCAACGCATACTACGCCCGGCGCGGCTCCGGAGTGAAGCA |
| | GACCCTGAATTTCGACTTGCTCAAGCTGGCCGGCGACGTGGAATCGAACC |
| | CAGGTCCCGACATGCGCCCCGAGATCTGGATCGCCCAAGAAGCACGGCG |
| | GATCGGAGATGAGGCCAACGCCTACTATGCGCGGAGAGGAAGCGGGGTC |
| | AAGCAGACTCTTAACTTCGACCTCCTGAAACTGGCCGGAGATGTGGAGTC |
| | AAACCCTGGACCGGACATGCGGCCGGAAATCTGGATTGCTCAGGAGGCA |
| | CGCAGAATCGGCGATGAAGCCAACGCGTACTACGCCAGACGGGGGAAGC |
| | CTATTCCGAACCCTCTGCTGGGTCTGGACTCCACC |
| | (BimBH3.2A(x3.F2A).v5_miR122) |
| 129 | ATGATCCCTAGGAAGGCCCTGGAGACCCTGAGGAGAGTGGGCGACGGAG |
| | TGCAGAGGAACCACGAGACCGCCTTCGGCCTGAGCGAGGCAAAGCCAGC |
| | AACACCCGAGATCCAGGAGATCGTGGATAAGGTGAAGCCCCAGCTGGAG |
| | GAGAAGACCAATGAGACATACGGCAAGCTGGAGGCCGTGCAGTATAAGA |
| | CCCAGGTGCTGGCCTCCACAAACTACTATATCAAGGTGCGGGCCGGCGAC |
| | AATAAGTACATGCACCTGAAGGTGTTCAACGGACCACCTGGACAGAATGC |
| | AGACAGGGTGCTGACCGGCTATCAGGTGGATAAGAACAAGGACGATGAG |
| | CTGACAGGCTTTGGCAAGCCAATCCCCAATCCTCTGCTGGGCCTGGATTCT |
| | ACA |
| | (SQT.(nt)Mcl1.BH3.cv5) |
| 130 | ATGAAGGCCCTGGAGACCCTGAGGAGAGTGGGCGACGGCGTGCAGAGGA |
| | ACCACGAGACCGCCTTCGGCAGCGGAGTGAAGCAGACACTGAACTTTGAC |
| | CTGCTGAAGCTGGCAGGCGATGTGGAGTCCAATCCAGGACCTAAGGCCCT |
| | GGAGACACTGAGGAGGGTGGGCGATGGAGTGCAGAGGAATCATGAAACC |
| | GCCTTCGGCTCTGGCGTCAAACAGACACTGAACTTCGACCTGCTGAAGCT |
| | GGCCGGCGATGTGGAGAGCAATCCAGGACCAAAGGCCCTGGAGACCCTG |
| | CGCAGAGTGGGCGACGGAGTCCAGAGAAACCACGAGACAGCCTTCGGCA |
| | AGCCTATCCCAAATCCCCTGCTGGGCCTGGATAGCACC |
| | (Mcl1.BH3(x3.F2A).cV5) |
| 131 | ATGAGGCCCGAGATCTGGATGACCCAGGGACTGCGGAGACTGGGCGACG |
| | AGATCAACGCCTACTATGCAAGGGGCAGCGGAGTGAAGCAGACACTGAA |
| | CTTCGACCTGCTGAAGCTGGCAGGCGATGTGGAGTCCAATCCAGGACCTC |
| | GGCCAGAAATTTGGATGACTCAGGGCCTGAGGCGCCTGGGCGATGAGATC |
| | AATGCCTACTATGCCAGAGGCTCTGGCGTCAAACAGACACTGAACTTTGA |
| | CCTGCTGAAGCTGGCCGGCGATGTGGAGAGCAATCCAGGACCTAGGCCA |
| | GAAATCTGGATGACTCAGGGGTTACGGAGACTGGGCGACGAGATCAACG |
| | CGTACTATGCCCGGGGCAAGCCCATCCCTAATCCACTGCTGGGCCTGGAT |
| | AGCACA |
| | (antiMcl1.MS1(x3.F2A).cV5) |
| 132 | ATGCGGCCCGAGATCTGGCTGACCCAGTCCCTGCAGAGACTGGGCGACGA |
| | GATCAACGCCTACTATGCCAGGGGCTCTGGCGTGAAGCAGACACTGAACT |
| | TCGACCTGCTGAAGCTGGCAGGCGATGTGGAGAGCAATCCAGGACCTAG |
| | GCCAGAAATTTGGCTGACTCAGAGCCTGCAGCGGCTGGGCGATGAGATCA |
| | ATGCCTACTATGCCAGAGGCAGCGGAGTCAAGCAGACACTGAACTTTGAC |
| | CTGCTGAAGCTGGCCGGCGATGTGGAGTCTAATCCCGGCCCTAGGCCAGA |
| | AATCTGGCTGACTCAGTCCCTGCAGCGCCTGGGCGACGAGATCAACGCGT |
| | ACTATGCCCGGGGCAAGCCCATCCCTAATCCACTGCTGGGCCTGGATTCC |
| | ACA |
| | (antiMcl1.MS2(x3.F2A).cV5) |
| 133 | ATGCGGCCCGAGATCTGGCTGACCCAGCACCTGCAGAGACTGGGCGACG |
| | AGATCAACGCCTACTATGCCAGGGGCAGCGGCGTGAAGCAGACACTGAA |
| | CTTCGACCTGCTGAAGCTGGCAGGCGATGTGGAGTCCAATCCAGGACCTA |
| | GGCCAGAAATTTGGCTGACTCAGCACCTGCAGCGGCTGGGCGATGAGATC |
| | AATGCCTACTATGCCAGAGGCTCTGGAGTCAAGCAGACACTGAACTTTGA |
| | CCTGCTGAAGCTGGCCGGCGATGTGGAGAGTAACCCAGGACCTAGGCCA |
| | GAGATTTGGTTAACTCAACACCTGCAGCGCCTGGGCGACGAGATCAACGC |
| | GTACTATGCCCGGGGCAAGCCCATCCCTAATCCACTGCTGGGCCTGGATA |
| | GCACA |
| | (antiMcl1.MS3(x3.F2A).cV5) |
| 134 | ATGGACTTCTCCGTGCTGCAGACCATCGGCGATAGCCTGGGCTCCGGCGT |
| | GAAGCAGACACTGAACTTCGACCTGCTGAAGCTGGCAGGCGATGTGGAGT |
| | CCAATCCAGGACCTGACTTTTCTGTGCTGCAGACTATTGGCGATTCTCTGG |
| | GCAGCGGAGTCAAACAGACACTGAACTTTGACCTGCTGAAGCTGGCCGGC |
| | GATGTGGAGAGCAATCCAGGCCCCGACTTTTCCGTGCTGCAAACTATTGG |
| | CGATTCCCTGGGCAAGCCTATCCCAAACCCCCTGCTGGGCCTGGACAGCA |
| | CA |
| | (antiMcl1.SB-02(x3.F2A).cV5) |
| 135 | ATGAACGAGACCGTGAATACAATGCTGACCTACTATTACGGCAGCGGCGT |
| | GAAGCAGACACTGAACTTCGACCTGCTGAAGCTGGCAGGCGATGTGGAG |
| | AGCAACCCAGGACCTAATGAGACAGTGAACACCATGCTGACATATTACTA |
| | TGGCTCCGGCGTGAAGCAGACCCTGAACTTTGACCTGCTGAAGCTGGCCG |
| | GCGATGTGGAGTCTAATCCAGGCCCCAATGAAACTGTCAACACCATGCTG |
| | ACTTATTATTACGGCAAGCCTATCCCAAATCCCCTGCTGGGCCTGGACAG |
| | CACA |
| | (antiMcl1.SB-03(x3.F2A).cV5) |
| 136 | ATGAACGAGACCGTGGAGCTGATGCAGGCCTACCTGCACGGCAGCGGCG |
| | TGAAGCAGACACTGAATTTCGACCTGCTGAAGCTGGCAGGCGATGTGGAG |
| | AGCAACCCAGGACCTAATGAAACTGTCGAACTGATGCAGGCCTATCTGCA |
| | CGGCTCCGGAGTCAAGCAGACACTGAACTTTGACCTGCTGAAGCTGGCCG |
| | GCGATGTGGAGTCTAACCCAGGCCCCAATGAAACTGTCGAGCTGATGCAG |
| | GCTTACCTGCACGGCAAGCCTATCCCAAATCCCCTGCTGGGCCTGGACAG |
| | CACA |
| | (antiMcl1.SB-04(x3.F2A).cV5) |
| 137 | DMRPEIWIAQEARRIGDEANAYYARR |
| | (BimBH3.2A) |
| 138 | GSGVKQTLNFDLLKLAGDVESNPGP |
| | (F2A linker sequence) |
| 139 | GSGEGRGSLLTCGDVEENPGP |
| | (T2A linker sequence) |
| 140 | GSGATNFSLLKQAGDVEENPGP |
| | (P2A linker sequence) |
| 141 | GSGQCTNYALLKLAGDVESNPGP |
| | (E2A linker sequence) |
| 142 | MEEQWAREIGAQLRRMADDLNAQYERR |
| | (PumaBH3_HS3UPCRfree) |
| 143 | MHHHHHHEEQWAREIGAQLRRMADDLNAQYERR |
| | (PumaBH3.nHIS_HS3UPCRfree) |
| 144 | MGKPIPNPLLGLDSTEEQWAREIGAQLRRMADDLNAQYERR |
| | (PumaBH3.nV5_HS3UPCRfree) |
| 145 | MEEQWAREIGAQLRRMADDLNAQYERRHHHHHH |
| | (PumaBH3.cHIS_HS3UPCRfree) |
| 146 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGKPIPNPLLGLDST |
| | (PumaBH3(x3.F2A).v5_Hs3UPCRfree) |
| 147 | MEEQWAREIGAQLRRMADDLNAQYERRGKPIPNPLLGLDST |
| | (PumaBH3.cV5_HS3UPCRfree) |
| 148 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGKPIPNPLLGLDST |
| | (PumaBH3(x3.F2A).v5_miR142.3p_tp) |
| 149 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGKPIPNPLLGLDST |
| | (PumaBH3(x3.F2A).v5_miR122/142.3p_tp) |
| 150 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGKPIPNPLLGLDST |
| | (PumaBH3(x3.F2A).v5_tp) |
| 151 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGKPIPNPLLGLDST |
| | (PumaBH3(x3.F2A).v5_miR122_tp) |
| 152 | MEEQWAREIGAQLRRMADDLNAQYERRGSGVKQTLNFDLLKLAGDVESNP |
| | GPEEQWAREIGAQLRRMADDLNAQYERRGSGVKQTLNFDLKAGDVESNPGP |
| | EEQWAREIGAQLRRMADDNAQYERRGSGVKQTLNFDLLKLAGDVESNPGPE |
| | EQWAREIGAQLRRMADDLNAQYERRGSGVKQTLNFDLLKLAGDVESNPGPE |
| | EQWAREIGAQLRRMADDLNAQYERRGSGVKQTLNFDLLKLAGDVESNPGPG |
| | KPIPNPLLGLDST |
| | ((pumaBH3.F2A)\hat{ }5_cV5, (pumaBH3.F2A)\hat{ }5_cV5_DX) |
| 153 | MDMRPEIWIAQELRRIGDEFNAYYARRGSGVKQTLNFDLLKLAGDVESNPGP |
| | DMRPEIWIAQELRRIGDEFNAYYARRGSGVKQTLNFDLLKLAGDVESNPGPD |
| | MRPEIWIAQELRRIGDEFNAYYARRGSGVKQTLNFDLLKLAGDVESNPGPDM |
| | RPEIWIAQELRRIGDEFNAYYARRGSGVKQTLNFDLLKLAGDVESNPGPDMR |
| | PEIWIAQELRRIGDEFNAYYARRGSGVKQTLNFDLLKLAGDVESNPGPGKPIP |
| | NPLLGLDST |
| | ((BimBH3.F2A)\hat{ }5_cV5, (BimBH3.F2A)\hat{ }5_cV5_DX) |
| 154 | MQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PGSGVKQTLNFDLLKLAGDVESNPGPGKPIPNPLLGLDST |
| | ((SuperPumaBH3.F2A)\hat{ }3_cV5) |
| 155 | MQWAREIGAQLRRIGDDLNAQYERRRQGSVKQTLNFDLLKLAGDVESNPGP |
| | QWAREIGAQRRIGDDLNAQYERRRQGSGVKQTLNFDLLKLAGDVESNPGPQ |
| | WAREIGAQLRRIGDDLNAQYERRRQGSGVKQTLNFDLLKLAGDVESNPGPQ |
| | AREIGAQLRRIGDDLNAQYERRRQGSGVKQTNFDLLKLAGDVESNPGPQWA |
| | REIGAQLRRIGDLNAQYERRRQGSGVKQTLNFDLLKLAGDVENPGPGKPIPNP |
| | LLGLDST ((SuperPumaBH3.F2A)\hat{ }5_cV5) |
| 156 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESN |
| | PGPQWAREIGAQERREADDENAQYERRRQGSGVKQTLNFDLLKLAGDVES |
| | NPGPQWAREIGAQERREADDENAQYERRRQGSGVKQTLNFDLLKLAGDVE |
| | SNPGPQWAREIGAQERREADDENAQYERRRQGSGVKQTLNFDLLKLAGDV |
| | ESNPGPGKPIPNPLLGLDST |
| | ((KittyCatBH3.F2A)\hat{ }5_cV5,(KittyCatBH3.F2A)\hat{ }5_cV5_DX) |
| 157 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESN |
| | PGPQWAREIGAQERREADDENAQYERRRQGSGVKQTLNFDLLKLAGDVES |
| | NPGPQWAREIGAQERREADDENAQYERRRQGSGVKQTLNFDLLKLAGDVE |
| | SNPGPQWAREIGAQERREADDENAQYERRRQGSGVKQTLNFDLLKLAGDV |
| | ESNPGPGKPIPNPLLGLDST |
| | ((KittyCatBH3.F2A)\hat{ }5_cV5, (KittyCatBH3.F2A)\hat{ }5_cV5_DX) |
| 158 | MNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPGKPIPNPLLGLDST |
| | ((BadBH3.F2A)\hat{ }5_cV5) |
| 159 | MPAELEVECATQLRRFGDKLNFRQKLLGSGVKQTLNFDLLKLAGDVESNPGP |
| | NLWAAQRYGRELRRMSDEFVDSFKKGGSGVKQTLNFDLLKLAGDVESNPGP |
| | PAELEVECATQLRRFGDKLNFRQKLLGSGVKQTLNFDLLKLAGDVESNPGPN |
| | LWAAQRYGRELRRMSDEFVDSFKKGGSGVKQTLNFDLLKLAGDVESNPGPP |
| | AELEVECATQLRRFGDKLNFRQKLLGSGVKQTLNFDLLKLAGDVESNPGPNL |
| | WAAQRYGRELRRMSDEFVDSFKKGGSGVKQTLNFDLLKLAGDVESNPGPGK |
| | PIPNPLLGLDST |
| | ((Noxa-F2A-BadBH3-F2A)\hat{ }3_cV5) |
| 160 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPGKPIPNPLLGLDST |
| | ((pumaBH3.F2A)\hat{ }10_cV5, (pumaBH3.F2A)\hat{ }10_cV5_DX) |
| 161 | MDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PGKPIPNPLLGLDST |
| | ((BimBH3.F2A)\hat{ }10_cV5, (BimBH3.F2A)\hat{ }10_cV5_DX) |
| 162 | MEEQWAREIGAQLRRMADDLNAQYERRGSGVKQTLNFDLLKLAGDVESNP |
| | GPDMRPEIWIAQELRRIGDEFNAYYARRGSGVKQTLNFDLLKLAGDVESNPG |
| | PEEQWAREIGAQLRRMADDLNAQYERRGSGVKQTLNFDLLKLAGDVESNPG |
| | PDMRPEIWIAQELRRIGDEFNAYYARRGSGVKQTLNFDLLKLAGDVESNPGP |
| | EEQWAREIGAQLRRMADDLNAQYERRGSGVKQTLNFDLLKLAGDVESNPGP |
| | DMRPEIWIAQELRRIGDEFNAYYARRGSGVKQTLNFDLLKLAGDVESNPGPG |
| | KPIPNPLLGLDST |
| | ((Puma-P2A-BimBH3-F2A)\hat{ }3_cV5) |
| 163 | MEEQWAREIGA QLRRMADDLNAQYERRGSGVKQTLNFDLLKLAGDVESNP |
| | GPDMRPEIWIAQELRRIGDEFNAYYARRGSGVKQTLNFDLLKLAGDVESNPG |
| | PEEQWAREIGAQLRRMADDLNAQYERRGSGVKQTLNFDLLKLAGDVESNPG |
| | PDMRPEIWIAQELRRIGDEFNAYYARRGSGVKQTLNFDLLKLAGDVESNPGP |
| | EEQWAREIGAQLRRMADDLNAQYERRGSGVKQTLNFDLLKLAGDVESNPGP |
| | DMRPEIWIAQELRRIGDEFNAYYARRGSGVKQTLNFDLLKLAGDVESNPGPE |
| | EQWAREIGAQLRRMADDLNAQYERRGSGVKQTLNFDLLKLAGDVESNPGPD |
| | MRPEIWIAQELRRIGDEFNAYYARRGSGVKQTLNFDLLKLAGDVESNPGPEE |
| | QWAREIGAQLRRMADDLNAQYERRGSGVKQTLNFDLLKLAGDVESNPGPD |
| | MRPEIWIAQELRRIGDEFNAYYARRGSGVKQTLNFDLLKLAGDVESNPGPGK |
| | PIPNPLLGLDST |
| | ((Puma-F2A-BimBH3-F2A)\hat{ }5_cV5, (Puma-F2A-BimBH3F2A)\hat{ }5_cV5_DX) |
| 164 | MEEQWAREIGAQLRRMADDLNAQYERRGSGVKQTLNFDLLKLAGDVESN |
| | PGPDMRPEIWIAQELRRIGDEFNAYYARRGSGVKQTLNFDLLKLAGDVESN |
| | PGPEEQWAREIGAQLRRMADDLNAQYERRGSGVKQTLNFDLLKLAGDVES |
| | NPGPDMRPEIWIAQELRRIGDEFNAYYARRGSGVKQTLNFDLLKLAGDVEN |
| | PGPEEQWAREIGAQLRRMADDLNAQYERRGSGVKQTLNFDLLKLAGDVES |
| | NPGPDMRPEIWIAQERRIGDEFNAYYARRGSGVKQTLNFDLLKLAGDVESN |
| | PGPEEQWAREIGAQLRRMADDLNAQYERRGSGVKQTLNFDLLKLAGDVES |
| | NPGPDMRPEIWIAQELRRIGDEFNAYYARRGSGVKQTLNFDLLKLAGDVES |
| | NPGPEEQWAREIGAQLRRMADDLNAQYERRGSGVKQTLNFDLLKLAGDVE |
| | SNPGPDMRPEIWIAQELRRIGDEFNAYYARRGSGVKQTLNFDLLKLAGDVE |
| | SNPGPEEQWAREIGAQLRRMADDLNAQYERRGSGVKQTLNFDLLKLAGDV |
| | ESNPGPDMRPEIWIAQELRRIGDEFNAYYARRGSGVKQTLNFDLLKLAGDV |
| | ESNPGPEEQWAREIGAQLRRMADDLNAQYERRGSGVKQTLNFDLLKLAGD |
| | VESNPGPDMRPEIWIAQELRRIGDEFNAYYARRGSGVKQTLNFDLLKLAGD |
| | VESNPGPEEQWAREIGAQLRRMADDLNAQYERRGSGVKQTLNFDLLKLAG |
| | DVESNPGPDMRPEIWIAQELRRIGDEFNAYYARRGSGVKQTLNFDLLKLAG |
| | DVESNPGPEEQWAREIGAQLRRMADDLNAQYERRGSGVKQTLNFDLLKLA |
| | GDVESNPGPDMRPEIWIAQELRRIGDEFNAYYARRGSGVKQTLNFDLLKLA |
| | GDVESNPGPEEQWAREIGAQLRRMADDLNAQYERRGSGVKQTLNFDLLKL |
| | AGDVESNPGPDMRPEIWIAQELRRIGDEFNAYYARRGSGVKQTLNFDLLKL |
| | AGDVESNPGPGKPIPNPLLGLDST |
| | ((Puma-F2A-BimBH3-F2A)\hat{ }10_cV5) |
| 165 | MQWAREIGAQLRRIGDDLNAQYERRRQGSGVKQTLNFDLLKLAGDVESNP |
| | GPQWAREIGAQLRRIGDDLNAQYERRRQGSGVKQTLNFDLLKLAGDVESN |
| | PGPQWAREIGAQLRRIGDDLNAQYERRRQGSGVKQTLNFDLLKLAGDVES |
| | NPGPQWAREIGAQLRRIGDDLNAQYERRRQGSGVKQTLNFDLLKLAGDES |
| | NPGPQWAREIGAQLRRIGDDLNAQYERRRQGSGVKQTLNFDLLKLAGDVE |
| | SNPGPQWAREIGAQLRRIGDDLNAQYERRRQGSGVKQTLNFDLLKLAGDV |
| | ESNPGPQWAREIGAQLRRIGDDLNAQYERRRQGSGVKQTLNFDLLKLAGD |
| | VESNPGPQWAREIGAQLRRIGDDLNAQYERRRQGSGVKQTLNFDLLKLAG |
| | DVESNPGPQWAREIGAQLRRIGDDLNAQYERRRQGSGVKQTLNFDLLKLA |
| | GDVESNPGPQWAREIGAQLRRIGDDLNAQYERRRQGSGVKQTLNFDLLKL |
| | AGDVESNPGPGKPIPNPLLGLDST |
| | ((SuperPumaBH3.F2A)\hat{ }10_cV5, (SuperPumaBH3.F2A)\hat{ }10_cV5_DX) |
| 166 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESN |
| | PGPQWAREIGAQERREADDENAQYERRRQGSGVKQTLNFDLLKLAGDVES |
| | NPGPQWAREIGAQERREADDENAQYERRRQGSGVKQTLNFDLLKLAGDVE |
| | SNPGPQWAREIGAQERREADDENAQYERRRQGSGVKQTLNFDLLKLAGDV |
| | ESNPGPQWAREIGAQERREADDENAQYERRRQGSGVKQTLNFDLLKLAGD |
| | VESNPGPQWAREIGAQERREADDENAQYERRRQGSGVKQTLNFDLLKLAG |
| | DVESNPGPQWAREIGAQERREADDENAQYERRRQGSGVKQTLNFDLLKLA |
| | GDVESNPGPQWAREIGAQERREADDENAQYERRRQGSGVKQTLNFDLLKL |
| | AGDVESNPGPQWAREIGAQERREADDENAQYERRRQGSGVKQTLNFDLLK |
| | LAGDVESNPGPQWAREIGAQERREADDENAQYERRRQGSGVKQTLNFDLL |
| | KLAGDVESNPGPGKPIPNPLLGLDST |
| | ((KittyCatBH3.F2A)\hat{ }10_cV5, (KittyCatBH3.F2A)\hat{ }10_cV5_DX) |
| 167 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESN |
| | PGPQWAREIGAQERREADDENAQYERRRQGSGVKQTLNFDLLKLAGDVES |
| | NPGPQWAREIGAQERREADDENAQYERRRQGSGVKQTLNFDLLKLAGDVE |
| | SNPGPQWAREIGAQERREADDENAQYERRRQGSGVKQTLNFDLLKLAGDV |
| | ESNPGPQWAREIGAQERREADDENAQYERRRQGSGVKQTLNFDLLKLAGD |
| | VESNPGPQWAREIGAQERREADDNAQYERRRQGSGVKQTLNFDLLKLAGD |
| | VESNPGPQWAREIGAQERREADDENAQYERRRQGSGVKQTLNFDLLKLAG |
| | DVESNPGPQWAREIGAQERREADDENAQYERRRQGSGVKQTLNFDLLKLA |
| | GDVESNPGPQWAREIGAQERREADDENAQYERRRQGSGVKQTLNFDLLKL |
| | AGDVESNPGPQWAREIGAQERREADDENAQYERRRQGSGVKQTLNFDLLK |
| | LAGDVESNPGPGKPIPNPLLGLDST |
| | ((KittyCatBH3.F2A)\hat{ }10_cV5, (KittyCatBH3.F2A)\hat{ }10_cV5_DX) |
| 168 | MPAELEVECATQLRRFGDKLNFRQKLLGSGVKQTLNFDLLKLAGDVESNPGP |
| | PAELEVECATQLRRFGDKLNFRQKLLGSGVKQTLNFDLLKLAGDVESNPGPP |
| | AELEVECATQLRRFGDKLNFRQKLLGSGVKQTLNFDLLKLAGDVESNPGPPA |
| | ELEVECATQLRRFGDKLNFRQKLLGSGVKQTLNFDLLKLAGDVESNPGPPAE |
| | LEVECATQLRRFGDKLNFRQKLLGSGVKQTLNFDLLKLAGDVESNPGPPAEL |
| | EVECATQLRRFGDKLNFRQKLLGSGVKQTLNFDLLKLAGDVESNPGPGKPIP |
| | NPLLGLDST |
| | ((NoxaBH3.F2A)\hat{ }5_cV5, (NoxaBH3.F2A)\hat{ }5_cV5_DX) |
| 169 | MPAELEVECATQLRRFGDKLNFRQKLLGSGVKQTLNFDLLKLAGDVESNPGP |
| | PAELEVECATQLRRFGDKLNFRQKLLGSGVKQTLNFDLLKLAGDVESNPGPP |
| | AELEVECATQLRRFGDKLNFRQKLLGSGVKQTLNFDLLKLAGDVESNPGPPA |
| | ELEVECATQLRRFGDKLNFRQKLLGSGVKQTLNFDLLKLAGDVESNPGPPAE |
| | LEVECATQLRRFGDKLNFRQKLLGSGVKQTLNFDLLKLAGDVESNPGPPAEL |
| | EVECATQLRRFGDKLNFRQKLLGSGVKQTLNFDLLKLAGDVESNPGPPAELE |
| | VECATQLRRFGDKLNFRQKLLGSGVKQTLNFDLLKLAGDVESNPGPPAELEV |
| | ECATQLRRFGDKLNFRQKLLGSGVKQTLNFDLLKLAGDVESNPGPPAELEVE |
| | CATQLRRFGDKLNFRQKLLGSGVKQTLNFDLLKLAGDVESNPGPPAELEVEC |
| | ATQLRRFGDKLNFRQKLLGSGVKQTLNFDLLKLAGDVESNPGPPAELEVECA |
| | TQLRRFGDKLNFRQKLLGSGVKQTLNFDLLKLAGDVESNPGPGKPIPNPLLGL |
| | DST |
| | ((NoxaBH3.F2A)\hat{ }10_cV5, (NoxaBH3.F2A)\hat{ }10_cV5_DX) |
| 170 | MNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPGKPIPNPLLGLDST |
| | ((BadBH3.F2A)\hat{ }10_cV5,(BadBH3.F2A)\hat{ }10_cV5_DX) |
| 171 | MPAELEVECATQLRRFGDKLNFRQKLLGSGVKQTLNFDLLKLAGDVESNPGP |
| | NLWAAQRYGRELRRMSDEFVDSFKKGGSGVKQTLNFDLLKLAGDVESNPGP |
| | PAELEVECATQLRRFGDKLNFRQKLLGSGVKQTLNFDLLKLAGDVESNPGPN |
| | LWAAQRYGRELRRMSDEFVDSFKKGGSGVKQTLNFDLLKLAGDVESNPGPP |
| | AELEVECATQLRRFGDKLNFRQKLLGSGVKQTLNFDLLKLAGDVESNPGPNL |
| | WAAQRYGRELRRMSDEFVDSFKKGGSGVKQTLNFDLLKLAGDVESNPGPPA |
| | ELEVECATQLRRFGDKLNFRQKLLGSGVKQTLNFDLLKLAGDVESNPGPNL |
| | WAAQRYGRELRRMSDEFVDSFKKGGSGVKQTLNFDLLKLAGDVESNPGPPA |
| | ELEVECATQLRRFGDKLNFRQKLLGSGVKQTLNFDLLKLAGDVESNPGPNL |
| | WAAQRYGRELRRMSDEFVDSFKKGGSGVKQTLNFDLLKLAGDVESNPGPGK |
| | PIPNPLLGLDST |
| | ((Noxa-F2A-BadBH3-F2A)\hat{ }5_cV5, (Noxa-F2A-BadBH3-F2A)\hat{ }5_cV5_DX) |
| 172 | MPAELEVECATQLRRFGDKLNFRQKLLGSGVKQTLNFDLLKLAGDVESNPG |
| | PNLWAAQRYGRELRRMSDEFVDSFKKGGSGVKQTLNFDLLKLAGDVESNP |
| | GPPAELEVECATQLRRFGDKLNFRQKLLGSGVKQTLNFDLLKLAGDVESNP |
| | GPNLWAAQRYGRELRRMSDEFVDSFKKGGSGVKQTLNFDLLKLAGDVESN |
| | GPPAELEVECATQLRRFGDKLNFRQKLLGSGVKQTLNFDLLKLAGDVESNP |
| | GPNLWAAQRYGRELRRMSDEFVDSFKKGGSGVKQTLNFDLLKLAGDVESN |
| | PGPPAELEVECATQLRRFGDKLNFRQKLLGSGVKQTLNFDLLKLAGDVESN |
| | PGPNLWAAQRYGRELRRMSDEFVDSFKKGGSGVKQTLNFDLLKLAGDVES |
| | NPGPPAELEVECATQLRRFGDKLNFRQKLLGSGVKQTLNFDLLKLAGDVES |
| | NPGPNLWAAQRYGRELRRMSDEFVDSFKKGGSGVKQTLNFDLLKLAGDVE |
| | SNPGPPAELEVECATQLRRFGDKLNFRQKLLGSGVKQTLNFDLLKLAGDVE |
| | SNPGPNLWAAQRYGRELRRMSDEFVDSFKKGGSGVKQTLNFDLLKLAGDV |
| | ESNPGPPAELEVECATQLRRFGDKLNFRQKLLGSGVKQTLNFDLLKLAGDV |
| | ESNPGPNLWAAQRYGRELRRMSDEFVDSFKKGGSGVKQTLNFDLLKLAGD |
| | VESNPGPPAELEVECATQLRRFGDKLNFRQKLLGSGVKQTLNFDLLKLAGD |
| | VESNPGPNLWAAQRYGRELRRMSDEFVDSFKKGGSGVKQTLNFDLLKLAG |
| | DVESNPGPPAELEVECATQLRRFGDKLNFRQKLLGSGVKQTLNFDLLKLAG |
| | DVESNPGPNLWAAQRYGRELRRMSDEFVDSFKKGGSGVKQTLNFDLLKLA |
| | GDVESNPGPPAELEVECATQLRRFGDKLNFRQKLLGSGVKQTLNFDLLKLA |
| | GDVESNPGPNLWAAQRYGRELRRMSDEFVDSFKKGGSGVKQTLNFDLLKL |
| | AGDVESNPGPGKPIPNPLLGLDST |
| | ((Noxa-F2A-BadBH3-F2A)\hat{ }10_cV5) |
| 173 | MEEQWAREIGAQLRRMADDLNAQYERRGSGVKQTLNFDLLKLAGDVESNP |
| | GPDMRPEIWIAQELRRIGDEFNAYYARRGSGVKQTLNFDLLKLAGDVESNPG |
| | PEEQWAREIGAQLRRMADDLNAQYERRGSGVKQTLNFDLLKLAGDVESNPG |
| | PDMRPEIWIAQELRRIGDEFNAYYARRGSGVKQTLNFDLLKLAGDVESNPGP |
| | EEQWAREIGAQLRRMADDLNAQYERRGSGVKQTLNFDLLKLAGDVESNPGP |
| | DMRPEIWIAQELRRIGDEFNAYYARRGSGVKQTLNFDLLKLAGDVESNPGPG |
| | KPIPNPLLGLDST |
| | ((Puma-F2A-BimBH3-F2A)\hat{ }3_cV5_DX) |
| 174 | MQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PGSGVKQTLNFDLLKLAGDVESNPGPGKPIPNPLLGLDST |
| | ((SuperPumaBH3.F2A)\hat{ }3_cV5_DNA2.0) |
| 175 | MEEQWAREIGAQLRRMADDLNAQYERRGSGVKQTLNFDLLKLAGDVESNP |
| | GPDMRPEIWIAQELRRIGDEFNAYYARRGSGVKQTLNFDLLKLAGDVESNPG |
| | PEEQWAREIGAQLRRMADDLNAQYERRGSGVKQTLNFDLLKLAGDVESNPG |
| | PDMRPEIWIAQELRRIGDEFNAYYARRGSGVKQTLNFDLLKLAGDVESNPGP |
| | EEQWAREIGAQLRRMADDLNAQYERRGSGVKQTLNFDLLKLAGDVESNPGP |
| | DMRPEIWIAQELRRIGDEFNAYYARRGSGVKQTLNFDLLKLAGDVESNPGPG |
| | KPIPNPLLGLDST |
| | ((Puma-F2A-BimBH3-F2A)\hat{ }3_cV5_DX_DNA2.0) |
| 176 | MQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PGKPIPNPLLGLDST |
| | ((SuperPumaBH3.F2A)\hat{ }5_cV5_DNA2.0) |
| 177 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPGKPIPNPLLGLDST |
| | ((pumaBH3.F2A)\hat{ }5_cV5_DNA2.0) |
| 178 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESN |
| | PGPQWAREIGAQERREADDENAQYERRRQGSGVKQTLNFDLLKLAGDVES |
| | NPGPQWAREIGAQERREADDENAQYERRRQGSGVKQTLNFDLLKLAGDVE |
| | SNPGPQWAREIGAQERREADDENAQYERRRQGSGVKQTLNFDLLKLAGDV |
| | ESNPGPGKPIPNPLLGLDST |
| | ((KittyCatBH3.F2A)\hat{ }5_cV5_DNA2.0) |
| 179 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESN |
| | PGPQWAREIGAQERREADDENAQYERRRQGSGVKQTLNFDLLKLAGDVES |
| | NPGPQWAREIGAQERREADDENAQYERRRQGSGVKQTLNFDLLKLAGDVE |
| | SNPGPQWAREIGAQERREADDENAQYERRRQGSGVKQTLNFDLLKLAGDV |
| | ESNPGPQWAREIGAQERREADDENAQYERRRQGSGVKQTLNFDLLKLAGD |
| | VESNPGPQWAREIGAQERREADDENAQYERRRQGSGVKQTLNFDLLKLAG |
| | DVESNPGPQWAREIGAQERREADDENAQYERRRQGSGVKQTLNFDLLKLA |
| | GDVESNPGPQWAREIGAQERREADDENAQYERRRQGSGVKQTLNFDLLKL |
| | AGDVESNPGPQWAREIGAQERREADDENAQYERRRQGSGVKQTLNFDLLK |
| | LAGDVESNPGPQWAREIGAQERREADDENAQYERRRQGSGVKQTLNFDLL |
| | KLAGDVESNPGPGKPIPNPLLGLDST |
| | (KittyCatBH3.F2A)\hat{ }10_cV5_DNA2.0) |
| 180 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPGKPIPNPLLGLDST |
| | ((pumaBH3.F2A)\hat{ }10_cV5_DNA2.0) |
| 181 | MEEQWAREIGAQLRRMADDLNAQYERRGSGVKQTLNFDLLKLAGDVESNP |
| | GPDMRPEIWIAQELRRIGDEFNAYYARRGSGVKQTLNFDLLKLAGDVESNPG |
| | PEEQWAREIGAQLRRMADDLNAQYERRGSGVKQTLNFDLLKLAGDVESNPG |
| | PDMRPEIWIAQELRRIGDEFNAYYARRGSGVKQTLNFDLLKLAGDVESNPGP |
| | EEQWAREIGAQLRRMADDLNAQYERRGSGVKQTLNFDLLKLAGDVESNPGP |
| | DMRPEIWIAQELRRIGDEFNAYYARRGSGVKQTLNFDLLKLAGDVESNPGPE |
| | EQWAREIGAQLRRMADDLNAQYERRGSGVKQTLNFDLLKLAGDVESNPGPD |
| | MRPEIWIAQELRRIGDEFNAYYARRGSGVKQTLNFDLLKLAGDVESNPGPEE |
| | QWAREIGAQLRRMADDLNAQYERRGSGVKQTLNFDLLKLAGDVESNPGPD |
| | MRPEIWIAQELRRIGDEFNAYYARRGSGVKQTLNFDLLKLAGDVESNPGPGK |
| | PIPNPLLGLDST |
| | ((Puma-F2A-BimBH3-F2A)\hat{ }5_cV5_DNA2.0) |
| 182 | MQWAREIGAQLRRIGDDLNAQYERRRQGSGVKQTLNFDLLKLAGDVESNP |
| | GPQWAREIGAQLRRIGDDLNAQYERRRQGSGVKQTLNFDLLKLAGDVESN |
| | PGPQWAREIGAQLRRIGDDLNAQYERRRQGSGVKQTLNFDLLKLAGDVES |
| | NPGPQWAREIGAQLRRIGDDLNAQYERRRQGSGVKQTLNFDLLKLAGDVE |
| | SNPGPQWAREIGAQLRRIGDDLNAQYERRRQGSGVKQTLNFDLLKLAGDV |
| | ESNPGPQWAREIGAQLRRIGDDLNAQYERRRQGSGVKQTLNFDLLKLAGD |
| | VESNPGPQWAREIGAQLRRIGDDLNAQYERRRQGSGVKQTLNFDLLKLAG |
| | DVESNPGPQWAREIGAQLRRIGDDLNAQYERRRQGSGVKQTLNFDLLKLA |
| | GDVESNPGPQWAREIGAQLRRIGDDLNAQYERRRQGSGVKQTLNFDLLKL |
| | AGDVESNPGPQWAREIGAQLRRIGDDLNAQYERRRQGSGVKQTLNFDLLK |
| | LAGDVESNPGPGKPIPNPLLGLDST |
| | ((SuperPumaBH3.F2A)\hat{ }10_cV5_DNA2.0) |
| 183 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESNP |
| | GPQWAREIGAQERREADDENAQYERRRQGSGVKQTLNFDLLKLAGDVESNP |
| | GPGKPIPNPLLGLDST |
| | ((KittyCatBH3.F2A)\hat{ }3_cV5) |
| 184 | MQWAREIGAQERREADDENAQYERRRQGSGATNFSLLKQAGDVEENPGPQ |
| | WAREIGAQERREADDENAQYERRRQGSGATNFSLLKQAGDVEENPGPQWA |
| | REIGAQERREADDENAQYERRRQGSGVKQTLNFDLLKLAGDVESNPGPGKPI |
| | PNPLLGLDST |
| | ((KittyCatBH3.P2A)\hat{ }3_cV5) |
| 185 | MEEQWAREIGAQLRRMADDLNAQYERRGSGATNFSLLKQAGDVEENPGPEE |
| | QWAREIGAQLRRMADDLNAQYERRGSGATNFSLLKQAGDVEENPGPEEQW |
| | AREIGAQLRRMADDLNAQYERRGKPIPNPLLGLDST |
| | (PumaBH3(x3.P2A).v5_miR122) |
| 186 | MEEQWAREIGAQLRRMADDLNAQYERRGGGSGGGSGGGSEEQWAREIGAQ |
| | LRRMADDLNAQYERRGGGSGGGSGGGSEEQWAREIGAQLRRMADDLNAQY |
| | ERRGKPIPNPLLGLDST |
| | ((PUMA BH3.GGGSx3)x3.V5) |
| 187 | MEEQWAREIGAQLRRMADDLNAQYERRGGGSGGGSGGGSEEQWAREIGAQ |
| | LRRMADDLNAQYERRGGGSGGGSGGGSEEQWAREIGAQLRRMADDLNAQY |
| | ERRGKPIPNPLLGLDST |
| | ((PUMA BH3.GGGSx3)x3.V5_DX) |
| 188 | MEEQWAREIGAQLRRMADDLNAQYERRGGGSGGGSGGGSEEQWAREIGAQ |
| | LRRMADDLNAQYERRGKPIPNPLLGLDST |
| | (PUMA BH3.GGGSx3.PUMA BH3.V5) |
| 189 | MEEQWAREIGAQLRRMADDLNAQYERRGSGATNFSLLKQAGDVEENPGPEE |
| | QWAREIGAQLRRMADDLNAQYERRGSGATNFSLLKQAGDVEENPGPEEQW |
| | AREIGAQLRRMADDLNAQYERRGKPIPNPLLGLDST |
| | (PumaBH3(x3.P2A).v5_miR122) |
| 190 | MEEQWAREIGAQLRRMADDLNAQYERR |
| | (PumaBH3_HS3UPCRfree) |
| 191 | MEEQWAREIGAQLRRMADDLNAQYERR |
| | (PumaBH3_HS3UPCRfree without His Tag) |
| 192 | MEEQWAREIGAQLRRMADDLNAQYERR |
| | (PumaBH3_HS3UPCRfree without v5 tag) |
| 193 | MEEQWAREIGAQLRRMADDLNAQYERR |
| | (PumaBH3_HS3UPCRfree without His tag) |
| 194 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERR |
| | (PumaBH3(x3.F2A)_Hs3UPCRfree without v5 tag) |
| 195 | MEEQWAREIGAQLRRMADDLNAQYERR |
| | (PumaBH3_HS3UPCRfree without the v5 tag) |
| 196 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERR |
| | (PumaBH3(x3.F2A)_miR142.3p_tp without the v5 tag) |
| 197 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERR |
| | (PumaBH3(x3.F2A)_miR122/142.3p_tp without the v5 tag) |
| 198 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERR |
| | (PumaBH3(x3.F2A) tp without the v5 tag) |
| 199 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERR |
| | (PumaBH3(x3.F2A)_miR122_tp without the v5 tag) |
| 200 | MEEQWAREIGAQLRRMADDLNAQYERRGSGVKQTLNFDLLKLAGDVESNP |
| | GPEEQWAREIGAQLRRMADDLNAQYERRGSGVKQTLNFDLKAGDVESNPGP |
| | EEQWAREIGAQLRRMADDNAQYERRGSGVKQTLNFDLLKLAGDVESNPGPE |
| | EQWAREIGAQLRRMADDLNAQYERRGSGVKQTLNFDLLKLAGDVESNPGPE |
| | EQWAREIGAQLRRMADDLNAQYERRGSGVKQTLNFDLLKLAGDVESNPGP |
| | ((pumaBH3.F2A)\hat{ }5, (pumaBH3.F2A)\hat{ }5_DX without the v5 tag) |
| 201 | MDMRPEIWIAQELRRIGDEFNAYYARRGSGVKQTLNFDLLKLAGDVESNPGP |
| | DMRPEIWIAQELRRIGDEFNAYYARRGSGVKQTLNFDLLKLAGDVESNPGPD |
| | MRPEIWIAQELRRIGDEFNAYYARRGSGVKQTLNFDLLKLAGDVESNPGPDM |
| | RPEIWIAQELRRIGDEFNAYYARRGSGVKQTLNFDLLKLAGDVESNPGPDMR |
| | PEIWIAQELRRIGDEFNAYYARRGSGVKQTLNFDLLKLAGDVESNPGP |
| | ((BimBH3.F2A)\hat{ }5, (BimBH3.F2A)\hat{ }5_DX without the v5 tag) |
| 202 | MQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PGSGVKQTLNFDLLKLAGDVESNPGP |
| | ((SuperPumaBH3.F2A)\hat{ }3 without the v5 tag) |
| 203 | MQWAREIGAQLRRIGDDLNAQYERRRQGSVKQTLNFDLLKLAGDVESNPGP |
| | QWAREIGAQRRIGDDLNAQYERRRQGSGVKQTLNFDLLKLAGDVESNPGPQ |
| | WAREIGAQLRRIGDDLNAQYERRRQGSGVKQTLNFDLLKLAGDVESNPGPQ |
| | AREIGAQLRRIGDDLNAQYERRRQGSGVKQTNFDLLKLAGDVESNPGPQWA |
| | REIGAQLRRIGDLNAQYERRRQGSGVKQTLNFDLLKLAGDVENPGP |
| | ((SuperPumaBH3.F2A)\hat{ }5 without the v5 tag) |
| 204 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESN |
| | PGPQWAREIGAQERREADDENAQYERRRQGSGVKQTLNFDLLKLAGDVES |
| | NPGPQWAREIGAQERREADDENAQYERRRQGSGVKQTLNFDLLKLAGDVE |
| | SNPGPQWAREIGAQERREADDENAQYERRRQGSGVKQTLNFDLLKLAGDV |
| | ESNPGP |
| | ((KittyCatBH3.F2A)\hat{ }5,(KittyCatBH3.F2A)\hat{ }5_DX without the v5 tag) |
| 205 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESN |
| | PGPQWAREIGAQERREADDENAQYERRRQGSGVKQTLNFDLLKLAGDVES |
| | NPGPQWAREIGAQERREADDENAQYERRRQGSGVKQTLNFDLLKLAGDVE |
| | SNPGPQWAREIGAQERREADDENAQYERRRQGSGVKQTLNFDLLKLAGDV |
| | ESNPGPGKPIPNPLLGLDST |
| | ((KittyCatBH3.F2A)\hat{ }5, (KittyCatBH3.F2A)\hat{ }5_DX without the v5 tag) |
| 206 | MNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GP |
| | ((BadBH3.F2A)\hat{ }5 without the v5 tag) |
| 207 | MPAELEVECATQLRRFGDKLNFRQKLLGSGVKQTLNFDLLKLAGDVESNPGP |
| | NLWAAQRYGRELRRMSDEFVDSFKKGGSGVKQTLNFDLLKLAGDVESNPGP |
| | PAELEVECATQLRRFGDKLNFRQKLLGSGVKQTLNFDLLKLAGDVESNPGPN |
| | LWAAQRYGRELRRMSDEFVDSFKKGGSGVKQTLNFDLLKLAGDVESNPGPP |
| | AELEVECATQLRRFGDKLNFRQKLLGSGVKQTLNFDLLKLAGDVESNPGPNL |
| | WAAQRYGRELRRMSDEFVDSFKKGGSGVKQTLNFDLLKLAGDVESNPGP |
| | ((Noxa-F2A-BadBH3-F2A)\hat{ }3 without the v5 tag) |
| 208 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GP |
| | ((pumaBH3.F2A)\hat{ }10, (pumaBH3.F2A)\hat{ }10_DX without the v5 tag) |
| 209 | MDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | PDMRPEIWIAQELRRIGDEFNAYYARRGSGVKQTLNFDLLKLAGDVESNPG |
| | P |
| | ((BimBH3.F2A)\hat{ }10, (BimBH3.F2A)\hat{ }10_DX without the v5 tag) |
| 210 | MEEQWAREIGAQLRRMADDLNAQYERRGSGVKQTLNFDLLKLAGDVESNP |
| | GPDMRPEIWIAQELRRIGDEFNAYYARRGSGVKQTLNFDLLKLAGDVESNPG |
| | PEEQWAREIGAQLRRMADDLNAQYERRGSGVKQTLNFDLLKLAGDVESNPG |
| | PDMRPEIWIAQELRRIGDEFNAYYARRGSGVKQTLNFDLLKLAGDVESNPGP |
| | EEQWAREIGAQLRRMADDLNAQYERRGSGVKQTLNFDLLKLAGDVESNPGP |
| | DMRPEIWIAQELRRIGDEFNAYYARRGSGVKQTLNFDLLKLAGDVESNPGP |
| | ((Puma-P2A-BimBH3-F2A)\hat{ }3 without the v5 tag) |
| 211 | MEEQWAREIGAQLRRMADDLNAQYERRGSGVKQTLNFDLLKLAGDVESNP |
| | GPDMRPEIWIAQELRRIGDEFNAYYARRGSGVKQTLNFDLLKLAGDVESNPG |
| | PEEQWAREIGAQLRRMADDLNAQYERRGSGVKQTLNFDLLKLAGDVESNPG |
| | PDMRPEIWIAQELRRIGDEFNAYYARRGSGVKQTLNFDLLKLAGDVESNPGP |
| | EEQWAREIGAQLRRMADDLNAQYERRGSGVKQTLNFDLLKLAGDVESNPGP |
| | DMRPEIWIAQELRRIGDEFNAYYARRGSGVKQTLNFDLLKLAGDVESNPGPE |
| | EQWAREIGAQLRRMADDLNAQYERRGSGVKQTLNFDLLKLAGDVESNPGPD |
| | MRPEIWIAQELRRIGDEFNAYYARRGSGVKQTLNFDLLKLAGDVESNPGPEE |
| | QWAREIGAQLRRMADDLNAQYERRGSGVKQTLNFDLLKLAGDVESNPGPD |
| | MRPEIWIAQELRRIGDEFNAYYARRGSGVKQTLNFDLLKLAGDVESNPGP |
| | ((Puma-F2A-BimBH3-F2A)\hat{ }5, (Puma-F2A-BimBH3F2A)\hat{ }5_DX without the v5 tag) |
| 212 | MEEQWAREIGAQLRRMADDLNAQYERRGSGVKQTLNFDLLKLAGDVESN |
| | PGPDMRPEIWIAQELRRIGDEFNAYYARRGSGVKQTLNFDLLKLAGDVESN |
| | PGPEEQWAREIGAQLRRMADDLNAQYERRGSGVKQTLNFDLLKLAGDVES |
| | NPGPDMRPEIWIAQELRRIGDEFNAYYARRGSGVKQTLNFDLLKLAGDVEN |
| | PGPEEQWAREIGAQLRRMADDLNAQYERRGSGVKQTLNFDLLKLAGDVES |
| | NPGPDMRPEIWIAQERRIGDEFNAYYARRGSGVKQTLNFDLLKLAGDVESN |
| | PGPEEQWAREIGAQLRRMADDLNAQYERRGSGVKQTLNFDLLKLAGDVES |
| | NPGPDMRPEIWIAQELRRIGDEFNAYYARRGSGVKQTLNFDLLKLAGDVES |
| | NPGPEEQWAREIGAQLRRMADDLNAQYERRGSGVKQTLNFDLLKLAGDVE |
| | SNPGPDMRPEIWIAQELRRIGDEFNAYYARRGSGVKQTLNFDLLKLAGDVE |
| | SNPGPEEQWAREIGAQLRRMADDLNAQYERRGSGVKQTLNFDLLKLAGDV |
| | ESNPGPDMRPEIWIAQELRRIGDEFNAYYARRGSGVKQTLNFDLLKLAGDV |
| | ESNPGPEEQWAREIGAQLRRMADDLNAQYERRGSGVKQTLNFDLLKLAGD |
| | VESNPGPDMRPEIWIAQELRRIGDEFNAYYARRGSGVKQTLNFDLLKLAGD |
| | VESNPGPEEQWAREIGAQLRRMADDLNAQYERRGSGVKQTLNFDLLKLAG |
| | DVESNPGPDMRPEIWIAQELRRIGDEFNAYYARRGSGVKQTLNFDLLKLAG |
| | DVESNPGPEEQWAREIGAQLRRMADDLNAQYERRGSGVKQTLNFDLLKLA |
| | GDVESNPGPDMRPEIWIAQELRRIGDEFNAYYARRGSGVKQTLNFDLLKLA |
| | GDVESNPGPEEQWAREIGAQLRRMADDLNAQYERRGSGVKQTLNFDLLKL |
| | AGDVESNPGPDMRPEIWIAQELRRIGDEFNAYYARRGSGVKQTLNFDLLKL |
| | AGDVESNPGP |
| | ((Puma-F2A-BimBH3-F2A)\hat{ }10 without the v5 tag) |
| 213 | MQWAREIGAQLRRIGDDLNAQYERRRQGSGVKQTLNFDLLKLAGDVESNP |
| | GPQWAREIGAQLRRIGDDLNAQYERRRQGSGVKQTLNFDLLKLAGDVESN |
| | PGPQWAREIGAQLRRIGDDLNAQYERRRQGSGVKQTLNFDLLKLAGDVES |
| | NPGPQWAREIGAQLRRIGDDLNAQYERRRQGSGVKQTLNFDLLKLAGDES |
| | NPGPQWAREIGAQLRRIGDDLNAQYERRRQGSGVKQTLNFDLLKLAGDVE |
| | SNPGPQWAREIGAQLRRIGDDLNAQYERRRQGSGVKQTLNFDLLKLAGDV |
| | ESNPGPQWAREIGAQLRRIGDDLNAQYERRRQGSGVKQTLNFDLLKLAGD |
| | VESNPGPQWAREIGAQLRRIGDDLNAQYERRRQGSGVKQTLNFDLLKLAG |
| | DVESNPGPQWAREIGAQLRRIGDDLNAQYERRRQGSGVKQTLNFDLLKLA |
| | GDVESNPGPQWAREIGAQLRRIGDDLNAQYERRRQGSGVKQTLNFDLLKL |
| | AGDVESNPGP |
| | ((SuperPumaBH3.F2A)\hat{ }10, (SuperPumaBH3.F2A)\hat{ }10_DX without the v5 tag) |
| 214 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESN |
| | PGPQWAREIGAQERREADDENAQYERRRQGSGVKQTLNFDLLKLAGDVES |
| | NPGPQWAREIGAQERREADDENAQYERRRQGSGVKQTLNFDLLKLAGDVE |
| | SNPGPQWAREIGAQERREADDENAQYERRRQGSGVKQTLNFDLLKLAGDV |
| | ESNPGPQWAREIGAQERREADDENAQYERRRQGSGVKQTLNFDLLKLAGD |
| | VESNPGPQWAREIGAQERREADDENAQYERRRQGSGVKQTLNFDLLKLAG |
| | DVESNPGPQWAREIGAQERREADDENAQYERRRQGSGVKQTLNFDLLKLA |
| | GDVESNPGPQWAREIGAQERREADDENAQYERRRQGSGVKQTLNFDLLKL |
| | AGDVESNPGPQWAREIGAQERREADDENAQYERRRQGSGVKQTLNFDLLK |
| | LAGDVESNPGPQWAREIGAQERREADDENAQYERRRQGSGVKQTLNFDLL |
| | KLAGDVESNPGP |
| | ((KittyCatBH3.F2A)\hat{ }10, (KittyCatBH3.F2A)\hat{ }10_DX without the v5 tag) |
| 215 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESN |
| | PGPQWAREIGAQERREADDENAQYERRRQGSGVKQTLNFDLLKLAGDVES |
| | NPGPQWAREIGAQERREADDENAQYERRRQGSGVKQTLNFDLLKLAGDVE |
| | SNPGPQWAREIGAQERREADDENAQYERRRQGSGVKQTLNFDLLKLAGDV |
| | ESNPGPQWAREIGAQERREADDENAQYERRRQGSGVKQTLNFDLLKLAGD |
| | VESNPGPQWAREIGAQERREADDNAQYERRRQGSGVKQTLNFDLLKLAGD |
| | VESNPGPQWAREIGAQERREADDENAQYERRRQGSGVKQTLNFDLLKLAG |
| | DVESNPGPQWAREIGAQERREADDENAQYERRRQGSGVKQTLNFDLLKLA |
| | GDVESNPGPQWAREIGAQERREADDENAQYERRRQGSGVKQTLNFDLLKL |
| | AGDVESNPGPQWAREIGAQERREADDENAQYERRRQGSGVKQTLNFDLLK |
| | LAGDVESNPGP |
| | ((KittyCatBH3.F2A)\hat{ }10, (KittyCatBH3.F2A)\hat{ }10_DX without the v5 tag) |
| 216 | MPAELEVECATQLRRFGDKLNFRQKLLGSGVKQTLNFDLLKLAGDVESNPGP |
| | PAELEVECATQLRRFGDKLNFRQKLLGSGVKQTLNFDLLKLAGDVESNPGPP |
| | AELEVECATQLRRFGDKLNFRQKLLGSGVKQTLNFDLLKLAGDVESNPGPPA |
| | ELEVECATQLRRFGDKLNFRQKLLGSGVKQTLNFDLLKLAGDVESNPGPPAE |
| | LEVECATQLRRFGDKLNFRQKLLGSGVKQTLNFDLLKLAGDVESNPGPPAEL |
| | EVECATQLRRFGDKLNFRQKLLGSGVKQTLNFDLLKLAGDVESNPGP |
| | ((NoxaBH3.F2A)\hat{ }5, (NoxaBH3.F2A)\hat{ }5_DX without the v5 tag) |
| 217 | MPAELEVECATQLRRFGDKLNFRQKLLGSGVKQTLNFDLLKLAGDVESNPGP |
| | PAELEVECATQLRRFGDKLNFRQKLLGSGVKQTLNFDLLKLAGDVESNPGPP |
| | AELEVECATQLRRFGDKLNFRQKLLGSGVKQTLNFDLLKLAGDVESNPGPPA |
| | ELEVECATQLRRFGDKLNFRQKLLGSGVKQTLNFDLLKLAGDVESNPGPPAE |
| | LEVECATQLRRFGDKLNFRQKLLGSGVKQTLNFDLLKLAGDVESNPGPPAEL |
| | EVECATQLRRFGDKLNFRQKLLGSGVKQTLNFDLLKLAGDVESNPGPPAELE |
| | VECATQLRRFGDKLNFRQKLLGSGVKQTLNFDLLKLAGDVESNPGPPAELEV |
| | ECATQLRRFGDKLNFRQKLLGSGVKQTLNFDLLKLAGDVESNPGPPAELEVE |
| | CATQLRRFGDKLNFRQKLLGSGVKQTLNFDLLKLAGDVESNPGPPAELEVEC |
| | ATQLRRFGDKLNFRQKLLGSGVKQTLNFDLLKLAGDVESNPGPPAELEVECA |
| | TQLRRFGDKLNFRQKLLGSGVKQTLNFDLLKLAGDVESNPGP |
| | ((NoxaBH3.F2A)\hat{ }10, (NoxaBH3.F2A)\hat{ }10_DX without the v5 tag) |
| 218 | MNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GPNLWAAQRYGRELRRMSDEFVDSFKKGGSGVKQTLNFDLLKLAGDVESNP |
| | GP |
| | ((BadBH3.F2A)\hat{ }10,(BadBH3.F2A)\hat{ }10_DX without the v5 tag) |
| 219 | MPAELEVECATQLRRFGDKLNFRQKLLGSGVKQTLNFDLLKLAGDVESNPGP |
| | NLWAAQRYGRELRRMSDEFVDSFKKGGSGVKQTLNFDLLKLAGDVESNPGP |
| | PAELEVECATQLRRFGDKLNFRQKLLGSGVKQTLNFDLLKLAGDVESNPGPN |
| | LWAAQRYGRELRRMSDEFVDSFKKGGSGVKQTLNFDLLKLAGDVESNPGPP |
| | AELEVECATQLRRFGDKLNFRQKLLGSGVKQTLNFDLLKLAGDVESNPGPNL |
| | WAAQRYGRELRRMSDEFVDSFKKGGSGVKQTLNFDLLKLAGDVESNPGPPA |
| | ELEVECATQLRRFGDKLNFRQKLLGSGVKQTLNFDLLKLAGDVESNPGPNL |
| | WAAQRYGRELRRMSDEFVDSFKKGGSGVKQTLNFDLLKLAGDVESNPGPPA |
| | ELEVECATQLRRFGDKLNFRQKLLGSGVKQTLNFDLLKLAGDVESNPGPNL |
| | WAAQRYGRELRRMSDEFVDSFKKGGSGVKQTLNFDLLKLAGDVESNPGP |
| | ((Noxa-F2A-BadBH3-F2A)\hat{ }5, (Noxa-F2A-BadBH3-F2A)\hat{ }5_DX without the v5 tag) |
| 220 | MPAELEVECATQLRRFGDKLNFRQKLLGSGVKQTLNFDLLKLAGDVESNPG |
| | PNLWAAQRYGRELRRMSDEFVDSFKKGGSGVKQTLNFDLLKLAGDVESNP |
| | GPPAELEVECATQLRRFGDKLNFRQKLLGSGVKQTLNFDLLKLAGDVESNP |
| | GPNLWAAQRYGRELRRMSDEFVDSFKKGGSGVKQTLNFDLLKLAGDVESN |
| | GPPAELEVECATQLRRFGDKLNFRQKLLGSGVKQTLNFDLLKLAGDVESNP |
| | GPNLWAAQRYGRELRRMSDEFVDSFKKGGSGVKQTLNFDLLKLAGDVESN |
| | PGPPAELEVECATQLRRFGDKLNFRQKLLGSGVKQTLNFDLLKLAGDVESN |
| | PGPNLWAAQRYGRELRRMSDEFVDSFKKGGSGVKQTLNFDLLKLAGDVES |
| | NPGPPAELEVECATQLRRFGDKLNFRQKLLGSGVKQTLNFDLLKLAGDVES |
| | NPGPNLWAAQRYGRELRRMSDEFVDSFKKGGSGVKQTLNFDLLKLAGDVE |
| | SNPGPPAELEVECATQLRRFGDKLNFRQKLLGSGVKQTLNFDLLKLAGDVE |
| | SNPGPNLWAAQRYGRELRRMSDEFVDSFKKGGSGVKQTLNFDLLKLAGDV |
| | ESNPGPPAELEVECATQLRRFGDKLNFRQKLLGSGVKQTLNFDLLKLAGDV |
| | ESNPGPNLWAAQRYGRELRRMSDEFVDSFKKGGSGVKQTLNFDLLKLAGD |
| | VESNPGPPAELEVECATQLRRFGDKLNFRQKLLGSGVKQTLNFDLLKLAGD |
| | VESNPGPNLWAAQRYGRELRRMSDEFVDSFKKGGSGVKQTLNFDLLKLAG |
| | DVESNPGPPAELEVECATQLRRFGDKLNFRQKLLGSGVKQTLNFDLLKLAG |
| | DVESNPGPNLWAAQRYGRELRRMSDEFVDSFKKGGSGVKQTLNFDLLKLA |
| | GDVESNPGPPAELEVECATQLRRFGDKLNFRQKLLGSGVKQTLNFDLLKLA |
| | GDVESNPGPNLWAAQRYGRELRRMSDEFVDSFKKGGSGVKQTLNFDLLKL |
| | AGDVESNPGP |
| | ((Noxa-F2A-BadBH3-F2A)\hat{ }10 without the v5 tag) |
| 221 | MEEQWAREIGAQLRRMADDLNAQYERRGSGVKQTLNFDLLKLAGDVESNP |
| | GPDMRPEIWIAQELRRIGDEFNAYYARRGSGVKQTLNFDLLKLAGDVESNPG |
| | PEEQWAREIGAQLRRMADDLNAQYERRGSGVKQTLNFDLLKLAGDVESNPG |
| | PDMRPEIWIAQELRRIGDEFNAYYARRGSGVKQTLNFDLLKLAGDVESNPGP |
| | EEQWAREIGAQLRRMADDLNAQYERRGSGVKQTLNFDLLKLAGDVESNPGP |
| | DMRPEIWIAQELRRIGDEFNAYYARRGSGVKQTLNFDLLKLAGDVESNPGP |
| | ((Puma-F2A-BimBH3-F2A)\hat{ }3_DX without the v5 tag) |
| 222 | MQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PGSGVKQTLNFDLLKLAGDVESNPGP |
| | ((SuperPumaBH3.F2A)\hat{ }3_DNA2.0 without the v5 tag) |
| 223 | MEEQWAREIGAQLRRMADDLNAQYERRGSGVKQTLNFDLLKLAGDVESNP |
| | GPDMRPEIWIAQELRRIGDEFNAYYARRGSGVKQTLNFDLLKLAGDVESNPG |
| | PEEQWAREIGAQLRRMADDLNAQYERRGSGVKQTLNFDLLKLAGDVESNPG |
| | PDMRPEIWIAQELRRIGDEFNAYYARRGSGVKQTLNFDLLKLAGDVESNPGP |
| | EEQWAREIGAQLRRMADDLNAQYERRGSGVKQTLNFDLLKLAGDVESNPGP |
| | DMRPEIWIAQELRRIGDEFNAYYARRGSGVKQTLNFDLLKLAGDVESNPGP |
| | ((Puma-F2A-BimBH3-F2A)\hat{ }3_DX DNA2.0 without the v5 tag) |
| 224 | MQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | PQWAREIGAQLRRIGDDLNAQYERRRQGSGVKQTLNFDLLKLAGDVESNPG |
| | P |
| | ((SuperPumaBH3.F2A)\hat{ }5_DNA2.0 without the v5 tag) |
| 225 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GP |
| | ((pumaBH3.F2A)\hat{ }5_DNA2.0 without the v5 tag) |
| 226 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESN |
| | PGPQWAREIGAQERREADDENAQYERRRQGSGVKQTLNFDLLKLAGDVES |
| | NPGPQWAREIGAQERREADDENAQYERRRQGSGVKQTLNFDLLKLAGDVE |
| | SNPGPQWAREIGAQERREADDENAQYERRRQGSGVKQTLNFDLLKLAGDV |
| | ESNPGP |
| | ((KittyCatBH3.F2A)\hat{ }5_DNA2.0 without the v5 tag) |
| 227 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESN |
| | PGPQWAREIGAQERREADDENAQYERRRQGSGVKQTLNFDLLKLAGDVES |
| | NPGPQWAREIGAQERREADDENAQYERRRQGSGVKQTLNFDLLKLAGDVE |
| | SNPGPQWAREIGAQERREADDENAQYERRRQGSGVKQTLNFDLLKLAGDV |
| | ESNPGPQWAREIGAQERREADDENAQYERRRQGSGVKQTLNFDLLKLAGD |
| | VESNPGPQWAREIGAQERREADDENAQYERRRQGSGVKQTLNFDLLKLAG |
| | DVESNPGPQWAREIGAQERREADDENAQYERRRQGSGVKQTLNFDLLKLA |
| | GDVESNPGPQWAREIGAQERREADDENAQYERRRQGSGVKQTLNFDLLKL |
| | AGDVESNPGPQWAREIGAQERREADDENAQYERRRQGSGVKQTLNFDLLK |
| | LAGDVESNPGPQWAREIGAQERREADDENAQYERRRQGSGVKQTLNFDLL |
| | KLAGDVESNPGP |
| | (KittyCatBH3.F2A)\hat{ }10_DNA2.0 without the v5 tag) |
| 228 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GP |
| | ((pumaBH3.F2A)\hat{ }10_DNA2.0 without the v5 tag) |
| 229 | MEEQWAREIGAQLRRMADDLNAQYERRGSGVKQTLNFDLLKLAGDVESNP |
| | GPDMRPEIWIAQELRRIGDEFNAYYARRGSGVKQTLNFDLLKLAGDVESNPG |
| | PEEQWAREIGAQLRRMADDLNAQYERRGSGVKQTLNFDLLKLAGDVESNPG |
| | PDMRPEIWIAQELRRIGDEFNAYYARRGSGVKQTLNFDLLKLAGDVESNPGP |
| | EEQWAREIGAQLRRMADDLNAQYERRGSGVKQTLNFDLLKLAGDVESNPGP |
| | DMRPEIWIAQELRRIGDEFNAYYARRGSGVKQTLNFDLLKLAGDVESNPGPE |
| | EQWAREIGAQLRRMADDLNAQYERRGSGVKQTLNFDLLKLAGDVESNPGPD |
| | MRPEIWIAQELRRIGDEFNAYYARRGSGVKQTLNFDLLKLAGDVESNPGPEE |
| | QWAREIGAQLRRMADDLNAQYERRGSGVKQTLNFDLLKLAGDVESNPGPD |
| | MRPEIWIAQELRRIGDEFNAYYARRGSGVKQTLNFDLLKLAGDVESNPGP |
| | ((Puma-F2A-BimBH3-F2A)\hat{ }5_DNA2.0 without the v5 tag) |
| 230 | MQWAREIGAQLRRIGDDLNAQYERRRQGSGVKQTLNFDLLKLAGDVESNP |
| | GPQWAREIGAQLRRIGDDLNAQYERRRQGSGVKQTLNFDLLKLAGDVESN |
| | PGPQWAREIGAQLRRIGDDLNAQYERRRQGSGVKQTLNFDLLKLAGDVES |
| | NPGPQWAREIGAQLRRIGDDLNAQYERRRQGSGVKQTLNFDLLKLAGDVE |
| | SNPGPQWAREIGAQLRRIGDDLNAQYERRRQGSGVKQTLNFDLLKLAGDV |
| | ESNPGPQWAREIGAQLRRIGDDLNAQYERRRQGSGVKQTLNFDLLKLAGD |
| | VESNPGPQWAREIGAQLRRIGDDLNAQYERRRQGSGVKQTLNFDLLKLAG |
| | DVESNPGPQWAREIGAQLRRIGDDLNAQYERRRQGSGVKQTLNFDLLKLA |
| | GDVESNPGPQWAREIGAQLRRIGDDLNAQYERRRQGSGVKQTLNFDLLKL |
| | AGDVESNPGPQWAREIGAQLRRIGDDLNAQYERRRQGSGVKQTLNFDLLK |
| | LAGDVESNPGP |
| | ((SuperPumaBH3.F2A)\hat{ }10_DNA2.0 without the v5 tag) |
| 231 | MQWAREIGAQERREADDENAQYERRRQGSGVKQTLNFDLLKLAGDVESNP |
| | GPQWAREIGAQERREADDENAQYERRRQGSGVKQTLNFDLLKLAGDVESNP |
| | GPQWAREIGAQERREADDENAQYERRRQGSGVKQTLNFDLLKLAGDVESNP |
| | GP |
| | ((KittyCatBH3.F2A)\hat{ }3 without the v5 tag) |
| 232 | MQWAREIGAQERREADDENAQYERRRQGSGATNFSLLKQAGDVEENPGPQ |
| | WAREIGAQERREADDENAQYERRRQGSGATNFSLLKQAGDVEENPGPQWA |
| | REIGAQERREADDENAQYERRRQGSGVKQTLNFDLLKLAGDVESNPGP |
| | ((KittyCatBH3.P2A)\hat{ }3 without the v5 tag) |
| 233 | MEEQWAREIGAQLRRMADDLNAQYERRGSGATNFSLLKQAGDVEENPGPEE |
| | QWAREIGAQLRRMADDLNAQYERRGSGATNFSLLKQAGDVEENPGPEEQW |
| | AREIGAQLRRMADDLNAQYERR |
| | (PumaBH3(x3.P2A)_miR122 without the v5 tag) |
| 234 | MEEQWAREIGAQLRRMADDLNAQYERRGGGSGGGSGGGSEEQWAREIGAQ |
| | LRRMADDLNAQYERRGGGSGGGSGGGSEEQWAREIGAQLRRMADDLNAQY |
| | ERR |
| | ((PUMA BH3.GGGSx3)x3 without the v5 tag) |
| 235 | MEEQWAREIGAQLRRMADDLNAQYERRGGGSGGGSGGGSEEQWAREIGAQ |
| | LRRMADDLNAQYERRGGGSGGGSGGGSEEQWAREIGAQLRRMADDLNAQY |
| | ERR |
| | ((PUMA BH3.GGGSx3)x3_DX without the v5 tag) |
| 236 | MEEQWAREIGAQLRRMADDLNAQYERRGGGSGGGSGGGSEEQWAREIGAQ |
| | LRRMADDLNAQYERR (PUMA BH3.GGGSx3.PUMA BH3 without the v5 tag) |
| 237 | MEEQWAREIGAQLRRMADDLNAQYERRGSGATNFSLLKQAGDVEENPGPEE |
| | QWAREIGAQLRRMADDLNAQYERRGSGATNFSLLKQAGDVEENPGPEEQW |
| | AREIGAQLRRMADDLNAQYERR |
| | (PumaBH3(x3.P2A)_miR122 without the v5 tag) |
| 238 | ATGGAGGAGCAGTGGGCTCGAGAGATCGGAGCCCAATTGAGGAGGATGG |
| | CGGATGACCTCAATGCACAGTACGAACGTAGG |
| | (PumaBH3_HS3UPCRfree) |
| 239 | ATGCACCATCATCATCACCACGAAGAGCAATGGGCGCGGGAAATTGGAG |
| | CCCAGCTGCGGAGAATGGCTGATGATTTGAACGCGCAGTATGAAAGACGT |
| | (PumaBH3.nHIS_HS3UPCRfree) |
| 240 | ATGGGTAAGCCTATCCCAAACCCTCTGCTGGGGCTCGACTCTACAGAGGA |
| | ACAATGGGCACGAGAGATCGGGGCTCAGTTACGACGTATGGCGGACGAC |
| | CTGAACGCCCAGTATGAGCGAAGG |
| | (PumaBH3.nV5_HS3UPCRfree) |
| 241 | ATGGAGGAACAGTGGGCAAGAGAGATTGGCGCGCAGTTGAGGAGAATGG |
| | CAGATGACCTGAATGCTCAATATGAGAGACGGCACCATCATCATCATCAT |
| | (PumaBH3.cHIS_HS3UPCRfree) |
| 242 | ATGGAAGAACAGTGGGCAAGAGAAATCGGAGCACAGCTGCGACGGATG |
| | GCCGACGACTTGAACGCGCAGTATGAAAGAAGAGGAAGCGGCGTGAAA |
| | CAGACACTGAACTTCGACCTTCTGAAACTGGCAGGTGATGTGGAATCAA |
| | ATCCTGGTCCCGAAGAGCAATGGGCCAGAGAGATCGGCGCCCAGCTTCG |
| | GCGCATGGCCGACGATCTTAATGCCCAGTACGAACGGCGGGGGTCTGGA |
| | GTGAAACAAACTCTGAACTTCGATCTCCTGAAACTAGCGGGGGACGTTG |
| | AGTCCAACCCGGGGCCTGAGGAACAATGGGCCCGCGAGATCGGCGCCC |
| | AATTGAGACGTATGGCCGACGATCTAAATGCCCAGTACGAACGGCGCGG |
| | CAAGCCTATTCCAAATCCGTTGTTGGGGCTGGACAGCACA |
| | (PumaBH3(x3.F2A).v5_Hs3UPCRfree) |
| 243 | ATGGAGGAGCAGTGGGCTAGGGAAATAGGCGCCCAGCTCAGACGGATGG |
| | CGGACGATCTCAATGCACAATACGAAAGGCGGGGAAAGCCAATCCCAAA |
| | TCCGCTCCTGGGGTTGGACAGTACT |
| | (PumaBH3.cV5_HS3UPCRfree) |
| 244 | ATGGAGGAGCAATGGGCTAGAGAGATCGGCGCACAGCTGCGGCGCATG |
| | GCCGATGATCTGAACGCCCAATACGAGAGGAGAGGTTCCGGAGTGAAG |
| | CAGACTCTGAACTTCGATCTGCTCAAGCTTGCGGGCGACGTGGAATCGA |
| | ACCCCGGCCCTGAGGAACAATGGGCGCGCGAAATCGGTGCCCAGCTCCG |
| | CCGGATGGCAGACGACCTGAACGCGCAGTACGAGCGGCGGGGGAGCGG |
| | GGTCAAGCAGACCCTGAATTTCGACCTTCTGAAGCTGGCCGGAGATGTG |
| | GAGTCAAACCCGGGACCCGAAGAACAGTGGGCCAGGGAAATTGGAGCT |
| | CAGCTGCGGAGAATGGCCGACGACCTCAACGCCCAGTACGAACGGCGC |
| | GGAAAACCTATCCCGAACCCACTCTTGGGCCTGGACTCCACC |
| | (PumaBH3(x3.F2A).v5_miR142.3p_tp) |
| 245 | ATGGAGGAGCAATGGGCTAGAGAGATCGGCGCACAGCTGCGGCGCATG |
| | GCCGATGATCTGAACGCCCAATACGAGAGGAGAGGTTCCGGAGTGAAG |
| | CAGACTCTGAACTTCGATCTGCTCAAGCTTGCGGGCGACGTGGAATCGA |
| | ACCCCGGCCCTGAGGAACAATGGGCGCGCGAAATCGGTGCCCAGCTCCG |
| | CCGGATGGCAGACGACCTGAACGCGCAGTACGAGCGGCGGGGGAGCGG |
| | GGTCAAGCAGACCCTGAATTTCGACCTTCTGAAGCTGGCCGGAGATGTG |
| | GAGTCAAACCCGGGACCCGAAGAACAGTGGGCCAGGGAAATTGGAGCT |
| | CAGCTGCGGAGAATGGCCGACGACCTCAACGCCCAGTACGAACGGCGC |
| | GGAAAACCTATCCCGAACCCACTCTTGGGCCTGGACTCCACC |
| | (PumaBH3(x3.F2A).v5_miR122/142.3p_tp) |
| 246 | ATGGAGGAGCAATGGGCTAGAGAGATCGGCGCACAGCTGCGGCGCATG |
| | GCCGATGATCTGAACGCCCAATACGAGAGGAGAGGTTCCGGAGTGAAG |
| | CAGACTCTGAACTTCGATCTGCTCAAGCTTGCGGGCGACGTGGAATCGA |
| | ACCCCGGCCCTGAGGAACAATGGGCGCGCGAAATCGGTGCCCAGCTCCG |
| | CCGGATGGCAGACGACCTGAACGCGCAGTACGAGCGGCGGGGGAGCGG |
| | GGTCAAGCAGACCCTGAATTTCGACCTTCTGAAGCTGGCCGGAGATGTG |
| | GAGTCAAACCCGGGACCCGAAGAACAGTGGGCCAGGGAAATTGGAGCT |
| | CAGCTGCGGAGAATGGCCGACGACCTCAACGCCCAGTACGAACGGCGC |
| | GGAAAACCTATCCCGAACCCACTCTTGGGCCTGGACTCCACC |
| | (PumaBH3(x3.F2A).v5_tp) |
| 247 | ATGGAGGAGCAATGGGCTAGAGAGATCGGCGCACAGCTGCGGCGCATG |
| | GCCGATGATCTGAACGCCCAATACGAGAGGAGAGGTTCCGGAGTGAAG |
| | CAGACTCTGAACTTCGATCTGCTCAAGCTTGCGGGCGACGTGGAATCGA |
| | ACCCCGGCCCTGAGGAACAATGGGCGCGCGAAATCGGTGCCCAGCTCCG |
| | CCGGATGGCAGACGACCTGAACGCGCAGTACGAGCGGCGGGGGAGCGG |
| | GGTCAAGCAGACCCTGAATTTCGACCTTCTGAAGCTGGCCGGAGATGTG |
| | GAGTCAAACCCGGGACCCGAAGAACAGTGGGCCAGGGAAATTGGAGCT |
| | CAGCTGCGGAGAATGGCCGACGACCTCAACGCCCAGTACGAACGGCGC |
| | GGAAAACCTATCCCGAACCCACTCTTGGGCCTGGACTCCACC |
| | (PumaBH3(x3.F2A).v5_miR122_tp) |
| 248 | ATGGAAGAACAATGGGCACGGGAAATTGGAGCGCAGCTGAGGAGAATG |
| | GCTGATGACCTGAACGCTCAATACGAACGCCGCGGATCCGGCGTGAAGC |
| | AGACTCTTAACTTCGACTTGCTCAAGCTCGCTGGCGATGTCGAGTCCAAC |
| | CCGGGCCCCGAGGAGCAATGGGCCCGCGAAATTGGGGCCCAGCTCCGGC |
| | GCATGGCGGACGATCTCAACGCGCAGTATGAACGGAGGGGCTCCGGAGT |
| | CAAGCAGACCCTCAACTTCGACCTCCTGAAGCTCGCCGGCGACGTGGAG |
| | AGCAACCCTGGTCCCGAAGAACAGTGGGCCAGAGAAATCGGTGCCCAG |
| | CTTCGCCGGATGGCCGACGACTTGAACGCCCAATACGAGCGGAGAGGCT |
| | CGGGAGTGAAGCAAACCCTGAACTTCGATCTGCTTAAGCTGGCTGGGGA |
| | CGTCGAATCCAACCCCGGACCTGAGGAGCAGTGGGCCCGGGAAATCGG |
| | GGCCCAACTGAGAAGGATGGCAGACGACCTGAATGCCCAGTACGAGCG |
| | CCGCGGCTCAGGAGTGAAACAGACCTTGAACTTTGACTTGCTGAAGCTG |
| | GCCGGGGACGTGGAAAGCAACCCGGGGCCAGAGGAACAGTGGGCGCGG |
| | GAGATCGGTGCTCAGCTGAGAAGAATGGCAGATGATCTGAATGCGCAGT |
| | ACGAACGGCGCGGATCTGGAGTCAAACAGACCCTGAATTTCGACCTGTT |
| | GAAACTGGCCGGAGATGTGGAATCAAACCCCGGCCCTGGAAAGCCGATC |
| | CCGAACCCACTGCTGGGGCTCGACTCCACC |
| | ((pumaBH3.F2A)\hat{ }5_cV5, (pumaBH3.F2A)\hat{ }5_cV5_DX) |
| 249 | ATGGACATGCGACCTGAGATTTGGATTGCGCAGGAACTTAGACGGATTG |
| | GCGATGAATTCAACGCATACTATGCACGGAGAGGCTCCGGCGTCAAGCA |
| | GACCCTGAACTTTGACCTCCTCAAGCTGGCGGGCGATGTGGAGAGCAAC |
| | CCCGGCCCCGATATGCGCCCAGAAATCTGGATTGCTCAAGAACTCCGCA |
| | GAATCGGCGACGAGTTTAATGCGTACTACGCCAGACGCGGAAGCGGCGT |
| | GAAGCAGACTCTTAACTTCGATCTGCTCAAACTGGCCGGCGACGTGGAG |
| | TCAAATCCTGGCCCGGATATGCGGCCCGAGATATGGATTGCCCAGGAGC |
| | TCCGCCGCATCGGGGACGAGTTCAACGCCTACTACGCACGCCGCGGGTC |
| | GGGCGTGAAACAGACCTTGAACTTTGATCTGCTGAAGCTGGCAGGGGAC |
| | GTGGAATCCAACCCGGGACCGGACATGAGGCCCGAGATCTGGATCGCCC |
| | AGGAACTGCGGAGAATCGGAGATGAGTTTAACGCCTATTACGCCCGGAG |
| | GGGATCGGGAGTCAAGCAAACCCTCAACTTCGACCTGTTGAAGCTGGCT |
| | GGAGATGTCGAATCGAACCCCGGTCCGGACATGCGGCCGGAAATTTGGA |
| | TCGCACAGGAACTGAGGAGGATTGGGGACGAATTCAATGCTTACTACGC |
| | CCGCCGCGGATCCGGAGTGAAGCAAACACTGAATTTCGACCTCCTGAAG |
| | CTTGCCGGGGACGTCGAGTCCAACCCTGGTCCTGGAAAGCCGATCCCGA |
| | ACCCTTTGCTGGGCCTGGACAGCACC |
| | ((BimBH3.F2A)\hat{ }5_cV5, (BimBH3.F2A)\hat{ }5_cV5_DX) |
| 250 | ATGCAATGGGCCAGAGAGATCGGAGCACAACTGCGGAGAATTGGAGAT |
| | GACCTGAACGCACAGTACGAACGCCGCAGACAGGGATCGGGCGTGAAG |
| | CAAACCCTCAATTTCGATCTTCTGAAATTGGCGGGCGACGTCGAGTCGA |
| | ACCCTGGTCCACAATGGGCGCGGGAAATCGGTGCCCAGCTGAGGAGAAT |
| | TGGCGATGACCTCAACGCCCAGTACGAAAGGCGGCGGCAGGGCAGCGG |
| | AGTGAAGCAGACTCTTAACTTCGACTTGCTGAAGCTGGCTGGGGACGTG |
| | GAATCCAACCCTGGACCCCAGTGGGCCCGCGAAATCGGCGCCCAGCTGC |
| | GGCGCATCGGGGACGACCTCAATGCGCAGTACGAGCGGCGGCGCCAGG |
| | GGTCCGGCGTCAAGCAGACCCTGAACTTCGACCTCCTCAAGCTGGCCGG |
| | AGATGTGGAGTCCAACCCGGGTCCCGGATCAGGGGTCAAGCAAACTCTG |
| | AACTTTGATCTGCTGAAGCTCGCTGGCGACGTGGAGAGCAACCCGGGAC |
| | CGGGAAAGCCTATTCCGAACCCCCTGCTGGGCCTGGACTCCACC |
| | ((SuperPumaBH3.F2A)\hat{ }3_cV5) |
| 251 | ATGCAATGGGCCAGAGAAATTGGAGCGCAACTCAGAAGAATCGGCGAC |
| | GATCTGAACGCTCAGTACGAAAGACGCCGCCAGGGATCCGGCGTGAAGC |
| | AAACTCTGAACTTTGACCTCCTTAAGCTCGCCGGGGATGTCGAGAGCAA |
| | TCCTGGACCGCAGTGGGCCAGGGAGATCGGTGCCCAGTTGCGGCGGATC |
| | GGCGATGATCTCAACGCCCAATATGAACGGCGGAGACAGGGAAGCGGA |
| | GTCAAGCAAACCTTGAACTTCGACCTCCTGAAGCTGGCTGGCGACGTCG |
| | AATCCAACCCTGGACCCCAGTGGGCGCGGGAAATTGGTGCCCAGCTGCG |
| | ACGGATCGGGGATGACCTCAACGCACAATACGAGAGGCGGAGGCAGGG |
| | TTCCGGCGTCAAGCAGACTCTTAATTTCGATCTCCTGAAATTGGCCGGCG |
| | ATGTGGAGTCAAACCCGGGCCCGCAATGGGCCCGCGAGATCGGCGCCCA |
| | ACTGCGGCGCATTGGCGACGACCTGAACGCCCAGTACGAGCGCAGACGC |
| | CAGGGGTCCGGAGTGAAGCAGACCCTCAACTTCGATTTGTTGAAGCTCG |
| | CGGGCGACGTGGAAAGCAATCCGGGACCACAGTGGGCTAGAGAGATCG |
| | GAGCCCAGCTGAGGCGCATTGGTGACGACCTCAATGCACAGTACGAACG |
| | GAGGCGGCAGGGTTCGGGGGTGAAACAGACCCTGAACTTCGATCTGCTC |
| | AAGCTGGCGGGAGATGTGGAATCGAACCCAGGGCCGGGAAAGCCTATT |
| | CCCAACCCTCTGCTGGGCCTGGATTCCACC |
| | ((SuperPumaBH3.F2A)\hat{ }5_cV5) |
| 252 | ATGCAATGGGCACGAGAGATTGGAGCACAGGAACGGAGAGAAGCCGAT |
| | GACGAGAACGCGCAGTACGAACGGCGGCGCCAGGGTTCCGGCGTGAAA |
| | CAGACTCTCAACTTCGACTTGCTGAAGCTCGCCGGGGACGTGGAATCCA |
| | ATCCAGGACCTCAATGGGCCCGGGAAATCGGCGCGCAGGAAAGACGCG |
| | AAGCCGACGATGAAAACGCCCAATACGAGCGGCGCAGACAGGGGTCGG |
| | GCGTGAAGCAAACGCTGAATTTCGACCTCCTGAAGTTGGCCGGAGATGT |
| | CGAGTCCAACCCTGGACCCCAGTGGGCCAGAGAGATTGGTGCCCAAGAA |
| | AGGAGAGAAGCGGATGACGAAAACGCTCAGTACGAGAGGCGCAGACAA |
| | GGCTCGGGAGTCAAGCAGACTCTGAACTTCGATCTTCTGAAGCTGGCTG |
| | GCGATGTGGAAAGCAACCCAGGACCGCAGTGGGCGCGCGAGATCGGAG |
| | CCCAGGAGAGGAGGGAGGCCGACGACGAAAATGCACAGTACGAAAGAA |
| | GGCGCCAGGGCTCCGGAGTGAAGCAGACCCTCAACTTTGACTTGCTCAA |
| | GCTGGCCGGCGACGTGGAGAGCAACCCTGGCCCGCAATGGGCTAGAGA |
| | AATCGGAGCTCAGGAGCGGCGGGAAGCAGACGACGAGAATGCGCAGTA |
| | TGAGCGCCGGCGGCAGGGAAGCGGTGTCAAGCAAACCCTGAACTTTGAT |
| | CTGCTCAAACTGGCCGGGGATGTGGAATCGAACCCCGGACCGGGGAAGC |
| | CCATCCCCAACCCGCTTCTGGGCCTGGACTCCACC |
| | ((KittyCatBH3.F2A)\hat{ }5_cV5,(KittyCatBH3.F2A)\hat{ }5_cV5_DX) |
| 253 | ATGCAATGGGCACGAGAGATTGGAGCACAGGAACGGAGAGAAGCCGATG |
| | ACGAGAACGCGCAGTACGAACGGCGGCGCCAGGGTTCCGGCGTGAAACA |
| | GACTCTCAACTTCGACTTGCTGAAGCTCGCCGGGGACGTGGAATCCAATC |
| | CAGGACCTCAATGGGCCCGGGAAATCGGCGCGCAGGAAAGACGCGAAGC |
| | CGACGATGAAAACGCCCAATACGAGCGGCGCAGACAGGGGTCGGGCGTG |
| | AAGCAAACGCTGAATTTCGACCTCCTGAAGTTGGCCGGAGATGTCGAGTC |
| | CAACCCTGGACCCCAGTGGGCCAGAGAGATTGGTGCCCAAGAAAGGAGA |
| | GAAGCGGATGACGAAAACGCTCAGTACGAGAGGCGCAGACAAGGCTCGG |
| | GAGTCAAGCAGACTCTGAACTTCGATCTTCTGAAGCTGGCTGGCGATGTG |
| | GAAAGCAACCCAGGACCGCAGTGGGCGCGCGAGATCGGAGCCCAGGAGA |
| | GGAGGGAGGCCGACGACGAAAATGCACAGTACGAAAGAAGGCGCCAGG |
| | GCTCCGGAGTGAAGCAGACCCTCAACTTTGACTTGCTCAAGCTGGCCGGC |
| | GACGTGGAGAGCAACCCTGGCCCGCAATGGGCTAGAGAAATCGGAGCTC |
| | AGGAGCGGCGGGAAGCAGACGACGAGAATGCGCAGTATGAGCGCCGGCG |
| | GCAGGGAAGCGGTGTCAAGCAAACCCTGAACTTTGATCTGCTCAAACTGG |
| | CCGGGGATGTGGAATCGAACCCCGGACCGGGGAAGCCCATCCCCAACCC |
| | GCTTCTGGGCCTGGACTCCACC |
| | ((KittyCatBH3.F2A)\hat{ }5_cV5, (KittyCatBH3.F2A)\hat{ }5_cV5_DX) |
| 254 | ATGAACCTCTGGGCAGCCCAACGCTACGGAAGAGAGCTTCGAAGAATGT |
| | CAGACGAGTTTGTCGACTCCTTCAAGAAAGGCGGTTCCGGAGTGAAGCA |
| | GACTCTGAATTTCGACCTTCTCAAGCTGGCCGGGGACGTGGAGAGCAAC |
| | CCTGGCCCAAATCTCTGGGCCGCTCAGCGGTACGGCCGGGAATTGAGAA |
| | GGATGTCGGATGAATTTGTGGATTCCTTCAAAAAGGGCGGATCCGGCGT |
| | GAAGCAAACTCTGAACTTCGACCTGTTGAAGCTTGCCGGCGACGTGGAG |
| | TCGAACCCCGGGCCGAACTTGTGGGCGGCTCAGAGATACGGTAGAGAAC |
| | TGCGGCGGATGAGCGACGAATTCGTGGACAGCTTTAAGAAGGGAGGAA |
| | GCGGAGTGAAACAAACCCTCAACTTCGATCTGCTGAAGCTCGCGGGCGA |
| | TGTCGAATCGAACCCGGGACCTAATCTGTGGGCTGCACAGCGCTACGGA |
| | CGCGAGCTGCGCCGCATGAGCGATGAGTTCGTGGACTCTTTTAAAAAGG |
| | GTGGTTCCGGCGTCAAACAGACTTTGAACTTTGACCTCCTGAAGCTTGCA |
| | GGGGATGTGGAATCCAACCCGGGGCCTAACCTGTGGGCGGCCCAAAGAT |
| | ACGGTCGGGAGCTGAGGCGCATGTCCGATGAATTCGTCGATTCGTTCAA |
| | GAAGGGGGGAAGCGGCGTGAAACAGACCCTTAACTTCGACTTGCTCAAA |
| | CTGGCGGGAGATGTGGAGTCCAACCCAGGCCCCGGAAAGCCGATCCCTA |
| | ATCCGCTGCTCGGCCTGGACTCCACC |
| | ((BadBH3.F2A)\hat{ }5_cV5) |
| 255 | ATGCCTGCCGAACTTGAAGTCGAATGCGCGACCCAGCTGAGAAGATTTG |
| | GAGACAAACTGAACTTCCGGCAAAAGCTGCTGGGCTCGGGAGTCAAACA |
| | GACTCTTAACTTCGACTTGCTGAAGCTTGCCGGCGACGTCGAGTCCAACC |
| | CGGGTCCAAACCTCTGGGCCGCCCAAAGATACGGACGGGAGCTCAGGA |
| | GAATGTCCGATGAGTTCGTGGATTCGTTCAAGAAGGGAGGTTCCGGTGT |
| | CAAGCAGACTCTGAACTTCGATCTGTTGAAGCTCGCGGGAGATGTCGAA |
| | AGCAACCCTGGTCCTCCTGCCGAGCTGGAGGTGGAATGTGCCACCCAGC |
| | TGCGCAGATTCGGGGACAAGCTCAACTTTCGGCAGAAGCTTCTCGGAAG |
| | CGGTGTGAAACAGACCCTGAATTTCGACCTCCTGAAACTTGCAGGAGAT |
| | GTGGAGTCAAACCCCGGACCGAACCTGTGGGCGGCGCAGAGATATGGA |
| | CGCGAACTGCGCCGGATGTCAGACGAGTTCGTCGACAGCTTCAAGAAAG |
| | GGGGTTCCGGAGTAAAGCAGACGCTGAACTTTGATCTGCTTAAGCTGGC |
| | TGGCGACGTGGAAAGCAATCCGGGCCCGCCGGCTGAGCTCGAAGTGGA |
| | GTGCGCAACCCAACTTCGGCGGTTCGGCGACAAGCTGAACTTCAGACAG |
| | AAGCTGTTGGGCAGCGGAGTGAAGCAGACCTTGAACTTTGACCTTCTCA |
| | AGCTGGCCGGGGATGTGGAATCCAACCCTGGGCCCAATCTGTGGGCTGC |
| | CCAGCGCTACGGAAGAGAACTGCGGCGGATGTCGGACGAATTCGTGGAC |
| | TCCTTCAAAAAGGGCGGCAGCGGCGTGAAGCAAACCCTCAATTTCGATC |
| | TGCTGAAGTTGGCCGGGGAC |
| | ((Noxa-F2A-BadBH3-F2A)\hat{ }3_cV5) |
| 256 | ATGGAGGAGCAGTGGGCCAGAGAGATCGGCGCCCAGCTGAGAAGAATGG |
| | CCGACGACCTGAACGCCCAGTACGAGAGAAGAGGCAGCGGCGTGAAGCA |
| | GACCCTGAACTTCGACCTGCTGAAGCTGGCCGGCGACGTGGAGAGCAACC |
| | CCGGCCCCGAGGAGCAGTGGGCCAGAGAGATCGGCGCCCAGCTGAGAAG |
| | AATGGCCGACGACCTGAACGCCCAGTACGAGAGAAGAGGCAGCGGCGTG |
| | AAGCAGACCCTGAACTTCGACCTGCTGAAGCTGGCCGGCGACGTGGAGA |
| | GCAACCCCGGCCCCGAGGAGCAGTGGGCCAGAGAGATCGGCGCCCAGCT |
| | GAGAAGAATGGCCGACGACCTGAACGCCCAGTACGAGAGAAGAGGCAGC |
| | GGCGTGAAGCAGACCCTGAACTTCGACCTGCTGAAGCTGGCCGGCGACGT |
| | GGAGAGCAACCCCGGCCCCGAGGAGCAGTGGGCCAGAGAGATCGGCGCC |
| | CAGCTGAGAAGAATGGCCGACGACCTGAACGCCCAGTACGAGAGAAGAG |
| | GCAGCGGCGTGAAGCAGACCCTGAACTTCGACCTGCTGAAGCTGGCCGGC |
| | GACGTGGAGAGCAACCCCGGCCCCGAGGAGCAGTGGGCCAGAGAGATCG |
| | GCGCCCAGCTGAGAAGAATGGCCGACGACCTGAACGCCCAGTACGAGAG |
| | AAGAGGCAGCGGCGTGAAGCAGACCCTGAACTTCGACCTGCTGAAGCTG |
| | GCCGGCGACGTGGAGAGCAACCCCGGCCCCGAGGAGCAGTGGGCCAGAG |
| | AGATCGGCGCCCAGCTGAGAAGAATGGCCGACGACCTGAACGCCCAGTA |
| | CGAGAGAAGAGGCAGCGGCGTGAAGCAGACCCTGAACTTCGACCTGCTG |
| | AAGCTGGCCGGCGACGTGGAGAGCAACCCCGGCCCCGAGGAGCAGTGGG |
| | CCAGAGAGATCGGCGCCCAGCTGAGAAGAATGGCCGACGACCTGAACGC |
| | CCAGTACGAGAGAAGAGGCAGCGGCGTGAAGCAGACCCTGAACTTCGAC |
| | CTGCTGAAGCTGGCCGGCGACGTGGAGAGCAACCCCGGCCCCGAGGAGC |
| | AGTGGGCCAGAGAGATCGGCGCCCAGCTGAGAAGAATGGCCGACGACCT |
| | GAACGCCCAGTACGAGAGAAGAGGCAGCGGCGTGAAGCAGACCCTGAAC |
| | TTCGACCTGCTGAAGCTGGCCGGCGACGTGGAGAGCAACCCCGGCCCCGA |
| | GGAGCAGTGGGCCAGAGAGATCGGCGCCCAGCTGAGAAGAATGGCCGAC |
| | GACCTGAACGCCCAGTACGAGAGAAGAGGCAGCGGCGTGAAGCAGACCC |
| | TGAACTTCGACCTGCTGAAGCTGGCCGGCGACGTGGAGAGCAACCCCGGC |
| | CCCGAGGAGCAGTGGGCCAGAGAGATCGGCGCCCAGCTGAGAAGAATGG |
| | CCGACGACCTGAACGCCCAGTACGAGAGAAGAGGCAGCGGCGTGAAGCA |
| | GACCCTGAACTTCGACCTGCTGAAGCTGGCCGGCGACGTGGAGAGCAACC |
| | CCGGCCCCGGCAAGCCCATCCCCAACCCCCTGCTGGGCCTGGACAGCACC |
| | ATGGAGGAGCAGTGGGCCAGAGAGATCGGCGCCCAGCTGAGAAGAATGG |
| | CCGACGACCTGAACGCCCAGTACGAGAGAAGAGGCAGCGGCGTGAAGCA |
| | GACCCTGAACTTCGACCTGCTGAAGCTGGCCGGCGACGTGGAGAGCAACC |
| | CCGGCCCCGAGGAGCAGTGGGCCAGAGAGATCGGCGCCCAGCTGAGAAG |
| | AATGGCCGACGACCTGAACGCCCAGTACGAGAGAAGAGGCAGCGGCGTG |
| | AAGCAGACCCTGAACTTCGACCTGCTGAAGCTGGCCGGCGACGTGGAGA |
| | GCAACCCCGGCCCCGAGGAGCAGTGGGCCAGAGAGATCGGCGCCCAGCT |
| | GAGAAGAATGGCCGACGACCTGAACGCCCAGTACGAGAGAAGAGGCAGC |
| | GGCGTGAAGCAGACCCTGAACTTCGACCTGCTGAAGCTGGCCGGCGACGT |
| | GGAGAGCAACCCCGGCCCCGAGGAGCAGTGGGCCAGAGAGATCGGCGCC |
| | CAGCTGAGAAGAATGGCCGACGACCTGAACGCCCAGTACGAGAGAAGAG |
| | GCAGCGGCGTGAAGCAGACCCTGAACTTCGACCTGCTGAAGCTGGCCGGC |
| | GACGTGGAGAGCAACCCCGGCCCCGAGGAGCAGTGGGCCAGAGAGATCG |
| | GCGCCCAGCTGAGAAGAATGGCCGACGACCTGAACGCCCAGTACGAGAG |
| | AAGAGGCAGCGGCGTGAAGCAGACCCTGAACTTCGACCTGCTGAAGCTG |
| | GCCGGCGACGTGGAGAGCAACCCCGGCCCCGAGGAGCAGTGGGCCAGAG |
| | AGATCGGCGCCCAGCTGAGAAGAATGGCCGACGACCTGAACGCCCAGTA |
| | CGAGAGAAGAGGCAGCGGCGTGAAGCAGACCCTGAACTTCGACCTGCTG |
| | AAGCTGGCCGGCGACGTGGAGAGCAACCCCGGCCCCGAGGAGCAGTGGG |
| | CCAGAGAGATCGGCGCCCAGCTGAGAAGAATGGCCGACGACCTGAACGC |
| | CCAGTACGAGAGAAGAGGCAGCGGCGTGAAGCAGACCCTGAACTTCGAC |
| | CTGCTGAAGCTGGCCGGCGACGTGGAGAGCAACCCCGGCCCCGAGGAGC |
| | AGTGGGCCAGAGAGATCGGCGCCCAGCTGAGAAGAATGGCCGACGACCT |
| | GAACGCCCAGTACGAGAGAAGAGGCAGCGGCGTGAAGCAGACCCTGAAC |
| | TTCGACCTGCTGAAGCTGGCCGGCGACGTGGAGAGCAACCCCGGCCCCGA |
| | GGAGCAGTGGGCCAGAGAGATCGGCGCCCAGCTGAGAAGAATGGCCGAC |
| | GACCTGAACGCCCAGTACGAGAGAAGAGGCAGCGGCGTGAAGCAGACCC |
| | TGAACTTCGACCTGCTGAAGCTGGCCGGCGACGTGGAGAGCAACCCCGGC |
| | CCCGAGGAGCAGTGGGCCAGAGAGATCGGCGCCCAGCTGAGAAGAATGG |
| | CCGACGACCTGAACGCCCAGTACGAGAGAAGAGGCAGCGGCGTGAAGCA |
| | GACCCTGAACTTCGACCTGCTGAAGCTGGCCGGCGACGTGGAGAGCAACC |
| | CCGGCCCCGGCAAGCCCATCCCCAACCCCCTGCTGGGCCTGGACAGCACC |
| | ((pumaBH3.F2A)\hat{ }10_cV5, (pumaBH3.F2A)\hat{ }10_cV5_DX) |
| 257 | ATGGACATGAGACCCGAGATCTGGATCGCCCAGGAGCTGAGAAGAATCG |
| | GCGACGAGTTCAACGCCTACTACGCCAGAAGAGGCAGCGGCGTGAAGCA |
| | GACCCTGAACTTCGACCTGCTGAAGCTGGCCGGCGACGTGGAGAGCAACC |
| | CCGGCCCCGACATGAGACCCGAGATCTGGATCGCCCAGGAGCTGAGAAG |
| | AATCGGCGACGAGTTCAACGCCTACTACGCCAGAAGAGGCAGCGGCGTG |
| | AAGCAGACCCTGAACTTCGACCTGCTGAAGCTGGCCGGCGACGTGGAGA |
| | GCAACCCCGGCCCCGACATGAGACCCGAGATCTGGATCGCCCAGGAGCTG |
| | AGAAGAATCGGCGACGAGTTCAACGCCTACTACGCCAGAAGAGGCAGCG |
| | GCGTGAAGCAGACCCTGAACTTCGACCTGCTGAAGCTGGCCGGCGACGTG |
| | GAGAGCAACCCCGGCCCCGACATGAGACCCGAGATCTGGATCGCCCAGG |
| | AGCTGAGAAGAATCGGCGACGAGTTCAACGCCTACTACGCCAGAAGAGG |
| | CAGCGGCGTGAAGCAGACCCTGAACTTCGACCTGCTGAAGCTGGCCGGCG |
| | ACGTGGAGAGCAACCCCGGCCCCGACATGAGACCCGAGATCTGGATCGC |
| | CCAGGAGCTGAGAAGAATCGGCGACGAGTTCAACGCCTACTACGCCAGA |
| | AGAGGCAGCGGCGTGAAGCAGACCCTGAACTTCGACCTGCTGAAGCTGG |
| | CCGGCGACGTGGAGAGCAACCCCGGCCCCGACATGAGACCCGAGATCTG |
| | GATCGCCCAGGAGCTGAGAAGAATCGGCGACGAGTTCAACGCCTACTAC |
| | GCCAGAAGAGGCAGCGGCGTGAAGCAGACCCTGAACTTCGACCTGCTGA |
| | AGCTGGCCGGCGACGTGGAGAGCAACCCCGGCCCCGACATGAGACCCGA |
| | GATCTGGATCGCCCAGGAGCTGAGAAGAATCGGCGACGAGTTCAACGCCT |
| | ACTACGCCAGAAGAGGCAGCGGCGTGAAGCAGACCCTGAACTTCGACCT |
| | GCTGAAGCTGGCCGGCGACGTGGAGAGCAACCCCGGCCCCGACATGAGA |
| | CCCGAGATCTGGATCGCCCAGGAGCTGAGAAGAATCGGCGACGAGTTCA |
| | ACGCCTACTACGCCAGAAGAGGCAGCGGCGTGAAGCAGACCCTGAACTT |
| | CGACCTGCTGAAGCTGGCCGGCGACGTGGAGAGCAACCCCGGCCCCGAC |
| | ATGAGACCCGAGATCTGGATCGCCCAGGAGCTGAGAAGAATCGGCGACG |
| | AGTTCAACGCCTACTACGCCAGAAGAGGCAGCGGCGTGAAGCAGACCCT |
| | GAACTTCGACCTGCTGAAGCTGGCCGGCGACGTGGAGAGCAACCCCGGCC |
| | CCGACATGAGACCCGAGATCTGGATCGCCCAGGAGCTGAGAAGAATCGG |
| | CGACGAGTTCAACGCCTACTACGCCAGAAGAGGCAGCGGCGTGAAGCAG |
| | ACCCTGAACTTCGACCTGCTGAAGCTGGCCGGCGACGTGGAGAGCAACCC |
| | CGGCCCCGGCAAGCCCATCCCCAACCCCCTGCTGGGCCTGGACAGCACCA |
| | TGGACATGAGACCCGAGATCTGGATCGCCCAGGAGCTGAGAAGAATCGG |
| | CGACGAGTTCAACGCCTACTACGCCAGAAGAGGCAGCGGCGTGAAGCAG |
| | ACCCTGAACTTCGACCTGCTGAAGCTGGCCGGCGACGTGGAGAGCAACCC |
| | CGGCCCCGACATGAGACCCGAGATCTGGATCGCCCAGGAGCTGAGAAGA |
| | ATCGGCGACGAGTTCAACGCCTACTACGCCAGAAGAGGCAGCGGCGTGA |
| | AGCAGACCCTGAACTTCGACCTGCTGAAGCTGGCCGGCGACGTGGAGAGC |
| | AACCCCGGCCCCGACATGAGACCCGAGATCTGGATCGCCCAGGAGCTGA |
| | GAAGAATCGGCGACGAGTTCAACGCCTACTACGCCAGAAGAGGCAGCGG |
| | CGTGAAGCAGACCCTGAACTTCGACCTGCTGAAGCTGGCCGGCGACGTGG |
| | AGAGCAACCCCGGCCCCGACATGAGACCCGAGATCTGGATCGCCCAGGA |
| | GCTGAGAAGAATCGGCGACGAGTTCAACGCCTACTACGCCAGAAGAGGC |
| | AGCGGCGTGAAGCAGACCCTGAACTTCGACCTGCTGAAGCTGGCCGGCGA |
| | CGTGGAGAGCAACCCCGGCCCCGACATGAGACCCGAGATCTGGATCGCCC |
| | AGGAGCTGAGAAGAATCGGCGACGAGTTCAACGCCTACTACGCCAGAAG |
| | AGGCAGCGGCGTGAAGCAGACCCTGAACTTCGACCTGCTGAAGCTGGCCG |
| | GCGACGTGGAGAGCAACCCCGGCCCCGACATGAGACCCGAGATCTGGAT |
| | CGCCCAGGAGCTGAGAAGAATCGGCGACGAGTTCAACGCCTACTACGCC |
| | AGAAGAGGCAGCGGCGTGAAGCAGACCCTGAACTTCGACCTGCTGAAGC |
| | TGGCCGGCGACGTGGAGAGCAACCCCGGCCCCGACATGAGACCCGAGAT |
| | CTGGATCGCCCAGGAGCTGAGAAGAATCGGCGACGAGTTCAACGCCTACT |
| | ACGCCAGAAGAGGCAGCGGCGTGAAGCAGACCCTGAACTTCGACCTGCT |
| | GAAGCTGGCCGGCGACGTGGAGAGCAACCCCGGCCCCGACATGAGACCC |
| | GAGATCTGGATCGCCCAGGAGCTGAGAAGAATCGGCGACGAGTTCAACG |
| | CCTACTACGCCAGAAGAGGCAGCGGCGTGAAGCAGACCCTGAACTTCGA |
| | CCTGCTGAAGCTGGCCGGCGACGTGGAGAGCAACCCCGGCCCCGACATG |
| | AGACCCGAGATCTGGATCGCCCAGGAGCTGAGAAGAATCGGCGACGAGT |
| | TCAACGCCTACTACGCCAGAAGAGGCAGCGGCGTGAAGCAGACCCTGAA |
| | CTTCGACCTGCTGAAGCTGGCCGGCGACGTGGAGAGCAACCCCGGCCCCG |
| | ACATGAGACCCGAGATCTGGATCGCCCAGGAGCTGAGAAGAATCGGCGA |
| | CGAGTTCAACGCCTACTACGCCAGAAGAGGCAGCGGCGTGAAGCAGACC |
| | CTGAACTTCGACCTGCTGAAGCTGGCCGGCGACGTGGAGAGCAACCCCGG |
| | CCCCGGCAAGCCCATCCCCAACCCCCTGCTGGGCCTGGACAGCACC |
| | ((BimBH3.F2A)\hat{ }10_cV5, (BimBH3.F2A)\hat{ }10_cV5_DX) |
| 258 | ATGGAGGAGCAGTGGGCCAGAGAGATCGGCGCCCAGCTGAGAAGAATGG |
| | CCGACGACCTGAACGCCCAGTACGAGAGAAGAGGCAGCGGCGTGAAGCA |
| | GACCCTGAACTTCGACCTGCTGAAGCTGGCCGGCGACGTGGAGAGCAACC |
| | CCGGCCCCGACATGAGACCCGAGATCTGGATCGCCCAGGAGCTGAGAAG |
| | AATCGGCGACGAGTTCAACGCCTACTACGCCAGAAGAGGCAGCGGCGTG |
| | AAGCAGACCCTGAACTTCGACCTGCTGAAGCTGGCCGGCGACGTGGAGA |
| | GCAACCCCGGCCCCGAGGAGCAGTGGGCCAGAGAGATCGGCGCCCAGCT |
| | GAGAAGAATGGCCGACGACCTGAACGCCCAGTACGAGAGAAGAGGCAGC |
| | GGCGTGAAGCAGACCCTGAACTTCGACCTGCTGAAGCTGGCCGGCGACGT |
| | GGAGAGCAACCCCGGCCCCGACATGAGACCCGAGATCTGGATCGCCCAG |
| | GAGCTGAGAAGAATCGGCGACGAGTTCAACGCCTACTACGCCAGAAGAG |
| | GCAGCGGCGTGAAGCAGACCCTGAACTTCGACCTGCTGAAGCTGGCCGGC |
| | GACGTGGAGAGCAACCCCGGCCCCGAGGAGCAGTGGGCCAGAGAGATCG |
| | GCGCCCAGCTGAGAAGAATGGCCGACGACCTGAACGCCCAGTACGAGAG |
| | AAGAGGCAGCGGCGTGAAGCAGACCCTGAACTTCGACCTGCTGAAGCTG |
| | GCCGGCGACGTGGAGAGCAACCCCGGCCCCGACATGAGACCCGAGATCT |
| | GGATCGCCCAGGAGCTGAGAAGAATCGGCGACGAGTTCAACGCCTACTA |
| | CGCCAGAAGAGGCAGCGGCGTGAAGCAGACCCTGAACTTCGACCTGCTG |
| | AAGCTGGCCGGCGACGTGGAGAGCAACCCCGGCCCCGGCAAGCCCATCC |
| | CCAACCCCCTGCTGGGCCTGGACAGCACC |
| | ((Puma-P2A-BimBH3-F2A)\hat{ }3_cV5) |
| 259 | ATGGAGGAGCAGTGGGCCAGAGAGATCGGCGCCCAGCTGAGAAGAATGG |
| | CCGACGACCTGAACGCCCAGTACGAGAGAAGAGGCAGCGGCGTGAAGCA |
| | GACCCTGAACTTCGACCTGCTGAAGCTGGCCGGCGACGTGGAGAGCAACC |
| | CCGGCCCCGACATGAGACCCGAGATCTGGATCGCCCAGGAGCTGAGAAG |
| | AATCGGCGACGAGTTCAACGCCTACTACGCCAGAAGAGGCAGCGGCGTG |
| | AAGCAGACCCTGAACTTCGACCTGCTGAAGCTGGCCGGCGACGTGGAGA |
| | GCAACCCCGGCCCCGAGGAGCAGTGGGCCAGAGAGATCGGCGCCCAGCT |
| | GAGAAGAATGGCCGACGACCTGAACGCCCAGTACGAGAGAAGAGGCAGC |
| | GGCGTGAAGCAGACCCTGAACTTCGACCTGCTGAAGCTGGCCGGCGACGT |
| | GGAGAGCAACCCCGGCCCCGACATGAGACCCGAGATCTGGATCGCCCAG |
| | GAGCTGAGAAGAATCGGCGACGAGTTCAACGCCTACTACGCCAGAAGAG |
| | GCAGCGGCGTGAAGCAGACCCTGAACTTCGACCTGCTGAAGCTGGCCGGC |
| | GACGTGGAGAGCAACCCCGGCCCCGAGGAGCAGTGGGCCAGAGAGATCG |
| | GCGCCCAGCTGAGAAGAATGGCCGACGACCTGAACGCCCAGTACGAGAG |
| | AAGAGGCAGCGGCGTGAAGCAGACCCTGAACTTCGACCTGCTGAAGCTG |
| | GCCGGCGACGTGGAGAGCAACCCCGGCCCCGACATGAGACCCGAGATCT |
| | GGATCGCCCAGGAGCTGAGAAGAATCGGCGACGAGTTCAACGCCTACTA |
| | CGCCAGAAGAGGCAGCGGCGTGAAGCAGACCCTGAACTTCGACCTGCTG |
| | AAGCTGGCCGGCGACGTGGAGAGCAACCCCGGCCCCGAGGAGCAGTGGG |
| | CCAGAGAGATCGGCGCCCAGCTGAGAAGAATGGCCGACGACCTGAACGC |
| | CCAGTACGAGAGAAGAGGCAGCGGCGTGAAGCAGACCCTGAACTTCGAC |
| | CTGCTGAAGCTGGCCGGCGACGTGGAGAGCAACCCCGGCCCCGACATGA |
| | GACCCGAGATCTGGATCGCCCAGGAGCTGAGAAGAATCGGCGACGAGTT |
| | CAACGCCTACTACGCCAGAAGAGGCAGCGGCGTGAAGCAGACCCTGAAC |
| | TTCGACCTGCTGAAGCTGGCCGGCGACGTGGAGAGCAACCCCGGCCCCGA |
| | GGAGCAGTGGGCCAGAGAGATCGGCGCCCAGCTGAGAAGAATGGCCGAC |
| | GACCTGAACGCCCAGTACGAGAGAAGAGGCAGCGGCGTGAAGCAGACCC |
| | TGAACTTCGACCTGCTGAAGCTGGCCGGCGACGTGGAGAGCAACCCCGGC |
| | CCCGACATGAGACCCGAGATCTGGATCGCCCAGGAGCTGAGAAGAATCG |
| | GCGACGAGTTCAACGCCTACTACGCCAGAAGAGGCAGCGGCGTGAAGCA |
| | GACCCTGAACTTCGACCTGCTGAAGCTGGCCGGCGACGTGGAGAGCAACC |
| | CCGGCCCCGGCAAGCCCATCCCCAACCCCCTGCTGGGCCTGGACAGCACC |
| | ((Puma-F2A-BimBH3-F2A)\hat{ }5_cV5, (Puma-F2A-BimBH3F2A)\hat{ }5_cV5_DX) |
| 260 | ATGGAGGAGCAGTGGGCCAGAGAGATCGGCGCCCAGCTGAGAAGAATGG |
| | CCGACGACCTGAACGCCCAGTACGAGAGAAGAGGCAGCGGCGTGAAGCA |
| | GACCCTGAACTTCGACCTGCTGAAGCTGGCCGGCGACGTGGAGAGCAACC |
| | CCGGCCCCGACATGAGACCCGAGATCTGGATCGCCCAGGAGCTGAGAAG |
| | AATCGGCGACGAGTTCAACGCCTACTACGCCAGAAGAGGCAGCGGCGTG |
| | AAGCAGACCCTGAACTTCGACCTGCTGAAGCTGGCCGGCGACGTGGAGA |
| | GCAACCCCGGCCCCGAGGAGCAGTGGGCCAGAGAGATCGGCGCCCAGCT |
| | GAGAAGAATGGCCGACGACCTGAACGCCCAGTACGAGAGAAGAGGCAGC |
| | GGCGTGAAGCAGACCCTGAACTTCGACCTGCTGAAGCTGGCCGGCGACGT |
| | GGAGAGCAACCCCGGCCCCGACATGAGACCCGAGATCTGGATCGCCCAG |
| | GAGCTGAGAAGAATCGGCGACGAGTTCAACGCCTACTACGCCAGAAGAG |
| | GCAGCGGCGTGAAGCAGACCCTGAACTTCGACCTGCTGAAGCTGGCCGGC |
| | GACGTGGAGAGCAACCCCGGCCCCGAGGAGCAGTGGGCCAGAGAGATCG |
| | GCGCCCAGCTGAGAAGAATGGCCGACGACCTGAACGCCCAGTACGAGAG |
| | AAGAGGCAGCGGCGTGAAGCAGACCCTGAACTTCGACCTGCTGAAGCTG |
| | GCCGGCGACGTGGAGAGCAACCCCGGCCCCGACATGAGACCCGAGATCT |
| | GGATCGCCCAGGAGCTGAGAAGAATCGGCGACGAGTTCAACGCCTACTA |
| | CGCCAGAAGAGGCAGCGGCGTGAAGCAGACCCTGAACTTCGACCTGCTG |
| | AAGCTGGCCGGCGACGTGGAGAGCAACCCCGGCCCCGAGGAGCAGTGGG |
| | CCAGAGAGATCGGCGCCCAGCTGAGAAGAATGGCCGACGACCTGAACGC |
| | CCAGTACGAGAGAAGAGGCAGCGGCGTGAAGCAGACCCTGAACTTCGAC |
| | CTGCTGAAGCTGGCCGGCGACGTGGAGAGCAACCCCGGCCCCGACATGA |
| | GACCCGAGATCTGGATCGCCCAGGAGCTGAGAAGAATCGGCGACGAGTT |
| | CAACGCCTACTACGCCAGAAGAGGCAGCGGCGTGAAGCAGACCCTGAAC |
| | TTCGACCTGCTGAAGCTGGCCGGCGACGTGGAGAGCAACCCCGGCCCCGA |
| | GGAGCAGTGGGCCAGAGAGATCGGCGCCCAGCTGAGAAGAATGGCCGAC |
| | GACCTGAACGCCCAGTACGAGAGAAGAGGCAGCGGCGTGAAGCAGACCC |
| | TGAACTTCGACCTGCTGAAGCTGGCCGGCGACGTGGAGAGCAACCCCGGC |
| | CCCGACATGAGACCCGAGATCTGGATCGCCCAGGAGCTGAGAAGAATCG |
| | GCGACGAGTTCAACGCCTACTACGCCAGAAGAGGCAGCGGCGTGAAGCA |
| | GACCCTGAACTTCGACCTGCTGAAGCTGGCCGGCGACGTGGAGAGCAACC |
| | CCGGCCCCGAGGAGCAGTGGGCCAGAGAGATCGGCGCCCAGCTGAGAAG |
| | AATGGCCGACGACCTGAACGCCCAGTACGAGAGAAGAGGCAGCGGCGTG |
| | AAGCAGACCCTGAACTTCGACCTGCTGAAGCTGGCCGGCGACGTGGAGA |
| | GCAACCCCGGCCCCGACATGAGACCCGAGATCTGGATCGCCCAGGAGCTG |
| | AGAAGAATCGGCGACGAGTTCAACGCCTACTACGCCAGAAGAGGCAGCG |
| | GCGTGAAGCAGACCCTGAACTTCGACCTGCTGAAGCTGGCCGGCGACGTG |
| | GAGAGCAACCCCGGCCCCGAGGAGCAGTGGGCCAGAGAGATCGGCGCCC |
| | AGCTGAGAAGAATGGCCGACGACCTGAACGCCCAGTACGAGAGAAGAGG |
| | CAGCGGCGTGAAGCAGACCCTGAACTTCGACCTGCTGAAGCTGGCCGGCG |
| | ACGTGGAGAGCAACCCCGGCCCCGACATGAGACCCGAGATCTGGATCGC |
| | CCAGGAGCTGAGAAGAATCGGCGACGAGTTCAACGCCTACTACGCCAGA |
| | AGAGGCAGCGGCGTGAAGCAGACCCTGAACTTCGACCTGCTGAAGCTGG |
| | CCGGCGACGTGGAGAGCAACCCCGGCCCCGAGGAGCAGTGGGCCAGAGA |
| | GATCGGCGCCCAGCTGAGAAGAATGGCCGACGACCTGAACGCCCAGTAC |
| | GAGAGAAGAGGCAGCGGCGTGAAGCAGACCCTGAACTTCGACCTGCTGA |
| | AGCTGGCCGGCGACGTGGAGAGCAACCCCGGCCCCGACATGAGACCCGA |
| | GATCTGGATCGCCCAGGAGCTGAGAAGAATCGGCGACGAGTTCAACGCCT |
| | ACTACGCCAGAAGAGGCAGCGGCGTGAAGCAGACCCTGAACTTCGACCT |
| | GCTGAAGCTGGCCGGCGACGTGGAGAGCAACCCCGGCCCCGAGGAGCAG |
| | TGGGCCAGAGAGATCGGCGCCCAGCTGAGAAGAATGGCCGACGACCTGA |
| | ACGCCCAGTACGAGAGAAGAGGCAGCGGCGTGAAGCAGACCCTGAACTT |
| | CGACCTGCTGAAGCTGGCCGGCGACGTGGAGAGCAACCCCGGCCCCGAC |
| | ATGAGACCCGAGATCTGGATCGCCCAGGAGCTGAGAAGAATCGGCGACG |
| | AGTTCAACGCCTACTACGCCAGAAGAGGCAGCGGCGTGAAGCAGACCCT |
| | GAACTTCGACCTGCTGAAGCTGGCCGGCGACGTGGAGAGCAACCCCGGCC |
| | CCGAGGAGCAGTGGGCCAGAGAGATCGGCGCCCAGCTGAGAAGAATGGC |
| | CGACGACCTGAACGCCCAGTACGAGAGAAGAGGCAGCGGCGTGAAGCAG |
| | ACCCTGAACTTCGACCTGCTGAAGCTGGCCGGCGACGTGGAGAGCAACCC |
| | CGGCCCCGACATGAGACCCGAGATCTGGATCGCCCAGGAGCTGAGAAGA |
| | ATCGGCGACGAGTTCAACGCCTACTACGCCAGAAGAGGCAGCGGCGTGA |
| | AGCAGACCCTGAACTTCGACCTGCTGAAGCTGGCCGGCGACGTGGAGAGC |
| | AACCCCGGCCCCGGCAAGCCCATCCCCAACCCCCTGCTGGGCCTGGACAG |
| | CACC |
| | ((Puma-F2A-BimBH3-F2A)\hat{ }10_cV5) |
| 261 | ATGCAGTGGGCCAGAGAGATCGGCGCCCAGCTGAGAAGAATCGGCGACG |
| | ACCTGAACGCCCAGTACGAGAGAAGAAGACAGGGCAGCGGCGTGAAGCA |
| | GACCCTGAACTTCGACCTGCTGAAGCTGGCCGGCGACGTGGAGAGCAACC |
| | CCGGCCCCCAGTGGGCCAGAGAGATCGGCGCCCAGCTGAGAAGAATCGG |
| | CGACGACCTGAACGCCCAGTACGAGAGAAGAAGACAGGGCAGCGGCGTG |
| | AAGCAGACCCTGAACTTCGACCTGCTGAAGCTGGCCGGCGACGTGGAGA |
| | GCAACCCCGGCCCCCAGTGGGCCAGAGAGATCGGCGCCCAGCTGAGAAG |
| | AATCGGCGACGACCTGAACGCCCAGTACGAGAGAAGAAGACAGGGCAGC |
| | GGCGTGAAGCAGACCCTGAACTTCGACCTGCTGAAGCTGGCCGGCGACGT |
| | GGAGAGCAACCCCGGCCCCCAGTGGGCCAGAGAGATCGGCGCCCAGCTG |
| | AGAAGAATCGGCGACGACCTGAACGCCCAGTACGAGAGAAGAAGACAGG |
| | GCAGCGGCGTGAAGCAGACCCTGAACTTCGACCTGCTGAAGCTGGCCGGC |
| | GACGTGGAGAGCAACCCCGGCCCCCAGTGGGCCAGAGAGATCGGCGCCC |
| | AGCTGAGAAGAATCGGCGACGACCTGAACGCCCAGTACGAGAGAAGAAG |
| | ACAGGGCAGCGGCGTGAAGCAGACCCTGAACTTCGACCTGCTGAAGCTG |
| | GCCGGCGACGTGGAGAGCAACCCCGGCCCCCAGTGGGCCAGAGAGATCG |
| | GCGCCCAGCTGAGAAGAATCGGCGACGACCTGAACGCCCAGTACGAGAG |
| | AAGAAGACAGGGCAGCGGCGTGAAGCAGACCCTGAACTTCGACCTGCTG |
| | AAGCTGGCCGGCGACGTGGAGAGCAACCCCGGCCCCCAGTGGGCCAGAG |
| | AGATCGGCGCCCAGCTGAGAAGAATCGGCGACGACCTGAACGCCCAGTA |
| | CGAGAGAAGAAGACAGGGCAGCGGCGTGAAGCAGACCCTGAACTTCGAC |
| | CTGCTGAAGCTGGCCGGCGACGTGGAGAGCAACCCCGGCCCCCAGTGGG |
| | CCAGAGAGATCGGCGCCCAGCTGAGAAGAATCGGCGACGACCTGAACGC |
| | CCAGTACGAGAGAAGAAGACAGGGCAGCGGCGTGAAGCAGACCCTGAAC |
| | TTCGACCTGCTGAAGCTGGCCGGCGACGTGGAGAGCAACCCCGGCCCCCA |
| | GTGGGCCAGAGAGATCGGCGCCCAGCTGAGAAGAATCGGCGACGACCTG |
| | AACGCCCAGTACGAGAGAAGAAGACAGGGCAGCGGCGTGAAGCAGACCC |
| | TGAACTTCGACCTGCTGAAGCTGGCCGGCGACGTGGAGAGCAACCCCGGC |
| | CCCCAGTGGGCCAGAGAGATCGGCGCCCAGCTGAGAAGAATCGGCGACG |
| | ACCTGAACGCCCAGTACGAGAGAAGAAGACAGGGCAGCGGCGTGAAGCA |
| | GACCCTGAACTTCGACCTGCTGAAGCTGGCCGGCGACGTGGAGAGCAACC |
| | CCGGCCCCGGCAAGCCCATCCCCAACCCCCTGCTGGGCCTGGACAGCACC |
| | ((SuperPumaBH3.F2A)\hat{ }10_cV5, (SuperPumaBH3.F2A)\hat{ }10_cV5_DX) |
| 262 | ATGCAGTGGGCCAGAGAGATCGGCGCCCAGGAGAGAAGAGAGGCCGACG |
| | ACGAGAACGCCCAGTACGAGAGAAGAAGACAGGGCAGCGGCGTGAAGC |
| | AGACCCTGAACTTCGACCTGCTGAAGCTGGCCGGCGACGTGGAGAGCAAC |
| | CCCGGCCCCCAGTGGGCCAGAGAGATCGGCGCCCAGGAGAGAAGAGAGG |
| | CCGACGACGAGAACGCCCAGTACGAGAGAAGAAGACAGGGCAGCGGCGT |
| | GAAGCAGACCCTGAACTTCGACCTGCTGAAGCTGGCCGGCGACGTGGAG |
| | AGCAACCCCGGCCCCCAGTGGGCCAGAGAGATCGGCGCCCAGGAGAGAA |
| | GAGAGGCCGACGACGAGAACGCCCAGTACGAGAGAAGAAGACAGGGCA |
| | GCGGCGTGAAGCAGACCCTGAACTTCGACCTGCTGAAGCTGGCCGGCGAC |
| | GTGGAGAGCAACCCCGGCCCCCAGTGGGCCAGAGAGATCGGCGCCCAGG |
| | AGAGAAGAGAGGCCGACGACGAGAACGCCCAGTACGAGAGAAGAAGAC |
| | AGGGCAGCGGCGTGAAGCAGACCCTGAACTTCGACCTGCTGAAGCTGGCC |
| | GGCGACGTGGAGAGCAACCCCGGCCCCCAGTGGGCCAGAGAGATCGGCG |
| | CCCAGGAGAGAAGAGAGGCCGACGACGAGAACGCCCAGTACGAGAGAA |
| | GAAGACAGGGCAGCGGCGTGAAGCAGACCCTGAACTTCGACCTGCTGAA |
| | GCTGGCCGGCGACGTGGAGAGCAACCCCGGCCCCCAGTGGGCCAGAGAG |
| | ATCGGCGCCCAGGAGAGAAGAGAGGCCGACGACGAGAACGCCCAGTACG |
| | AGAGAAGAAGACAGGGCAGCGGCGTGAAGCAGACCCTGAACTTCGACCT |
| | GCTGAAGCTGGCCGGCGACGTGGAGAGCAACCCCGGCCCCCAGTGGGCC |
| | AGAGAGATCGGCGCCCAGGAGAGAAGAGAGGCCGACGACGAGAACGCC |
| | CAGTACGAGAGAAGAAGACAGGGCAGCGGCGTGAAGCAGACCCTGAACT |
| | TCGACCTGCTGAAGCTGGCCGGCGACGTGGAGAGCAACCCCGGCCCCCAG |
| | TGGGCCAGAGAGATCGGCGCCCAGGAGAGAAGAGAGGCCGACGACGAG |
| | AACGCCCAGTACGAGAGAAGAAGACAGGGCAGCGGCGTGAAGCAGACCC |
| | TGAACTTCGACCTGCTGAAGCTGGCCGGCGACGTGGAGAGCAACCCCGGC |
| | CCCCAGTGGGCCAGAGAGATCGGCGCCCAGGAGAGAAGAGAGGCCGACG |
| | ACGAGAACGCCCAGTACGAGAGAAGAAGACAGGGCAGCGGCGTGAAGC |
| | AGACCCTGAACTTCGACCTGCTGAAGCTGGCCGGCGACGTGGAGAGCAAC |
| | CCCGGCCCCCAGTGGGCCAGAGAGATCGGCGCCCAGGAGAGAAGAGAGG |
| | CCGACGACGAGAACGCCCAGTACGAGAGAAGAAGACAGGGCAGCGGCGT |
| | GAAGCAGACCCTGAACTTCGACCTGCTGAAGCTGGCCGGCGACGTGGAG |
| | AGCAACCCCGGCCCCCAGTGGGCCAGAGAGATCGGCGCCCAGGAGAGAA |
| | GAGAGGCCGACGACGAGAACGCCCAGTACGAGAGAAGAAGACAGGGCA |
| | GCGGCGTGAAGCAGACCCTGAACTTCGACCTGCTGAAGCTGGCCGGCGAC |
| | GTGGAGAGCAACCCCGGCCCCGGCAAGCCCATCCCCAACCCCCTGCTGGG |
| | CCTGGACAGCACC |
| | ((KittyCatBH3.F2A)\hat{ }10_cV5, (KittyCatBH3.F2A)\hat{ }10_cV5_DX) |
| 263 | ATGCAGTGGGCCAGAGAGATCGGCGCCCAGGAGAGAAGAGAGGCCGACG |
| | ACGAGAACGCCCAGTACGAGAGAAGAAGACAGGGCAGCGGCGTGAAGC |
| | AGACCCTGAACTTCGACCTGCTGAAGCTGGCCGGCGACGTGGAGAGCAAC |
| | CCCGGCCCCCAGTGGGCCAGAGAGATCGGCGCCCAGGAGAGAAGAGAGG |
| | CCGACGACGAGAACGCCCAGTACGAGAGAAGAAGACAGGGCAGCGGCGT |
| | GAAGCAGACCCTGAACTTCGACCTGCTGAAGCTGGCCGGCGACGTGGAG |
| | AGCAACCCCGGCCCCCAGTGGGCCAGAGAGATCGGCGCCCAGGAGAGAA |
| | GAGAGGCCGACGACGAGAACGCCCAGTACGAGAGAAGAAGACAGGGCA |
| | GCGGCGTGAAGCAGACCCTGAACTTCGACCTGCTGAAGCTGGCCGGCGAC |
| | GTGGAGAGCAACCCCGGCCCCCAGTGGGCCAGAGAGATCGGCGCCCAGG |
| | AGAGAAGAGAGGCCGACGACGAGAACGCCCAGTACGAGAGAAGAAGAC |
| | AGGGCAGCGGCGTGAAGCAGACCCTGAACTTCGACCTGCTGAAGCTGGCC |
| | GGCGACGTGGAGAGCAACCCCGGCCCCCAGTGGGCCAGAGAGATCGGCG |
| | CCCAGGAGAGAAGAGAGGCCGACGACGAGAACGCCCAGTACGAGAGAA |
| | GAAGACAGGGCAGCGGCGTGAAGCAGACCCTGAACTTCGACCTGCTGAA |
| | GCTGGCCGGCGACGTGGAGAGCAACCCCGGCCCCCAGTGGGCCAGAGAG |
| | ATCGGCGCCCAGGAGAGAAGAGAGGCCGACGACGAGAACGCCCAGTACG |
| | AGAGAAGAAGACAGGGCAGCGGCGTGAAGCAGACCCTGAACTTCGACCT |
| | GCTGAAGCTGGCCGGCGACGTGGAGAGCAACCCCGGCCCCCAGTGGGCC |
| | AGAGAGATCGGCGCCCAGGAGAGAAGAGAGGCCGACGACGAGAACGCC |
| | CAGTACGAGAGAAGAAGACAGGGCAGCGGCGTGAAGCAGACCCTGAACT |
| | TCGACCTGCTGAAGCTGGCCGGCGACGTGGAGAGCAACCCCGGCCCCCAG |
| | TGGGCCAGAGAGATCGGCGCCCAGGAGAGAAGAGAGGCCGACGACGAG |
| | AACGCCCAGTACGAGAGAAGAAGACAGGGCAGCGGCGTGAAGCAGACCC |
| | TGAACTTCGACCTGCTGAAGCTGGCCGGCGACGTGGAGAGCAACCCCGGC |
| | CCCCAGTGGGCCAGAGAGATCGGCGCCCAGGAGAGAAGAGAGGCCGACG |
| | ACGAGAACGCCCAGTACGAGAGAAGAAGACAGGGCAGCGGCGTGAAGC |
| | AGACCCTGAACTTCGACCTGCTGAAGCTGGCCGGCGACGTGGAGAGCAAC |
| | CCCGGCCCCCAGTGGGCCAGAGAGATCGGCGCCCAGGAGAGAAGAGAGG |
| | CCGACGACGAGAACGCCCAGTACGAGAGAAGAAGACAGGGCAGCGGCGT |
| | GAAGCAGACCCTGAACTTCGACCTGCTGAAGCTGGCCGGCGACGTGGAG |
| | AGCAACCCCGGCCCCCAGTGGGCCAGAGAGATCGGCGCCCAGGAGAGAA |
| | GAGAGGCCGACGACGAGAACGCCCAGTACGAGAGAAGAAGACAGGGCA |
| | GCGGCGTGAAGCAGACCCTGAACTTCGACCTGCTGAAGCTGGCCGGCGAC |
| | GTGGAGAGCAACCCCGGCCCCGGCAAGCCCATCCCCAACCCCCTGCTGGG |
| | CCTGGACAGCACC |
| | ((KittyCatBH3.F2A)\hat{ }10_cV5, (KittyCatBH3.F2A)\hat{ }10_cV5_DX) |
| 264 | ATGCCCGCCGAGCTGGAGGTGGAGTGCGCCACCCAGCTGAGAAGATTCG |
| | GCGACAAGCTGAACTTCAGACAGAAGCTGCTGGGCAGCGGCGTGAAGCA |
| | GACCCTGAACTTCGACCTGCTGAAGCTGGCCGGCGACGTGGAGAGCAACC |
| | CCGGCCCCCCCGCCGAGCTGGAGGTGGAGTGCGCCACCCAGCTGAGAAG |
| | ATTCGGCGACAAGCTGAACTTCAGACAGAAGCTGCTGGGCAGCGGCGTG |
| | AAGCAGACCCTGAACTTCGACCTGCTGAAGCTGGCCGGCGACGTGGAGA |
| | GCAACCCCGGCCCCCCCGCCGAGCTGGAGGTGGAGTGCGCCACCCAGCTG |
| | AGAAGATTCGGCGACAAGCTGAACTTCAGACAGAAGCTGCTGGGCAGCG |
| | GCGTGAAGCAGACCCTGAACTTCGACCTGCTGAAGCTGGCCGGCGACGTG |
| | GAGAGCAACCCCGGCCCCCCCGCCGAGCTGGAGGTGGAGTGCGCCACCC |
| | AGCTGAGAAGATTCGGCGACAAGCTGAACTTCAGACAGAAGCTGCTGGG |
| | CAGCGGCGTGAAGCAGACCCTGAACTTCGACCTGCTGAAGCTGGCCGGCG |
| | ACGTGGAGAGCAACCCCGGCCCCCCCGCCGAGCTGGAGGTGGAGTGCGC |
| | CACCCAGCTGAGAAGATTCGGCGACAAGCTGAACTTCAGACAGAAGCTG |
| | CTGGGCAGCGGCGTGAAGCAGACCCTGAACTTCGACCTGCTGAAGCTGGC |
| | CGGCGACGTGGAGAGCAACCCCGGCCCCCCCGCCGAGCTGGAGGTGGAG |
| | TGCGCCACCCAGCTGAGAAGATTCGGCGACAAGCTGAACTTCAGACAGA |
| | AGCTGCTGGGCAGCGGCGTGAAGCAGACCCTGAACTTCGACCTGCTGAAG |
| | CTGGCCGGCGACGTGGAGAGCAACCCCGGCCCCGGCAAGCCCATCCCCA |
| | ACCCCCTGCTGGGCCTGGACAGCACC |
| | ((NoxaBH3.F2A)\hat{ }5_cV5, (NoxaBH3.F2A)\hat{ }5_cV5_DX) |
| 265 | ATGCCCGCCGAGCTGGAGGTGGAGTGCGCCACCCAGCTGAGAAGATTCG |
| | GCGACAAGCTGAACTTCAGACAGAAGCTGCTGGGCAGCGGCGTGAAGCA |
| | GACCCTGAACTTCGACCTGCTGAAGCTGGCCGGCGACGTGGAGAGCAACC |
| | CCGGCCCCCCCGCCGAGCTGGAGGTGGAGTGCGCCACCCAGCTGAGAAG |
| | ATTCGGCGACAAGCTGAACTTCAGACAGAAGCTGCTGGGCAGCGGCGTG |
| | AAGCAGACCCTGAACTTCGACCTGCTGAAGCTGGCCGGCGACGTGGAGA |
| | GCAACCCCGGCCCCCCCGCCGAGCTGGAGGTGGAGTGCGCCACCCAGCTG |
| | AGAAGATTCGGCGACAAGCTGAACTTCAGACAGAAGCTGCTGGGCAGCG |
| | GCGTGAAGCAGACCCTGAACTTCGACCTGCTGAAGCTGGCCGGCGACGTG |
| | GAGAGCAACCCCGGCCCCCCCGCCGAGCTGGAGGTGGAGTGCGCCACCC |
| | AGCTGAGAAGATTCGGCGACAAGCTGAACTTCAGACAGAAGCTGCTGGG |
| | CAGCGGCGTGAAGCAGACCCTGAACTTCGACCTGCTGAAGCTGGCCGGCG |
| | ACGTGGAGAGCAACCCCGGCCCCCCCGCCGAGCTGGAGGTGGAGTGCGC |
| | CACCCAGCTGAGAAGATTCGGCGACAAGCTGAACTTCAGACAGAAGCTG |
| | CTGGGCAGCGGCGTGAAGCAGACCCTGAACTTCGACCTGCTGAAGCTGGC |
| | CGGCGACGTGGAGAGCAACCCCGGCCCCCCCGCCGAGCTGGAGGTGGAG |
| | TGCGCCACCCAGCTGAGAAGATTCGGCGACAAGCTGAACTTCAGACAGA |
| | AGCTGCTGGGCAGCGGCGTGAAGCAGACCCTGAACTTCGACCTGCTGAAG |
| | CTGGCCGGCGACGTGGAGAGCAACCCCGGCCCCCCCGCCGAGCTGGAGG |
| | TGGAGTGCGCCACCCAGCTGAGAAGATTCGGCGACAAGCTGAACTTCAGA |
| | CAGAAGCTGCTGGGCAGCGGCGTGAAGCAGACCCTGAACTTCGACCTGCT |
| | GAAGCTGGCCGGCGACGTGGAGAGCAACCCCGGCCCCCCCGCCGAGCTG |
| | GAGGTGGAGTGCGCCACCCAGCTGAGAAGATTCGGCGACAAGCTGAACT |
| | TCAGACAGAAGCTGCTGGGCAGCGGCGTGAAGCAGACCCTGAACTTCGA |
| | CCTGCTGAAGCTGGCCGGCGACGTGGAGAGCAACCCCGGCCCCCCCGCCG |
| | AGCTGGAGGTGGAGTGCGCCACCCAGCTGAGAAGATTCGGCGACAAGCT |
| | GAACTTCAGACAGAAGCTGCTGGGCAGCGGCGTGAAGCAGACCCTGAAC |
| | TTCGACCTGCTGAAGCTGGCCGGCGACGTGGAGAGCAACCCCGGCCCCCC |
| | CGCCGAGCTGGAGGTGGAGTGCGCCACCCAGCTGAGAAGATTCGGCGAC |
| | AAGCTGAACTTCAGACAGAAGCTGCTGGGCAGCGGCGTGAAGCAGACCC |
| | TGAACTTCGACCTGCTGAAGCTGGCCGGCGACGTGGAGAGCAACCCCGGC |
| | CCCCCCGCCGAGCTGGAGGTGGAGTGCGCCACCCAGCTGAGAAGATTCGG |
| | CGACAAGCTGAACTTCAGACAGAAGCTGCTGGGCAGCGGCGTGAAGCAG |
| | ACCCTGAACTTCGACCTGCTGAAGCTGGCCGGCGACGTGGAGAGCAACCC |
| | CGGCCCCGGCAAGCCCATCCCCAACCCCCTGCTGGGCCTGGACAGCACC |
| | ((NoxaBH3.F2A)\hat{ }10_cV5, (NoxaBH3.F2A)\hat{ }10_cV5_DX) |
| 266 | ATGCCCGCCGAGCTGGAGGTGGAGTGCGCCACCCAGCTGAGAAGATTCG |
| | GCGACAAGCTGAACTTCAGACAGAAGCTGCTGGGCAGCGGCGTGAAGCA |
| | GACCCTGAACTTCGACCTGCTGAAGCTGGCCGGCGACGTGGAGAGCAACC |
| | CCGGCCCCAACCTGTGGGCCGCCCAGAGATACGGCAGAGAGCTGAGAAG |
| | AATGAGCGACGAGTTCGTGGACAGCTTCAAGAAGGGCGGCAGCGGCGTG |
| | AAGCAGACCCTGAACTTCGACCTGCTGAAGCTGGCCGGCGACGTGGAGA |
| | GCAACCCCGGCCCCCCCGCCGAGCTGGAGGTGGAGTGCGCCACCCAGCTG |
| | AGAAGATTCGGCGACAAGCTGAACTTCAGACAGAAGCTGCTGGGCAGCG |
| | GCGTGAAGCAGACCCTGAACTTCGACCTGCTGAAGCTGGCCGGCGACGTG |
| | GAGAGCAACCCCGGCCCCAACCTGTGGGCCGCCCAGAGATACGGCAGAG |
| | AGCTGAGAAGAATGAGCGACGAGTTCGTGGACAGCTTCAAGAAGGGCGG |
| | CAGCGGCGTGAAGCAGACCCTGAACTTCGACCTGCTGAAGCTGGCCGGCG |
| | ACGTGGAGAGCAACCCCGGCCCCCCCGCCGAGCTGGAGGTGGAGTGCGC |
| | CACCCAGCTGAGAAGATTCGGCGACAAGCTGAACTTCAGACAGAAGCTG |
| | CTGGGCAGCGGCGTGAAGCAGACCCTGAACTTCGACCTGCTGAAGCTGGC |
| | CGGCGACGTGGAGAGCAACCCCGGCCCCAACCTGTGGGCCGCCCAGAGA |
| | TACGGCAGAGAGCTGAGAAGAATGAGCGACGAGTTCGTGGACAGCTTCA |
| | AGAAGGGCGGCAGCGGCGTGAAGCAGACCCTGAACTTCGACCTGCTGAA |
| | GCTGGCCGGCGACGTGGAGAGCAACCCCGGCCCCCCCGCCGAGCTGGAG |
| | GTGGAGTGCGCCACCCAGCTGAGAAGATTCGGCGACAAGCTGAACTTCAG |
| | ACAGAAGCTGCTGGGCAGCGGCGTGAAGCAGACCCTGAACTTCGACCTGC |
| | TGAAGCTGGCCGGCGACGTGGAGAGCAACCCCGGCCCCAACCTGTGGGC |
| | CGCCCAGAGATACGGCAGAGAGCTGAGAAGAATGAGCGACGAGTTCGTG |
| | GACAGCTTCAAGAAGGGCGGCAGCGGCGTGAAGCAGACCCTGAACTTCG |
| | ACCTGCTGAAGCTGGCCGGCGACGTGGAGAGCAACCCCGGCCCCCCCGCC |
| | GAGCTGGAGGTGGAGTGCGCCACCCAGCTGAGAAGATTCGGCGACAAGC |
| | TGAACTTCAGACAGAAGCTGCTGGGCAGCGGCGTGAAGCAGACCCTGAA |
| | CTTCGACCTGCTGAAGCTGGCCGGCGACGTGGAGAGCAACCCCGGCCCCA |
| | ACCTGTGGGCCGCCCAGAGATACGGCAGAGAGCTGAGAAGAATGAGCGA |
| | CGAGTTCGTGGACAGCTTCAAGAAGGGCGGCAGCGGCGTGAAGCAGACC |
| | CTGAACTTCGACCTGCTGAAGCTGGCCGGCGACGTGGAGAGCAACCCCGG |
| | CCCCCCCGCCGAGCTGGAGGTGGAGTGCGCCACCCAGCTGAGAAGATTCG |
| | GCGACAAGCTGAACTTCAGACAGAAGCTGCTGGGCAGCGGCGTGAAGCA |
| | GACCCTGAACTTCGACCTGCTGAAGCTGGCCGGCGACGTGGAGAGCAACC |
| | CCGGCCCCAACCTGTGGGCCGCCCAGAGATACGGCAGAGAGCTGAGAAG |
| | AATGAGCGACGAGTTCGTGGACAGCTTCAAGAAGGGCGGCAGCGGCGTG |
| | AAGCAGACCCTGAACTTCGACCTGCTGAAGCTGGCCGGCGACGTGGAGA |
| | GCAACCCCGGCCCCCCCGCCGAGCTGGAGGTGGAGTGCGCCACCCAGCTG |
| | AGAAGATTCGGCGACAAGCTGAACTTCAGACAGAAGCTGCTGGGCAGCG |
| | GCGTGAAGCAGACCCTGAACTTCGACCTGCTGAAGCTGGCCGGCGACGTG |
| | GAGAGCAACCCCGGCCCCAACCTGTGGGCCGCCCAGAGATACGGCAGAG |
| | AGCTGAGAAGAATGAGCGACGAGTTCGTGGACAGCTTCAAGAAGGGCGG |
| | CAGCGGCGTGAAGCAGACCCTGAACTTCGACCTGCTGAAGCTGGCCGGCG |
| | ACGTGGAGAGCAACCCCGGCCCCCCCGCCGAGCTGGAGGTGGAGTGCGC |
| | CACCCAGCTGAGAAGATTCGGCGACAAGCTGAACTTCAGACAGAAGCTG |
| | CTGGGCAGCGGCGTGAAGCAGACCCTGAACTTCGACCTGCTGAAGCTGGC |
| | CGGCGACGTGGAGAGCAACCCCGGCCCCAACCTGTGGGCCGCCCAGAGA |
| | TACGGCAGAGAGCTGAGAAGAATGAGCGACGAGTTCGTGGACAGCTTCA |
| | AGAAGGGCGGCAGCGGCGTGAAGCAGACCCTGAACTTCGACCTGCTGAA |
| | GCTGGCCGGCGACGTGGAGAGCAACCCCGGCCCCCCCGCCGAGCTGGAG |
| | GTGGAGTGCGCCACCCAGCTGAGAAGATTCGGCGACAAGCTGAACTTCAG |
| | ACAGAAGCTGCTGGGCAGCGGCGTGAAGCAGACCCTGAACTTCGACCTGC |
| | TGAAGCTGGCCGGCGACGTGGAGAGCAACCCCGGCCCCAACCTGTGGGC |
| | CGCCCAGAGATACGGCAGAGAGCTGAGAAGAATGAGCGACGAGTTCGTG |
| | GACAGCTTCAAGAAGGGCGGCAGCGGCGTGAAGCAGACCCTGAACTTCG |
| | ACCTGCTGAAGCTGGCCGGCGACGTGGAGAGCAACCCCGGCCCCCCCGCC |
| | GAGCTGGAGGTGGAGTGCGCCACCCAGCTGAGAAGATTCGGCGACAAGC |
| | TGAACTTCAGACAGAAGCTGCTGGGCAGCGGCGTGAAGCAGACCCTGAA |
| | CTTCGACCTGCTGAAGCTGGCCGGCGACGTGGAGAGCAACCCCGGCCCCA |
| | ACCTGTGGGCCGCCCAGAGATACGGCAGAGAGCTGAGAAGAATGAGCGA |
| | CGAGTTCGTGGACAGCTTCAAGAAGGGCGGCAGCGGCGTGAAGCAGACC |
| | CTGAACTTCGACCTGCTGAAGCTGGCCGGCGACGTGGAGAGCAACCCCGG |
| | CCCCGGCAAGCCCATCCCCAACCCCCTGCTGGGCCTGGACAGCACC |
| | ((BadBH3.F2A)\hat{ }10_cV5,(BadBH3.F2A)\hat{ }10_cV5_DX) |
| 267 | ATGCCCGCCGAGCTGGAGGTGGAGTGCGCCACCCAGCTGAGAAGATTCG |
| | GCGACAAGCTGAACTTCAGACAGAAGCTGCTGGGCAGCGGCGTGAAGCA |
| | GACCCTGAACTTCGACCTGCTGAAGCTGGCCGGCGACGTGGAGAGCAACC |
| | CCGGCCCCAACCTGTGGGCCGCCCAGAGATACGGCAGAGAGCTGAGAAG |
| | AATGAGCGACGAGTTCGTGGACAGCTTCAAGAAGGGCGGCAGCGGCGTG |
| | AAGCAGACCCTGAACTTCGACCTGCTGAAGCTGGCCGGCGACGTGGAGA |
| | GCAACCCCGGCCCCCCCGCCGAGCTGGAGGTGGAGTGCGCCACCCAGCTG |
| | AGAAGATTCGGCGACAAGCTGAACTTCAGACAGAAGCTGCTGGGCAGCG |
| | GCGTGAAGCAGACCCTGAACTTCGACCTGCTGAAGCTGGCCGGCGACGTG |
| | GAGAGCAACCCCGGCCCCAACCTGTGGGCCGCCCAGAGATACGGCAGAG |
| | AGCTGAGAAGAATGAGCGACGAGTTCGTGGACAGCTTCAAGAAGGGCGG |
| | CAGCGGCGTGAAGCAGACCCTGAACTTCGACCTGCTGAAGCTGGCCGGCG |
| | ACGTGGAGAGCAACCCCGGCCCCCCCGCCGAGCTGGAGGTGGAGTGCGC |
| | CACCCAGCTGAGAAGATTCGGCGACAAGCTGAACTTCAGACAGAAGCTG |
| | CTGGGCAGCGGCGTGAAGCAGACCCTGAACTTCGACCTGCTGAAGCTGGC |
| | CGGCGACGTGGAGAGCAACCCCGGCCCCAACCTGTGGGCCGCCCAGAGA |
| | TACGGCAGAGAGCTGAGAAGAATGAGCGACGAGTTCGTGGACAGCTTCA |
| | AGAAGGGCGGCAGCGGCGTGAAGCAGACCCTGAACTTCGACCTGCTGAA |
| | GCTGGCCGGCGACGTGGAGAGCAACCCCGGCCCCCCCGCCGAGCTGGAG |
| | GTGGAGTGCGCCACCCAGCTGAGAAGATTCGGCGACAAGCTGAACTTCAG |
| | ACAGAAGCTGCTGGGCAGCGGCGTGAAGCAGACCCTGAACTTCGACCTGC |
| | TGAAGCTGGCCGGCGACGTGGAGAGCAACCCCGGCCCCAACCTGTGGGC |
| | CGCCCAGAGATACGGCAGAGAGCTGAGAAGAATGAGCGACGAGTTCGTG |
| | GACAGCTTCAAGAAGGGCGGCAGCGGCGTGAAGCAGACCCTGAACTTCG |
| | ACCTGCTGAAGCTGGCCGGCGACGTGGAGAGCAACCCCGGCCCCCCCGCC |
| | GAGCTGGAGGTGGAGTGCGCCACCCAGCTGAGAAGATTCGGCGACAAGC |
| | TGAACTTCAGACAGAAGCTGCTGGGCAGCGGCGTGAAGCAGACCCTGAA |
| | CTTCGACCTGCTGAAGCTGGCCGGCGACGTGGAGAGCAACCCCGGCCCCA |
| | ACCTGTGGGCCGCCCAGAGATACGGCAGAGAGCTGAGAAGAATGAGCGA |
| | CGAGTTCGTGGACAGCTTCAAGAAGGGCGGCAGCGGCGTGAAGCAGACC |
| | CTGAACTTCGACCTGCTGAAGCTGGCCGGCGACGTGGAGAGCAACCCCGG |
| | CCCCGGCAAGCCCATCCCCAACCCCCTGCTGGGCCTGGACAGCACC |
| | ((Noxa-F2A-BadBH3-F2A)\hat{ }5_cV5, (Noxa-F2A-BadBH3-F2A)\hat{ }5_cV5_DX) |
| 268 | ATGCCCGCCGAGCTGGAGGTGGAGTGCGCCACCCAGCTGAGAAGATTCG |
| | GCGACAAGCTGAACTTCAGACAGAAGCTGCTGGGCAGCGGCGTGAAGCA |
| | GACCCTGAACTTCGACCTGCTGAAGCTGGCCGGCGACGTGGAGAGCAACC |
| | CCGGCCCCAACCTGTGGGCCGCCCAGAGATACGGCAGAGAGCTGAGAAG |
| | AATGAGCGACGAGTTCGTGGACAGCTTCAAGAAGGGCGGCAGCGGCGTG |
| | AAGCAGACCCTGAACTTCGACCTGCTGAAGCTGGCCGGCGACGTGGAGA |
| | GCAACCCCGGCCCCCCCGCCGAGCTGGAGGTGGAGTGCGCCACCCAGCTG |
| | AGAAGATTCGGCGACAAGCTGAACTTCAGACAGAAGCTGCTGGGCAGCG |
| | GCGTGAAGCAGACCCTGAACTTCGACCTGCTGAAGCTGGCCGGCGACGTG |
| | GAGAGCAACCCCGGCCCCAACCTGTGGGCCGCCCAGAGATACGGCAGAG |
| | AGCTGAGAAGAATGAGCGACGAGTTCGTGGACAGCTTCAAGAAGGGCGG |
| | CAGCGGCGTGAAGCAGACCCTGAACTTCGACCTGCTGAAGCTGGCCGGCG |
| | ACGTGGAGAGCAACCCCGGCCCCCCCGCCGAGCTGGAGGTGGAGTGCGC |
| | CACCCAGCTGAGAAGATTCGGCGACAAGCTGAACTTCAGACAGAAGCTG |
| | CTGGGCAGCGGCGTGAAGCAGACCCTGAACTTCGACCTGCTGAAGCTGGC |
| | CGGCGACGTGGAGAGCAACCCCGGCCCCAACCTGTGGGCCGCCCAGAGA |
| | TACGGCAGAGAGCTGAGAAGAATGAGCGACGAGTTCGTGGACAGCTTCA |
| | AGAAGGGCGGCAGCGGCGTGAAGCAGACCCTGAACTTCGACCTGCTGAA |
| | GCTGGCCGGCGACGTGGAGAGCAACCCCGGCCCCCCCGCCGAGCTGGAG |
| | GTGGAGTGCGCCACCCAGCTGAGAAGATTCGGCGACAAGCTGAACTTCAG |
| | ACAGAAGCTGCTGGGCAGCGGCGTGAAGCAGACCCTGAACTTCGACCTGC |
| | TGAAGCTGGCCGGCGACGTGGAGAGCAACCCCGGCCCCAACCTGTGGGC |
| | CGCCCAGAGATACGGCAGAGAGCTGAGAAGAATGAGCGACGAGTTCGTG |
| | GACAGCTTCAAGAAGGGCGGCAGCGGCGTGAAGCAGACCCTGAACTTCG |
| | ACCTGCTGAAGCTGGCCGGCGACGTGGAGAGCAACCCCGGCCCCCCCGCC |
| | GAGCTGGAGGTGGAGTGCGCCACCCAGCTGAGAAGATTCGGCGACAAGC |
| | TGAACTTCAGACAGAAGCTGCTGGGCAGCGGCGTGAAGCAGACCCTGAA |
| | CTTCGACCTGCTGAAGCTGGCCGGCGACGTGGAGAGCAACCCCGGCCCCA |
| | ACCTGTGGGCCGCCCAGAGATACGGCAGAGAGCTGAGAAGAATGAGCGA |
| | CGAGTTCGTGGACAGCTTCAAGAAGGGCGGCAGCGGCGTGAAGCAGACC |
| | CTGAACTTCGACCTGCTGAAGCTGGCCGGCGACGTGGAGAGCAACCCCGG |
| | CCCCCCCGCCGAGCTGGAGGTGGAGTGCGCCACCCAGCTGAGAAGATTCG |
| | GCGACAAGCTGAACTTCAGACAGAAGCTGCTGGGCAGCGGCGTGAAGCA |
| | GACCCTGAACTTCGACCTGCTGAAGCTGGCCGGCGACGTGGAGAGCAACC |
| | CCGGCCCCAACCTGTGGGCCGCCCAGAGATACGGCAGAGAGCTGAGAAG |
| | AATGAGCGACGAGTTCGTGGACAGCTTCAAGAAGGGCGGCAGCGGCGTG |
| | AAGCAGACCCTGAACTTCGACCTGCTGAAGCTGGCCGGCGACGTGGAGA |
| | GCAACCCCGGCCCCCCCGCCGAGCTGGAGGTGGAGTGCGCCACCCAGCTG |
| | AGAAGATTCGGCGACAAGCTGAACTTCAGACAGAAGCTGCTGGGCAGCG |
| | GCGTGAAGCAGACCCTGAACTTCGACCTGCTGAAGCTGGCCGGCGACGTG |
| | GAGAGCAACCCCGGCCCCAACCTGTGGGCCGCCCAGAGATACGGCAGAG |
| | AGCTGAGAAGAATGAGCGACGAGTTCGTGGACAGCTTCAAGAAGGGCGG |
| | CAGCGGCGTGAAGCAGACCCTGAACTTCGACCTGCTGAAGCTGGCCGGCG |
| | ACGTGGAGAGCAACCCCGGCCCCCCCGCCGAGCTGGAGGTGGAGTGCGC |
| | CACCCAGCTGAGAAGATTCGGCGACAAGCTGAACTTCAGACAGAAGCTG |
| | CTGGGCAGCGGCGTGAAGCAGACCCTGAACTTCGACCTGCTGAAGCTGGC |
| | CGGCGACGTGGAGAGCAACCCCGGCCCCAACCTGTGGGCCGCCCAGAGA |
| | TACGGCAGAGAGCTGAGAAGAATGAGCGACGAGTTCGTGGACAGCTTCA |
| | AGAAGGGCGGCAGCGGCGTGAAGCAGACCCTGAACTTCGACCTGCTGAA |
| | GCTGGCCGGCGACGTGGAGAGCAACCCCGGCCCCCCCGCCGAGCTGGAG |
| | GTGGAGTGCGCCACCCAGCTGAGAAGATTCGGCGACAAGCTGAACTTCAG |
| | ACAGAAGCTGCTGGGCAGCGGCGTGAAGCAGACCCTGAACTTCGACCTGC |
| | TGAAGCTGGCCGGCGACGTGGAGAGCAACCCCGGCCCCAACCTGTGGGC |
| | CGCCCAGAGATACGGCAGAGAGCTGAGAAGAATGAGCGACGAGTTCGTG |
| | GACAGCTTCAAGAAGGGCGGCAGCGGCGTGAAGCAGACCCTGAACTTCG |
| | ACCTGCTGAAGCTGGCCGGCGACGTGGAGAGCAACCCCGGCCCCCCCGCC |
| | GAGCTGGAGGTGGAGTGCGCCACCCAGCTGAGAAGATTCGGCGACAAGC |
| | TGAACTTCAGACAGAAGCTGCTGGGCAGCGGCGTGAAGCAGACCCTGAA |
| | CTTCGACCTGCTGAAGCTGGCCGGCGACGTGGAGAGCAACCCCGGCCCCA |
| | ACCTGTGGGCCGCCCAGAGATACGGCAGAGAGCTGAGAAGAATGAGCGA |
| | CGAGTTCGTGGACAGCTTCAAGAAGGGCGGCAGCGGCGTGAAGCAGACC |
| | CTGAACTTCGACCTGCTGAAGCTGGCCGGCGACGTGGAGAGCAACCCCGG |
| | CCCCGGCAAGCCCATCCCCAACCCCCTGCTGGGCCTGGACAGCACC |
| | ((Noxa-F2A-BadBH3-F2A)\hat{ }10_cV5) |
| 269 | ATGGAGGAGCAGTGGGCTAGAGAAATTGGCGCGCAACTGAGACGCATGG |
| | CCGACGACCTTAACGCTCAATACGAAAGAAGGGGGTCGGGCGTGAAACA |
| | GACCTTGAATTTCGATCTGCTCAAACTGGCTGGTGATGTGGAGAGCAACC |
| | CGGGGCCTGACATGAGACCCGAAATATGGATTGCTCAGGAACTGCGGAG |
| | AATCGGAGACGAATTCAATGCGTATTATGCCAGACGCGGGAGCGGAGTG |
| | AAACAGACCCTTAACTTTGACCTCCTCAAGCTCGCAGGGGATGTGGAAAG |
| | CAATCCTGGTCCCGAAGAGCAATGGGCCAGAGAAATCGGCGCCCAACTG |
| | CGACGCATGGCCGATGATCTGAATGCCCAGTACGAACGTAGGGGTTCCGG |
| | TGTGAAGCAAACCCTCAACTTTGATTTATTAAAACTCGCCGGTGACGTCG |
| | AGAGCAATCCCGGCCCGGATATGCGCCCAGAAATTTGGATAGCCCAAGA |
| | GCTGAGAAGAATAGGCGACGAGTTTAACGCCTACTATGCCAGACGGGGC |
| | AGCGGCGTGAAACAAACTCTGAATTTCGACCTGCTGAAGCTAGCAGGCGA |
| | TGTGGAAAGCAATCCCGGGCCGGAAGAACAATGGGCCAGAGAAATCGGA |
| | GCCCAGCTGCGTAGAATGGCAGACGATTTAAACGCCCAGTATGAGAGGA |
| | GGGGCAGTGGCGTTAAACAGACCCTGAATTTTGATCTGCTGAAACTTGCC |
| | GGCGATGTGGAATCGAACCCCGGCCCCGATATGAGACCAGAGATCTGGAT |
| | CGCCCAGGAACTGAGGCGTATCGGTGATGAATTTAATGCCTACTATGCCA |
| | GGAGAGGCTCTGGTGTTAAGCAGACCCTGAATTTCGATCTGTTAAAGCTG |
| | GCCGGCGATGTGGAGTCCAACCCCGGGCCTGGAAAACCTATCCCCAACCC |
| | CCTACTGGGACTCGACAGCACC |
| | ((Puma-F2A-BimBH3-F2A)\hat{ }3_cV5_DX) |
| 270 | ATGCAATGGGCCAGAGAGATCGGAGCACAACTGCGGAGAATTGGAGAT |
| | GACCTGAACGCACAGTACGAACGCCGCAGACAGGGATCGGGCGTGAAG |
| | CAAACCCTCAATTTCGATCTTCTGAAATTGGCGGGCGACGTCGAGTCGA |
| | ACCCTGGTCCACAATGGGCGCGGGAAATCGGTGCCCAGCTGAGGAGAAT |
| | TGGCGATGACCTCAACGCCCAGTACGAAAGGCGGCGGCAGGGCAGCGG |
| | AGTGAAGCAGACTCTTAACTTCGACTTGCTGAAGCTGGCTGGGGACGTG |
| | GAATCCAACCCTGGACCCCAGTGGGCCCGCGAAATCGGCGCCCAGCTGC |
| | GGCGCATCGGGGACGACCTCAATGCGCAGTACGAGCGGCGGCGCCAGG |
| | GGTCCGGCGTCAAGCAGACCCTGAACTTCGACCTCCTCAAGCTGGCCGG |
| | AGATGTGGAGTCCAACCCGGGTCCCGGATCAGGGGTCAAGCAAACTCTG |
| | AACTTTGATCTGCTGAAGCTCGCTGGCGACGTGGAGAGCAACCCGGGAC |
| | CGGGAAAGCCTATTCCGAACCCCCTGCTGGGCCTGGACTCCACC |
| | ((SuperPumaBH3.F2A)\hat{ }3_cV5_DNA2.0) |
| 271 | ATGGAAGAACAATGGGCTAGGGAAATCGGAGCACAACTGAGAAGAATG |
| | GCAGACGATCTGAACGCCCAGTACGAGCGCCGGGGCTCAGGGGTCAAA |
| | CAGACCCTTAACTTCGATTTGCTGAAGCTCGCCGGGGATGTGGAGTCAA |
| | ATCCGGGGCCTGATATGCGGCCTGAAATTTGGATCGCACAGGAACTCCG |
| | CCGCATTGGGGATGAATTCAATGCGTACTATGCCAGGAGGGGAAGCGGC |
| | GTGAAGCAGACCCTGAACTTTGACTTGCTGAAACTGGCCGGTGACGTCG |
| | AGTCTAACCCTGGACCTGAGGAACAGTGGGCCAGGGAAATTGGAGCCCA |
| | GCTGCGGCGGATGGCCGATGACCTCAACGCCCAATACGAGCGCAGAGGC |
| | AGCGGGGTCAAGCAGACTCTCAATTTTGATCTCCTCAAGCTGGCCGGCG |
| | ATGTCGAGTCCAATCCGGGACCGGACATGAGGCCGGAGATTTGGATTGC |
| | CCAAGAGCTGCGCCGGATCGGGGACGAATTCAACGCCTACTACGCGCGG |
| | CGGGGTTCCGGAGTGAAGCAAACTTTGAATTTCGACCTTCTGAAATTGG |
| | CCGGCGACGTGGAGTCGAACCCGGGTCCGGAGGAGCAGTGGGCCCGCG |
| | AGATCGGCGCGCAGCTCAGACGCATGGCCGACGACCTGAACGCACAGTA |
| | CGAACGCAGGGGCTCGGGAGTTAAGCAGACCTTGAACTTCGATCTCCTG |
| | AAGCTTGCTGGAGATGTGGAATCCAACCCTGGTCCGGACATGCGGCCAG |
| | AAATCTGGATCGCCCAGGAACTGCGGCGCATCGGCGACGAGTTCAACGC |
| | TTACTACGCCCGCCGGGGTTCGGGCGTGAAACAAACCCTGAATTTCGAT |
| | CTTCTGAAGCTGGCGGGGGACGTGGAGAGCAACCCCGGACCCGGAAAG |
| | CCCATCCCAAACCCCCTGCTGGGTCTGGACTCCACC |
| | ((Puma-F2A-BimBH3-F2A)\hat{ }3_cV5_DX_DNA2.0) |
| 272 | ATGCAATGGGCCAGAGAAATTGGAGCGCAACTCAGAAGAATCGGCGAC |
| | GATCTGAACGCTCAGTACGAAAGACGCCGCCAGGGATCCGGCGTGAAGC |
| | AAACTCTGAACTTTGACCTCCTTAAGCTCGCCGGGGATGTCGAGAGCAA |
| | TCCTGGACCGCAGTGGGCCAGGGAGATCGGTGCCCAGTTGCGGCGGATC |
| | GGCGATGATCTCAACGCCCAATATGAACGGCGGAGACAGGGAAGCGGA |
| | GTCAAGCAAACCTTGAACTTCGACCTCCTGAAGCTGGCTGGCGACGTCG |
| | AATCCAACCCTGGACCCCAGTGGGCGCGGGAAATTGGTGCCCAGCTGCG |
| | ACGGATCGGGGATGACCTCAACGCACAATACGAGAGGCGGAGGCAGGG |
| | TTCCGGCGTCAAGCAGACTCTTAATTTCGATCTCCTGAAATTGGCCGGCG |
| | ATGTGGAGTCAAACCCGGGCCCGCAATGGGCCCGCGAGATCGGCGCCCA |
| | ACTGCGGCGCATTGGCGACGACCTGAACGCCCAGTACGAGCGCAGACGC |
| | CAGGGGTCCGGAGTGAAGCAGACCCTCAACTTCGATTTGTTGAAGCTCG |
| | CGGGCGACGTGGAAAGCAATCCGGGACCACAGTGGGCTAGAGAGATCG |
| | GAGCCCAGCTGAGGCGCATTGGTGACGACCTCAATGCACAGTACGAACG |
| | GAGGCGGCAGGGTTCGGGGGTGAAACAGACCCTGAACTTCGATCTGCTC |
| | AAGCTGGCGGGAGATGTGGAATCGAACCCAGGGCCGGGAAAGCCTATT |
| | CCCAACCCTCTGCTGGGCCTGGATTCCACC |
| | ((SuperPumaBH3.F2A)\hat{ }5_cV5_DNA2.0) |
| 273 | ATGGAAGAACAATGGGCACGGGAAATTGGAGCGCAGCTGAGGAGAATG |
| | GCTGATGACCTGAACGCTCAATACGAACGCCGCGGATCCGGCGTGAAGC |
| | AGACTCTTAACTTCGACTTGCTCAAGCTCGCTGGCGATGTCGAGTCCAAC |
| | CCGGGCCCCGAGGAGCAATGGGCCCGCGAAATTGGGGCCCAGCTCCGGC |
| | GCATGGCGGACGATCTCAACGCGCAGTATGAACGGAGGGGCTCCGGAGT |
| | CAAGCAGACCCTCAACTTCGACCTCCTGAAGCTCGCCGGCGACGTGGAG |
| | AGCAACCCTGGTCCCGAAGAACAGTGGGCCAGAGAAATCGGTGCCCAG |
| | CTTCGCCGGATGGCCGACGACTTGAACGCCCAATACGAGCGGAGAGGCT |
| | CGGGAGTGAAGCAAACCCTGAACTTCGATCTGCTTAAGCTGGCTGGGGA |
| | CGTCGAATCCAACCCCGGACCTGAGGAGCAGTGGGCCCGGGAAATCGG |
| | GGCCCAACTGAGAAGGATGGCAGACGACCTGAATGCCCAGTACGAGCG |
| | CCGCGGCTCAGGAGTGAAACAGACCTTGAACTTTGACTTGCTGAAGCTG |
| | GCCGGGGACGTGGAAAGCAACCCGGGGCCAGAGGAACAGTGGGCGCGG |
| | GAGATCGGTGCTCAGCTGAGAAGAATGGCAGATGATCTGAATGCGCAGT |
| | ACGAACGGCGCGGATCTGGAGTCAAACAGACCCTGAATTTCGACCTGTT |
| | GAAACTGGCCGGAGATGTGGAATCAAACCCCGGCCCTGGAAAGCCGATC |
| | CCGAACCCACTGCTGGGGCTCGACTCCACC |
| | ((pumaBH3.F2A)\hat{ }5_cV5_DNA2.0) |
| 274 | ATGCAATGGGCACGAGAGATTGGAGCACAGGAACGGAGAGAAGCCGAT |
| | GACGAGAACGCGCAGTACGAACGGCGGCGCCAGGGTTCCGGCGTGAAA |
| | CAGACTCTCAACTTCGACTTGCTGAAGCTCGCCGGGGACGTGGAATCCA |
| | ATCCAGGACCTCAATGGGCCCGGGAAATCGGCGCGCAGGAAAGACGCG |
| | AAGCCGACGATGAAAACGCCCAATACGAGCGGCGCAGACAGGGGTCGG |
| | GCGTGAAGCAAACGCTGAATTTCGACCTCCTGAAGTTGGCCGGAGATGT |
| | CGAGTCCAACCCTGGACCCCAGTGGGCCAGAGAGATTGGTGCCCAAGAA |
| | AGGAGAGAAGCGGATGACGAAAACGCTCAGTACGAGAGGCGCAGACAA |
| | GGCTCGGGAGTCAAGCAGACTCTGAACTTCGATCTTCTGAAGCTGGCTG |
| | GCGATGTGGAAAGCAACCCAGGACCGCAGTGGGCGCGCGAGATCGGAG |
| | CCCAGGAGAGGAGGGAGGCCGACGACGAAAATGCACAGTACGAAAGAA |
| | GGCGCCAGGGCTCCGGAGTGAAGCAGACCCTCAACTTTGACTTGCTCAA |
| | GCTGGCCGGCGACGTGGAGAGCAACCCTGGCCCGCAATGGGCTAGAGA |
| | AATCGGAGCTCAGGAGCGGCGGGAAGCAGACGACGAGAATGCGCAGTA |
| | TGAGCGCCGGCGGCAGGGAAGCGGTGTCAAGCAAACCCTGAACTTTGAT |
| | CTGCTCAAACTGGCCGGGGATGTGGAATCGAACCCCGGACCGGGGAAGC |
| | CCATCCCCAACCCGCTTCTGGGCCTGGACTCCACC |
| | ((KittyCatBH3.F2A)\hat{ }5_cV5_DNA2.0) |
| 275 | ATGCAGTGGGCTAGGGAAATCGGTGCACAAGAGAGAAGGGAAGCGGACG |
| | ACGAGAACGCACAGTACGAGCGCCGCCGGCAGGGGAGCGGAGTCAAACA |
| | GACCCTTAACTTTGATCTGCTGAAACTCGCCGGCGACGTGGAATCCAACC |
| | CGGGTCCGCAGTGGGCGCGCGAAATTGGGGCGCAGGAGCGCCGCGAAGC |
| | GGATGACGAAAACGCTCAGTACGAACGTCGGCGGCAGGGAAGCGGCGTG |
| | AAGCAGACGCTGAATTTTGACCTTTTGAAACTGGCCGGAGATGTGGAGAG |
| | CAACCCTGGACCGCAATGGGCCAGAGAGATTGGGGCCCAAGAACGGCGA |
| | GAAGCCGACGACGAGAACGCCCAGTACGAACGCCGGCGCCAGGGCTCTG |
| | GAGTGAAACAGACCTTGAACTTCGACCTCTTGAAATTGGCCGGAGATGTC |
| | GAATCAAACCCTGGTCCTCAGTGGGCCCGGGAGATTGGCGCACAAGAGC |
| | GCAGAGAAGCTGACGATGAAAATGCGCAATACGAAAGACGGCGCCAAGG |
| | TTCTGGCGTGAAACAAACTCTCAACTTCGACTTGCTGAAGCTTGCGGGGG |
| | ATGTGGAATCAAACCCGGGACCCCAGTGGGCGAGGGAAATAGGGGCACA |
| | GGAAAGAAGAGAAGCAGATGATGAAAACGCCCAATATGAGCGCCGGCGG |
| | CAAGGGTCCGGAGTGAAGCAAACTTTGAATTTCGATCTCCTCAAGCTGGC |
| | GGGGGACGTCGAGTCTAATCCTGGGCCACAATGGGCCCGCGAAATTGGA |
| | GCACAGGAACGCCGCGAGGCCGACGACGAGAACGCCCAATATGAAAGAA |
| | GGCGCCAGGGATCCGGAGTCAAGCAAACACTGAACTTTGACCTCTTGAAG |
| | CTCGCTGGCGATGTCGAGTCCAATCCCGGCCCACAGTGGGCGCGCGAGAT |
| | TGGAGCTCAAGAGCGGCGGGAAGCAGACGACGAAAACGCGCAGTATGAA |
| | CGCCGGAGACAGGGCTCGGGGGTCAAGCAGACCCTGAACTTCGATCTGCT |
| | AAAGCTGGCCGGTGACGTCGAGTCTAACCCCGGTCCTCAATGGGCACGGG |
| | AAATCGGGGCGCAAGAGCGGCGCGAGGCAGACGACGAGAACGCGCAATA |
| | TGAACGCAGACGGCAGGGGTCCGGCGTCAAGCAGACTTTAAATTTCGACC |
| | TCCTCAAGTTGGCAGGGGATGTGGAATCGAACCCCGGACCACAGTGGGCC |
| | AGGGAGATCGGAGCCCAGGAACGGAGAGAAGCCGATGACGAGAATGCCC |
| | AGTACGAAAGGCGCCGCCAGGGGTCGGGGGTGAAACAGACTTTGAACTT |
| | CGACCTTCTGAAACTGGCTGGGGACGTTGAGAGCAATCCGGGACCTCAGT |
| | GGGCCCGAGAGATCGGCGCCCAGGAAAGAAGAGAGGCTGATGACGAAAA |
| | TGCCCAATACGAGCGTCGCAGACAGGGTTCGGGTGTGAAGCAGACTCTGA |
| | ACTTCGATCTGCTCAAGCTCGCCGGGGACGTGGAGTCGAACCCGGGGCCC |
| | CAATGGGCGAGAGAAATTGGTGCCCAGGAGCGAAGAGAGGCCGATGATG |
| | AGAACGCTCAATACGAACGCAGGCGACAGGGCTCCGGTGTCAAGCAAAC |
| | CCTCAACTTCGATTTGCTCAAGCTTGCCGGGGATGTCGAAAGCAACCCGG |
| | GCCCGGGGAAGCCGATCCCGAACCCCCTGCTGGGTCTGGATTCCACT |
| | (KittyCatBH3.F2A)\hat{ }10_cV5_DNA2.0) |
| 276 | ATGGAAGAACAATGGGCAAGGGAAATCGGGGCCCAGTTGCGAAGAATGG |
| | CTGACGACCTCAATGCTCAATACGAACGCAGGGGATCTGGAGTCAAACAA |
| | ACCCTGAACTTCGACCTCCTGAAACTCGCGGGGGATGTGGAATCGAACCC |
| | AGGTCCAGAGGAGCAATGGGCCCGCGAAATCGGTGCGCAGCTGCGTCGC |
| | ATGGCAGATGACCTTAACGCACAATACGAACGGCGCGGATCCGGGGTTA |
| | AGCAAACTCTGAATTTCGATCTCTTGAAATTGGCGGGAGATGTGGAAAGC |
| | AACCCTGGACCCGAGGAGCAGTGGGCTCGGGAAATCGGAGCGCAGTTAC |
| | GGCGCATGGCGGACGACCTGAACGCGCAATACGAAAGACGCGGGTCGGG |
| | AGTCAAGCAGACCTTGAACTTTGACTTGCTGAAGCTCGCGGGCGATGTGG |
| | AGTCCAACCCGGGTCCGGAGGAGCAATGGGCACGGGAGATTGGAGCCCA |
| | ACTGCGACGCATGGCCGACGATCTGAACGCCCAATACGAGCGGCGGGGC |
| | TCCGGTGTCAAGCAAACCCTCAACTTCGATTTGCTCAAACTGGCCGGGGA |
| | TGTCGAGTCGAACCCCGGACCGGAAGAACAGTGGGCGAGAGAGATTGGT |
| | GCACAGCTCAGAAGAATGGCCGATGATCTCAACGCGCAGTACGAACGGA |
| | GAGGTTCCGGCGTGAAGCAGACCCTCAATTTTGACCTCCTGAAGCTTGCC |
| | GGCGATGTCGAGAGCAACCCCGGCCCCGAGGAACAGTGGGCCCGGGAAA |
| | TTGGGGCACAGCTGCGCCGGATGGCGGACGATCTCAATGCGCAGTACGAG |
| | CGGAGAGGATCAGGGGTGAAGCAAACTCTCAACTTTGATCTGCTGAAGCT |
| | GGCCGGTGACGTGGAGTCTAACCCAGGGCCCGAGGAACAGTGGGCCCGG |
| | GAGATCGGCGCCCAGCTGAGGCGGATGGCCGATGACTTGAACGCCCAGT |
| | ACGAAAGGCGGGGATCCGGAGTAAAGCAGACTTTGAATTTCGACCTTCTT |
| | AAGTTGGCCGGGGACGTGGAATCAAACCCAGGACCGGAGGAACAGTGGG |
| | CCAGGGAGATCGGTGCCCAGCTCAGGAGGATGGCTGACGACCTCAACGC |
| | GCAGTATGAAAGAAGAGGGTCCGGAGTGAAGCAGACTCTTAACTTCGATC |
| | TCCTGAAGCTGGCTGGTGATGTGGAGAGCAATCCGGGGCCCGAAGAACA |
| | ATGGGCCAGAGAAATTGGCGCGCAGTTGCGGCGGATGGCTGATGACCTG |
| | AACGCACAGTACGAGAGGCGGGGCAGCGGAGTGAAACAGACCCTGAATT |
| | TTGATCTTCTGAAGTTGGCGGGCGACGTGGAATCCAACCCCGGGCCGGAA |
| | GAACAGTGGGCACGCGAGATTGGCGCTCAGCTGCGCAGAATGGCAGACG |
| | ACTTGAACGCTCAGTACGAACGCCGGGGCTCGGGGGTGAAGCAGACGCT |
| | GAACTTTGATCTCCTCAAACTCGCCGGCGACGTCGAAAGCAATCCTGGCC |
| | CCGGAAAGCCCATCCCCAACCCCCTGCTGGGTCTGGACTCCACC |
| | ((pumaBH3.F2A)\hat{ }10_cV5_DNA2.0) |
| 277 | ATGGAAGAACAATGGGCGCGGGAAATCGGGGCGCAACTGAGAAGAATGG |
| | CAGATGATCTCAATGCCCAGTATGAACGCCGGGGTAGCGGGGTCAAGCA |
| | GACTTTGAATTTCGATCTCCTTAAGCTCGCTGGAGATGTGGAGTCCAACCC |
| | TGGACCCGATATGAGGCCTGAAATCTGGATCGCGCAGGAACTGAGGCGC |
| | ATCGGCGATGAATTCAATGCATACTACGCCCGCCGGGGCTCTGGCGTTAA |
| | GCAAACACTGAACTTCGACCTCCTGAAGCTGGCCGGGGATGTGGAAAGTA |
| | ATCCGGGTCCCGAAGAACAATGGGCACGGGAAATTGGAGCCCAGCTCCG |
| | CCGGATGGCAGACGATCTGAACGCACAATACGAGAGGCGGGGGTCGGGA |
| | GTGAAGCAAACCCTGAACTTTGACTTGCTTAAGCTGGCTGGGGACGTGGA |
| | GAGCAATCCTGGACCAGACATGCGCCCAGAGATTTGGATTGCTCAGGAAC |
| | TGCGGAGAATCGGGGATGAGTTCAATGCGTATTACGCCCGGCGGGGCTCC |
| | GGCGTGAAGCAGACTCTGAACTTCGACCTTCTGAAGTTGGCTGGCGATGT |
| | GGAATCAAACCCCGGCCCGGAGGAACAGTGGGCGAGGGAAATCGGCGCT |
| | CAGCTTAGACGGATGGCCGACGACCTCAACGCGCAATACGAGCGCCGGG |
| | GCTCAGGCGTGAAGCAGACGCTCAACTTTGACCTCTTGAAACTGGCGGGG |
| | GACGTGGAGTCGAACCCGGGACCTGATATGCGGCCGGAGATCTGGATTGC |
| | GCAGGAGCTGCGCCGCATCGGAGATGAATTCAACGCCTATTACGCGCGCA |
| | GAGGATCCGGCGTCAAACAGACTCTCAATTTCGACCTCCTGAAGCTGGCC |
| | GGTGACGTGGAATCCAATCCCGGCCCTGAAGAACAGTGGGCCAGAGAGA |
| | TCGGTGCTCAGCTTCGCCGCATGGCCGATGACCTGAACGCTCAGTACGAA |
| | CGGCGGGGATCCGGAGTAAAACAGACCCTGAATTTTGATCTGCTGAAGCT |
| | CGCTGGCGACGTCGAATCGAACCCTGGCCCGGATATGCGCCCTGAAATCT |
| | GGATCGCTCAGGAGCTCCGCCGCATTGGCGACGAATTTAACGCCTACTAT |
| | GCCCGGCGCGGATCGGGGGTGAAACAGACTCTTAATTTCGACTTGCTCAA |
| | ACTTGCGGGAGATGTCGAATCTAACCCCGGACCAGAGGAGCAATGGGCC |
| | CGCGAGATCGGAGCACAGCTGCGGAGGATGGCTGACGACTTGAACGCGC |
| | AGTATGAGCGCCGCGGTTCGGGGGTCAAACAAACTCTAAACTTCGACTTA |
| | CTGAAGCTTGCCGGCGACGTGGAAAGCAACCCTGGTCCCGACATGCGGCC |
| | CGAAATTTGGATCGCCCAGGAGCTGAGGAGAATCGGCGACGAGTTCAAC |
| | GCGTACTACGCCAGACGCGGCTCCGGAGTCAAGCAAACTCTTAACTTCGA |
| | TCTGTTGAAACTCGCGGGTGATGTCGAGAGCAACCCGGGCCCTGGAAAGC |
| | CGATCCCTAACCCGCTGCTCGGCCTGGACTCCACT |
| | ((Puma-F2A-BimBH3-F2A)\hat{ }5_cV5_DNA2.0) |
| 278 | ATGCAATGGGCACGAGAGATTGGTGCTCAACTCAGACGGATTGGTGACGA |
| | CCTCAACGCCCAGTACGAGCGGAGGCGGCAAGGCTCCGGTGTGAAGCAA |
| | ACCCTGAACTTCGATTTGCTCAAGCTGGCTGGAGATGTCGAGAGCAACCC |
| | CGGGCCTCAATGGGCGAGGGAGATCGGAGCCCAGCTTAGAAGGATAGGC |
| | GACGATCTGAATGCACAGTATGAAAGACGGCGGCAGGGATCCGGAGTCA |
| | AACAGACTCTGAACTTTGACCTCCTGAAGCTGGCCGGAGATGTGGAAAGC |
| | AACCCGGGTCCTCAGTGGGCACGCGAAATCGGAGCTCAGCTGCGCAGGAT |
| | TGGTGATGATCTGAACGCACAGTACGAACGACGCCGCCAGGGGAGCGGA |
| | GTCAAGCAAACCTTGAACTTTGATCTGCTGAAGCTTGCCGGCGATGTGGA |
| | ATCCAACCCTGGACCCCAGTGGGCTCGCGAAATTGGCGCCCAGCTGAGGA |
| | GGATCGGCGACGACTTGAACGCCCAATATGAACGCCGGAGGCAGGGCTC |
| | CGGAGTGAAACAAACCCTCAACTTTGATTTGCTTAAGTTGGCCGGGGACG |
| | TGGAGTCTAACCCGGGCCCCCAGTGGGCCAGAGAGATCGGCGCGCAACTC |
| | AGAAGGATTGGCGACGACCTGAACGCGCAGTATGAGAGGCGGAGACAGG |
| | GCAGCGGCGTGAAGCAGACCTTAAACTTCGACCTGTTGAAGTTGGCTGGC |
| | GATGTCGAATCGAACCCTGGCCCACAATGGGCCCGGGAGATTGGAGCTCA |
| | GTTGCGGAGAATCGGTGATGACCTGAATGCGCAATATGAACGCAGACGG |
| | CAGGGGTCCGGGGTGAAACAGACCCTCAATTTCGACTTGCTGAAGCTCGC |
| | CGGGGATGTGGAATCGAATCCGGGTCCACAATGGGCCCGCGAAATCGGG |
| | GCCCAACTCCGCCGGATCGGCGATGATCTCAACGCACAATACGAACGCCG |
| | CCGGCAGGGCTCGGGCGTGAAGCAAACTCTGAATTTCGATCTTCTTAAGC |
| | TCGCTGGTGATGTGGAGTCAAACCCCGGCCCGCAATGGGCGCGCGAAATC |
| | GGAGCACAACTTCGGCGGATCGGAGATGACCTTAATGCTCAATACGAGAG |
| | GCGGCGGCAAGGTTCAGGGGTGAAGCAAACGCTGAACTTTGACCTCCTCA |
| | AGTTGGCGGGCGACGTCGAAAGCAATCCTGGTCCGCAGTGGGCCAGAGA |
| | GATCGGCGCTCAGCTCCGACGCATTGGCGATGACCTTAACGCACAGTACG |
| | AACGGCGCAGACAGGGCTCAGGAGTGAAGCAGACTCTCAACTTCGATCTG |
| | CTCAAACTGGCGGGTGACGTGGAGAGTAACCCGGGACCTCAATGGGCGA |
| | GAGAAATCGGTGCCCAGCTGCGCCGCATCGGGGACGATCTCAATGCCCAA |
| | TACGAAAGACGGCGCCAAGGATCCGGGGTCAAGCAGACGCTTAATTTCG |
| | ACCTCTTGAAGCTGGCTGGTGACGTCGAGTCCAACCCAGGCCCTGGAAAG |
| | CCCATCCCCAACCCGCTGCTCGGACTGGACTCAACT |
| | ((SuperPumaBH3.F2A)\hat{ }10_cV5_DNA2.0) |
| 279 | ATGCAGTGGGCCAGAGAGATTGGTGCCCAAGAAAGGAGAGAAGCGGAT |
| | GACGAAAACGCTCAGTACGAGAGGCGCAGACAAGGCTCGGGAGTCAAG |
| | CAGACTCTGAACTTCGATCTTCTGAAGCTGGCTGGCGATGTGGAAAGCA |
| | ACCCAGGACCGCAGTGGGCGCGCGAGATCGGAGCCCAGGAGAGGAGGG |
| | AGGCCGACGACGAAAATGCACAGTACGAAAGAAGGCGCCAGGGCTCCG |
| | GAGTGAAGCAGACCCTCAACTTTGACTTGCTCAAGCTGGCCGGCGACGT |
| | GGAGAGCAACCCTGGCCCGCAATGGGCTAGAGAAATCGGAGCTCAGGA |
| | GCGGCGGGAAGCAGACGACGAGAATGCGCAGTATGAGCGCCGGCGGCA |
| | GGGAAGCGGTGTCAAGCAAACCCTGAACTTTGATCTGCTCAAACTGGCC |
| | GGGGATGTGGAATCGAACCCCGGACCGGGGAAGCCCATCCCCAACCCGC |
| | TTCTGGGCCTGGACTCCACC |
| | ((KittyCatBH3.F2A)\hat{ }3_cV5) |
| 280 | ATGCAGTGGGCCAGAGAGATTGGTGCCCAAGAAAGGAGAGAAGCGGAT |
| | GACGAAAACGCTCAGTACGAGAGGCGCAGACAAGGAAGCGGAGCTACT |
| | AACTTCAGCCTGCTGAAGCAGGCTGGAGACGTGGAGGAGAACCCTGGAC |
| | CTCAGTGGGCGCGCGAGATCGGAGCCCAGGAGAGGAGGGAGGCCGACG |
| | ACGAAAATGCACAGTACGAAAGAAGGCGCCAGGGAAGCGGAGCTACTA |
| | ACTTCAGCCTGCTGAAGCAGGCTGGAGACGTGGAGGAGAACCCTGGACC |
| | TCAATGGGCTAGAGAAATCGGAGCTCAGGAGCGGCGGGAAGCAGACGA |
| | CGAGAATGCGCAGTATGAGCGCCGGCGGCAGGGAAGCGGTGTCAAGCA |
| | AACCCTGAACTTTGATCTGCTCAAACTGGCCGGGGATGTGGAATCGAAC |
| | CCCGGACCGGGGAAGCCCATCCCCAACCCGCTTCTGGGCCTGGACTCCA |
| | CC |
| | ((KittyCatBH3.P2A)\hat{ }3_cV5) |
| 281 | ATGGAGGAGCAATGGGCTAGAGAGATCGGCGCACAGCTGCGGCGCATG |
| | GCCGATGATCTGAACGCCCAATACGAGAGGAGAGGAAGCGGAGCTACT |
| | AACTTCAGCCTGCTGAAGCAGGCTGGAGACGTGGAGGAGAACCCTGGAC |
| | CTGAGGAACAATGGGCGCGCGAAATCGGTGCCCAGCTCCGCCGGATGGC |
| | AGACGACCTGAACGCGCAGTACGAGCGGCGGGGAAGCGGAGCTACTAA |
| | CTTCAGCCTGCTGAAGCAGGCTGGAGACGTGGAGGAGAACCCTGGACCT |
| | GAAGAACAGTGGGCCAGGGAAATTGGAGCTCAGCTGCGGAGAATGGCC |
| | GACGACCTCAACGCCCAGTACGAACGGCGCGGAAAACCTATCCCGAACC |
| | CACTCTTGGGCCTGGACTCCACC |
| | (PumaBH3(x3.P2A).v5_miR122) |
| 282 | ATGGAGGAGCAATGGGCTAGAGAGATCGGCGCACAGCTGCGGCGCATG |
| | GCCGATGATCTGAACGCCCAATACGAGAGGAGAGGTGGCGGAAGTGGT |
| | GGCGGAAGTGGTGGCGGAAGTGAGGAGCAATGGGCTAGAGAGATCGGC |
| | GCACAGCTGCGGCGCATGGCCGATGATCTGAACGCCCAATACGAGAGGA |
| | GAGGTGGCGGAAGTGGTGGCGGAAGTGGTGGCGGAAGTGAGGAGCAAT |
| | GGGCTAGAGAGATCGGCGCACAGCTGCGGCGCATGGCCGATGATCTGAA |
| | CGCCCAATACGAGCGGCGCGGAAAACCTATCCCGAACCCACTCTTGGGC |
| | CTGGACTCCACC |
| | ((PUMA BH3.GGGSx3)x3.V5) |
| 283 | ATGGAGGAGCAGTGGGCTAGAGAGATCGGGGCACAGCTGCGGCGCATG |
| | GCTGACGACCTGAACGCTCAATACGAGCGCAGAGGAGGCGGATCCGGT |
| | GGCGGAAGCGGTGGAGGAAGTGAGGAGCAATGGGCTAGAGAGATCGGA |
| | GCACAGCTGCGGCGCATGGCCGACGATCTTAACGCACAATACGAAAGGA |
| | GAGGGGGCGGAAGTGGTGGCGGAAGTGGCGGCGGAAGTGAGGAGCAAT |
| | GGGCTAGAGAAATCGGCGCACAGCTGCGGCGCATGGCCGATGATCTGAA |
| | TGCCCAGTACGAACGGCGCGGAAAACCTATCCCGAACCCACTCTTGGGA |
| | CTGGACTCCACC |
| | ((PUMA BH3.GGGSx3)x3.V5_DX) |
| 284 | ATGGAGGAGCAATGGGCTAGAGAGATCGGCGCACAGCTGCGGCGCATG |
| | GCCGATGATCTGAACGCCCAATACGAGAGGAGAGGTGGCGGAAGTGGT |
| | GGCGGAAGTGGTGGCGGAAGTGAGGAGCAATGGGCTAGAGAGATCGGC |
| | GCACAGCTGCGGCGCATGGCCGATGATCTGAACGCCCAATACGAGAGGA |
| | GAGGTGGCGGAAGTGGTGGCGGAAGTGGTGGCGGAAGTGAGGAGCAAT |
| | GGGCTAGAGAGATCGGCGCACAGCTGCGGCGCATGGCCGATGATCTGAA |
| | CGCCCAATACGAGCGGCGCGGAAAACCTATCCCGAACCCACTCTTGGGC |
| | CTGGACTCCACC |
| | (PUMA BH3.GGGSx3.PUMA BH3.V5) |
| 285 | ATGGAGGAGCAATGGGCTAGAGAGATCGGCGCACAGCTGCGGCGCATG |
| | GCCGATGATCTGAACGCCCAATACGAGAGGAGAGGAAGCGGAGCTACT |
| | AACTTCAGCCTGCTGAAGCAGGCTGGAGACGTGGAGGAGAACCCTGGAC |
| | CTGAGGAACAATGGGCGCGCGAAATCGGTGCCCAGCTCCGCCGGATGGC |
| | AGACGACCTGAACGCGCAGTACGAGCGGCGGGGAAGCGGAGCTACTAA |
| | CTTCAGCCTGCTGAAGCAGGCTGGAGACGTGGAGGAGAACCCTGGACCT |
| | GAAGAACAGTGGGCCAGGGAAATTGGAGCTCAGCTGCGGAGAATGGCC |
| | GACGACCTCAACGCCCAGTACGAACGGCGCGGAAAACCTATCCCGAACC |
| | CACTCTTGGGCCTGGACTCCACC |
| | (PumaBH3(x3.P2A).v5_miR122) |
| 286 | MGNRSSHSRLGRIEADSESQEDIIRNIARHLAQVGDSMDRSIPPGMDYKDDDD |
| | K |
| | (hum.BID(61-104).FLAG) |
| 287 | ATGGGCAACCGCAGCAGCCACTCCCGCTTGGGAAGAATAGAGGCAGATT |
| | CTGAAAGTCAAGAAGACATCATCCGGAATATTGCCAGGCACCTCGCCCAG |
| | GTCGGGGACAGCATGGACCGTAGCATCCCTCCGGGCATGGATTACAAGGA |
| | CGATGATGACAAG |
| | (hum.BID(61-104).FLAG) |
| 288 | MGNRSSHSRLGRIEADSESQEDIIRNIARHLAQVGDSMDRSIPPGM |
| | (hum.BID(61-104) without FLAG tag) |
| 289 | MGNRSSHSRLGRIEADSESQEDIIRNIARHLAQVGDSMDRSIPPGLVNGLALQ |
| | LRNTSRSEEDRNRDLATALEQLLQAYPRDMEKEKTMLVLALLLAKKVASHT |
| | PSLLRDVFHTTVNFINQNLRTYVRSLARNGMDMDYKDDDDK |
| | (hum.tBID(61-195).FLAG) |
| 290 | ATGGGCAACCGCAGCAGCCACTCCCGCTTGGGAAGAATAGAGGCAGATT |
| | CTGAAAGTCAAGAAGACATCATCCGGAATATTGCCAGGCACCTCGCCCAG |
| | GTCGGGGACAGCATGGACCGTAGCATCCCTCCGGGCCTGGTGAACGGCCT |
| | GGCCCTGCAGCTCAGGAACACCAGCCGGTCGGAGGAGGACCGGAACAGG |
| | GACCTGGCCACTGCCCTGGAGCAGCTGCTGCAGGCCTACCCTAGAGACAT |
| | GGAGAAGGAGAAGACCATGCTGGTGCTGGCCCTGCTGCTGGCCAAGAAG |
| | GTGGCCAGTCACACGCCGTCCTTGCTCCGTGATGTCTTTCACACAACAGTG |
| | AATTTTATTAACCAGAACCTACGCACCTACGTGAGGAGCTTAGCCAGAAA |
| | TGGGATGGACATGGATTACAAGGACGATGATGACAAG |
| | (hum.tBID(61-195).FLAG) |
| 291 | MGNRSSHSRLGRIEADSESQEDIIRNIARHLAQVGDSMDRSIPPGLVNGLALQ |
| | LRNTSRSEEDRNRDLATALEQLLQAYPRDMEKEKTMLVLALLLAKKVASHT |
| | PSLLRDVFHTTVNFINQNLRTYVRSLARNGMDM |
| | (hum.tBID(61-195) without FLAG tag) |
| 292 | MESQEDIRNIARHLAQVGDSMDRSIPPGLVNGLALQLRNTSRSEEDRNRDLA |
| | TALEQLLQAYPRDMEKEKTMLVLALLLAKKVASHTPSLLRDVFHTTVNFINQ |
| | NLRTYVRSLARNGMDMDYKDDDDK |
| | (hum.tBID(77-195).FLAG) |
| 293 | ATGGAAAGTCAAGAAGACATCATCCGGAATATTGCCAGGCACCTCGCCCA |
| | GGTCGGGGACAGCATGGACCGTAGCATCCCTCCGGGCCTGGTGAACGGCC |
| | TGGCCCTGCAGCTCAGGAACACCAGCCGGTCGGAGGAGGACCGGAACAG |
| | GGACCTGGCCACTGCCCTGGAGCAGCTGCTGCAGGCCTACCCTAGAGACA |
| | TGGAGAAGGAGAAGACCATGCTGGTGCTGGCCCTGCTGCTGGCCAAGAA |
| | GGTGGCCAGTCACACGCCGTCCTTGCTCCGTGATGTCTTTCACACAACAGT |
| | GAATTTTATTAACCAGAACCTACGCACCTACGTGAGGAGCTTAGCCAGAA |
| | ATGGGATGGACATGGATTACAAGGACGATGATGACAAG |
| | (hum.tBID(77-195).FLAG) |
| 294 | MESQEDIIRNIARHLAQVGDSMDRSIPPGLVNGLALQLRNTSRSEEDRNRDLA |
| | TALEQLLQAYPRDMEKEKTMLVLALLLAKKVASHTPSLLRDVFHTTVNFINQ |
| | NLRTYVRSLARNGMDM |
| | (hum.tBID(77-195) without FLAG tag) |
| 295 | UGUAGUGUUUCCUACUUUAUGGA |
| | (miR-142-3p) |
| 296 | CAUAAAGUAGAAAGCACUACU |
| | (miR-142-5p) |
| 297 | GACAGUGCAGUCACCCAUAAAGUAGAAAGCACUACUAACAGCACUGGA |
| | GGGUGUAGUGUUUCCUACUUUAUGGAUGAGUGUACUGUG |
| | (miR-142) |
| 298 | UCCAUAAAGUAGGAAACACUACA |
| | (miR-142-3p binding site) |
| 299 | AGUAGUGCUUUCUACUUUAUG |
| | (miR-142-5p binding site) |
| 300 | ATGGAGGAGCAGTGGGCGCGGGAGATAGGGGCCCAGCTAAGGCGGATGG |
| | CCGACGACCTAAACGCCCAATACGAAAGGAGGGGCTCGGGGGTCAAACA |
| | GACCCTCAATTTCGACCTCCTCAAGCTCGCGGGAGACGTCGAGAGCAACC |
| | CCGGCCCCGAGGAGCAGTGGGCGCGCGAAATAGGGGCCCAGCTCCGGCG |
| | CATGGCCGACGACCTCAACGCGCAATACGAGAGGCGCGGCAGCGGGGTA |
| | AAGCAAACGTTGAACTTCGACCTCCTCAAGCTCGCAGGGGACGTGGAGTC |
| | CAACCCCGGGCCCGAAGAACAATGGGCCCGGGAAATCGGCGCCCAGCTG |
| | CGCCGTATGGCTGACGACCTCAACGCGCAGTATGAACGCCGGGGGAAGC |
| | CCATCCCCAACCCCCTGCTCGGCCTCGATAGCACG |
| | (Puma-BH3 Multimers Codon optimized) |
| 301 | ATGGAGGAGCAGTGGGCCCGCGAGATAGGCGCCCAGCTCCGTAGGATGG |
| | CGGACGATCTAAACGCCCAGTACGAGAGGCGGGGCAGCGGGGTCAAACA |
| | GACATTGAATTTCGACCTCTTGAAGCTCGCCGGCGACGTGGAGAGCAACC |
| | CCGGGCCCGAGGAGCAGTGGGCGCGGGAGATCGGAGCCCAACTCAGGAG |
| | AATGGCCGACGACCTCAACGCCCAGTACGAGCGACGCGGTAGCGGGGTA |
| | AAGCAAACCCTCAACTTCGACCTCCTCAAGCTCGCCGGGGACGTTGAGTC |
| | CAATCCCGGGCCCGAAGAACAGTGGGCCCGGGAGATAGGCGCCCAGCTG |
| | CGTCGTATGGCCGACGATCTGAACGCCCAGTACGAGCGGAGAGGGAAGC |
| | CCATCCCGAACCCGTTGCTGGGGCTGGACAGCACC |
| | (Puma-BH3 Multimers Codon optimized) |
| 302 | ATGGAGGAACAGTGGGCCCGCGAGATCGGCGCCCAACTCCGGCGGATGG |
| | CCGACGATCTCAACGCCCAGTACGAGAGGCGGGGCTCCGGGGTTAAGCA |
| | AACCCTCAATTTCGACCTCCTCAAGCTTGCCGGGGACGTCGAAAGTAACC |
| | CCGGCCCGGAGGAACAGTGGGCCCGGGAGATAGGGGCGCAGCTACGCAG |
| | GATGGCCGACGATCTCAACGCCCAGTACGAGAGGAGGGGGTCGGGGGTC |
| | AAGCAGACCCTCAACTTCGACCTACTCAAGCTCGCCGGCGACGTGGAGAG |
| | CAACCCCGGGCCCGAAGAACAGTGGGCCCGGGAGATCGGGGCCCAGCTG |
| | AGACGGATGGCCGATGACCTGAACGCTCAGTACGAGCGGCGTGGGAAGC |
| | CCATCCCCAACCCCCTGCTGGGTTTAGACAGCACC |
| | (Puma-BH3 Multimers Codon optimized) |
| 303 | ATGGAGGAACAGTGGGCCCGCGAGATCGGCGCCCAGCTCCGCCGCATGG |
| | CGGACGATCTTAACGCCCAATACGAGAGGAGGGGGTCCGGCGTCAAGCA |
| | GACCCTCAACTTCGACCTCCTCAAACTCGCCGGAGACGTCGAGTCCAACC |
| | CCGGTCCCGAAGAACAGTGGGCCCGGGAAATCGGGGCCCAGCTCCGCCG |
| | CATGGCAGACGATCTCAACGCCCAGTACGAGCGGCGCGGGTCCGGGGTC |
| | AAGCAGACTCTCAACTTCGATCTTCTCAAGTTAGCGGGGGACGTGGAGTC |
| | CAATCCAGGTCCGGAGGAGCAGTGGGCCCGGGAGATAGGGGCCCAGCTC |
| | CGCCGAATGGCCGACGACCTGAACGCTCAATATGAGCGCCGGGGGAAAC |
| | CCATCCCCAACCCGCTGCTCGGGCTGGATAGCACT |
| | (Puma-BH3 Multimers Codon optimized) |
| 304 | ATGGAGGAGCAGTGGGCAAGGGAGATAGGAGCTCAGCTCAGGCGGATGG |
| | CCGACGACCTCAACGCGCAGTACGAACGGCGGGGATCCGGAGTCAAACA |
| | GACATTGAATTTCGACCTTCTCAAACTCGCCGGCGACGTTGAGAGCAATC |
| | CCGGGCCCGAGGAACAGTGGGCGCGGGAAATCGGCGCCCAGCTAAGGCG |
| | GATGGCCGACGACCTAAACGCCCAATACGAGCGGCGGGGGTCCGGCGTG |
| | AAGCAGACCCTAAACTTCGACCTCCTGAAGCTTGCCGGGGACGTGGAGAG |
| | CAATCCCGGCCCCGAAGAACAGTGGGCCCGGGAGATCGGGGCCCAGCTG |
| | CGGCGCATGGCTGACGACCTCAACGCCCAGTACGAGCGGCGGGGGAAGC |
| | CCATCCCCAACCCGCTCCTGGGTCTGGACAGCACA |
| | (Puma-BH3 Multimers Codon optimized) |
| 305 | ATGGAGGAACAGTGGGCCAGGGAAATCGGGGCCCAGCTAAGGAGGATGG |
| | CCGACGACCTAAACGCCCAGTACGAACGGCGAGGTAGCGGGGTCAAGCA |
| | GACTCTCAACTTCGACCTCTTGAAACTCGCCGGGGACGTCGAGTCGAATC |
| | CAGGCCCCGAGGAGCAGTGGGCACGAGAAATAGGGGCCCAGCTACGCCG |
| | CATGGCGGACGACCTCAACGCTCAATACGAGCGAAGAGGATCCGGCGTA |
| | AAACAGACGTTGAACTTCGACCTCCTCAAGCTCGCCGGGGACGTAGAGTC |
| | CAATCCGGGCCCTGAGGAACAGTGGGCCCGGGAGATCGGGGCCCAGCTG |
| | CGCCGAATGGCGGACGATCTGAATGCCCAGTATGAGAGGAGGGGGAAGC |
| | CCATCCCAAATCCACTGCTGGGTCTGGATTCGACA |
| | (Puma-BH3 Multimers Codon optimized) |
| 306 | ATGGAGGAGCAGTGGGCGCGAGAGATCGGCGCCCAGCTCCGTAGGATGG |
| | CAGACGACTTAAACGCCCAATACGAACGCCGGGGGAGCGGCGTCAAACA |
| | GACGCTCAACTTCGACTTACTAAAACTAGCCGGCGACGTTGAGAGCAATC |
| | CCGGGCCCGAGGAGCAGTGGGCCCGGGAGATAGGCGCGCAGCTTCGCCG |
| | CATGGCGGACGACCTCAACGCCCAATACGAGCGCCGCGGGTCCGGGGTC |
| | AAGCAGACGCTCAACTTCGACCTCCTCAAACTGGCCGGAGACGTGGAGAG |
| | CAACCCCGGCCCCGAGGAGCAGTGGGCCCGCGAAATCGGGGCCCAGCTG |
| | CGCAGAATGGCGGACGACCTGAACGCGCAGTATGAGCGACGGGGGAAGC |
| | CCATCCCGAACCCCCTGCTCGGACTCGACTCCACT |
| | (Puma-BH3 Multimers Codon optimized) |
| 307 | ATGGAGGAGCAGTGGGCCAGGGAGATCGGCGCACAGCTCCGCCGCATGG |
| | CGGACGACCTCAACGCCCAATACGAACGACGGGGGTCCGGGGTCAAACA |
| | GACGCTCAACTTCGACCTCCTTAAACTCGCCGGCGACGTAGAGTCTAACC |
| | CCGGCCCCGAGGAGCAGTGGGCCCGGGAGATAGGGGCCCAGCTCCGGCG |
| | GATGGCCGACGATCTCAACGCCCAGTACGAGCGTAGGGGGAGCGGCGTT |
| | AAGCAAACGCTTAATTTCGACCTCCTCAAGCTCGCGGGCGACGTCGAGTC |
| | AAACCCCGGGCCAGAGGAGCAGTGGGCCCGTGAGATCGGTGCCCAGCTG |
| | AGGCGAATGGCCGATGACCTGAACGCCCAGTATGAGCGCCGTGGGAAGC |
| | CCATTCCGAATCCTCTCCTGGGTCTGGACAGCACC |
| | (Puma-BH3 Multimers Codon optimized) |
| 308 | ATGGAGGAACAGTGGGCTCGCGAGATCGGGGCTCAGCTCCGTAGGATGG |
| | CCGACGATCTCAACGCCCAGTACGAGCGCAGGGGGAGCGGCGTCAAGCA |
| | GACCTTGAATTTCGACCTCCTCAAGCTCGCCGGAGACGTCGAGTCCAACC |
| | CAGGGCCCGAGGAGCAGTGGGCCCGCGAGATCGGAGCCCAGCTCCGGAG |
| | GATGGCAGACGACTTGAACGCACAGTACGAGCGCCGGGGGTCCGGGGTT |
| | AAGCAAACCCTCAACTTCGACCTCCTTAAGCTGGCAGGCGACGTGGAGTC |
| | GAATCCCGGGCCCGAGGAGCAGTGGGCCAGGGAGATCGGCGCACAGCTG |
| | CGGCGCATGGCCGACGACCTGAACGCGCAGTATGAGCGCCGAGGTAAGC |
| | CCATCCCCAACCCCCTGCTTGGGCTGGACTCCACC |
| | (Puma-BH3 Multimers Codon optimized) |
| 309 | ATGGAGGAGCAGTGGGCCCGAGAGATCGGCGCCCAGCTCAGGCGGATGG |
| | CCGACGACCTTAACGCCCAGTACGAGCGGCGGGGGAGCGGGGTCAAGCA |
| | GACCCTTAATTTCGACCTTCTCAAACTGGCCGGGGACGTCGAGTCGAACC |
| | CCGGGCCAGAGGAGCAGTGGGCCAGGGAGATCGGAGCCCAATTACGACG |
| | GATGGCCGACGACCTCAACGCCCAATACGAGCGGAGGGGGTCCGGAGTC |
| | AAACAGACCCTCAACTTCGATCTCTTGAAGCTCGCAGGAGACGTCGAAAG |
| | CAATCCCGGGCCCGAAGAACAGTGGGCCCGGGAGATAGGGGCACAGCTC |
| | CGCAGGATGGCCGACGATCTGAACGCCCAGTACGAGCGTAGGGGTAAAC |
| | CTATCCCAAACCCACTTCTGGGGCTGGACAGCACT |
| | (Puma-BH3 Multimers Codon optimized) |
| 310 | ATGGAAGAACAGTGGGCTCGCGAGATCGGCGCTCAGCTCCGACGGATGG |
| | CCGACGACTTGAACGCGCAGTACGAGCGCCGGGGGAGCGGAGTCAAGCA |
| | GACACTCAACTTCGACCTCCTAAAGTTGGCGGGCGACGTGGAGAGCAACC |
| | CGGGGCCCGAGGAGCAGTGGGCGAGGGAGATAGGCGCCCAGCTGCGCCG |
| | GATGGCCGACGACTTGAACGCTCAATACGAGCGGAGGGGGTCCGGCGTC |
| | AAGCAGACGCTTAATTTCGACCTCCTCAAGCTCGCCGGCGACGTGGAATC |
| | CAACCCCGGCCCGGAGGAGCAGTGGGCCCGAGAAATCGGAGCCCAACTG |
| | CGGAGGATGGCTGACGACCTGAACGCCCAGTACGAGCGCCGAGGAAAGC |
| | CGATCCCCAACCCCCTGCTGGGACTGGACAGCACG |
| | (Puma-BH3 Multimers Codon optimized) |
| 311 | ATGGAGGAGCAGTGGGCCCGGGAAATCGGGGCCCAGTTACGCAGGATGG |
| | CCGACGATCTAAACGCCCAATACGAGAGGAGGGGCTCGGGGGTAAAACA |
| | GACCCTCAATTTCGATTTGCTCAAACTCGCCGGCGACGTCGAGAGTAACC |
| | CGGGCCCCGAGGAGCAGTGGGCCCGCGAGATCGGGGCGCAGCTCCGGCG |
| | GATGGCAGACGACCTCAACGCGCAGTACGAACGCCGGGGCTCCGGCGTC |
| | AAGCAAACGTTGAACTTCGACCTCCTCAAACTCGCCGGGGACGTAGAGAG |
| | CAACCCCGGGCCCGAGGAGCAGTGGGCTCGTGAGATTGGCGCCCAGCTAC |
| | GCCGTATGGCCGACGACCTCAACGCCCAGTACGAGAGGAGGGGTAAGCC |
| | GATCCCCAACCCCCTGCTGGGGCTGGACTCCACC |
| | (Puma-BH3 Multimers Codon optimized) |
| 312 | ATGGAGGAACAGTGGGCGCGAGAGATCGGGGCCCAGCTCAGGCGGATGG |
| | CCGACGATCTCAACGCCCAGTACGAACGGAGGGGTAGCGGGGTAAAGCA |
| | AACTCTAAACTTCGATCTCCTCAAGCTCGCCGGCGACGTAGAGTCCAATC |
| | CGGGGCCCGAGGAGCAGTGGGCGCGGGAGATCGGCGCCCAGCTCCGGAG |
| | GATGGCAGACGATCTCAACGCCCAGTACGAGCGGAGAGGCAGCGGGGTC |
| | AAACAGACCCTCAACTTCGATCTCCTAAAGCTCGCCGGGGACGTGGAGAG |
| | CAACCCCGGGCCCGAGGAGCAGTGGGCCCGCGAAATCGGTGCCCAGCTTC |
| | GACGTATGGCCGATGATCTGAACGCCCAATACGAGCGGCGCGGCAAACC |
| | CATTCCCAATCCGCTGCTCGGGCTGGACTCCACC |
| | (Puma-BH3 Multimers Codon optimized) |
| 313 | ATGGAGGAGCAGTGGGCCCGGGAAATCGGAGCCCAACTACGGCGCATGG |
| | CCGACGACCTCAACGCCCAATACGAGCGGAGGGGCTCGGGAGTCAAGCA |
| | GACTCTAAATTTCGACCTCCTCAAGCTCGCGGGCGACGTCGAGTCCAACC |
| | CCGGTCCCGAAGAACAGTGGGCACGAGAGATCGGCGCCCAGCTCCGGCG |
| | AATGGCGGACGACCTTAACGCCCAGTACGAGCGGCGGGGGAGCGGGGTC |
| | AAGCAAACACTCAACTTCGACCTACTCAAGCTCGCCGGGGACGTCGAGAG |
| | CAATCCCGGGCCCGAGGAACAGTGGGCCAGGGAGATTGGGGCCCAGCTG |
| | AGGAGGATGGCGGACGACCTGAACGCCCAGTACGAGAGGCGAGGCAAGC |
| | CGATCCCCAATCCCCTGCTGGGCCTGGATTCCACC |
| | (Puma-BH3 Multimers Codon optimized) |
| 314 | ATGGAGGAGCAGTGGGCGCGCGAGATAGGCGCCCAACTCCGTAGGATGG |
| | CCGACGATCTTAACGCCCAGTACGAGCGCCGGGGTAGCGGGGTGAAGCA |
| | GACCCTCAACTTCGACCTTCTCAAGCTTGCCGGGGACGTAGAAAGCAATC |
| | CCGGGCCCGAGGAGCAGTGGGCCAGGGAAATCGGGGCCCAGCTCCGCCG |
| | TATGGCCGACGACCTCAACGCGCAGTACGAGCGCCGAGGGTCGGGAGTC |
| | AAGCAGACCCTCAACTTCGATCTCCTCAAGCTCGCCGGCGACGTGGAAAG |
| | CAACCCGGGCCCCGAAGAACAGTGGGCCCGGGAGATTGGGGCACAGCTG |
| | AGGAGGATGGCCGACGATCTGAACGCCCAGTACGAACGGCGGGGCAAGC |
| | CCATCCCAAACCCGCTGCTAGGACTGGACTCAACG |
| | (Puma-BH3 Multimers Codon optimized) |
| 315 | ATGGAGGAGCAGTGGGCACGAGAAATCGGCGCCCAGCTTCGTCGGATGG |
| | CCGACGATCTCAACGCGCAGTACGAGAGGCGGGGCTCGGGAGTCAAACA |
| | GACCCTCAACTTCGACCTCCTCAAGCTCGCCGGCGACGTCGAGTCCAACC |
| | CGGGCCCGGAAGAACAGTGGGCCAGAGAGATCGGGGCCCAGCTAAGGCG |
| | TATGGCCGACGATCTCAACGCCCAGTACGAGCGGAGGGGCTCCGGCGTCA |
| | AGCAGACCCTTAATTTCGATCTCTTGAAGCTCGCCGGGGACGTCGAAAGC |
| | AATCCCGGGCCCGAGGAACAGTGGGCCCGGGAAATCGGTGCACAGCTCA |
| | GGCGCATGGCGGATGATCTCAACGCCCAATACGAGCGCCGGGGCAAACC |
| | CATACCTAACCCCCTGCTCGGTCTGGACTCCACC |
| | (Puma-BH3 Multimers Codon optimized) |
| 316 | ATGGAGGAGCAGTGGGCGCGGGAGATCGGGGCGCAGCTAAGGAGGATGG |
| | CGGACGATCTCAACGCGCAGTACGAAAGGCGCGGCAGCGGCGTGAAGCA |
| | GACGCTCAACTTCGACCTACTCAAGCTCGCGGGGGACGTCGAATCGAACC |
| | CCGGCCCGGAGGAACAGTGGGCCAGGGAGATCGGCGCCCAGCTACGGCG |
| | TATGGCCGACGACCTCAACGCCCAATACGAGAGGAGGGGGTCGGGAGTC |
| | AAACAGACCCTAAACTTCGACCTCCTCAAGCTCGCCGGGGACGTCGAGTC |
| | CAACCCCGGTCCCGAGGAGCAGTGGGCCAGGGAAATCGGGGCGCAACTG |
| | CGCCGCATGGCCGACGATCTGAACGCCCAGTATGAGCGCAGGGGCAAGC |
| | CGATCCCGAATCCGCTGCTAGGTCTGGACTCCACC |
| | (Puma-BH3 Multimers Codon optimized) |
| 317 | ATGGAGGAGCAGTGGGCCCGCGAGATCGGCGCACAGCTCCGACGAATGG |
| | CCGACGATCTCAACGCCCAATACGAACGGCGGGGGAGCGGAGTCAAGCA |
| | GACTTTAAACTTCGACCTCCTCAAGCTTGCCGGGGACGTGGAAAGTAACC |
| | CCGGACCGGAGGAGCAGTGGGCCCGCGAGATAGGAGCGCAGCTCAGGCG |
| | CATGGCCGACGATCTCAACGCCCAATACGAGCGGAGGGGAAGCGGGGTA |
| | AAACAGACGCTCAACTTCGACCTCCTCAAATTAGCCGGCGACGTGGAGAG |
| | CAACCCCGGGCCCGAGGAGCAGTGGGCCCGCGAGATAGGGGCCCAACTG |
| | CGGCGCATGGCGGACGACCTGAACGCCCAGTACGAGAGGCGGGGCAAGC |
| | CGATCCCTAACCCCCTGCTGGGGTTGGACTCCACC |
| | (Puma-BH3 Multimers Codon optimized) |
| 318 | ATGGAAGAACAGTGGGCACGGGAGATCGGCGCACAGCTAAGGAGGATGG |
| | CCGACGACCTTAACGCGCAGTACGAGCGCAGAGGGAGCGGCGTCAAGCA |
| | GACGCTCAATTTCGACCTTCTCAAGCTCGCGGGGGACGTTGAGTCCAATC |
| | CCGGACCCGAGGAGCAGTGGGCCCGCGAGATAGGGGCCCAGCTCCGGCG |
| | GATGGCAGACGATCTCAACGCCCAATACGAGAGGAGGGGGTCGGGGGTC |
| | AAACAGACCCTTAACTTCGACCTCCTCAAGCTCGCCGGGGACGTCGAGAG |
| | TAACCCCGGACCGGAGGAGCAGTGGGCCCGGGAAATTGGCGCCCAACTC |
| | AGGCGGATGGCCGATGATCTCAACGCCCAGTACGAACGTCGGGGTAAGC |
| | CCATCCCGAACCCCCTGCTGGGGCTGGACTCGACC |
| | (Puma-BH3 Multimers Codon optimized) |
| 319 | ATGGAGGAGCAGTGGGCAAGGGAGATAGGCGCACAGCTCCGTCGGATGG |
| | CCGACGACCTGAACGCCCAATACGAGCGGAGAGGGTCCGGGGTCAAGCA |
| | GACCCTCAATTTCGACTTGCTCAAGCTGGCAGGGGACGTCGAAAGCAACC |
| | CCGGCCCGGAGGAGCAGTGGGCGCGCGAGATCGGCGCCCAGCTTAGGCG |
| | GATGGCCGACGACTTAAACGCGCAATACGAGCGCCGCGGCAGCGGGGTC |
| | AAACAGACCCTAAACTTCGACCTCCTCAAGCTCGCCGGCGACGTGGAGAG |
| | CAACCCCGGCCCCGAAGAACAGTGGGCCCGCGAGATCGGGGCGCAGCTG |
| | CGTAGAATGGCCGACGATCTGAACGCCCAGTATGAGAGGCGGGGCAAAC |
| | CTATCCCGAATCCACTGCTGGGCCTGGACAGCACA |
| | (Puma-BH3 Multimers Codon optimized) |
| 320 | ATGGAGGAACAGTGGGCTCGCGAGATAGGCGCCCAGCTCCGCAGAATGG |
| | CCGACGATCTTAACGCCCAATACGAACGGCGGGGGTCCGGGGTCAAGCA |
| | GACGTTAAACTTCGACCTCCTCAAACTCGCCGGGGACGTGGAGTCCAACC |
| | CCGGGCCCGAGGAGCAGTGGGCGCGCGAGATCGGGGCCCAGCTCCGACG |
| | GATGGCCGACGACCTCAACGCGCAGTACGAGCGCAGAGGAAGCGGGGTC |
| | AAGCAGACCCTCAACTTCGATCTCCTCAAGTTGGCGGGCGACGTTGAAAG |
| | CAACCCCGGACCGGAGGAGCAATGGGCCCGCGAGATCGGGGCCCAACTC |
| | AGGAGGATGGCGGACGACCTGAACGCCCAGTACGAACGGAGGGGGAAAC |
| | CTATCCCCAACCCTCTACTGGGGCTGGACTCTACG |
| | (Puma-BH3 Multimers Codon optimized) |
| 321 | ATGGAGGAACAGTGGGCCCGCGAGATCGGCGCCCAACTCCGTAGGATGG |
| | CCGACGATCTCAACGCCCAGTACGAGAGGAGGGGGAGCGGGGTCAAGCA |
| | GACGCTCAACTTCGACCTCCTCAAGCTCGCCGGGGACGTCGAGTCCAACC |
| | CGGGTCCAGAGGAGCAGTGGGCGAGGGAAATCGGCGCCCAGCTCCGTCG |
| | GATGGCCGACGACCTAAACGCGCAGTACGAGAGGAGGGGTTCCGGCGTT |
| | AAACAAACGCTCAACTTCGACCTCCTCAAACTCGCCGGGGACGTCGAGAG |
| | CAACCCCGGACCCGAGGAGCAGTGGGCTCGGGAGATTGGGGCCCAGCTG |
| | AGGCGGATGGCCGATGACCTGAATGCGCAGTACGAGCGCCGCGGAAAAC |
| | CCATCCCTAACCCGCTGCTCGGCCTGGACTCCACC |
| | (Puma-BH3 Multimers Codon optimized) |
| 322 | ATGGAGGAGCAGTGGGCCCGAGAAATAGGGGCCCAGCTCAGGAGGATGG |
| | CCGACGACCTCAACGCCCAATACGAAAGGAGGGGGTCGGGCGTCAAGCA |
| | GACCCTTAATTTCGACTTGCTTAAGCTTGCCGGGGACGTAGAATCCAACC |
| | CGGGACCCGAGGAGCAGTGGGCCCGAGAAATCGGAGCCCAGCTCCGCCG |
| | AATGGCGGACGATCTCAACGCCCAATACGAGAGGAGGGGATCCGGCGTC |
| | AAGCAGACGCTCAATTTCGACCTCCTCAAACTCGCCGGCGACGTTGAATC |
| | AAACCCGGGGCCGGAAGAACAGTGGGCCAGAGAGATCGGCGCACAGCTG |
| | CGCCGAATGGCCGATGACCTGAACGCCCAGTACGAGCGCCGGGGCAAGC |
| | CCATACCGAACCCCCTCCTGGGCCTGGACTCCACC |
| | (Puma-BH3 Multimers Codon optimized) |
| 323 | ATGGAGGAGCAGTGGGCCCGCGAAATCGGCGCCCAGCTCCGGAGAATGG |
| | CCGACGACCTTAACGCCCAGTACGAAAGGAGGGGCAGCGGGGTCAAACA |
| | GACGCTAAACTTCGACCTCCTCAAGCTCGCCGGGGACGTTGAGTCCAACC |
| | CCGGGCCGGAGGAACAGTGGGCGCGGGAGATCGGGGCGCAGCTTAGGCG |
| | AATGGCCGACGACCTAAACGCCCAGTACGAGCGCAGGGGGTCGGGCGTC |
| | AAGCAGACCCTCAACTTCGACCTCCTTAAACTCGCGGGGGACGTCGAGAG |
| | CAATCCGGGGCCGGAAGAACAGTGGGCTCGGGAGATTGGCGCCCAGCTG |
| | CGGCGCATGGCCGATGACCTGAACGCCCAGTATGAACGCCGCGGTAAGCC |
| | CATCCCGAACCCGCTGCTGGGTCTGGATAGCACC |
| | (Puma-BH3 Multimers Codon optimized) |
| 324 | ATGGAGGAACAGTGGGCCCGGGAGATCGGCGCCCAGCTCAGGCGGATGG |
| | CGGACGACCTCAACGCCCAGTACGAGCGGAGGGGGAGCGGGGTCAAGCA |
| | AACCCTCAATTTCGACCTCCTCAAGTTGGCCGGCGACGTGGAGTCGAACC |
| | CCGGGCCCGAGGAACAGTGGGCCCGCGAGATAGGGGCACAGCTCCGCAG |
| | GATGGCCGACGACCTTAACGCGCAGTACGAGAGGAGGGGCTCGGGAGTT |
| | AAGCAGACCCTCAATTTCGATCTCCTCAAACTAGCCGGGGACGTAGAAAG |
| | CAACCCCGGCCCCGAGGAGCAGTGGGCCCGAGAAATCGGCGCGCAGCTG |
| | AGAAGGATGGCTGACGACCTGAACGCGCAGTATGAGAGACGGGGGAAGC |
| | CGATCCCCAACCCCCTCCTCGGGTTGGACTCCACC |
| | (Puma-BH3 Multimers Codon optimized) |
| 325 | GGGAAATAAGAGAGAAAAGAAGAGTAAGAAGAAATATAAGAGCCACC |
| | (5′ UTR) |
| 326 | TGATAATAGTCCATAAAGTAGGAAACACTACAGCTGGAGCCTCGGTGGCC |
| | ATGCTTCTTGCCCCTTGGGCCCAAACACCATTGTCACACTCCATCCCCCCA |
| | GCCCCTCCTCCCCTTCCTGCACCCGTACCCCCGTGGTCTTTGAATAAAGTC |
| | TGAGTGGGCGGC |
| | (3′ UTR with miR-142-3p (underlined) and miR-122-5p (double underlined) |
| | binding sites) |
| 327 | GGGAAATAAGAGAGAAAAGAAGAGTAAGAAGAAATATAAGAGCCACC |
| | (5′UTR-001) |
| 328 | GGGAGATCAGAGAGAAAAGAAGAGTAAGAAGAAATATAAGAGCCACC |
| | (5′UTR-002) |
| 329 | GGAATAAAAGTCTCAACACAACATATACAAAACAAACGAATCTCAAGCA |
| | ATCAAGCATTCTACTTCTATTGCAGCAATTTAAATCATTTCTTTTAAAGCA |
| | AAAGCAATTTTCTGAAAATTTTCACCATTTACGAACGATAGCAAC |
| | (5′UTR-003) |
| 330 | GGGAGACAAGCUUGGCAUUCCGGUACUGUUGGUAAAGCCACC |
| | (5′UTR-004) |
| 331 | GGGAGATCAGAGAGAAAAGAAGAGTAAGAAGAAATATAAGAGCCACC |
| | (5′UTR-005) |
| 332 | GGAATAAAAGTCTCAACACAACATATACAAAACAAACGAATCTCAAGCA |
| | ATCAAGCATTCTACTTCTATTGCAGCAATTTAAATCATTTCTTTTAAAGCA |
| | AAAGCAATTTTCTGAAAATTTTCACCATTTACGAACGATAGCAAC |
| | (5′UTR-006) |
| 333 | GGGAGACAAGCUUGGCAUUCCGGUACUGUUGGUAAAGCCACC |
| | (5′UTR-007) |
| 334 | GGGAATTAACAGAGAAAAGAAGAGTAAGAAGAAATATAAGAGCCACC |
| | (5′UTR-008) |
| 335 | GGGAAATTAGACAGAAAAGAAGAGTAAGAAGAAATATAAGAGCCACC |
| | (5′UTR-009) |
| 336 | GGGAAATAAGAGAGTAAAGAACAGTAAGAAGAAATATAAGAGCCACC |
| | (5′UTR-010) |
| 337 | GGGAAAAAAGAGAGAAAAGAAGACTAAGAAGAAATATAAGAGCCACC |
| | (5′UTR-011) |
| 338 | GGGAAATAAGAGAGAAAAGAAGAGTAAGAAGATATATAAGAGCCACC |
| | (5′UTR-012) |
| 339 | GGGAAATAAGAGACAAAACAAGAGTAAGAAGAAATATAAGAGCCACC |
| | (5′UTR-013) |
| 340 | GGGAAATTAGAGAGTAAAGAACAGTAAGTAGAATTAAAAGAGCCACC |
| | (5′UTR-014) |
| 341 | GGGAAATAAGAGAGAATAGAAGAGTAAGAAGAAATATAAGAGCCACC |
| | (5′UTR-015) |
| 342 | GGGAAATAAGAGAGAAAAGAAGAGTAAGAAGAAAATTAAGAGCCACC |
| | (5′UTR-016) |
| 343 | GGGAAATAAGAGAGAAAAGAAGAGTAAGAAGAAATTTAAGAGCCACC |
| | (5′UTR-017) |
| 344 | TCAAGCTTTTGGACCCTCGTACAGAAGCTAATACGACTCACTATAGGGAA |
| | ATAAGAGAGAAAAGAAGAGTAAGAAGAAATATAAGAGCCACC |
| | (5′UTR-018) |
| 345 | TGATAATAGTCCATAAAGTAGGAAACACTACAGCTGGAGCCTCGGTGGCC |
| | ATGCTTCTTGCCCCTTGGGCCTCCCCCCAGCCCCTCCTCCCCTTCCTGCACC |
| | CGTACCCCCGTGGTCTTTGAATAAAGTCTGAGTGGGCGGC |
| | (142-3p 5′UTR-001) |
| 346 | TGATAATAGGCTGGAGCCTCGGTGGCTCCATAAAGTAGGAAACACTACAC |
| | ATGCTTCTTGCCCCTTGGGCCTCCCCCCAGCCCCTCCTCCCCTTCCTGCACC |
| | CGTACCCCCGTGGTCTTTGAATAAAGTCTGAGTGGGCGGC |
| | (142-3p 5′UTR-002) |
| 347 | TGATAATAGGCTGGAGCCTCGGTGGCCATGCTTCTTGCCCCTTCCATAAAG |
| | TAGGAAACACTACATGGGCCTCCCCCCAGCCCCTCCTCCCCTTCCTGCACC |
| | CGTACCCCCGTGGTCTTTGAATAAAGTCTGAGTGGGCGGC |
| | (142-3p 5′UTR-003) |
| 348 | TGATAATAGGCTGGAGCCTCGGTGGCCATGCTTCTTGCCCCTTGGGCCTCC |
| | CCCCAGTCCATAAAGTAGGAAACACTACACCCCTCCTCCCCTTCCTGCACC |
| | CGTACCCCCGTGGTCTTTGAATAAAGTCTGAGTGGGCGGC |
| | (142-3p 5′UTR-004) |
| 349 | TGATAATAGGCTGGAGCCTCGGTGGCCATGCTTCTTGCCCCTTGGGCCTCC |
| | CCCCAGCCCCTCCTCCCCTTCTCCATAAAGTAGGAAACACTACACTGCACC |
| | CGTACCCCCGTGGTCTTTGAATAAAGTCTGAGTGGGCGGC |
| | (142-3p 5′UTR-005) |
| 350 | TGATAATAGGCTGGAGCCTCGGTGGCCATGCTTCTTGCCCCTTGGGCCTCC |
| | CCCCAGCCCCTCCTCCCCTTCCTGCACCCGTACCCCCTCCATAAAGTAGGA |
| | AACACTACAGTGGTCTTTGAATAAAGTCTGAGTGGGCGGC |
| | (142-3p 5′UTR-006) |
| 351 | TGATAATAGGCTGGAGCCTCGGTGGCCATGCTTCTTGCCCCTTGGGCCTCC |
| | CCCCAGCCCCTCCTCCCCTTCCTGCACCCGTACCCCCGTGGTCTTTGAATA |
| | AAGTTCCATAAAGTAGGAAACACTACACTGAGTGGGCGGC |
| | (142-3p 5′UTR-007) |
| 352 | GCGCCTGCCCACCTGCCACCGACTGCTGGAACCCAGCCAGTGGGAGGGCC |
| | TGGCCCACCAGAGTCCTGCTCCCTCACTCCTCGCCCCGCCCCCTGTCCCAG |
| | AGTCCCACCTGGGGGCTCTCTCCACCCTTCTCAGAGTTCCAGTTTCAACCA |
| | GAGTTCCAACCAATGGGCTCCATCCTCTGGATTCTGGCCAATGAAATATCT |
| | CCCTGGCAGGGTCCTCTTCTTTTCCCAGAGCTCCACCCCAACCAGGAGCTC |
| | TAGTTAATGGAGAGCTCCCAGCACACTCGGAGCTTGTGCTTTGTCTCCACG |
| | CAAAGCGATAAATAAAAGCATTGGTGGCCTTTGGTCTTTGAATAAAGCCT |
| | GAGTAGGAAGTCTAGA (3′UTR-001) |
| 353 | GCCCCTGCCGCTCCCACCCCCACCCATCTGGGCCCCGGGTTCAAGAGAGA |
| | GCGGGGTCTGATCTCGTGTAGCCATATAGAGTTTGCTTCTGAGTGTCTGCT |
| | TTGTTTAGTAGAGGTGGGCAGGAGGAGCTGAGGGGCTGGGGCTGGGGTG |
| | TTGAAGTTGGCTTTGCATGCCCAGCGATGCGCCTCCCTGTGGGATGTCATC |
| | ACCCTGGGAACCGGGAGTGGCCCTTGGCTCACTGTGTTCTGCATGGTTTG |
| | GATCTGAATTAATTGTCCTTTCTTCTAAATCCCAACCGAACTTCTTCCAAC |
| | CTCCAAACTGGCTGTAACCCCAAATCCAAGCCATTAACTACACCTGACAG |
| | TAGCAATTGTCTGATTAATCACTGGCCCCTTGAAGACAGCAGAATGTCCC |
| | TTTGCAATGAGGAGGAGATCTGGGCTGGGCGGGCCAGCTGGGGAAGCAT |
| | TTGACTATCTGGAACTTGTGTGTGCCTCCTCAGGTATGGCAGTGACTCACC |
| | TGGTTTTAATAAAACAACCTGCAACATCTCATGGTCTTTGAATAAAGCCTG |
| | AGTAGGAAGTCTAGA (3′UTR-002) |
| 354 | ACACACTCCACCTCCAGCACGCGACTTCTCAGGACGACGAATCTTCTCAA |
| | TGGGGGGGCGGCTGAGCTCCAGCCACCCCGCAGTCACTTTCTTTGTAACA |
| | ACTTCCGTTGCTGCCATCGTAAACTGACACAGTGTTTATAACGTGTACATA |
| | CATTAACTTATTACCTCATTTTGTTATTTTTCGAAACAAAGCCCTGTGGAA |
| | GAAAATGGAAAACTTGAAGAAGCATTAAAGTCATTCTGTTAAGCTGCGTA |
| | AATGGTCTTTGAATAAAGCCTGAGTAGGAAGTCTAGA (3′UTR-003) |
| 355 | CATCACATTTAAAAGCATCTCAGCCTACCATGAGAATAAGAGAAAGAAA |
| | ATGAAGATCAAAAGCTTATTCATCTGTTTTTCTTTTTCGTTGGTGTAAAGC |
| | CAACACCCTGTCTAAAAAACATAAATTTCTTTAATCATTTTGCCTCTTTTCT |
| | CTGTGCTTCAATTAATAAAAAATGGAAAGAATCTAATAGAGTGGTACAGC |
| | ACTGTTATTTTTCAAAGATGTGTTGCTATCCTGAAAATTCTGTAGGTTCTG |
| | TGGAAGTTCCAGTGTTCTCTCTTATTCCACTTCGGTAGAGGATTTCTAGTT |
| | TCTTGTGGGCTAATTAAATAAATCATTAATACTCTTCTAATGGTCTTTGAA |
| | TAAAGCCTGAGTAGGAAGTCTAGA (3′UTR-004) |
| 356 | GCTGCCTTCTGCGGGGCTTGCCTTCTGGCCATGCCCTTCTTCTCTCCCTTGC |
| | ACCTGTACCTCTTGGTCTTTGAATAAAGCCTGAGTAGGAAGGCGGCCGCT |
| | CGAGCATGCATCTAGA (3′UTR-005) |
| 357 | GCCAAGCCCTCCCCATCCCATGTATTTATCTCTATTTAATATTTATGTCTAT |
| | TTAAGCCTCATATTTAAAGACAGGGAAGAGCAGAACGGAGCCCCAGGCC |
| | TCTGTGTCCTTCCCTGCATTTCTGAGTTTCATTCTCCTGCCTGTAGCAGTGA |
| | GAAAAAGCTCCTGTCCTCCCATCCCCTGGACTGGGAGGTAGATAGGTAAA |
| | TACCAAGTATTTATTACTATGACTGCTCCCCAGCCCTGGCTCTGCAATGGG |
| | CACTGGGATGAGCCGCTGTGAGCCCCTGGTCCTGAGGGTCCCCACCTGGG |
| | ACCCTTGAGAGTATCAGGTCTCCCACGTGGGAGACAAGAAATCCCTGTTT |
| | AATATTTAAACAGCAGTGTTCCCCATCTGGGTCCTTGCACCCCTCACTCTG |
| | GCCTCAGCCGACTGCACAGCGGCCCCTGCATCCCCTTGGCTGTGAGGCCC |
| | CTGGACAAGCAGAGGTGGCCAGAGCTGGGAGGCATGGCCCTGGGGTCCC |
| | ACGAATTTGCTGGGGAATCTCGTTTTTCTTCTTAAGACTTTTGGGACATGG |
| | TTTGACTCCCGAACATCACCGACGCGTCTCCTGTTTTTCTGGGTGGCCTCG |
| | GGACACCTGCCCTGCCCCCACGAGGGTCAGGACTGTGACTCTTTTTAGGG |
| | CCAGGCAGGTGCCTGGACATTTGCCTTGCTGGACGGGGACTGGGGATGTG |
| | GGAGGGAGCAGACAGGAGGAATCATGTCAGGCCTGTGTGTGAAAGGAAG |
| | CTCCACTGTCACCCTCCACCTCTTCACCCCCCACTCACCAGTGTCCCCTCC |
| | ACTGTCACATTGTAACTGAACTTCAGGATAATAAAGTGTTTGCCTCCATGG |
| | TCTTTGAATAAAGCCTGAGTAGGAAGGCGGCCGCTCGAGCATGCATCTAG |
| | A (3′UTR-006) |
| 358 | ACTCAATCTAAATTAAAAAAGAAAGAAATTTGAAAAAACTTTCTCTTTGC |
| | CATTTCTTCTTCTTCTTTTTTAACTGAAAGCTGAATCCTTCCATTTCTTCTG |
| | CACATCTACTTGCTTAAATTGTGGGCAAAAGAGAAAAAGAAGGATTGATC |
| | AGAGCATTGTGCAATACAGTTTCATTAACTCCTTCCCCCGCTCCCCCAAAA |
| | ATTTGAATTTTTTTTTCAACACTCTTACACCTGTTATGGAAAATGTCAACC |
| | TTTGTAAGAAAACCAAAATAAAAATTGAAAAATAAAAACCATAAACATTT |
| | GCACCACTTGTGGCTTTTGAATATCTTCCACAGAGGGAAGTTTAAAACCC |
| | AAACTTCCAAAGGTTTAAACTACCTCAAAACACTTTCCCATGAGTGTGAT |
| | CCACATTGTTAGGTGCTGACCTAGACAGAGATGAACTGAGGTCCTTGTTTT |
| | GTTTTGTTCATAATACAAAGGTGCTAATTAATAGTATTTCAGATACTTGAA |
| | GAATGTTGATGGTGCTAGAAGAATTTGAGAAGAAATACTCCTGTATTGAG |
| | TTGTATCGTGTGGTGTATTTTTTAAAAAATTTGATTTAGCATTCATATTTTC |
| | CATCTTATTCCCAATTAAAAGTATGCAGATTATTTGCCCAAATCTTCTTCA |
| | GATTCAGCATTTGTTCTTTGCCAGTCTCATTTTCATCTTCTTCCATGGTTCC |
| | ACAGAAGCTTTGTTTCTTGGGCAAGCAGAAAAATTAAATTGTACCTATTTT |
| | GTATATGTGAGATGTTTAAATAAATTGTGAAAAAAATGAAATAAAGCATG |
| | TTTGGTTTTCCAAAAGAACATAT (3′UTR-007) |
| 359 | CGCCGCCGCCCGGGCCCCGCAGTCGAGGGTCGTGAGCCCACCCCGTCCAT |
| | GGTGCTAAGCGGGCCCGGGTCCCACACGGCCAGCACCGCTGCTCACTCGG |
| | ACGACGCCCTGGGCCTGCACCTCTCCAGCTCCTCCCACGGGGTCCCCGTA |
| | GCCCCGGCCCCCGCCCAGCCCCAGGTCTCCCCAGGCCCTCCGCAGGCTGC |
| | CCGGCCTCCCTCCCCCTGCAGCCATCCCAAGGCTCCTGACCTACCTGGCCC |
| | CTGAGCTCTGGAGCAAGCCCTGACCCAATAAAGGCTTTGAACCCAT |
| | (3′UTR-008) |
| 360 | GGGGCTAGAGCCCTCTCCGCACAGCGTGGAGACGGGGCAAGGAGGGGGG |
| | TTATTAGGATTGGTGGTTTTGTTTTGCTTTGTTTAAAGCCGTGGGAAAATG |
| | GCACAACTTTACCTCTGTGGGAGATGCAACACTGAGAGCCAAGGGGTGGG |
| | AGTTGGGATAATTTTTATATAAAAGAAGTTTTTCCACTTTGAATTGCTAAA |
| | AGTGGCATTTTTCCTATGTGCAGTCACTCCTCTCATTTCTAAAATAGGGAC |
| | GTGGCCAGGCACGGTGGCTCATGCCTGTAATCCCAGCACTTTGGGAGGCC |
| | GAGGCAGGCGGCTCACGAGGTCAGGAGATCGAGACTATCCTGGCTAACA |
| | CGGTAAAACCCTGTCTCTACTAAAAGTACAAAAAATTAGCTGGGCGTGGT |
| | GGTGGGCACCTGTAGTCCCAGCTACTCGGGAGGCTGAGGCAGGAGAAAG |
| | GCATGAATCCAAGAGGCAGAGCTTGCAGTGAGCTGAGATCACGCCATTGC |
| | ACTCCAGCCTGGGCAACAGTGTTAAGACTCTGTCTCAAATATAAATAAAT |
| | AAATAAATAAATAAATAAATAAATAAAAATAAAGCGAGATGTTGCCCTC |
| | AAA (3′UTR-009) |
| 361 | GGCCCTGCCCCGTCGGACTGCCCCCAGAAAGCCTCCTGCCCCCTGCCAGT |
| | GAAGTCCTTCAGTGAGCCCCTCCCCAGCCAGCCCTTCCCTGGCCCCGCCG |
| | GATGTATAAATGTAAAAATGAAGGAATTACATTTTATATGTGAGCGAGCA |
| | AGCCGGCAAGCGAGCACAGTATTATTTCTCCATCCCCTCCCTGCCTGCTCC |
| | TTGGCACCCCCATGCTGCCTTCAGGGAGACAGGCAGGGAGGGCTTGGGGC |
| | TGCACCTCCTACCCTCCCACCAGAACGCACCCCACTGGGAGAGCTGGTGG |
| | TGCAGCCTTCCCCTCCCTGTATAAGACACTTTGCCAAGGCTCTCCCCTCTC |
| | GCCCCATCCCTGCTTGCCCGCTCCCACAGCTTCCTGAGGGCTAATTCTGGG |
| | AAGGGAGAGTTCTTTGCTGCCCCTGTCTGGAAGACGTGGCTCTGGGTGAG |
| | GTAGGCGGGAAAGGATGGAGTGTTTTAGTTCTTGGGGGAGGCCACCCCAA |
| | ACCCCAGCCCCAACTCCAGGGGCACCTATGAGATGGCCATGCTCAACCCC |
| | CCTCCCAGACAGGCCCTCCCTGTCTCCAGGGCCCCCACCGAGGTTCCCAG |
| | GGCTGGAGACTTCCTCTGGTAAACATTCCTCCAGCCTCCCCTCCCCTGGGG |
| | ACGCCAAGGAGGTGGGCCACACCCAGGAAGGGAAAGCGGGCAGCCCCGT |
| | TTTGGGGACGTGAACGTTTTAATAATTTTTGCTGAATTCCTTTACAACTAA |
| | ATAACACAGATATTGTTATAAATAAAATTGT (3′UTR-010) |
| 362 | ATATTAAGGATCAAGCTGTTAGCTAATAATGCCACCTCTGCAGTTTTGGG |
| | AACAGGCAAATAAAGTATCAGTATACATGGTGATGTACATCTGTAGCAAA |
| | GCTCTTGGAGAAAATGAAGACTGAAGAAAGCAAAGCAAAAACTGTATAG |
| | AGAGATTTTTCAAAAGCAGTAATCCCTCAATTTTAAAAAAGGATTGAAAA |
| | TTCTAAATGTCTTTCTGTGCATATTTTTTGTGTTAGGAATCAAAAGTATTTT |
| | ATAAAAGGAGAAAGAACAGCCTCATTTTAGATGTAGTCCTGTTGGATTTT |
| | TTATGCCTCCTCAGTAACCAGAAATGTTTTAAAAAACTAAGTGTTTAGGAT |
| | TTCAAGACAACATTATACATGGCTCTGAAATATCTGACACAATGTAAACA |
| | TTGCAGGCACCTGCATTTTATGTTTTTTTTTTCAACAAATGTGACTAATTTG |
| | AAACTTTTATGAACTTCTGAGCTGTCCCCTTGCAATTCAACCGCAGTTTGA |
| | ATTAATCATATCAAATCAGTTTTAATTTTTTAAATTGTACTTCAGAGTCTA |
| | TATTTCAAGGGCACATTTTCTCACTACTATTTTAATACATTAAAGGACTAA |
| | ATAATCTTTCAGAGATGCTGGAAACAAATCATTTGCTTTATATGTTTCATT |
| | AGAATACCAATGAAACATACAACTTGAAAATTAGTAATAGTATTTTTGAA |
| | GATCCCATTTCTAATTGGAGATCTCTTTAATTTCGATCAACTTATAATGTG |
| | TAGTACTATATTAAGTGCACTTGAGTGGAATTCAACATTTGACTAATAAA |
| | ATGAGTTCATCATGTTGGCAAGTGATGTGGCAATTATCTCTGGTGACAAA |
| | AGAGTAAAATCAAATATTTCTGCCTGTTACAAATATCAAGGAAGACCTGC |
| | TACTATGAAATAGATGACATTAATCTGTCTTCACTGTTTATAATACGGATG |
| | GATTTTTTTTCAAATCAGTGTGTGTTTTGAGGTCTTATGTAATTGATGACA |
| | TTTGAGAGAAATGGTGGCTTTTTTTAGCTACCTCTTTGTTCATTTAAGCAC |
| | CAGTAAAGATCATGTCTTTTTATAGAAGTGTAGATTTTCTTTGTGACTTTG |
| | CTATCGTGCCTAAAGCTCTAAATATAGGTGAATGTGTGATGAATACTCAG |
| | ATTATTTGTCTCTCTATATAATTAGTTTGGTACTAAGTTTCTCAAAAAATT |
| | ATTAACACATGAAAGACAATCTCTAAACCAGAAAAAGAAGTAGTACAAA |
| | TTTTGTTACTGTAATGCTCGCGTTTAGTGAGTTTAAAACACACAGTATCTT |
| | TTGGTTTTATAATCAGTTTCTATTTTGCTGTGCCTGAGATTAAGATCTGTGT |
| | ATGTGTGTGTGTGTGTGTGTGCGTTTGTGTGTTAAAGCAGAAAAGACTTTT |
| | TTAAAAGTTTTAAGTGATAAATGCAATTTGTTAATTGATCTTAGATCACTA |
| | GTAAACTCAGGGCTGAATTATACCATGTATATTCTATTAGAAGAAAGTAA |
| | ACACCATCTTTATTCCTGCCCTTTTTCTTCTCTCAAAGTAGTTGTAGTTATA |
| | TCTAGAAAGAAGCAATTTTGATTTCTTGAAAAGGTAGTTCCTGCACTCAGT |
| | TTAAACTAAAAATAATCATACTTGGATTTTATTTATTTTTGTCATAGTAAA |
| | AATTTTAATTTATATATATTTTTATTTAGTATTATCTTATTCTTTGCTATTTG |
| | CCAATCCTTTGTCATCAATTGTGTTAAATGAATTGAAAATTCATGCCCTGT |
| | TCATTTTATTTTACTTTATTGGTTAGGATATTTAAAGGATTTTTGTATATAT |
| | AATTTCTTAAATTAATATTCCAAAAGGTTAGTGGACTTAGATTATAAATTA |
| | TGGCAAAAATCTAAAAACAACAAAAATGATTTTTATACATTCTATTTCATT |
| | ATTCCTCTTTTTCCAATAAGTCATACAATTGGTAGATATGACTTATTTTATT |
| | TTTGTATTATTCACTATATCTTTATGATATTTAAGTATAAATAATTAAAAA |
| | AATTTATTGTACCTTATAGTCTGTCACCAAAAAAAAAAAATTATCTGTAG |
| | GTAGTGAAATGCTAATGTTGATTTGTCTTTAAGGGCTTGTTAACTATCCTT |
| | TATTTTCTCATTTGTCTTAAATTAGGAGTTTGTGTTTAAATTACTCATCTAA |
| | GCAAAAAATGTATATAAATCCCATTACTGGGTATATACCCAAAGGATTAT |
| | AAATCATGCTGCTATAAAGACACATGCACACGTATGTTTATTGCAGCACT |
| | ATTCACAATAGCAAAGACTTGGAACCAACCCAAATGTCCATCAATGATAG |
| | ACTTGATTAAGAAAATGTGCACATATACACCATGGAATACTATGCAGCCA |
| | TAAAAAAGGATGAGTTCATGTCCTTTGTAGGGACATGGATAAAGCTGGAA |
| | ACCATCATTCTGAGCAAACTATTGCAAGGACAGAAAACCAAACACTGCAT |
| | GTTCTCACTCATAGGTGGGAATTGAACAATGAGAACACTTGGACACAAGG |
| | TGGGGAACACCACACACCAGGGCCTGTCATGGGGTGGGGGGAGTGGGGA |
| | GGGATAGCATTAGGAGATATACCTAATGTAAATGATGAGTTAATGGGTGC |
| | AGCACACCAACATGGCACATGTATACATATGTAGCAAACCTGCACGTTGT |
| | GCACATGTACCCTAGAACTTAAAGTATAATTAAAAAAAAAAAGAAAACA |
| | GAAGCTATTTATAAAGAAGTTATTTGCTGAAATAAATGTGATCTTTCCCAT |
| | TAAAAAAATAAAGAAATTTTGGGGTAAAAAAACACAATATATTGTATTCT |
| | TGAAAAATTCTAAGAGAGTGGATGTGAAGTGTTCTCACCACAAAAGTGAT |
| | AACTAATTGAGGTAATGCACATATTAATTAGAAAGATTTTGTCATTCCAC |
| | AATGTATATATACTTAAAAATATGTTATACACAATAAATACATACATTAA |
| | AAAATAAGTAAATGTA (3′UTR-011) |
| 363 | CCCACCCTGCACGCCGGCACCAAACCCTGTCCTCCCACCCCTCCCCACTCA |
| | TCACTAAACAGAGTAAAATGTGATGCGAATTTTCCCGACCAACCTGATTC |
| | GCTAGATTTTTTTTAAGGAAAAGCTTGGAAAGCCAGGACACAACGCTGCT |
| | GCCTGCTTTGTGCAGGGTCCTCCGGGGCTCAGCCCTGAGTTGGCATCACCT |
| | GCGCAGGGCCCTCTGGGGCTCAGCCCTGAGCTAGTGTCACCTGCACAGGG |
| | CCCTCTGAGGCTCAGCCCTGAGCTGGCGTCACCTGTGCAGGGCCCTCTGG |
| | GGCTCAGCCCTGAGCTGGCCTCACCTGGGTTCCCCACCCCGGGCTCTCCTG |
| | CCCTGCCCTCCTGCCCGCCCTCCCTCCTGCCTGCGCAGCTCCTTCCCTAGG |
| | CACCTCTGTGCTGCATCCCACCAGCCTGAGCAAGACGCCCTCTCGGGGCC |
| | TGTGCCGCACTAGCCTCCCTCTCCTCTGTCCCCATAGCTGGTTTTTCCCACC |
| | AATCCTCACCTAACAGTTACTTTACAATTAAACTCAAAGCAAGCTCTTCTC |
| | CTCAGCTTGGGGCAGCCATTGGCCTCTGTCTCGTTTTGGGAAACCAAGGTC |
| | AGGAGGCCGTTGCAGACATAAATCTCGGCGACTCGGCCCCGTCTCCTGAG |
| | GGTCCTGCTGGTGACCGGCCTGGACCTTGGCCCTACAGCCCTGGAGGCCG |
| | CTGCTGACCAGCACTGACCCCGACCTCAGAGAGTACTCGCAGGGGCGCTG |
| | GCTGCACTCAAGACCCTCGAGATTAACGGTGCTAACCCCGTCTGCTCCTCC |
| | CTCCCGCAGAGACTGGGGCCTGGACTGGACATGAGAGCCCCTTGGTGCCA |
| | CAGAGGGCTGTGTCTTACTAGAAACAACGCAAACCTCTCCTTCCTCAGAA |
| | TAGTGATGTGTTCGACGTTTTATCAAAGGCCCCCTTTCTATGTTCATGTTA |
| | GTTTTGCTCCTTCTGTGTTTTTTTCTGAACCATATCCATGTTGCTGACTTTT |
| | CCAAATAAAGGTTTTCACTCCTCTC (3′UTR-012) |
| 364 | AGAGGCCTGCCTCCAGGGCTGGACTGAGGCCTGAGCGCTCCTGCCGCAGA |
| | GCTGGCCGCGCCAAATAATGTCTCTGTGAGACTCGAGAACTTTCATTTTTT |
| | TCCAGGCTGGTTCGGATTTGGGGTGGATTTTGGTTTTGTTCCCCTCCTCCA |
| | CTCTCCCCCACCCCCTCCCCGCCCTTTTTTTTTTTTTTTTTTAAACTGGTATT |
| | TTATCTTTGATTCTCCTTCAGCCCTCACCCCTGGTTCTCATCTTTCTTGATC |
| | AACATCTTTTCTTGCCTCTGTCCCCTTCTCTCATCTCTTAGCTCCCCTCCAA |
| | CCTGGGGGGCAGTGGTGTGGAGAAGCCACAGGCCTGAGATTTCATCTGCT |
| | CTCCTTCCTGGAGCCCAGAGGAGGGCAGCAGAAGGGGGTGGTGTCTCCAA |
| | CCCCCCAGCACTGAGGAAGAACGGGGCTCTTCTCATTTCACCCCTCCCTTT |
| | CTCCCCTGCCCCCAGGACTGGGCCACTTCTGGGTGGGGCAGTGGGTCCCA |
| | GATTGGCTCACACTGAGAATGTAAGAACTACAAACAAAATTTCTATTAAA |
| | TTAAATTTTGTGTCTCC (3′UTR-013) |
| 365 | CTCCCTCCATCCCAACCTGGCTCCCTCCCACCCAACCAACTTTCCCCCCAA |
| | CCCGGAAACAGACAAGCAACCCAAACTGAACCCCCTCAAAAGCCAAAAA |
| | ATGGGAGACAATTTCACATGGACTTTGGAAAATATTTTTTTCCTTTGCATT |
| | CATCTCTCAAACTTAGTTTTTATCTTTGACCAACCGAACATGACCAAAAAC |
| | CAAAAGTGCATTCAACCTTACCAAAAAAAAAAAAAAAAAAAGAATAAAT |
| | AAATAACTTTTTAAAAAAGGAAGCTTGGTCCACTTGCTTGAAGACCCATG |
| | CGGGGGTAAGTCCCTTTCTGCCCGTTGGGCTTATGAAACCCCAATGCTGCC |
| | CTTTCTGCTCCTTTCTCCACACCCCCCTTGGGGCCTCCCCTCCACTCCTTCC |
| | CAAATCTGTCTCCCCAGAAGACACAGGAAACAATGTATTGTCTGCCCAGC |
| | AATCAAAGGCAATGCTCAAACACCCAAGTGGCCCCCACCCTCAGCCCGCT |
| | CCTGCCCGCCCAGCACCCCCAGGCCCTGGGGGACCTGGGGTTCTCAGACT |
| | GCCAAAGAAGCCTTGCCATCTGGCGCTCCCATGGCTCTTGCAACATCTCCC |
| | CTTCGTTTTTGAGGGGGTCATGCCGGGGGAGCCACCAGCCCCTCACTGGG |
| | TTCGGAGGAGAGTCAGGAAGGGCCACGACAAAGCAGAAACATCGGATTT |
| | GGGGAACGCGTGTCAATCCCTTGTGCCGCAGGGCTGGGCGGGAGAGACT |
| | GTTCTGTTCCTTGTGTAACTGTGTTGCTGAAAGACTACCTCGTTCTTGTCTT |
| | GATGTGTCACCGGGGCAACTGCCTGGGGGCGGGGATGGGGGCAGGGTGG |
| | AAGCGGCTCCCCATTTTATACCAAAGGTGCTACATCTATGTGATGGGTGG |
| | GGTGGGGAGGGAATCACTGGTGCTATAGAAATTGAGATGCCCCCCCAGGC |
| | CAGCAAATGTTCCTTTTTGTTCAAAGTCTATTTTTATTCCTTGATATTTTTC |
| | TTTTTTTTTTTTTTTTTTTGTGGATGGGGACTTGTGAATTTTTCTAAAGGTG |
| | CTATTTAACATGGGAGGAGAGCGTGTGCGGCTCCAGCCCAGCCCGCTGCT |
| | CACTTTCCACCCTCTCTCCACCTGCCTCTGGCTTCTCAGGCCTCTGCTCTCC |
| | GACCTCTCTCCTCTGAAACCCTCCTCCACAGCTGCAGCCCATCCTCCCGGC |
| | TCCCTCCTAGTCTGTCCTGCGTCCTCTGTCCCCGGGTTTCAGAGACAACTT |
| | CCCAAAGCACAAAGCAGTTTTTCCCCCTAGGGGTGGGAGGAAGCAAAAG |
| | ACTCTGTACCTATTTTGTATGTGTATAATAATTTGAGATGTTTTTAATTATT |
| | TTGATTGCTGGAATAAAGCATGTGGAAATGACCCAAACATAATCCGCAGT |
| | GGCCTCCTAATTTCCTTCTTTGGAGTTGGGGGAGGGGTAGACATGGGGAA |
| | GGGGCTTTGGGGTGATGGGCTTGCCTTCCATTCCTGCCCTTTCCCTCCCCA |
| | CTATTCTCTTCTAGATCCCTCCATAACCCCACTCCCCTTTCTCTCACCCTTC |
| | TTATACCGCAAACCTTTCTACTTCCTCTTTCATTTTCTATTCTTGCAATTTC |
| | CTTGCACCTTTTCCAAATCCTCTTCTCCCCTGCAATACCATACAGGCAATC |
| | CACGTGCACAACACACACACACACTCTTCACATCTGGGGTTGTCCAAACC |
| | TCATACCCACTCCCCTTCAAGCCCATCCACTCTCCACCCCCTGGATGCCCT |
| | GCACTTGGTGGCGGTGGGATGCTCATGGATACTGGGAGGGTGAGGGGAG |
| | TGGAACCCGTGAGGAGGACCTGGGGGCCTCTCCTTGAACTGACATGAAGG |
| | GTCATCTGGCCTCTGCTCCCTTCTCACCCACGCTGACCTCCTGCCGAAGGA |
| | GCAACGCAACAGGAGAGGGGTCTGCTGAGCCTGGCGAGGGTCTGGGAGG |
| | GACCAGGAGGAAGGCGTGCTCCCTGCTCGCTGTCCTGGCCCTGGGGGAGT |
| | GAGGGAGACAGACACCTGGGAGAGCTGTGGGGAAGGCACTCGCACCGTG |
| | CTCTTGGGAAGGAAGGAGACCTGGCCCTGCTCACCACGGACTGGGTGCCT |
| | CGACCTCCTGAATCCCCAGAACACAACCCCCCTGGGCTGGGGTGGTCTGG |
| | GGAACCATCGTGCCCCCGCCTCCCGCCTACTCCTTTTTAAGCTT |
| | (3′UTR-014) |
| 366 | TTGGCCAGGCCTGACCCTCTTGGACCTTTCTTCTTTGCCGACAACCACTGC |
| | CCAGCAGCCTCTGGGACCTCGGGGTCCCAGGGAACCCAGTCCAGCCTCCT |
| | GGCTGTTGACTTCCCATTGCTCTTGGAGCCACCAATCAAAGAGATTCAAA |
| | GAGATTCCTGCAGGCCAGAGGCGGAACACACCTTTATGGCTGGGGCTCTC |
| | CGTGGTGTTCTGGACCCAGCCCCTGGAGACACCATTCACTTTTACTGCTTT |
| | GTAGTGACTCGTGCTCTCCAACCTGTCTTCCTGAAAAACCAAGGCCCCCTT |
| | CCCCCACCTCTTCCATGGGGTGAGACTTGAGCAGAACAGGGGCTTCCCCA |
| | AGTTGCCCAGAAAGACTGTCTGGGTGAGAAGCCATGGCCAGAGCTTCTCC |
| | CAGGCACAGGTGTTGCACCAGGGACTTCTGCTTCAAGTTTTGGGGTAAAG |
| | ACACCTGGATCAGACTCCAAGGGCTGCCCTGAGTCTGGGACTTCTGCCTC |
| | CATGGCTGGTCATGAGAGCAAACCGTAGTCCCCTGGAGACAGCGACTCCA |
| | GAGAACCTCTTGGGAGACAGAAGAGGCATCTGTGCACAGCTCGATCTTCT |
| | ACTTGCCTGTGGGGAGGGGAGTGACAGGTCCACACACCACACTGGGTCAC |
| | CCTGTCCTGGATGCCTCTGAAGAGAGGGACAGACCGTCAGAAACTGGAG |
| | AGTTTCTATTAAAGGTCATTTAAACCA (3′UTR-015) |
| 367 | TCCTCCGGGACCCCAGCCCTCAGGATTCCTGATGCTCCAAGGCGACTGAT |
| | GGGCGCTGGATGAAGTGGCACAGTCAGCTTCCCTGGGGGCTGGTGTCATG |
| | TTGGGCTCCTGGGGCGGGGGCACGGCCTGGCATTTCACGCATTGCTGCCA |
| | CCCCAGGTCCACCTGTCTCCACTTTCACAGCCTCCAAGTCTGTGGCTCTTC |
| | CCTTCTGTCCTCCGAGGGGCTTGCCTTCTCTCGTGTCCAGTGAGGTGCTCA |
| | GTGATCGGCTTAACTTAGAGAAGCCCGCCCCCTCCCCTTCTCCGTCTGTCC |
| | CAAGAGGGTCTGCTCTGAGCCTGCGTTCCTAGGTGGCTCGGCCTCAGCTG |
| | CCTGGGTTGTGGCCGCCCTAGCATCCTGTATGCCCACAGCTACTGGAATCC |
| | CCGCTGCTGCTCCGGGCCAAGCTTCTGGTTGATTAATGAGGGCATGGGGT |
| | GGTCCCTCAAGACCTTCCCCTACCTTTTGTGGAACCAGTGATGCCTCAAAG |
| | ACAGTGTCCCCTCCACAGCTGGGTGCCAGGGGCAGGGGATCCTCAGTATA |
| | GCCGGTGAACCCTGATACCAGGAGCCTGGGCCTCCCTGAACCCCTGGCTT |
| | CCAGCCATCTCATCGCCAGCCTCCTCCTGGACCTCTTGGCCCCCAGCCCCT |
| | TCCCCACACAGCCCCAGAAGGGTCCCAGAGCTGACCCCACTCCAGGACCT |
| | AGGCCCAGCCCCTCAGCCTCATCTGGAGCCCCTGAAGACCAGTCCCACCC |
| | ACCTTTCTGGCCTCATCTGACACTGCTCCGCATCCTGCTGTGTGTCCTGTTC |
| | CATGTTCCGGTTCCATCCAAATACACTTTCTGGAACAAA (3′UTR-016) |
| 368 | GCTGGAGCCTCGGTGGCCATGCTTCTTGCCCCTTGGGCCTCCCCCCAGCCC |
| | CTCCTCCCCTTCCTGCACCCGTACCCCCGTGGTCTTTGAATAAAGTCTGAG |
| | TGGGCGGC (3′UTR-017) |
| 369 | TGATAATAGGCTGGAGCCTCGGTGGCCATGCTTCTTGCCCCTTGGGCCTCC |
| | CCCCAGCCCCTCCTCCCCTTCCTGCACCCGTACCCCCGTGGTCTTTGAATA |
| | AAGTCTGAGTGGGCGGC (3′UTR-018) |
| 370 | MEEQWAREIGAQLRRMADDLNAQYERRGSGVKQTLNFDLLKLAGDVESNP |
| | GPEEQWAREIGAQLRRMADDLNAQYERRGSGVKQTLNFDLLKLAGDVESNP |
| | GPEEQWAREIGAQLRRMADDLNAQYERRGKPIPNPLLGLDST |
| | (Puma BH3 (x3P2A).v5 |
| 371 | ATGGAGGAGCAATGGGCTAGAGAGATCGGCGCACAGCTGCGGCGCATGGC |
| | CGATGATCTGAACGCCCAATACGAGAGGAGAGGTTCCGGAGTGAAGCAGA |
| | CTCTGAACTTCGATCTGCTCAAGCTTGCGGGCGACGTGGAATCGAACCCCG |
| | GCCCTGAGGAACAATGGGCGCGCGAAATCGGTGCCCAGCTCCGCCGGATG |
| | GCAGACGACCTGAACGCGCAGTACGAGCGGCGGGGGAGCGGGGTCAAGC |
| | AGACCCTGAATTTCGACCTTCTGAAGCTGGCCGGAGATGTGGAGTCAAAC |
| | CCGGGACCCGAAGAACAGTGGGCCAGGGAAATTGGAGCTCAGCTGCGGA |
| | GAATGGCCGACGACCTCAACGCCCAGTACGAACGGCGCGGAAAACCTATC |
| | CCGAACCCACTCTTGGGCCTGGACTCCACC (Puma BH3 (x3P2A).v5 |
| 372 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGATGG |
| | AAGGAATCAGCAACTTCAAGACCCCATCCAAGCTGTCCGAGAAGAAAAA |
| | GGGTTCCGGAGTGAAGCAGACCCTGAACTTCGATCTGCTCAAGCTCGCCG |
| | GGGACGTGGAAAGCAACCCTGGTCCCATGGAGGGCATCTCGAACTTTAAG |
| | ACCCCCTCGAAGCTTTCGGAGAAGAAGAAGGGATCCGGCGTCAAGCAGA |
| | CTCTGAATTTCGACTTGCTGAAGCTCGCGGGCGATGTGGAATCAAACCCG |
| | GGGCCTATGGAAGGCATCTCCAACTTCAAAACTCCGTCCAAGCTGAGCGA |
| | GAAAAAGAAGGGAAAGCCCATTCCGAACCCTCTGCTGGGACTGGACAGC |
| | ACCTGATAATAGGCTGGAGCCTCGGTGGCCATGCTTCTTGCCCCTTGGGCC |
| | TCCCCCCAGCCCCTCCTCCCCTTCCTGCACCCGTACCCCCGTGGTCTTTGA |
| | ATAAAGTCTGAGTGGGCGGC |
| | (TandemPep.TOPK_codon optimized (5′ UTR, ORF, 3′ UTR) |
| 373 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGATGG |
| | AAGGCATCAGCAACTTCAAGACCCCAAGCAAGCTGAGCGAGAAGAAGAA |
| | GGGCTCCGGCGTGAAGCAGACCTTGAACTTCGACCTGCTCAAACTTGCCG |
| | GCGACGTGGAGAGCAACCCCGGCCCCATGGAGGGGATCAGTAACTTCAA |
| | GACCCCCAGCAAGCTGAGCGAGAAGAAGAAGGGTAGCGGCGTGAAACAG |
| | ACCCTGAATTTCGATCTGCTGAAGCTGGCCGGCGACGTGGAGAGCAACCC |
| | CGGCCCCATGGAGGGCATAAGCAATTTCAAGACCCCCAGCAAGCTGAGC |
| | GAAAAGAAAAAGGGCAAGCCCATTCCCAACCCCCTTCTGGGCCTTGACAG |
| | CACCTGATAATAGGCTGGAGCCTCGGTGGCCATGCTTCTTGCCCCTTGGGC |
| | CTCCCCCCAGCCCCTCCTCCCCTTCCTGCACCCGTACCCCCGTGGTCTTTG |
| | AATAAAGTCTGAGTGGGCGGC |
| | (TandemPep.TOPK (5′ UTR, ORF, 3′ UTR)) |
| 374 | ATGATGGAAGGAATCAGCAACTTCAAGACCCCATCCAAGCTGTCCGAGAA |
| | GAAAAAGGGTTCCGGAGTGAAGCAGACCCTGAACTTCGATCTGCTCAAGC |
| | TCGCCGGGGACGTGGAAAGCAACCCTGGTCCCATGGAGGGCATCTCGAAC |
| | TTTAAGACCCCCTCGAAGCTTTCGGAGAAGAAGAAGGGATCCGGCGTCAA |
| | GCAGACTCTGAATTTCGACTTGCTGAAGCTCGCGGGCGATGTGGAATCAA |
| | ACCCGGGGCCTATGGAAGGCATCTCCAACTTCAAAACTCCGTCCAAGCTG |
| | AGCGAGAAAAAGAAGGGAAAGCCCATTCCGAACCCTCTGCTGGGACTGG |
| | ACAGCACC (TandemPep.TOPK codon optimized (ORF)) |
| 375 | ATGATGGAAGGCATCAGCAACTTCAAGACCCCAAGCAAGCTGAGCGAGA |
| | AGAAGAAGGGCTCCGGCGTGAAGCAGACCTTGAACTTCGACCTGCTCAAA |
| | CTTGCCGGCGACGTGGAGAGCAACCCCGGCCCCATGGAGGGGATCAGTA |
| | ACTTCAAGACCCCCAGCAAGCTGAGCGAGAAGAAGAAGGGTAGCGGCGT |
| | GAAACAGACCCTGAATTTCGATCTGCTGAAGCTGGCCGGCGACGTGGAGA |
| | GCAACCCCGGCCCCATGGAGGGCATAAGCAATTTCAAGACCCCCAGCAA |
| | GCTGAGCGAAAAGAAAAAGGGCAAGCCCATTCCCAACCCCCTTCTGGGCC |
| | TTGACAGCACC |
| | (TandemPep.TOPK (ORF)) |
| 376 | MEGISNFKTPSKLSEKKK |
| | (Isolated TOPK inhibitory peptide) |
| 377 | MMEGISNFKTPSKLSEKKKGSGVKQTLNFDLLKLAGDVESNPGPMEGISNFK |
| | TPSKLSEKKKGSGVKQTLNFDLLKLAGDVESNPGPMEGISNFKTPSKLSEKKK |
| | GKPIPNPLLGLDST (TOPK 3-peptide tandem with F2A linker) |
| 378 | MMEGISNFKTPSKLSEKKKGSGATNFSLLKQAGDVEENPGPMEGISNFKTPSK |
| | LSEKKKGSGATNFSLLKQAGDVEENPGPMEGISNFKTPSKLSEKKKGKPIPNP |
| | LLGLDST (TOPK 3-peptide tandem with P2A linker) |
| 379 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGATGA |
| | GCAGACGTAAGCAGGCTAAGCCCCAGCATATCGGCAGCGGCGTGAAGCA |
| | GACCCTGAACTTCGACCTGCTCAAGCTGGCCGGCGATGTCGAGTCAAACC |
| | CCGGCCCCATGAGCAGAAGAAAGCAGGCCAAGCCCCAGCACATCGGTAG |
| | CGGAGTGAAACAGACCCTGAACTTCGACTTACTGAAGCTCGCTGGCGACG |
| | TGGAGAGCAACCCCGGCCCCATGAGCAGAAGAAAGCAGGCCAAGCCCCA |
| | GCACATCGGAAAGCCCATCCCCAACCCCCTGCTGGGCCTGGACAGCACCT |
| | GATAATAGGCTGGAGCCTCGGTGGCCATGCTTCTTGCCCCTTGGGCCTCCC |
| | CCCAGCCCCTCCTCCCCTTCCTGCACCCGTACCCCCGTGGTCTTTGAATAA |
| | AGTCTGAGTGGGCGGC (TandemPep.Sall4, (5′ UTR, ORF, 3′ UTR)) |
| 380 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGATGA |
| | GCCGCAGAAAGCAGGCCAAGCCTCAGCATATCGGATCCGGCGTGAAGCA |
| | GACCCTGAACTTCGACCTTCTGAAGCTGGCCGGCGATGTGGAATCCAACC |
| | CGGGGCCCATGTCCCGGAGGAAACAAGCGAAGCCACAGCACATCGGATC |
| | GGGAGTGAAGCAAACTCTCAACTTCGACTTGCTGAAACTCGCCGGGGATG |
| | TCGAGTCAAATCCCGGCCCTATGAGCCGCCGGAAGCAGGCTAAGCCGCAG |
| | CACATTGGAAAGCCTATCCCCAACCCGCTGCTGGGTCTGGACAGCACCTG |
| | ATAATAGGCTGGAGCCTCGGTGGCCATGCTTCTTGCCCCTTGGGCCTCCCC |
| | CCAGCCCCTCCTCCCCTTCCTGCACCCGTACCCCCGTGGTCTTTGAATAAA |
| | GTCTGAGTGGGCGGC |
| | (TandemPep.Sall4 codon optimized,(5′ UTR, ORF, 3′ UTR)) |
| 381 | ATGATGAGCAGACGTAAGCAGGCTAAGCCCCAGCATATCGGCAGCGGCG |
| | TGAAGCAGACCCTGAACTTCGACCTGCTCAAGCTGGCCGGCGATGTCGAG |
| | TCAAACCCCGGCCCCATGAGCAGAAGAAAGCAGGCCAAGCCCCAGCACA |
| | TCGGTAGCGGAGTGAAACAGACCCTGAACTTCGACTTACTGAAGCTCGCT |
| | GGCGACGTGGAGAGCAACCCCGGCCCCATGAGCAGAAGAAAGCAGGCCA |
| | AGCCCCAGCACATCGGAAAGCCCATCCCCAACCCCCTGCTGGGCCTGGAC |
| | AGCACC (TandemPep.Sall4, (ORF)) |
| 382 | ATGATGAGCCGCAGAAAGCAGGCCAAGCCTCAGCATATCGGATCCGGCG |
| | TGAAGCAGACCCTGAACTTCGACCTTCTGAAGCTGGCCGGCGATGTGGAA |
| | TCCAACCCGGGGCCCATGTCCCGGAGGAAACAAGCGAAGCCACAGCACA |
| | TCGGATCGGGAGTGAAGCAAACTCTCAACTTCGACTTGCTGAAACTCGCC |
| | GGGGATGTCGAGTCAAATCCCGGCCCTATGAGCCGCCGGAAGCAGGCTA |
| | AGCCGCAGCACATTGGAAAGCCTATCCCCAACCCGCTGCTGGGTCTGGAC |
| | AGCACC (TandemPep.Sall4 codon optimized,(ORF,)) |
| 383 | MSRRKQAKPQHI (isolated SALL4-inhibitory peptide) |
| 384 | MMSRRKQAKPQHIGSGVKQTLNFDLLKLAGDVESNPGPMSRRKQAKPQHIG |
| | SGVKQTLNFDLLKLAGDVESNPGPMSRRKQAKPQHIGKPIPNPLLGLDST |
| | (SALL4-inhibitory 3-peptide tandem with F2A linker) |
| 385 | MMSRRKQAKPQHIGSGATNFSLLKQAGDVEENPGPMSRRKQAKPQHIGSGA |
| | TNFSLLKQAGDVEENPGPMSRRKQAKPQHIGKPIPNPLLGLDST |
| | (SALL4-inhibitory 3-peptide tandem with P2A linker) |
| 386 | HYPWFKARLYPL (Ras inhibitory peptide) |
| 387 | MHYPWFKARLYPLGSGVKQTLNFDLLKLAGDVESNPGPHYPWFKARLYPLG |
| | SGVKQTLNFDLLKLAGDVESNPGPHYPWFKARLYPLGKPIPNPLLGLDST (Ras |
| | inhibitory peptide tandem-(3x) (GSG)F2A linker-V5 Tag (optional)) |
| 388 | MHYPWFKARLYPLGSGATNFSLLKQAGDVEENPGPHYPWFKARLYPLGSGA |
| | TNFSLIKQAGDVEENPG PHYPWFKARLYPLGKPIPNPLLGLDST |
| | (Ras inhibitory peptide tandem-(3x) (GSG) P2A linker-V5 |
| | Tag (optional)) |
| 389 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGCACT |
| | ACCCCTGGTTCAAGGCCAGACTGTACCCCCTGGGCAGCGGCGTGAAGCAG |
| | ACCCTGAACTTCGACCTCCTGAAGCTGGCCGGCGATGTGGAGAGCAATCC |
| | CGGCCCCCACTACCCCTGGTTCAAGGCCAGACTGTACCCCCTGGGCAGCG |
| | GAGTGAAGCAAACCCTAAACTTTGACCTGCTGAAGCTGGCCGGCGACGTG |
| | GAAAGCAACCCCGGACCCCACTACCCCTGGTTTAAGGCCCGCCTGTACCC |
| | CCTGGGCAAGCCCATCCCCAATCCCCTGCTGGGCCTGGACAGCACCTGAT |
| | AATAGGCTGGAGCCTCGGTGGCCATGCTTCTTGCCCCTTGGGCCTCCCCCC |
| | AGCCCCTCCTCCCCTTCCTGCACCCGTACCCCCGTGGTCTTTGAATAAAGT |
| | CTGAGTGGGCGGC (Ras inhibitory peptide tandem-(3x)) |
| 390 | ATGCACTACCCCTGGTTCAAGGCCAGACTGTACCCCCTGGGCAGCGGCGT |
| | GAAGCAGACCCTGAACTTCGACCTCCTGAAGCTGGCCGGCGATGTGGAGA |
| | GCAATCCCGGCCCCCACTACCCCTGGTTCAAGGCCAGACTGTACCCCCTG |
| | GGCAGCGGAGTGAAGCAAACCCTAAACTTTGACCTGCTGAAGCTGGCCGG |
| | CGACGTGGAAAGCAACCCCGGACCCCACTACCCCTGGTTTAAGGCCCGCC |
| | TGTACCCCCTGGGCAAGCCCATCCCCAATCCCCTGCTGGGCCTGGACAGC |
| | ACC (Ras inhibitory peptide tandem-(3x) ORF) |
| 391 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGCATT |
| | ACCCGTGGTTTAAGGCCCGCCTGTATCCACTGGGATCCGGGGTCAAGCAG |
| | ACCCTTAACTTCGACCTCCTGAAGCTGGCCGGAGATGTGGAAAGCAACCC |
| | GGGTCCCCACTACCCCTGGTTCAAAGCGCGGCTGTACCCCCTGGGATCGG |
| | GCGTGAAGCAGACTCTCAACTTCGACTTGCTTAAGCTGGCTGGGGACGTG |
| | GAGTCCAATCCTGGCCCACACTACCCTTGGTTCAAGGCCCGGCTCTACCCT |
| | CTCGGAAAGCCGATCCCGAACCCCCTGCTGGGCCTGGATAGCACCTGATA |
| | ATAGGCTGGAGCCTCGGTGGCCATGCTTCTTGCCCCTTGGGCCTCCCCCCA |
| | GCCCCTCCTCCCCTTCCTGCACCCGTACCCCCGTGGTCTTTGAATAAAGTC |
| | TGAGTGGGCGGC |
| | (Ras inhibitory peptide tandem-(3x) codon optimized) |
| 392 | ATGCATTACCCGTGGTTTAAGGCCCGCCTGTATCCACTGGGATCCGGGGT |
| | CAAGCAGACCCTTAACTTCGACCTCCTGAAGCTGGCCGGAGATGTGGAAA |
| | GCAACCCGGGTCCCCACTACCCCTGGTTCAAAGCGCGGCTGTACCCCCTG |
| | GGATCGGGCGTGAAGCAGACTCTCAACTTCGACTTGCTTAAGCTGGCTGG |
| | GGACGTGGAGTCCAATCCTGGCCCACACTACCCTTGGTTCAAGGCCCGGC |
| | TCTACCCTCTCGGAAAGCCGATCCCGAACCCCCTGCTGGGCCTGGATAGC |
| | ACC |
| | (Ras inhibitory peptide tandem-(3x) codon optimized ORF) |
| 393 | METFSDLWKLLPEGSGVKQTLNFDLLKLAGDVESNPGPETFSDLWKLLPEGS |
| | GVKQTLNFDLLKLAGDVESNPGPETFSDLWKLLPEGKPIPNPLLGLDST |
| | (TandemPep.P53 ORF) |
| 394 | MLTFEHSWAQLTSGSGVKQTLNFDLLKLAGDVESNPGPLTFEHSWAQLTSGS |
| | GVKQTLNFDLLKLAGDVESNPGPLTFEHSWAQLTSGKPIPNPLLGLDST |
| | (TandemPep.p53.6S ORF) |
| 395 | METFEHWWAQLTSGSGVKQTLNFDLLKLAGDVESNPGPETFEHWWAQLTS |
| | GSGVKQTLNFDLLKLAGDVESNPGPETFEHWWAQLTSGKPIPNPLLGLDST |
| | (TandemPep.p53.P1E6W ORF) |
| 396 | MLTFEHWWAQLTSGSGVKQTLNFDLLKLAGDVESNPGPLTFEHWWAQLTS |
| | GSGVKQTLNFDLLKLAGDVESNPGPLTFEHWWAQLTSGKPIPNPLLGLDST |
| | (TandemPep.p53.P6W ORF) |
| 397 | METFEHWWSQLLSGSGVKQTLNFDLLKLAGDVESNPGPETFEHWWSQLLSG |
| | SGVKQTLNFDLLKLAGDVESNPGPETFEHWWSQLLSGKPIPNPLLGLDST |
| | (TandemPep.p53.pDIQ ORF) |
| 398 | MTSFAEYWNLLSPGSGVKQTLNFDLLKLAGDVESNPGPTSFAEYWNLLSPGS |
| | GVKQTLNFDLLKLAGDVESNPGPTSFAEYWNLLSPGKPIPNPLLGLDST |
| | (TandemPep.p53.pMI ORF) |
| 399 | TSFAEYWNLLSP (pMI) |
| 400 | ETFSDLWKLLPE (p53) |
| 401 | ETFEHWWSQLLS (pDIQ) |
| 402 | ETFEHWWAQLTS (p1E6W) |
| 403 | LTFEHSWAQLTS (p536S) |
| 404 | LTFEHWWAQLTS (P6W) |
| 405 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGGAG |
| | ACCTTTAGCGACCTGTGGAAGTTGCTGCCCGAGGGCAGCGGCGTGAAGCA |
| | GACCCTGAACTTCGATCTGCTGAAGTTAGCCGGGGACGTGGAGAGCAACC |
| | CCGGCCCCGAGACATTCTCAGACCTCTGGAAGCTGCTGCCCGAGGGCTCT |
| | GGCGTGAAGCAGACCCTGAACTTCGATTTACTGAAGCTGGCCGGCGACGT |
| | CGAGAGCAACCCCGGCCCCGAAACCTTCAGCGACCTGTGGAAGCTGCTGC |
| | CTGAGGGCAAGCCCATCCCCAACCCCCTGCTGGGCCTGGACAGCACCTGA |
| | TAATAGGCTGGAGCCTCGGTGGCCATGCTTCTTGCCCCTTGGGCCTCCCCC |
| | CAGCCCCTCCTCCCCTTCCTGCACCCGTACCCCCGTGGTCTTTGAATAAAG |
| | TCTGAGTGGGCGGC (TandemPep.P53 (5′UTR, ORF, 3′UTR) |
| 406 | ATGGAGACCTTTAGCGACCTGTGGAAGTTGCTGCCCGAGGGCAGCGGCGT |
| | GAAGCAGACCCTGAACTTCGATCTGCTGAAGTTAGCCGGGGACGTGGAGA |
| | GCAACCCCGGCCCCGAGACATTCTCAGACCTCTGGAAGCTGCTGCCCGAG |
| | GGCTCTGGCGTGAAGCAGACCCTGAACTTCGATTTACTGAAGCTGGCCGG |
| | CGACGTCGAGAGCAACCCCGGCCCCGAAACCTTCAGCGACCTGTGGAAGC |
| | TGCTGCCTGAGGGCAAGCCCATCCCCAACCCCCTGCTGGGCCTGGACAGC |
| | ACC (TandemPep.P53 ORF) |
| 407 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGCTGA |
| | CCTTCGAGCACAGCTGGGCTCAGCTGACCAGCGGCAGCGGCGTTAAGCAA |
| | ACCCTGAACTTCGATTTGCTGAAGCTCGCCGGCGACGTGGAAAGCAACCC |
| | TGGCCCCCTGACCTTCGAGCATTCTTGGGCTCAGCTGACCAGCGGGTCTG |
| | GCGTGAAGCAGACCCTGAACTTCGACCTGTTAAAGCTGGCCGGAGATGTG |
| | GAGAGCAATCCCGGCCCCCTGACCTTCGAGCACAGTTGGGCCCAGCTGAC |
| | CAGCGGCAAGCCCATCCCCAACCCCCTGTTGGGCCTGGACAGCACCTGAT |
| | AATAGGCTGGAGCCTCGGTGGCCATGCTTCTTGCCCCTTGGGCCTCCCCCC |
| | AGCCCCTCCTCCCCTTCCTGCACCCGTACCCCCGTGGTCTTTGAATAAAGT |
| | CTGAGTGGGCGGC (TandemPep.P53.6S (5′UTR, ORF, 3′UTR) |
| 408 | ATGCTGACCTTCGAGCACAGCTGGGCTCAGCTGACCAGCGGCAGCGGCGT |
| | TAAGCAAACCCTGAACTTCGATTTGCTGAAGCTCGCCGGCGACGTGGAAA |
| | GCAACCCTGGCCCCCTGACCTTCGAGCATTCTTGGGCTCAGCTGACCAGC |
| | GGGTCTGGCGTGAAGCAGACCCTGAACTTCGACCTGTTAAAGCTGGCCGG |
| | AGATGTGGAGAGCAATCCCGGCCCCCTGACCTTCGAGCACAGTTGGGCCC |
| | AGCTGACCAGCGGCAAGCCCATCCCCAACCCCCTGTTGGGCCTGGACAGC |
| | ACC (TandemPep.P53.6S ORF) |
| 409 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGGAG |
| | ACCTTCGAGCACTGGTGGGCGCAACTGACCAGCGGCAGCGGCGTGAAGC |
| | AGACCCTGAACTTCGATCTGCTGAAGTTAGCCGGCGACGTGGAGTCAAAC |
| | CCCGGCCCCGAGACCTTCGAGCACTGGTGGGCCCAGCTGACCTCTGGCAG |
| | CGGCGTCAAGCAAACCCTGAACTTCGACCTTCTGAAGCTGGCCGGCGATG |
| | TGGAGAGCAACCCCGGCCCCGAGACCTTCGAGCACTGGTGGGCCCAGCTC |
| | ACCTCGGGCAAGCCCATCCCCAACCCCCTGCTGGGTCTGGACAGCACCTG |
| | ATAATAGGCTGGAGCCTCGGTGGCCATGCTTCTTGCCCCTTGGGCCTCCCC |
| | CCAGCCCCTCCTCCCCTTCCTGCACCCGTACCCCCGTGGTCTTTGAATAAA |
| | GTCTGAGTGGGCGGC (Tandem.Pep.P53.P1.E6W (5′UTR, ORF, 3′UTR) |
| 410 | ATGGAGACCTTCGAGCACTGGTGGGCGCAACTGACCAGCGGCAGCGGCG |
| | TGAAGCAGACCCTGAACTTCGATCTGCTGAAGTTAGCCGGCGACGTGGAG |
| | TCAAACCCCGGCCCCGAGACCTTCGAGCACTGGTGGGCCCAGCTGACCTC |
| | TGGCAGCGGCGTCAAGCAAACCCTGAACTTCGACCTTCTGAAGCTGGCCG |
| | GCGATGTGGAGAGCAACCCCGGCCCCGAGACCTTCGAGCACTGGTGGGCC |
| | CAGCTCACCTCGGGCAAGCCCATCCCCAACCCCCTGCTGGGTCTGGACAG |
| | CACC (Tandem.Pep.P53.P1.E6W ORF) |
| 411 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGGAA |
| | ACCTTCGAGCATTGGTGGGCACAGCTGACCAGCGGATCAGGAGTGAAAC |
| | AGACTCTCAACTTCGATCTGCTGAAGCTTGCCGGGGACGTGGAGTCCAAC |
| | CCTGGCCCTGAAACTTTCGAACATTGGTGGGCTCAGCTCACCTCCGGCTCG |
| | GGAGTCAAGCAGACTCTGAACTTCGACCTCCTGAAGTTGGCCGGCGATGT |
| | GGAGAGCAACCCCGGTCCCGAAACCTTTGAGCACTGGTGGGCGCAACTGA |
| | CCTCCGGAAAGCCGATCCCAAATCCGCTGCTGGGGCTGGACAGCACCTGA |
| | TAATAGGCTGGAGCCTCGGTGGCCATGCTTCTTGCCCCTTGGGCCTCCCCC |
| | CAGCCCCTCCTCCCCTTCCTGCACCCGTACCCCCGTGGTCTTTGAATAAAG |
| | TCTGAGTGGGCGGC |
| | (TandemPep.p53.P1E6W_DNA2.0 (5′UTR, ORF, 3′UTR) |
| 412 | ATGGAAACCTTCGAGCATTGGTGGGCACAGCTGACCAGCGGATCAGGAGT |
| | GAAACAGACTCTCAACTTCGATCTGCTGAAGCTTGCCGGGGACGTGGAGT |
| | CCAACCCTGGCCCTGAAACTTTCGAACATTGGTGGGCTCAGCTCACCTCC |
| | GGCTCGGGAGTCAAGCAGACTCTGAACTTCGACCTCCTGAAGTTGGCCGG |
| | CGATGTGGAGAGCAACCCCGGTCCCGAAACCTTTGAGCACTGGTGGGCGC |
| | AACTGACCTCCGGAAAGCCGATCCCAAATCCGCTGCTGGGGCTGGACAGC |
| | ACC (TandemPep.p53.P1E6W_DNA2.0 ORF) |
| 413 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGCTGA |
| | CCTTTGAGCACTGGTGGGCCCAGCTGACCAGCGGCAGCGGCGTGAAACAG |
| | ACCCTGAACTTCGACCTGCTGAAACTGGCCGGCGACGTGGAGTCTAACCC |
| | CGGGCCCCTGACCTTCGAGCACTGGTGGGCCCAGCTGACCAGCGGAAGCG |
| | GAGTGAAGCAGACGCTAAACTTCGACCTCCTGAAGCTGGCCGGCGATGTG |
| | GAGAGCAACCCCGGACCCCTGACCTTCGAGCACTGGTGGGCCCAGCTGAC |
| | TAGCGGCAAGCCCATCCCCAATCCCCTGCTGGGCCTGGACAGCACCTGAT |
| | AATAGGCTGGAGCCTCGGTGGCCATGCTTCTTGCCCCTTGGGCCTCCCCCC |
| | AGCCCCTCCTCCCCTTCCTGCACCCGTACCCCCGTGGTCTTTGAATAAAGT |
| | CTGAGTGGGCGGC |
| | (TandemPep.P53.P6W (5′UTR, ORF, 3′UTR) |
| 414 | ATGCTGACCTTTGAGCACTGGTGGGCCCAGCTGACCAGCGGCAGCGGCGT |
| | GAAACAGACCCTGAACTTCGACCTGCTGAAACTGGCCGGCGACGTGGAGT |
| | CTAACCCCGGGCCCCTGACCTTCGAGCACTGGTGGGCCCAGCTGACCAGC |
| | GGAAGCGGAGTGAAGCAGACGCTAAACTTCGACCTCCTGAAGCTGGCCG |
| | GCGATGTGGAGAGCAACCCCGGACCCCTGACCTTCGAGCACTGGTGGGCC |
| | CAGCTGACTAGCGGCAAGCCCATCCCCAATCCCCTGCTGGGCCTGGACAG |
| | CACC (TandemPep.P53.P6W ORF) |
| 415 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGCTCA |
| | CCTTCGAACACTGGTGGGCGCAGCTGACCTCAGGCTCCGGAGTGAAGCAG |
| | ACCCTGAACTTCGATCTGCTCAAGCTGGCTGGCGATGTGGAAAGCAACCC |
| | GGGGCCCCTCACTTTCGAGCATTGGTGGGCCCAGTTGACTTCGGGCTCCG |
| | GTGTCAAGCAAACCCTCAATTTCGACCTTCTGAAGCTGGCCGGGGACGTG |
| | GAGAGCAACCCAGGACCTCTGACCTTTGAACATTGGTGGGCACAGCTGAC |
| | TTCCGGAAAACCCATCCCGAACCCTCTGCTGGGACTGGACAGCACCTGAT |
| | AATAGGCTGGAGCCTCGGTGGCCATGCTTCTTGCCCCTTGGGCCTCCCCCC |
| | AGCCCCTCCTCCCCTTCCTGCACCCGTACCCCCGTGGTCTTTGAATAAAGT |
| | CTGAGTGGGCGGC |
| | (TandemPep.p53.P6W_DNA2.0 (5′UTR, ORF, 3′UTR) |
| 416 | ATGCTCACCTTCGAACACTGGTGGGCGCAGCTGACCTCAGGCTCCGGAGT |
| | GAAGCAGACCCTGAACTTCGATCTGCTCAAGCTGGCTGGCGATGTGGAAA |
| | GCAACCCGGGGCCCCTCACTTTCGAGCATTGGTGGGCCCAGTTGACTTCG |
| | GGCTCCGGTGTCAAGCAAACCCTCAATTTCGACCTTCTGAAGCTGGCCGG |
| | GGACGTGGAGAGCAACCCAGGACCTCTGACCTTTGAACATTGGTGGGCAC |
| | AGCTGACTTCCGGAAAACCCATCCCGAACCCTCTGCTGGGACTGGACAGC |
| | ACC (TandemPep.p53.P6W_DNA2.0 ORF) |
| 417 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGGAG |
| | ACCTTCGAGCACTGGTGGAGCCAGCTGCTTAGCGGAAGCGGAGTGAAAC |
| | AAACCTTAAACTTCGACCTGCTGAAGCTGGCCGGCGACGTGGAAAGCAAT |
| | CCCGGCCCCGAGACCTTCGAGCACTGGTGGAGCCAGCTGTTGAGCGGTAG |
| | CGGCGTGAAGCAGACCCTGAACTTCGACCTGCTCAAGCTGGCCGGCGACG |
| | TGGAGAGCAACCCCGGCCCAGAGACCTTCGAGCACTGGTGGAGCCAGCT |
| | GCTGAGCGGCAAGCCCATCCCCAATCCCCTGCTGGGCCTGGACAGCACCT |
| | GATAATAGGCTGGAGCCTCGGTGGCCATGCTTCTTGCCCCTTGGGCCTCCC |
| | CCCAGCCCCTCCTCCCCTTCCTGCACCCGTACCCCCGTGGTCTTTGAATAA |
| | AGTCTGAGTGGGCGGC |
| | (TandemPep.p53.pDIQ (5′UTR, ORF, 3′UTR) |
| 418 | ATGGAGACCTTCGAGCACTGGTGGAGCCAGCTGCTTAGCGGAAGCGGAGT |
| | GAAACAAACCTTAAACTTCGACCTGCTGAAGCTGGCCGGCGACGTGGAAA |
| | GCAATCCCGGCCCCGAGACCTTCGAGCACTGGTGGAGCCAGCTGTTGAGC |
| | GGTAGCGGCGTGAAGCAGACCCTGAACTTCGACCTGCTCAAGCTGGCCGG |
| | CGACGTGGAGAGCAACCCCGGCCCAGAGACCTTCGAGCACTGGTGGAGC |
| | CAGCTGCTGAGCGGCAAGCCCATCCCCAATCCCCTGCTGGGCCTGGACAG |
| | CACC (TandemPep.p53.pDIQ ORF) |
| 419 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGGAA |
| | ACCTTTGAACACTGGTGGAGCCAGCTCCTGTCGGGATCCGGCGTGAAGCA |
| | AACTCTGAATTTCGACCTGTTGAAACTTGCGGGGGACGTCGAGAGCAACC |
| | CGGGTCCTGAAACTTTCGAGCATTGGTGGTCCCAGCTGCTGTCAGGCTCC |
| | GGAGTGAAGCAGACCCTCAACTTCGATCTGCTGAAGCTGGCCGGCGATGT |
| | GGAGTCCAACCCGGGACCCGAAACCTTCGAGCACTGGTGGTCGCAGCTGC |
| | TCTCCGGGAAGCCAATCCCCAACCCTCTGCTCGGACTGGACAGCACCTGA |
| | TAATAGGCTGGAGCCTCGGTGGCCATGCTTCTTGCCCCTTGGGCCTCCCCC |
| | CAGCCCCTCCTCCCCTTCCTGCACCCGTACCCCCGTGGTCTTTGAATAAAG |
| | TCTGAGTGGGCGGC |
| | (TandemPep.p53.pDIQ_DNA2.0 (5′UTR, ORF, 3′UTR) |
| 420 | ATGGAAACCTTTGAACACTGGTGGAGCCAGCTCCTGTCGGGATCCGGCGT |
| | GAAGCAAACTCTGAATTTCGACCTGTTGAAACTTGCGGGGGACGTCGAGA |
| | GCAACCCGGGTCCTGAAACTTTCGAGCATTGGTGGTCCCAGCTGCTGTCA |
| | GGCTCCGGAGTGAAGCAGACCCTCAACTTCGATCTGCTGAAGCTGGCCGG |
| | CGATGTGGAGTCCAACCCGGGACCCGAAACCTTCGAGCACTGGTGGTCGC |
| | AGCTGCTCTCCGGGAAGCCAATCCCCAACCCTCTGCTCGGACTGGACAGC |
| | ACC (TandemPep.p53.pDIQ_DNA2.0 ORF) |
| 421 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGACCT |
| | CCTTCGCCGAGTACTGGAACCTGCTGAGCCCTGGCAGCGGCGTTAAACAG |
| | ACCCTGAATTTCGACCTGCTGAAGCTGGCCGGCGATGTGGAGAGCAACCC |
| | CGGCCCCACCAGCTTCGCTGAATACTGGAACTTGCTGAGCCCCGGCTCAG |
| | GCGTGAAGCAGACCCTGAACTTCGACTTACTGAAACTGGCCGGCGACGTG |
| | GAAAGCAATCCCGGCCCCACCAGCTTCGCAGAGTACTGGAACCTGCTGAG |
| | CCCCGGCAAGCCCATCCCCAACCCCCTGCTCGGCCTGGACAGCACCTGAT |
| | AATAGGCTGGAGCCTCGGTGGCCATGCTTCTTGCCCCTTGGGCCTCCCCCC |
| | AGCCCCTCCTCCCCTTCCTGCACCCGTACCCCCGTGGTCTTTGAATAAAGT |
| | CTGAGTGGGCGGC (TandemPep.p53.pMI (5′UTR, ORF, 3′UTR) |
| 422 | ATGACCTCCTTCGCCGAGTACTGGAACCTGCTGAGCCCTGGCAGCGGCGT |
| | TAAACAGACCCTGAATTTCGACCTGCTGAAGCTGGCCGGCGATGTGGAGA |
| | GCAACCCCGGCCCCACCAGCTTCGCTGAATACTGGAACTTGCTGAGCCCC |
| | GGCTCAGGCGTGAAGCAGACCCTGAACTTCGACTTACTGAAACTGGCCGG |
| | CGACGTGGAAAGCAATCCCGGCCCCACCAGCTTCGCAGAGTACTGGAACC |
| | TGCTGAGCCCCGGCAAGCCCATCCCCAACCCCCTGCTCGGCCTGGACAGC |
| | ACC (TandemPep.p53.pMI ORF) |
| 423 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGACCA |
| | GCTTCGCCGAATACTGGAACCTCTTGTCCCCCGGATCGGGGGTCAAGCAG |
| | ACCCTGAACTTTGATCTCCTGAAGCTGGCCGGGGATGTGGAAAGCAACCC |
| | CGGACCTACTTCCTTCGCCGAGTACTGGAATCTGCTGTCGCCGGGATCCG |
| | GCGTGAAGCAAACTCTGAACTTCGACCTCCTTAAACTTGCGGGCGACGTG |
| | GAGTCCAACCCGGGCCCTACCTCATTCGCTGAATATTGGAACCTCCTGTCC |
| | CCGGGAAAGCCCATCCCTAACCCACTGCTGGGTCTGGACAGCACCTGATA |
| | ATAGGCTGGAGCCTCGGTGGCCATGCTTCTTGCCCCTTGGGCCTCCCCCCA |
| | GCCCCTCCTCCCCTTCCTGCACCCGTACCCCCGTGGTCTTTGAATAAAGTC |
| | TGAGTGGGCGGC (TandemPep.p53.pMI_DNA2.0 (5′UTR, ORF, 3′UTR) |
| 424 | ATGACCAGCTTCGCCGAATACTGGAACCTCTTGTCCCCCGGATCGGGGGT |
| | CAAGCAGACCCTGAACTTTGATCTCCTGAAGCTGGCCGGGGATGTGGAAA |
| | GCAACCCCGGACCTACTTCCTTCGCCGAGTACTGGAATCTGCTGTCGCCG |
| | GGATCCGGCGTGAAGCAAACTCTGAACTTCGACCTCCTTAAACTTGCGGG |
| | CGACGTGGAGTCCAACCCGGGCCCTACCTCATTCGCTGAATATTGGAACC |
| | TCCTGTCCCCGGGAAAGCCCATCCCTAACCCACTGCTGGGTCTGGACAGC |
| | ACC ((TandemPep.p53.pMI_DNA2.0 ORF) |
| 425 | MTPDYFLGSGVKQTLNFDLLKLAGDVESNPGPTPDYFLGSGVKQTLNFDLLK |
| | LAGDVESNPGPTPDYFLGKPIPNPLLGLDST |
| | (TandemPep.PP2aB56alpha, ORF) |
| 426 | MVKKKKIKREIKIFRGRSRFRGRSRGSGVKQTLNFDLLLAGDVESNPGPVKK |
| | KKIKREIKIFRGRSRFRGRSRGSGVKQTLNFDLLKLAGDVESNPGPVKKKKIK |
| | REIKIFRGRSRFRGRSRGKPIPNPLLGLDST |
| | (TandemPep.PP2aDP7, ORF) |
| 427 | RQKRLIRQKRLIRQKRLIGSGVKQTLNFDLLKLAGDVESNPGPRQKRLIRQKR |
| | LIRQKRLIGSGVKQTLNFDLLKLAGDVESNPGPRQKRLIRQKRLIRQKRLIGKP |
| | IPNPLLGLDST |
| | (TandemPep.PP2aDPT2) |
| 428 | MVKKKKIKREIKIPRRPGPTRKHYQPYAGSGVKQTLNFDLLKLAGDVESNPG |
| | PVKKKKIKREIKIPRRPGPTRKHYQPYAGSGVKQTLNFDLLKLAGDVESNPGP |
| | VKKKKIKREIKIPRRPGPTRKHYQPYAGKPIPNPLLGLDST |
| | (TandemPep.PP2aDPT5) |
| 429 | TPDYFL (PP2aB56alpha ) |
| 430 | VKKKKIKREIKIFRGRSRFRGRSR (PP2aDP7) |
| 431 | RQKRLIRQKRLIRQKRLI (PP2aDPT2) |
| 432 | VKKKKIKREIKIPRRPGPTRKHYQPYA (PP2aDPT5) |
| 433 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGACCC |
| | CCGACTACTTCCTGGGCAGCGGCGTTAAGCAGACCCTGAACTTTGACCTA |
| | CTGAAGCTGGCCGGCGATGTGGAGTCTAACCCCGGCCCCACCCCCGACTA |
| | CTTCCTGGGCTCCGGCGTGAAGCAGACCCTGAATTTCGACCTGCTAAAGC |
| | TGGCCGGCGACGTGGAGAGCAACCCCGGCCCCACCCCCGACTACTTTCTG |
| | GGCAAGCCCATCCCCAACCCCCTGCTGGGCCTGGACAGCACCTGATAATA |
| | GGCTGGAGCCTCGGTGGCCATGCTTCTTGCCCCTTGGGCCTCCCCCCAGCC |
| | CCTCCTCCCCTTCCTGCACCCGTACCCCCGTGGTCTTTGAATAAAGTCTGA |
| | GTGGGCGGC |
| | (TandemPep.PP2aB56alpha (5′ UTR, ORF, 3′ UTR) |
| 434 | ATGACCCCCGACTACTTCCTGGGCAGCGGCGTTAAGCAGACCCTGAACTT |
| | TGACCTACTGAAGCTGGCCGGCGATGTGGAGTCTAACCCCGGCCCCACCC |
| | CCGACTACTTCCTGGGCTCCGGCGTGAAGCAGACCCTGAATTTCGACCTG |
| | CTAAAGCTGGCCGGCGACGTGGAGAGCAACCCCGGCCCCACCCCCGACTA |
| | CTTTCTGGGCAAGCCCATCCCCAACCCCCTGCTGGGCCTGGACAGCACC |
| | (TandemPep.PP2aB56a1ph ORF) |
| 435 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGGTGA |
| | AGAAGAAGAAGATCAAGCGGGAGATCAAGATCTTCAGAGGAAGAAGCCG |
| | GTTCAGAGGGAGAAGCAGAGGCAGCGGCGTGAAGCAGACGCTGAATTTT |
| | GACCTGTTGAAGCTGGCAGGAGACGTGGAGTCCAACCCCGGTCCCGTGAA |
| | GAAGAAGAAAATCAAAAGAGAAATCAAGATCTTCAGAGGCAGAAGCAGA |
| | TTCAGAGGCAGAAGCAGAGGCAGCGGCGTGAAACAAACCCTAAACTTCG |
| | ACCTGCTGAAGCTCGCCGGCGACGTGGAGAGCAACCCAGGCCCCGTGAA |
| | GAAGAAGAAGATTAAGAGAGAGATCAAGATCTTCAGAGGCCGCAGCAGA |
| | TTCAGAGGCAGATCTCGTGGCAAGCCCATCCCAAACCCCCTGCTGGGGCT |
| | CGACAGCACCTGATAATAGGCTGGAGCCTCGGTGGCCATGCTTCTTGCCC |
| | CTTGGGCCTCCCCCCAGCCCCTCCTCCCCTTCCTGCACCCGTACCCCCGTG |
| | GTCTTTGAATAAAGTCTGAGTGGGCGGC |
| | (TandemPep.PP2aDP7 (5′ UTR, ORF, 3′ UTR)) |
| 436 | ATGGTGAAGAAGAAGAAGATCAAGCGGGAGATCAAGATCTTCAGAGGAA |
| | GAAGCCGGTTCAGAGGGAGAAGCAGAGGCAGCGGCGTGAAGCAGACGCT |
| | GAATTTTGACCTGTTGAAGCTGGCAGGAGACGTGGAGTCCAACCCCGGTC |
| | CCGTGAAGAAGAAGAAAATCAAAAGAGAAATCAAGATCTTCAGAGGCAG |
| | AAGCAGATTCAGAGGCAGAAGCAGAGGCAGCGGCGTGAAACAAACCCTA |
| | AACTTCGACCTGCTGAAGCTCGCCGGCGACGTGGAGAGCAACCCAGGCCC |
| | CGTGAAGAAGAAGAAGATTAAGAGAGAGATCAAGATCTTCAGAGGCCGC |
| | AGCAGATTCAGAGGCAGATCTCGTGGCAAGCCCATCCCAAACCCCCTGCT |
| | GGGGCTCGACAGCACC |
| | (TandemPep.PP2aDP7ORF) |
| 437 | TCAAGCTTTTGGACCCTCGTACAGAAGCTAATACGACTCACTATAGGG |
| | AAATAAGAGAGAAAAGAAGAGTAAGAAGAAATATAAGAGCCACCATGA |
| | GACAGAAAAGACTGATTAGACAGAAGAGACTGATCAGACAGAAGAGACT |
| | GATCGGGAGCGGCGTTAAGCAGACCTTAAACTTCGATCTGCTGAAACTGG |
| | CCGGCGACGTGGAGAGTAACCCCGGGCCCAGACAGAAAAGACTCATCAG |
| | GCAGAAGAGACTGATCAGACAGAAGAGACTGATCGGCTCCGGCGTGAAA |
| | CAGACCCTGAATTTCGACCTGCTGAAGCTGGCCGGAGATGTCGAGAGCAA |
| | CCCCGGCCCCAGACAGAAGAGACTGATAAGACAAAAGAGATTGATCAGA |
| | CAGAAGAGACTGATCGGCAAGCCCATCCCCAACCCCCTGCTGGGCCTGGA |
| | CAGCACCTGATAATAGGCTGGAGCCTCGGTGGCCATGCTTCTTGCCCCTTG |
| | GGCCTCCCCCCAGCCCCTCCTCCCCTTCCTGCACCCGTACCCCCGTGGTCT |
| | TTGAATAAAGTCTGAGTGGGCGGC |
| | (TandemPep.PP2aDPT2 (5′ UTR, ORF, 3′ UTR)) |
| 438 | ATGAGACAGAAAAGACTGATTAGACAGAAGAGACTGATCAGACAGAAGA |
| | GACTGATCGGGAGCGGCGTTAAGCAGACCTTAAACTTCGATCTGCTGAAA |
| | CTGGCCGGCGACGTGGAGAGTAACCCCGGGCCCAGACAGAAAAGACTCA |
| | TCAGGCAGAAGAGACTGATCAGACAGAAGAGACTGATCGGCTCCGGCGT |
| | GAAACAGACCCTGAATTTCGACCTGCTGAAGCTGGCCGGAGATGTCGAGA |
| | GCAACCCCGGCCCCAGACAGAAGAGACTGATAAGACAAAAGAGATTGAT |
| | CAGACAGAAGAGACTGATCGGCAAGCCCATCCCCAACCCCCTGCTGGGCC |
| | TGGACAGCACC |
| | (TandemPep.PP2aDPT2 ORF) |
| 439 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGGTCA |
| | AAAAGAAGAAGATCAAAAGAGAGATCAAGATCCCCAGAAGACCCGGGCC |
| | AACAAGGAAACACTACCAGCCCTACGCCGGCAGCGGGGTGAAACAGACC |
| | CTGAACTTCGATCTGCTGAAGTTGGCCGGGGACGTCGAGTCCAATCCCGG |
| | CCCCGTGAAGAAGAAAAAGATCAAGAGGGAGATCAAGATCCCCAGAAGA |
| | CCCGGTCCCACCCGGAAGCACTACCAGCCCTACGCGGGCTCCGGCGTGAA |
| | GCAGACCCTGAATTTCGACCTGCTGAAACTGGCCGGTGACGTGGAGAGCA |
| | ACCCTGGCCCCGTGAAGAAGAAGAAGATTAAGAGAGAGATCAAGATCCC |
| | CAGAAGACCCGGCCCCACCAGAAAGCACTACCAACCCTATGCCGGCAAG |
| | CCCATCCCAAACCCCCTGCTGGGACTGGACAGCACCTGATAATAGGCTGG |
| | AGCCTCGGTGGCCATGCTTCTTGCCCCTTGGGCCTCCCCCCAGCCCCTCCT |
| | CCCCTTCCTGCACCCGTACCCCCGTGGTCTTTGAATAAAGTCTGAGTGGGC |
| | GGC |
| | (TandemPep.PP2aDPT5 (5′ UTR, ORF, 3′ UTR)) |
| 440 | ATGGTCAAAAAGAAGAAGATCAAAAGAGAGATCAAGATCCCCAGAAGAC |
| | CCGGGCCAACAAGGAAACACTACCAGCCCTACGCCGGCAGCGGGGTGAA |
| | ACAGACCCTGAACTTCGATCTGCTGAAGTTGGCCGGGGACGTCGAGTCCA |
| | ATCCCGGCCCCGTGAAGAAGAAAAAGATCAAGAGGGAGATCAAGATCCC |
| | CAGAAGACCCGGTCCCACCCGGAAGCACTACCAGCCCTACGCGGGCTCCG |
| | GCGTGAAGCAGACCCTGAATTTCGACCTGCTGAAACTGGCCGGTGACGTG |
| | GAGAGCAACCCTGGCCCCGTGAAGAAGAAGAAGATTAAGAGAGAGATCA |
| | AGATCCCCAGAAGACCCGGCCCCACCAGAAAGCACTACCAACCCTATGCC |
| | GGCAAGCCCATCCCAAACCCCCTGCTGGGACTGGACAGCACC |
| | (TandemPep.PP2aDPT5 ORF) |
| 441 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGGTCA |
| | AGAAGAAGAAGATCAAGCGGGAGATTAAGATTCCTAGACGCCCCGGACC |
| | CACTAGAAAGCATTACCAGCCTTACGCGGGCTCCGGAGTGAAGCAAACCC |
| | TGAACTTCGACTTGCTGAAGCTGGCCGGGGACGTGGAGTCCAACCCCGGC |
| | CCGGTCAAGAAAAAGAAGATTAAGCGCGAAATCAAAATCCCCCGCCGGC |
| | CGGGGCCTACCCGGAAGCACTACCAGCCATACGCAGGAAGCGGCGTGAA |
| | GCAGACTCTGAATTTCGATCTCCTGAAGCTCGCCGGAGATGTGGAATCGA |
| | ACCCAGGTCCCGTGAAGAAGAAAAAGATCAAGAGGGAGATCAAGATCCC |
| | GAGGCGGCCGGGTCCCACCCGCAAGCACTATCAGCCGTACGCTGGAAAG |
| | CCTATCCCTAACCCGCTTCTGGGCCTGGACTCAACCTGATAATAGGCTGG |
| | AGCCTCGGTGGCCATGCTTCTTGCCCCTTGGGCCTCCCCCCAGCCCCTCCT |
| | CCCCTTCCTGCACCCGTACCCCCGTGGTCTTTGAATAAAGTCTGAGTGGGC |
| | GGC |
| | (TandemPep.PP2aDPT5_codon optimized (5′ UTR, ORF, 3′ UTR)) |
| 442 | ATGGTCAAGAAGAAGAAGATCAAGCGGGAGATTAAGATTCCTAGACGCC |
| | CCGGACCCACTAGAAAGCATTACCAGCCTTACGCGGGCTCCGGAGTGAAG |
| | CAAACCCTGAACTTCGACTTGCTGAAGCTGGCCGGGGACGTGGAGTCCAA |
| | CCCCGGCCCGGTCAAGAAAAAGAAGATTAAGCGCGAAATCAAAATCCCC |
| | CGCCGGCCGGGGCCTACCCGGAAGCACTACCAGCCATACGCAGGAAGCG |
| | GCGTGAAGCAGACTCTGAATTTCGATCTCCTGAAGCTCGCCGGAGATGTG |
| | GAATCGAACCCAGGTCCCGTGAAGAAGAAAAAGATCAAGAGGGAGATCA |
| | AGATCCCGAGGCGGCCGGGTCCCACCCGCAAGCACTATCAGCCGTACGCT |
| | GGAAAGCCTATCCCTAACCCGCTTCTGGGCCTGGACTCAACC |
| | (TandemPep.PP2aDPT5_codon optimized ORF) |
| 443 | MPLTAVFWLIYVLAKALVTVCGSGVKQTLNFDLLKLAGDVESNPGPPLTAVF |
| | WLIYVLAKALVTVCGSGVKQTLNFDLLKLAGDVESNPGPPLTAVFWLIYVLA |
| | KALVTVCGKPIPNPLLGLDST |
| | (TandemPep.STAT3.DBD ORF) |
| 444 | PLTAVFWLIYVLAKALVTVC (STAT3) |
| | TCAAGCTTTTGGACCCTCGTACAGAAGCTAATACGACTCACTATAGGGAA |
| | ATAAGAGAGAAAAGAAGAGTAAGAAGAAATATAAGAGCCACCATGCCCC |
| | TGACCGCCGTGTTCTGGCTGATCTACGTGTTGGCTAAGGCCCTGGTGACCG |
| | TGTGCGGAAGCGGAGTGAAACAGACCTTGAACTTTGACCTGCTGAAGCTG |
| | GCCGGAGACGTGGAAAGCAACCCCGGCCCCCCCCTGACCGCAGTGTTTTG |
| | GCTGATATACGTGCTGGCCAAGGCCCTGGTGACTGTGTGCGGCAGCGGCG |
| | TGAAGCAGACCCTGAACTTCGACCTGCTGAAGCTGGCCGGCGACGTCGAG |
| | AGCAACCCCGGCCCCCCCCTGACCGCCGTGTTCTGGTTGATATATGTGCTG |
| | GCCAAGGCCCTGGTGACAGTGTGCGGCAAGCCCATCCCAAACCCCCTGCT |
| | TGGCCTGGATAGCACCTGATAATAGGCTGGAGCCTCGGTGGCCATGCTTC |
| | TTGCCCCTTGGGCCTCCCCCCAGCCCCTCCTCCCCTTCCTGCACCCGTACC |
| | CCCGTGGTCTTTGAATAAAGTCTGAGTGGGCGGC (TandemPep.STAT3.DBD |
| | (5′ UTR, ORF, 3′ UTR)) |
| 445 | ATGCCCCTGACCGCCGTGTTCTGGCTGATCTACGTGTTGGCTAAGGCCCTG |
| | GTGACCGTGTGCGGAAGCGGAGTGAAACAGACCTTGAACTTTGACCTGCT |
| | GAAGCTGGCCGGAGACGTGGAAAGCAACCCCGGCCCCCCCCTGACCGCA |
| | GTGTTTTGGCTGATATACGTGCTGGCCAAGGCCCTGGTGACTGTGTGCGG |
| | CAGCGGCGTGAAGCAGACCCTGAACTTCGACCTGCTGAAGCTGGCCGGCG |
| | ACGTCGAGAGCAACCCCGGCCCCCCCCTGACCGCCGTGTTCTGGTTGATA |
| | TATGTGCTGGCCAAGGCCCTGGTGACAGTGTGCGGCAAGCCCATCCCAAA |
| | CCCCCTGCTTGGCCTGGATAGCACC (TandemPep.STAT3.DBD ORF) |
| 446 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGCCTC |
| | TGACGGCAGTGTTCTGGTTGATCTACGTGCTCGCCAAAGCCCTTGTGACCG |
| | TGTGTGGATCCGGCGTCAAGCAGACCCTCAATTTCGACTTGCTGAAGCTG |
| | GCCGGGGATGTGGAAAGCAACCCCGGACCCCCACTGACCGCCGTGTTTTG |
| | GCTCATCTACGTCCTGGCTAAGGCGCTCGTGACTGTGTGCGGTTCGGGCGT |
| | GAAGCAGACTCTGAACTTCGATCTGCTCAAGCTTGCCGGGGACGTGGAGT |
| | CCAACCCTGGACCTCCGCTGACCGCGGTGTTCTGGCTGATCTATGTGCTGG |
| | CCAAGGCCCTGGTCACCGTCTGCGGAAAGCCGATTCCCAACCCGCTGCTG |
| | GGCCTGGACAGCACCTGATAATAGGCTGGAGCCTCGGTGGCCATGCTTCT |
| | TGCCCCTTGGGCCTCCCCCCAGCCCCTCCTCCCCTTCCTGCACCCGTACCC |
| | CCGTGGTCTTTGAATAAAGTCTGAGTGGGCGGC |
| | (TandemPep.STAT3.DBD_codon optimized (5′ UTR, ORF, 3′ UTR) |
| 447 | ATGCCTCTGACGGCAGTGTTCTGGTTGATCTACGTGCTCGCCAAAGCCCTT |
| | GTGACCGTGTGTGGATCCGGCGTCAAGCAGACCCTCAATTTCGACTTGCT |
| | GAAGCTGGCCGGGGATGTGGAAAGCAACCCCGGACCCCCACTGACCGCC |
| | GTGTTTTGGCTCATCTACGTCCTGGCTAAGGCGCTCGTGACTGTGTGCGGT |
| | TCGGGCGTGAAGCAGACTCTGAACTTCGATCTGCTCAAGCTTGCCGGGGA |
| | CGTGGAGTCCAACCCTGGACCTCCGCTGACCGCGGTGTTCTGGCTGATCT |
| | ATGTGCTGGCCAAGGCCCTGGTCACCGTCTGCGGAAAGCCGATTCCCAAC |
| | CCGCTGCTGGGCCTGGACAGCACC |
| | (TandemPep.STAT3.DBD_codon optimized ORF) |
| 448 | SVDDHFAKSLGDTWLQIGGSGNPKTANVPQTVPMRLRKLPDSFFKPPE |
| | (Super TDU) |
| 449 | SVDDHFAKSLGDTWLQIGGSGNPKTANVPQTVPARLRKLPDSAFKPPE |
| | (Super TDU (MF2A)) |
| 450 | SVDDAAAKSLGDTWLQIGGSGNPKTANVPQTVPARLRKLPDSAFKPPE |
| | (Super TDU (HFMF4A)) |
| 451 | DPVVEEHFRRSLGKNYKEPEPAPNSVSITGSVDDHFAKALGDTWLQIKAAKD |
| | (Human VGLL4-TDU 1 and 2) |
| 452 | QTLPVASALSSHRTGPPPISPSKRKFSMEPGDEDLDCDNDHVSKMSRIFNPHL |
| | NKTANGDCRRDPRERSRSPIERAVAPTMSLHGSHLYTSLPSLGLEQPLALTKN |
| | SLDASRPAGLSPTLTPGERQQNRPSVITCASAGARNCNLSHCPIAHSGCAAPG |
| | PASYRRPPSAATTCDPVVEEHFRRSLGKNYKEPEPAPNSVSITGSVDDHFAKA |
| | LGDTWLQIKAAKDGASSSPESASRRGQPASPSAHMVSHSHSPSVVS |
| | (Human VGLL4) |
| 453 | MQTLPVASALSSHRTGPPPISPSKRKFSMEPGDEDLDCDNDHVSKMSRIFNPH |
| | LNKTANGDCRRDPRERSRSPIERAVAPTMSLHGSHLYTSLPSLGLEQPLALTK |
| | NSLDASRPAGLSPTLTPGERQQNRPSVITCASAGARNCNLSHCPIAHSGCAAP |
| | GPASYRRPPSAATTCDPVVEEHFRRSLGKNYKEPEPAPNSVSITGSVDDHFAK |
| | ALGDTWLQIKAAKDGASSSPESASRRGQPASPSAHMVSHSHSPSVVS |
| | (Human VGLL4 (complete)) |
| 454 | QTLPVASALSSHRTGPPPISPSKRKFSMEPGDEDLDCDNDHVSKMSRIFNPHL |
| | NKTANGDCRRDPRERSRSPIERAVAPTMSLHGSHLYTSLPSLGLEQPLALTKN |
| | SLDASRPAGLSPTLTPGERQQNRPSVITCASAGARNCNLSHCPIAHSGCAAPG |
| | PASYRRPPSAATTCDPVVEEAARRSLGKNYKEPEPAPNSVSITGSVDDAAAK |
| | ALGDTWLQIKAAKDGASSSPESASRRGQPASPSAHMVSHSHSPSVVS |
| | (Human VGLL4 (HF4A)) |
| 455 | MQTLPVASALSSHRTGPPPISPSKRKFSMEPGDEDLDCDNDHVSKMSRIFNPH |
| | LNKTANGDCRRDPRERSRSPIERAVAPTMSLHGSHLYTSLPSLGLEQPLALTK |
| | NSLDASRPAGLSPTLTPGERQQNRPSVITCASAGARNCNLSHCPIAHSGCAAP |
| | GPASYRRPPSAATTCDPVVEEAARRSLGKNYKEPEPAPNSVSITGSVDDAAA |
| | KALGDTWLQIKAAKDGASSSPESASRRGQPASPSAHMVSHSHSPSVVS |
| | (Human VGLL4 (HF4A) (complete)) |
| 456 | DPVVEEHFRRSLGKNYK (VGLL4 TDU 1 (V1)) |
| 457 | DPVVEEHFRRSLGKNYKE (VGLL4 TDU 1 (V2)) |
| 458 | DPVVEEHFRRSLGKNYKEPE (VGLL4 TDU 1 (V3)) |
| 459 | SVSITGSVDDHFAKALGDTWLQIK (VGLL4 TDU 2 (V1)) |
| 460 | SVSITGSVDDHFAKALGDTWLQIKA (VGLL4 TDU 2 (V2)) |
| 461 | SVSITGSVDDHFAKALGDTWLQIKAAKD (VGLL4 TDU 2 (V3)) |
| 462 | SVDDHFAKSLGDTWLQI (VGL44 Super TDU) |
| 463 | SVDDHFAKSLGDTWLQIGGSGNPKTANVPQTVPMRLRKLPDSFFKPPEM |
| | TYPRRRFRRRRHRPRSHLGQILRRRPWLVHPRHRYRWRRKNGIFNTGKPIPNP |
| | LLGLDST (Super TDU (NLS).cV5 ORF) |
| 464 | SVDDHFAKSLGDTWLQIGGSGNPKTANVPQTVPMRLRKLPDSFFKPPEP |
| | KKKRKVPKKKRKVPKKKRKVGIFNTGKPIPNPLLGLDST (Super TDU |
| | (sv40NLS).cV5 ORF) |
| 465 | SVDDHFAKSLGDTWLQIGGSGNPKTANVPQTVPARLRKLPDSAFKPPEM |
| | TYPRRRFRRRRHRPRSHLGQILRRRPWLVHPRHRYRWRRKNGIFNTGKPIPNP |
| | LLGLDST (MF2A(NLS).cV5 ORF) |
| 466 | VDDHFAKSLGDTWLQIGGSGNPKTANVPQTVPARLRKLPDSAFKPPEPK |
| | KKRKVPKKKRKVPKKKRKVGKPIPNPLLGLDST (MF2A(sv40NLS).cV5 ORF) |
| 467 | SVDDAAAKSLGDTWLQIGGSGNPKTANVPQTVPARLRKLPDSAFKPPEM |
| | TYPRRRFRRRRHRPRSHLGQILRRRPWLVHPRHRYRWRRKNGIFNTGKPIPNP |
| | LLGLDST (HFMF4A(NLS).cV5 ORF) |
| 468 | SVDDAAAKSLGDTWLQIGGSGNPKTANVPQTVPARLRKLPDSAFKPPEP |
| | KKKRKVPKKKRKVPKKKRKVGKPIPNPLLGLDST (HFMF4A(sv40NLS).cV5) |
| 469 | STMGDPVVEEHFRRSLGKNYKEPEPAPNSVSITGSVDDHFAKALGDTWLQ |
| | IKAAKDSAMTYPRRRFRRRRHRPRSHLGQILRRRPWLVHPRHRYRWRRKNG |
| | IFNTGKPIPNPLLGLDST (huVGLL4-TDU1.2(NLS).cV5 ORF) |
| 470 | STMGDPVVEEHFRRSLGKNYKEPEPAPNSVSITGSVDDHFAKALGDTWLQ |
| | IKAAKDSAPKKKRKVPKKKRKVPKKKRKVGKPIPNPLLGLDST (huVGLL4- |
| | TDU1.2(sv40NLS).cV5) |
| 471 | METPLDVLSRAASLVHADDEKREAALRGEPRIQTLPVASALSSHRTGPPPIS |
| | PSKRKFSMEPGDEDLDCDNDHVSKMSRIFNPHLNKTANGDCRRDPRERS |
| | RSPIERAVAPTMSLHGSHLYTSLPSLGLEQPLALTKNSLDASRPAGLSPTL |
| | TPGERQQNRPSVITCASAGARNCNLSHCPIAHSGCAAPGPASYRRPPSAA |
| | TTCDPVVEEHFRRSLGKNYKEPEPAPNSVSITGSVDDHFAKALGDTWLQI |
| | KAAKDGASSSPESASRRGQPASPSAHMVSHSHSPSVVSGKPIPNPLLGLDST |
| | (huVGLL4.cV5 ORF) |
| 472 | METPLDVLSRAASLVHADDEKREAALRGEPRIQTLPVASALSSHRTGPPPIS |
| | PSKRKFSMEPGDEDLDCDNDHVSKMSRIFNPHLNKTANGDCRRDPRERS |
| | RSPIERAVAPTMSLHGSHLYTSLPSLGLEQPLALTKNSLDASRPAGLSPTL |
| | TPGERQQNRPSVITCASAGARNCNLSHCPIAHSGCAAPGPASYRRPPSAA |
| | TTCDPVVEEAARRSLGKNYKEPEPAPNSVSITGSVDDAAAKALGDTWLQI |
| | KAAKDGASSSPESASRRGQPASPSAHMVSHSHSPSVVSGKPIPNPLLGLDST |
| | (huVGLL4(HF4A).cV5 ORF) |
| 473 | MIPRDPVVEEHFRRSLGKNYKEGLSEAKPATPEIQEIVDKVKPQLEEKTNET |
| | YGKLEAVQYKTQVLASTNYYIKVRAGDNKYMHLKVFNGPPGQNADRVLTG |
| | YQVDKNKDDELTGFPKKKRKVPKKKKVPKKKRKVGKPIPNPLLGLDST |
| | (SQT.(nt)VGLL4-TDU1.(sv40NLS).cV5 ORF) |
| 474 | MIPRGLSEAKPATPEIQEIVDKVKPQLEEKTNETYGKLEAVQYKTQVLDPVV |
| | EEHFRRSLGKNYKASTNYYIKVRAGDNKYMHLKVFNGPPGQNADRVLTGY |
| | QVDKNKDDELTGFPKKKRKVPKKKRVPKKKRKVGKPIPNPLLGLDST |
| | (SQT.(L1)VGLL4-TDU1.(sv40NLS).cV5 ORF) |
| 475 | MIPRGLSEAKPATPEIQEIVDKVKPQLEEKTNETYGKLEAVQYKTQVLASTNY |
| | YIKVRAGDNKYMHLKVFNGPDPVVEEHFRRSLGKNYKPGQNADRVLTGY |
| | QVDKNKDDELTGFPKKKRKVPKKKRVPKKKRKVGKPIPNPLLGLDST |
| | (SQT.(L2)VGLL4-TDU1.(sv40NLS).cV5 ORF) |
| 476 | MIPRSVSITGSVDDHFAKALGDTWLQIKGLSEAKPATPEIQEIVDKVKPQLE |
| | EKTNETYGKLEAVQYKTQVLASTNYYIKVRAGDNKYMHLKVFNGPPGQNA |
| | DRVLTGYQVDKNKDDELTGFPKKKRVPKKKRKVPKKKRKVGKPIPNPLLGL |
| | DST (SQT.(nt)VGLL4-TDU2.(sv40NLS).cV5 ORF) |
| 477 | MIPRGLSEAKPATPEIQEIVDKVKPQLEEKTNETYGKLEAVQYKTQVLSVSIT |
| | GSVDDHFAKALGDTWLQIKASTNYYIKVRAGDNKYMHLKVFNGPPGQNA |
| | DRVLTGYQVDKNKDDELTGFPKKKRVPKKKRKVPKKKRKVGKPIPNPLLGL |
| | DST (SQT.(L1)VGLL4-TDU2.(sv40NLS).cV5 ORF) |
| 478 | MIPRGLSEAKPATPEIQEIVDKVKPQLEEKTNETYGKLEAVQYKTQVLASTNY |
| | YIKVRAGDNKYMHLKVFNGPSVSITGSVDDHFAKALGDTWLQIKPGQNAD |
| | RVLTGYQVDKNKDDELTGFPKKKRVPKKKRKVPKKKRKVGKPIPNPLLGLD |
| | ST (SQT.(L2)VGLL4-TDU2.(sv40NLS).cV5 ORF) |
| 479 | STMGDPVVEEHFRRSLGKNYKEPEMTYPRRRFRRRRHRPRSHLGQILRRRP |
| | WLVHPRHRYRWRRKNGIFNTGKPIPNPLLGLDST (huVGLL4-TDU1(NLS).cV5 |
| | ORF) |
| 480 | STMSVSITGSVDDHFAKALGDTWLQIKAAKDSAMTYPRRRFRRRRHRPRS |
| | HLGQILRRRPWLVHPRHRYRWRRKNGIFNTGKPIPNPLLGLDST (huVGLL4- |
| | TDU2(NLS).cV5 ORF) |
| 481 | SVDDHFAKSLGDTWLQIGGSGNPKTANVPQTVPMRLRKLPDSFFKPPEM |
| | TYPRRRFRRRRHRPRSHLGQILRRRPWLVHPRHRYRWRRKNGIFNT |
| | (Super TDU (NLS) ORF amino acid sequence without v5 tag) |
| 482 | SVDDHFAKSLGDTWLQIGGSGNPKTANVPQTVPMRLRKLPDSFFKPPEP |
| | KKKRKVPKKKRKVPKKKRKVGIFNT (Super TDU (sv40NLS) ORF amino acid |
| | sequence without v5 tag) |
| 483 | SVDDHFAKSLGDTWLQIGGSGNPKTANVPQTVPARLRKLPDSAFKPPEM |
| | TYPRRRFRRRRHRPRSHLGQILRRRPWLVHPRHRYRWRRKNGIFNT |
| | (MF2A(NLS) ORF amino acid sequence without v5 tag) |
| 484 | SVDDHFAKSLGDTWLQIGGSGNPKTANVPQTVPARLRKLPDSAFKPPEPK |
| | KKRKVPKKKRKVPKKKRKV (MF2A(sv40NLS) ORF amino acid sequence |
| | without v5 tag) |
| 485 | SVDDAAAKSLGDTWLQIGGSGNPKTANVPQTVPARLRKLPDSAFKPPEM |
| | TYPRRRFRRRRHRPRSHLGQILRRRPWLVHPRHRYRWRRKNGIFNT |
| | (HFMF4A(NLS) ORF amino acid sequence without v5 tag) |
| 486 | SVDDAAAKSLGDTWLQIGGSGNPKTANVPQTVPARLRKLPDSAFKPPEP |
| | KKKRKVPKKKRKVPKKKRKV (HFMF4A(sv40NLS).ORF amino acid sequence |
| | without v5 tag) |
| 487 | STMGDPVVEEHFRRSLGKNYKEPEPAPNSVSITGSVDDHFAKALGDTWL |
| | QIKAAKDSAMTYPRRRFRRRRHRPRSHLGQILRRRPWLVHPRHRYRWRRKN |
| | GIFNT |
| | (huVGLL4-TDU1.2(NLS) ORF amino acid sequence without |
| | v5 tag) |
| 488 | STMGDPVVEEHFRRSLGKNYKEPEPAPNSVSITGSVDDHFAKALGDTWL |
| | QIKAAKDSAPKKKRKVPKKKRKVPKKKRKV |
| | (huVGLL4-TDU1.2(sv40NLS) ORF amino acid sequence without |
| | v5 tag) |
| 489 | METPLDVLSRAASLVHADDEKREAALRGEPRIQTLPVASALSSHRTGPPPIS |
| | PSKRKFSMEPGDEDLDCDNDHVSKMSRIFNPHLNKTANGDCRRDPRERS |
| | RSPIERAVAPTMSLHGSHLYTSLPSLGLEQPLALTKNSLDASRPAGLSPTL |
| | TPGERQQNRPSVITCASAGARNCNLSHCPIAHSGCAAPGPASYRRPPSAA |
| | TTCDPVVEEHFRRSLGKNYKEPEPAPNSVSITGSVDDHFAKALGDTWLQI |
| | KAAKDGASSSPESASRRGQPASPSAHMVSHSHSPSVVS |
| | (huVGLL4 ORF amino acid sequence without v5 tag) |
| 490 | METPLDVLSRAASLVHADDEKREAALRGEPRIQTLPVASALSSHRTGPPPIS |
| | PSKRKFSMEPGDEDLDCDNDHVSKMSRIFNPHLNKTANGDCRRDPRERS |
| | RSPIERAVAPTMSLHGSHLYTSLPSLGLEQPLALTKNSLDASRPAGLSPTL |
| | TPGERQQNRPSVITCASAGARNCNLSHCPIAHSGCAAPGPASYRRPPSAA |
| | TTCDPVVEEAARRSLGKNYKEPEPAPNSVSITGSVDDAAAKALGDTWLQI |
| | KAAKDGASSSPESASRRGQPASPSAHMVSHSHSPSVVS |
| | (huVGLL4(HF4A) ORF amino acid sequence without v5 tag) |
| 491 | MIPRDPVVEEHFRRSLGKNYKEGLSEAKPATPEIQEIVDKVKPQLEEKTNET |
| | YGKLEAVQYKTQVLASTNYYIKVRAGDNKYMHLKVFNGPPGQNADRVLTG |
| | YQVDKNKDDELTGFPKKKRKVPKKKKVPKKKRKV (SQT.(nt)VGLL4- |
| | TDU1.(sv4ONLS) ORF amino acid sequence without v5 tag) |
| 492 | MIPRGLS EAKPATPEIQEIVDKVKPQLEEKTNETYGKLEAVQYKTQVLDPVV |
| | EEHFRRSLGKNYKASTNYYIKVRAGDNKYMHLKVFNGPPGQNADRVLTGY |
| | QVDKNKDDELTGFPKKKRKVPKKKRVPKKKRKV (SQT.(L1)VGLL4- |
| | TDU1.(sv40NLS) ORF amino acid sequence without v5 tag) |
| 493 | MIPRGLSEAKPATPEIQEIVDKVKPQLEEKTNETYGKLEAVQYKTQVLASTNY |
| | YIKVRAGDNKYMHLKVFNGPDPVVEEHFRRSLGKNYKPGQNADRVLTGY |
| | QVDKNKDDELTGFPKKKRKVPKKKRVPKKKRKV (SQT.(L2)VGLL4- |
| | TDU1.(sv40NLS) ORF amino acid sequence without v5 tag) |
| 494 | MIPRSVSITGSVDDHFAKALGDTWLQIKGLSEAKPATPEIQEIVDKVKPQLE |
| | EKTNETYGKLEAVQYKTQVLASTNYYIKVRAGDNKYMHLKVFNGPPGQNA |
| | DRVLTGYQVDKNKDDELTGFPKKKRVPKKKRKVPKKKRKV |
| | (SQT.(nt)VGLL4-TDU2.(sv40NLS) ORF amino acid sequence |
| | without v5 tag) |
| 495 | MIPRGLSEAKPATPEIQEIVDKVKPQLEEKTNETYGKLEAVQYKTQVLSVSIT |
| | GSVDDHFAKALGDTWLQIKASTNYYIKVRAGDNKYMHLKVFNGPPGQNA |
| | DRVLTGYQVDKNKDDELTGFPKKKRVPKKKRKVPKKKRKV |
| | (SQT.(L1)VGLL4-TDU2.(sv40NLS) ORF amino acid sequence |
| | without v5 tag) |
| 496 | MIPRGLSEAKPATPEIQEIVDKVKPQLEEKTNETYGKLEAVQYKTQVLASTNY |
| | YIKVRAGDNKYMHLKVFNGPSVSITGSVDDHFAKALGDTWLQIKPGQNAD |
| | RVLTGYQVDKNKDDELTGFPKKKRVPKKKRKVPKKKRKV |
| | (SQT.(L2)VGLL4-TDU2.(sv40NLS) ORF amino acid sequence |
| | without v5 tag |
| 497 | STMGDPVVEEHFRRSLGKNYKEPEMTYPRRRFRRRRHRPRSHLGQILRRRP |
| | WLVHPRHRYRWRRKNGIFNT (huVGLL4-TDU1(NLS) ORF amino acid |
| | sequence without v5 tag) |
| 498 | STMSVSITGSVDDHFAKALGDTWLQIKAAKDSAMTYPRRRFRRRRHRPRS |
| | HLGQILRRRPWLVHPRHRYRWRRKNGIFNT (huVGLL4-TDU2(NLS) ORF |
| | amino acid sequence without v5 tag) |
| 499 | MSVDDHFAKSLGDTWLQIGSGVKQTLNFDLLKLAGDVESNPGPSVDDHFAK |
| | SLGDTWLQIGSGVKQTLNFDLLKLAGDVESNPGPSVDDHFAKSLGDTWLQI |
| | GKPIPNPLLGLDST (TandemPep.VGLL4.superTDU ORF) |
| 500 | MSVDDHFAKSLGDTWLQIGSGVKQTLNFDLLKLAGDVESNPGPDPVVEEHFR |
| | RSLGKNYKEGSGVKQTLNFDLLKLAGDVESNPGPDPVVEEHFRRSLGKNYKEG |
| | KPIPNPLLGLDST |
| | (TandemPep.VGLL4.TDU1 ORF) |
| 501 | MSVSITGSVDDHFAKALGDTWLQIKGSGVKQTLNFDLLKLAGDVESNPGPSV |
| | SITGSVDDHFAKALGDTWLQIKGSGVKQTLNFDLLKLAGDVESNPGPSVSIT |
| | GSVDDHFAKALGDTWLQIKGKPIPNPLLGLDST (TandemPep.VGLL4.TDU2 |
| | ORF) |
| 502 | MSVDDAAAKSLGDTWLQIGGSGNPKTANVPQTVPARLRKLPDSAFKPPE |
| | GSGVKQTLNFDLLKLAGDVESNPGPSVDDAAAKSLGDTWLQIGGSGNPKTA |
| | NVPQTVPARLRKLPDSAFKPPEGSGVKQTLNFDLLKLAGDVESNPGPSVDDA |
| | AAKSLGDTWLQIGGSGNPKTANVPQTVPARLRKLPDSAFKPPEGKPIPNP |
| | LLGLDST (TandemPep.HFMF4A ORF) |
| 503 | MSVDDHFAKSLGDTWLQIGGSGNPKTANVPQTVPARLRKLPDSAFKPPE |
| | GSGVKQTLNFDLLKLAGDVESNPGPSVDDHFAKSLGDTWLQIGGSGNPKTA |
| | NVPQTVPARLRKLPDSAFKPPEGSGVKQTLNFDLLKLAGDVESNPGPSVDDH |
| | FAKSLGDTWLQIGGSGNPKTANVPQTVPARLRKLPDSAFKPPEGKPIPNPL |
| | LGLDST (TandemPep.MF2A ORF) |
| 504 | MSVDDHFAKSLGDTWLQIGSGVKQTLNFDLLKLAGDVESNPGPSVDDHFAK |
| | SLGDTWLQIGSGVKQTLNFDLLKLAGDVESNPGPSVDDHFAKSLGDTWLQI |
| | (TandemPep.VGLL4.superTDU ORF amino acid sequence |
| | without v5 tag) |
| 505 | MDPVVEEHFRRSLGKNYKEGSGVKQTLNFDLLKLAGDVESNPGPDPVVEEH |
| | FRRSLGKNYKEGSGVKQTLNFDLLKLAGDVESNPGPDPVVEEHFRRSLGKN |
| | YKE |
| | (TandemPep.VGLL4.TDU2 ORF amino acid sequence without |
| | v5 tag) |
| 506 | MSVDDAAAKSLGDTWLQIGGSGNPKTANVPQTVPARLRKLPDSAFKPPE |
| | GSGVKQTLNFDLLKLAGDVESNPGPSVDDAAAKSLGDTWLQIGGSGNPKTA |
| | NVPQTVPARLRKLPDSAFKPPEGSGVKQTLNFDLLKLAGDVESNPGPSVDDA |
| | AAKSLGDTWLQIGGSGNPKTANVPQTVPARLRKLPDSAFKPPE |
| | (TandemPep.HFMF4A ORF amino acid sequence without |
| | v5 tag) |
| 507 | MSVDDHFAKSLGDTWLQIGGSGNPKTANVPQTVPARLRKLPDSAFKPPE |
| | GSGVKQTLNFDLLKLAGDVESNPGPSVDDHFAKSLGDTWLQIGGSGNPKTA |
| | NVPQTVPARLRKLPDSAFKPPEGSGVKQTLNFDLLKLAGDVESNPGPSVDDH |
| | FAKSLGDTWLQIGGSGNPKTANVPQTVPARLRKLPDSAFKPPE |
| | (TandemPep.MF2A ORF amino acid sequence without v5 |
| | tag) |
| 508 | ATGTCTGTGGATGATCATTTCGCCAAGAGCCTGGGAGATACATGGTTGCA |
| | GATCGGAGGGAGCGGCAATCCCAAAACAGCCAACGTTCCTCAAACCGTA |
| | CCAATGAGGCTCAGGAAACTGCCTGATAGCTTCTTCAAACCCCCTGAAAT |
| | GACCTACCCACGTAGGAGATTCAGAAGACGCAGACATCGGCCAAGGTCA |
| | CATCTTGGACAGATTCTGCGACGGCGCCCTTGGCTGGTGCATCCTCGGCAT |
| | CGGTATCGATGGCGGCGCAAAAACGGGATTTTTAACACCGGAAAACCAAT |
| | CCCCAATCCGCTGCTGGGGCTGGACAGTACG (Super TDU (NLS).cV5) |
| 509 | ATGAGTGTTGATGACCATTTTGCTAAATCACTGGGCGACACGTGGCTTCA |
| | GATAGGCGGAAGTGGGAATCCAAAAACCGCCAACGTGCCACAGACAGTC |
| | CCAATGCGCCTGCGCAAGCTTCCCGATAGCTTTTTTAAGCCCCCGGAACCC |
| | AAAAAAAAGAGAAAGGTGCCCAAAAAAAAACGGAAGGTACCTAAGAAG |
| | AAGCGCAAGGTCGGCATCTTCAACACAGGAAAACCAATACCTAATCCGCT |
| | TCTTGGCTTAGACAGTACC (Super TDU (sv40NLS).cV5) |
| 510 | ATGTCAGTGGACGATCACTTCGCTAAAAGTCTGGGAGACACATGGTTACA |
| | GATCGGCGGCTCTGGAAATCCAAAGACCGCCAACGTGCCTCAAACCGTCC |
| | CCGCTAGACTAAGAAAGCTCCCGGATTCAGCATTTAAACCACCTGAAATG |
| | ACTTATCCCAGACGTCGCTTCAGGCGGCGCCGGCACAGACCCCGCTCACA |
| | TCTGGGACAGATTCTCAGGCGGAGACCATGGCTTGTCCACCCCCGACACA |
| | GATACAGGTGGCGCAGAAAGAACGGTATCTTTAATACTGGCAAGCCAATA |
| | CCAAACCCGCTGCTCGGCCTGGATTCTACG (MF2A(NLS).cV5) |
| 511 | ATGTCAGTGGATGATCACTTTGCCAAGAGCCTAGGCGACACATGGCTGCA |
| | GATTGGTGGGAGTGGGAATCCCAAGACCGCCAATGTTCCCCAGACCGTGC |
| | CAGCGCGGCTTAGAAAACTTCCCGACTCCGCCTTTAAACCGCCTGAACCT |
| | AAAAAAAAAAGGAAAGTCCCGAAGAAGAAACGAAAAGTACCCAAGAAG |
| | AAGAGGAAAGTCGGCAAGCCCATTCCTAACCCTCTCTTGGGACTGGACTC |
| | CACC (MF2A(sv40NLS).cV5) |
| 512 | ATGTCCGTTGATGACGCCGCGGCAAAGAGCCTCGGAGATACCTGGCTCCA |
| | GATCGGTGGATCTGGAAACCCAAAGACCGCCAACGTGCCTCAGACTGTAC |
| | CTGCCCGCTTACGGAAATTGCCCGACTCCGCTTTCAAACCTCCTGAGATGA |
| | CCTATCCAAGGAGACGGTTCCGCAGACGACGCCACCGACCAAGGTCGCAT |
| | CTAGGGCAGATTCTGAGAAGGCGGCCATGGCTGGTCCACCCCAGGCATAG |
| | ATATAGGTGGCGGCGTAAAAATGGGATCTTTAACACAGGAAAACCGATCC |
| | CTAACCCACTGCTTGGACTGGACTCTACC (HFMF4A(NLS).cV5) |
| 513 | ATGTCCGTAGATGACGCCGCTGCGAAGTCACTAGGAGATACGTGGCTGCA |
| | AATTGGAGGGTCCGGCAACCCCAAGACAGCCAACGTACCACAGACGGTT |
| | CCAGCCCGCTTGCGTAAGCTGCCTGATTCTGCGTTCAAACCTCCGGAGCCT |
| | AAAAAGAAACGCAAAGTTCCTAAAAAAAAGCGCAAAGTGCCCAAGAAGA |
| | AGCGAAAAGTTGGAAAACCAATTCCCAACCCTCTGCTGGGTCTCGACTCC |
| | ACG (HFMF4A(sv40NLS).cV5) |
| 514 | ATGAGTACTATGGGAGATCCGGTGGTTGAAGAGCATTTCAGAAGAAGTCT |
| | CGGCAAAAACTACAAGGAACCAGAACCTGCTCCCAACAGCGTGTCTATCA |
| | CGGGTAGCGTCGATGATCATTTCGCAAAGGCACTCGGGGATACTTGGCTA |
| | CAGATTAAGGCGGCAAAGGATAGTGCTATGACTTACCCACGGCGCAGGTT |
| | CCGGCGCCGTAGGCACAGACCCAGGAGCCATTTGGGTCAGATACTCCGCC |
| | GCAGGCCATGGTTAGTGCACCCCAGACATAGATACAGGTGGAGGAGAAA |
| | GAACGGTATTTTCAACACTGGAAAGCCCATTCCCAATCCCCTGCTAGGCC |
| | TCGATTCCACT (huVGLL4-TDU1.2(NLS).cV5) |
| 515 | ATGAGTACTATGGGGGATCCCGTTGTAGAGGAGCATTTTCGCAGGAGTCT |
| | GGGTAAGAATTACAAAGAGCCTGAACCCGCGCCCAATAGCGTTTCCATCA |
| | CAGGAAGCGTTGACGATCACTTCGCGAAGGCTCTGGGTGACACGTGGCTC |
| | CAGATCAAAGCCGCAAAGGATTCCGCGCCCAAGAAGAAGCGGAAGGTGC |
| | CCAAGAAAAAAAGGAAGGTTCCGAAAAAAAAGAGGAAAGTTGGGAAGC |
| | CTATCCCCAACCCCCTACTGGGACTGGACAGCACC |
| | (huVGLL4-TDU1.2(sv40NLS).cV5) |
| 516 | ATGTCCACCATGGGAGACCCCGTCGTCGAAGAGCATTTTAGAAGGAGTCT |
| | CGGCAAAAACTATAAGGAACCAGAGATGACATATCCCCGGAGACGCTTC |
| | CGCCGGAGGCGGCATAGACCTCGGTCCCACCTCGGCCAGATCCTCCGCCG |
| | TAGGCCATGGCTTGTTCACCCACGGCATAGGTATAGATGGCGGAGGAAGA |
| | ATGGGATTTTTAACACA (huVGLL4-TDU1(NLS).cV5) |
| 517 | ATGTCTACAATGAGCGTGTCAATAACAGGTAGTGTAGATGACCACTTCGC |
| | TAAAGCTCTGGGGGATACGTGGCTTCAGATTAAAGCTGCTAAGGATAGTG |
| | CAATGACCTACCCCAGGCGCCGGTTCCGGCGACGTCGCCACCGCCCACGA |
| | TCTCATCTTGGGCAGATCTTACGCCGGCGCCCCTGGCTGGTTCATCCCCGA |
| | CATAGATACCGGTGGAGGAGGAAGAACGGCATTTTCAATACA (huVGLL4- |
| | TDU2(NLS).cV5) |
| 518 | ATGGAAACCCCACTCGACGTCCTATCTAGGGCAGCCAGCCTGGTGCACGC |
| | CGACGATGAAAAACGGGAAGCAGCCCTGCGGGGTGAACCGCGTATTCAA |
| | ACCTTGCCAGTAGCCTCAGCCTTAAGCAGTCATCGTACCGGTCCACCACC |
| | AATAAGCCCAAGTAAACGCAAATTCTCCATGGAGCCTGGAGATGAAGATC |
| | TTGACTGTGACAACGATCATGTGAGCAAAATGTCCCGCATCTTTAACCCTC |
| | ACCTGAATAAAACTGCTAATGGAGACTGTCGACGGGATCCTAGGGAAAG |
| | ATCTCGTTCACCTATTGAACGAGCTGTGGCCCCAACAATGAGTCTTCACG |
| | GCTCACACCTGTACACCTCTTTGCCATCTCTAGGACTCGAGCAGCCTCTCG |
| | CCTTGACTAAGAATTCACTGGATGCCTCAAGGCCCGCGGGGCTTAGTCCC |
| | ACACTGACCCCCGGTGAGCGGCAGCAGAATCGACCATCCGTTATCACCTG |
| | CGCCTCTGCAGGGGCAAGAAACTGTAACTTATCTCATTGCCCTATCGCAC |
| | ATAGTGGATGTGCCGCCCCCGGCCCAGCTTCTTATCGGCGGCCACCTAGC |
| | GCAGCAACCACTTGTGATCCAGTAGTGGAGGAGCATTTTAGGAGGTCCCT |
| | GGGTAAAAACTATAAAGAGCCAGAACCAGCGCCTAACAGTGTGTCCATTA |
| | CAGGCTCAGTGGATGATCATTTCGCAAAGGCTCTGGGGGATACATGGTTG |
| | CAGATCAAAGCCGCTAAAGACGGCGCCAGCTCCAGTCCGGAGTCTGCTTC |
| | GAGGCGGGGGCAGCCCGCGTCCCCTAGCGCCCATATGGTTAGCCATTCAC |
| | ATAGCCCCTCAGTTGTGTCTGGCAAACCTATCCCCAACCCTCTACTGGGTC |
| | TGGATAGCACT (huVGLL4.cV5) |
| 519 | ATGGAAACACCTCTGGACGTCCTGTCAAGAGCCGCCTCACTCGTTCACGC |
| | GGACGACGAGAAACGGGAGGCCGCACTGCGTGGCGAACCACGCATCCAG |
| | ACCCTACCCGTTGCTTCTGCGTTGTCCAGCCATAGAACCGGGCCCCCACCA |
| | ATCTCACCCTCTAAACGCAAGTTTTCCATGGAACCCGGTGACGAGGACTT |
| | GGACTGTGATAATGATCATGTGTCAAAAATGTCGCGCATCTTCAATCCAC |
| | ACCTGAACAAGACGGCTAATGGCGACTGTCGACGGGACCCTCGCGAGAG |
| | GTCCCGGAGCCCAATCGAACGTGCCGTGGCCCCTACTATGAGTCTCCACG |
| | GCTCACATCTATACACCTCTCTCCCGTCACTGGGTCTGGAACAACCACTCG |
| | CCCTCACCAAGAATAGCCTGGATGCATCCCGCCCTGCTGGCCTTAGTCCTA |
| | CTCTCACACCGGGTGAGAGGCAGCAGAACAGGCCCTCTGTCATAACCTGT |
| | GCATCAGCAGGCGCCCGCAATTGCAACCTGAGCCACTGTCCAATCGCCCA |
| | TAGTGGGTGCGCTGCCCCTGGTCCTGCTTCGTACAGGCGCCCCCCGTCCGC |
| | AGCCACAACGTGTGATCCCGTTGTGGAGGAAGCGGCCCGCAGATCCCTCG |
| | GCAAGAATTATAAGGAGCCCGAGCCCGCACCAAATAGTGTCAGCATCAC |
| | AGGGAGCGTCGACGACGCTGCTGCTAAGGCCCTGGGCGATACCTGGCTGC |
| | AGATCAAAGCGGCGAAAGATGGAGCATCCTCATCACCAGAGTCTGCCAGT |
| | AGAAGAGGCCAGCCTGCAAGTCCAAGTGCCCACATGGTATCACACTCCCA |
| | CAGTCCCTCCGTTGTGAGCGGAAAGCCTATCCCCAACCCACTGCTCGGGC |
| | TAGACTCTACC (huVGLL4(HF4A).cV5) |
| 520 | ATGATCCCACGAGATCCCGTGGTTGAGGAGCATTTTCGCAGGAGCCTGGG |
| | AAAAAATTACAAGGAAGGGCTCAGTGAAGCAAAGCCAGCCACCCCTGAA |
| | ATTCAGGAAATCGTTGACAAAGTTAAGCCTCAATTGGAGGAGAAGACTAA |
| | TGAGACTTACGGAAAATTGGAGGCAGTGCAGTACAAAACACAAGTCTTG |
| | GCTTCGACGAATTACTACATCAAGGTAAGAGCGGGGGATAATAAATATAT |
| | GCACCTGAAAGTTTTCAACGGCCCACCTGGTCAGAACGCGGACCGGGTAC |
| | TGACGGGCTATCAGGTGGATAAGAATAAGGACGATGAGCTGACAGGTTTC |
| | CCTAAAAAGAAGCGGAAAGTCCCAAAGAAGAAGCGGAAGGTGCCAAAG |
| | AAAAAACGAAAAGTTGGCAAGCCCATCCCTAACCCCCTCCTGGGCCTAGA |
| | (SQT.(nt)VGLL4-TDU1.(sv40NLS).cV5) |
| 521 | ATGATCCCACGCGGACTGTCCGAGGCCAAACCTGCCACCCCCGAAATCCA |
| | GGAAATCGTTGACAAGGTAAAACCTCAACTAGAAGAGAAAACCAATGAG |
| | ACTTACGGGAAACTGGAAGCCGTGCAGTACAAGACGCAGGTGCTCGATCC |
| | CGTGGTGGAAGAGCACTTTAGACGGAGTCTAGGCAAAAACTATAAAGCC |
| | AGTACGAACTATTACATAAAGGTGCGTGCCGGCGACAACAAATACATGCA |
| | CCTGAAAGTCTTCAACGGCCCTCCTGGGCAAAACGCCGATCGGGTTCTGA |
| | CAGGCTATCAGGTGGACAAAAATAAGGACGACGAGCTCACTGGCTTTCCT |
| | AAAAAGAAACGCAAGGTACCTAAAAAAAAGAGGAAAGTTCCCAAAAAG |
| | AAGCGGAAGGTGGGGAAGCCGATCCCAAACCCATTACTGGGGTTAGACT |
| | CTACA (SQT.(L1)VGLL4-TDU1.(sv40NLS).cV5) |
| 522 | ATGATCCCACGGGGGCTCTCTGAAGCCAAGCCAGCAACCCCCGAAATCCA |
| | AGAAATCGTCGACAAGGTGAAGCCACAGCTGGAAGAGAAGACTAACGAG |
| | ACATACGGGAAGCTCGAGGCAGTACAGTATAAAACACAGGTGCTGGCAA |
| | GTACTAATTACTATATTAAGGTCCGTGCCGGTGACAACAAGTATATGCAC |
| | CTGAAGGTCTTTAATGGCCCAGACCCTGTGGTCGAGGAGCACTTCAGACG |
| | GAGCTTGGGCAAGAACTATAAGCCGGGTCAGAACGCCGATCGAGTGCTG |
| | ACCGGGTACCAGGTTGATAAGAATAAAGATGATGAGCTTACTGGATTCCC |
| | AAAAAAAAAAAGAAAGGTGCCCAAAAAGAAGCGAAAAGTGCCTAAGAA |
| | GAAAAGAAAGGTAGGTAAGCCCATTCCAAACCCACTGCTAGGCCTCGATA |
| | GT (SQT.(L2)VGLL4-TDU1.(sv40NLS).cV5) |
| 523 | ATGATTCCCCGGTCAGTCTCGATTACCGGATCTGTGGACGACCACTTCGCT |
| | AAGGCATTGGGCGACACCTGGCTTCAGATTAAGGGTTTGAGCGAAGCCAA |
| | GCCAGCAACTCCTGAGATCCAAGAGATTGTGGATAAAGTCAAGCCACAGT |
| | TGGAAGAAAAGACAAATGAGACATACGGTAAGCTGGAGGCAGTCCAGTA |
| | CAAAACACAGGTCCTAGCATCTACAAATTATTACATAAAGGTGAGAGCTG |
| | GCGACAATAAGTACATGCACTTAAAGGTGTTCAACGGCCCGCCCGGTCAA |
| | AACGCCGACAGGGTGCTGACTGGGTATCAGGTCGACAAGAATAAAGATG |
| | ACGAGCTCACCGGCTTCCCTAAAAAGAAAAGGAAAGTGCCTAAGAAGAA |
| | ACGCAAAGTGCCAAAGAAAAAACGCAAGGTGGGCAAGCCTATCCCTAAT |
| | CCGCTCTTAGGCTTGGACTCCACT (SQT.(nt)VGLL4-TDU2.(sv40NLS).cV5) |
| 524 | ATGATTCCTAGGGGTCTTTCAGAAGCAAAGCCTGCCACACCCGAAATCCA |
| | GGAAATTGTAGACAAGGTCAAACCTCAACTGGAGGAGAAGACCAACGAA |
| | ACCTACGGTAAGCTGGAAGCCGTGCAGTACAAGACACAAGTTCTGTCAGT |
| | GTCCATTACCGGATCTGTGGATGACCATTTCGCTAAGGCACTGGGGGACA |
| | CTTGGCTGCAGATAAAGGCTTCTACAAATTATTATATTAAGGTGAGGGCT |
| | GGGGACAATAAGTACATGCACTTGAAGGTCTTTAATGGTCCCCCAGGGCA |
| | GAACGCTGACCGCGTGCTCACGGGATATCAGGTTGATAAAAACAAGGAC |
| | GACGAACTGACCGGATTCCCTAAAAAAAAACGGAAAGTTCCCAAGAAAA |
| | AACGAAAAGTGCCTAAAAAGAAGAGAAAGGTTGGCAAACCTATTCCCAA |
| | CCCTCTGCTTGGTCTCGACTCCACA (SQT.(L1)VGLL4-TDU2.(sv40NLS).cV5) |
| 525 | ATGATTCCCCGGGGTCTGAGTGAAGCTAAGCCAGCAACCCCTGAAATACA |
| | AGAAATCGTGGATAAGGTGAAGCCCCAGCTGGAGGAAAAAACAAACGAG |
| | ACGTACGGCAAGCTTGAGGCCGTGCAGTACAAAACCCAAGTGTTGGCTAG |
| | TACCAACTACTATATTAAGGTGAGGGCCGGCGATAATAAGTACATGCACC |
| | TGAAGGTGTTTAACGGACCCTCTGTGTCCATCACCGGATCCGTAGATGAC |
| | CACTTCGCTAAGGCGCTAGGCGATACCTGGCTGCAAATAAAACCCGGTCA |
| | GAACGCCGACAGAGTTTTAACTGGATATCAAGTCGACAAGAATAAGGAC |
| | GATGAGCTCACTGGCTTTCCCAAAAAAAAAAGAAAGGTTCCTAAGAAGA |
| | AAAGGAAAGTCCCCAAAAAGAAACGGAAGGTGGGTAAGCCAATCCCTAA |
| | TCCTCTGCTCGGACTAGATAGCACT |
| | (SQT.(L2)VGLL4-TDU2.(sv40NLS).cV5) |
| 526 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGAGC |
| | GTCGACGATCACTTCGCCAAGTCTCTGGGCGACACCTGGCTGCAGATCGG |
| | CAGCGGCGTGAAGCAAACCCTGAACTTCGACCTGCTGAAACTGGCCGGCG |
| | ATGTGGAGAGCAACCCCGGCCCGTCAGTGGACGACCACTTCGCCAAGAGC |
| | CTGGGAGACACCTGGCTGCAGATCGGCAGCGGCGTGAAGCAGACCTTAA |
| | ACTTCGACCTGCTGAAGCTGGCTGGTGACGTGGAGTCGAACCCCGGCCCC |
| | AGCGTGGACGACCACTTTGCCAAGTCACTGGGGGACACCTGGCTTCAAAT |
| | CGGCAAGCCCATCCCCAACCCCCTGCTGGGCCTGGACAGCACCTGATAAT |
| | AGGCTGGAGCCTCGGTGGCCATGCTTCTTGCCCCTTGGGCCTCCCCCCAGC |
| | CCCTCCTCCCCTTCCTGCACCCGTACCCCCGTGGTCTTTGAATAAAGTCTG |
| | AGTGGGCGGC (TandemPep.VGLL4.superTDU) |
| 527 | ATGAGCGTCGACGATCACTTCGCCAAGTCTCTGGGCGACACCTGGCTGCA |
| | GATCGGCAGCGGCGTGAAGCAAACCCTGAACTTCGACCTGCTGAAACTGG |
| | CCGGCGATGTGGAGAGCAACCCCGGCCCGTCAGTGGACGACCACTTCGCC |
| | AAGAGCCTGGGAGACACCTGGCTGCAGATCGGCAGCGGCGTGAAGCAGA |
| | CCTTAAACTTCGACCTGCTGAAGCTGGCTGGTGACGTGGAGTCGAACCCC |
| | GGCCCCAGCGTGGACGACCACTTTGCCAAGTCACTGGGGGACACCTGGCT |
| | TCAAATCGGCAAGCCCATCCCCAACCCCCTGCTGGGCCTGGACAGCACC |
| | (TandemPep.VGLL4.superTDU ORF) |
| 528 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGTCCG |
| | TCGACGATCACTTTGCGAAGTCACTCGGAGACACCTGGCTCCAAATCGGC |
| | TCCGGTGTCAAGCAGACCTTGAATTTCGACCTTCTGAAGCTGGCCGGCGA |
| | CGTGGAGAGCAACCCAGGCCCTTCGGTGGACGATCATTTCGCTAAGAGCC |
| | TGGGCGACACTTGGCTGCAAATCGGATCCGGAGTGAAGCAGACCCTGAAC |
| | TTCGATCTGCTCAAGCTCGCCGGAGATGTGGAATCCAACCCCGGACCTTC |
| | CGTGGACGACCACTTCGCCAAATCGCTGGGGGACACGTGGCTGCAGATCG |
| | GGAAGCCCATTCCGAACCCGCTGCTGGGTCTGGACAGCACCTGATAATAG |
| | GCTGGAGCCTCGGTGGCCATGCTTCTTGCCCCTTGGGCCTCCCCCCAGCCC |
| | CTCCTCCCCTTCCTGCACCCGTACCCCCGTGGTCTTTGAATAAAGTCTGAG |
| | TGGGCGGC (TandemPep.VGLL4.superTDU (codon optimized)) |
| 529 | ATGTCCGTCGACGATCACTTTGCGAAGTCACTCGGAGACACCTGGCTCCA |
| | AATCGGCTCCGGTGTCAAGCAGACCTTGAATTTCGACCTTCTGAAGCTGG |
| | CCGGCGACGTGGAGAGCAACCCAGGCCCTTCGGTGGACGATCATTTCGCT |
| | AAGAGCCTGGGCGACACTTGGCTGCAAATCGGATCCGGAGTGAAGCAGA |
| | CCCTGAACTTCGATCTGCTCAAGCTCGCCGGAGATGTGGAATCCAACCCC |
| | GGACCTTCCGTGGACGACCACTTCGCCAAATCGCTGGGGGACACGTGGCT |
| | GCAGATCGGGAAGCCCATTCCGAACCCGCTGCTGGGTCTGGACAGCACC |
| | (TandemPep.VGLL4.superTDU (codon optimized) ORF) |
| 530 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGGACC |
| | CCGTGGTGGAGGAGCACTTCAGGAGAAGCCTGGGCAAGAACTACAAGGA |
| | GGGCAGCGGCGTAAAGCAGACCCTGAACTTCGACCTACTGAAACTGGCCG |
| | GCGACGTGGAGAGCAACCCCGGTCCCGACCCTGTGGTGGAGGAGCACTTC |
| | CGAAGAAGCCTGGGCAAGAACTACAAGGAGGGCTCCGGCGTGAAGCAGA |
| | CCCTGAACTTCGATCTGCTGAAGCTGGCTGGCGATGTGGAAAGCAACCCC |
| | GGCCCCGATCCGGTGGTGGAGGAGCACTTCAGAAGATCCCTGGGCAAGA |
| | ACTACAAAGAGGGCAAGCCCATCCCCAACCCCCTGCTGGGCCTGGACAGC |
| | ACCTGATAATAGGCTGGAGCCTCGGTGGCCATGCTTCTTGCCCCTTGGGCC |
| | TCCCCCCAGCCCCTCCTCCCCTTCCTGCACCCGTACCCCCGTGGTCTTTGA |
| | ATAAAGTCTGAGTGGGCGGC (TandemPep.VGLL4.TDU1) |
| 531 | ATGGACCCCGTGGTGGAGGAGCACTTCAGGAGAAGCCTGGGCAAGAACT |
| | ACAAGGAGGGCAGCGGCGTAAAGCAGACCCTGAACTTCGACCTACTGAA |
| | ACTGGCCGGCGACGTGGAGAGCAACCCCGGTCCCGACCCTGTGGTGGAG |
| | GAGCACTTCCGAAGAAGCCTGGGCAAGAACTACAAGGAGGGCTCCGGCG |
| | TGAAGCAGACCCTGAACTTCGATCTGCTGAAGCTGGCTGGCGATGTGGAA |
| | AGCAACCCCGGCCCCGATCCGGTGGTGGAGGAGCACTTCAGAAGATCCCT |
| | GGGCAAGAACTACAAAGAGGGCAAGCCCATCCCCAACCCCCTGCTGGGC |
| | CTGGACAGCACC |
| | (TandemPep.VGLL4.TDU1 ORF) |
| 532 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGGATC |
| | CAGTGGTGGAGGAACATTTCCGCCGGTCACTGGGAAAGAACTATAAGGA |
| | GGGCTCCGGAGTGAAGCAGACTTTGAACTTTGATCTGCTGAAGCTGGCCG |
| | GCGACGTGGAAAGCAACCCTGGTCCTGATCCGGTCGTGGAGGAGCACTTC |
| | CGGAGAAGCCTGGGGAAGAACTACAAGGAAGGATCCGGCGTGAAGCAAA |
| | CCCTCAACTTCGACCTCCTGAAGCTCGCCGGGGACGTCGAGTCGAACCCC |
| | GGTCCCGACCCGGTGGTGGAAGAACACTTCAGGCGCTCCCTTGGAAAGAA |
| | TTACAAGGAGGGCAAACCGATCCCCAACCCTCTGCTGGGACTGGACAGCA |
| | CCTGATAATAGGCTGGAGCCTCGGTGGCCATGCTTCTTGCCCCTTGGGCCT |
| | CCCCCCAGCCCCTCCTCCCCTTCCTGCACCCGTACCCCCGTGGTCTTTGAA |
| | TAAAGTCTGAGTGGGCGGC (TandemPep.VGLL4.TDU1 (codon optimized)) |
| 533 | ATGGATCCAGTGGTGGAGGAACATTTCCGCCGGTCACTGGGAAAGAACTA |
| | TAAGGAGGGCTCCGGAGTGAAGCAGACTTTGAACTTTGATCTGCTGAAGC |
| | TGGCCGGCGACGTGGAAAGCAACCCTGGTCCTGATCCGGTCGTGGAGGAG |
| | CACTTCCGGAGAAGCCTGGGGAAGAACTACAAGGAAGGATCCGGCGTGA |
| | AGCAAACCCTCAACTTCGACCTCCTGAAGCTCGCCGGGGACGTCGAGTCG |
| | AACCCCGGTCCCGACCCGGTGGTGGAAGAACACTTCAGGCGCTCCCTTGG |
| | AAAGAATTACAAGGAGGGCAAACCGATCCCCAACCCTCTGCTGGGACTG |
| | GACAGCACC (TandemPep.VGLL4.TDU1 (codon optimized)ORF) |
| | TCAAGCTTTTGGACCCTCGTACAGAAGCTAATACGACTCACTATAGGGAA |
| | ATAAGAGAGAAAAGAAGAGTAAGAAGAAATATAAGAGCCACCATGAGC |
| | GTTAGTATTACCGGAAGCGTGGACGACCACTTCGCCAAGGCCCTGGGCGA |
| | CACCTGGCTGCAAATTAAGGGCAGCGGCGTAAAGCAGACCCTGAACTTCG |
| | ACCTTCTGAAGCTGGCTGGCGATGTGGAGAGTAACCCCGGCCCCAGCGTG |
| | AGCATAACCGGTTCAGTGGACGATCATTTCGCCAAGGCCCTTGGCGACAC |
| | GTGGCTGCAGATCAAAGGCTCCGGCGTGAAGCAGACCCTGAATTTCGATC |
| | TGCTGAAGTTGGCCGGCGACGTGGAGAGCAACCCCGGCCCCAGCGTGAG |
| | CATCACCGGCAGCGTGGACGACCACTTCGCCAAGGCCCTGGGCGACACCT |
| | GGCTGCAGATCAAGGGCAAGCCCATCCCCAACCCCCTGCTGGGCCTGGAC |
| | AGCACCTGATAATAGGCTGGAGCCTCGGTGGCCATGCTTCTTGCCCCTTG |
| | GGCCTCCCCCCAGCCCCTCCTCCCCTTCCTGCACCCGTACCCCCGTGGTCT |
| | TTGAATAAAGTCTGAGTGGGCGGC (TandemPep.VGLL4.TDU2) |
| 534 | ATGAGCGTTAGTATTACCGGAAGCGTGGACGACCACTTCGCCAAGGCCCT |
| | GGGCGACACCTGGCTGCAAATTAAGGGCAGCGGCGTAAAGCAGACCCTG |
| | AACTTCGACCTTCTGAAGCTGGCTGGCGATGTGGAGAGTAACCCCGGCCC |
| | CAGCGTGAGCATAACCGGTTCAGTGGACGATCATTTCGCCAAGGCCCTTG |
| | GCGACACGTGGCTGCAGATCAAAGGCTCCGGCGTGAAGCAGACCCTGAA |
| | TTTCGATCTGCTGAAGTTGGCCGGCGACGTGGAGAGCAACCCCGGCCCCA |
| | GCGTGAGCATCACCGGCAGCGTGGACGACCACTTCGCCAAGGCCCTGGGC |
| | GACACCTGGCTGCAGATCAAGGGCAAGCCCATCCCCAACCCCCTGCTGGG |
| | CCTGGACAGCACC |
| | (TandemPep.VGLL4.TDU2 ORF) |
| 535 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGTCGG |
| | TGTCAATCACCGGCTCCGTGGATGACCACTTCGCCAAGGCGCTTGGAGAT |
| | ACCTGGCTGCAGATTAAGGGGTCCGGCGTCAAGCAGACTCTCAACTTCGA |
| | TCTGCTCAAGCTGGCTGGCGATGTGGAGAGCAACCCGGGTCCCTCAGTGT |
| | CCATTACCGGATCCGTCGACGACCATTTCGCCAAAGCCCTGGGAGACACT |
| | TGGCTGCAAATCAAGGGGAGCGGAGTGAAGCAGACCCTGAACTTCGACC |
| | TGTTGAAGCTGGCCGGCGACGTGGAATCCAATCCGGGCCCTTCCGTGAGC |
| | ATCACCGGTTCGGTGGACGACCACTTTGCGAAGGCACTCGGAGACACGTG |
| | GCTCCAGATCAAGGGAAAGCCCATCCCTAACCCACTGCTGGGGCTGGACA |
| | GCACCTGATAATAGGCTGGAGCCTCGGTGGCCATGCTTCTTGCCCCTTGG |
| | GCCTCCCCCCAGCCCCTCCTCCCCTTCCTGCACCCGTACCCCCGTGGTCTT |
| | TGAATAAAGTCTGAGTGGGCGGC (TandemPep.VGLL4.TDU2_(codon |
| | optimized)) |
| 536 | ATGTCGGTGTCAATCACCGGCTCCGTGGATGACCACTTCGCCAAGGCGCT |
| | TGGAGATACCTGGCTGCAGATTAAGGGGTCCGGCGTCAAGCAGACTCTCA |
| | ACTTCGATCTGCTCAAGCTGGCTGGCGATGTGGAGAGCAACCCGGGTCCC |
| | TCAGTGTCCATTACCGGATCCGTCGACGACCATTTCGCCAAAGCCCTGGG |
| | AGACACTTGGCTGCAAATCAAGGGGAGCGGAGTGAAGCAGACCCTGAAC |
| | TTCGACCTGTTGAAGCTGGCCGGCGACGTGGAATCCAATCCGGGCCCTTC |
| | CGTGAGCATCACCGGTTCGGTGGACGACCACTTTGCGAAGGCACTCGGAG |
| | ACACGTGGCTCCAGATCAAGGGAAAGCCCATCCCTAACCCACTGCTGGGG |
| | CTGGACAGCACC |
| | (TandemPep.VGLL4.TDU2 (codon optimized)ORF) |
| 537 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGTCCG |
| | TGGACGACGCCGCCGCGAAGAGCCTGGGCGACACCTGGCTGCAGATCGG |
| | CGGAAGCGGCAATCCCAAGACCGCCAACGTGCCCCAAACCGTGCCCGCCC |
| | GCCTGAGAAAACTGCCCGATAGCGCCTTCAAGCCGCCGGAGGGTAGCGG |
| | CGTGAAGCAAACCCTGAACTTCGACCTTCTGAAGCTGGCTGGCGACGTAG |
| | AAAGCAACCCCGGCCCTAGCGTTGACGACGCCGCCGCCAAGAGCCTGGGT |
| | GACACATGGCTACAGATAGGCGGCTCGGGCAACCCCAAGACTGCCAATGT |
| | GCCCCAGACCGTGCCTGCTAGACTGAGAAAACTGCCCGACAGCGCCTTCA |
| | AGCCTCCTGAAGGGAGCGGGGTGAAGCAGACCTTGAACTTTGATCTGTTG |
| | AAGCTGGCCGGCGATGTAGAGAGCAATCCTGGCCCCAGCGTGGACGACG |
| | CTGCCGCCAAGTCACTGGGCGACACCTGGTTACAGATTGGTGGCAGCGGT |
| | AACCCCAAAACCGCCAACGTCCCACAGACCGTACCCGCCAGACTGAGAA |
| | AGCTGCCCGACAGCGCCTTCAAGCCCCCCGAGGGCAAGCCCATTCCCAAC |
| | CCCCTGCTGGGGCTGGACAGCACCTGATAATAGGCTGGAGCCTCGGTGGC |
| | CATGCTTCTTGCCCCTTGGGCCTCCCCCCAGCCCCTCCTCCCCTTCCTGCAC |
| | CCGTACCCCCGTGGTCTTTGAATAAAGTCTGAGTGGGCGGC |
| | (TandemPep.HFMF4A) |
| 538 | ATGTCCGTGGACGACGCCGCCGCGAAGAGCCTGGGCGACACCTGGCTGCA |
| | GATCGGCGGAAGCGGCAATCCCAAGACCGCCAACGTGCCCCAAACCGTG |
| | CCCGCCCGCCTGAGAAAACTGCCCGATAGCGCCTTCAAGCCGCCGGAGGG |
| | TAGCGGCGTGAAGCAAACCCTGAACTTCGACCTTCTGAAGCTGGCTGGCG |
| | ACGTAGAAAGCAACCCCGGCCCTAGCGTTGACGACGCCGCCGCCAAGAG |
| | CCTGGGTGACACATGGCTACAGATAGGCGGCTCGGGCAACCCCAAGACTG |
| | CCAATGTGCCCCAGACCGTGCCTGCTAGACTGAGAAAACTGCCCGACAGC |
| | GCCTTCAAGCCTCCTGAAGGGAGCGGGGTGAAGCAGACCTTGAACTTTGA |
| | TCTGTTGAAGCTGGCCGGCGATGTAGAGAGCAATCCTGGCCCCAGCGTGG |
| | ACGACGCTGCCGCCAAGTCACTGGGCGACACCTGGTTACAGATTGGTGGC |
| | AGCGGTAACCCCAAAACCGCCAACGTCCCACAGACCGTACCCGCCAGACT |
| | GAGAAAGCTGCCCGACAGCGCCTTCAAGCCCCCCGAGGGCAAGCCCATTC |
| | CCAACCCCCTGCTGGGGCTGGACAGCACC (TandemPep.HFMF4A ORF) |
| 539 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGAGC |
| | GTGGATGATGCAGCGGCGAAGTCCCTGGGGGATACATGGCTTCAGATTGG |
| | TGGATCGGGGAACCCTAAGACTGCCAACGTCCCGCAAACTGTGCCTGCTC |
| | GCCTGAGGAAGCTCCCGGACTCTGCCTTCAAACCCCCTGAAGGCTCAGGA |
| | GTCAAGCAGACTCTCAACTTTGACCTCCTTAAGCTGGCCGGGGATGTGGA |
| | GTCGAACCCCGGGCCTTCCGTCGACGACGCCGCTGCTAAGTCCCTCGGAG |
| | ACACCTGGCTGCAGATCGGCGGATCGGGCAACCCCAAGACGGCCAATGT |
| | GCCACAGACCGTGCCCGCCCGGCTGAGAAAGCTGCCGGACAGCGCGTTCA |
| | AGCCTCCTGAAGGATCCGGCGTGAAGCAGACCCTGAACTTCGACTTGCTG |
| | AAACTGGCCGGCGACGTGGAATCAAACCCGGGACCGAGCGTGGACGACG |
| | CGGCCGCCAAATCCCTGGGCGACACTTGGCTCCAAATCGGTGGATCCGGC |
| | AATCCCAAGACCGCCAACGTGCCCCAGACCGTCCCGGCCCGGCTGCGCAA |
| | GTTGCCAGATTCCGCATTCAAGCCGCCTGAGGGAAAGCCGATCCCGAACC |
| | CCCTGCTCGGTCTGGATAGCACCTGATAATAGGCTGGAGCCTCGGTGGCC |
| | ATGCTTCTTGCCCCTTGGGCCTCCCCCCAGCCCCTCCTCCCCTTCCTGCACC |
| | CGTACCCCCGTGGTCTTTGAATAAAGTCTGAGTGGGCGGC |
| | (TandemPep.HFMF4A (codon optimized)) |
| 540 | ATGAGCGTGGATGATGCAGCGGCGAAGTCCCTGGGGGATACATGGCTTCA |
| | GATTGGTGGATCGGGGAACCCTAAGACTGCCAACGTCCCGCAAACTGTGC |
| | CTGCTCGCCTGAGGAAGCTCCCGGACTCTGCCTTCAAACCCCCTGAAGGC |
| | TCAGGAGTCAAGCAGACTCTCAACTTTGACCTCCTTAAGCTGGCCGGGGA |
| | TGTGGAGTCGAACCCCGGGCCTTCCGTCGACGACGCCGCTGCTAAGTCCC |
| | TCGGAGACACCTGGCTGCAGATCGGCGGATCGGGCAACCCCAAGACGGC |
| | CAATGTGCCACAGACCGTGCCCGCCCGGCTGAGAAAGCTGCCGGACAGC |
| | GCGTTCAAGCCTCCTGAAGGATCCGGCGTGAAGCAGACCCTGAACTTCGA |
| | CTTGCTGAAACTGGCCGGCGACGTGGAATCAAACCCGGGACCGAGCGTG |
| | GACGACGCGGCCGCCAAATCCCTGGGCGACACTTGGCTCCAAATCGGTGG |
| | ATCCGGCAATCCCAAGACCGCCAACGTGCCCCAGACCGTCCCGGCCCGGC |
| | TGCGCAAGTTGCCAGATTCCGCATTCAAGCCGCCTGAGGGAAAGCCGATC |
| | CCGAACCCCCTGCTCGGTCTGGATAGCACC (TandemPep.HFMF4A |
| | (codon optimized)ORF) |
| 541 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGAGC |
| | GTGGACGACCACTTCGCCAAGAGCCTGGGCGACACCTGGTTACAGATAGG |
| | CGGGTCTGGCAATCCTAAGACCGCGAACGTTCCCCAAACCGTCCCGGCCA |
| | GACTGAGAAAACTGCCAGATAGCGCCTTCAAGCCCCCCGAAGGTAGCGG |
| | CGTCAAACAGACCCTTAATTTCGATTTACTGAAGCTCGCCGGCGACGTGG |
| | AGTCGAACCCCGGCCCCAGCGTGGACGATCACTTCGCTAAGTCGCTGGGC |
| | GACACCTGGCTGCAGATTGGCGGCTCGGGAAACCCCAAGACCGCCAACGT |
| | GCCCCAGACTGTGCCCGCTAGACTCAGAAAGCTTCCTGACAGCGCCTTCA |
| | AGCCCCCCGAAGGCAGCGGAGTTAAACAAACCTTAAACTTCGACCTACTG |
| | AAACTGGCCGGGGACGTGGAGAGCAATCCCGGTCCCTCCGTCGACGATCA |
| | CTTCGCGAAGAGCCTGGGCGATACCTGGCTTCAGATCGGCGGCAGCGGCA |
| | ATCCAAAGACAGCCAACGTGCCACAGACCGTGCCCGCCAGACTGAGAAA |
| | GCTGCCCGACTCGGCCTTCAAGCCACCTGAGGGCAAACCCATCCCCAACC |
| | CCCTGCTGGGACTGGACAGCACCTGATAATAGGCTGGAGCCTCGGTGGCC |
| | ATGCTTCTTGCCCCTTGGGCCTCCCCCCAGCCCCTCCTCCCCTTCCTGCACC |
| | CGTACCCCCGTGGTCTTTGAATAAAGTCTGAGTGGGCGGC |
| | (TandemPep.MF2A) |
| 542 | ATGAGCGTGGACGACCACTTCGCCAAGAGCCTGGGCGACACCTGGTTACA |
| | GATAGGCGGGTCTGGCAATCCTAAGACCGCGAACGTTCCCCAAACCGTCC |
| | CGGCCAGACTGAGAAAACTGCCAGATAGCGCCTTCAAGCCCCCCGAAGGT |
| | AGCGGCGTCAAACAGACCCTTAATTTCGATTTACTGAAGCTCGCCGGCGA |
| | CGTGGAGTCGAACCCCGGCCCCAGCGTGGACGATCACTTCGCTAAGTCGC |
| | TGGGCGACACCTGGCTGCAGATTGGCGGCTCGGGAAACCCCAAGACCGCC |
| | AACGTGCCCCAGACTGTGCCCGCTAGACTCAGAAAGCTTCCTGACAGCGC |
| | CTTCAAGCCCCCCGAAGGCAGCGGAGTTAAACAAACCTTAAACTTCGACC |
| | TACTGAAACTGGCCGGGGACGTGGAGAGCAATCCCGGTCCCTCCGTCGAC |
| | GATCACTTCGCGAAGAGCCTGGGCGATACCTGGCTTCAGATCGGCGGCAG |
| | CGGCAATCCAAAGACAGCCAACGTGCCACAGACCGTGCCCGCCAGACTG |
| | AGAAAGCTGCCCGACTCGGCCTTCAAGCCACCTGAGGGCAAACCCATCCC |
| | CAACCCCCTGCTGGGACTGGACAGCACC (TandemPep.MF2A ORF) |
| 543 | TCAAGCTTTTGGACCCTCGTACAGAAGCTAATACGACTCACTATAGGGAA |
| | ATAAGAGAGAAAAGAAGAGTAAGAAGAAATATAAGAGCCACCATGTCCG |
| | TGGATGACCACTTTGCTAAGTCGCTGGGAGATACCTGGCTGCAGATCGGA |
| | GGCTCCGGGAACCCAAAGACTGCGAACGTGCCCCAGACCGTGCCGGCCC |
| | GGCTGAGGAAGCTGCCTGACTCCGCGTTCAAGCCGCCCGAAGGATCCGGA |
| | GTGAAGCAGACGCTGAATTTCGATCTGCTCAAGCTGGCCGGCGACGTGGA |
| | GTCGAACCCGGGTCCGAGCGTGGACGATCATTTCGCAAAGTCACTCGGAG |
| | ACACTTGGCTGCAAATTGGGGGCAGCGGCAACCCTAAAACCGCCAACGTG |
| | CCACAGACTGTGCCGGCTAGATTGCGGAAGCTCCCTGACTCGGCCTTCAA |
| | GCCCCCTGAAGGCAGCGGGGTCAAGCAGACCCTCAACTTCGACCTTCTCA |
| | AGTTGGCGGGAGATGTCGAGTCAAACCCAGGACCGTCCGTCGACGATCAC |
| | TTCGCCAAATCCCTGGGCGACACCTGGCTTCAAATCGGCGGTTCCGGAAA |
| | CCCTAAGACTGCAAACGTCCCTCAAACCGTGCCCGCCCGCCTGCGCAAGC |
| | TGCCGGACAGCGCCTTCAAACCGCCCGAGGGAAAGCCCATCCCCAATCCC |
| | CTGCTGGGTCTGGACAGCACCTGATAATAGGCTGGAGCCTCGGTGGCCAT |
| | GCTTCTTGCCCCTTGGGCCTCCCCCCAGCCCCTCCTCCCCTTCCTGCACCC |
| | GTACCCCCGTGGTCTTTGAATAAAGTCTGAGTGGGCGGC |
| | (TandemPep.MF2A (co-op'd)) |
| 544 | ATGTCCGTGGATGACCACTTTGCTAAGTCGCTGGGAGATACCTGGCTGCA |
| | GATCGGAGGCTCCGGGAACCCAAAGACTGCGAACGTGCCCCAGACCGTG |
| | CCGGCCCGGCTGAGGAAGCTGCCTGACTCCGCGTTCAAGCCGCCCGAAGG |
| | ATCCGGAGTGAAGCAGACGCTGAATTTCGATCTGCTCAAGCTGGCCGGCG |
| | ACGTGGAGTCGAACCCGGGTCCGAGCGTGGACGATCATTTCGCAAAGTCA |
| | CTCGGAGACACTTGGCTGCAAATTGGGGGCAGCGGCAACCCTAAAACCGC |
| | CAACGTGCCACAGACTGTGCCGGCTAGATTGCGGAAGCTCCCTGACTCGG |
| | CCTTCAAGCCCCCTGAAGGCAGCGGGGTCAAGCAGACCCTCAACTTCGAC |
| | CTTCTCAAGTTGGCGGGAGATGTCGAGTCAAACCCAGGACCGTCCGTCGA |
| | CGATCACTTCGCCAAATCCCTGGGCGACACCTGGCTTCAAATCGGCGGTT |
| | CCGGAAACCCTAAGACTGCAAACGTCCCTCAAACCGTGCCCGCCCGCCTG |
| | CGCAAGCTGCCGGACAGCGCCTTCAAACCGCCCGAGGGAAAGCCCATCCC |
| | CAATCCCCTGCTGGGTCTGGACAGCACC |
| | (TandemPep.MF2A (co-op'd) ORF) |
| 545 | GKPIPNPLLGLDST (V5 epitope tag) |
| 546 | MGTMENLSRRLKVTGDLFDIMSGSGVKQTLNFDLLKLAGDVESNPGPGTM |
| | ENLSRRLKVTGDLFDIMSGSGVKQTLNFDLLKLAGDVESNPGPGTMENLS |
| | RRLKVTGDLFDIMS (BeclinBH3x3.F2A, no epitope tag) |
| 547 | MGTMENLSRRLKVTGDLFDIMSGSGVKQTLNFDLLKLAGDVESNPGPGTM |
| | ENLSRRLKVTGDLFDIMSGSGVKQTLNFDLLKLAGDVESNPGPGTMENLS |
| | RRLKVTGDLFDIMSGKPIPNPLLGLDST |
| | (BeclinBH3x3.F2A, V5 epitope tag) |
| 548 | ATGGGCACCATGGAGAATCTTAGCAGACGACTGAAAGTGACCGGCGATCT |
| | GTTCGACATCATGAGCGGCAGCGGCGTGAAGCAGACCCTGAACTTTGACT |
| | TGCTGAAGCTGGCCGGCGACGTGGAAAGCAACCCCGGACCCGGCACCAT |
| | GGAGAACCTGAGCCGGCGGCTGAAGGTGACCGGCGACTTGTTCGACATCA |
| | TGAGCGGCAGCGGAGTGAAGCAGACTTTGAACTTCGACCTTCTGAAACTG |
| | GCCGGCGATGTGGAGAGCAATCCAGGCCCGGGCACCATGGAGAATCTGA |
| | GCAGAAGACTGAAGGTGACTGGCGACCTGTTCGACATTATGAGCGGCAA |
| | GCCCATCCCCAACCCCCTGCTGGGTCTGGATAGCACC |
| | (BeclinBH3x3.F2A, V5 epitope tag) |
| 549 | ATGGGAACTATGGAGAACCTGTCGCGGAGGTTGAAAGTGACCGGCGACC |
| | TGTTTGACATTATGTCCGGCTCCGGAGTGAAGCAGACCCTGAACTTCGAC |
| | CTTTTGAAGCTGGCCGGCGACGTGGAATCGAACCCAGGCCCTGGTACTAT |
| | GGAAAACCTCAGCAGACGCCTGAAAGTCACCGGAGATCTGTTCGACATCA |
| | TGAGCGGATCCGGCGTGAAGCAAACTCTGAATTTCGACCTCCTGAAGCTT |
| | GCGGGAGATGTGGAGTCAAACCCGGGGCCCGGTACCATGGAAAATCTGT |
| | CCCGCCGGCTCAAGGTCACCGGGGACCTGTTCGATATCATGTCCGGGAAG |
| | CCTATCCCCAACCCGCTGCTGGGACTCGACAGCACC |
| | (BeclinBH3x3.F2A, V5 epitope tag) |

1. A messenger RNA (mRNA) encoding at least one intracellular binding domain, wherein said mRNA comprises one or more modified nucleobases.

2. (canceled)

3. The mRNA of claim 1, wherein the at least one intracellular binding domain is selected from the group consisting of: a Bcl-2 homology (BH3) domain, a TOPK inhibitory peptide, a SALL4 inhibitory peptide, a Ras inhibitory peptide, a p53 inhibitory peptide, a PP2a inhibitory peptide, a STAT3 inhibitory peptide and a YAP inhibitory peptide.

4. (canceled)

5. The mRNA of claim 1, which encodes two to ten intracellular binding domains.

6. (canceled)

7. The mRNA of claim 5, wherein the BH3 domains are selected from the group consisting of PUMA BH3, Bim BH3, Bad BH3, Noxa BH3, Beclin BH3, truncated BID containing a BH3 domain, and combinations thereof.

8-9. (canceled)

10. The mRNA of claim 1, which further encodes a linker located between each intracellular binding domain.

11. (canceled)

12. The mRNA of claim 10, wherein the linker is a cleavable linker selected from the group consisting of F2A linker, P2A linker, T2A linker, E2A linker, a gly-ser linker and combinations thereof.

13-14. (canceled)

15. The mRNA of claim 1, which further comprises one or more microRNA (miR) binding sites.

16-17. (canceled)

18. The mRNA of claim 15, which comprises an miR122 binding site, an miR142.3p binding site, or a combination of both.

19. The mRNA of claim 18, which further comprises at least one IRES sequence.

20-50. (canceled)

51. The mRNA of claim 1, wherein the at least one chemically modified nucleobase is selected from the group consisting of pseudouracil (ψ), N1-methylpseudouracil (m1ψ), 2-thiouracil (s2U), 4′-thiouracil, 5-methylcytosine, 5-methyluracil, and any combination thereof.

52-53. (canceled)

54. The mRNA of claim 51, wherein the mRNA further comprises a 5′ UTR.

55. The mRNA of claim 54, wherein the 5′ UTR comprises a nucleic acid sequence at least 90%, at least about 95%, at least about 96%, at least about 97%, at least about 98%, at least about 99%, or about 100% identical to a 5′UTR sequence selected from the group consisting of SEQ ID NO: 327-351, or any combination thereof.

56. The mRNA of claim 54, wherein the mRNA further comprises a 3′ UTR, wherein the 3′ UTR comprises a nucleic acid sequence at least about 90%, at least about 95%, at least about 96%, at least about 97%, at least about 98%, at least about 99%, or about 100% identical to a 3′UTR sequence selected from the group consisting of SEQ ID NO: 352-369, or any combination thereof, and wherein the mRNA comprises a microRNA binding site located within the 3′ UTR.

57-58. (canceled)

59. The mRNA of claim 56, wherein the mRNA further comprises a 5′ terminal cap, wherein the 5′ terminal cap comprises a Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

60. (canceled)

61. The mRNA of claim 59, wherein the mRNA further comprises a poly-A region, has about 10 to about 200, about 20 to about 180, about 50 to about 160, about 70 to about 140, about 80 to about 120 nucleotides in length.

62-63. (canceled)

64. The mRNA of claim 61, wherein upon administration to a subject, the mRNA has:
(i) a longer plasma half-life;
(ii) increased expression of at least one intracellular binding domain encoded by the ORF;
(iii) a lower frequency of arrested translation resulting in an expression fragment;
(iv) greater structural stability; or
(v) any combination thereof,
relative to a corresponding mRNA encoding the at least one intracellular binding domain.

65-102. (canceled)

103. A lipid nanoparticle comprising the mRNA of claim 1.

104. (canceled)

105. The lipid nanoparticle of claim 103, wherein the lipid nanoparticle comprises a cationic and/or ionizable lipid.

106. The lipid nanoparticle of claim 105, wherein the cationic and/or ionizable lipid is DLin-KC2-DMA or DLin-MC3-DMA.

107-108. (canceled)

109. A pharmaceutical composition comprising the lipid nanoparticle of claim 103, and a pharmaceutically acceptable carrier, diluent or excipient.

110-118. (canceled)

119. A method for treating a cancer in a subject in need thereof, the method comprising inducing apoptosis in a cell by contacting the cell with an effective amount of the mRNA of claim 1.

120. (canceled)

121. The method of claim 119, wherein the subject is a human.

122. The method of claim 121, wherein the cancer is liver cancer, colorectal cancer, hepatocellular carcinoma, acute myeloid leukemia, chronic myeloid leukemia, chronic myleomonocytic leukemia, myelodysplastic syndrome, myeloproliferative disease, a primary tumor, or a metastasis.

123-126. (canceled)

127. The mRNA of claim 1, further encoding a scaffold polypeptide selected from a group comprising wild-type SteA, STM, SQM, and SQT.
